(19) United States
(12) Patent Application Publication
(10) Pub. No.: US 2024/0246918 A1
(43) Pub. Date: Jul. 25, 2024

(54) COMPOUNDS AND COMPOSITIONS AS EIF4E INHIBITORS AND THE USES THEREOF

(71) Applicant: Ribometrix, Inc., Durham, NC (US)

(72) Inventors: Rajeev BHIDE, Princeton, NJ (US); Roheeth Kumar PAVANA, Morrisville, NC (US); Stephen THOMSON, Durham, NC (US); Ashis Kumar SAHA, Framingham, MA (US); Ramamurty V S CHANGALVALA, Morrisville, NC (US); Stevan DJURIC, Durham, NC (US); Ramesh Kumar SISTLA, Kkarnataka (IN); Sundara Raghuram TANGIRALA, Telangana (IN); Laxmikant Shamlal DATRANGE, Telangana (IN); Samir Satish KHER, Telangana (IN); Saumya ROY, Telangana (IN); Suparna GUPTA, Plymouth, MN (US); Michael PATANE, Durham, NC (US); Ratnakumar SREEKANTHA, Telangana (IN); Simon HAYDER, Zionsville, IN (US)

(21) Appl. No.: 18/508,102

(22) Filed: Nov. 13, 2023

Related U.S. Application Data
(60) Provisional application No. 63/424,463, filed on Nov. 10, 2022.

(51) Int. Cl. C07D 239/90; C07D 495/04; C07D 519/00

(52) U.S. Cl. CPC C07D 239/90; C07D 495/04; C07D 519/00

(57) ABSTRACT

Described herein are compounds of Formula I, and their pharmaceutically acceptable salts, solvates, stereoisomers, or prodrugs, as well as their uses (e.g., as eIF4E inhibitors).

### RELATED APPLICATIONS

This application claims the benefit of and priority to U.S. Provisional Application No. 63/424,463, filed Nov. 10, 2022, the contents of which are incorporated herein by reference in their entirety.

### BACKGROUND

Eukaryotic initiation factor 4E (eIF4E) is the limiting protein factor for initiation of mRNA translation. For example, eIF4E is shown to modulate the translation of cyclin D mRNA. eIF4E initiates translation by binding to the 7-methylguanosine cap at the 5′ end of mRNAs, recruiting the other members of the eIF4F complex, including the scaffolding protein, eIF4G, and the RNA helicase, eIF4A. Once assembled at the 5′ end of mRNAs, the eIF4F complex then recruits additional translation initiation factor factors, culminating in ribosome recruitment and initiation of protein translation.

Under basal conditions, the activity of eIF4E is regulated by multiple mechanisms, including binding to and sequestration by the abundant negative regulatory proteins, the 4E binding proteins (4EBPs). In cancer, eIF4E activity is elevated through several mechanisms including mutational activation of oncogenic signaling pathways such as receptor tyrosine kinases (RTK), RAS/RAF family members, PI3K family members, and others that converge on eIF4E (REF). Dysregulated expression of eIF4E itself is pro-tumorigenic, underscoring its important role in cell transformation and cancer formation. Additionally, elevated expression in patients has been shown to lead to poor prognosis in multiple indications including breast, head and neck, ovarian and colorectal cancers. Up-regulation of eIF4E activity is also reported to be important for evolution of resistance to chemotherapeutic and targeted cancer agents. Lastly, genetic inhibition of eIF4E as well as disruption of other facets of the MNK1-eIF4E axis have demonstrated antitumor efficacy in multiple preclinical models including melanoma, ovarian, esophageal, lung, and breast cancer.

Therefore, pharmacological inhibition of eIF4E activity has the potential to be an effective anti-neoplastic therapy.

### SUMMARY

In certain aspects, the present disclosure provides compounds of Formula I, I′, or I″:

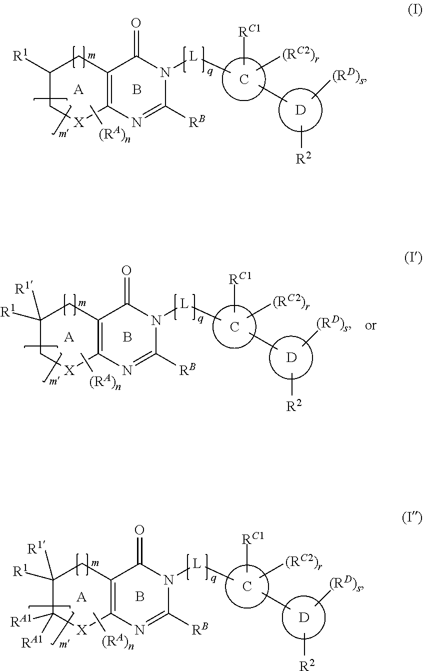

or pharmaceutically acceptable salts, solvates, stereoisomers, or prodrugs thereof, wherein each of the variables in Formulae I and I′ is described, embodied, and exemplified herein.

In certain aspects, the present disclosure provides pharmaceutical compositions comprising the compound disclosed herein and a pharmaceutically acceptable excipient.

In certain aspects, the present disclosure provides methods of inhibiting a protein in a subject or biological sample comprising administering the compound disclosed herein to the subject or contacting the biological sample with the compound disclosed herein.

In certain aspects, the present disclosure provides uses of the compound disclosed herein in the manufacture of a medicament for inhibiting a protein in a subject or biological sample.

In certain aspects, the present disclosure provides compounds disclosed herein for use in inhibiting a protein in a subject or biological sample.

In certain aspects, the present disclosure provides methods of treating or preventing a disease or disorder a subject in need thereof, comprising administering to the subject the compound disclosed herein.

In certain aspects, the present disclosure provides uses of the compound disclosed herein in the manufacture of a medicament for treating or preventing a disease or disorder in a subject in need thereof.

In certain aspects, the present disclosure provides compounds disclosed herein for use in treating or preventing a disease or disorder in a subject in need thereof.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

All publications and patents mentioned herein are hereby incorporated by reference in their entirety as if each individual publication or patent is specifically and individually indicated to be incorporated by reference. In case of conflict, the present application, including any definitions herein, will control.

### DETAILED DESCRIPTION

The present disclosure relates to compounds that inhibit eIF4E activity, as well as pharmaceutical compositions thereof. The present disclosure further relates to methods of inhibiting a protein in a subject or biological sample comprising administering a compound described herein to the subject or contacting the biological sample with a compound described herein. The present disclosure also relates to methods of treating or preventing a disease or disorder a subject in need thereof, comprising administering to the subject a compound described herein.

### Compounds of the Application

In certain aspects, the present disclosure provides compounds of Formula I″:

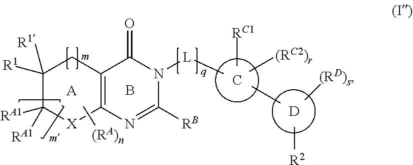

or a pharmaceutically acceptable salt, solvate, stereoisomer, or prodrug thereof,
wherein:
each -L- is independently —O—, —NR^{L}—, —CR^{L1}R^{L2}—, —CR^{L1}═CR^{L2}—, or —C≡C—;
each R^{L} is independently hydrogen or optionally substituted C₁₋₆ alkyl;
each R^{L1} and each R^{L2} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted;
q is an integer selected from 1 to 5;
Ring C and Ring D are independently C₆₋₁₀ aryl or 5- to 10-membered heteroaryl;
R^{C1}, each R^{C2} and each R^{D} are independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
r and s are independently an integer selected from 0 to 6, as valency permits;
R² is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —C(═O)NR^{c}S(═O)₂R^{a}, —C(═O)NR^{c}R^{d}, —(CH₂)C(═O)OR, or —C(═O)OR^{b}, wherein the alkyl, alkenyl, alkynyl, alkoxy, alkylamino, carbocyclyl, heterocyclyl, aryl, or heteroaryl is optionally substituted,
R¹ is —NR^{1a}R^{1b} or —OR^{1c};
R^{1′} is hydrogen, deuterium, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
R¹ and R^{1′}, together with the carbon atom to which they are bonded, from C₃₋₁₂ carbocyclyl or 3- to 12-membered heterocyclyl, wherein the carbocyclyl or heterocyclyl is optionally substituted;
R^{1a} and R^{1b} are each independently hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted; or
R^{1a} and R^{1b}, together with the nitrogen atom to which they are bonded, form 3- to 12-membered heterocycle, wherein the heterocycle is optionally substituted with one or more R^{ab};
each R^{ab} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, 3- to 6-membered heterocyclyl, C₆₋₁₀ aryl, 5- to 10-membered heteroaryl, or —S(═O)R^{a}, wherein the alkyl, alkenyl, alkynyl, alkoxy, alkylamino, carbocyclyl, heterocyclyl, aryl, or heteroaryl is optionally substituted; or
two vicinal R^{ab}, together with the atoms to which they are bonded, form C₆ aryl or 5- to 6-membered heteroaryl, wherein the aryl or heteroaryl is optionally substituted;
R^{1c} is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted;
X is —O— or —C(R^{X})₂—;
each R^{X} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
two R^{X}, together with the carbon atom to which they are bonded, form an oxo; or
two R^{X}, together with the carbon atom to which they are bonded, form C₃₋₆ carbocyclyl, or 3- to 6-membered heterocyclyl, wherein the carbocyclyl or heterocyclyl is optionally substituted;
each R^{A1} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
R^{X} and a vincinal R^{A1}, together with the carbon atoms to which they are bonded, form C₃₋₄ carbocyclyl, or 3- to 4-membered heterocyclyl, wherein the carbocyclyl or heterocyclyl is optionally substituted;
m and m′ are independently an integer selected from 0 to 2;
each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
n is an integer selected from 0 to 10, as valency permits;
R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
   wherein:
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and each R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are bonded, form 3- to 12-membered heterocyclyl;
wherein each occurrence of R^{a}, R^{b}, R^{c}, and R^{d} is independently and optionally substituted.

In certain embodiments, the present disclosure provides compounds of Formula I′:

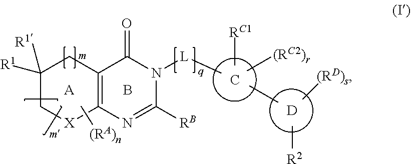

or pharmaceutically acceptable salts, solvates, stereoisomers, or prodrugs thereof,
wherein:
each -L- is independently —O—, —NR^{L}—, —CR^{L1}R^{L2}—, —CR^{L1}═CR^{L2}—, or —C≡C—
each R^{L} is independently hydrogen or optionally substituted C₁₋₆ alkyl;
each R^{L1} and each R^{L2} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted;
q is an integer selected from 1 to 5;
Ring C and Ring D are independently C₆₋₁₀ aryl or 5- to 10-membered heteroaryl;
R^{C1}, each R^{C2} and each R^{D} are independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
r and s are independently an integer selected from 0 to 6, as valency permits;
R² is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —C(═O)NR^{c}S(═O)₂R^{a}, —C(═O)NR^{c}R^{d}, —(CH₂)C(═O)OR, or —C(═O)OR^{b}, wherein the alkyl, alkenyl, alkynyl, alkoxy, alkylamino, carbocyclyl, heterocyclyl, aryl, or heteroaryl is optionally substituted,
R¹ is —NR^{1a}R^{1b} or —OR^{1c};
R^{1′} is hydrogen, deuterium, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
R¹ and R^{1′}, together with the carbon atom to which they are bonded, from C₃₋₁₂ carbocyclyl or 3- to 12-membered heterocyclyl, wherein the carbocyclyl or heterocyclyl is optionally substituted;
R^{1a} and R^{1b} are each independently hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted; or
R^{1a} and R^{1b}, together with the nitrogen atom to which they are bonded, form 3- to 12-membered heterocycle, wherein the heterocycle is optionally substituted with one or more R^{ab};
each R^{ab} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, 3- to 6-membered heterocyclyl, C₆₋₁₀ aryl, 5- to 10-membered heteroaryl, or —S(═O)R^{a}, wherein the alkyl, alkenyl, alkynyl, alkoxy, alkylamino, carbocyclyl, heterocyclyl, aryl, or heteroaryl is optionally substituted; or
two vicinal R^{a}b, together with the atoms to which they are bonded, form C₆ aryl or 5- to 6-membered heteroaryl, wherein the aryl or heteroaryl is optionally substituted;
R^{1c} is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted;
X is —O— or —C(R^{X})₂—;
each R^{X} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
two R^{X}, together with the carbon atom to which they are bonded, form an oxo; or
two R^{X}, together with the carbon atom to which they are bonded, form C₃₋₆ carbocyclyl, or 3- to 6-membered heterocyclyl, wherein the carbocyclyl or heterocyclyl is optionally substituted;
m and m′ are independently an integer selected from 0 to 2;
each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
n is an integer selected from 0 to 10, as valency permits;
R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
   wherein:
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and each R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are bonded, form 3- to 12-membered heterocyclyl;
   wherein each occurrence of R^{a}, R^{b}, R^{c}, and R^{d} is independently and optionally substituted.

In certain embodiments, the present disclosure provides a compound of Formula I:

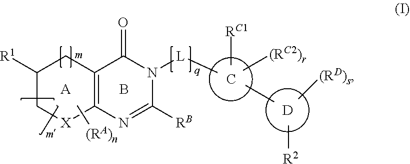

or a pharmaceutically acceptable salt, solvate, stereoisomer, or prodrug thereof,
wherein:
each -L- is independently —O—, —NR^{L}—, —CR^{L1}R^{L2}—, —CR^{L1}═CR^{L2}—, or —C≡C—;
each R^{L} is independently hydrogen or optionally substituted C₁₋₆ alkyl;
each R^{L1} and each R^{L2} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted;
q is an integer selected from 1 to 5;
Ring C and Ring D are independently C₆₋₁₀ aryl or 5- to 10-membered heteroaryl;
R^{C1}, each R^{C2}, and each R^{D} are independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
r and s are independently an integer selected from 0 to 6, as valency permits;
R² is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —C(═O)NR^{c}S(═O)₂R^{a}, or —C(═O)OR^{b}, wherein the alkyl, alkenyl, alkynyl, alkoxy, alkylamino, carbocyclyl, heterocyclyl, aryl, or heteroaryl is optionally substituted,
R¹ is —NR^{1a}R^{1b} or —OR^{1c};
R^{1a} and R^{1b} are each independently hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted; or
R^{1a} and R^{1b}, together with the nitrogen atom to which they are bonded, form 3- to 12-membered heterocycle, wherein the heterocycle is optionally substituted;
R^{1c} is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted;
X is —O— or —C(R^{X})₂—;
each R^{X} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
two geminal R^{X}, together with the carbon atom to which they are bonded, form an oxo;
m and m′ are independently an integer selected from 0 to 2;
each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
n is an integer selected from 0 to 10, as valency permits;
R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
   wherein:
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and each R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are bonded, form 3- to 12-membered heterocyclyl;
   wherein each occurrence of R^{a}, R^{b}, R^{c}, and R^{d} is independently and optionally substituted.

In certain embodiments, the compound of Formula I″ is a compound of Formula I″-1-i, I″-1-ii, I″-1-iii, or I″-1-iv:

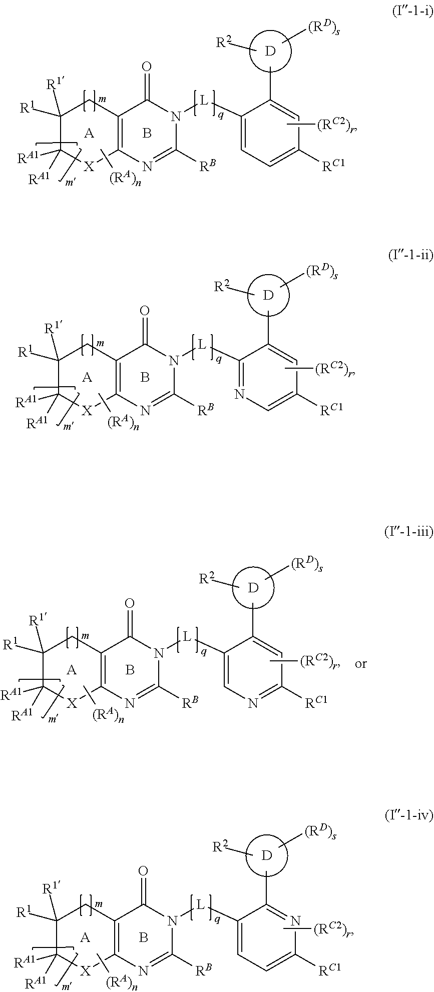

or a pharmaceutically acceptable salt, solvate, stereoisomer, or prodrug thereof.

In certain embodiments, the compound of Formula I′ is a compound of Formula I′-1-i, I′-1-ii, I′-1-iii, or I′-1-iv:

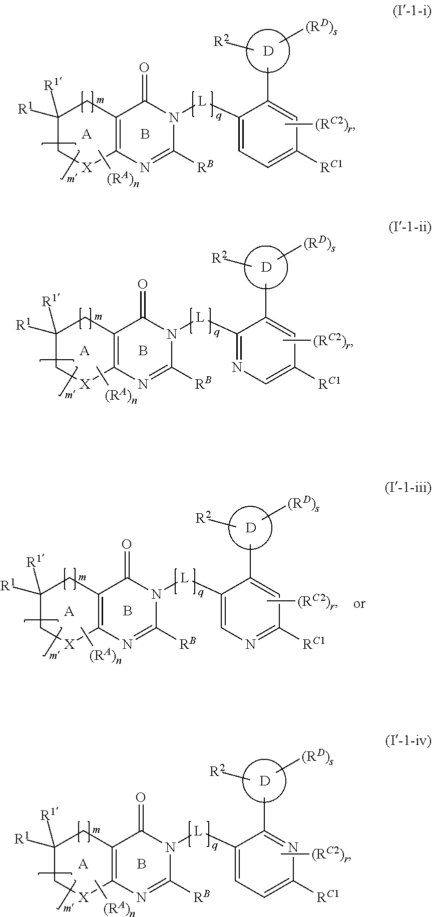

or a pharmaceutically acceptable salt, solvate, stereoisomer, or prodrug thereof.

In certain embodiments, the compound of Formula I is a compound of Formula I-1-i, I-1-ii, I-1-iii, or I-1-iv:

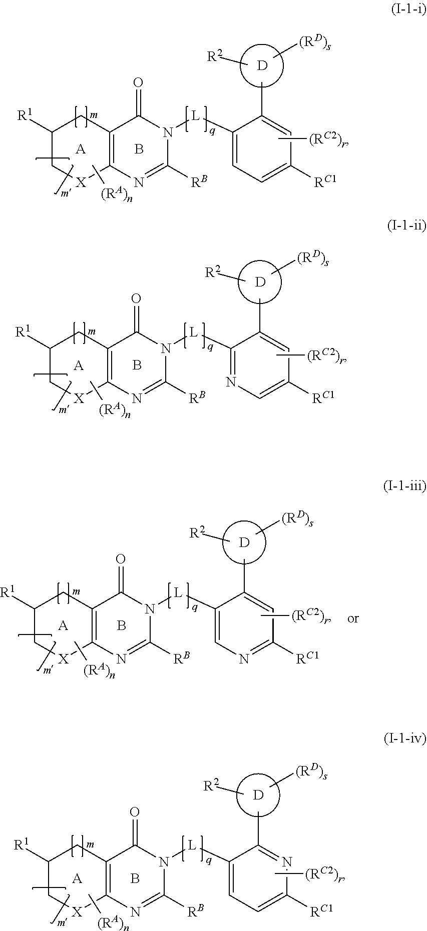

or a pharmaceutically acceptable salt, solvate, stereoisomer, or prodrug thereof.

In certain embodiments, the compound of Formula I″ is a compound of Formula I″-1-i-1, I″-1-i-2, I″-1-i-3, I″-1-iii-1, I″-1-iii-2, or I″-1-iii-3:

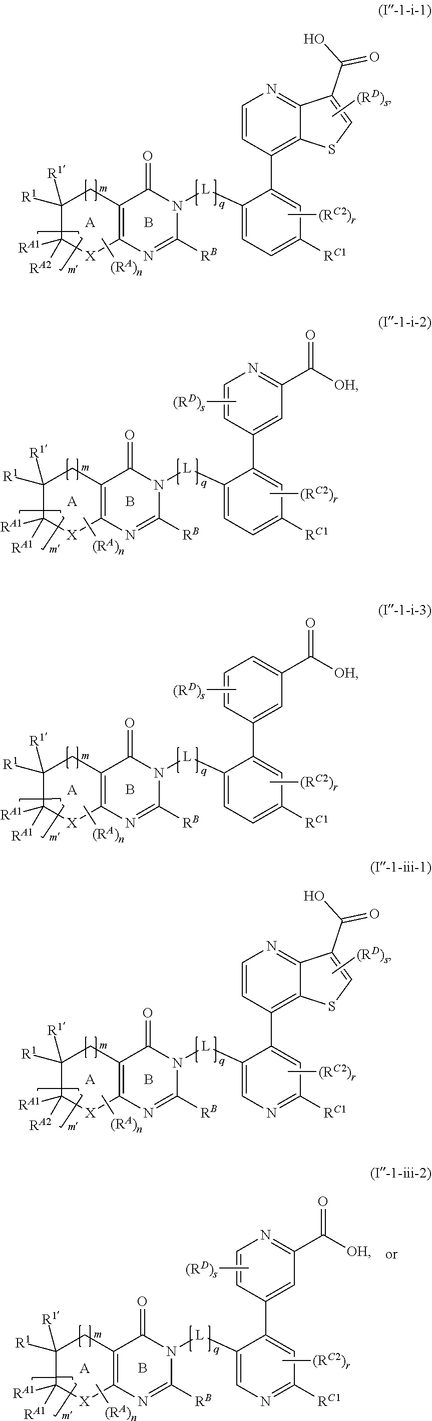

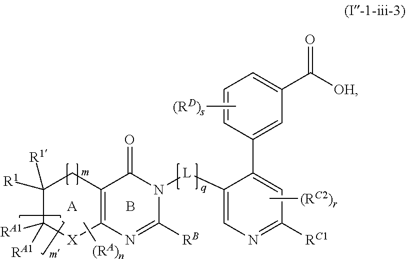

or a pharmaceutically acceptable salt, solvate, stereoisomer, or prodrug thereof.

In certain embodiments, the compound of Formula I′ is a compound of Formula I′-1-i-1, I′-1-i-2, I′-1-i-3, I′-1-iii-1, I′-1-iii-2, or I′-1-iii-3:

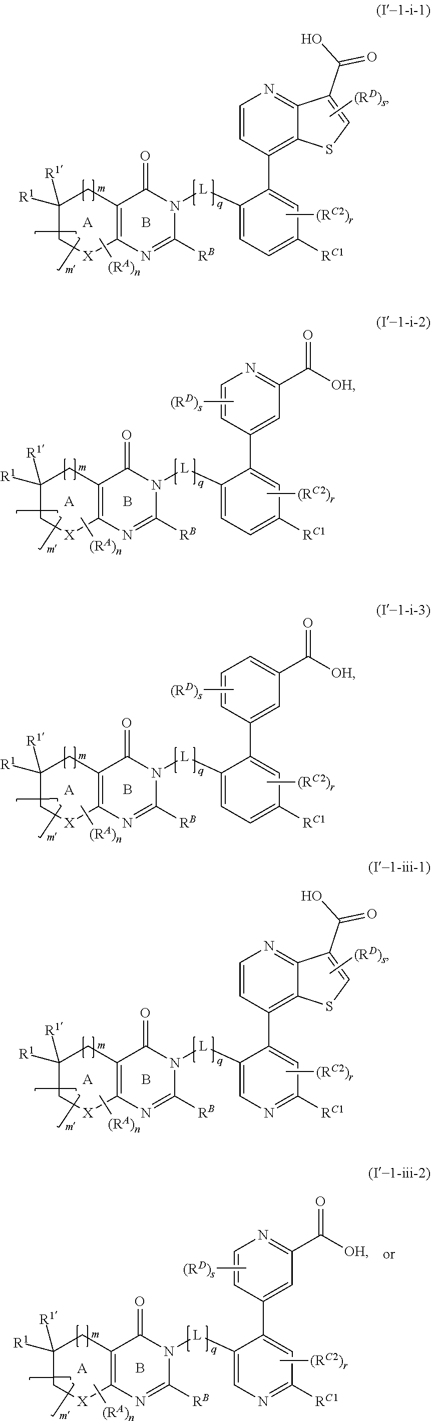

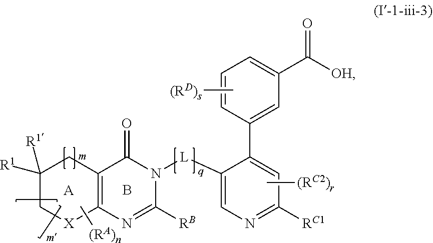

or a pharmaceutically acceptable salt, solvate, stereoisomer, or prodrug thereof.

In certain embodiments, the compound of Formula I is a compound of Formula I-1-i-1, I-1-i-2, I-1-i-3, I-1-iii-1, I-1-iii-2, or I-1-iii-3:

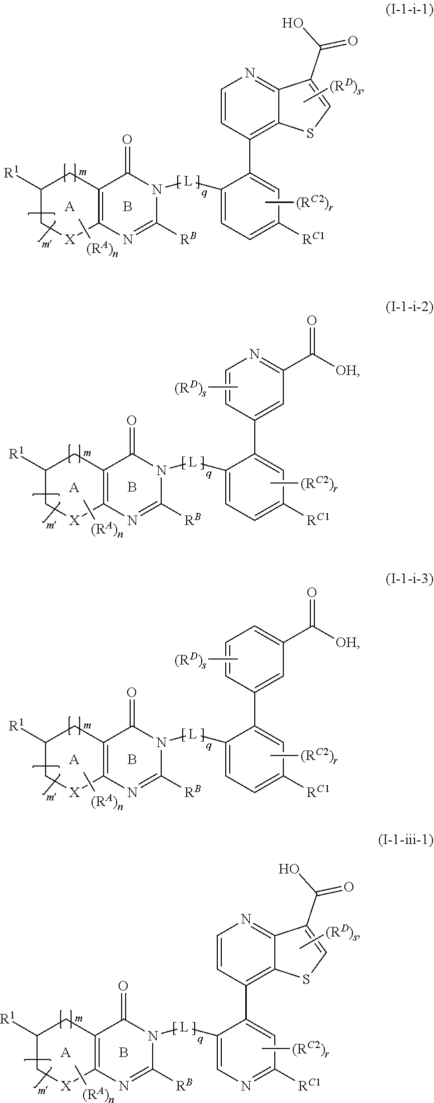

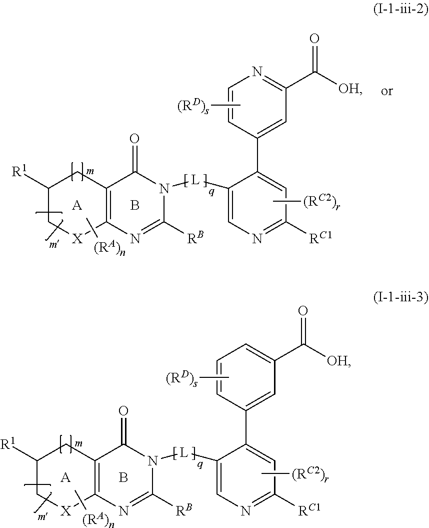

or a pharmaceutically acceptable salt, solvate, stereoisomer, or prodrug thereof.

Embodiments of the variables in any of the Formulae described herein, e.g., Formula I-Formula I-1-iii-3, as applicable, are described below. Any of the variables can be any moiety as described in the embodiments below. Also, any moieties described for any of the variables can be combined, as applicable, with any moieties described for any of the remaining variables.

In certain embodiments, R¹ is —NR^{1a}R^{1b} or —OR^{1c}. In certain embodiments, R¹ is —NR^{1a}R^{1b}.

In certain embodiments, R¹ is —OR^{1c}.

In certain embodiments, R¹ is —NR^{1a}R^{1b}.

In certain embodiments, R^{1a} and R^{1b} are each independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₁₂ carbocyclyl (e.g., monocyclic or polycyclic (e.g., sprio, bridged, or fused) carbocyclyl including, e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., monocyclic or polycyclic (e.g., sprio, bridged, or fused) heterocyclyl comprising one or two 3- to 8-membered rings and 1-5 heteroatoms selected from N, O, and S), —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl (e.g., monocyclic or polycyclic (e.g., sprio, bridged, or fused) carbocyclyl), —(C₁₋₆ alkylene)-(3- to 12-membered heterocyclyl (e.g., monocyclic or polycyclic (e.g., sprio, bridged, or fused) heterocyclyl), —S(═O)R^{a}, —S(═O)₂R^{a}, —S(═O)₂OR^{b}, —S(═O)₂NR^{c}R^{d}, —C(═O)R^{a}, —C(═O)R^{b}, or —C(═O)NR^{c}R^{d}, wherein the alkyl, alkenyl, alkynyl, alkylene, carbocyclyl, heterocyclyl, aryl, or heteroaryl is optionally substituted. In certain embodiments, the C₁₋₆ alkene is selected from is selected from methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—). In certain embodiments, R^{1a} and R^{1b} are each optionally independently substituted with one or more R^{u}.

In certain embodiments, R^{1a} and R^{1b} are each independently hydrogen, C₁₋₆ alkyl, C₆₋₁₀ aryl, 5- to 10-membered heteroaryl, C₃₋₁₂ carbocyclyl (e.g., monocyclic or polycyclic (e.g., sprio, bridged, or fused) carbocyclyl), 3- to 12-membered heterocyclyl (e.g., monocyclic or polycyclic (e.g., sprio, bridged, or fused) heterocyclyl), —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl (e.g., monocyclic or polycyclic (e.g., sprio, bridged, or fused) carbocyclyl)), —(C₁₋₆ alkylene)-(3- to 12-membered heterocyclyl (e.g., monocyclic or polycyclic (e.g., sprio, bridged, or fused) heterocyclyl)), —S(═O)R^{a}, —S(═O)₂R^{a}, —S(═O)₂OR^{b}, —S(═O)₂NR^{c}R^{d}, —C(═O)R^{a}, —C(═O)R^{b}, or —C(═O)NR^{c}R^{d}, wherein the alkyl, alkenyl, alkynyl, alkylene, carbocyclyl, heterocyclyl, aryl, or heteroaryl is optionally substituted. In certain embodiments, R^{1a} and R^{1b} are each optionally independently substituted with one or more R^{u}.

In certain embodiments, R^{1a} and R^{1b} are each independently hydrogen, C₁₋₆ alkyl, C₆₋₁₀ aryl, 5- to 10-membered heteroaryl, C₃₋₁₂ carbocyclyl (e.g., monocyclic or polycyclic (e.g., sprio, bridged, or fused) carbocyclyl), 3- to 12-membered heterocyclyl (e.g., monocyclic or polycyclic (e.g., sprio, bridged, or fused) heterocyclyl), —(C₁₋₆ alkylene)-(5- to 10-membered heteroaryl), —(C₁₋₆ alkylene)-(C₆₋₁₀ aryl), —(C₁₋₆ alkylene)-(C₃₋₁₂ carbocyclyl (e.g., monocyclic or polycyclic (e.g., sprio, bridged, or fused) carbocyclyl)), —(C₁₋₆ alkylene)-(3- to 12-membered heterocyclyl (e.g., monocyclic or polycyclic (e.g., sprio, bridged, or fused) heterocyclyl)), —S(═O)₂R^{a}, or —C(═O)R^{a}, wherein the alkyl, alkylene, carbocyclyl, heterocyclyl, or heteroaryl is optionally substituted. In certain embodiments, R^{1a} and R^{1b} are each optionally independently substituted with one or more R^{u}.

In certain embodiments, R^{1a} and R^{1b} are each independently hydrogen, —CN, C₁₋₆ alkyl, C₃₋₁₂ carbocyclyl (e.g., monocyclic or polycyclic (e.g., sprio, bridged, or fused) carbocyclyl), 3- to 12-membered heterocyclyl (e.g., monocyclic or polycyclic (e.g., sprio, bridged, or fused) heterocyclyl), —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl (e.g., monocyclic or polycyclic (e.g., sprio, bridged, or fused) carbocyclyl)), or —(C₁₋₆ alkylene)-(3- to 12-membered heterocyclyl (e.g., monocyclic or polycyclic (e.g., sprio, bridged, or fused) heterocyclyl)), wherein the alkyl, alkylene, carbocyclyl, heterocyclyl, aryl, or heteroaryl is optionally substituted.

In certain embodiments, R^{1a} and R^{1b} are each independently hydrogen, optionally substituted C₁₋₆ alkyl, —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}.

In certain embodiments, at least one of R^{1a} and R^{1b} is hydrogen. at least one of R^{1a} and R^{1b} is not hydrogen. In certain embodiments, at least one of R^{1a} and R^{1b} is optionally substituted C₁₋₆ alkyl. In certain embodiments, at least one of R^{1a} and R^{1b} is optionally substituted C₆₋₁₀ aryl.

In certain embodiments, at least one of R^{1a} and R^{1b} is optionally substituted 5- to 10-membered heteroaryl comprising one or two 5- or 6-membered rings and 1-5 heteroatoms selected from N, O, and S. In certain embodiments, at least one of R^{1a} and R^{1b} is optionally substituted C₃₋₁₂ carbocyclyl (e.g., monocyclic or polycyclic (e.g., sprio, bridged, or fused) carbocyclyl). In certain embodiments, at least one of R^{1a} and R^{1b} is optionally substituted 3- to 12-membered heterocyclyl (e.g., monocyclic or polycyclic (e.g., sprio, bridged, or fused) heterocyclyl) comprising one or two 3- to 8-membered rings and 1-5 heteroatoms selected from N, O, and S. In certain embodiments, at least one of R^{1a} and R^{1b} is —(C₁₋₆ alkylene)-(C₆₋₁₀ aryl), wherein the alkylene or aryl is optionally substituted. In certain embodiments, at least one of R^{1a} and R^{1b} is —(C₁₋₆ alkylene)-(5- to 10-membered heteroaryl), wherein the heteroaryl comprises one or two 5- or 6-membered rings and 1-5 heteroatoms selected from N, O, and S, wherein the alkylene or heteroaryl is optionally substituted. In certain embodiments, at least one of R^{1a} and R^{1b} is —(C₁₋₆ alkylene)-(C₃₋₁₂ carbocyclyl (e.g., monocyclic or polycyclic (e.g., sprio, bridged, or fused) carbocyclyl)), wherein the alkylene or carbocyclyl is optionally substituted. In certain embodiments, at least one of R^{1a} and R^{1b} is —(C₁₋₆ alkylene)-(3- to 12-membered heterocyclyl (e.g., monocyclic or polycyclic (e.g., sprio, bridged, or fused) heterocyclyl)), wherein the heterocyclyl comprises one or two 3- to 8-membered rings and 1-5 heteroatoms selected from N, O, and S, wherein the alkylene or heterocyclyl is optionally substituted. In certain embodiments, at least one of R^{1a} and R^{1b} is —S(═O)₂R^{a}. In certain embodiments, at least one of R^{1a} and R^{1b} is —C(═O)R^{a}. In certain embodiments, R^{1a} and R^{1b} is optionally substituted with one or more R^{u}.

In certain embodiments, at least one of R^{1a} and R^{1b} is optionally substituted C₆₋₁₀ aryl. In certain embodiments, the aryl is optionally substituted with one or more R^{u}.

In certain embodiments, at least one of R^{1a} and R^{1b} is optionally substituted 5- to 10-membered heteroaryl comprising one or two 5- or 6-membered rings and 1-5 heteroatoms selected from N, O, and S. In certain embodiments, at least one of R^{1a} and R^{1b} is optionally substituted heteroaryl comprising one 5- or 6-membered ring and 1-3 heteroatoms selected from N, O, and S. In certain embodiments, at least one of R^{1a} and R^{1b} is optionally substituted heteroaryl comprising one 5-membered ring and 1-3 heteroatoms selected from N, O, and S. In certain embodiments, at least one of R^{1a} and R^{1b} is optionally substituted heteroaryl comprising one 6-membered ring and 1-3 heteroatoms selected from N, O, and S. In certain embodiments, at least one of R^{1a} and R^{1b} is optionally substituted heteroaryl comprising two 5- or 6-membered rings and 1-5 heteroatoms selected from N, O, and S. In certain embodiments, at least one of R^{1a} and R^{1b} is optionally substituted heteroaryl comprising two 5-membered rings and 1-5 heteroatoms selected from N, O, and S. In certain embodiments, at least one of R^{1a} and R^{1b} is optionally substituted heteroaryl comprising two 6-membered rings and 1-5 heteroatoms selected from N, O, and S. In certain embodiments, at least one of R^{1a} and R^{1b} is optionally substituted heteroaryl comprising one 5-membered ring and one 6-membered ring and 1-5 heteroatoms selected from N, O, and S. In certain embodiments, the heteroaryl is optionally substituted with one or more R^{u}.

In certain embodiments, at least one of R^{1a} and R^{1b} is optionally substituted C₃₋₁₂ carbocyclyl (e.g., monocyclic or polycyclic (e.g., sprio, bridged, or fused) carbocyclyl including, e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)). In certain embodiments, the carbocyclyl is optionally substituted with one or more R^{u}.

In certain embodiments, at least one of R^{1a} and R^{1b} is optionally substituted 3- to 12-membered heterocyclyl (e.g., monocyclic or polycyclic (e.g., sprio, bridged, or fused) heterocyclyl) comprising one or two 3- to 8-membered rings and 1-5 heteroatoms selected from N, O, and S. In certain embodiments, at least one of R^{1a} and R^{1b} is optionally substituted heterocyclyl comprising one 3- to 8-membered ring and 1-3 heteroatoms selected from N, O, and S. In certain embodiments, at least one of R^{1a} and R^{1b} is optionally substituted heterocyclyl comprising one 3-membered ring and 1-3 heteroatoms selected from N, O, and S. In certain embodiments, at least one of R^{1a} and R^{1b} is optionally substituted heterocyclyl comprising one 4-membered ring and 1-3 heteroatoms selected from N, O, and S. In certain embodiments, at least one of R^{1a} and R^{1b} is optionally substituted heterocyclyl comprising one 5-membered ring and 1-3 heteroatoms selected from N, O, and S. In certain embodiments, at least one of R^{1a} and R^{1b} is optionally substituted heterocyclyl comprising one 6-membered ring and 1-4 heteroatoms selected from N, O, and S. In certain embodiments, at least one of R^{1a} and R^{1b} is optionally substituted heterocyclyl comprising one 7-membered ring and 1-4 heteroatoms selected from N, O, and S. In certain embodiments, at least one of R^{1a} and R^{1b} is optionally substituted heterocyclyl comprising one 8-membered ring and 1-5 heteroatoms selected from N, O, and S. In certain embodiments, at least one of R^{1a} and R^{1b} is optionally substituted heterocyclyl (e.g., polycyclic (e.g., sprio, bridged, or fused) heterocyclyl) comprising two 3- to 8-membered rings and 1-5 heteroatoms selected from N, O, and S. In certain embodiments, at least one of R^{1a} and R^{1b} is optionally substituted heterocyclyl (e.g., polycyclic (e.g., sprio, bridged, or fused) heterocyclyl) comprising two 5-membered rings and 1-5 heteroatoms selected from N, O, and S. In certain embodiments, at least one of R^{1a} and R^{1b} is optionally substituted heterocyclyl (e.g., polycyclic (e.g., sprio, bridged, or fused) heterocyclyl) comprising two 6-membered rings and 1-5 heteroatoms selected from N, O, and S. In certain embodiments, at least one of R^{1a} and R^{1b} is optionally substituted heterocyclyl (e.g., polycyclic (e.g., sprio, bridged, or fused) heterocyclyl) comprising one 5-membered ring and one 6-membered ring and 1-5 heteroatoms selected from N, O, and S. In certain embodiments, the heterocyclyl is optionally substituted with one or more R^{u}.

In certain embodiments, at least one of R^{1a} and R^{1b} is —(C₁₋₆ alkylene)-(C₆₋₁₀ aryl), wherein the alkylene or aryl is optionally substituted. In certain embodiments, the aryl is optionally substituted with one or more R^{u}.

In certain embodiments, at least one of R^{1a} and R^{1b} is —(C₁₋₆ alkylene)-(5- to 10-membered heteroaryl), wherein the heteroaryl comprises one or two 5- or 6-membered rings and 1-5 heteroatoms selected from N, O, and S, wherein the alkylene or heteroaryl is optionally substituted. In certain embodiments, the optionally substituted heteroaryl comprises one 5- or 6-membered ring and 1-3 heteroatoms selected from N, O, and S. In certain embodiments, the optionally substituted heteroaryl comprises one 5-membered ring and 1-3 heteroatoms selected from N, O, and S. In certain embodiments, the optionally substituted heteroaryl comprises one 6-membered ring and 1-3 heteroatoms selected from N, O, and S. In certain embodiments, the optionally substituted heteroaryl comprises two 5- or 6-membered rings and 1-5 heteroatoms selected from N, O, and S. In certain embodiments, the optionally substituted heteroaryl comprises two 5-membered rings and 1-5 heteroatoms selected from N, O, and S. In certain embodiments, the optionally substituted heteroaryl comprises two 6-membered rings and 1-5 heteroatoms selected from N, O, and S. In certain embodiments, the optionally substituted heteroaryl comprises one 5-membered ring and one 6-membered ring and 1-5 heteroatoms selected from N, O, and S. In certain embodiments, the heteroaryl is optionally substituted with one or more R^{u}.

In certain embodiments, at least one of R^{1a} and R^{1b} is —(C₁₋₆ alkylene)-(C₃₋₁₂ carbocyclyl (e.g., monocyclic or polycyclic (e.g., sprio, bridged, or fused) carbocyclyl), wherein the C₃₋₁₂ carbocyclyl, e.g., monocyclic or polycyclic (e.g., sprio, bridged, or fused) carbocyclyl, includes cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), and spiro[4.5]decanyl (C₁₀), wherein the alkylene or carbocyclyl is optionally substituted. In certain embodiments, the carbocyclyl is optionally substituted with one or more R^{u}.

In certain embodiments, at least one of R^{1a} and R^{1b} is —(C₁₋₆ alkylene)-(3- to 12-membered heterocyclyl (e.g., monocyclic or polycyclic (e.g., sprio, bridged, or fused) heterocyclyl)), wherein the heterocyclyl (e.g., monocyclic or polycyclic (e.g., sprio, bridged, or fused) heterocyclyl) comprises one or two 3- to 8-membered rings and 1-5 heteroatoms selected from N, O, and S, wherein the alkylene or heterocyclyl is optionally substituted. In certain embodiments, the optionally substituted heterocyclyl comprises one 3- to 8-membered ring and 1-3 heteroatoms selected from N, O, and S. In certain embodiments, the optionally substituted heterocyclyl comprises one 3-membered ring and 1-3 heteroatoms selected from N, O, and S. In certain embodiments, the optionally substituted heterocyclyl comprises one 4-membered ring and 1-3 heteroatoms selected from N, O, and S. In certain embodiments, the optionally substituted heterocyclyl comprises one 5-membered ring and 1-3 heteroatoms selected from N, O, and S. In certain embodiments, the optionally substituted heterocyclyl comprises one 6-membered ring and 1-4 heteroatoms selected from N, O, and S. In certain embodiments, the optionally substituted heterocyclyl comprises one 7-membered ring and 1-4 heteroatoms selected from N, O, and S. In certain embodiments, the optionally substituted heterocyclyl comprises one 8-membered ring and 1-5 heteroatoms selected from N, O, and S. In certain embodiments, the optionally substituted heterocyclyl (e.g., polycyclic (e.g., sprio, bridged, or fused) heterocyclyl) comprises two 3- to 8-membered rings and 1-5 heteroatoms selected from N, O, and S. In certain embodiments, the optionally substituted heterocyclyl (e.g., polycyclic (e.g., sprio, bridged, or fused) heterocyclyl) comprises two 5-membered rings and 1-5 heteroatoms selected from N, O, and S. In certain embodiments, the optionally substituted heterocyclyl (e.g., polycyclic (e.g., sprio, bridged, or fused) heterocyclyl) comprises two 6-membered rings and 1-5 heteroatoms selected from N, O, and S. In certain embodiments, the optionally substituted heterocyclyl (e.g., polycyclic (e.g., sprio, bridged, or fused) heterocyclyl) comprises one 5-membered ring and one 6-membered ring and 1-5 heteroatoms selected from N, O, and S. In certain embodiments, the heterocyclyl is optionally substituted with one or more R^{u}.

In certain embodiments, at least one of R^{1a} and R^{1b} is —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}. In certain embodiments, at least one of R^{1a} and R^{1b} is —S(═O)₂R^{a}. In certain embodiments, at least one of R^{1a} and R^{1b} is —C(═O)R^{a}.

In certain embodiments, R^{1a} and R^{1b}, together with the nitrogen atom to which they are bonded, form optionally substituted 3- to 12-membered heterocycle (e.g., monocyclic or polycylic (e.g., sprio, bridged, or fused) heterocycle comprising one or two 3- to 8-membered rings and 1-5 heteroatoms selected from N, O, and S). In certain embodiments, R^{1a} and R^{1b}, together with the nitrogen atom to which they are bonded, form 3- to 12-membered heterocycle optionally substituted with one or more R^{u}. In certain embodiments, R^{1a} and R^{1b}, together with the nitrogen atom to which they are bonded, form 3- to 12-membered heterocycle optionally substituted with one or more R^{ab}. In certain embodiments, the optionally substituted heterocyclyl comprises one 3- to 8-membered ring and 1-3 heteroatoms selected from N, O, and S. In certain embodiments, the optionally substituted heterocyclyl comprises one 3-membered ring and 1-3 heteroatoms selected from N, O, and S. In certain embodiments, the optionally substituted heterocyclyl comprises one 4-membered ring and 1-3 heteroatoms selected from N, O, and S. In certain embodiments, the optionally substituted heterocyclyl comprises one 5-membered ring and 1-3 heteroatoms selected from N, O, and S. In certain embodiments, the optionally substituted heterocyclyl comprises one 6-membered ring and 1-4 heteroatoms selected from N, O, and S. In certain embodiments, the optionally substituted heterocyclyl comprises one 7-membered ring and 1-4 heteroatoms selected from N, O, and S. In certain embodiments, the optionally substituted heterocyclyl comprises one 8-membered ring and 1-5 heteroatoms selected from N, O, and S. In certain embodiments, the optionally substituted heterocyclyl (e.g., polycyclic (e.g., sprio, bridged, or fused) heterocyclyl) comprises two 3- to 8-membered rings and 1-5 heteroatoms selected from N, O, and S. In certain embodiments, the optionally substituted heterocyclyl (e.g., polycyclic (e.g., sprio, bridged, or fused) heterocyclyl) comprises two 5-membered rings and 1-5 heteroatoms selected from N, O, and S. In certain embodiments, the optionally substituted heterocyclyl (e.g., polycyclic (e.g., sprio, bridged, or fused) heterocyclyl) comprises two 6-membered rings and 1-5 heteroatoms selected from N, O, and S. In certain embodiments, the optionally substituted heterocyclyl (e.g., polycyclic (e.g., sprio, bridged, or fused) heterocyclyl) comprises one 5-membered ring and one 6-membered ring and 1-5 heteroatoms selected from N, O, and S.

In certain embodiments, each R^{ab} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), C₆₋₁₀ aryl (i.e., phenyl or naphathalenyl), or 5- to 10-membered heteroaryl (e.g., heteroaryl comprising one or two rings and 1-5 heteroatoms selected from N. O, and S), wherein the alkyl, alkenyl, alkynyl, alkoxy, alkylamino, carbocyclyl, heterocyclyl, aryl, or heteroaryl is optionally substituted. In certain embodiments, R^{ab} is optionally substituted with one or more R^{u}.

In certain embodiments, each R^{ab} is independently oxo, halogen, —OH, C₁₋₆ alkyl, C₁₋₆ alkoxy, C₃₋₆ carbocyclyl, 3- to 6-membered heterocyclyl, C₆ aryl, or 5- to 6-membered heteroaryl, or —S(═O)R^{a}, wherein the alkyl, alkenyl, alkynyl, alkoxy, alkylamino, carbocyclyl, heterocyclyl, aryl, or heteroaryl is optionally substituted with one or more R^{u}.

In certain embodiments, each R^{ab} is independently oxo, halogen, —OH, C₁₋₆ alkyl, C₁₋₆ alkoxy, C₃₋₆ carbocyclyl, 3- to 6-membered heterocyclyl, C₆₋₁₀ aryl, 5- to 10-membered heteroaryl, or —S(═O)R^{a}, wherein the alkyl, alkenyl, alkynyl, alkoxy, alkylamino, carbocyclyl, heterocyclyl, aryl, or heteroaryl is optionally substituted with one or more R^{u}.

In certain embodiments, two vicinal R^{ab}, together with the atoms to which they are bonded, form C₆ aryl (i.e., phenyl) or 5- to 6-membered heteroaryl (e.g., heteroaryl comprising one 5- or 6-membered rings and 1-4 heteroatoms selected from N, O, and S), wherein the aryl or heteroaryl is optionally substituted. In certain embodiments, two vicinal R^{ab}, together with the atoms to which they are bonded, form C₆ aryl or 5- to 6-membered heteroaryl, wherein the aryl or heteroaryl is optionally substituted with one or more R^{u}.

In certain embodiments, R¹ is —OR^{1c}.

In certain embodiments, R^{1c} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), —C(═O)R^{a}, —C(═O)OR^{b}, or —C(═O)NR^{c}R^{d}, wherein the alkyl, carbocyclyl, heterocyclyl, aryl, or heteroaryl is optionally substituted. In certain embodiments, R^{1c} is optionally substituted with one or more R^{u}.

In certain embodiments, R^{1c} is hydrogen, C₁₋₆ alkyl, C₆₋₁₀ aryl, 5- to 10-membered heteroaryl, C₃₋₁₂ carbocyclyl, 3- to 12-membered heterocyclyl, —C(═O)R^{a}, —C(═O)OR, or —C(═O)NR^{c}R^{d}, wherein the alkyl, carbocyclyl, heterocyclyl, aryl, or heteroaryl is optionally substituted. In certain embodiments, R^{1c} is optionally substituted with one or more R^{u}.

In certain embodiments, R^{1c} is C₁₋₆ alkyl, C₆₋₁₀ aryl, 5- to 10-membered heteroaryl, C₃₋₁₂ carbocyclyl, or 3- to 12-membered heterocyclyl, wherein the alkyl, carbocyclyl, heterocyclyl, aryl, or heteroaryl is optionally substituted. In certain embodiments, R^{1c} is optionally substituted with one or more R^{u}.

In certain embodiments, R^{1c} is C₁₋₆ alkyl or 3- to 12-membered heterocyclyl, the alkyl or heterocyclyl is optionally substituted. In certain embodiments, R^{1c} is optionally substituted C₁₋₆ alkyl. In certain embodiments, R^{1c} is optionally substituted 3- to 12-membered heterocyclyl. In certain embodiments, R^{1c} is optionally substituted with one or more R^{u}.

In certain embodiments, R^{1c} is optionally substituted C₆₋₁₀ aryl. In certain embodiments, the aryl is optionally substituted with one or more R^{u}.

In certain embodiments, R^{1c} is optionally substituted 5- to 10-membered heteroaryl comprising one or two 5- or 6-membered rings and 1-5 heteroatoms selected from N, O, and S. In certain embodiments, R^{1c} is optionally substituted heteroaryl comprising one 5- or 6-membered ring and 1-3 heteroatoms selected from N, O, and S. In certain embodiments, R^{1c} is optionally substituted heteroaryl comprising one 5-membered ring and 1-3 heteroatoms selected from N, O, and S. In certain embodiments, R^{1c} is optionally substituted heteroaryl comprising one 6-membered ring and 1-3 heteroatoms selected from N, O, and S. In certain embodiments, R^{1c} is optionally substituted heteroaryl comprising two 5- or 6-membered rings and 1-5 heteroatoms selected from N, O, and S. In certain embodiments, R^{1c} is optionally substituted heteroaryl comprising two 5-membered rings and 1-5 heteroatoms selected from N, O, and S. In certain embodiments, R^{1c} is optionally substituted heteroaryl comprising two 6-membered rings and 1-5 heteroatoms selected from N, O, and S. In certain embodiments, R^{1c} is optionally substituted heteroaryl comprising one 5-membered ring and one 6-membered ring and 1-5 heteroatoms selected from N, O, and S. In certain embodiments, the heteroaryl is optionally substituted with one or more R^{u}.

In certain embodiments, R^{1c} is optionally substituted C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)). In certain embodiments, the carbocyclyl is optionally substituted with one or more R^{u}.

In certain embodiments, R^{1c} is optionally substituted 3- to 12-membered heterocyclyl comprising one or two 3- to 8-membered rings and 1-5 heteroatoms selected from N, O, and S. In certain embodiments, R^{1c} is optionally substituted heterocyclyl comprising one 3- to 8-membered ring and 1-3 heteroatoms selected from N, O, and S. In certain embodiments, R^{1c} is optionally substituted heterocyclyl comprising one 3-membered ring and 1-3 heteroatoms selected from N, O, and S. In certain embodiments, R^{1c} is optionally substituted heterocyclyl comprising one 4-membered ring and 1-3 heteroatoms selected from N, O, and S. In certain embodiments, R^{1c} is optionally substituted heterocyclyl comprising one 5-membered ring and 1-3 heteroatoms selected from N, O, and S. In certain embodiments, R^{1c} is optionally substituted heterocyclyl comprising one 6-membered ring and 1-4 heteroatoms selected from N, O, and S. In certain embodiments, R^{1c} is optionally substituted heterocyclyl comprising one 7-membered ring and 1-4 heteroatoms selected from N, O, and S. In certain embodiments, R^{1c} is optionally substituted heterocyclyl comprising one 8-membered ring and 1-5 heteroatoms selected from N, O, and S. In certain embodiments, R^{1c} is optionally substituted heterocyclyl comprising two 3- to 8-membered rings and 1-5 heteroatoms selected from N, O, and S. In certain embodiments, R^{1c} is optionally substituted heterocyclyl comprising two 5-membered rings and 1-5 heteroatoms selected from N, O, and S. In certain embodiments, R^{1c} is optionally substituted heterocyclyl comprising two 6-membered rings and 1-5 heteroatoms selected from N, O, and S. In certain embodiments, R^{1c} is optionally substituted heterocyclyl comprising one 5-membered ring and one 6-membered ring and 1-5 heteroatoms selected from N, O, and S. In certain embodiments, the heterocyclyl is optionally substituted with one or more R^{u}.

In certain embodiments, R^{1c} is hydrogen, deuterium, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, R^{1′} is optionally substituted with one or more R^{u}.

In certain embodiments, R^{1′} is hydrogen, deuterium, or optionally substituted C₁₋₆ alkyl.

In certain embodiments, R¹ and R^{1′}, together with the carbon atom to which they are bonded, from C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) or 3- to 12-membered heterocyclyl (e.g., heterocyclyl comprising one or two 3- to 8-membered rings and 1-5 heteroatoms selected from N, O, and S), wherein the carbocyclyl or heterocyclyl is optionally substituted with one or more R^{u}. In certain embodiments, R¹ and R^{1′}, together with the carbon atom to which they are bonded, from C₃₋₁₂ carbocyclyl or 3- to 12-membered heterocyclyl, wherein the carbocyclyl or heterocyclyl is optionally substituted with one or more R^{u}.

In certain embodiments, m is 0. In certain embodiments, m is 1. In certain embodiments, m is 2.

In certain embodiments, m′ is 0. In certain embodiments, m′ is 1. In certain embodiments, m′ is 2.

In certain embodiments, m is 0 and m′ is 0. In certain embodiments, m is 0 and m′ is 1. In certain embodiments, m is 0 and m′ is 2. In certain embodiments, m is 1 and m′ is 0. In certain embodiments, m is 1 and m′ is 1. In certain embodiments, m is 1 and m′ is 2. In certain embodiments, m is 2 and m′ is 0. In certain embodiments, m is 2 and m′ is 1. In certain embodiments, m is 2 and m′ is 2.

In certain embodiments, each R^{A} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{A} is independently optionally substituted with one or more R^{u}.

In certain embodiments, each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, or C₁₋₆ alkylamino, wherein the alkyl, alkenyl, alkynyl, alkoxy, or alkylamino is optionally substituted. In certain embodiments, each R^{A} is independently optionally substituted with one or more R^{u}.

In certain embodiments, each R^{A} is independently halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{A} is independently optionally substituted with one or more R^{u}.

In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, R^{B} is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-i-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, R^{B} is independently optionally substituted with one or more R^{u}.

In certain embodiments, R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, or C₁₋₆ alkylamino, wherein the alkyl, alkenyl, alkynyl, alkoxy, or alkylamino is optionally substituted. In certain embodiments, R^{B} is independently optionally substituted with one or more R^{u}.

In certain embodiments, R^{B} is hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, R^{B} is independently optionally substituted with one or more R^{u}.

In certain embodiments, R^{B} is hydrogen or optionally substituted C₁₋₆ alkyl. In certain embodiments, R^{B} is hydrogen. In certain embodiments, R^{B} is optionally substituted C₁₋₆ alkyl. In certain embodiments, R^{B} is optionally substituted with one or more R^{u}.

In certain embodiments, Ring C is C₆₋₁₀ aryl or 5- to 10-membered heteroaryl.

In certain embodiments, Ring C is C₆₋₁₀ aryl (e.g., phenyl or naphthyl).

In certain embodiments, Ring C is 5- to 10-membered heteroaryl comprising one or two 5- or 6-membered rings and 1-5 heteroatoms selected from N, O, and S. In certain embodiments, Ring C is 5- to 10-membered heteroaryl comprising one 5- or 6-membered ring and 1-3 heteroatoms selected from N, O, and S. In certain embodiments, Ring C is 5- to 10-membered heteroaryl comprising one 5-membered ring and 1-3 heteroatoms selected from N, O, and S. In certain embodiments, Ring C is 5- to 10-membered heteroaryl comprising one 6-membered ring and 1-3 heteroatoms selected from N, O, and S. In certain embodiments, Ring C is 5- to 10-membered heteroaryl comprising two 5- or 6-membered rings and 1-5 heteroatoms selected from N, O, and S. In certain embodiments, Ring C is 5- to 10-membered heteroaryl comprising two 5-membered rings and 1-5 heteroatoms selected from N, O, and S. In certain embodiments, Ring C is 5- to 10-membered heteroaryl comprising two 6-membered rings and 1-5 heteroatoms selected from N, O, and S. In certain embodiments, Ring C is 5- to 10-membered heteroaryl comprising one 5-membered ring and one 6-membered ring and 1-5 heteroatoms selected from N, O, and S.

In certain embodiments, Ring C is phenyl or pyridinyl.

In certain embodiments, R^{C1} is halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, R^{C1} is optionally substituted with one or more R^{u}.

In certain embodiments, R^{C1} is halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, R^{C1} is optionally substituted with one or more R^{u}.

In certain embodiments, R^{C1} is halogen or —OH. In certain embodiments, R^{C1} is halogen (e.g., —F, —Cl, —Br, or —I). In certain embodiments, R^{C1} is —Cl. In certain embodiments, R^{C1} is —OH.

In certain embodiments, R^{C1} is optionally substituted C₁₋₆ alkyl (e.g., methyl, ethyl, propyl, i-propyl, n-butyl, i-butyl, s-butyl, t-butyl, pentyl, or hexyl). In certain embodiments, R^{C1} is optionally substituted with one or more R^{u}.

In certain embodiments, each R^{C2} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{C2} is independently optionally substituted with one or more R^{u}.

In certain embodiments, each R^{C2} is independently halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{C2} is independently optionally substituted with one or more R^{u}.

In certain embodiments, r is 0. In certain embodiments, r is 1. In certain embodiments, r is 2. In certain embodiments, r is 3. In certain embodiments, r is 4. In certain embodiments, r is 5. In certain embodiments, r is 6.

In certain embodiments, Ring D is C₆₋₁₀ aryl or 5- to 10-membered heteroaryl.

In certain embodiments, Ring D is C₆₋₁₀ aryl (e.g., phenyl or naphthyl).

In certain embodiments, Ring D is 5- to 10-membered heteroaryl comprising one or two 5- or 6-membered rings and 1-5 heteroatoms selected from N, O, and S. In certain embodiments, Ring D is 5- to 10-membered heteroaryl comprising one 5- or 6-membered ring and 1-3 heteroatoms selected from N, O, and S. In certain embodiments, Ring D is 5- to 10-membered heteroaryl comprising one 5-membered ring and 1-3 heteroatoms selected from N, O, and S. In certain embodiments, Ring D is 5- to 10-membered heteroaryl comprising one 6-membered ring and 1-3 heteroatoms selected from N, O, and S. In certain embodiments, Ring D is 5- to 10-membered heteroaryl comprising two 5- or 6-membered rings and 1-5 heteroatoms selected from N, O, and S. In certain embodiments, Ring D is 5- to 10-membered heteroaryl comprising two 5-membered rings and 1-5 heteroatoms selected from N, O, and S. In certain embodiments, Ring D is 5- to 10-membered heteroaryl comprising two 6-membered rings and 1-5 heteroatoms selected from N, O, and S. In certain embodiments, Ring D is 5- to 10-membered heteroaryl comprising one 5-membered ring and one 6-membered ring and 1-5 heteroatoms selected from N, O, and S.

In certain embodiments, Ring D is phenyl, pyridinyl, pyrrolopyridazinyl, or thienopyridinyl.

In certain embodiments, R² is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-i-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —C(═O)NR^{c}S(═O)₂R^{a}, —C(═O)NR^{c}R^{d}, —(CH₂)C(═O)OR^{b}, or C(═O)OR^{b}, wherein the alkyl, alkenyl, alkynyl, alkoxy, alkylamino, carbocyclyl, heterocyclyl, aryl, or heteroaryl is optionally substituted. In certain embodiments, R² is optionally substituted with one or more R^{u}.

In certain embodiments, R² is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —C(═O)NR^{c}S(═O)₂R^{a}, —C(═O)NR^{c}R^{d}, —(CH₂)C(═O)OR^{b}, or C(═O)OR^{b}, wherein the alkyl, alkenyl, alkynyl, alkoxy, alkylamino, carbocyclyl, heterocyclyl, aryl, or heteroaryl is optionally substituted. In certain embodiments, R² is optionally substituted with one or more R^{u}.

In certain embodiments, R² is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, —C(═O)NR^{c}S(═O)₂R^{a}, —C(═O)NR^{c}R^{d}, —(CH₂)C(═O)OR^{b}, or C(═O)OR^{b}, wherein the alkyl, alkenyl, alkynyl, alkoxy, or alkylamino is optionally substituted. In certain embodiments, R² is optionally substituted with one or more R^{u}.

In certain embodiments, R² is hydrogen, halogen, —CN, —NO₂, —OH, —NH₂, C₁₋₆ alkyl, C₁₋₆ alkoxy, C₁₋₆ alkylamino, —C(═O)NR^{c}S(═O)₂R^{a}, —C(═O)NR^{c}R^{d}, —(CH₂)C(═O)OR^{b}, or C(═O)OR, wherein the alkyl, alkoxy, or alkylamino is optionally substituted. In certain embodiments, R² is optionally substituted with one or more R^{u}.

In certain embodiments, R² is hydrogen, halogen, C₁₋₆ alkyl, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, carbocyclyl, heterocyclyl, aryl, or heteroaryl is optionally substituted. In certain embodiments, R² is optionally substituted with one or more R^{u}.

In certain embodiments, R² is hydrogen, —C(═O)NR^{c}S(═O)R^{a}, —C(═O)NR^{c}R^{d}, —(CH₂)C(═O)OR^{b}, or —C(═O)OR^{b}. In certain embodiments, R² is —C(═O)NHS(═O)₂CH₃ or —COOH.

In certain embodiments, R² is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-i-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —C(═O)NR^{c}S(═O)₂R^{a}, or C(═O)OR^{b}, wherein the alkyl, alkenyl, alkynyl, alkoxy, alkylamino, carbocyclyl, heterocyclyl, aryl, or heteroaryl is optionally substituted. In certain embodiments, R² is optionally substituted with one or more R^{u}.

In certain embodiments, R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —C(═O)NR^{c}S(═O)₂R^{a}, or C(═O)OR^{b}, wherein the alkyl, alkenyl, alkynyl, alkoxy, alkylamino, carbocyclyl, heterocyclyl, aryl, or heteroaryl is optionally substituted. In certain embodiments, R² is optionally substituted with one or more R^{u}.

In certain embodiments, R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, —C(═O)NR^{c}S(═O)₂R^{a}, or C(═O)OR^{b}, wherein the alkyl, alkenyl, alkynyl, alkoxy, or alkylamino is optionally substituted. In certain embodiments, R² is optionally substituted with one or more R^{u}.

In certain embodiments, R² is halogen, —CN, —NO₂, —OH, —NH₂, C₁₋₆ alkyl, C₁₋₆ alkoxy, C₁₋₆ alkylamino, —C(═O)NR^{c}S(═O)₂R^{a}, or C(═O)OR^{b}, wherein the alkyl, alkoxy, or alkylamino is optionally substituted. In certain embodiments, R² is optionally substituted with one or more R^{u}.

In certain embodiments, R² is halogen, C₁₋₆ alkyl, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, carbocyclyl, heterocyclyl, aryl, or heteroaryl is optionally substituted. In certain embodiments, R² is optionally substituted with one or more R^{u}.

In certain embodiments, R² is —C(═O)NR^{c}S(═O)R^{a} or —C(═O)OR^{b}. In certain embodiments, R² is —C(═O)NHS(═O)₂CH₃ or —COOH.

In certain embodiments, each R^{D} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{D} is independently optionally substituted with one or more R^{u}.

In certain embodiments, each R^{D} is independently halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{D} is independently optionally substituted with one or more R^{u}.

In certain embodiments, each R^{D} is independently halogen or optionally substituted C₁₋₆ alkyl. In certain embodiments, at least one R^{D} is halogen. In certain embodiments, each R^{D} is independently halogen. In certain embodiments, at least one R^{D} is optionally substituted C₁₋₆ alkyl. In certain embodiments, each R^{D} is independently optionally substituted C₁₋₆ alkyl. In certain embodiments, each R^{D} is independently optionally substituted with one or more R^{u}.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6.

In certain embodiments, each -L- is independently —O—, —NR^{L}—, —CR^{L1}R^{L2}—, —CR^{L1}═CR^{L2}—, or —C≡C—. In certain embodiments, each -L- is independently —O—, —CR^{L1}R^{L2}—, or —C≡C—.

In certain embodiments, [L]_{q} is

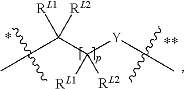

wherein:
* denotes attachment to Ring B, and ** denotes attachment to Ring C;
p is an integer selected from 0 to 3; and
Y is —O—, —CR^{L1}R^{L2}—, or —C≡C—.

In certain embodiments, L is Y.

In certain embodiments, each R^{L1} and each R^{L2} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), or C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), wherein the alkyl, alkoxy, or alkylamino is optionally substituted. In certain embodiments, each R^{L1} and each R^{L2} is independently optionally substituted with one or more R^{u}.

In certain embodiments, each R^{L1} and each R^{L2} is hydrogen.

In certain embodiments, p is 0. In certain embodiments, p is 1. In certain embodiments, p is 2. In certain embodiments, p is 3.

In certain embodiments, Y is —O—. In certain embodiments, Y is —NR^{L}—. In certain embodiments, Y is —CR^{L1}R^{L2}—. In certain embodiments, Y is —C≡C—. In certain embodiments, Y is —O— or —C≡C—.

In certain embodiments, Y is —O— and p is 0, or Y is —C≡C— and p is 0.

In certain embodiments, X is —O—. In certain embodiments, X is —C(R^{X})₂—.

In certain embodiments, each R^{X} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{X} is independently optionally substituted with one or more R^{u}.

In certain embodiments, each R^{X} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{X} is independently optionally substituted with one or more R^{u}.

In certain embodiments, each R^{X} is independently hydrogen or C₁₋₆ alkyl.

In certain embodiments, each R^{X} is independently C₁₋₆ alkyl.

In certain embodiments, each R^{X} is hydrogen.

In certain embodiments, each R^{X} is independently halogen or optionally substituted C₁₋₆ alkyl. In certain embodiments, at least one R^{X} is halogen. In certain embodiments, each R^{X} is independently halogen. In certain embodiments, at least one R^{X} is optionally substituted C₁₋₆ alkyl. In certain embodiments, each R^{X} is independently optionally substituted C₁₋₆ alkyl. In certain embodiments, each R^{X} is independently optionally substituted with one or more R^{u}.

In certain embodiments, two R^{X}, together with the carbon atom to which they are bonded, form an oxo.

In certain embodiments, two R^{X}, together with the carbon atom to which they are bonded, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted. In certain embodiments, two R^{X}, together with the carbon atom to which they are bonded, form C₃₋₆ carbocyclyl or 3- to 6-membered heterocyclyl, wherein the carbocyclyl or heterocyclyl is optionally substituted with one or more R^{u}.

In certain embodiments, two R^{X}, together with the carbon atom to which they are bonded, form C₃₋₄ carbocyclyl or 3- to 4-membered heterocyclyl.

In certain embodiments, each R^{A1} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{A1} is independently optionally substituted with one or more R^{u}.

In certain embodiments, each R^{A1} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{A1} is independently optionally substituted with one or more R^{u}.

In certain embodiments, each R^{A1} is independently hydrogen or C₁₋₆ alkyl.

In certain embodiments, each R^{A1} is independently C₁₋₆ alkyl.

In certain embodiments, each R^{A1} is hydrogen.

In certain embodiments, each R^{A1} is independently halogen or optionally substituted C₁₋₆ alkyl. In certain embodiments, at least one R^{A1} is halogen. In certain embodiments, each R^{A1} is independently halogen. In certain embodiments, at least one R^{A1} is optionally substituted C₁₋₆ alkyl. In certain embodiments, each R^{A1} is independently optionally substituted C₁₋₆ alkyl. In certain embodiments, each R^{A1} is independently optionally substituted with one or more R^{u}.

In certain embodiments, R^{X} and a vincinal R^{A1}, together with the carbon atoms to which they are bonded, form C₃₋₄ carbocyclyl or 3- to 4-membered heterocyclyl, wherein the carbocyclyl or heterocyclyl is optionally substituted. In certain embodiments, the carbocyclyl or heterocyclyl is optionally substituted with one or more R^{u}.

In certain embodiments, each occurrence of R^{a}, R^{b}, R^{c}, and R^{d} is independently and optionally substituted. In certain embodiments, each occurrence of R^{a}, R^{b}, R^{c}, and R^{d} is independently optionally substituted with one or more R^{u}.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl, or 5- to 10-membered heteroaryl, wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, or C₂₋₆ alkynyl, wherein the alkyl, alkenyl, or alkynyl is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl, or 5- to 10-membered heteroaryl, wherein the alkyl, alkenyl, alkynyl, carbocyclyl, heterocyclyl, aryl, or heteroaryl is optionally substituted with one or more R^{u}.

In certain embodiments, each R^{b} is independently C₁₋₆ alkyl, C₂₋₆ alkenyl, or C₂₋₆ alkynyl, wherein the alkyl, alkenyl, or alkynyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl, or 5- to 10-membered heteroaryl, wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₂₋₆ alkenyl, C₂₋₆ alkynyl, wherein the alkyl, alkenyl, or alkynyl is optionally substituted with one or more R^{u}.

In certain embodiments, R^{c} and R^{d}, together with the nitrogen atom to which they are bonded, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, each R^{u} is independently deuterium, oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy, ethoxy, propoxy, i-propoxy, n-butoxy, i-butoxy, s-butoxy, t-butoxy, pentoxy, or hexoxy), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NRS(═O)₂R^{a}, —NRS(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently deuterium, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently deuterium, oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently deuterium, oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the compound disclosed herein is selected from the compounds in Table 1 and pharmaceutically acceptable salts, solvates, stereoisomers, or prodrugs thereof.

TABLE 1
For all compounds, the assigned configurations at chiral centers are arbitrarily assigned

| Compound | |
|---|---|
| No. | Chemical Structure |
| 1 | 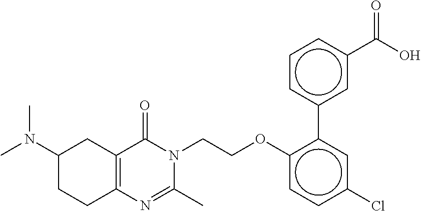 |
| 2 | 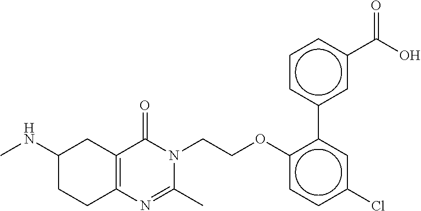 |
| 3 | 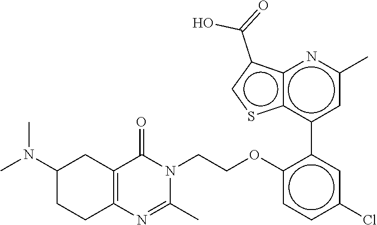 |
| 4 | 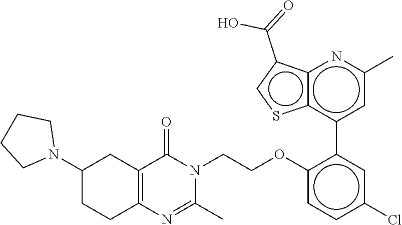 |
| 5* | 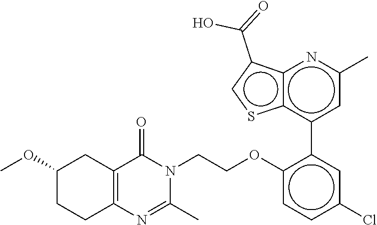 |
| 6* | 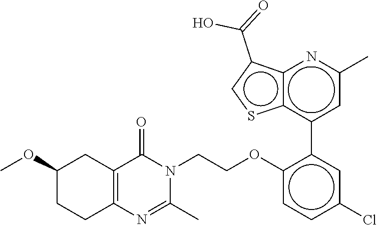 |
| 7 | 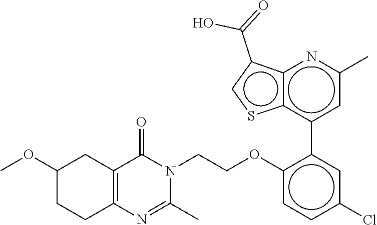 |
| 8 | 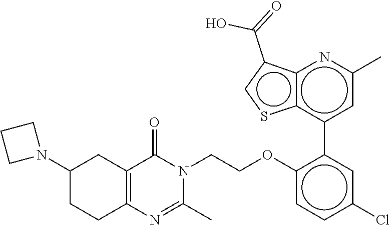 |
| 9 | 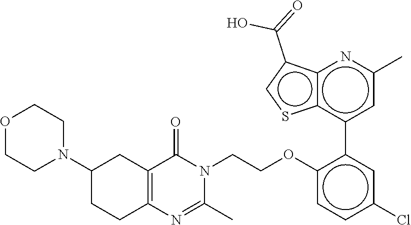 |
| 10 | 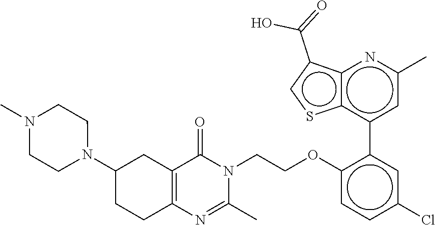 |
| 11 | 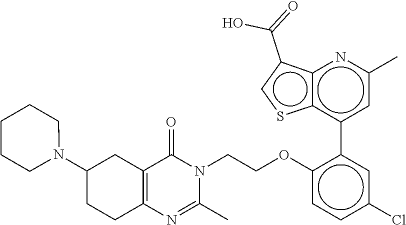 |
| 12 | 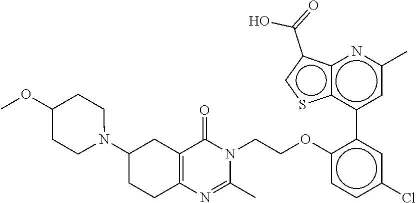 |
| 13* | 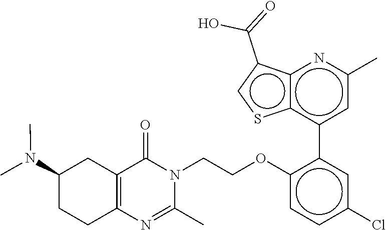 |
| 14* | 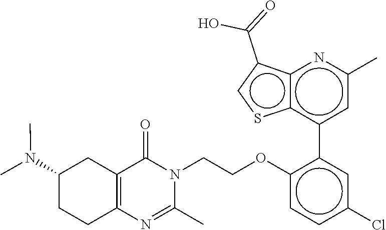 |
| 15 | 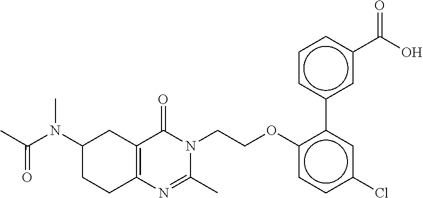 |
| 16 | 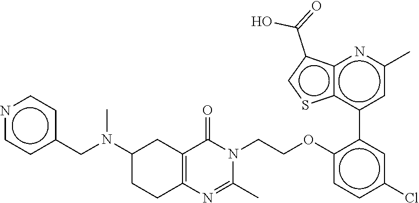 |
| 17 | 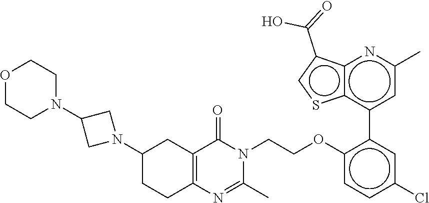 |
| 18 | 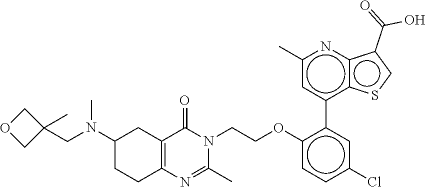 |
| 19 | 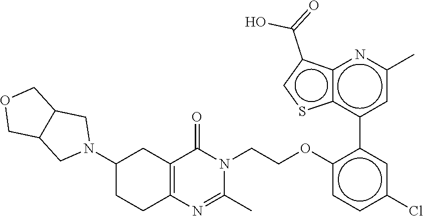 |
| 20 | 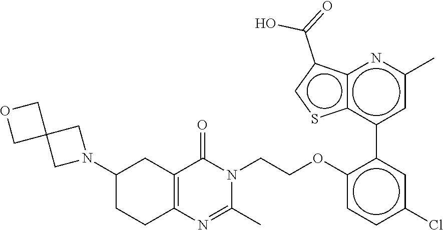 |
| 21 | 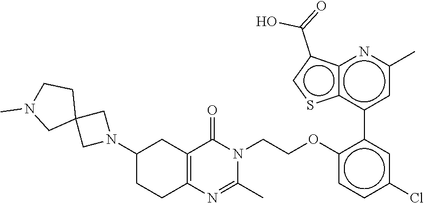 |
| 22 | 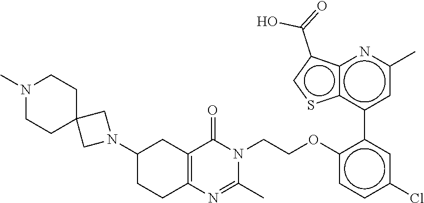 |
| 23 | 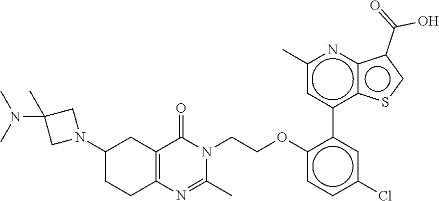 |
| 24* | 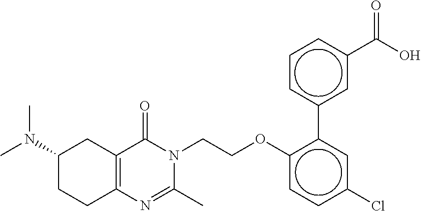 |
| 25* | 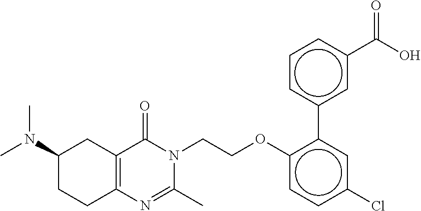 |
| 26 | 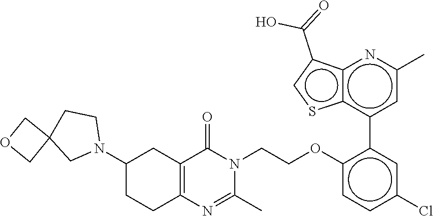 |
| 27 | 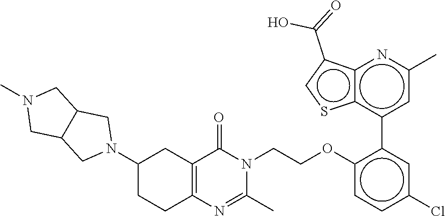 |
| 28 | 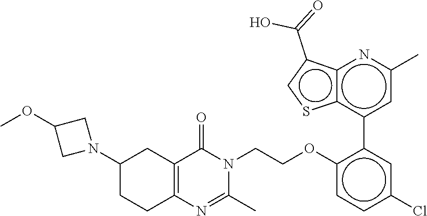 |
| 29 | 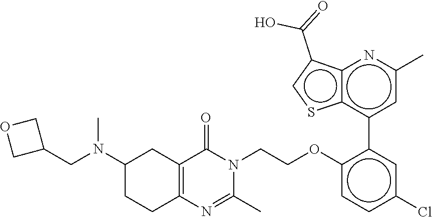 |
| 30 | 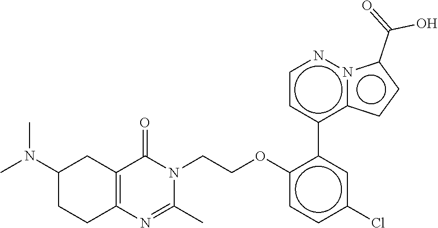 |
| 31 | 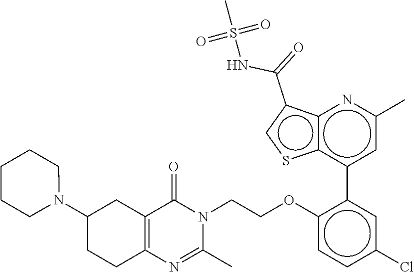 |
| 32 | 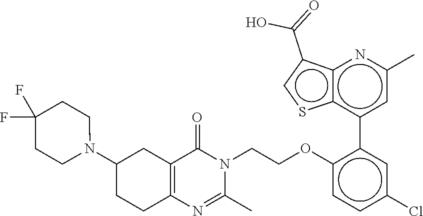 |
| 33 | 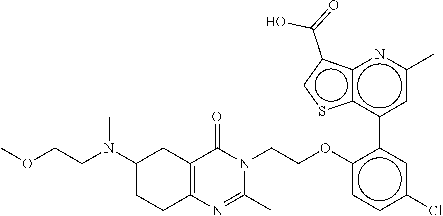 |
| 34 | 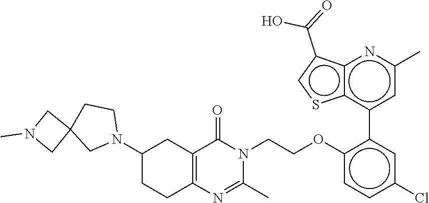 |
| 35 | 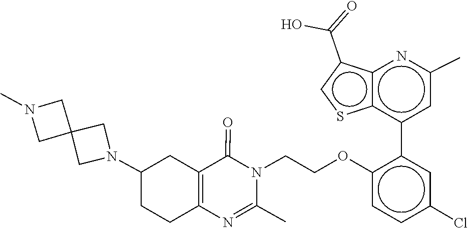 |
| 36 | 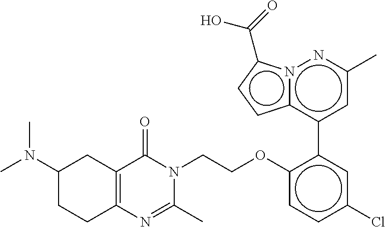 |
| 37 | 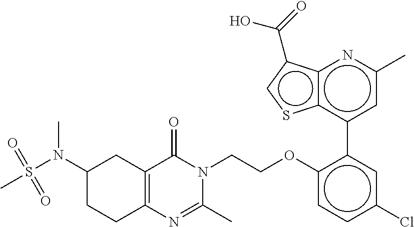 |
| 38 | 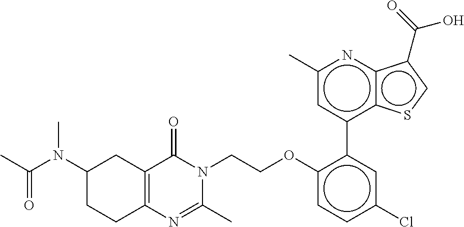 |
| 39 | 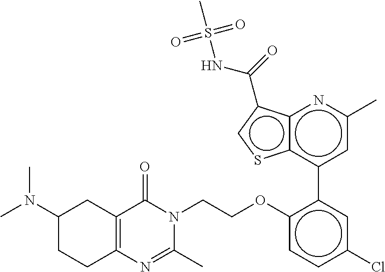 |
| 40 | 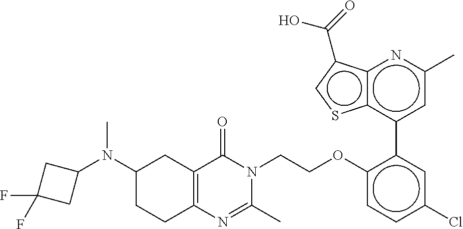 |
| 41 | 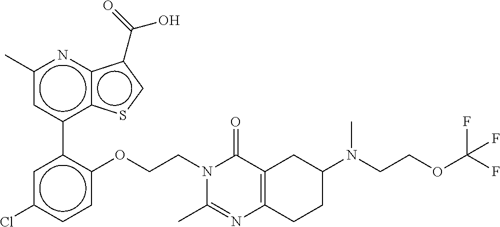 |
| 42* | 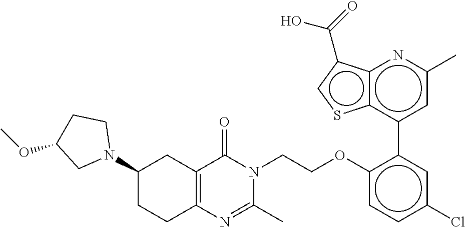 |
| 43* | 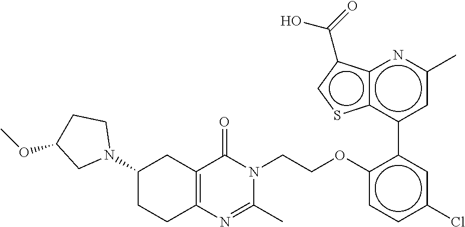 |
| 44* | 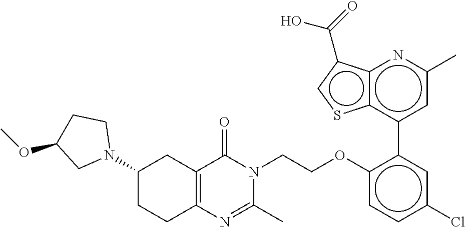 |
| 45 | 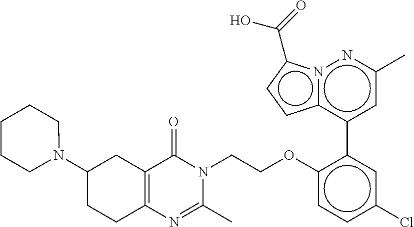 |
| 46 | 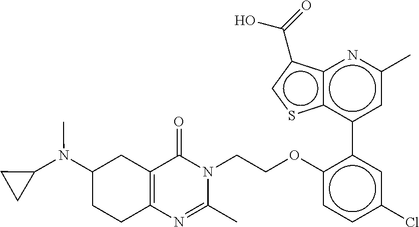 |
| 47 | 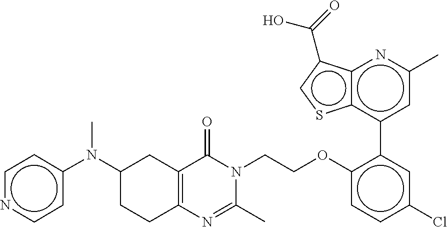 |
| 48* | 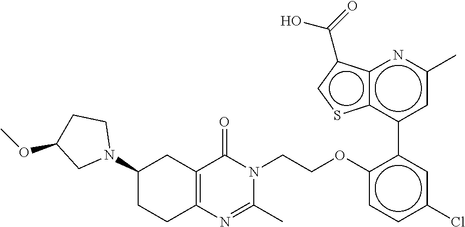 |
| 49 | 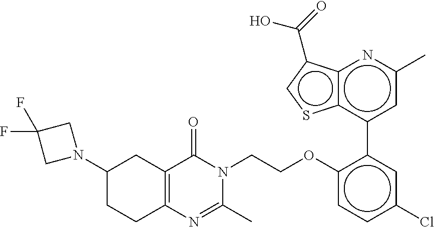 |
| 50 | 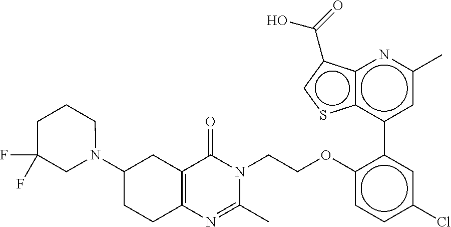 |
| 51 | 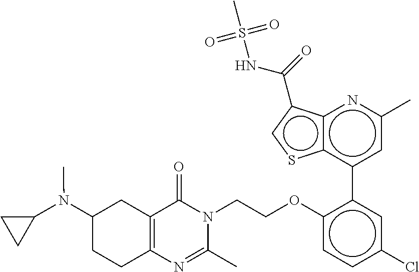 |
| 52 | 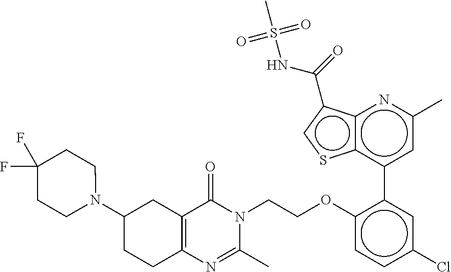 |
| 53 | 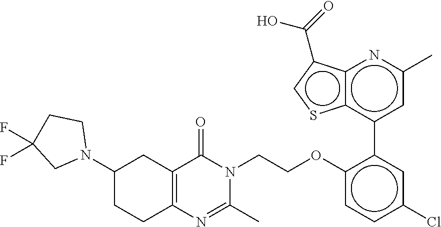 |
| 54 | 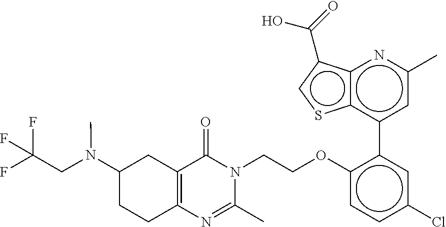 |
| 55 | 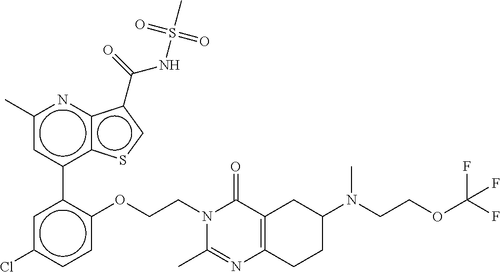 |
| 56 | 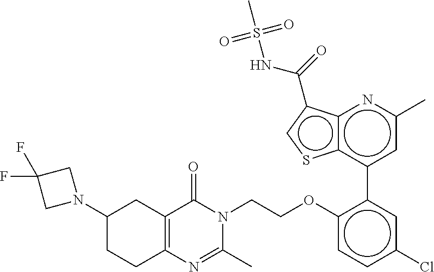 |
| 57 | 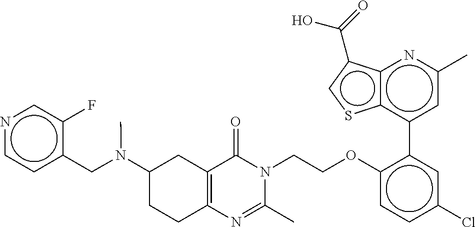 |
| 58 | 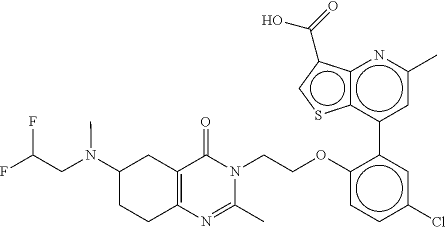 |
| 59 | 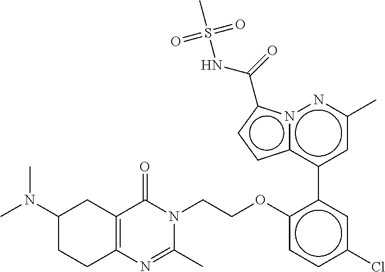 |
| 60 | 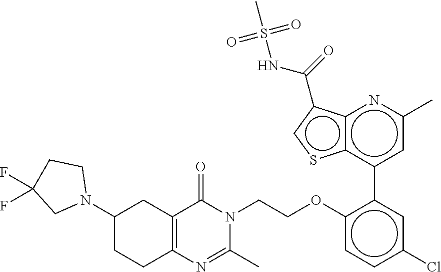 |
| 61 | 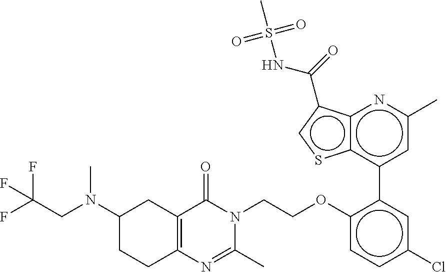 |
| 62 | 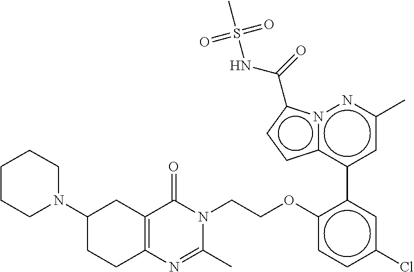 |
| 63 | 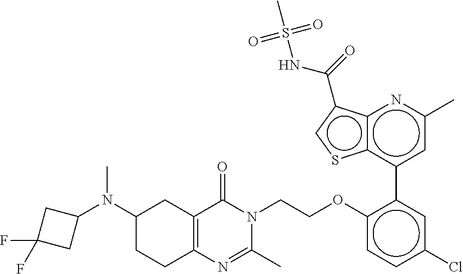 |
| 64 | 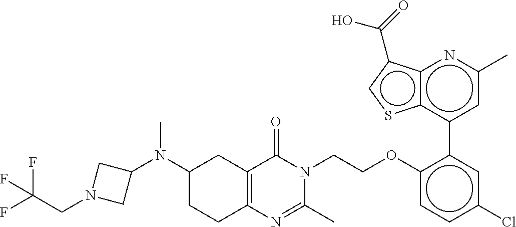 |
| 65 | 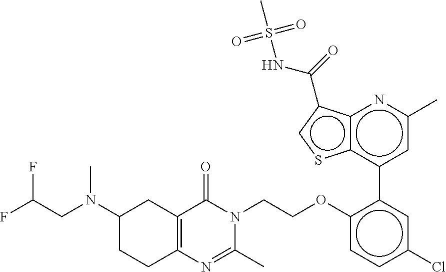 |
| 66 | 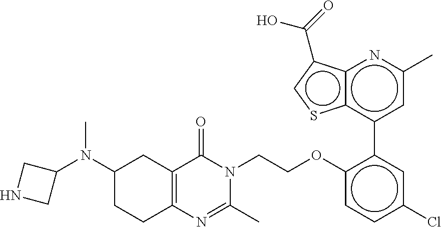 |
| 67* | 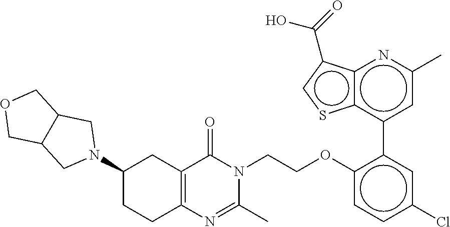 |
| 68 | 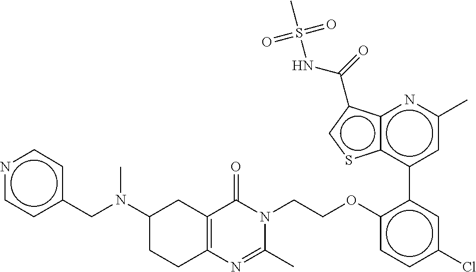 |
| 69 | 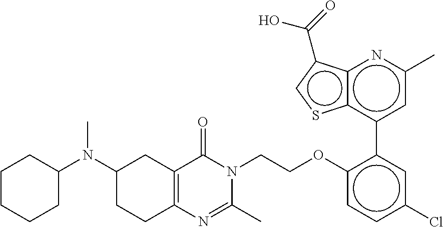 |
| 70* | 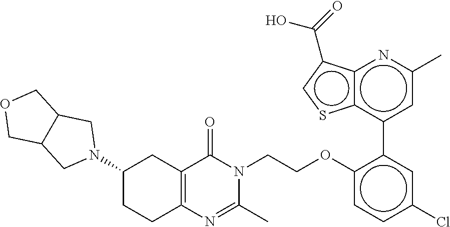 |
| 71 | 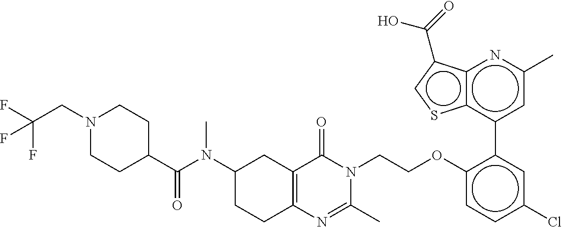 |
| 72 | 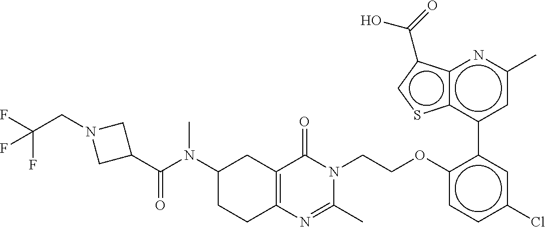 |
| 73 | 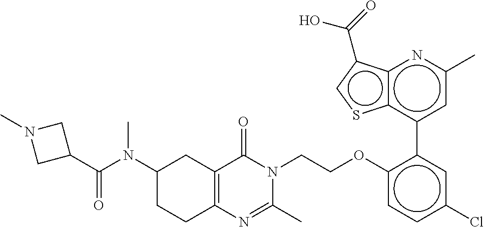 |
| 74 | 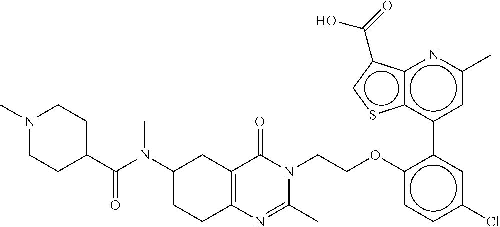 |
| 75 | 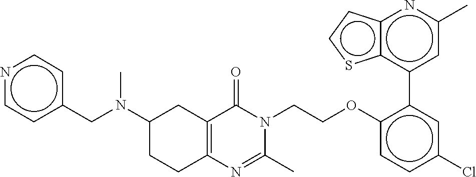 |
| 76 | 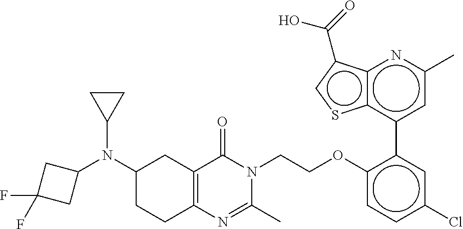 |
| 77 | 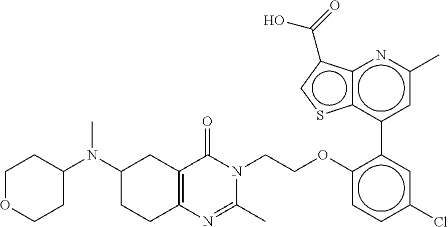 |
| 78 | 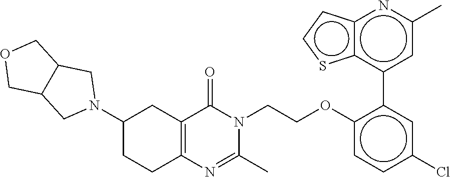 |
| 79 | 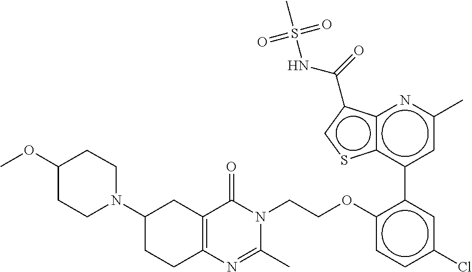 |
| 80 | 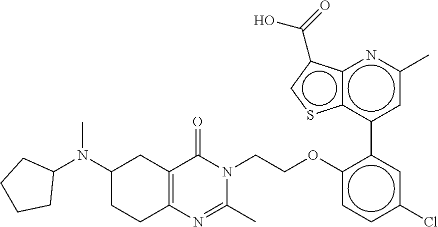 |
| 81 | 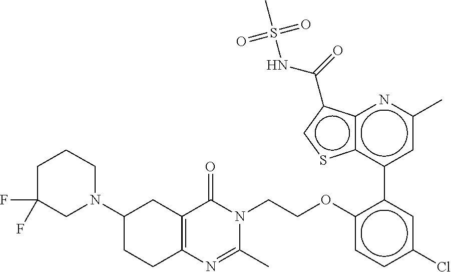 |
| 82 | 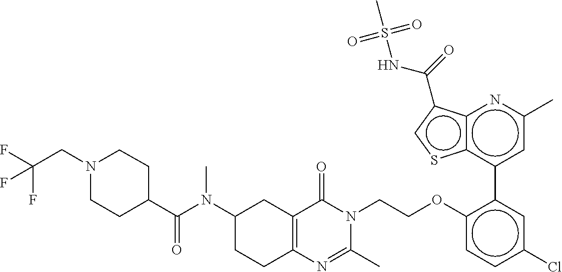 |
| 83 | 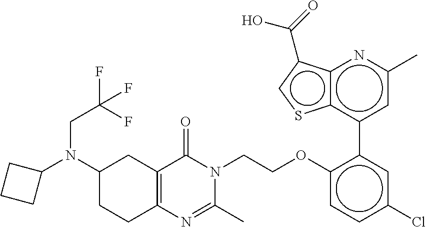 |
| 84 | 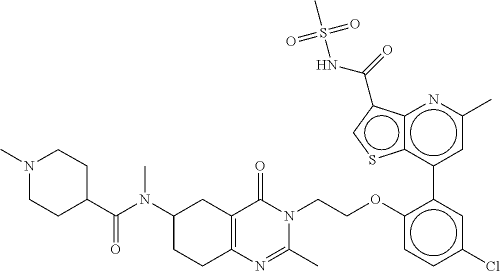 |
| 85 | 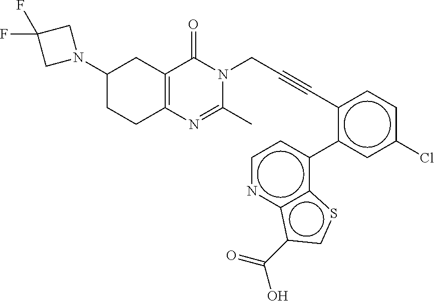 |
| 86 | 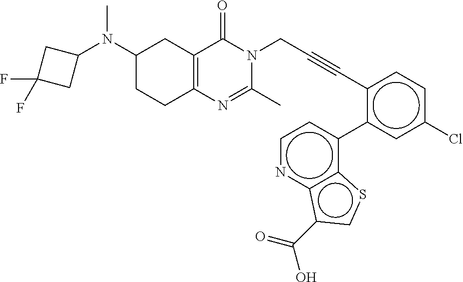 |
| 87 | 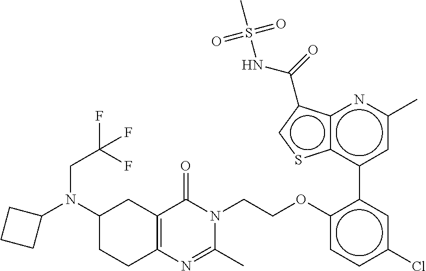 |
| 88 | 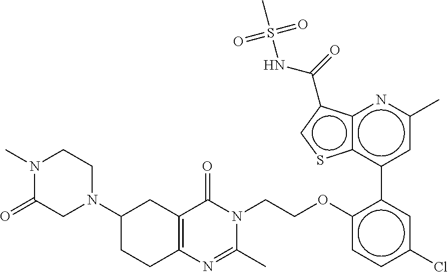 |
| 89* | 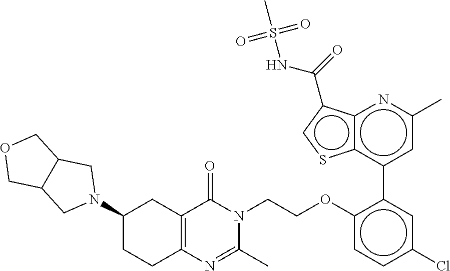 |
| 90 | 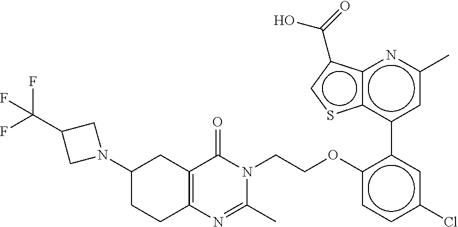 |
| 91 | 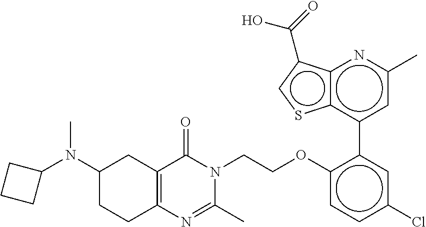 |
| 92 | 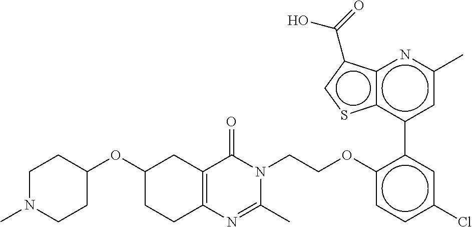 |
| 93* | 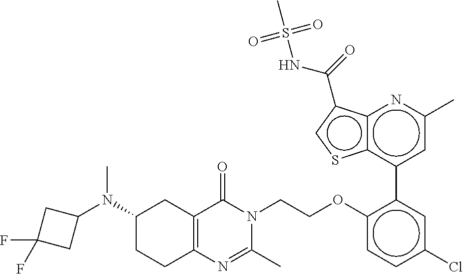 |
| 94* | 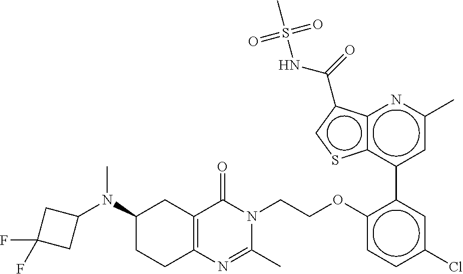 |
| 95 | 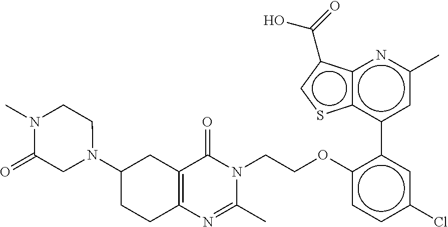 |
| 96 | 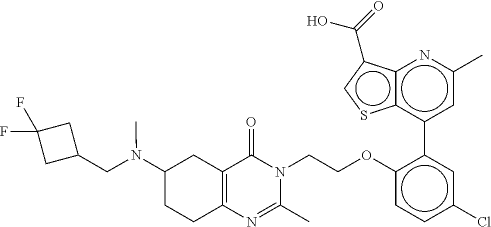 |
| 97 | 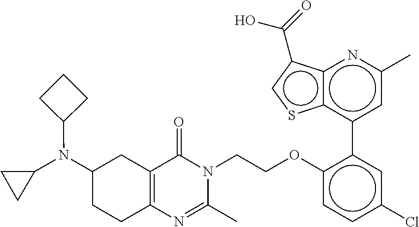 |
| 98 | 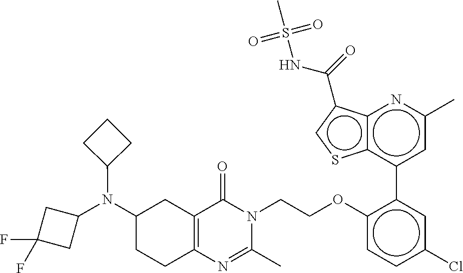 |
| 99* | 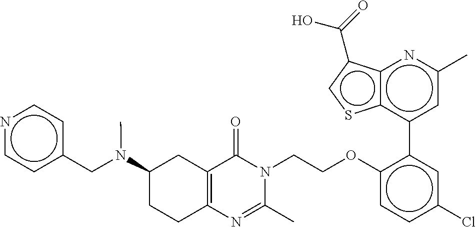 |
| 100* | 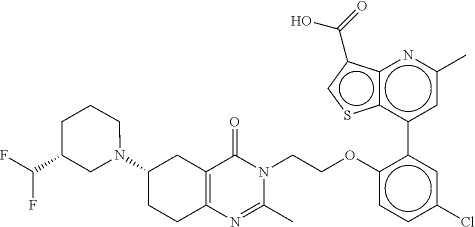 |
| 101 | 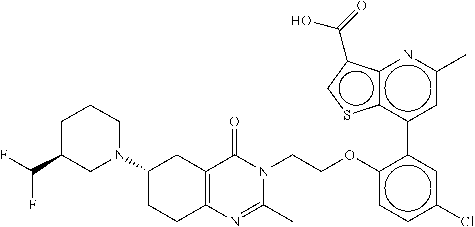 |
| 102 | 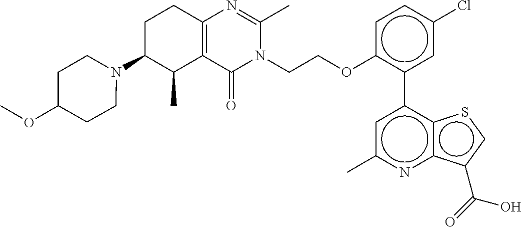 |
| 103 | 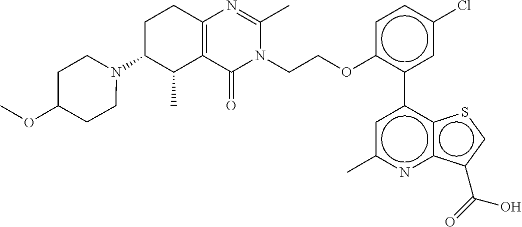 |
| 104 | 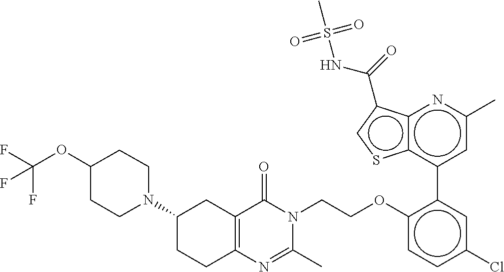 |
| 105 | 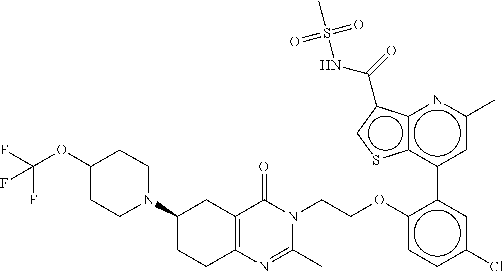 |
| 106 | 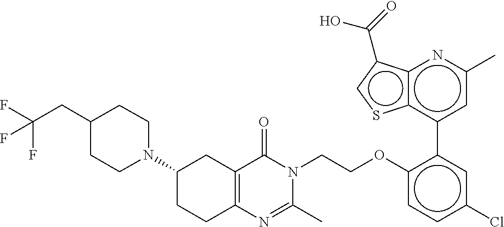 |
| 107 | 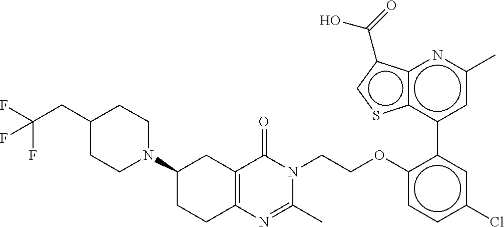 |
| 108 | 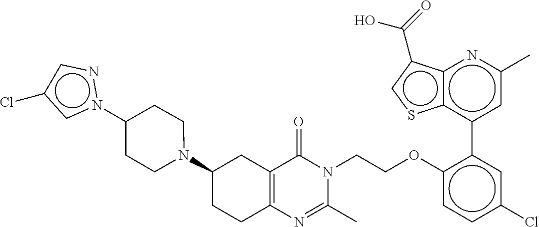 |
| 109 | 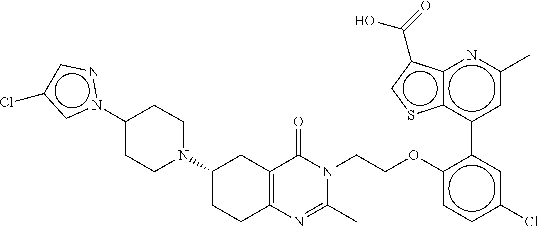 |
| 110 | 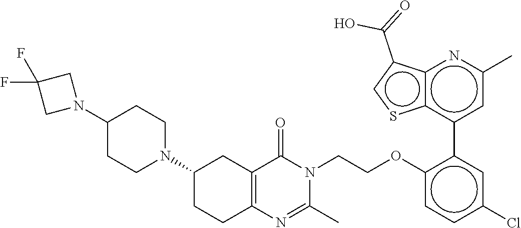 |
| 111 | 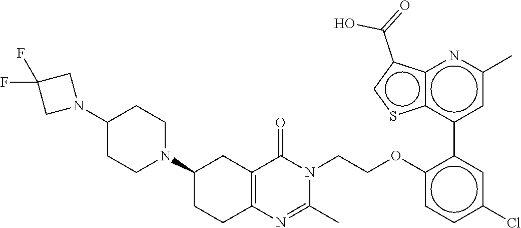 |
| 112 | 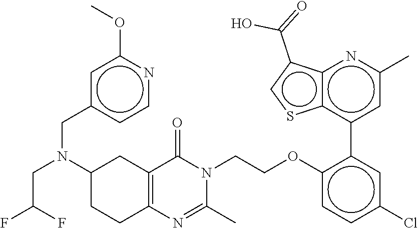 |
| 113 | 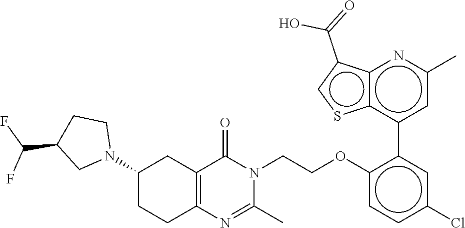 |
| 114 | 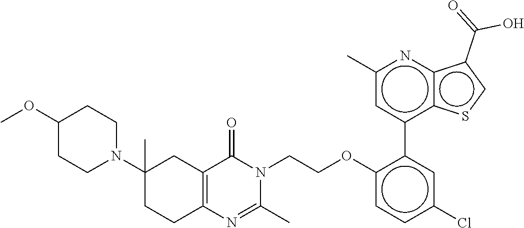 |
| 115 | 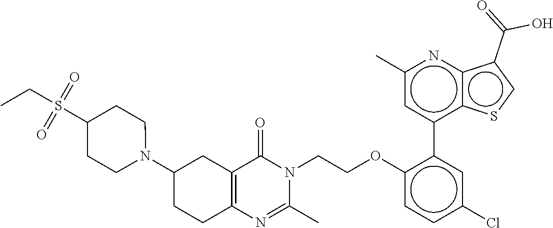 |
| 116 | 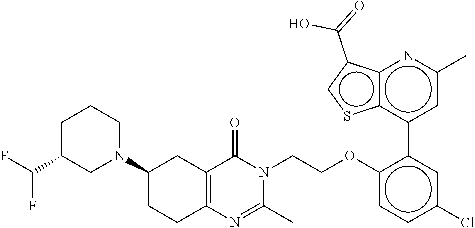 |
| 117 | 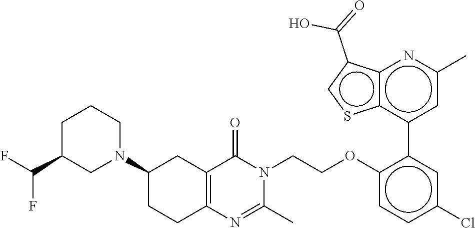 |
| 118 | 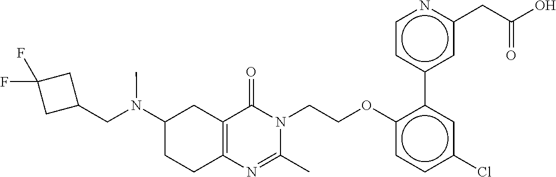 |
| 119 | 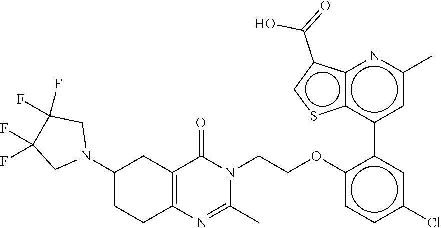 |
| 120 | 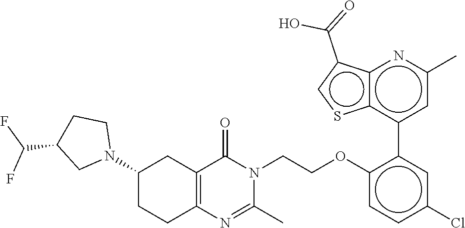 |
| 121 | 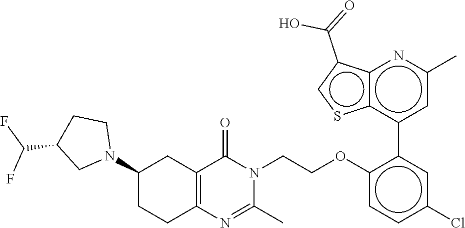 |
| 122 | 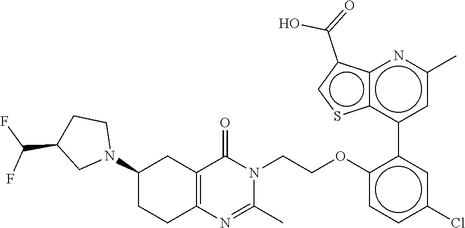 |
| 123 | 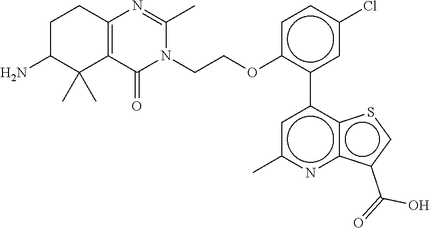 |
| 124 | 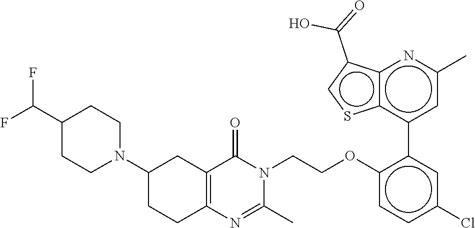 |
| 125 | 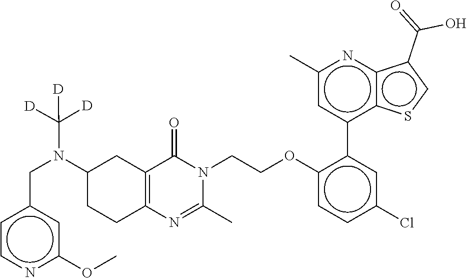 |
| 126 | 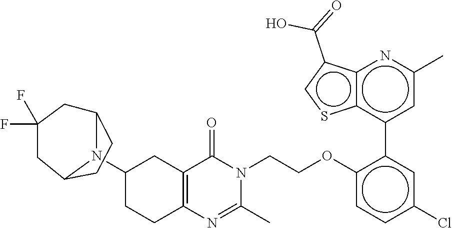 |
| 127 | 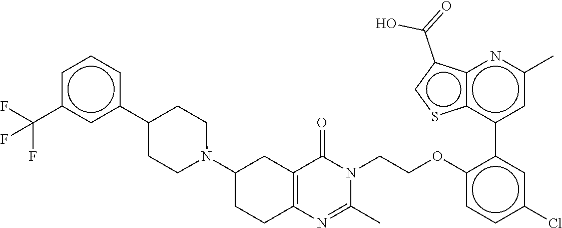 |
| 128 | 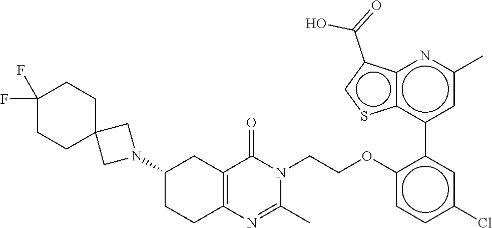 |
| 129 | 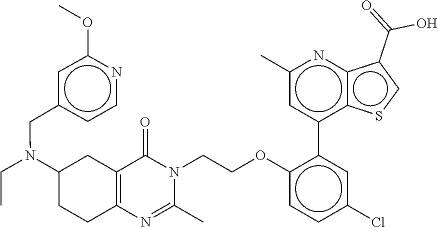 |
| 130 | 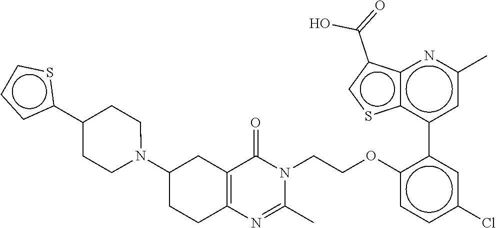 |
| 131 | 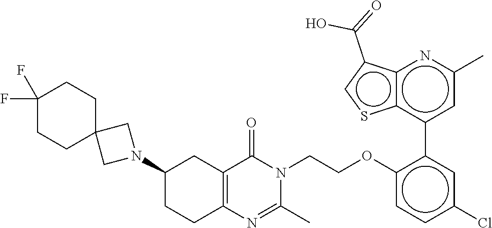 |
| 132 | 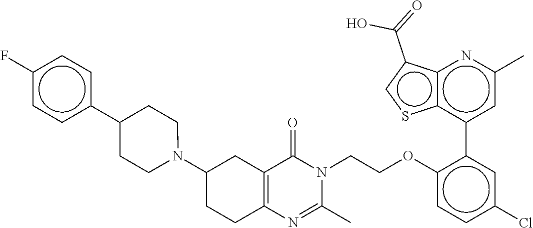 |
| 133 | 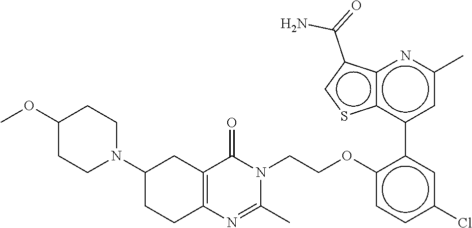 |
| 134 | 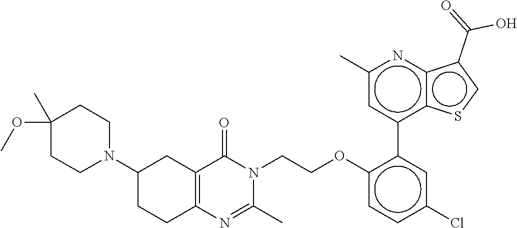 |
| 135 | 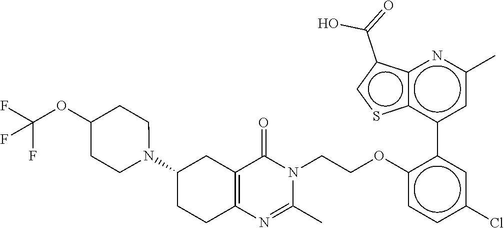 |
| 136 | 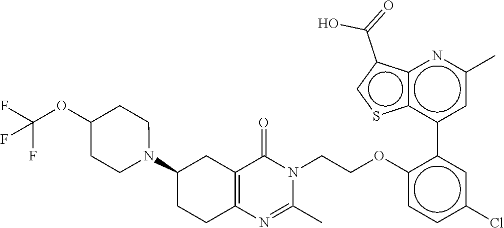 |
| 137 | 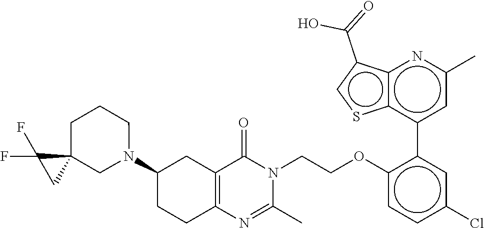 |
| 138 | 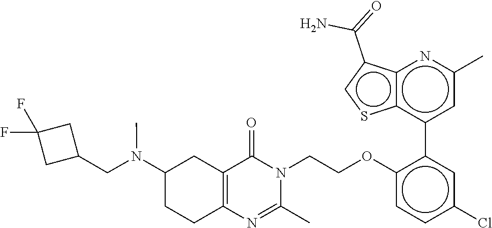 |
| 139 | 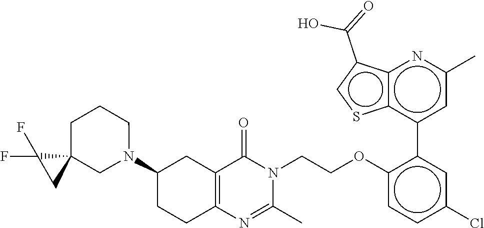 |
| 140 | 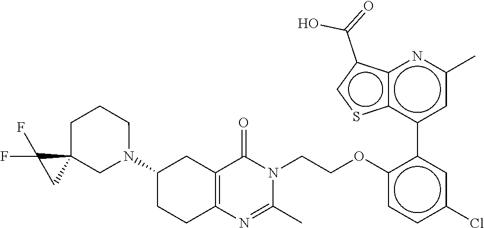 |
| 141 | 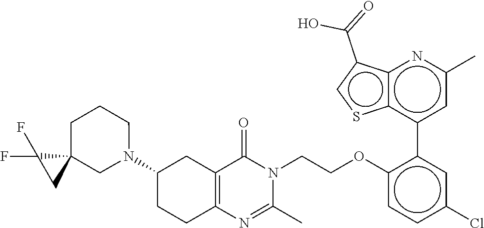 |
| 142 | 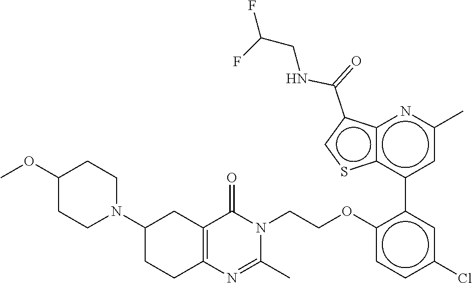 |
| 143 | 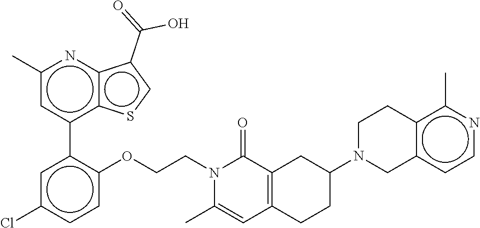 |
| 144 | 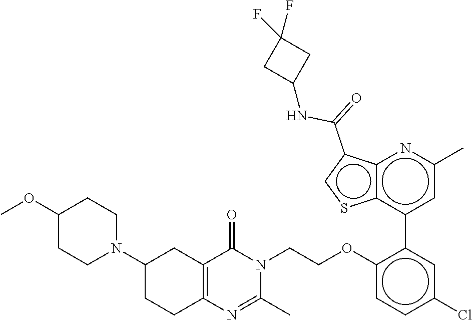 |
| 145 | 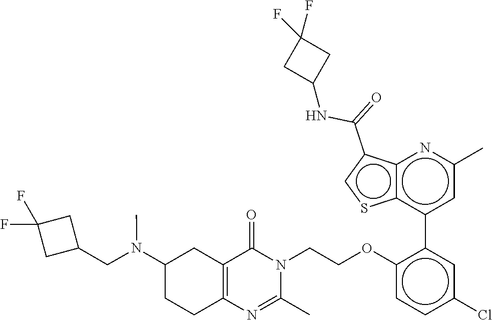 |
| 146 | 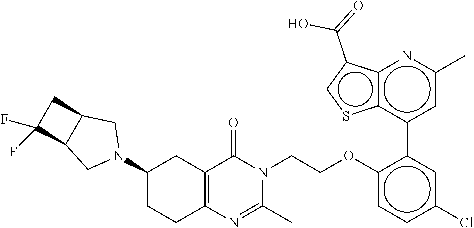 |
| 147 | 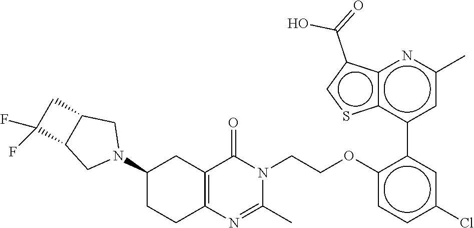 |
| 148 | 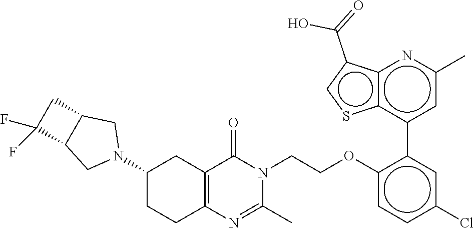 |
| 149 | 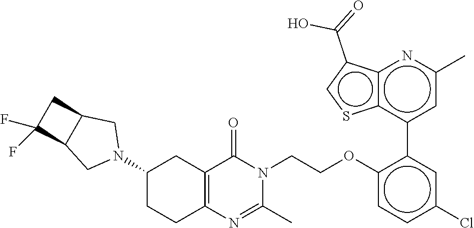 |
| 150 | 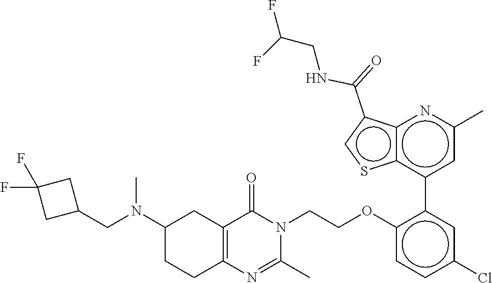 |
| 151 | 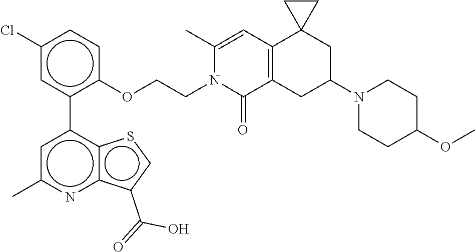 |
| 152 | 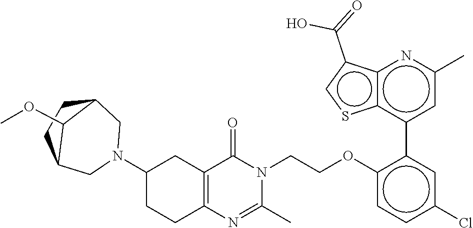 |
| 153 | 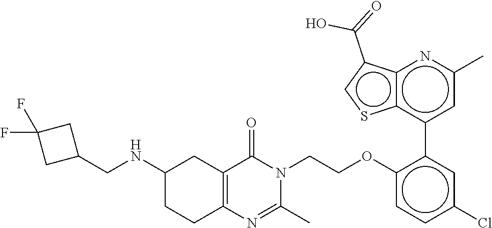 |
| 154 | 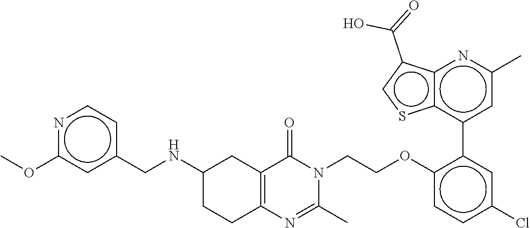 |
| 155 | 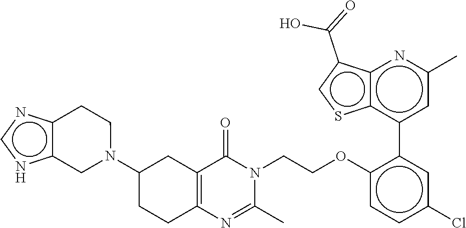 |
| 156 | 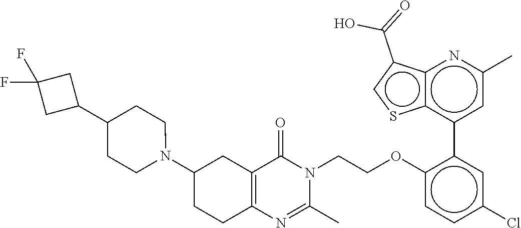 |
| 157 | 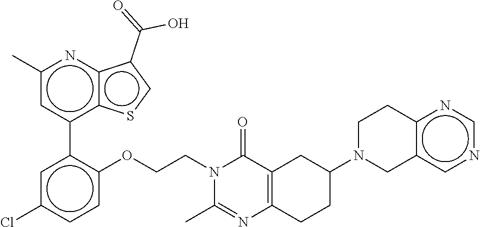 |
| 158 | 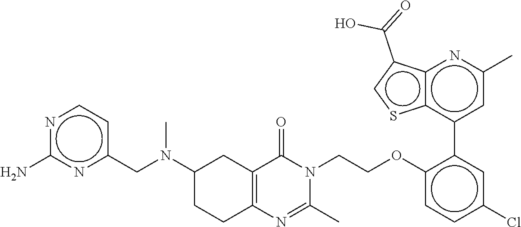 |
| 159 | 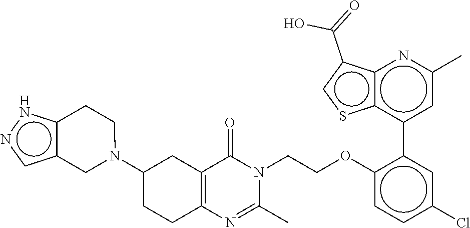 |
| 160 | 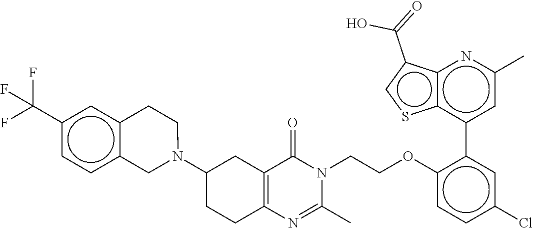 |
| 161 | 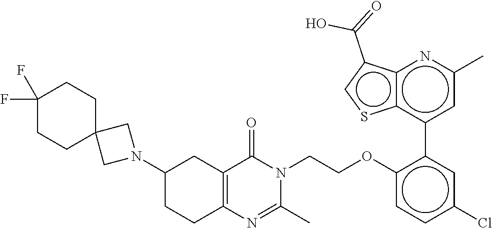 |
| 162 | 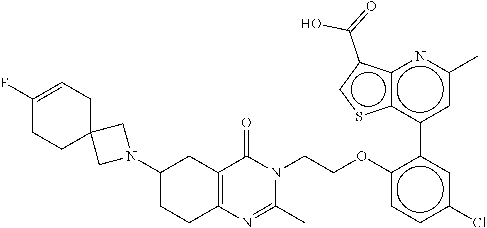 |
| 163 | 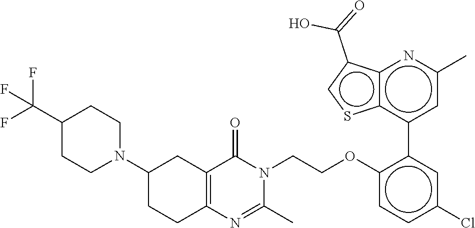 |
| 164 | 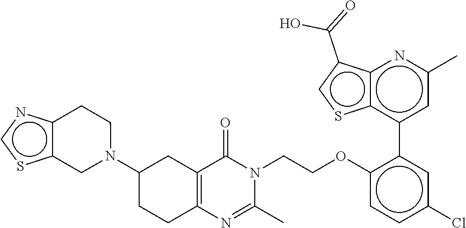 |
| 165 | 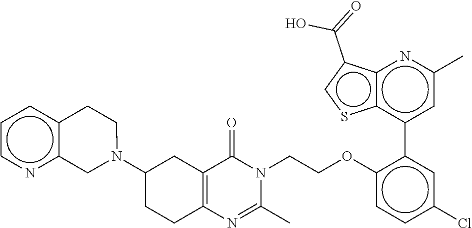 |
| 166 | 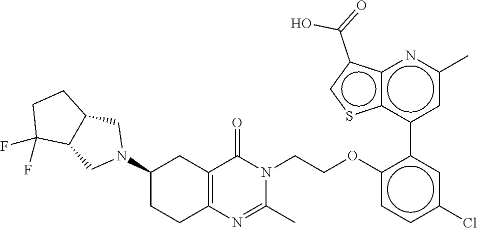 |
| 167 | 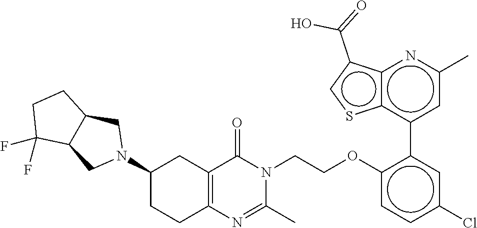 |
| 168 | 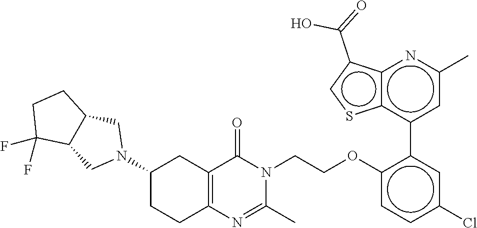 |
| 169 | 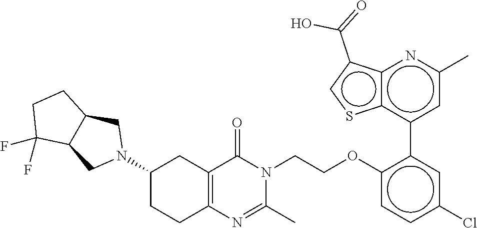 |
| 170 | 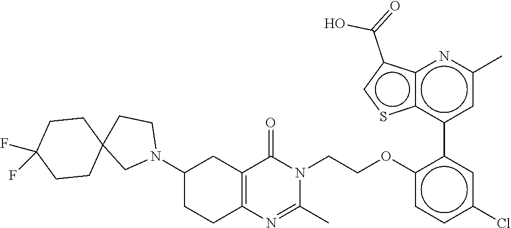 |
| 171 | 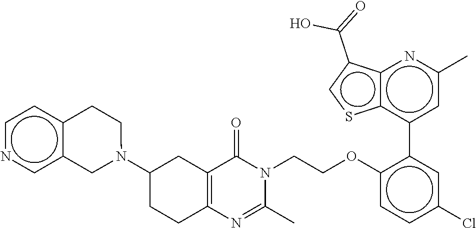 |
| 172 | 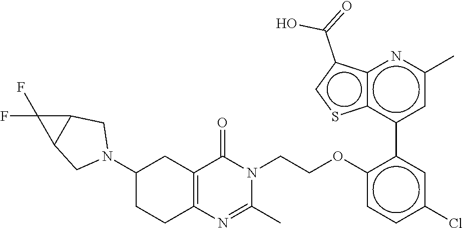 |
| 173 | 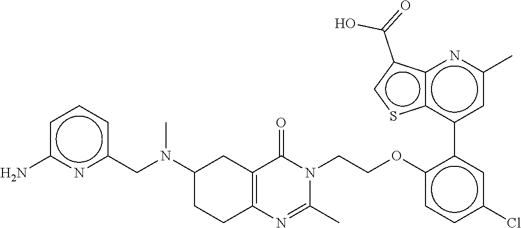 |
| 174 | 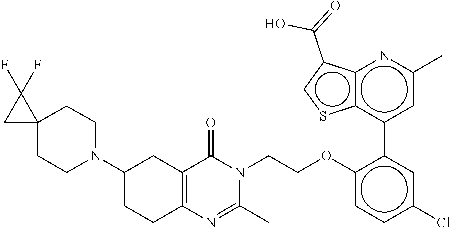 |
| 175 | 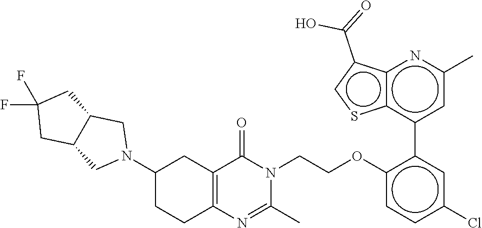 |
| 176 | 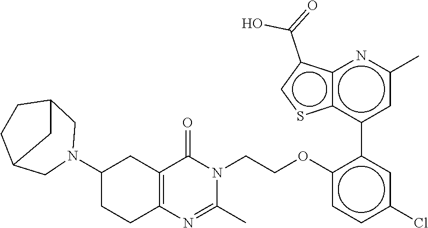 |
| 177 | 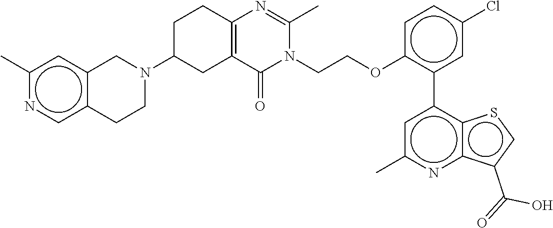 |
| 178 | 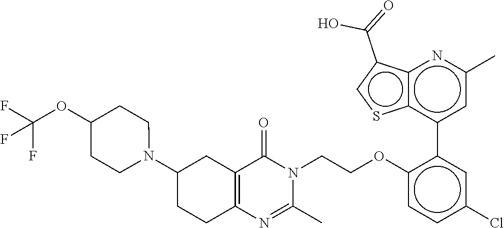 |
| 179 | 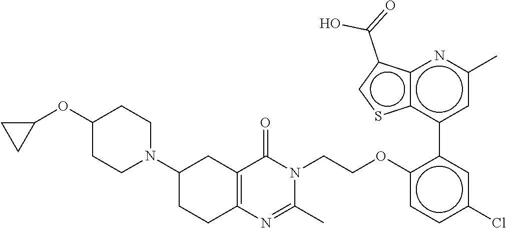 |
| 180 | 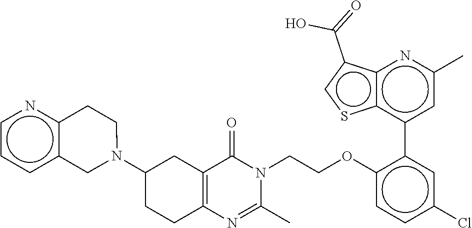 |
| 181 | 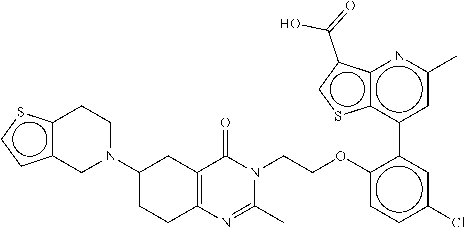 |
| 182 | 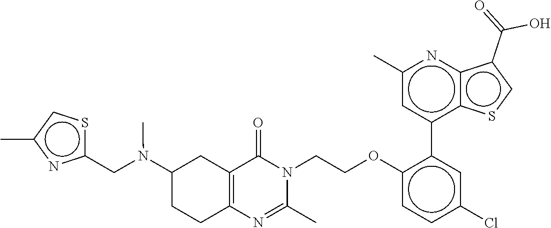 |
| 183 | 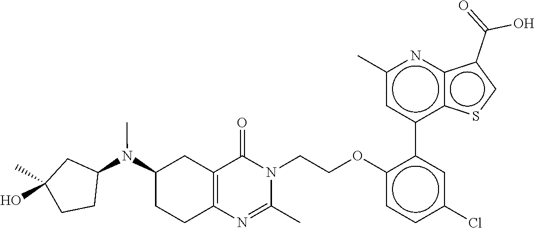 |
| 184 | 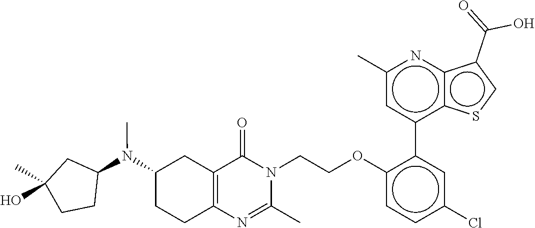 |
| 185 | 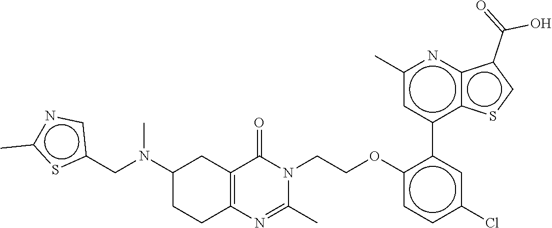 |
| 186 | 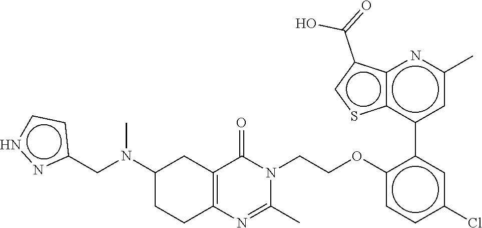 |
| 187 | 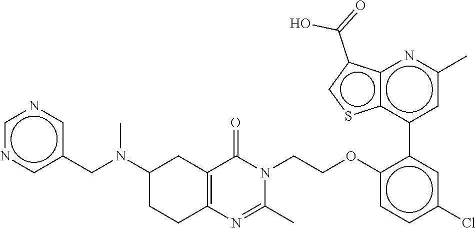 |
| 188 | 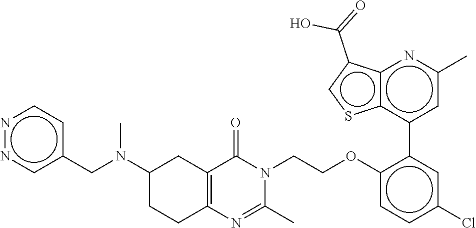 |
| 189 | 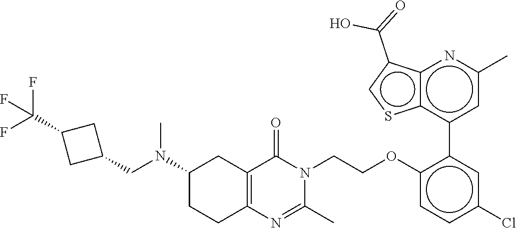 |
| 190 | 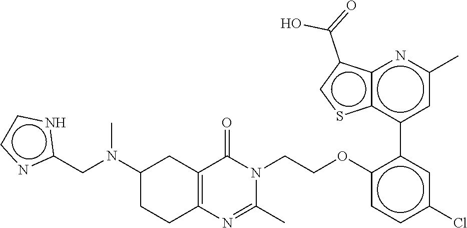 |
| 191 | 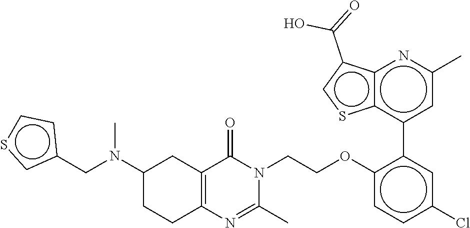 |
| 192 | 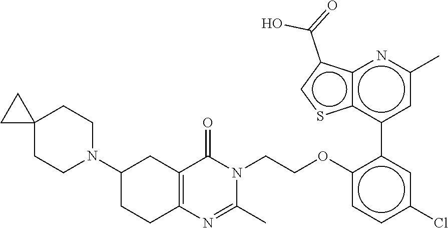 |
| 193 | 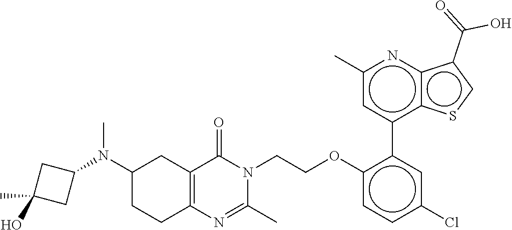 |
| 194 | 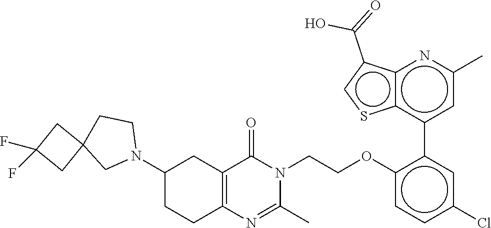 |
| 195 | 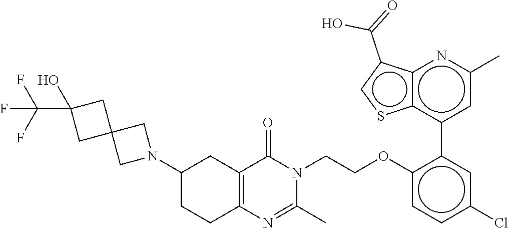 |
| 196 | 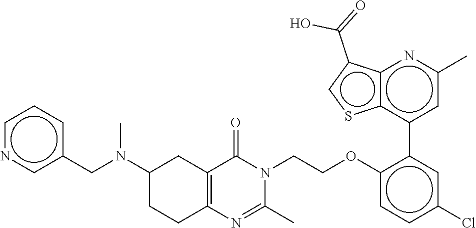 |
| 197 | 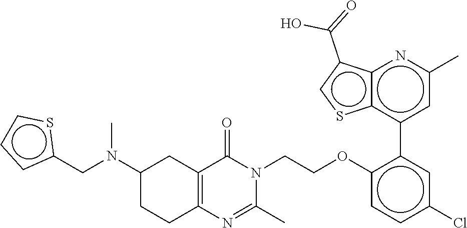 |
| 198 | 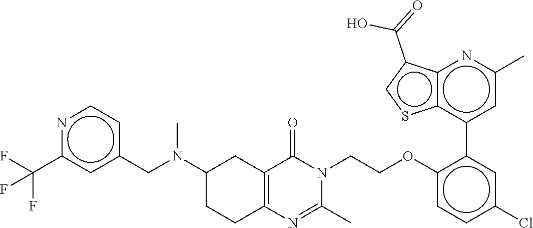 |
| 199 | 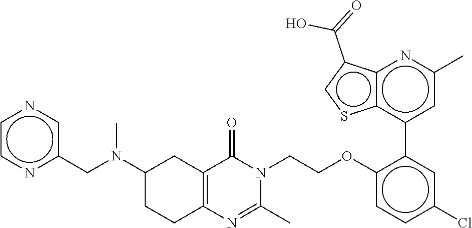 |
| 200 | 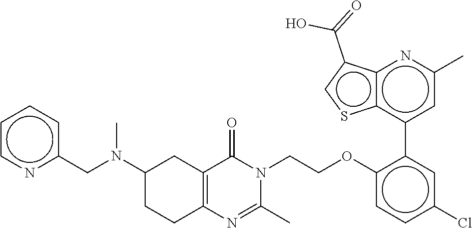 |
| 201 | 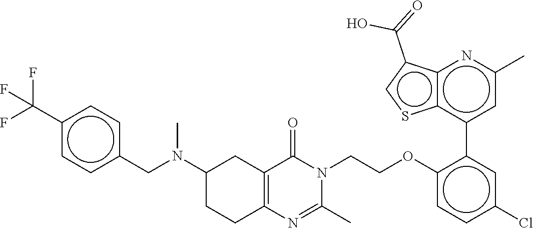 |
| 202 | 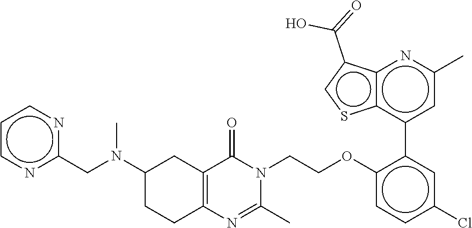 |
| 203 | 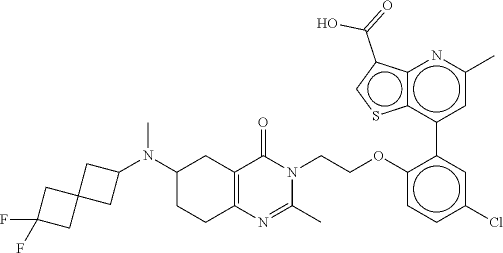 |
| 204 | 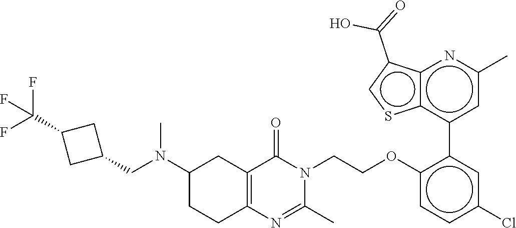 |
| 205 | 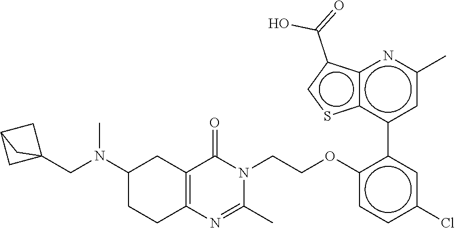 |
| 206 | 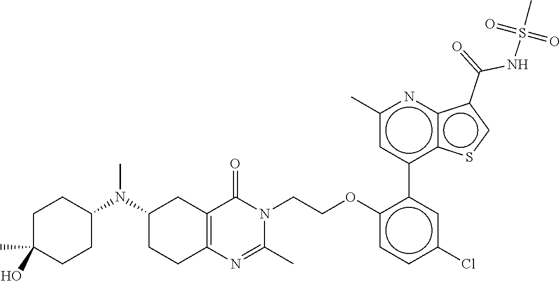 |
| 207 | 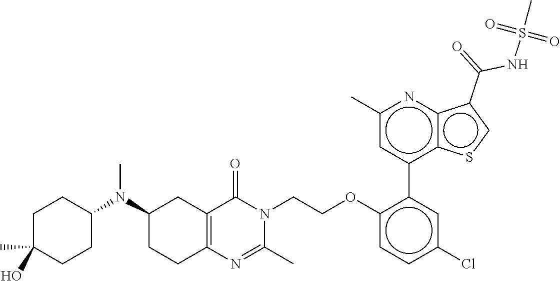 |
| 208 | 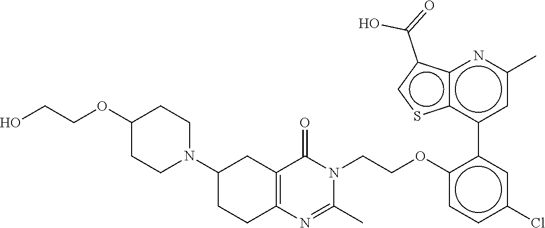 |
| 209 | 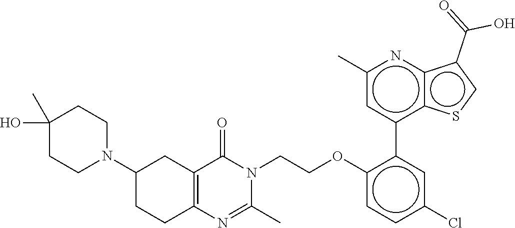 |
| 210 | 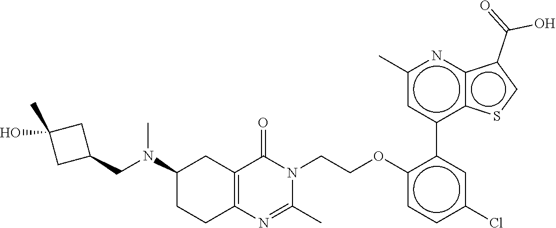 |
| 211 | 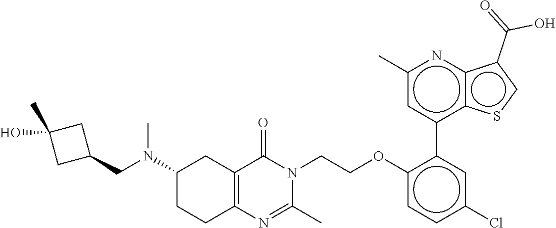 |
| 212 | 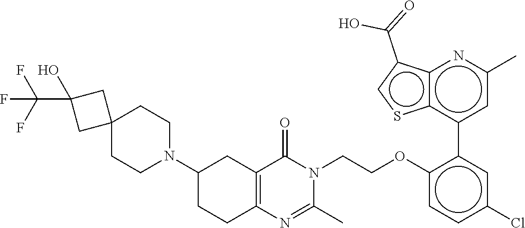 |
| 213 | 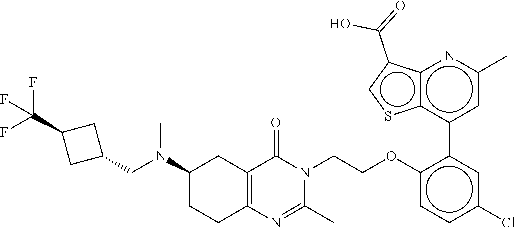 |
| 214 | 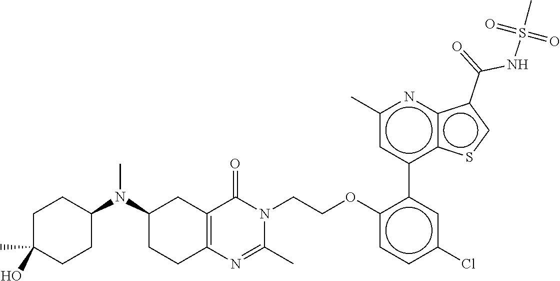 |
| 215 | 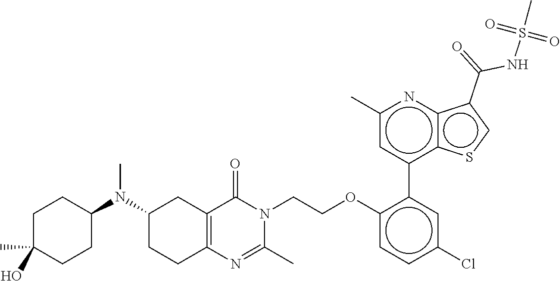 |
| 216 | 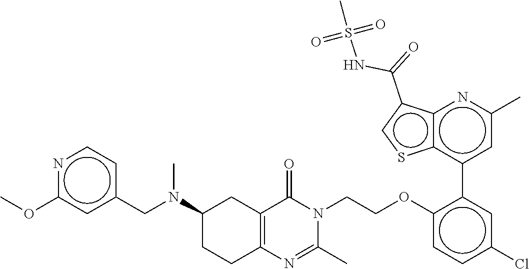 |
| 217 | 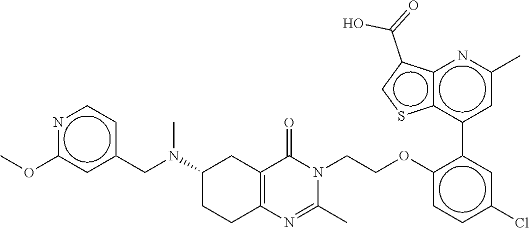 |
| 218 | 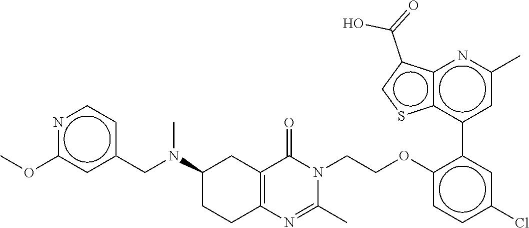 |
| 219 | 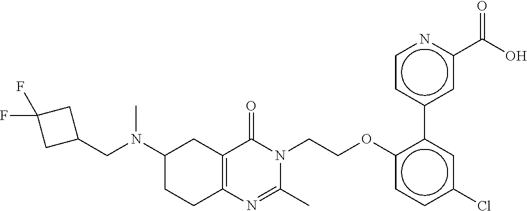 |
| 220 | 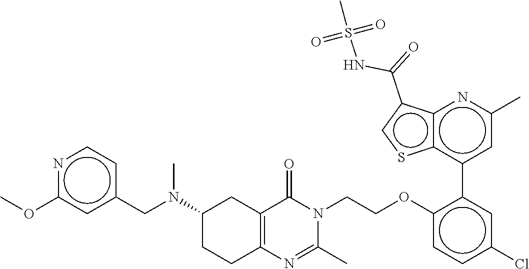 |
| 221 | 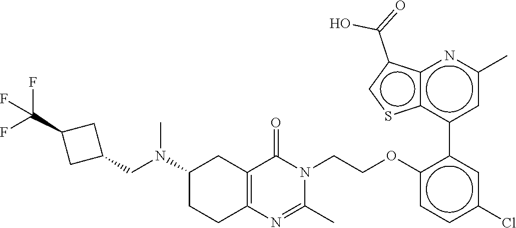 |
| 222 | 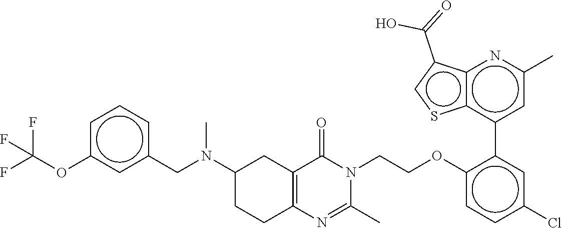 |
| 223 | 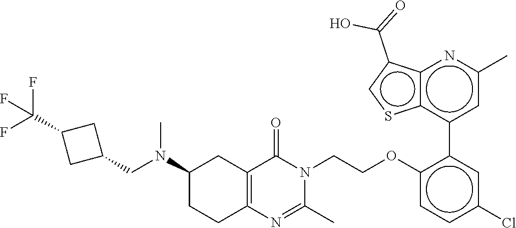 |
| 224 | 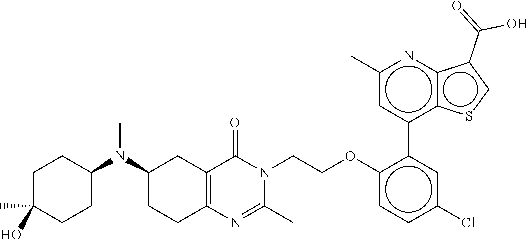 |
| 225 | 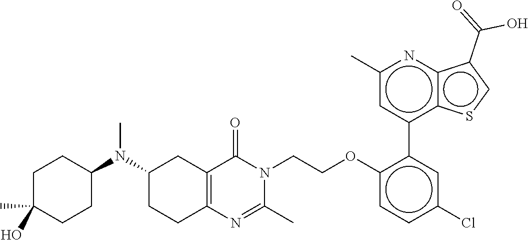 |
| 226 | 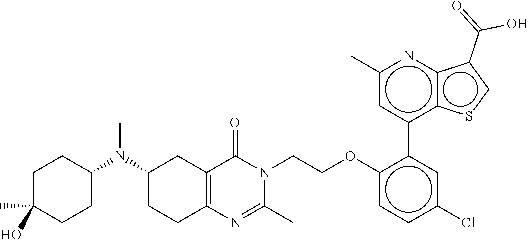 |
| 227 | 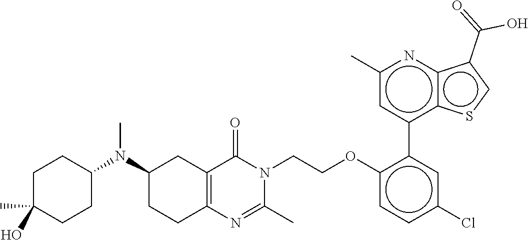 |
| 228 | 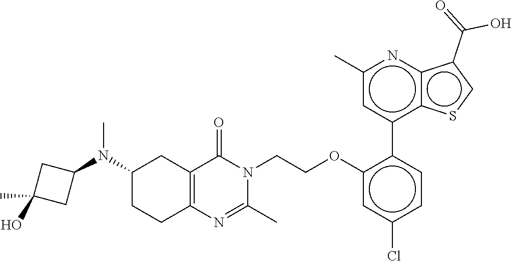 |
| 229 | 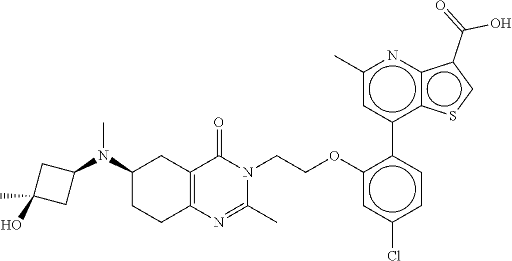 |
| 230 | 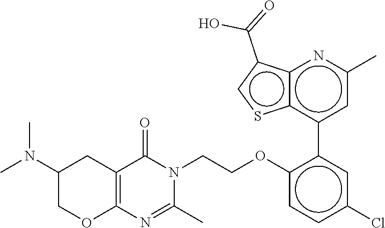 |
| 231 | 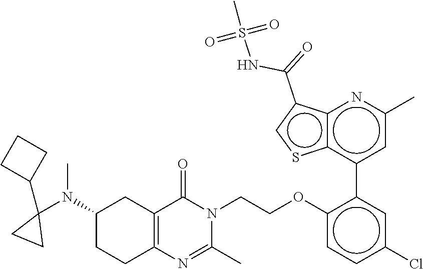 |
| 232 | 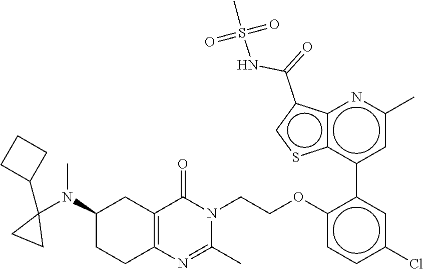 |
| 233 | 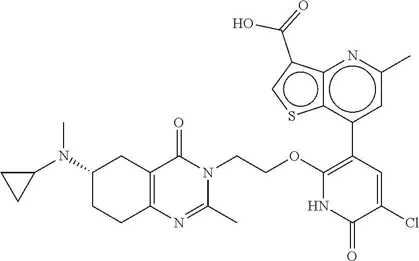 |
| 234 | 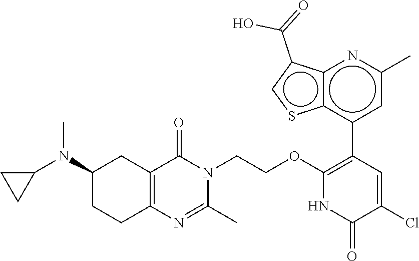 |
| 235 | 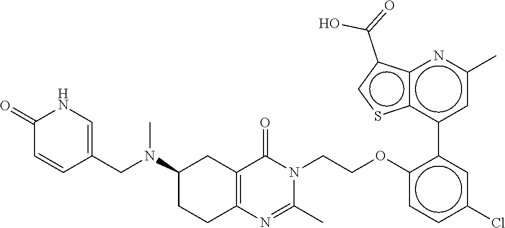 |
| 236 | 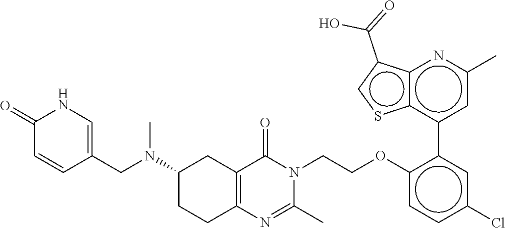 |
| 237 | 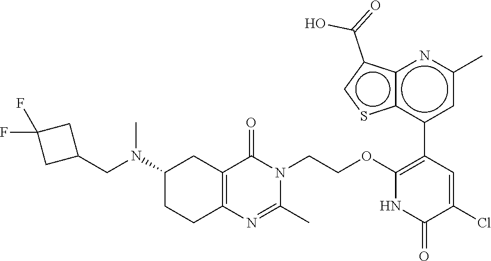 |
| 238 | 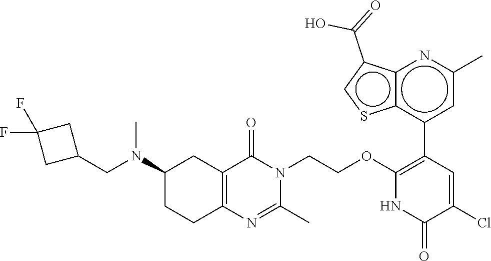 |
| 239 | 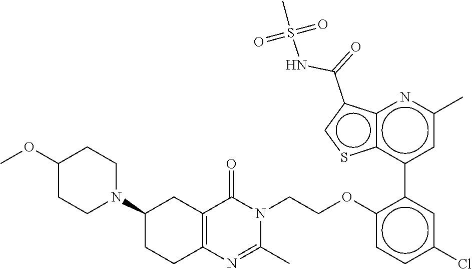 |
| 240 | 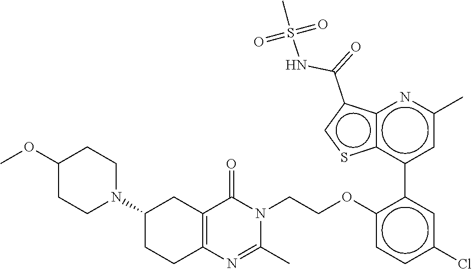 |
| 241 | 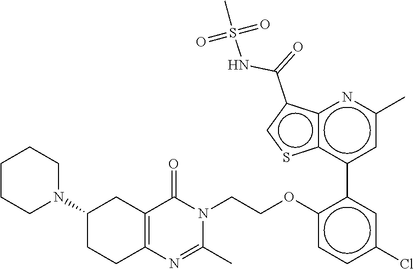 |
| 242 | 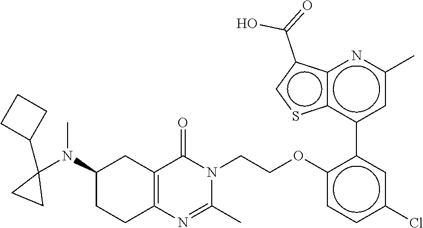 |
| 243 | 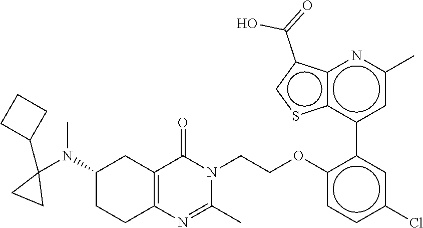 |
| 244 | 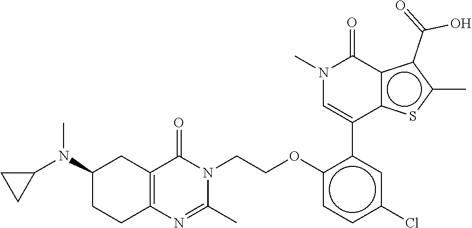 |
| 245 | 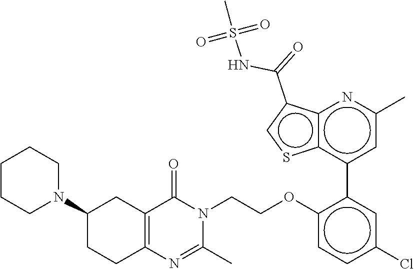 |
| 246 | 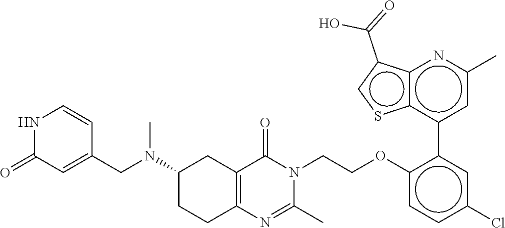 |
| 247 | 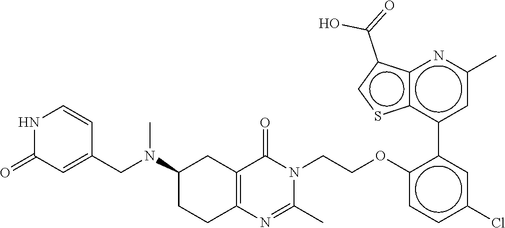 |
| 248 | 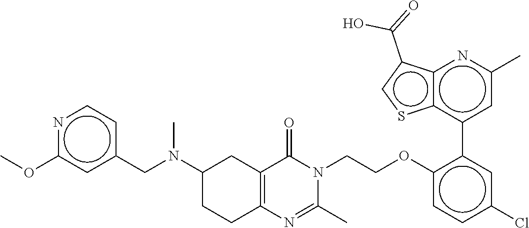 |
| 249 | 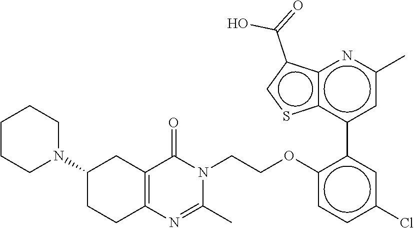 |
| 250 | 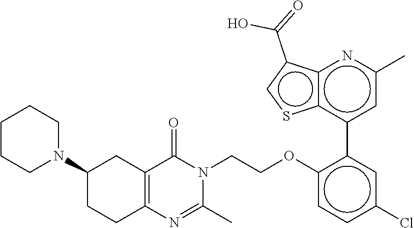 |
| 251 | 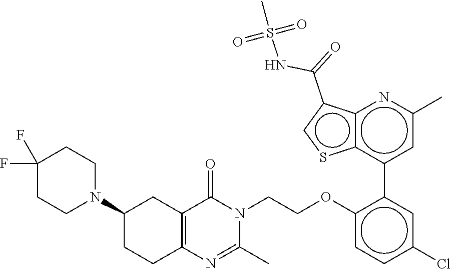 |
| 252 | 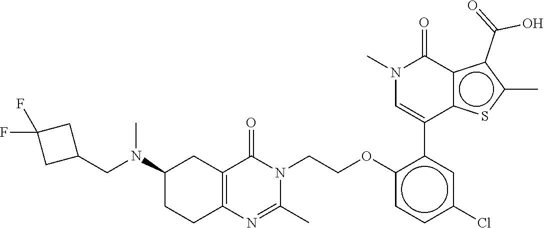 |
| 253 | 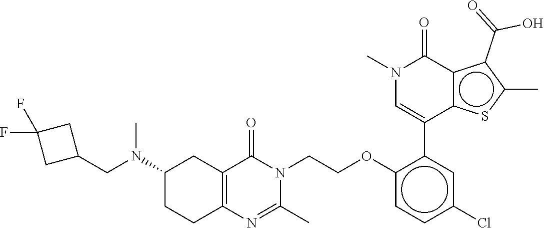 |
| 254 | 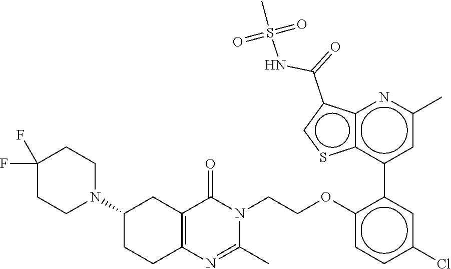 |
| 255 | 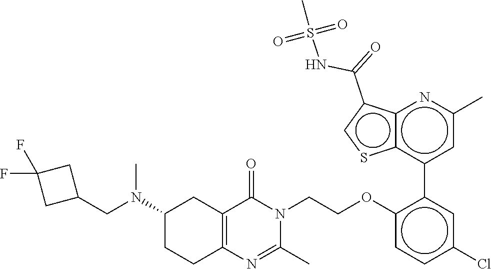 |
| 256 | 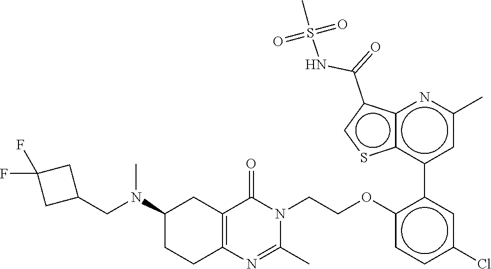 |
| 257 | 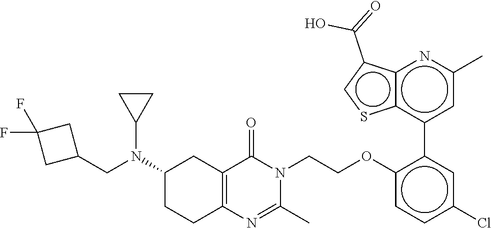 |
| 258 | 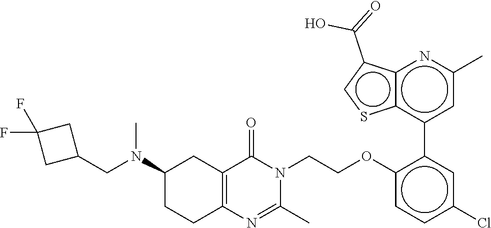 |
| 259 | 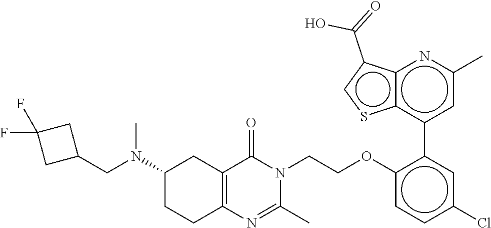 |
| 260 | 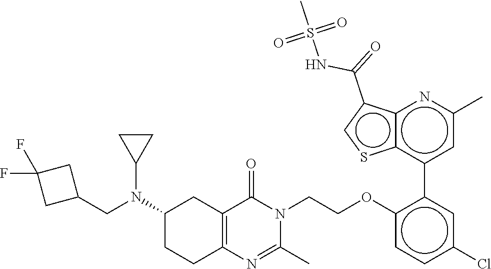 |
| 261 | 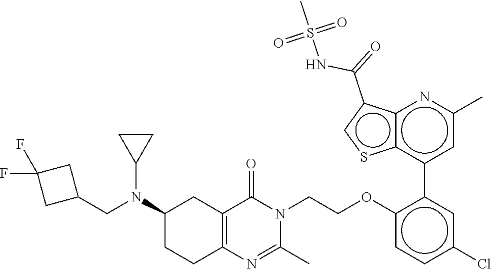 |
| 262 | 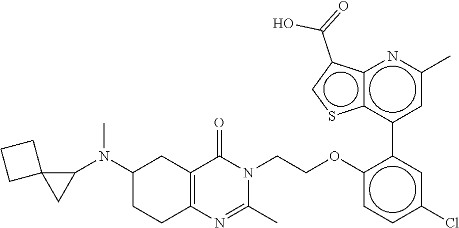 |
| 263 | 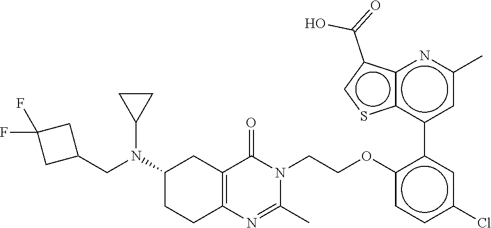 |
| 264 | 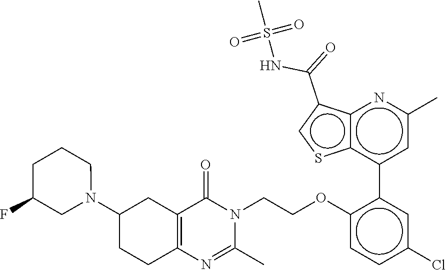 |
| 265 | 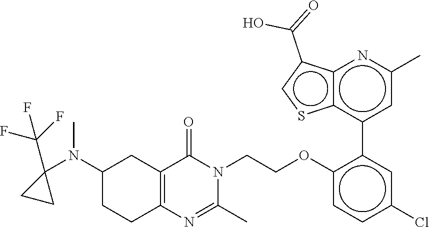 |
| 266 | 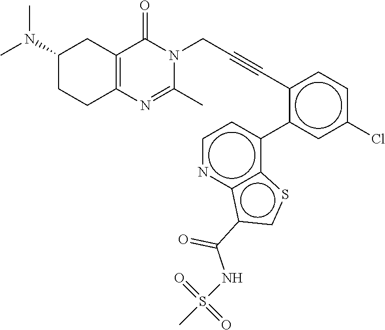 |
| 267 | 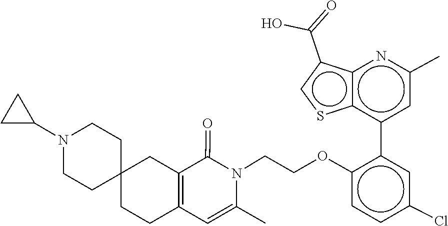 |
| 268 | 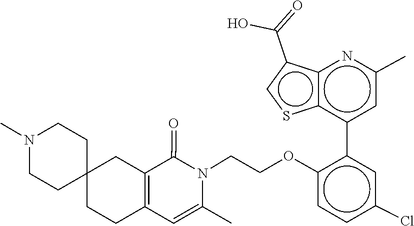 |
| 269 | 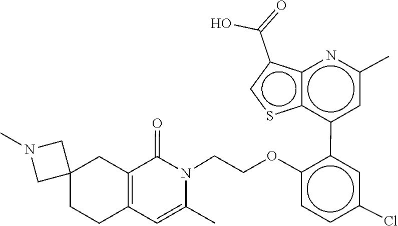 |
| 270 | 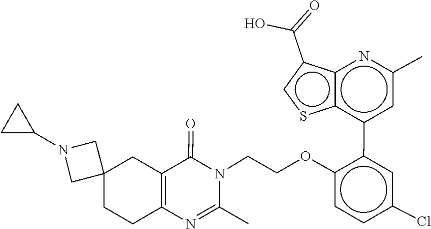 |
| 271 | 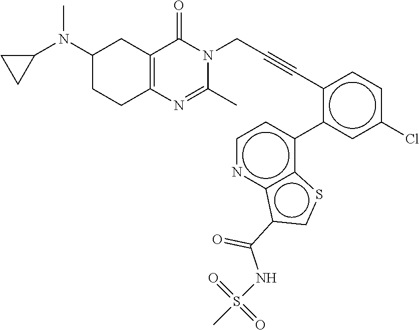 |
| 272 | 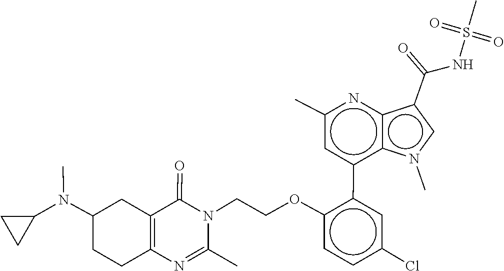 |
| 273 | 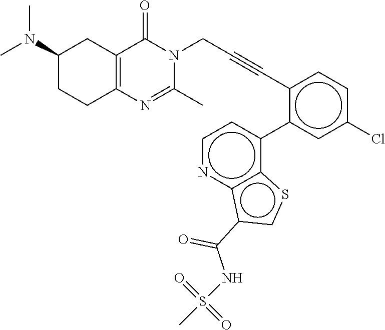 |
| 274 | 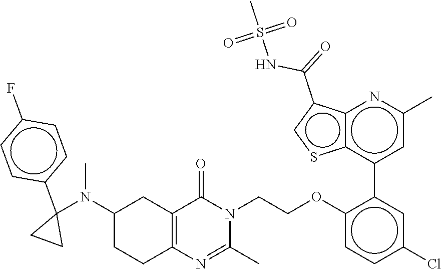 |
| 275 | 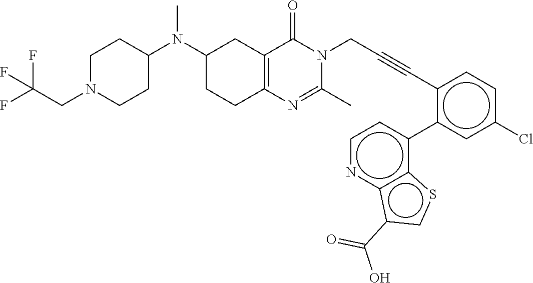 |
| 276 | 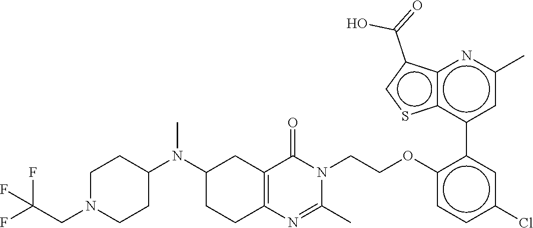 |
| 277 | 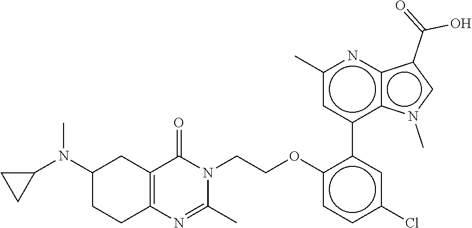 |
| 278 | 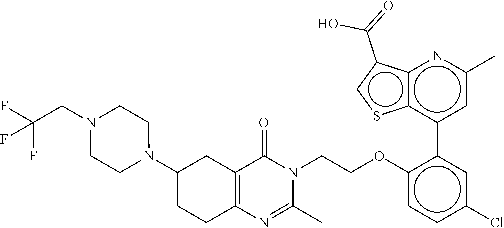 |
| 279 | 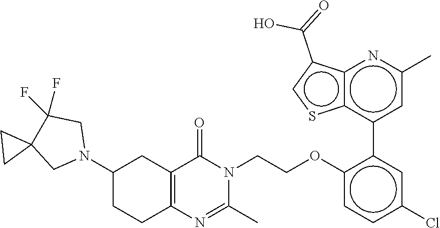 |
| 280 | 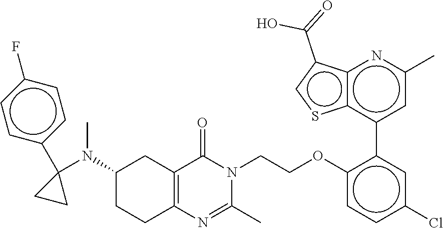 |
| 281 | 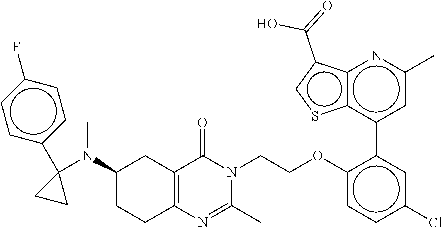 |
| 282 | 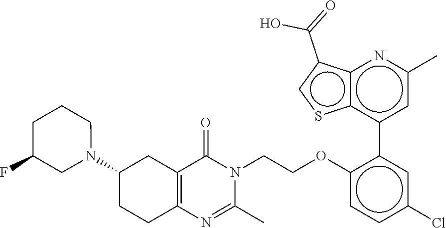 |
| 283 | 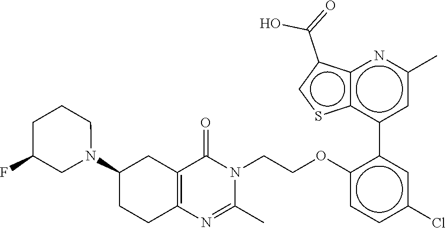 |
| 284 | 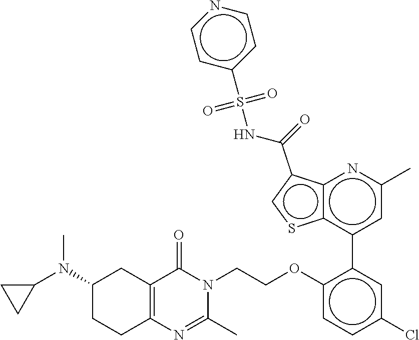 |
| 285 | 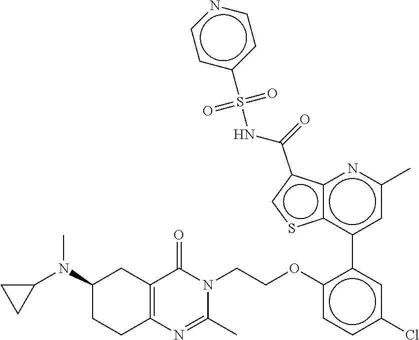 |
| 286 | 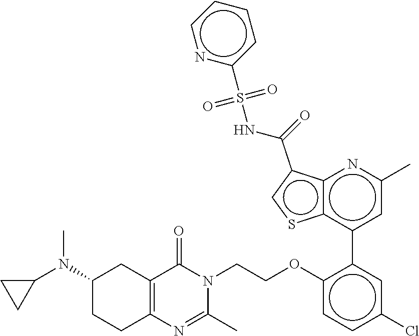 |
| 287 | 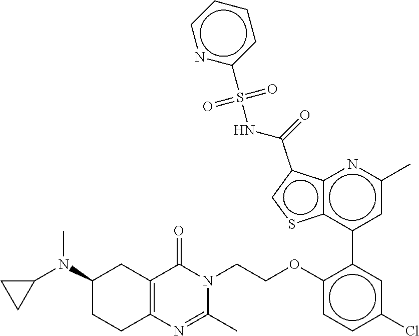 |
| 288 | 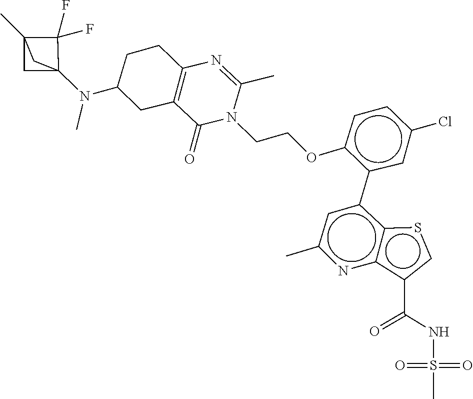 |
| 289 | 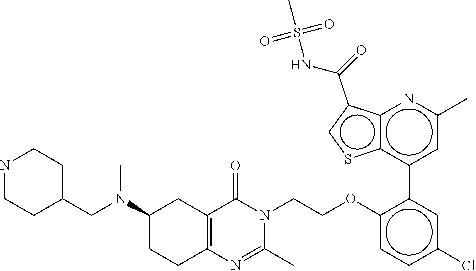 |
| 290 | 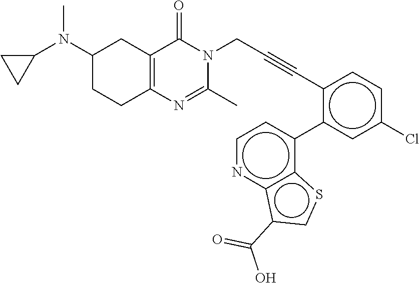 |
| 291 | 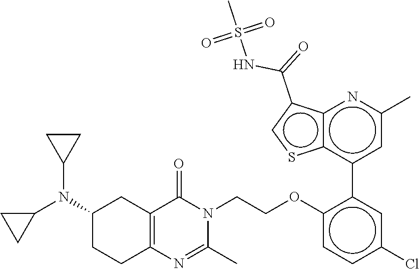 |
| 292 | 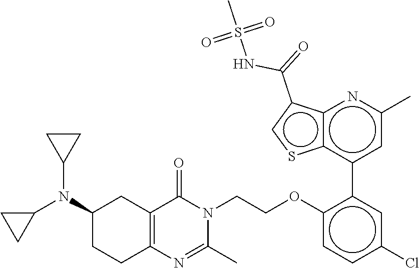 |
| 293 | 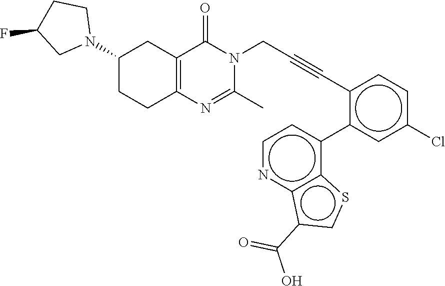 |
| 294 | 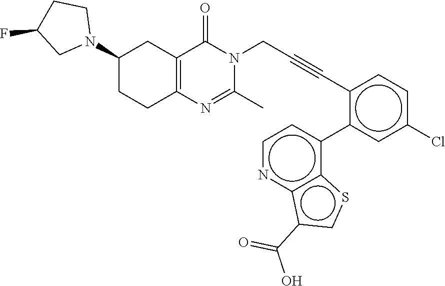 |
| 295 | 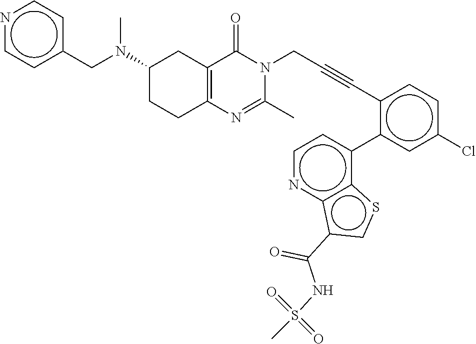 |
| 296 | 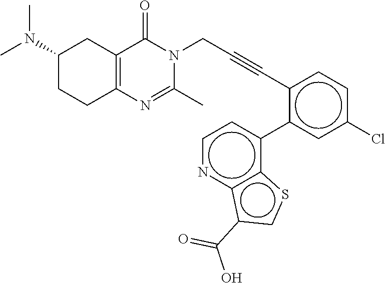 |
| 297 | 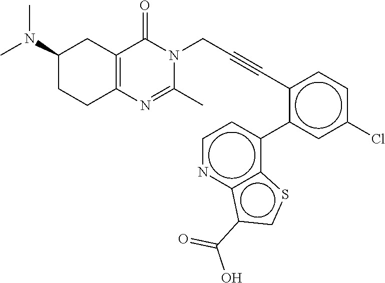 |
| 298 | 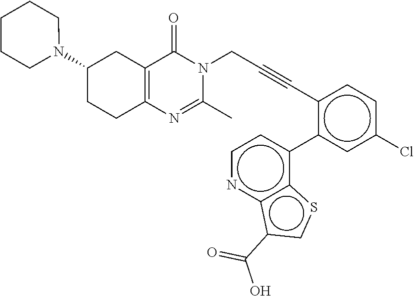 |
| 299 | 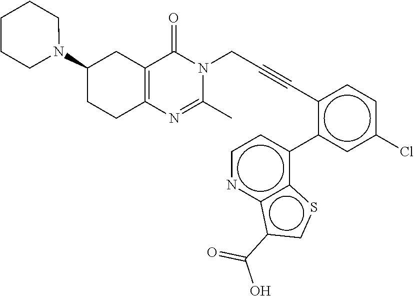 |
| 300 | 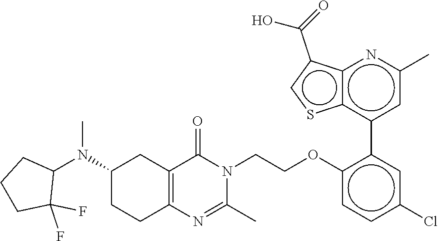 |
| 301 | 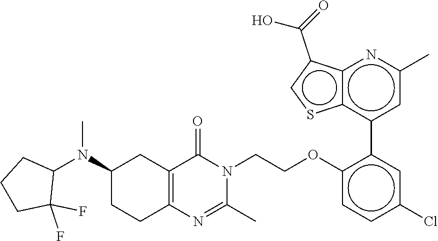 |
| 302 | 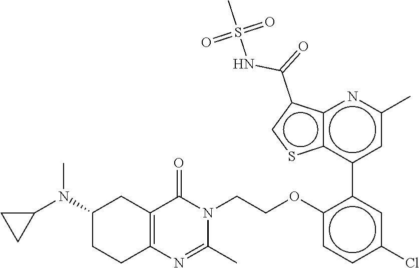 |
| 303 | 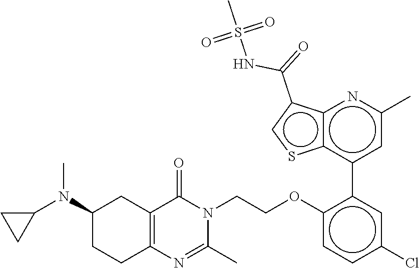 |
| 304 | 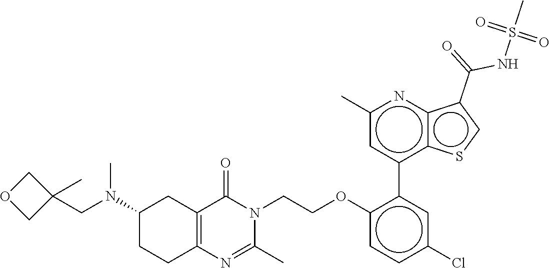 |
| 305 | 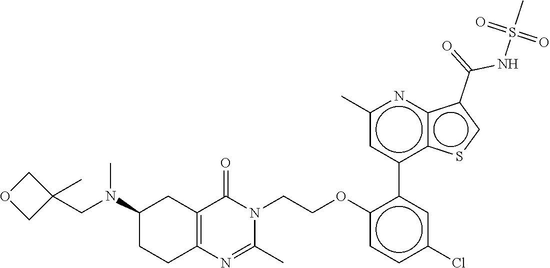 |
| 306 | 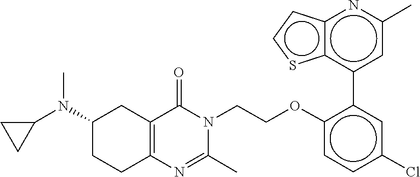 |
| 307 | 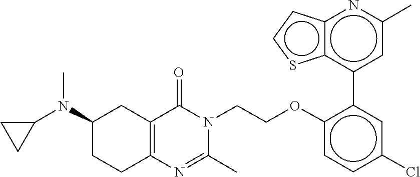 |
| 308 | 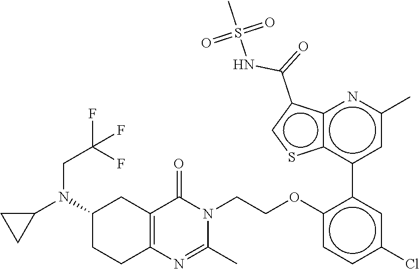 |
| 309 | 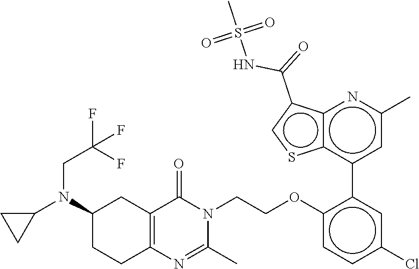 |
| 310 | 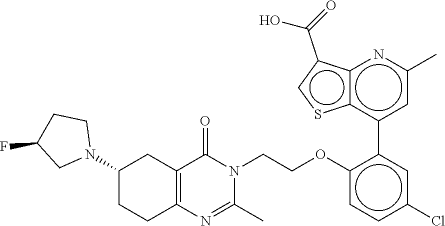 |
| 311 | 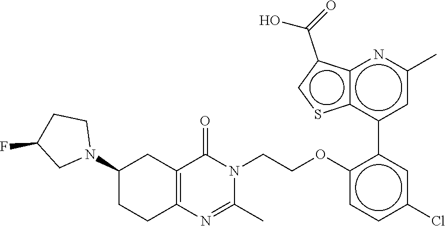 |
| 312 | 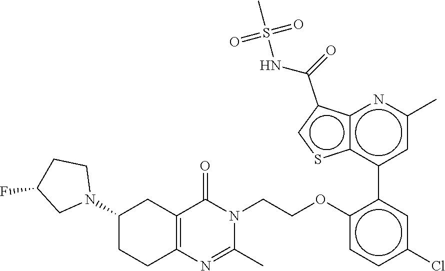 |
| 313 | 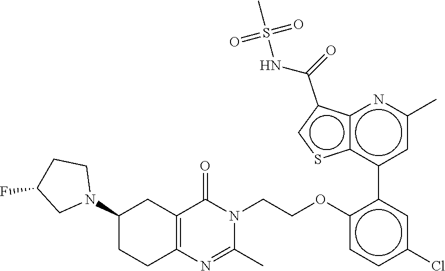 |
| 314 | 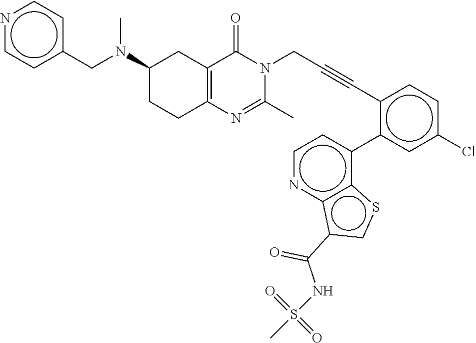 |
| 315 | 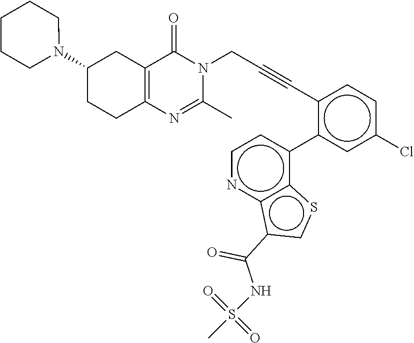 |
| 316 | 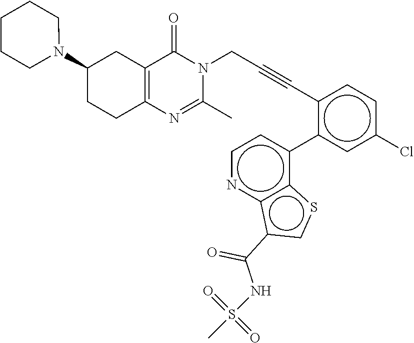 |
| 317 | 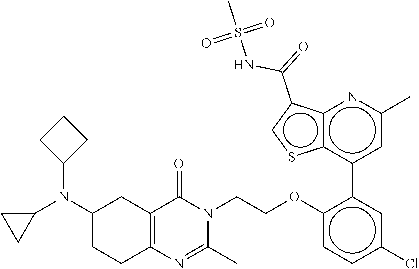 |
| 318 | 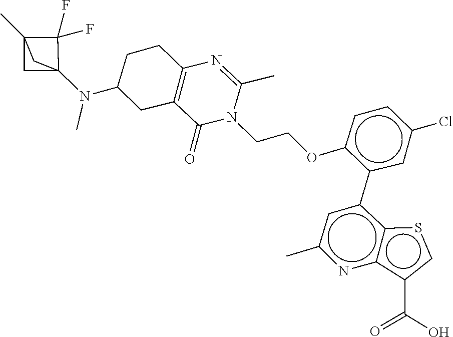 |
| 319 | 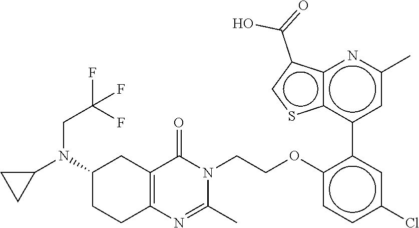 |
| 320 | 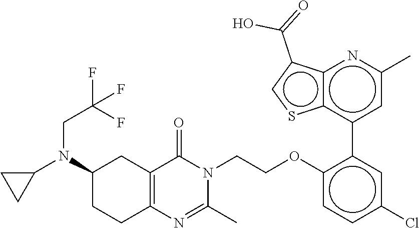 |
| 321 | 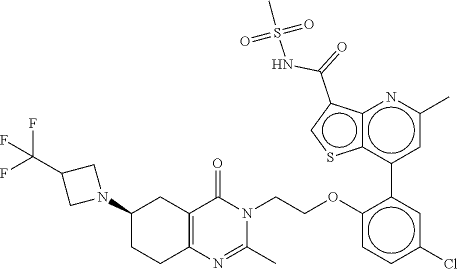 |
| 322 | 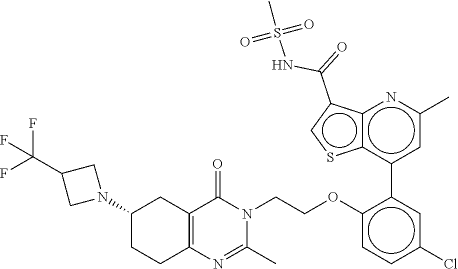 |
| 323 | 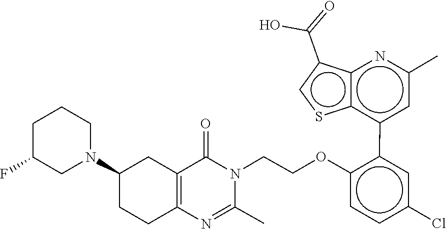 |
| 324 | 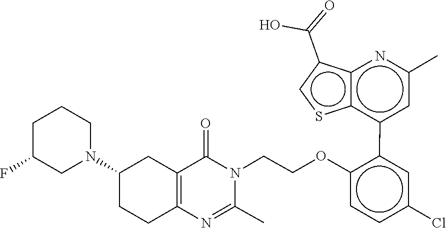 |
| 325 | 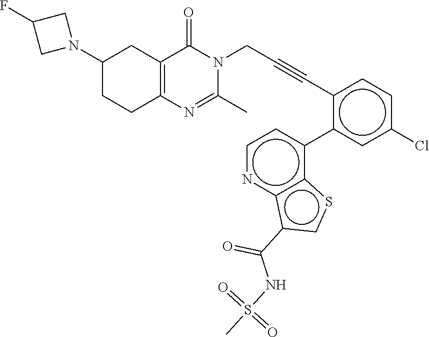 |
| 326 | 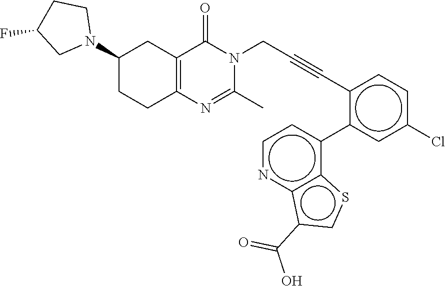 |
| 327 | 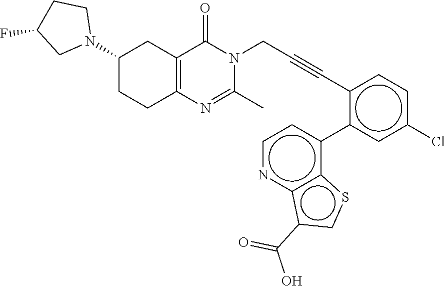 |
| 328 | 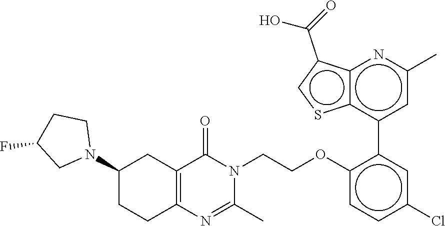 |
| 329 | 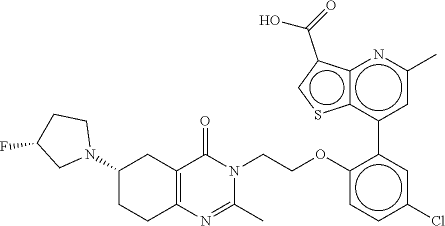 |
| 330 | 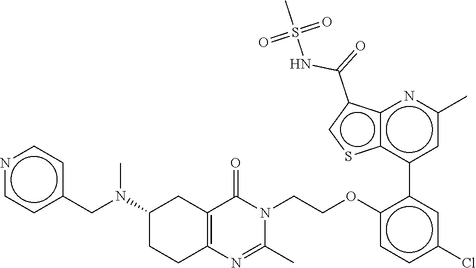 |
| 331 | 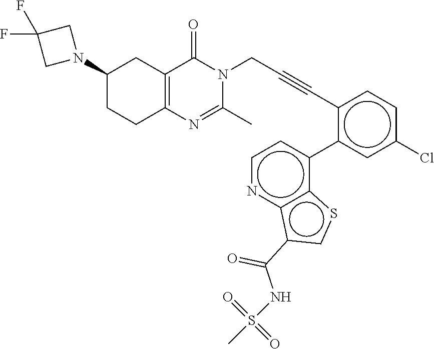 |
| 332 | 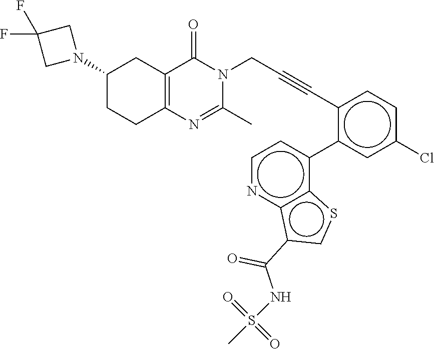 |
| 333 | 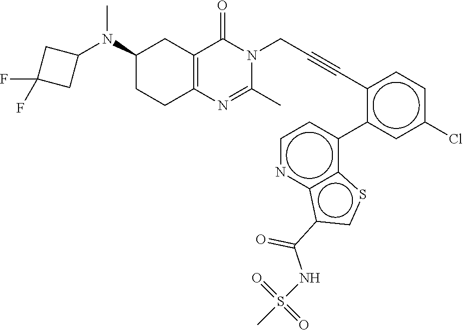 |
| 334 | 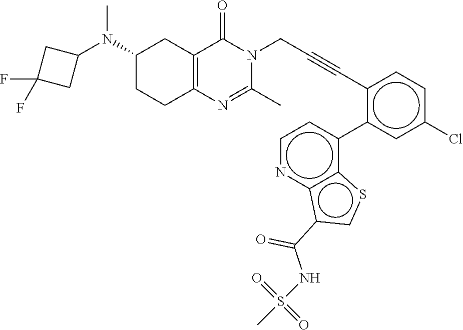 |
| 335 | 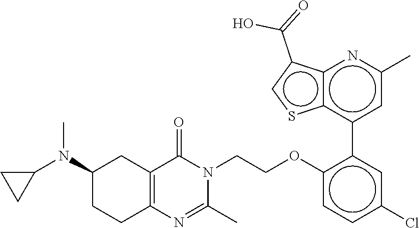 |
| 336 | 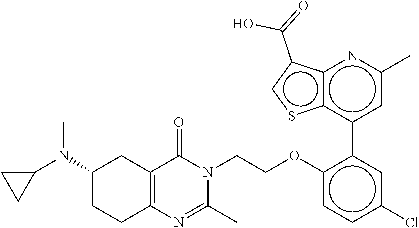 |
| 337 | 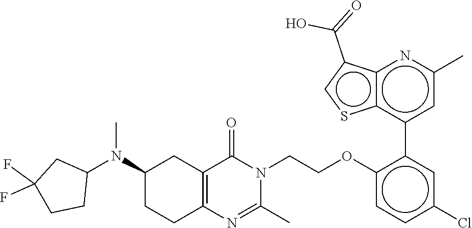 |
| 338 | 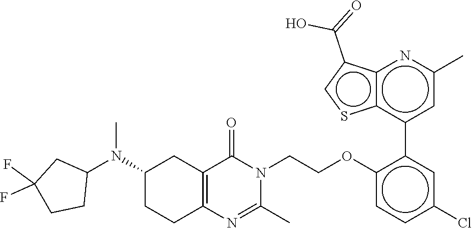 |
| 339 | 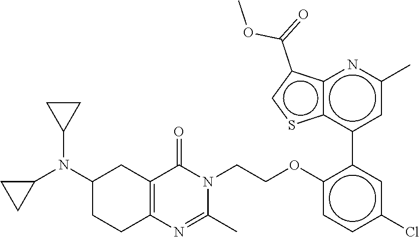 |
| 340 | 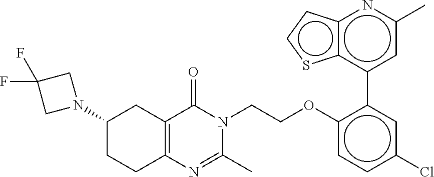 |
| 341 | 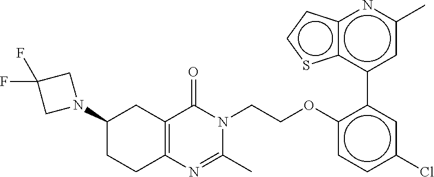 |
| 342 | 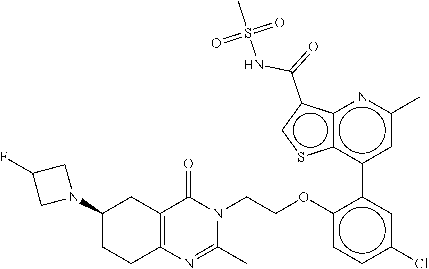 |
| 343 | 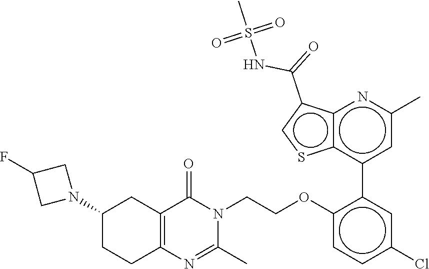 |
| 344 | 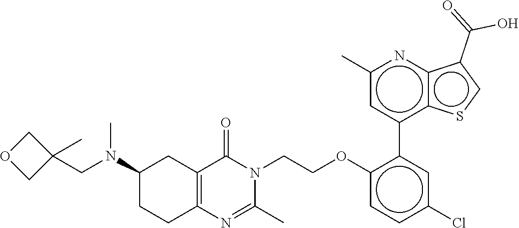 |
| 345 | 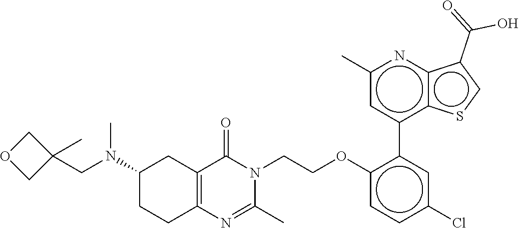 |
| 346 | 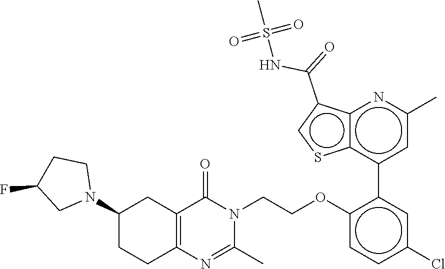 |
| 347 | 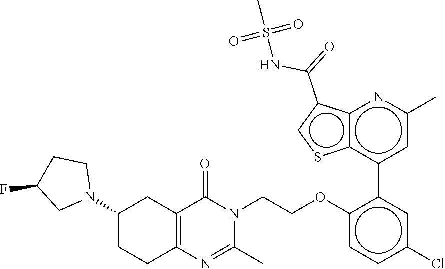 |
| 348 | 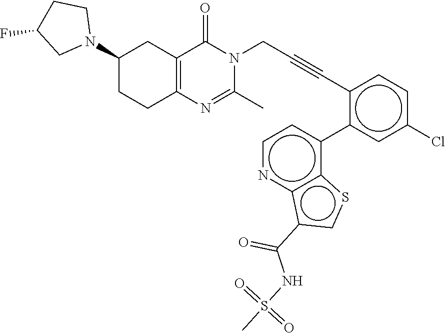 |
| 349 | 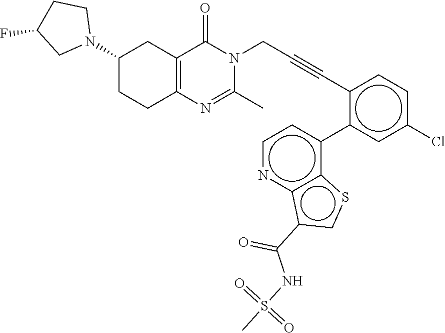 |
| 350 | 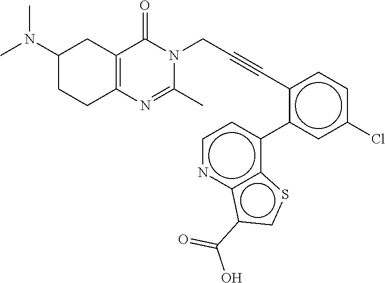 |
| 351 | 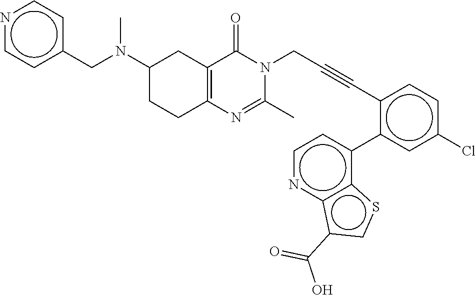 |
| 352 | 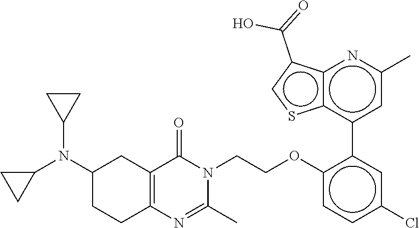 |
| 353 | 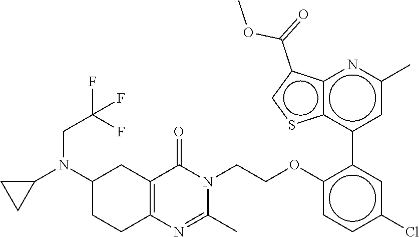 |
| 354 | 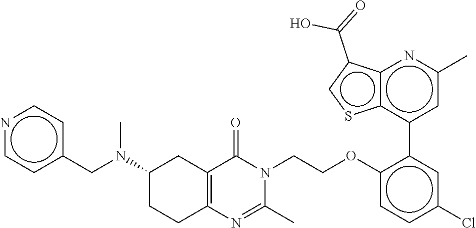 |
| 355 | 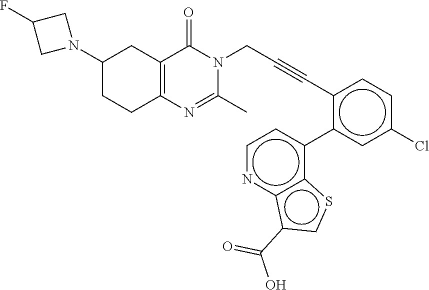 |
| 356 | 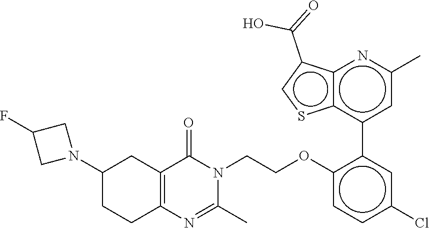 |
| 357 | 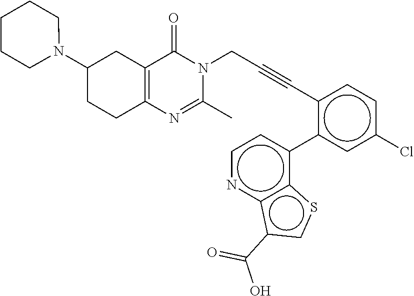 |
| 358 | 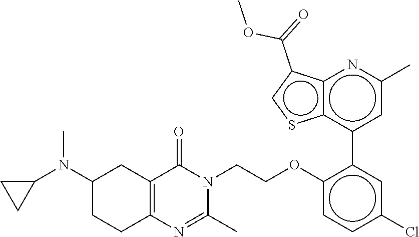 |
| 359 | 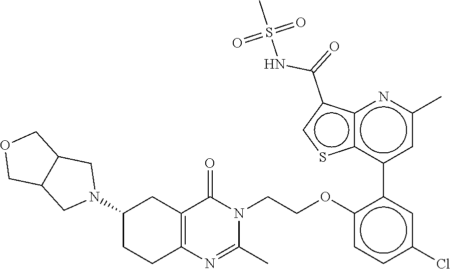 |
| 360 | 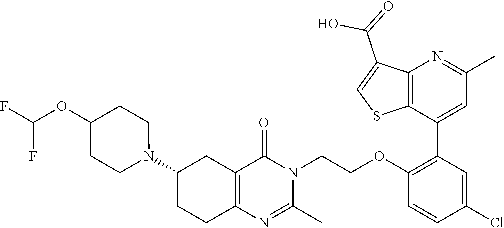 [SMILES: Cc1cc(-c2cc(Cl)ccc2OCCn2c(C)nc3c(c2=O)C[C@@H](N2CCC(OC(F)F)CC2)CC3)c2scc(C(=O)O)c2n1] |
| 361 | 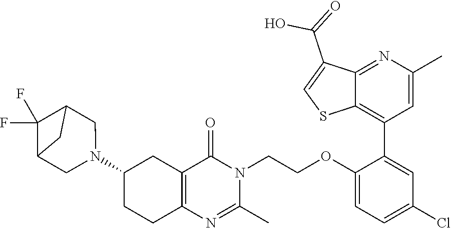 [SMILES: Cc1cc(-c2cc(Cl)ccc2OCCn2c(C)nc3c(c2=O)C[C@@H](N2CC4CC(C2)C4(F)F)CC3)c2scc(C(=O)O)c2n1] |
| 362 | 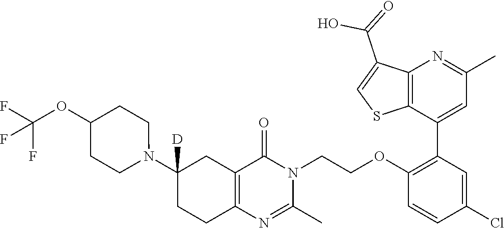 [SMILES: [2H][C@]1(N2CCC(OC(F)(F)F)CC2)CCc2nc(C)n(CCOc3ccc(Cl)cc3-c3cc(C)nc4c(C(=O)O)csc34)c(=O)c2C1] |
| 363 | 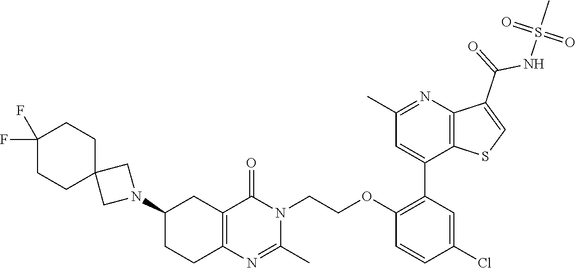 [SMILES: Cc1cc(-c2cc(Cl)ccc2OCCn2c(C)nc3c(c2=O)C[C@H](N2CC4(CCC(F)(F)CC4)C2)CC3)c2scc(C(=O)NS(C)(=O)=O)c2n1] |
| 364 | 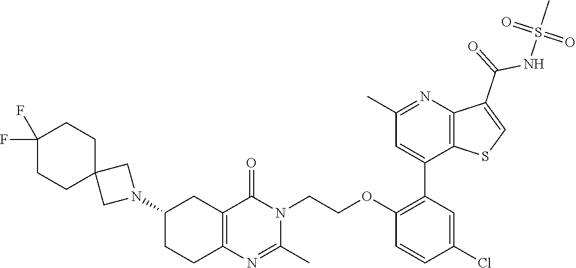 [SMILES: Cc1cc(-c2cc(Cl)ccc2OCCn2c(C)nc3c(c2=O)C[C@@H](N2CC4(CCC(F)(F)CC4)C2)CC3)c2scc(C(=O)NS(C)(=O)=O)c2n1] |
| 365 | 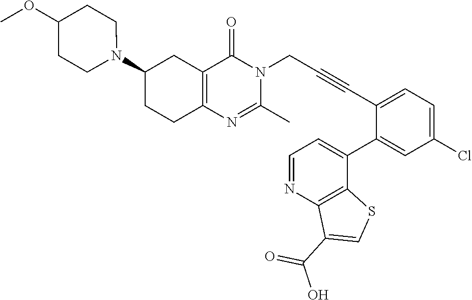 [SMILES: COC1CCN([C@@H]2CCc3nc(C)n(CC#Cc4ccc(Cl)cc4-c4ccnc5c(C(=O)O)csc45)c(=O)c3C2)CC1] |
| 366 | 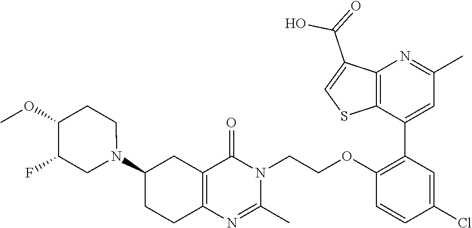 |
| 367 | 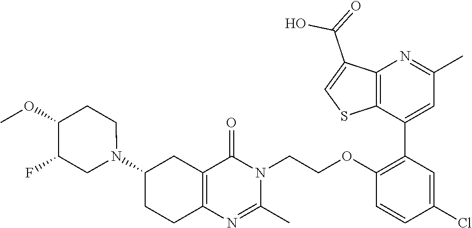 |
| 368 | 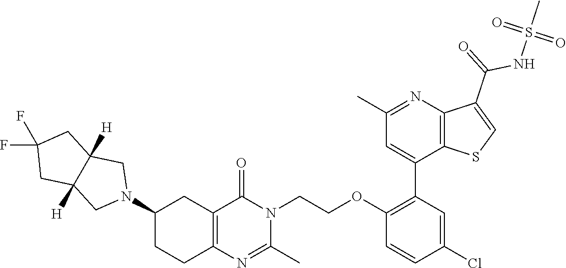 [SMILES: Cc1cc(-c2cc(Cl)ccc2OCCn2c(C)nc3c(c2=O)C[C@H](N2C[C@@H]4CC(F)(F)C[C@@H]4C2)CC3)c2scc(C(=O)NS(C)(=O)=O)c2n1] |
| 369 | 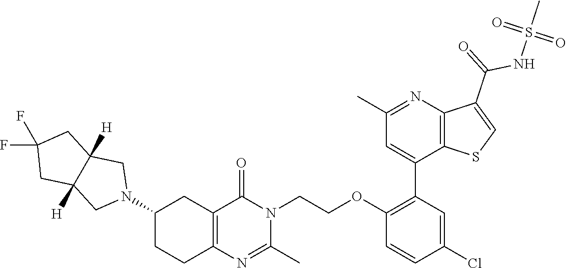 [SMILES: Cc1cc(-c2cc(Cl)ccc2OCCn2c(C)nc3c(c2=O)C[C@@H](N2C[C@@H]4CC(F)(F)C[C@@H]4C2)CC3)c2scc(C(=O)NS(C)(=O)=O)c2n1] |
| 370 | 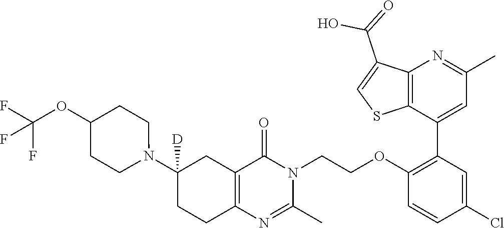 [SMILES: [2H][C@@]1(N2CCC(OC(F)(F)F)CC2)CCc2nc(C)n(CCOc3ccc(Cl)cc3-c3cc(C)nc4c(C(=O)O)csc34)c(=O)c2C1] |
| 371 | 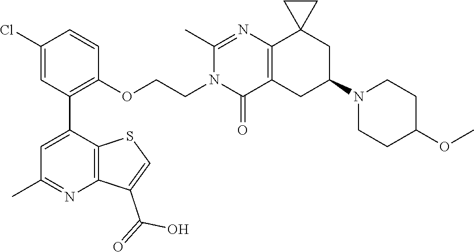 [SMILES: COC1CCN([C@H]2Cc3c(nc(C)n(CCOc4ccc(Cl)cc4-c4cc(C)nc5c(C(=O)O)csc45)c3=O)C3(CC3)C2)CC1] |
| 372 | 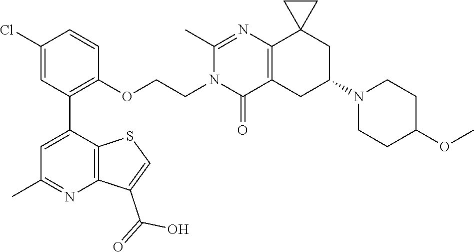 [SMILES: COC1CCN([C@@H]2Cc3c(nc(C)n(CCOc4ccc(Cl)cc4-c4cc(C)nc5c(C(=O)O)csc45)c3=O)C3(CC3)C2)CC1] |
| 373 | 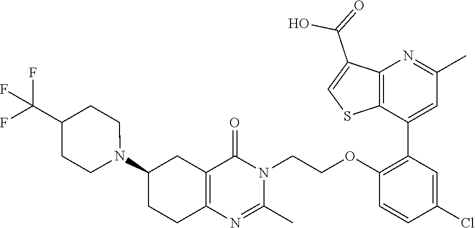 [SMILES: Cc1cc(-c2cc(Cl)ccc2OCCn2c(C)nc3c(c2=O)C[C@H](N2CCC(C(F)(F)F)CC2)CC3)c2scc(C(=O)O)c2n1] |
| 374 | 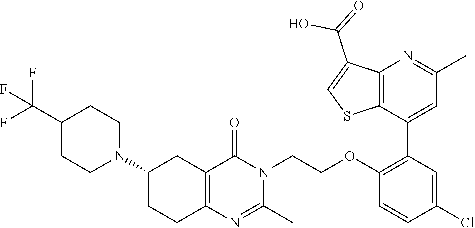 [SMILES: Cc1cc(-c2cc(Cl)ccc2OCCn2c(C)nc3c(c2=O)C[C@@H](N2CCC(C(F)(F)F)CC2)CC3)c2scc(C(=O)O)c2n1] |
| 375 | 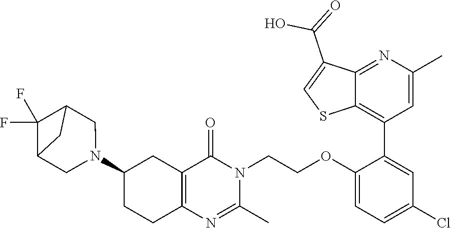 [SMILES: Cc1cc(-c2cc(Cl)ccc2OCCn2c(C)nc3c(c2=O)C[C@H](N2CC4CC(C2)C4(F)F)CC3)c2scc(C(=O)O)c2n1] |
| 376 | 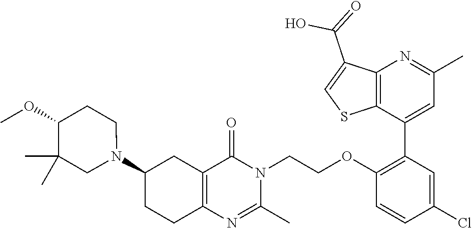 |
| 377 | 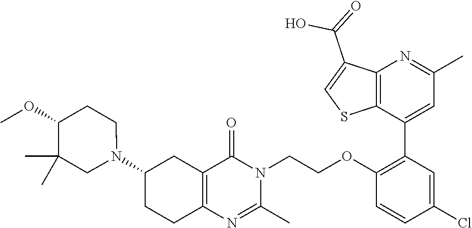 |
| 378 | 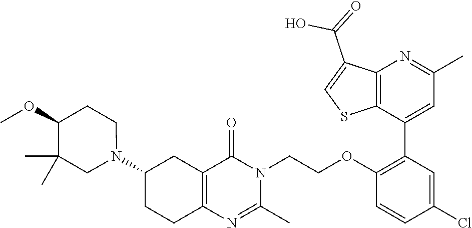 |
| 379 | 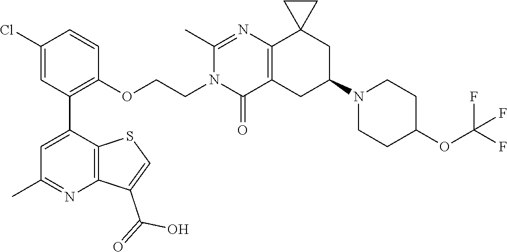 [SMILES: Cc1cc(-c2cc(Cl)ccc2OCCn2c(C)nc3c(c2=O)C[C@H](N2CCC(OC(F)(F)F)CC2)CC32CC2)c2scc(C(=O)O)c2n1] |
| 380 | 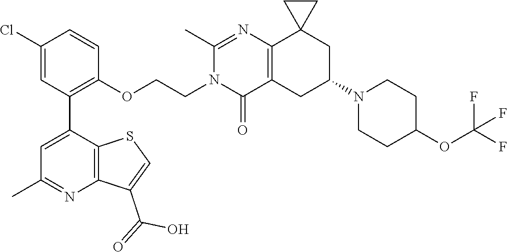 [SMILES: Cc1cc(-c2cc(Cl)ccc2OCCn2c(C)nc3c(c2=O)C[C@@H](N2CCC(OC(F)(F)F)CC2)CC32CC2)c2scc(C(=O)O)c2n1] |
| 381 | 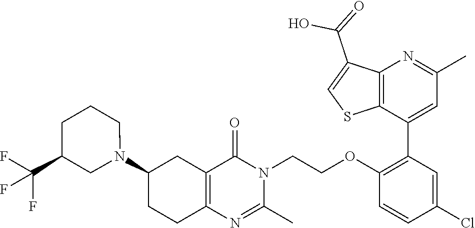 [SMILES: Cc1cc(-c2cc(Cl)ccc2OCCn2c(C)nc3c(c2=O)C[C@H](N2CCC[C@H](C(F)(F)F)C2)CC3)c2scc(C(=O)O)c2n1] |
| 382 | 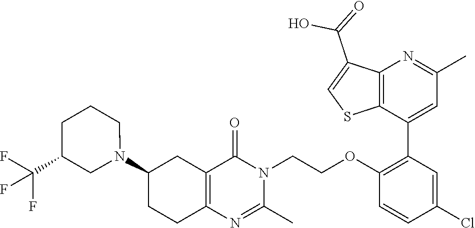 [SMILES: Cc1cc(-c2cc(Cl)ccc2OCCn2c(C)nc3c(c2=O)C[C@H](N2CCC[C@@H](C(F)(F)F)C2)CC3)c2scc(C(=O)O)c2n1] |
| 383 | 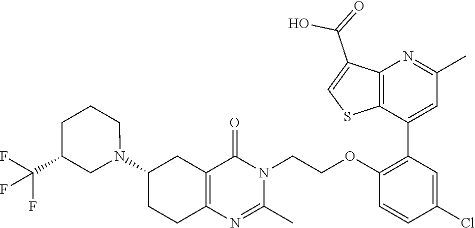 [SMILES: Cc1cc(-c2cc(Cl)ccc2OCCn2c(C)nc3c(c2=O)C[C@@H](N2CCC[C@@H](C(F)(F)F)C2)CC3)c2scc(C(=O)O)c2n1] |
| 384 | 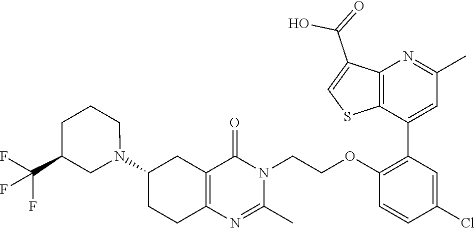 [SMILES: Cc1cc(-c2cc(Cl)ccc2OCCn2c(C)nc3c(c2=O)C[C@@H](N2CCC[C@H](C(F)(F)F)C2)CC3)c2scc(C(=O)O)c2n1] |
| 385 | 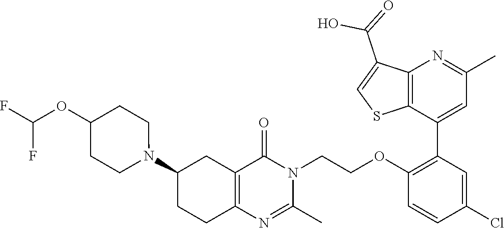 [SMILES: Cc1cc(-c2cc(Cl)ccc2OCCn2c(C)nc3c(c2=O)C[C@H](N2CCC(OC(F)F)CC2)CC3)c2scc(C(=O)O)c2n1] |
| 386 | 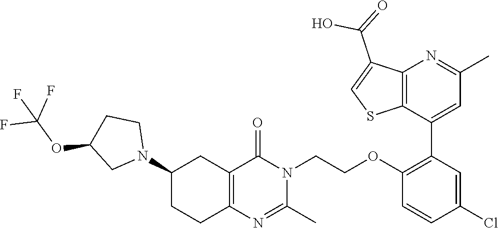 [SMILES: Cc1cc(-c2cc(Cl)ccc2OCCn2c(C)nc3c(c2=O)C[C@H](N2CC[C@H](OC(F)(F)F)C2)CC3)c2scc(C(=O)O)c2n1] |
| 387 | 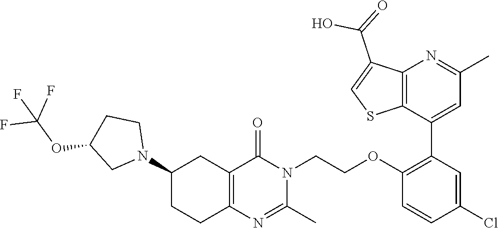 [SMILES: Cc1cc(-c2cc(Cl)ccc2OCCn2c(C)nc3c(c2=O)C[C@H](N2CC[C@@H](OC(F)(F)F)C2)CC3)c2scc(C(=O)O)c2n1] |
| 388 | 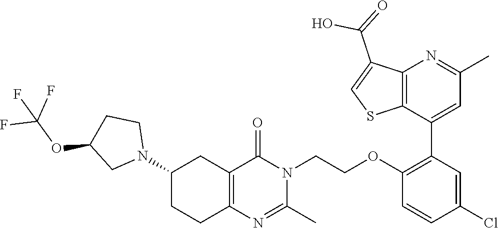 [SMILES: Cc1cc(-c2cc(Cl)ccc2OCCn2c(C)nc3c(c2=O)C[C@@H](N2CC[C@H](OC(F)(F)F)C2)CC3)c2scc(C(=O)O)c2n1] |
| 389 | 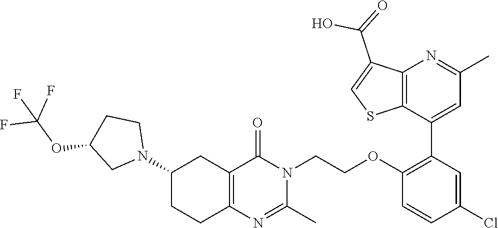 [SMILES: Cc1cc(-c2cc(Cl)ccc2OCCn2c(C)nc3c(c2=O)C[C@@H](N2CC[C@@H](OC(F)(F)F)C2)CC3)c2scc(C(=O)O)c2n1] |
| 390 | 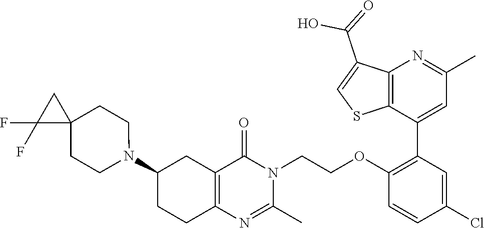 [SMILES: Cc1cc(-c2cc(Cl)ccc2OCCn2c(C)nc3c(c2=O)C[C@H](N2CCC4(CC2)CC4(F)F)CC3)c2scc(C(=O)O)c2n1] |
| 391 | 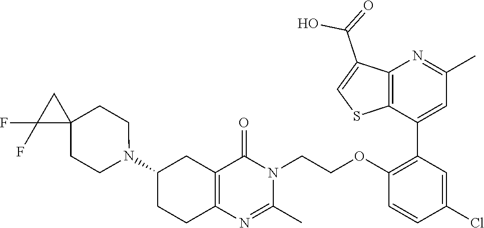 [SMILES: Cc1cc(-c2cc(Cl)ccc2OCCn2c(C)nc3c(c2=O)C[C@@H](N2CCC4(CC2)CC4(F)F)CC3)c2scc(C(=O)O)c2n1] |
| 392 | 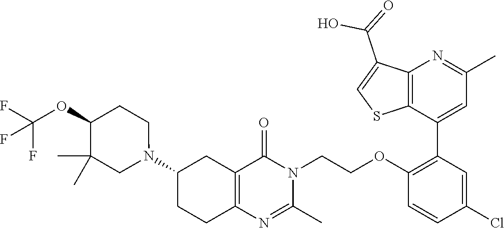 [SMILES: Cc1cc(-c2cc(Cl)ccc2OCCn2c(C)nc3c(c2=O)C[C@@H](N2CC[C@H](OC(F)(F)F)C(C)(C)C2)CC3)c2scc(C(=O)O)c2n1] |
| 393 | 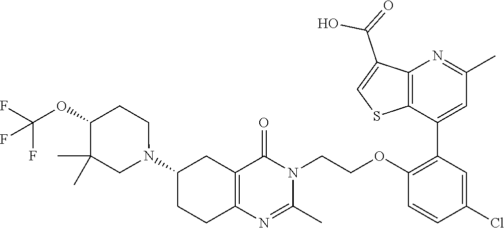 [SMILES: Cc1cc(-c2cc(Cl)ccc2OCCn2c(C)nc3c(c2=O)C[C@@H](N2CC[C@@H](OC(F)(F)F)C(C)(C)C2)CC3)c2scc(C(=O)O)c2n1] |
| 394 | 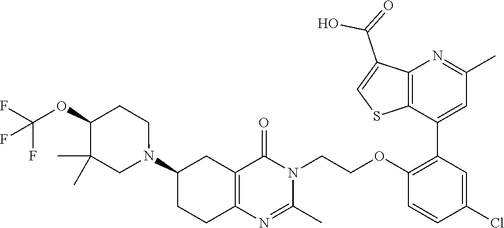 [SMILES: Cc1cc(-c2cc(Cl)ccc2OCCn2c(C)nc3c(c2=O)C[C@H](N2CC[C@H](OC(F)(F)F)C(C)(C)C2)CC3)c2scc(C(=O)O)c2n1] |
| 395 | 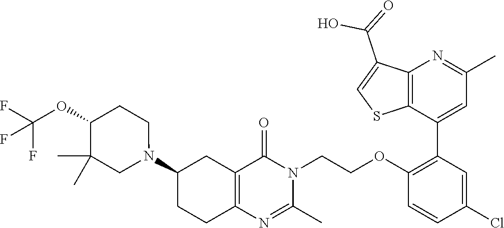 [SMILES: Cc1cc(-c2cc(Cl)ccc2OCCn2c(C)nc3c(c2=O)C[C@H](N2CC[C@@H](OC(F)(F)F)C(C)(C)C2)CC3)c2scc(C(=O)O)c2n1] |
| 396 | 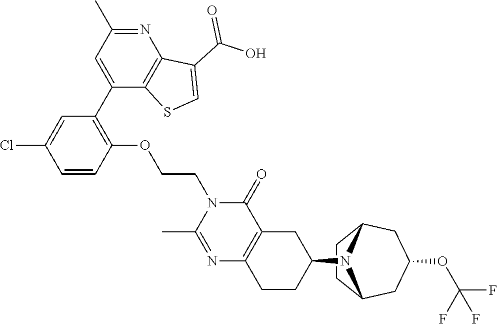 [SMILES: Cc1cc(-c2cc(Cl)ccc2OCCn2c(C)nc3c(c2=O)C[C@@H](N2[C@@H]4CC[C@H]2C[C@@H](OC(F)(F)F)C4)CC3)c2scc(C(=O)O)c2n1] |
| 397 | 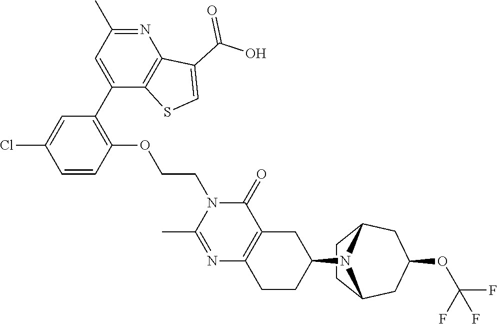 [SMILES: Cc1cc(-c2cc(Cl)ccc2OCCn2c(C)nc3c(c2=O)C[C@@H](N2[C@@H]4CC[C@H]2C[C@H](OC(F)(F)F)C4)CC3)c2scc(C(=O)O)c2n1] |
| 398 | 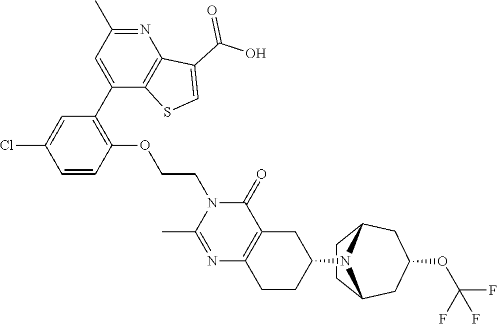 [SMILES: Cc1cc(-c2cc(Cl)ccc2OCCn2c(C)nc3c(c2=O)C[C@H](N2[C@@H]4CC[C@H]2C[C@@H](OC(F)(F)F)C4)CC3)c2scc(C(=O)O)c2n1] |
| 399 | 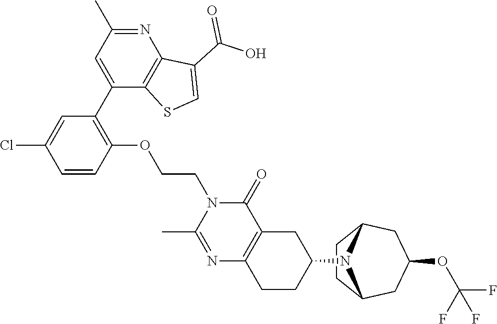 [SMILES: Cc1cc(-c2cc(Cl)ccc2OCCn2c(C)nc3c(c2=O)C[C@H](N2[C@@H]4CC[C@H]2C[C@H](OC(F)(F)F)C4)CC3)c2scc(C(=O)O)c2n1] |
| 400 | 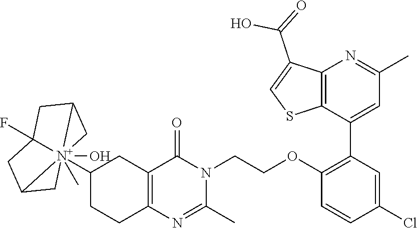 |
| 401 | 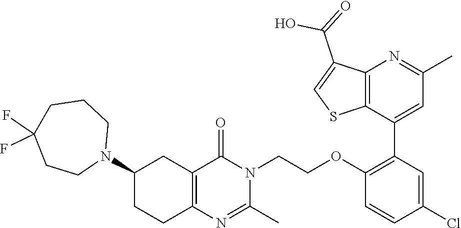 [SMILES: Cc1cc(-c2cc(Cl)ccc2OCCn2c(C)nc3c(c2=O)C[C@H](N2CCCC(F)(F)CC2)CC3)c2scc(C(=O)O)c2n1] |
| 402 | 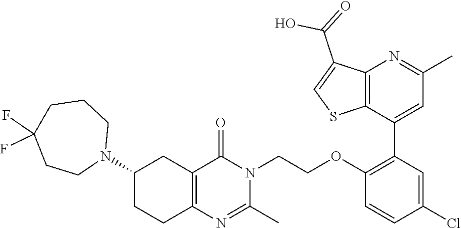 [SMILES: Cc1cc(-c2cc(Cl)ccc2OCCn2c(C)nc3c(c2=O)C[C@@H](N2CCCC(F)(F)CC2)CC3)c2scc(C(=O)O)c2n1] |
| 403 | 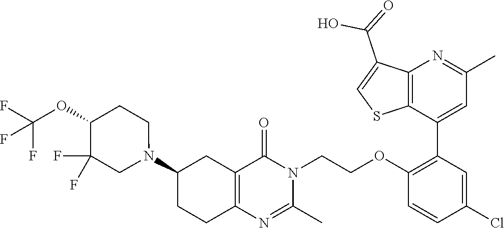 [SMILES: Cc1cc(-c2cc(Cl)ccc2OCCn2c(C)nc3c(c2=O)C[C@H](N2CC[C@@H](OC(F)(F)F)C(F)(F)C2)CC3)c2scc(C(=O)O)c2n1] |
| 404 | 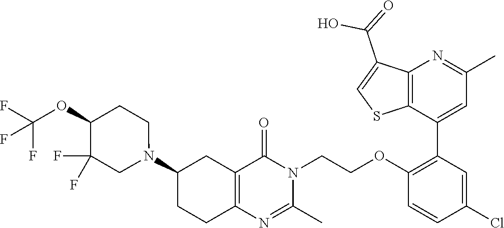 [SMILES: Cc1cc(-c2cc(Cl)ccc2OCCn2c(C)nc3c(c2=O)C[C@H](N2CC[C@H](OC(F)(F)F)C(F)(F)C2)CC3)c2scc(C(=O)O)c2n1] |
| 405 | 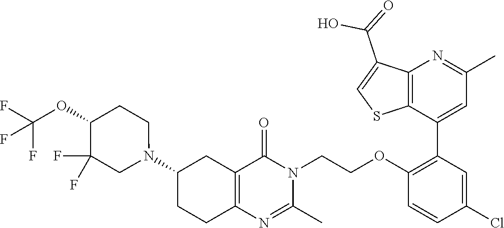 [SMILES: Cc1cc(-c2cc(Cl)ccc2OCCn2c(C)nc3c(c2=O)C[C@@H](N2CC[C@@H](OC(F)(F)F)C(F)(F)C2)CC3)c2scc(C(=O)O)c2n1] |
| 406 | 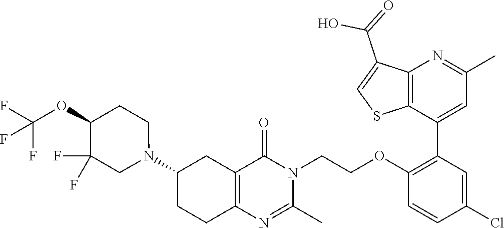 [SMILES: Cc1cc(-c2cc(Cl)ccc2OCCn2c(C)nc3c(c2=O)C[C@@H](N2CC[C@H](OC(F)(F)F)C(F)(F)C2)CC3)c2scc(C(=O)O)c2n1] |
| 407 | 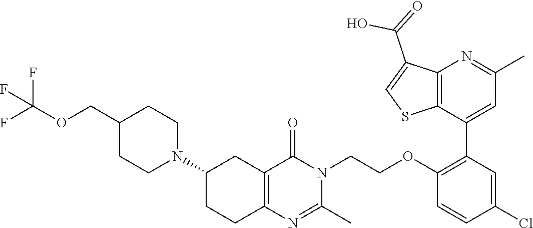 [SMILES: Cc1cc(-c2cc(Cl)ccc2OCCn2c(C)nc3c(c2=O)C[C@@H](N2CCC(COC(F)(F)F)CC2)CC3)c2scc(C(=O)O)c2n1] |
| 408 | 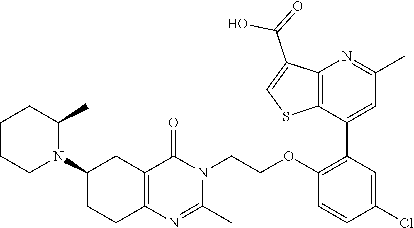 [SMILES: Cc1cc(-c2cc(Cl)ccc2OCCn2c(C)nc3c(c2=O)C[C@H](N2CCCC[C@H]2C)CC3)c2scc(C(=O)O)c2n1] |
| 409 | 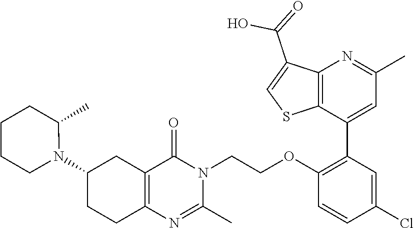 [SMILES: Cc1cc(-c2cc(Cl)ccc2OCCn2c(C)nc3c(c2=O)C[C@@H](N2CCCC[C@@H]2C)CC3)c2scc(C(=O)O)c2n1] |
| 410 | 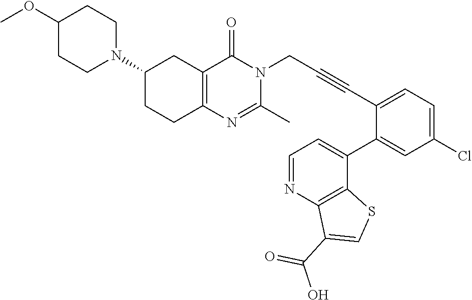 [SMILES: COC1CCN([C@H]2CCc3nc(C)n(CC#Cc4ccc(Cl)cc4-c4ccnc5c(C(=O)O)csc45)c(=O)c3C2)CC1] |
| 411 | 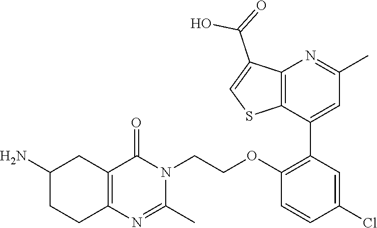 [SMILES: Cc1cc(-c2cc(Cl)ccc2OCCn2c(C)nc3c(c2=O)CC(N)CC3)c2scc(C(=O)O)c2n1] |
| 412 | 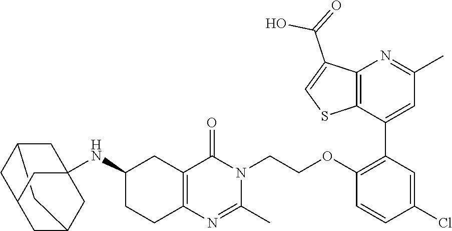 [SMILES: Cc1cc(-c2cc(Cl)ccc2OCCn2c(C)nc3c(c2=O)C[C@H](NC24CC5CC(CC(C5)C2)C4)CC3)c2scc(C(=O)O)c2n1] |
| 413 | 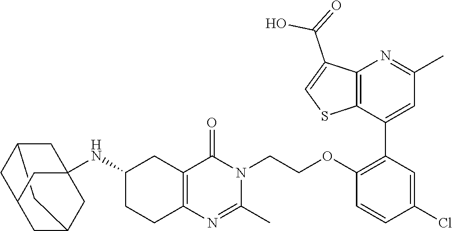 [SMILES: Cc1cc(-c2cc(Cl)ccc2OCCn2c(C)nc3c(c2=O)C[C@@H](NC24CC5CC(CC(C5)C2)C4)CC3)c2scc(C(=O)O)c2n1] |
| 414 | 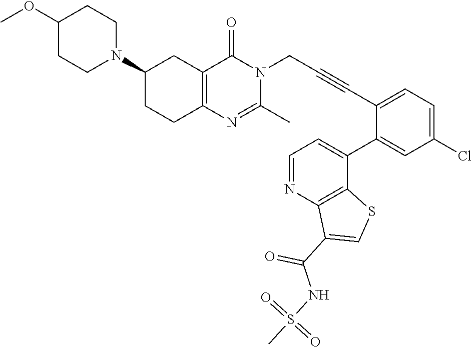 [SMILES: COC1CCN([C@@H]2CCc3nc(C)n(CC#Cc4ccc(Cl)cc4-c4ccnc5c(C(=O)NS(C)(=O)=O)csc45)c(=O)c3C2)CC1] |
| 415 | 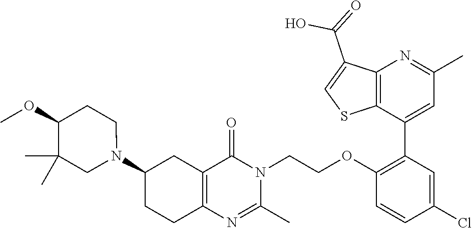 |
| 416 | 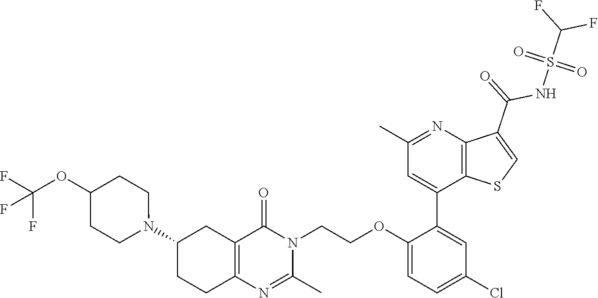 [SMILES: Cc1cc(-c2cc(Cl)ccc2OCCn2c(C)nc3c(c2=O)C[C@@H](N2CCC(OC(F)(F)F)CC2)CC3)c2scc(C(=O)NS(=O)(=O)C(F)F)c2n1] |
| 417 | 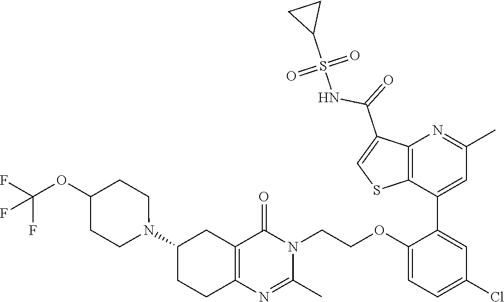 [SMILES: Cc1cc(-c2cc(Cl)ccc2OCCn2c(C)nc3c(c2=O)C[C@@H](N2CCC(OC(F)(F)F)CC2)CC3)c2scc(C(=O)NS(=O)(=O)C3CC3)c2n1] |
| 418 | 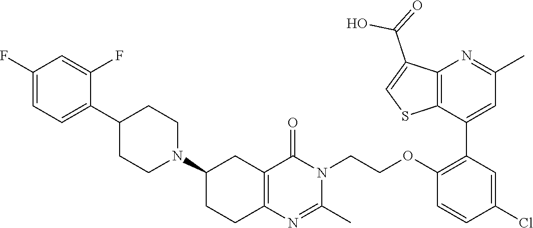 [SMILES: Cc1cc(-c2cc(Cl)ccc2OCCn2c(C)nc3c(c2=O)C[C@H](N2CCC(c4ccc(F)cc4F)CC2)CC3)c2scc(C(=O)O)c2n1] |
| 419 | 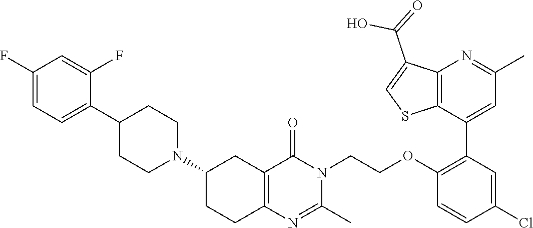 [SMILES: Cc1cc(-c2cc(Cl)ccc2OCCn2c(C)nc3c(c2=O)C[C@@H](N2CCC(c4ccc(F)cc4F)CC2)CC3)c2scc(C(=O)O)c2n1] |
| 420 | 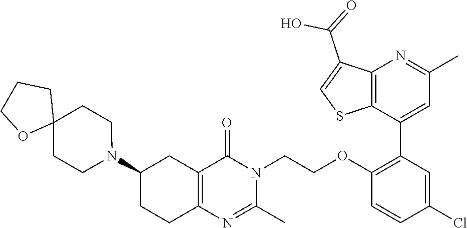 [SMILES: Cc1cc(-c2cc(Cl)ccc2OCCn2c(C)nc3c(c2=O)C[C@H](N2CCC4(CCCO4)CC2)CC3)c2scc(C(=O)O)c2n1] |
| 421 | 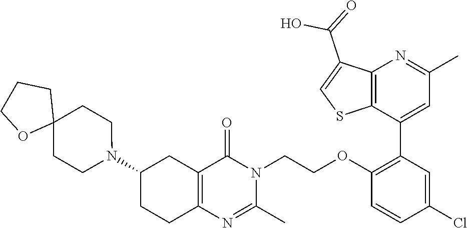 [SMILES: Cc1cc(-c2cc(Cl)ccc2OCCn2c(C)nc3c(c2=O)C[C@@H](N2CCC4(CCCO4)CC2)CC3)c2scc(C(=O)O)c2n1] |
| 422 | 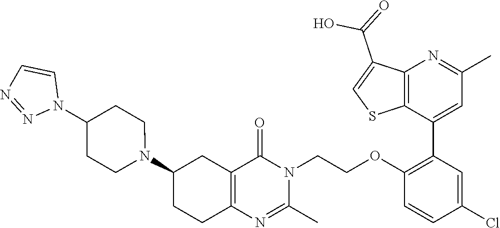 [SMILES: Cc1cc(-c2cc(Cl)ccc2OCCn2c(C)nc3c(c2=O)C[C@H](N2CCC(n4ccnn4)CC2)CC3)c2scc(C(=O)O)c2n1] |
| 423 | 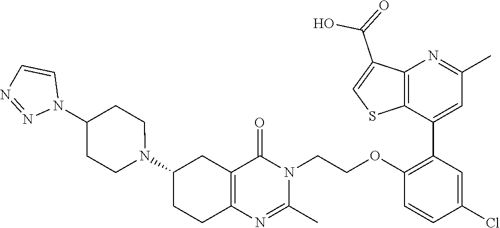 [SMILES: Cc1cc(-c2cc(Cl)ccc2OCCn2c(C)nc3c(c2=O)C[C@@H](N2CCC(n4ccnn4)CC2)CC3)c2scc(C(=O)O)c2n1] |
| 424 | 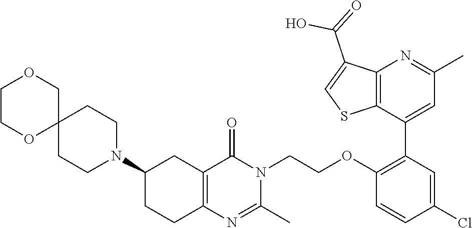 [SMILES: Cc1cc(-c2cc(Cl)ccc2OCCn2c(C)nc3c(c2=O)C[C@H](N2CCC4(CC2)COCCO4)CC3)c2scc(C(=O)O)c2n1] |
| 425 | 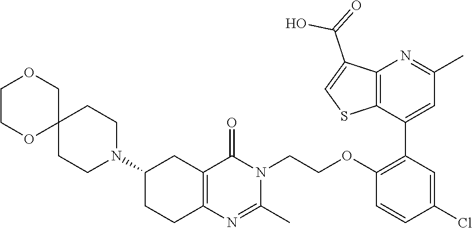 [SMILES: Cc1cc(-c2cc(Cl)ccc2OCCn2c(C)nc3c(c2=O)C[C@@H](N2CCC4(CC2)COCCO4)CC3)c2scc(C(=O)O)c2n1] |
| 426 | 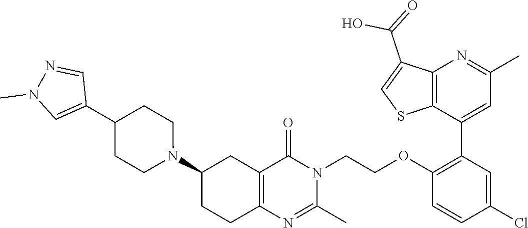 |
| 427 | 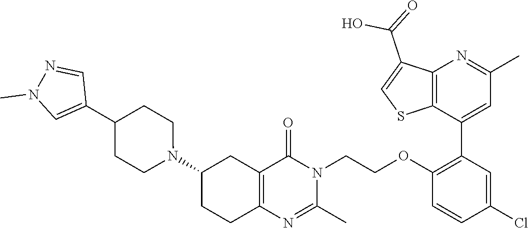 |
| 428 | 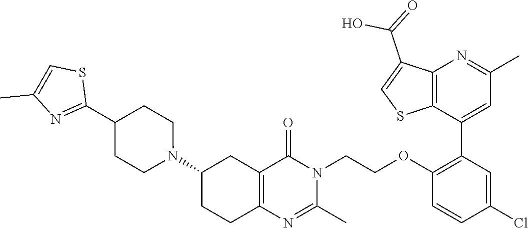 |
| 429 | 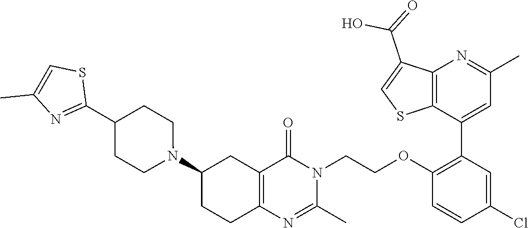 |
| 430 | 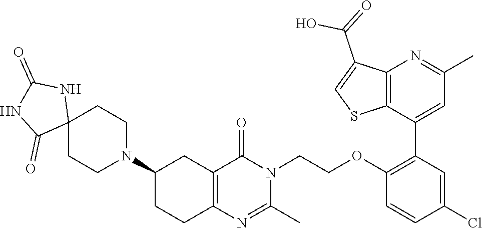 [SMILES: Cc1cc(-c2cc(Cl)ccc2OCCn2c(C)nc3c(c2=O)C[C@H](N2CCC4(CC2)NC(=O)NC4=O)CC3)c2scc(C(=O)O)c2n1] |
| 431 | 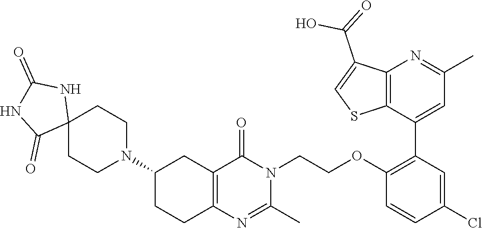 [SMILES: Cc1cc(-c2cc(Cl)ccc2OCCn2c(C)nc3c(c2=O)C[C@@H](N2CCC4(CC2)NC(=O)NC4=O)CC3)c2scc(C(=O)O)c2n1] |
| 432 | 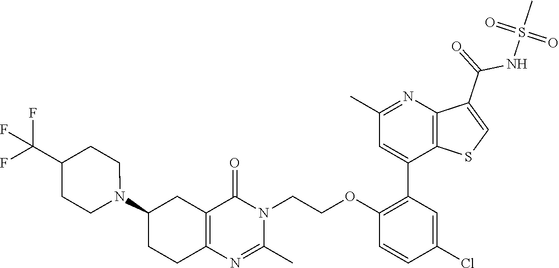 [SMILES: Cc1cc(-c2cc(Cl)ccc2OCCn2c(C)nc3c(c2=O)C[C@H](N2CCC(C(F)(F)F)CC2)CC3)c2scc(C(=O)NS(C)(=O)=O)c2n1] |
| 433 | 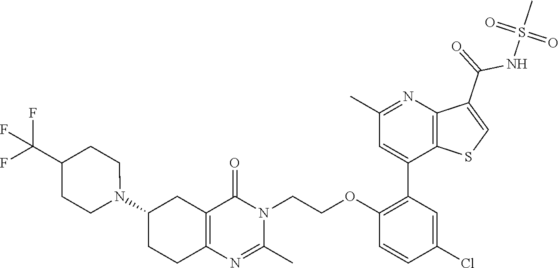 [SMILES: Cc1cc(-c2cc(Cl)ccc2OCCn2c(C)nc3c(c2=O)C[C@@H](N2CCC(C(F)(F)F)CC2)CC3)c2scc(C(=O)NS(C)(=O)=O)c2n1] |
| 434 | 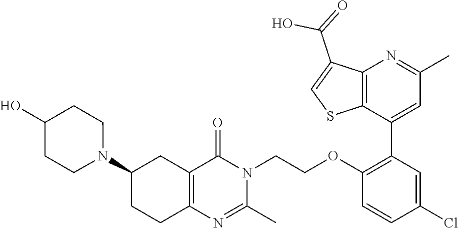 [SMILES: Cc1cc(-c2cc(Cl)ccc2OCCn2c(C)nc3c(c2=O)C[C@H](N2CCC(O)CC2)CC3)c2scc(C(=O)O)c2n1] |
| 435 | 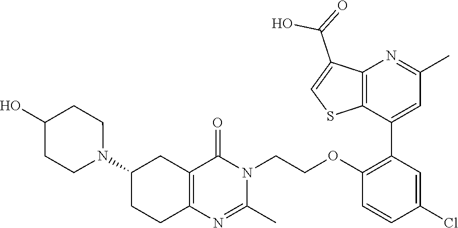 [SMILES: Cc1cc(-c2cc(Cl)ccc2OCCn2c(C)nc3c(c2=O)C[C@@H](N2CCC(O)CC2)CC3)c2scc(C(=O)O)c2n1] |
| 436 | 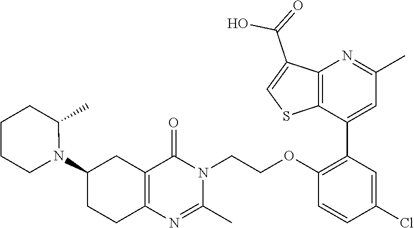 [SMILES: Cc1cc(-c2cc(Cl)ccc2OCCn2c(C)nc3c(c2=O)C[C@H](N2CCCC[C@@H]2C)CC3)c2scc(C(=O)O)c2n1] |
| 437 | 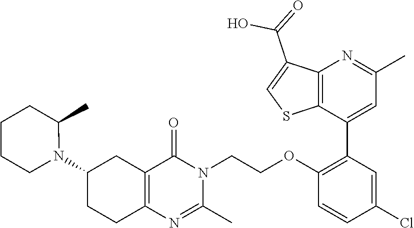 [SMILES: Cc1cc(-c2cc(Cl)ccc2OCCn2c(C)nc3c(c2=O)C[C@@H](N2CCCC[C@H]2C)CC3)c2scc(C(=O)O)c2n1] |
| 438 | 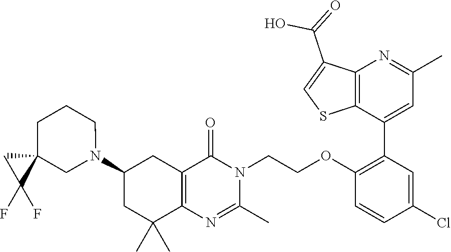 [SMILES: Cc1cc(-c2cc(Cl)ccc2OCCn2c(C)nc3c(c2=O)C[C@H](N2CCC[C@]4(C2)CC4(F)F)CC3(C)C)c2scc(C(=O)O)c2n1] |
| 439 | 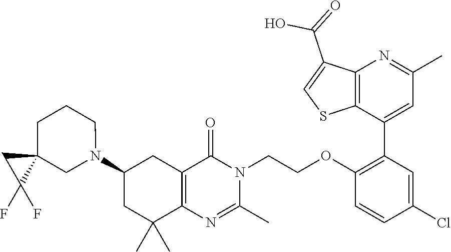 [SMILES: Cc1cc(-c2cc(Cl)ccc2OCCn2c(C)nc3c(c2=O)C[C@H](N2CCC[C@@]4(C2)CC4(F)F)CC3(C)C)c2scc(C(=O)O)c2n1] |
| 440 | 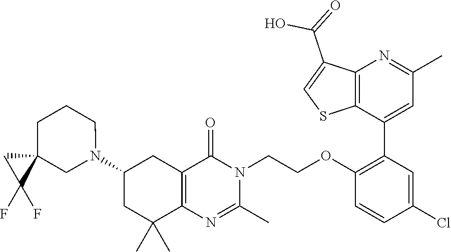 [SMILES: Cc1cc(-c2cc(Cl)ccc2OCCn2c(C)nc3c(c2=O)C[C@@H](N2CCC[C@]4(C2)CC4(F)F)CC3(C)C)c2scc(C(=O)O)c2n1] |
| 441 | 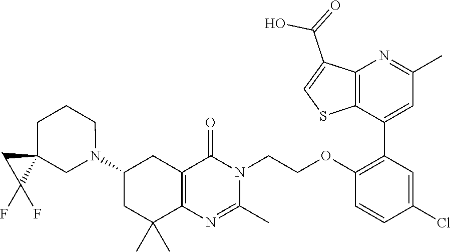 [SMILES: Cc1cc(-c2cc(Cl)ccc2OCCn2c(C)nc3c(c2=O)C[C@@H](N2CCC[C@@]4(C2)CC4(F)F)CC3(C)C)c2scc(C(=O)O)c2n1] |
| 442 | 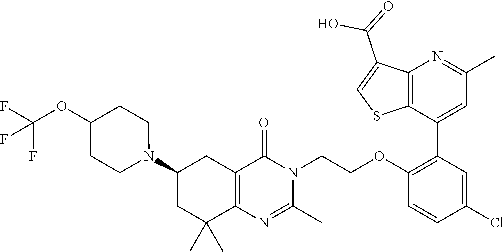 [SMILES: Cc1cc(-c2cc(Cl)ccc2OCCn2c(C)nc3c(c2=O)C[C@H](N2CCC(OC(F)(F)F)CC2)CC3(C)C)c2scc(C(=O)O)c2n1] |
| 443 | 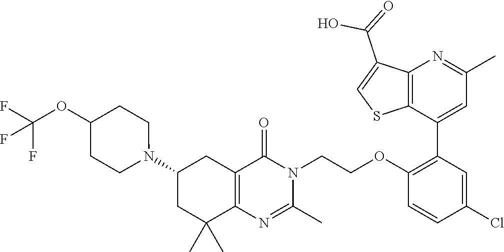 [SMILES: Cc1cc(-c2cc(Cl)ccc2OCCn2c(C)nc3c(c2=O)C[C@@H](N2CCC(OC(F)(F)F)CC2)CC3(C)C)c2scc(C(=O)O)c2n1] |
| 444 | 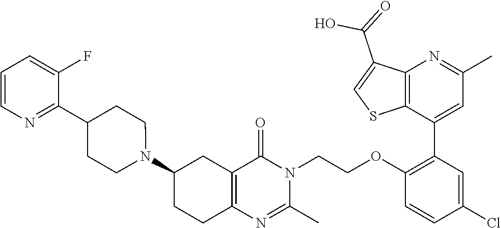 [SMILES: Cc1cc(-c2cc(Cl)ccc2OCCn2c(C)nc3c(c2=O)C[C@H](N2CCC(c4ncccc4F)CC2)CC3)c2scc(C(=O)O)c2n1] |
| 445 | 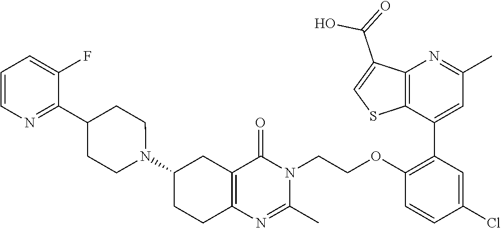 [SMILES: Cc1cc(-c2cc(Cl)ccc2OCCn2c(C)nc3c(c2=O)C[C@@H](N2CCC(c4ncccc4F)CC2)CC3)c2scc(C(=O)O)c2n1] |
| 446 | 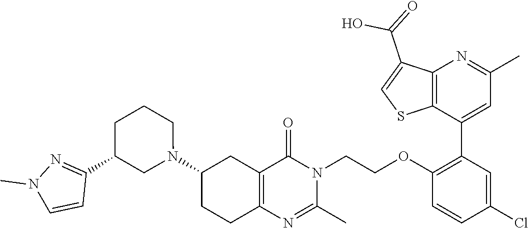 |
| 447 | 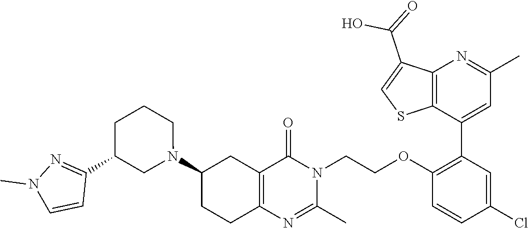 |
| 448 | 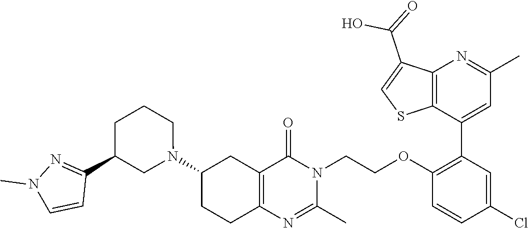 |
| 449 | 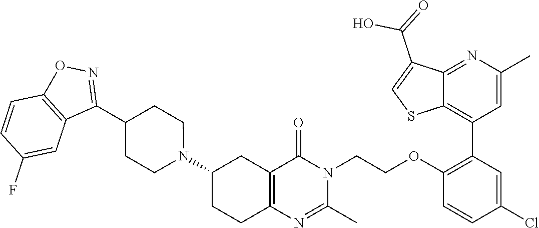 [SMILES: Cc1cc(-c2cc(Cl)ccc2OCCn2c(C)nc3c(c2=O)C[C@@H](N2CCC(c4noc5ccc(F)cc45)CC2)CC3)c2scc(C(=O)O)c2n1] |
| 450 | 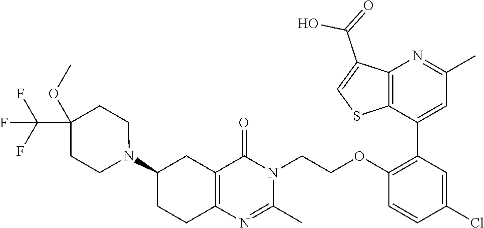 [SMILES: COC1(C(F)(F)F)CCN([C@@H]2CCc3nc(C)n(CCOc4ccc(Cl)cc4-c4cc(C)nc5c(C(=O)O)csc45)c(=O)c3C2)CC1] |
| 451 | 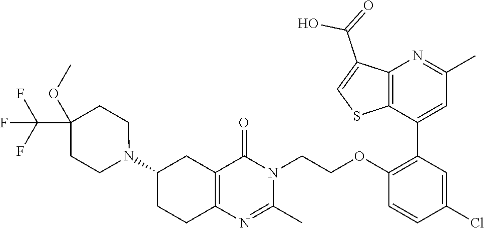 [SMILES: COC1(C(F)(F)F)CCN([C@H]2CCc3nc(C)n(CCOc4ccc(Cl)cc4-c4cc(C)nc5c(C(=O)O)csc45)c(=O)c3C2)CC1] |
| 452 | 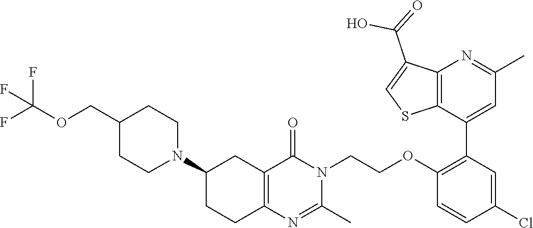 [SMILES: Cc1cc(-c2cc(Cl)ccc2OCCn2c(C)nc3c(c2=O)C[C@H](N2CCC(COC(F)(F)F)CC2)CC3)c2scc(C(=O)O)c2n1] |
| 453 | 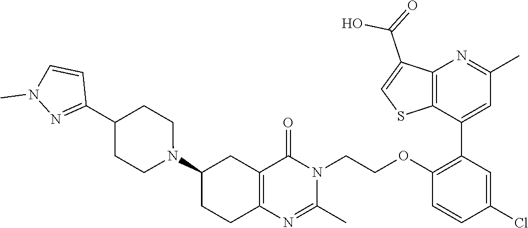 |
| 454 | 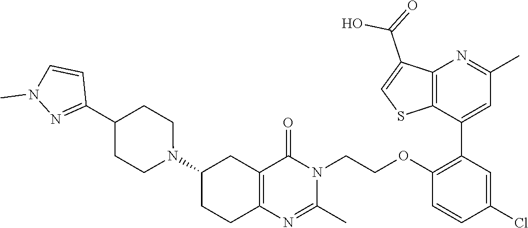 |
| 455 | 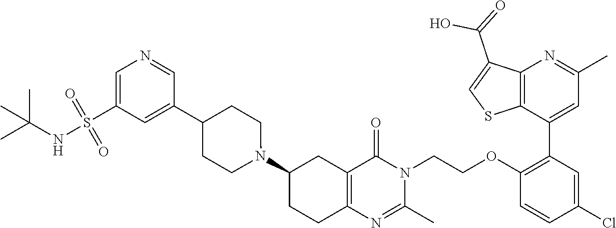 [SMILES: Cc1cc(-c2cc(Cl)ccc2OCCn2c(C)nc3c(c2=O)C[C@H](N2CCC(c4cncc(S(=O)(=O)NC(C)(C)C)c4)CC2)CC3)c2scc(C(=O)O)c2n1] |
| 456 | 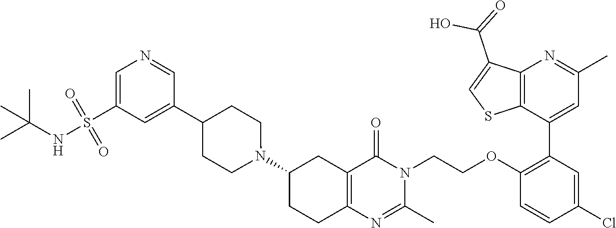 [SMILES: Cc1cc(-c2cc(Cl)ccc2OCCn2c(C)nc3c(c2=O)C[C@@H](N2CCC(c4cncc(S(=O)(=O)NC(C)(C)C)c4)CC2)CC3)c2scc(C(=O)O)c2n1] |
| 457 | 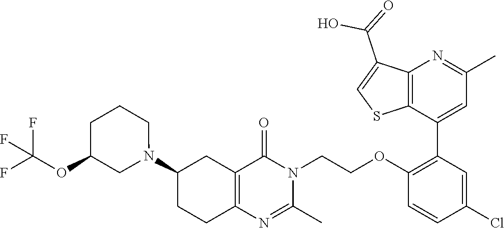 [SMILES: Cc1cc(-c2cc(Cl)ccc2OCCn2c(C)nc3c(c2=O)C[C@H](N2CCC[C@H](OC(F)(F)F)C2)CC3)c2scc(C(=O)O)c2n1] |
| 458 | 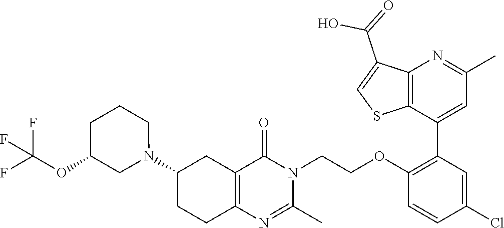 [SMILES: Cc1cc(-c2cc(Cl)ccc2OCCn2c(C)nc3c(c2=O)C[C@@H](N2CCC[C@@H](OC(F)(F)F)C2)CC3)c2scc(C(=O)O)c2n1] |
| 459 | 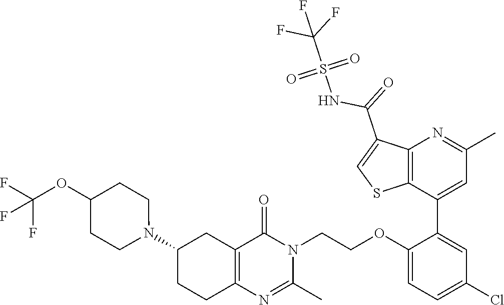 [SMILES: Cc1cc(-c2cc(Cl)ccc2OCCn2c(C)nc3c(c2=O)C[C@@H](N2CCC(OC(F)(F)F)CC2)CC3)c2scc(C(=O)NS(=O)(=O)C(F)(F)F)c2n1] |
| 460 | 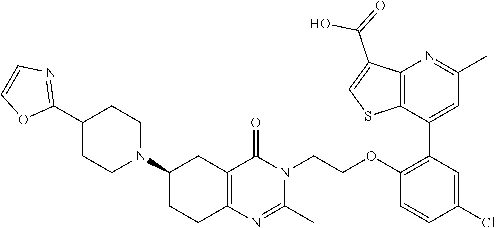 [SMILES: Cc1cc(-c2cc(Cl)ccc2OCCn2c(C)nc3c(c2=O)C[C@H](N2CCC(c4ncco4)CC2)CC3)c2scc(C(=O)O)c2n1] |
| 461 | 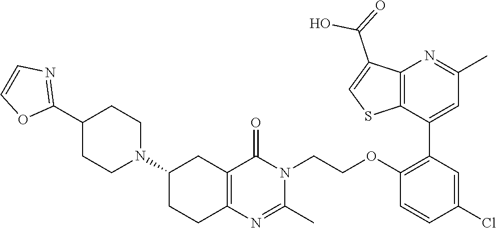 [SMILES: Cc1cc(-c2cc(Cl)ccc2OCCn2c(C)nc3c(c2=O)C[C@@H](N2CCC(c4ncco4)CC2)CC3)c2scc(C(=O)O)c2n1] |
| 462 | 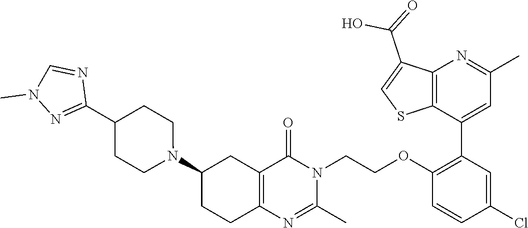 |
| 463 | 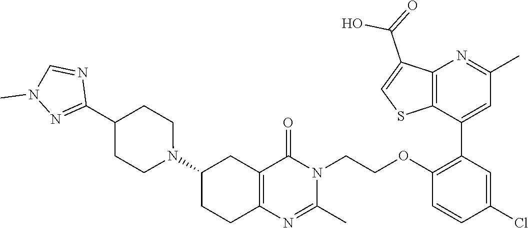 |
| 464 | 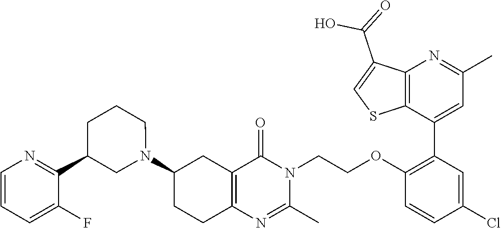 [SMILES: Cc1cc(-c2cc(Cl)ccc2OCCn2c(C)nc3c(c2=O)C[C@H](N2CCC[C@H](c4ncccc4F)C2)CC3)c2scc(C(=O)O)c2n1] |
| 465 | 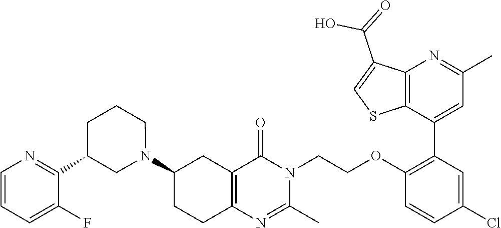 [SMILES: Cc1cc(-c2cc(Cl)ccc2OCCn2c(C)nc3c(c2=O)C[C@H](N2CCC[C@@H](c4ncccc4F)C2)CC3)c2scc(C(=O)O)c2n1] |
| 466 | 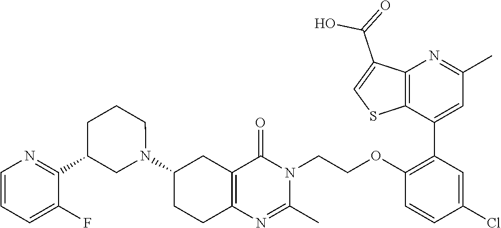 [SMILES: Cc1cc(-c2cc(Cl)ccc2OCCn2c(C)nc3c(c2=O)C[C@@H](N2CCC[C@@H](c4ncccc4F)C2)CC3)c2scc(C(=O)O)c2n1] |
| 467 | 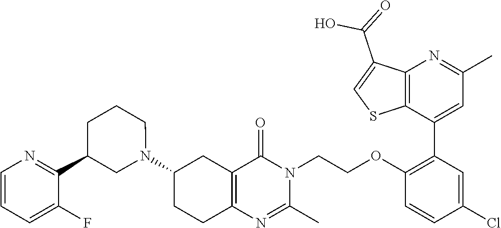 [SMILES: Cc1cc(-c2cc(Cl)ccc2OCCn2c(C)nc3c(c2=O)C[C@@H](N2CCC[C@H](c4ncccc4F)C2)CC3)c2scc(C(=O)O)c2n1] |
| 468 | 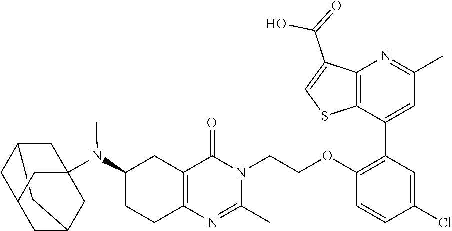 [SMILES: Cc1cc(-c2cc(Cl)ccc2OCCn2c(C)nc3c(c2=O)C[C@H](N(C)C24CC5CC(CC(C5)C2)C4)CC3)c2scc(C(=O)O)c2n1] |
| 469 | 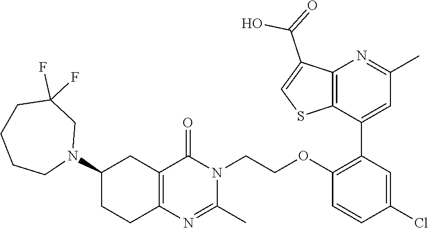 [SMILES: Cc1cc(-c2cc(Cl)ccc2OCCn2c(C)nc3c(c2=O)C[C@H](N2CCCCC(F)(F)C2)CC3)c2scc(C(=O)O)c2n1] |
| 470 | 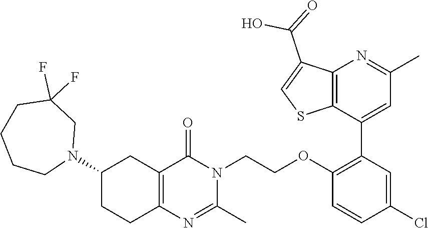 [SMILES: Cc1cc(-c2cc(Cl)ccc2OCCn2c(C)nc3c(c2=O)C[C@@H](N2CCCCC(F)(F)C2)CC3)c2scc(C(=O)O)c2n1] |
| 471 | 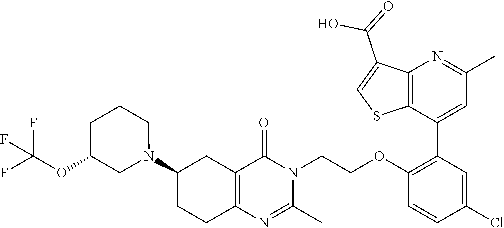 [SMILES: Cc1cc(-c2cc(Cl)ccc2OCCn2c(C)nc3c(c2=O)C[C@H](N2CCC[C@@H](OC(F)(F)F)C2)CC3)c2scc(C(=O)O)c2n1] |
| 472 | 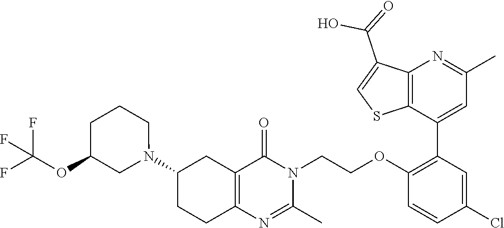 [SMILES: Cc1cc(-c2cc(Cl)ccc2OCCn2c(C)nc3c(c2=O)C[C@@H](N2CCC[C@H](OC(F)(F)F)C2)CC3)c2scc(C(=O)O)c2n1] |
| 473 | 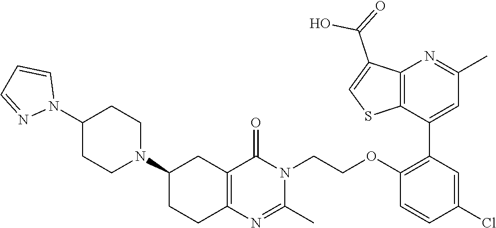 [SMILES: Cc1cc(-c2cc(Cl)ccc2OCCn2c(C)nc3c(c2=O)C[C@H](N2CCC(n4cccn4)CC2)CC3)c2scc(C(=O)O)c2n1] |
| 474 | 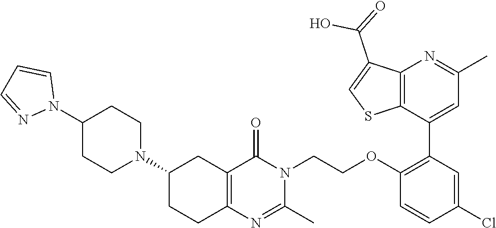 [SMILES: Cc1cc(-c2cc(Cl)ccc2OCCn2c(C)nc3c(c2=O)C[C@@H](N2CCC(n4cccn4)CC2)CC3)c2scc(C(=O)O)c2n1] |
| 475 | 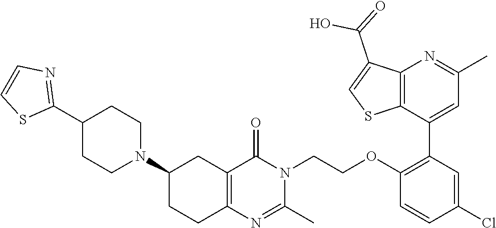 [SMILES: Cc1cc(-c2cc(Cl)ccc2OCCn2c(C)nc3c(c2=O)C[C@H](N2CCC(c4nccs4)CC2)CC3)c2scc(C(=O)O)c2n1] |
| 476 | 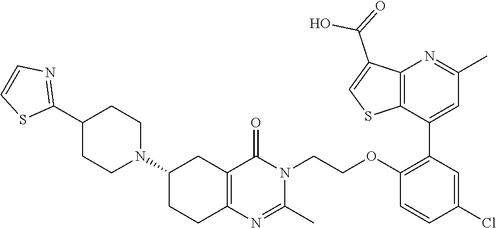 [SMILES: Cc1cc(-c2cc(Cl)ccc2OCCn2c(C)nc3c(c2=O)C[C@@H](N2CCC(c4nccs4)CC2)CC3)c2scc(C(=O)O)c2n1] |
| 477 | 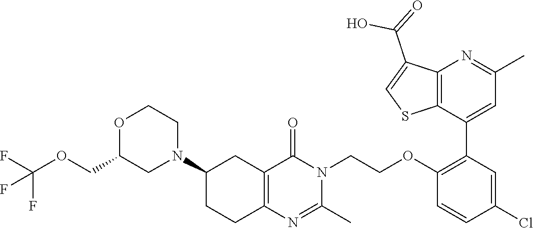 [SMILES: Cc1cc(-c2cc(Cl)ccc2OCCn2c(C)nc3c(c2=O)C[C@H](N2CCO[C@@H](COC(F)(F)F)C2)CC3)c2scc(C(=O)O)c2n1] |
| 478 | 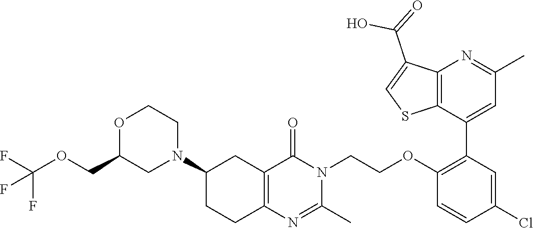 [SMILES: Cc1cc(-c2cc(Cl)ccc2OCCn2c(C)nc3c(c2=O)C[C@H](N2CCO[C@H](COC(F)(F)F)C2)CC3)c2scc(C(=O)O)c2n1] |
| 479 | 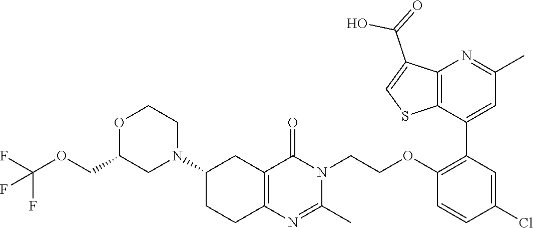 [SMILES: Cc1cc(-c2cc(Cl)ccc2OCCn2c(C)nc3c(c2=O)C[C@@H](N2CCO[C@@H](COC(F)(F)F)C2)CC3)c2scc(C(=O)O)c2n1] |
| 480 | 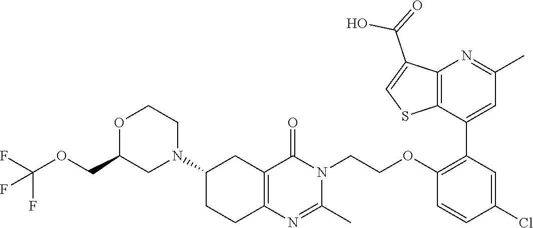 [SMILES: Cc1cc(-c2cc(Cl)ccc2OCCn2c(C)nc3c(c2=O)C[C@@H](N2CCO[C@H](COC(F)(F)F)C2)CC3)c2scc(C(=O)O)c2n1] |
| 481 | 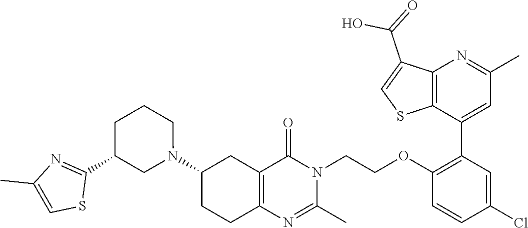 |
| 482 | 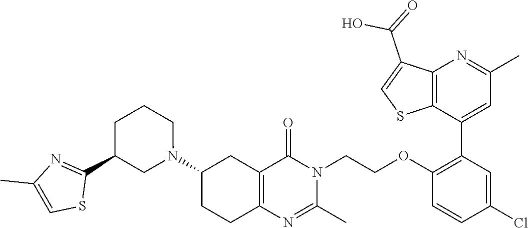 |
| 483 | 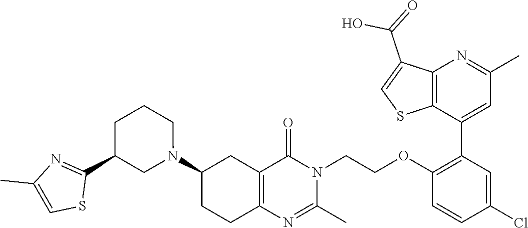 |
| 484 | 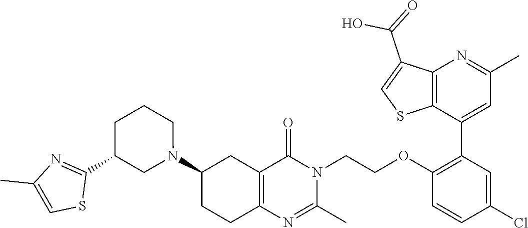 |
| 485 | 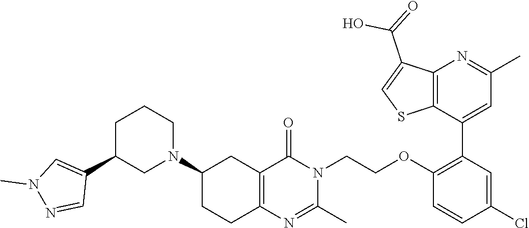 |
| 486 | 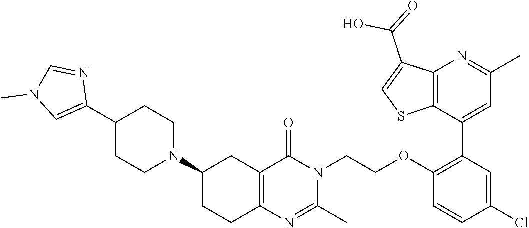 |
| 487 | 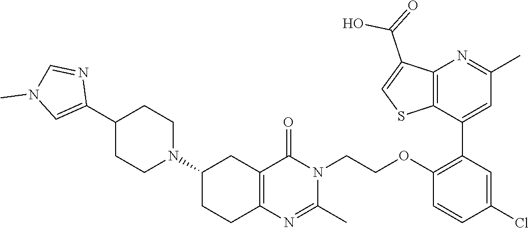 |
| 488 | 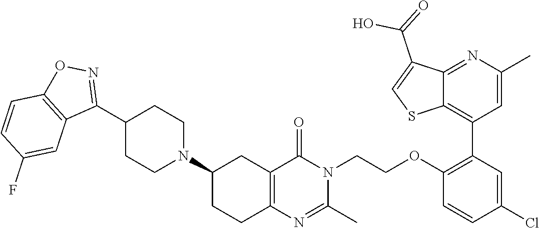 [SMILES: Cc1cc(-c2cc(Cl)ccc2OCCn2c(C)nc3c(c2=O)C[C@H](N2CCC(c4noc5ccc(F)cc45)CC2)CC3)c2scc(C(=O)O)c2n1] |
| 489 | 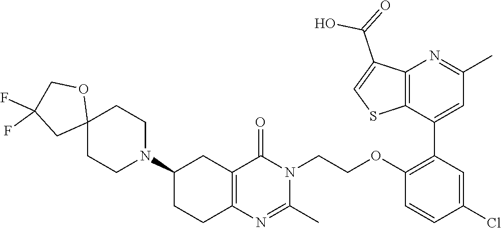 [SMILES: Cc1cc(-c2cc(Cl)ccc2OCCn2c(C)nc3c(c2=O)C[C@H](N2CCC4(CC2)CC(F)(F)CO4)CC3)c2scc(C(=O)O)c2n1] |
| 490 | 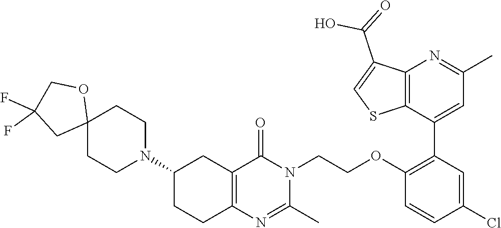 [SMILES: Cc1cc(-c2cc(Cl)ccc2OCCn2c(C)nc3c(c2=O)C[C@@H](N2CCC4(CC2)CC(F)(F)CO4)CC3)c2scc(C(=O)O)c2n1] |
| 491 | 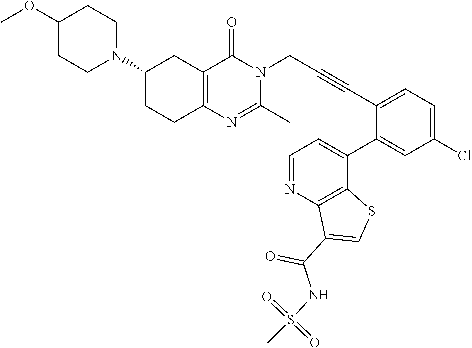 [SMILES: COC1CCN([C@H]2CCc3nc(C)n(CC#Cc4ccc(Cl)cc4-c4ccnc5c(C(=O)NS(C)(=O)=O)csc45)c(=O)c3C2)CC1] |
| 492 | 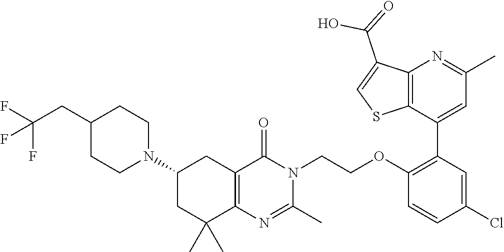 [SMILES: Cc1cc(-c2cc(Cl)ccc2OCCn2c(C)nc3c(c2=O)C[C@@H](N2CCC(CC(F)(F)F)CC2)CC3(C)C)c2scc(C(=O)O)c2n1] |
| 493 | 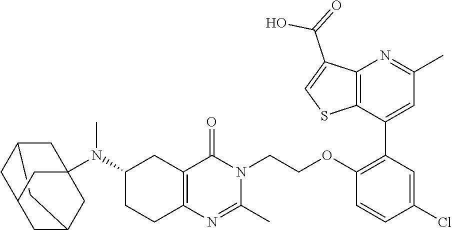 [SMILES: Cc1cc(-c2cc(Cl)ccc2OCCn2c(C)nc3c(c2=O)C[C@@H](N(C)C24CC5CC(CC(C5)C2)C4)CC3)c2scc(C(=O)O)c2n1] |
| 494 | 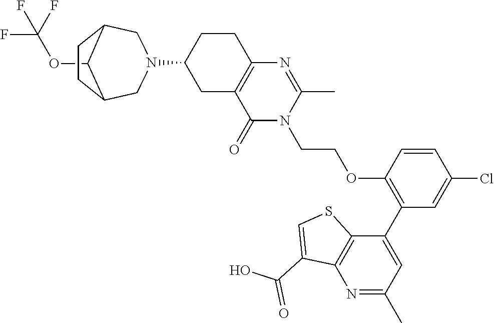 [SMILES: Cc1cc(-c2cc(Cl)ccc2OCCn2c(C)nc3c(c2=O)C[C@H](N2CC4CCC(C2)C4OC(F)(F)F)CC3)c2scc(C(=O)O)c2n1] |
| 495 | 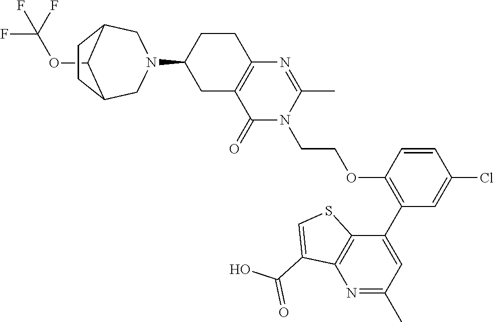 [SMILES: Cc1cc(-c2cc(Cl)ccc2OCCn2c(C)nc3c(c2=O)C[C@@H](N2CC4CCC(C2)C4OC(F)(F)F)CC3)c2scc(C(=O)O)c2n1] |
| 496 | 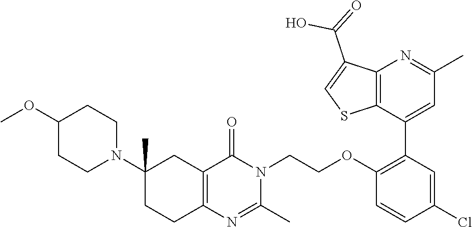 |
| 497 | 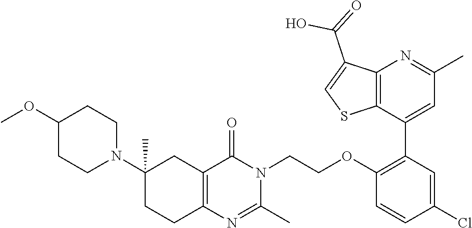 |
| 498 | 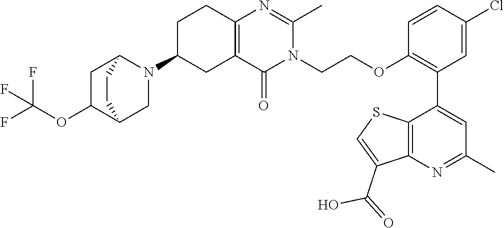 [SMILES: Cc1cc(-c2cc(Cl)ccc2OCCn2c(C)nc3c(c2=O)C[C@@H](N2C[C@@H]4CC[C@H]2CC4OC(F)(F)F)CC3)c2scc(C(=O)O)c2n1] |
| 499 | 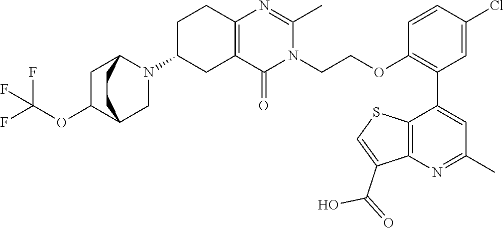 [SMILES: Cc1cc(-c2cc(Cl)ccc2OCCn2c(C)nc3c(c2=O)C[C@H](N2C[C@H]4CC[C@@H]2CC4OC(F)(F)F)CC3)c2scc(C(=O)O)c2n1] |
| 500 | 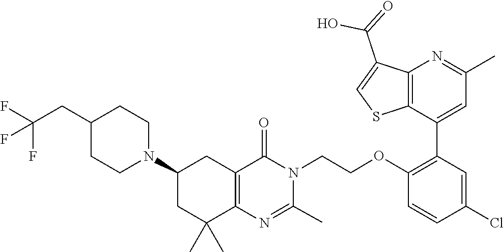 [SMILES: Cc1cc(-c2cc(Cl)ccc2OCCn2c(C)nc3c(c2=O)C[C@H](N2CCC(CC(F)(F)F)CC2)CC3(C)C)c2scc(C(=O)O)c2n1] |
| 501 | 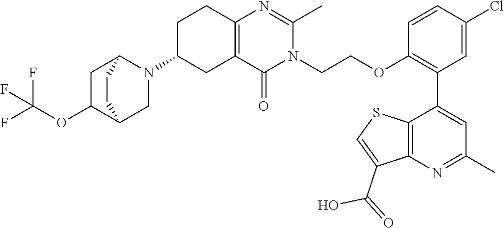 [SMILES: Cc1cc(-c2cc(Cl)ccc2OCCn2c(C)nc3c(c2=O)C[C@H](N2C[C@@H]4CC[C@H]2CC4OC(F)(F)F)CC3)c2scc(C(=O)O)c2n1] |
| 502 | 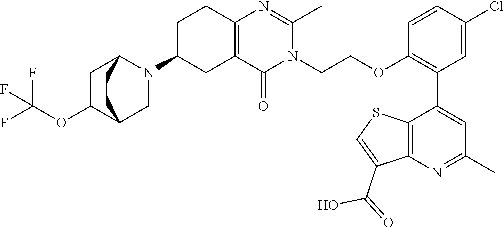 [SMILES: Cc1cc(-c2cc(Cl)ccc2OCCn2c(C)nc3c(c2=O)C[C@@H](N2C[C@H]4CC[C@@H]2CC4OC(F)(F)F)CC3)c2scc(C(=O)O)c2n1] |
| 503 | 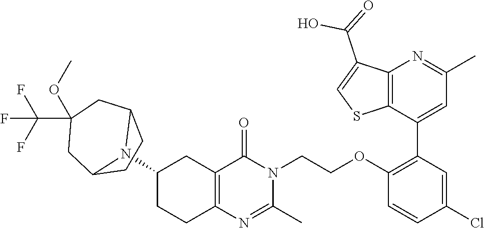 [SMILES: COC1(C(F)(F)F)CC2CCC(C1)N2[C@H]1CCc2nc(C)n(CCOc3ccc(Cl)cc3-c3cc(C)nc4c(C(=O)O)csc34)c(=O)c2C1] |
| 504 | 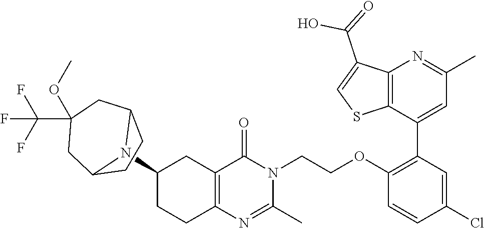 [SMILES: COC1(C(F)(F)F)CC2CCC(C1)N2[C@@H]1CCc2nc(C)n(CCOc3ccc(Cl)cc3-c3cc(C)nc4c(C(=O)O)csc34)c(=O)c2C1] |
| 505 | 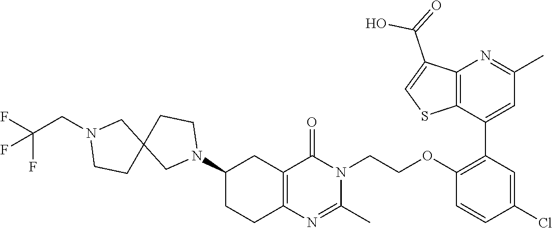 [SMILES: Cc1cc(-c2cc(Cl)ccc2OCCn2c(C)nc3c(c2=O)C[C@H](N2CCC4(CCN(CC(F)(F)F)C4)C2)CC3)c2scc(C(=O)O)c2n1] |
| 506 | 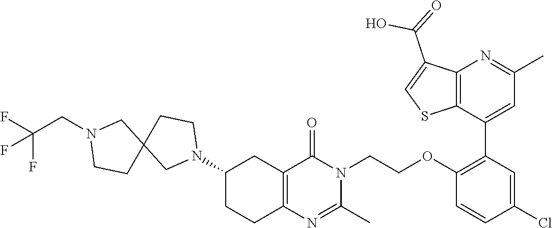 [SMILES: Cc1cc(-c2cc(Cl)ccc2OCCn2c(C)nc3c(c2=O)C[C@@H](N2CCC4(CCN(CC(F)(F)F)C4)C2)CC3)c2scc(C(=O)O)c2n1] |
| 507 | 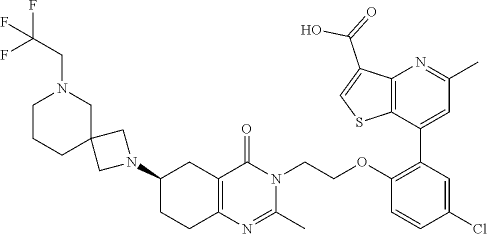 [SMILES: Cc1cc(-c2cc(Cl)ccc2OCCn2c(C)nc3c(c2=O)C[C@H](N2CC4(CCCN(CC(F)(F)F)C4)C2)CC3)c2scc(C(=O)O)c2n1] |
| 508 | 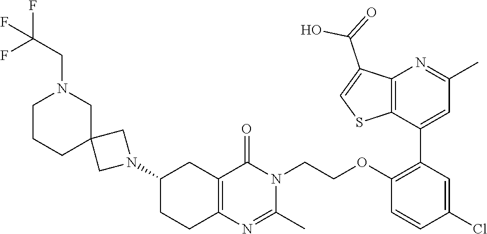 [SMILES: Cc1cc(-c2cc(Cl)ccc2OCCn2c(C)nc3c(c2=O)C[C@@H](N2CC4(CCCN(CC(F)(F)F)C4)C2)CC3)c2scc(C(=O)O)c2n1] |
| 509 | 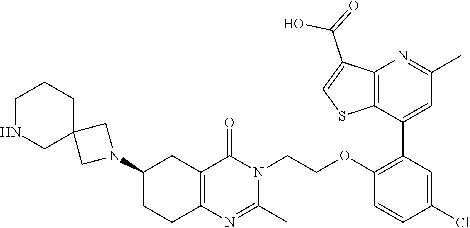 [SMILES: Cc1cc(-c2cc(Cl)ccc2OCCn2c(C)nc3c(c2=O)C[C@H](N2CC4(CCCNC4)C2)CC3)c2scc(C(=O)O)c2n1] |
| 510 | 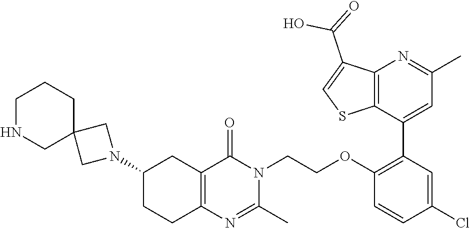 [SMILES: Cc1cc(-c2cc(Cl)ccc2OCCn2c(C)nc3c(c2=O)C[C@@H](N2CC4(CCCNC4)C2)CC3)c2scc(C(=O)O)c2n1] |
| 511 | 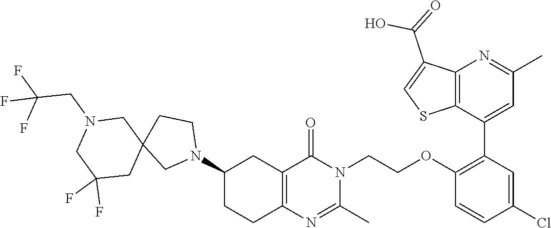 [SMILES: Cc1cc(-c2cc(Cl)ccc2OCCn2c(C)nc3c(c2=O)C[C@H](N2CCC4(CN(CC(F)(F)F)CC(F)(F)C4)C2)CC3)c2scc(C(=O)O)c2n1] |
| 512 | 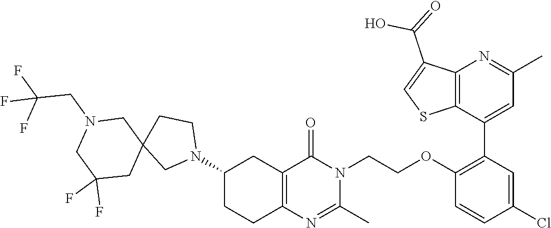 [SMILES: Cc1cc(-c2cc(Cl)ccc2OCCn2c(C)nc3c(c2=O)C[C@@H](N2CCC4(CN(CC(F)(F)F)CC(F)(F)C4)C2)CC3)c2scc(C(=O)O)c2n1] |
| 513 | 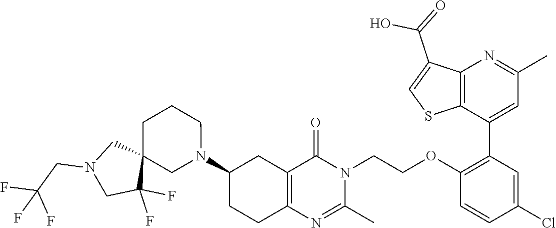 [SMILES: Cc1cc(-c2cc(Cl)ccc2OCCn2c(C)nc3c(c2=O)C[C@H](N2CCC[C@@]4(CN(CC(F)(F)F)CC4(F)F)C2)CC3)c2scc(C(=O)O)c2n1] |
| 514 | 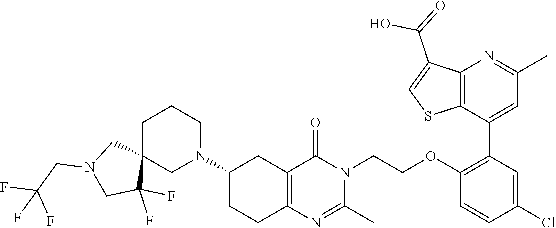 [SMILES: Cc1cc(-c2cc(Cl)ccc2OCCn2c(C)nc3c(c2=O)C[C@@H](N2CCC[C@@]4(CN(CC(F)(F)F)CC4(F)F)C2)CC3)c2scc(C(=O)O)c2n1] |
| 515 | 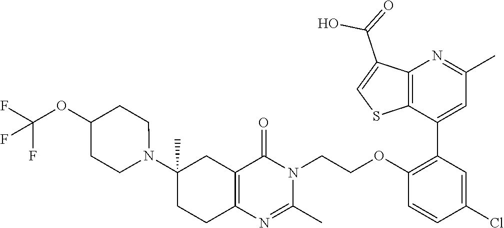 [SMILES: Cc1cc(-c2cc(Cl)ccc2OCCn2c(C)nc3c(c2=O)C[C@](C)(N2CCC(OC(F)(F)F)CC2)CC3)c2scc(C(=O)O)c2n1] |
| 516 | 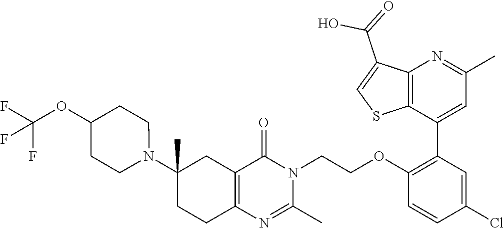 [SMILES: Cc1cc(-c2cc(Cl)ccc2OCCn2c(C)nc3c(c2=O)C[C@@](C)(N2CCC(OC(F)(F)F)CC2)CC3)c2scc(C(=O)O)c2n1] |
| 517 | 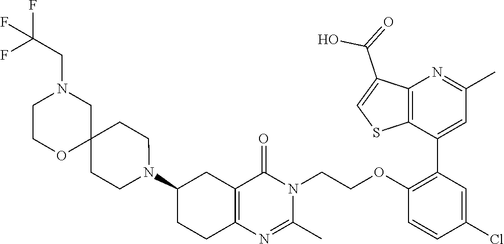 [SMILES: Cc1cc(-c2cc(Cl)ccc2OCCn2c(C)nc3c(c2=O)C[C@H](N2CCC4(CC2)CN(CC(F)(F)F)CCO4)CC3)c2scc(C(=O)O)c2n1] |
| 518 | 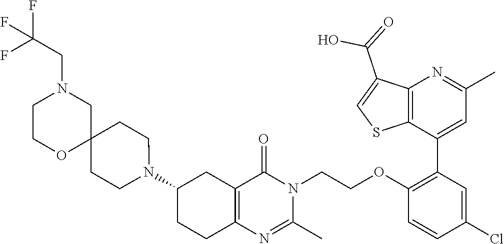 [SMILES: Cc1cc(-c2cc(Cl)ccc2OCCn2c(C)nc3c(c2=O)C[C@@H](N2CCC4(CC2)CN(CC(F)(F)F)CCO4)CC3)c2scc(C(=O)O)c2n1] |
| 519 | 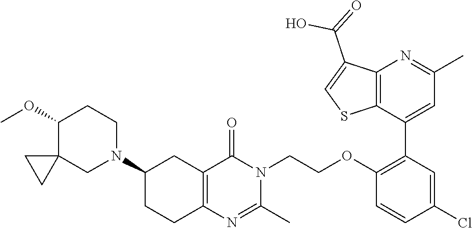 |
| 520 | 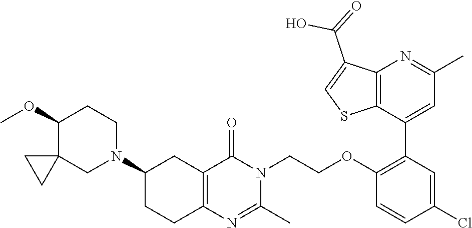 |
| 521 | 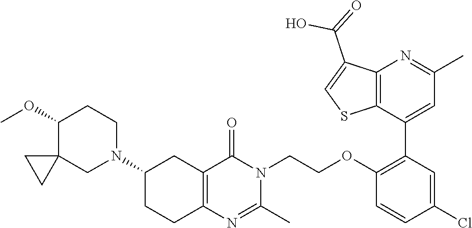 |
| 522 | 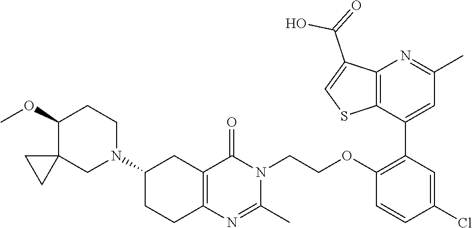 |
| 523 | 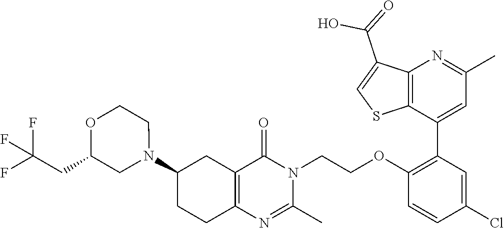 [SMILES: Cc1cc(-c2cc(Cl)ccc2OCCn2c(C)nc3c(c2=O)C[C@H](N2CCO[C@@H](CC(F)(F)F)C2)CC3)c2scc(C(=O)O)c2n1] |
| 524 | 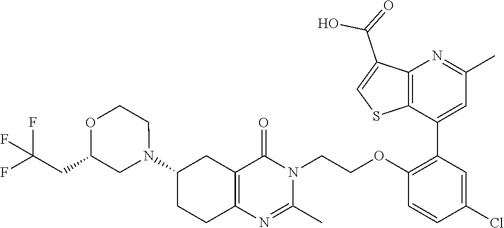 [SMILES: Cc1cc(-c2cc(Cl)ccc2OCCn2c(C)nc3c(c2=O)C[C@@H](N2CCO[C@@H](CC(F)(F)F)C2)CC3)c2scc(C(=O)O)c2n1] |
| 525 | 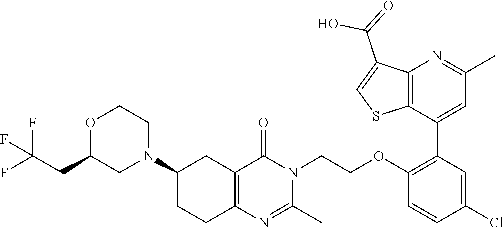 [SMILES: Cc1cc(-c2cc(Cl)ccc2OCCn2c(C)nc3c(c2=O)C[C@H](N2CCO[C@H](CC(F)(F)F)C2)CC3)c2scc(C(=O)O)c2n1] |
| 526 | 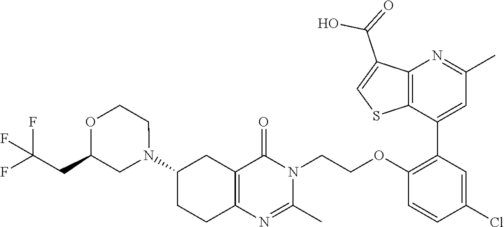 [SMILES: Cc1cc(-c2cc(Cl)ccc2OCCn2c(C)nc3c(c2=O)C[C@@H](N2CCO[C@H](CC(F)(F)F)C2)CC3)c2scc(C(=O)O)c2n1] |
| 527 | 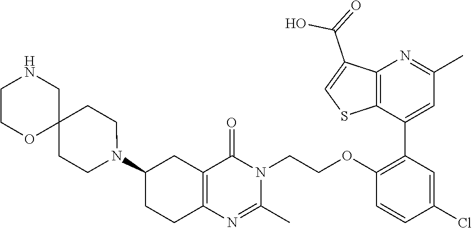 [SMILES: Cc1cc(-c2cc(Cl)ccc2OCCn2c(C)nc3c(c2=O)C[C@H](N2CCC4(CC2)CNCCO4)CC3)c2scc(C(=O)O)c2n1] |
| 528 | 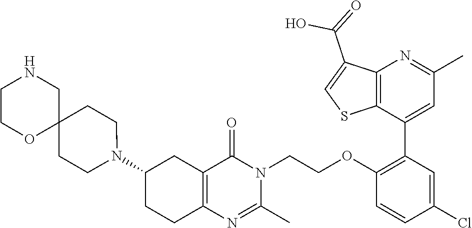 [SMILES: Cc1cc(-c2cc(Cl)ccc2OCCn2c(C)nc3c(c2=O)C[C@@H](N2CCC4(CC2)CNCCO4)CC3)c2scc(C(=O)O)c2n1] |
| 529 | 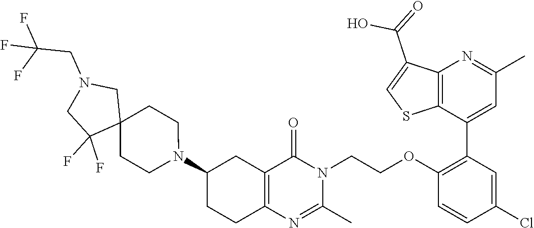 [SMILES: Cc1cc(-c2cc(Cl)ccc2OCCn2c(C)nc3c(c2=O)C[C@H](N2CCC4(CC2)CN(CC(F)(F)F)CC4(F)F)CC3)c2scc(C(=O)O)c2n1] |
| 530 | 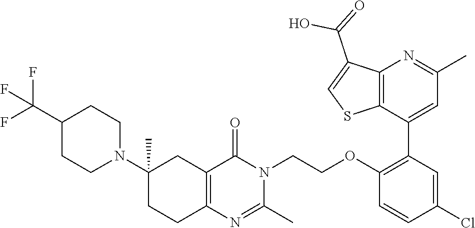 [SMILES: Cc1cc(-c2cc(Cl)ccc2OCCn2c(C)nc3c(c2=O)C[C@](C)(N2CCC(C(F)(F)F)CC2)CC3)c2scc(C(=O)O)c2n1] |
| 531 | 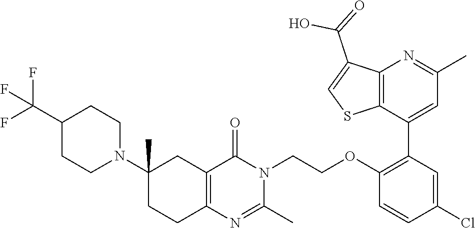 [SMILES: Cc1cc(-c2cc(Cl)ccc2OCCn2c(C)nc3c(c2=O)C[C@@](C)(N2CCC(C(F)(F)F)CC2)CC3)c2scc(C(=O)O)c2n1] |
| 532 | 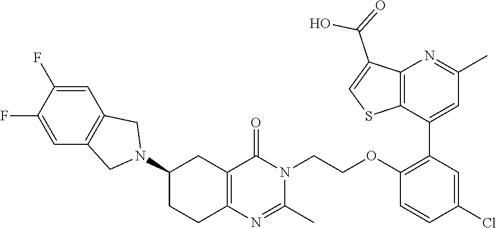 [SMILES: Cc1cc(-c2cc(Cl)ccc2OCCn2c(C)nc3c(c2=O)C[C@H](N2Cc4cc(F)c(F)cc4C2)CC3)c2scc(C(=O)O)c2n1] |
| 533 | 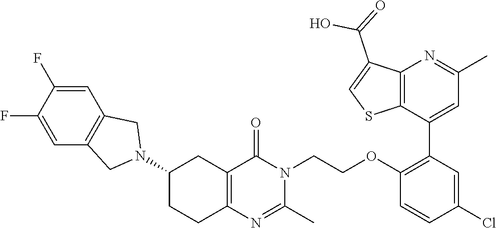 [SMILES: Cc1cc(-c2cc(Cl)ccc2OCCn2c(C)nc3c(c2=O)C[C@@H](N2Cc4cc(F)c(F)cc4C2)CC3)c2scc(C(=O)O)c2n1] |
| 534 | 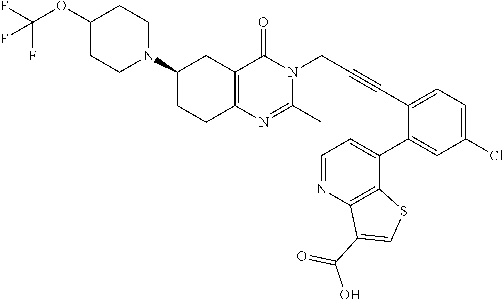 [SMILES: Cc1nc2c(c(=O)n1CC#Cc1ccc(Cl)cc1-c1ccnc3c(C(=O)O)csc13)C[C@H](N1CCC(OC(F)(F)F)CC1)CC2] |
| 535 | 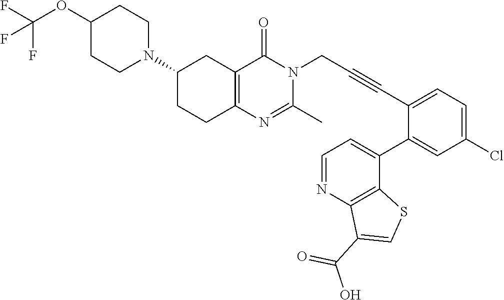 [SMILES: Cc1nc2c(c(=O)n1CC#Cc1ccc(Cl)cc1-c1ccnc3c(C(=O)O)csc13)C[C@@H](N1CCC(OC(F)(F)F)CC1)CC2] |
| 536 | 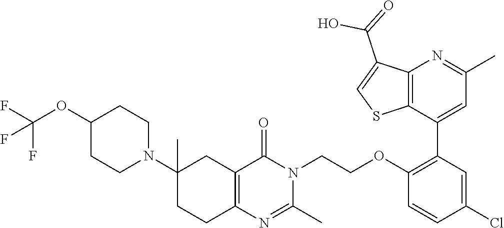 [SMILES: Cc1cc(-c2cc(Cl)ccc2OCCn2c(C)nc3c(c2=O)CC(C)(N2CCC(OC(F)(F)F)CC2)CC3)c2scc(C(=O)O)c2n1] |
| 537 | 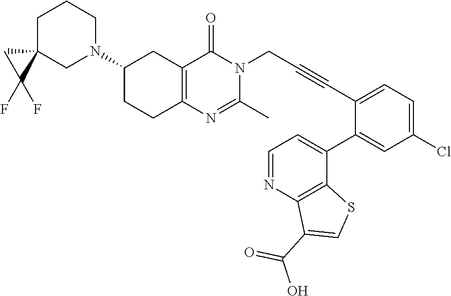 [SMILES: Cc1nc2c(c(=O)n1CC#Cc1ccc(Cl)cc1-c1ccnc3c(C(=O)O)csc13)C[C@@H](N1CCC[C@]3(C1)CC3(F)F)CC2] |
| 538 | 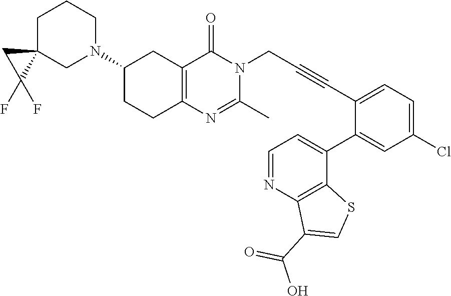 [SMILES: Cc1nc2c(c(=O)n1CC#Cc1ccc(Cl)cc1-c1ccnc3c(C(=O)O)csc13)C[C@@H](N1CCC[C@@]3(C1)CC3(F)F)CC2] |
| 539 | 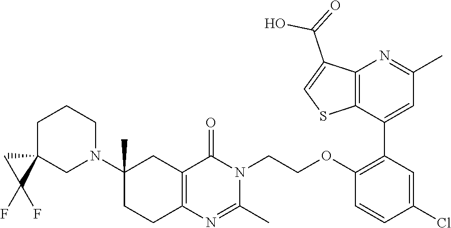 [SMILES: Cc1cc(-c2cc(Cl)ccc2OCCn2c(C)nc3c(c2=O)C[C@@](C)(N2CCC[C@]4(C2)CC4(F)F)CC3)c2scc(C(=O)O)c2n1] |
| 540 | 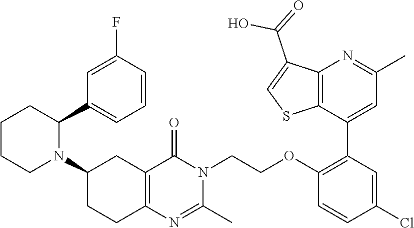 [SMILES: Cc1cc(-c2cc(Cl)ccc2OCCn2c(C)nc3c(c2=O)C[C@H](N2CCCC[C@H]2c2cccc(F)c2)CC3)c2scc(C(=O)O)c2n1] |
| 541 | 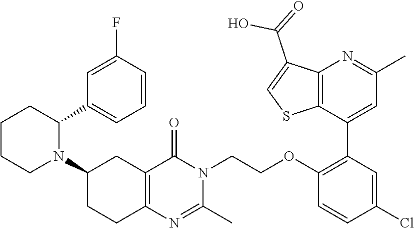 [SMILES: Cc1cc(-c2cc(Cl)ccc2OCCn2c(C)nc3c(c2=O)C[C@H](N2CCCC[C@@H]2c2cccc(F)c2)CC3)c2scc(C(=O)O)c2n1] |
| 542 | 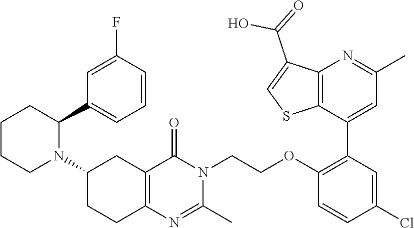 [SMILES: Cc1cc(-c2cc(Cl)ccc2OCCn2c(C)nc3c(c2=O)C[C@@H](N2CCCC[C@H]2c2cccc(F)c2)CC3)c2scc(C(=O)O)c2n1] |
| 543 | 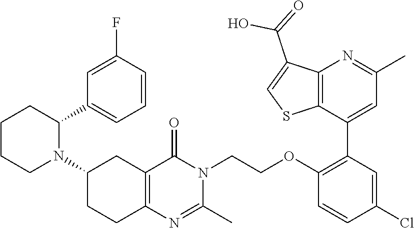 [SMILES: Cc1cc(-c2cc(Cl)ccc2OCCn2c(C)nc3c(c2=O)C[C@@H](N2CCCC[C@@H]2c2cccc(F)c2)CC3)c2scc(C(=O)O)c2n1] |
| 544 | 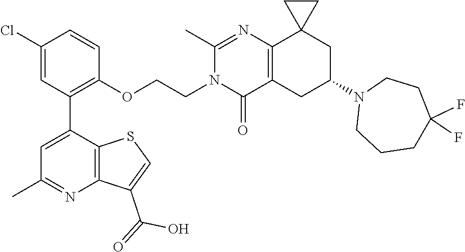 [SMILES: Cc1cc(-c2cc(Cl)ccc2OCCn2c(C)nc3c(c2=O)C[C@@H](N2CCCC(F)(F)CC2)CC32CC2)c2scc(C(=O)O)c2n1] |
| 545 | 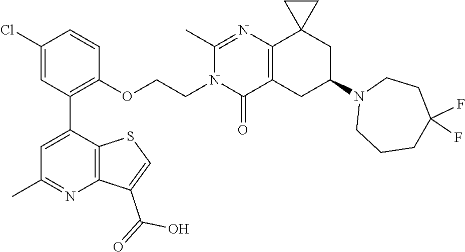 [SMILES: Cc1cc(-c2cc(Cl)ccc2OCCn2c(C)nc3c(c2=O)C[C@H](N2CCCC(F)(F)CC2)CC32CC2)c2scc(C(=O)O)c2n1] |
| 546 | 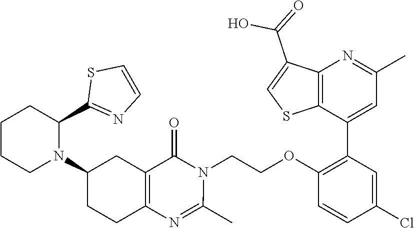 [SMILES: Cc1cc(-c2cc(Cl)ccc2OCCn2c(C)nc3c(c2=O)C[C@H](N2CCCC[C@H]2c2nccs2)CC3)c2scc(C(=O)O)c2n1] |
| 547 | 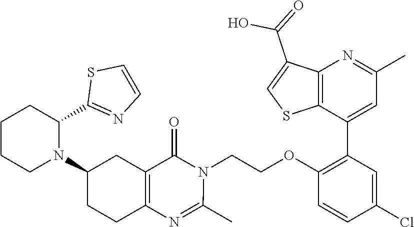 [SMILES: Cc1cc(-c2cc(Cl)ccc2OCCn2c(C)nc3c(c2=O)C[C@H](N2CCCC[C@@H]2c2nccs2)CC3)c2scc(C(=O)O)c2n1] |
| 548 | 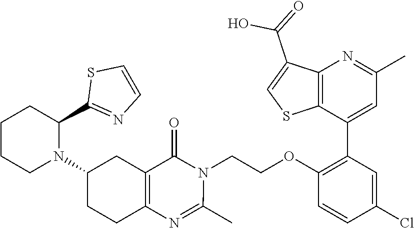 [SMILES: Cc1cc(-c2cc(Cl)ccc2OCCn2c(C)nc3c(c2=O)C[C@@H](N2CCCC[C@H]2c2nccs2)CC3)c2scc(C(=O)O)c2n1] |
| 549 | 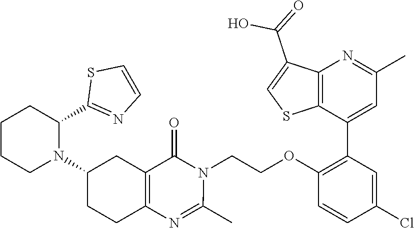 [SMILES: Cc1cc(-c2cc(Cl)ccc2OCCn2c(C)nc3c(c2=O)C[C@@H](N2CCCC[C@@H]2c2nccs2)CC3)c2scc(C(=O)O)c2n1] |
| 550 | 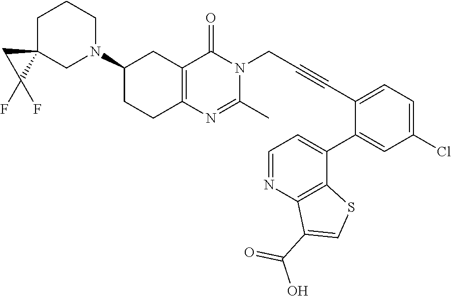 [SMILES: Cc1nc2c(c(=O)n1CC#Cc1ccc(Cl)cc1-c1ccnc3c(C(=O)O)csc13)C[C@H](N1CCC[C@@]3(C1)CC3(F)F)CC2] |
| 551 | 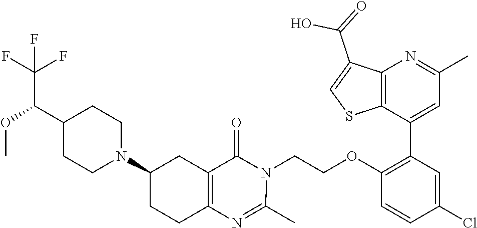 [SMILES: CO[C@@H](C1CCN([C@@H]2CCc3nc(C)n(CCOc4ccc(Cl)cc4-c4cc(C)nc5c(C(=O)O)csc45)c(=O)c3C2)CC1)C(F)(F)F] |
| 552 | 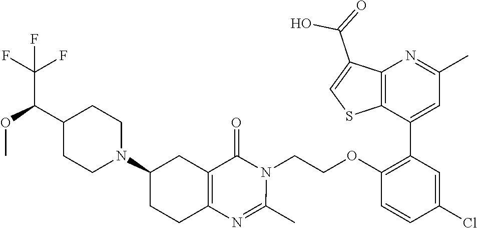 [SMILES: CO[C@H](C1CCN([C@@H]2CCc3nc(C)n(CCOc4ccc(Cl)cc4-c4cc(C)nc5c(C(=O)O)csc45)c(=O)c3C2)CC1)C(F)(F)F] |
| 553 | 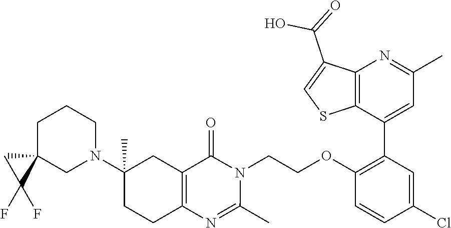 [SMILES: Cc1cc(-c2cc(Cl)ccc2OCCn2c(C)nc3c(c2=O)C[C@](C)(N2CCC[C@]4(C2)CC4(F)F)CC3)c2scc(C(=O)O)c2n1] |
| 554 | 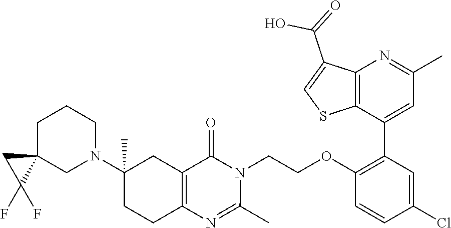 [SMILES: Cc1cc(-c2cc(Cl)ccc2OCCn2c(C)nc3c(c2=O)C[C@](C)(N2CCC[C@@]4(C2)CC4(F)F)CC3)c2scc(C(=O)O)c2n1] |
| 555 | 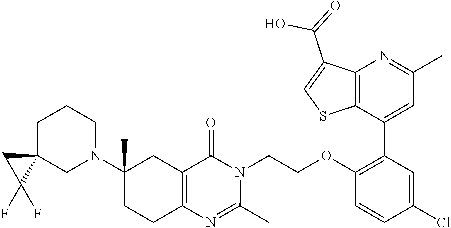 [SMILES: Cc1cc(-c2cc(Cl)ccc2OCCn2c(C)nc3c(c2=O)C[C@@](C)(N2CCC[C@@]4(C2)CC4(F)F)CC3)c2scc(C(=O)O)c2n1] |
| 556 | 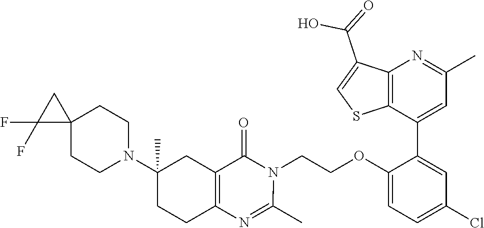 [SMILES: Cc1cc(-c2cc(Cl)ccc2OCCn2c(C)nc3c(c2=O)C[C@](C)(N2CCC4(CC2)CC4(F)F)CC3)c2scc(C(=O)O)c2n1] |
| 557 | 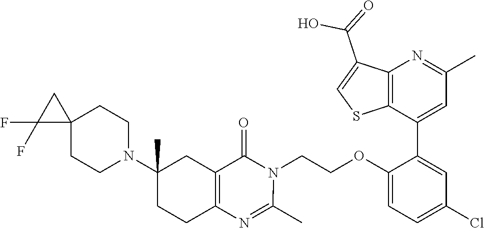 [SMILES: Cc1cc(-c2cc(Cl)ccc2OCCn2c(C)nc3c(c2=O)C[C@@](C)(N2CCC4(CC2)CC4(F)F)CC3)c2scc(C(=O)O)c2n1] |
| 558 | 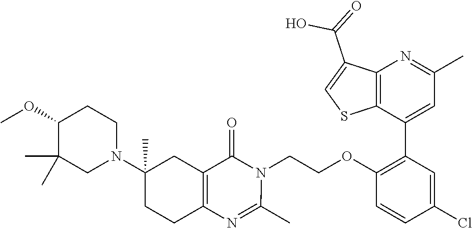 |
| 559 | 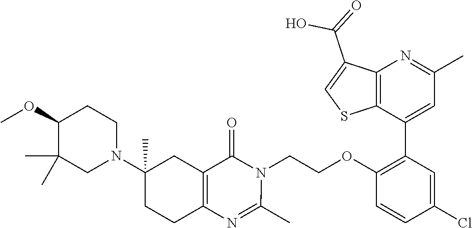 |
| 560 | 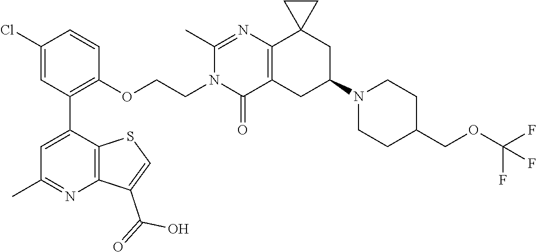 [SMILES: Cc1cc(-c2cc(Cl)ccc2OCCn2c(C)nc3c(c2=O)C[C@H](N2CCC(COC(F)(F)F)CC2)CC32CC2)c2scc(C(=O)O)c2n1] |
| 561 | 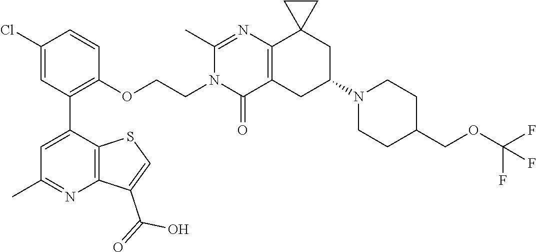 [SMILES: Cc1cc(-c2cc(Cl)ccc2OCCn2c(C)nc3c(c2=O)C[C@@H](N2CCC(COC(F)(F)F)CC2)CC32CC2)c2scc(C(=O)O)c2n1] |
| 562 | 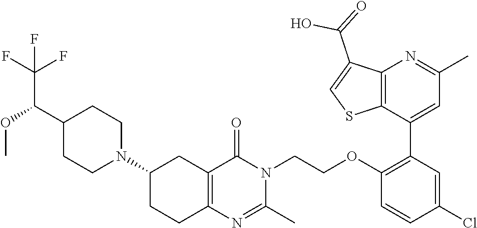 [SMILES: CO[C@@H](C1CCN([C@H]2CCc3nc(C)n(CCOc4ccc(Cl)cc4-c4cc(C)nc5c(C(=O)O)csc45)c(=O)c3C2)CC1)C(F)(F)F] |
| 563 | 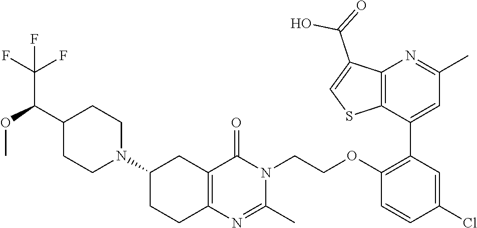 [SMILES: CO[C@H](C1CCN([C@H]2CCc3nc(C)n(CCOc4ccc(Cl)cc4-c4cc(C)nc5c(C(=O)O)csc45)c(=O)c3C2)CC1)C(F)(F)F] |
| 564 | 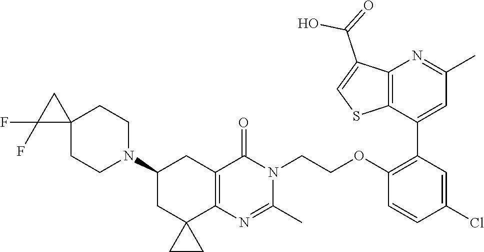 [SMILES: Cc1cc(-c2cc(Cl)ccc2OCCn2c(C)nc3c(c2=O)C[C@H](N2CCC4(CC2)CC4(F)F)CC32CC2)c2scc(C(=O)O)c2n1] |
| 565 | 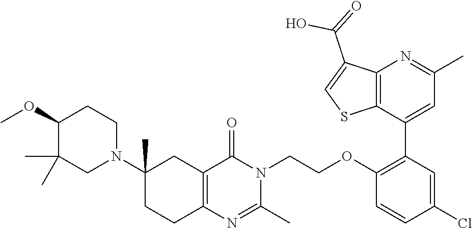 |
| 566 | 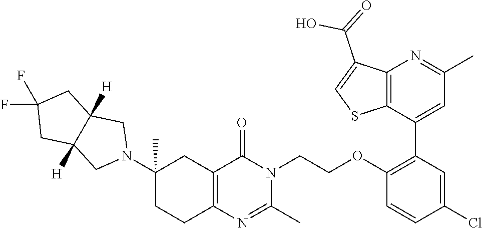 [SMILES: Cc1cc(-c2cc(Cl)ccc2OCCn2c(C)nc3c(c2=O)C[C@](C)(N2C[C@@H]4CC(F)(F)C[C@@H]4C2)CC3)c2scc(C(=O)O)c2n1] |
| 567 | 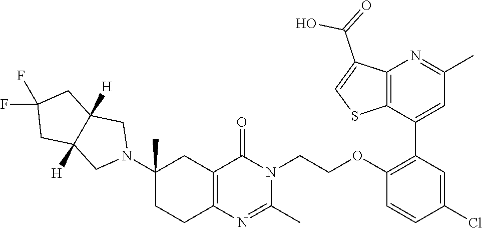 [SMILES: Cc1cc(-c2cc(Cl)ccc2OCCn2c(C)nc3c(c2=O)C[C@@](C)(N2C[C@@H]4CC(F)(F)C[C@@H]4C2)CC3)c2scc(C(=O)O)c2n1] |
| 568 | 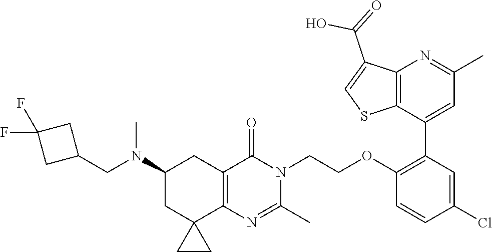 [SMILES: Cc1cc(-c2cc(Cl)ccc2OCCn2c(C)nc3c(c2=O)C[C@H](N(C)CC2CC(F)(F)C2)CC32CC2)c2scc(C(=O)O)c2n1] |
| 569 | 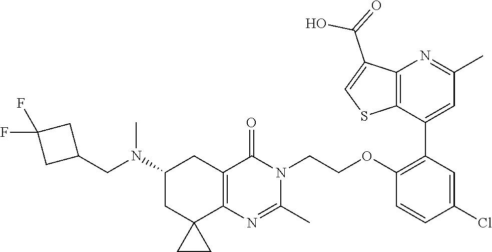 [SMILES: Cc1cc(-c2cc(Cl)ccc2OCCn2c(C)nc3c(c2=O)C[C@@H](N(C)CC2CC(F)(F)C2)CC32CC2)c2scc(C(=O)O)c2n1] |
| 570 | 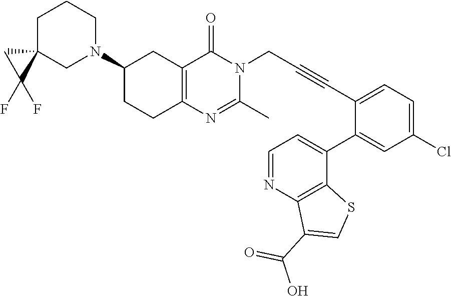 [SMILES: Cc1nc2c(c(=O)n1CC#Cc1ccc(Cl)cc1-c1ccnc3c(C(=O)O)csc13)C[C@H](N1CCC[C@]3(C1)CC3(F)F)CC2] |
| 571 | 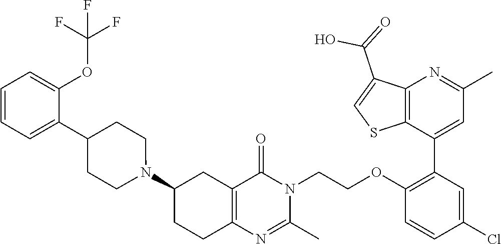 [SMILES: Cc1cc(-c2cc(Cl)ccc2OCCn2c(C)nc3c(c2=O)C[C@H](N2CCC(c4ccccc4OC(F)(F)F)CC2)CC3)c2scc(C(=O)O)c2n1] |
| 572 | 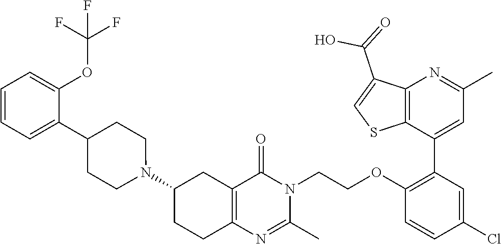 [SMILES: Cc1cc(-c2cc(Cl)ccc2OCCn2c(C)nc3c(c2=O)C[C@@H](N2CCC(c4ccccc4OC(F)(F)F)CC2)CC3)c2scc(C(=O)O)c2n1] |
| 573 | 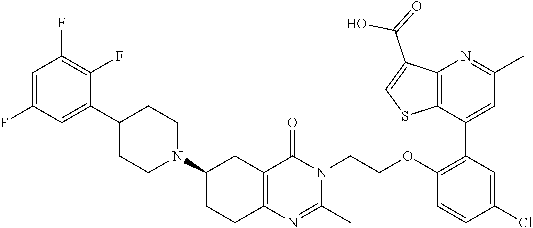 [SMILES: Cc1cc(-c2cc(Cl)ccc2OCCn2c(C)nc3c(c2=O)C[C@H](N2CCC(c4cc(F)cc(F)c4F)CC2)CC3)c2scc(C(=O)O)c2n1] |
| 574 | 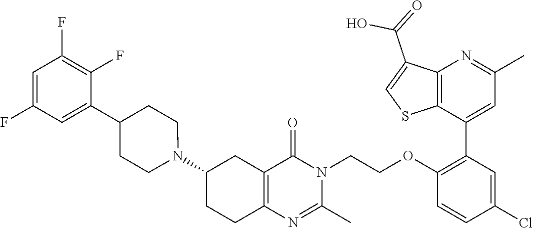 [SMILES: Cc1cc(-c2cc(Cl)ccc2OCCn2c(C)nc3c(c2=O)C[C@@H](N2CCC(c4cc(F)cc(F)c4F)CC2)CC3)c2scc(C(=O)O)c2n1] |
| 575 | 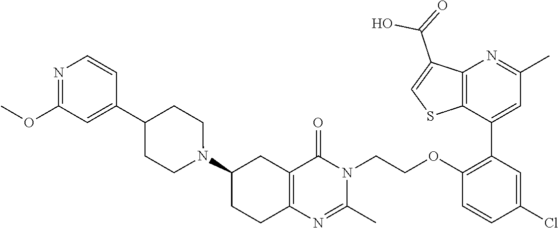 |
| 576 | 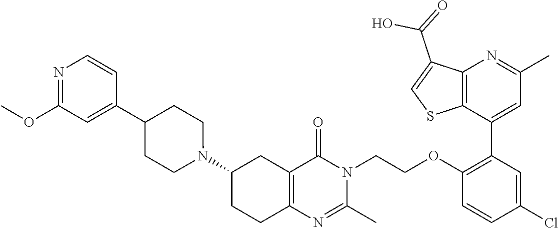 |
| 577 | 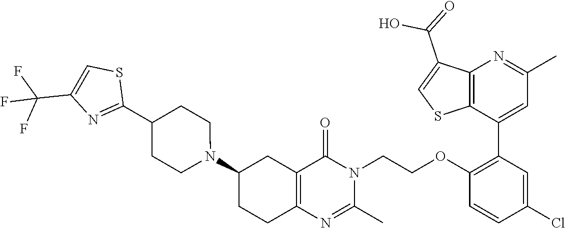 [SMILES: Cc1cc(-c2cc(Cl)ccc2OCCn2c(C)nc3c(c2=O)C[C@H](N2CCC(c4nc(C(F)(F)F)cs4)CC2)CC3)c2scc(C(=O)O)c2n1] |
| 578 | 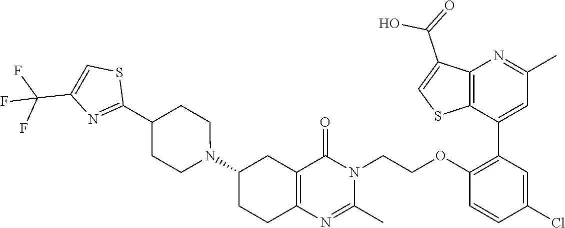 [SMILES: Cc1cc(-c2cc(Cl)ccc2OCCn2c(C)nc3c(c2=O)C[C@@H](N2CCC(c4nc(C(F)(F)F)cs4)CC2)CC3)c2scc(C(=O)O)c2n1] |
| 579 | 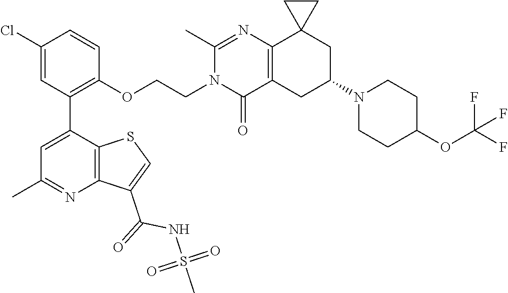 [SMILES: Cc1cc(-c2cc(Cl)ccc2OCCn2c(C)nc3c(c2=O)C[C@@H](N2CCC(OC(F)(F)F)CC2)CC32CC2)c2scc(C(=O)NS(C)(=O)=O)c2n1] |
| 580 | 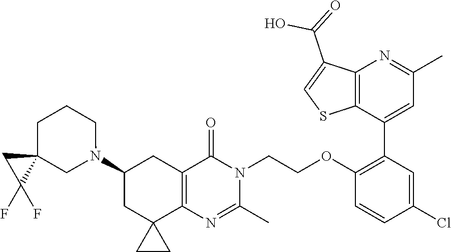 [SMILES: Cc1cc(-c2cc(Cl)ccc2OCCn2c(C)nc3c(c2=O)C[C@H](N2CCC[C@@]4(C2)CC4(F)F)CC32CC2)c2scc(C(=O)O)c2n1] |
| 581 | 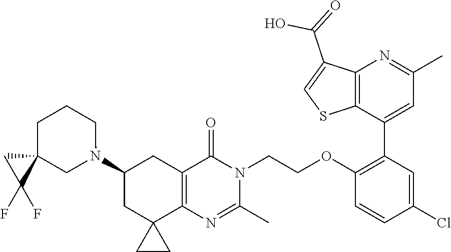 [SMILES: Cc1cc(-c2cc(Cl)ccc2OCCn2c(C)nc3c(c2=O)C[C@H](N2CCC[C@]4(C2)CC4(F)F)CC32CC2)c2scc(C(=O)O)c2n1] |
| 582 | 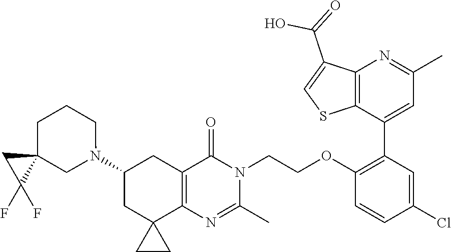 [SMILES: Cc1cc(-c2cc(Cl)ccc2OCCn2c(C)nc3c(c2=O)C[C@@H](N2CCC[C@@]4(C2)CC4(F)F)CC32CC2)c2scc(C(=O)O)c2n1] |
| 583 | 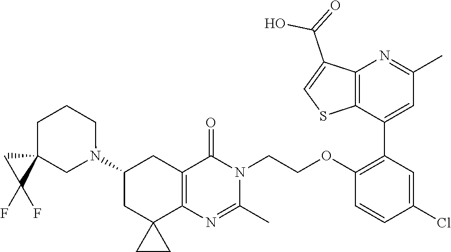 [SMILES: Cc1cc(-c2cc(Cl)ccc2OCCn2c(C)nc3c(c2=O)C[C@@H](N2CCC[C@]4(C2)CC4(F)F)CC32CC2)c2scc(C(=O)O)c2n1] |
| 584 | 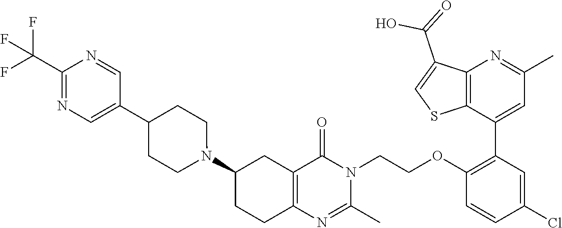 [SMILES: Cc1cc(-c2cc(Cl)ccc2OCCn2c(C)nc3c(c2=O)C[C@H](N2CCC(c4cnc(C(F)(F)F)nc4)CC2)CC3)c2scc(C(=O)O)c2n1] |
| 585 | 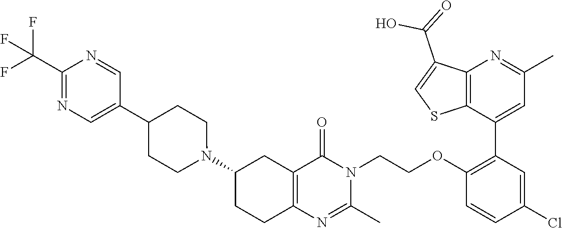 [SMILES: Cc1cc(-c2cc(Cl)ccc2OCCn2c(C)nc3c(c2=O)C[C@@H](N2CCC(c4cnc(C(F)(F)F)nc4)CC2)CC3)c2scc(C(=O)O)c2n1] |
| 586 | 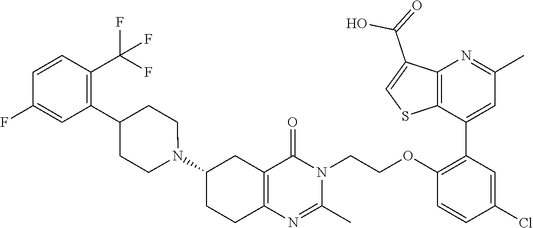 [SMILES: Cc1cc(-c2cc(Cl)ccc2OCCn2c(C)nc3c(c2=O)C[C@@H](N2CCC(c4cc(F)ccc4C(F)(F)F)CC2)CC3)c2scc(C(=O)O)c2n1] |
| 587 | 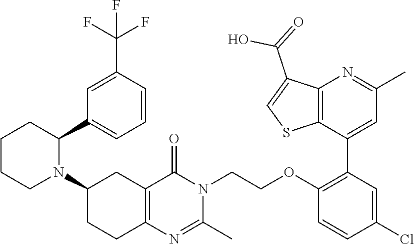 [SMILES: Cc1cc(-c2cc(Cl)ccc2OCCn2c(C)nc3c(c2=O)C[C@H](N2CCCC[C@H]2c2cccc(C(F)(F)F)c2)CC3)c2scc(C(=O)O)c2n1] |
| 588 | 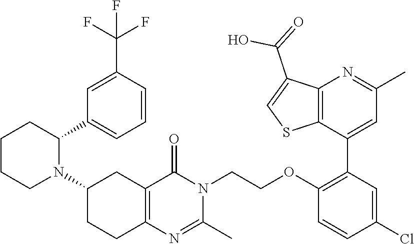 [SMILES: Cc1cc(-c2cc(Cl)ccc2OCCn2c(C)nc3c(c2=O)C[C@@H](N2CCCC[C@@H]2c2cccc(C(F)(F)F)c2)CC3)c2scc(C(=O)O)c2n1] |
| 589 | 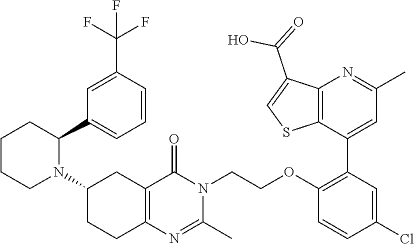 [SMILES: Cc1cc(-c2cc(Cl)ccc2OCCn2c(C)nc3c(c2=O)C[C@@H](N2CCCC[C@H]2c2cccc(C(F)(F)F)c2)CC3)c2scc(C(=O)O)c2n1] |
| 590 | 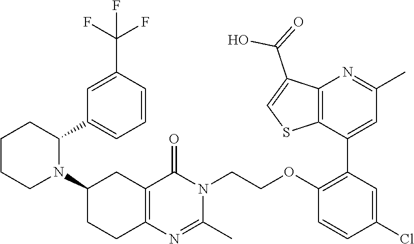 [SMILES: Cc1cc(-c2cc(Cl)ccc2OCCn2c(C)nc3c(c2=O)C[C@H](N2CCCC[C@@H]2c2cccc(C(F)(F)F)c2)CC3)c2scc(C(=O)O)c2n1] |
| 591 | 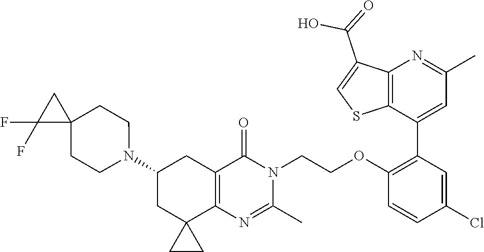 [SMILES: Cc1cc(-c2cc(Cl)ccc2OCCn2c(C)nc3c(c2=O)C[C@@H](N2CCC4(CC2)CC4(F)F)CC32CC2)c2scc(C(=O)O)c2n1] |
| 592 | 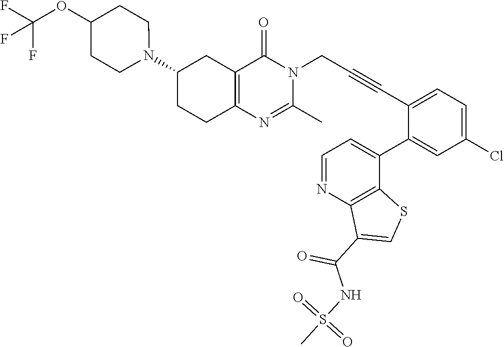 [SMILES: Cc1nc2c(c(=O)n1CC#Cc1ccc(Cl)cc1-c1ccnc3c(C(=O)NS(C)(=O)=O)csc13)C[C@@H](N1CCC(OC(F)(F)F)CC1)CC2] |
| 593 | 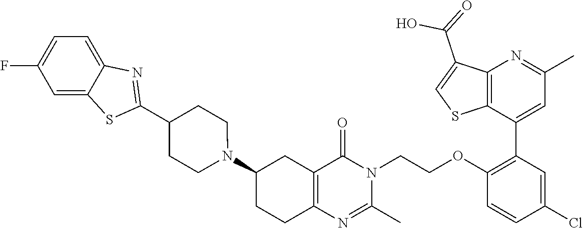 [SMILES: Cc1cc(-c2cc(Cl)ccc2OCCn2c(C)nc3c(c2=O)C[C@H](N2CCC(c4nc5ccc(F)cc5s4)CC2)CC3)c2scc(C(=O)O)c2n1] |
| 594 | 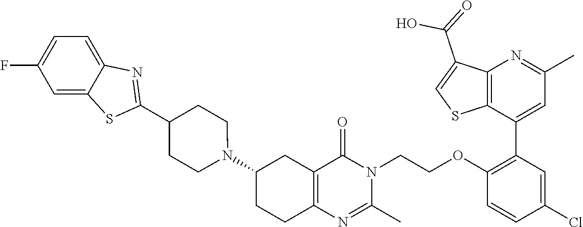 [SMILES: Cc1cc(-c2cc(Cl)ccc2OCCn2c(C)nc3c(c2=O)C[C@@H](N2CCC(c4nc5ccc(F)cc5s4)CC2)CC3)c2scc(C(=O)O)c2n1] |
| 595 | 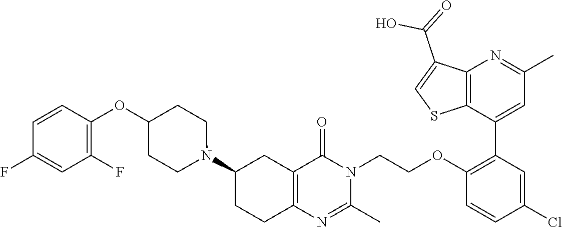 [SMILES: Cc1cc(-c2cc(Cl)ccc2OCCn2c(C)nc3c(c2=O)C[C@H](N2CCC(Oc4ccc(F)cc4F)CC2)CC3)c2scc(C(=O)O)c2n1] |
| 596 | 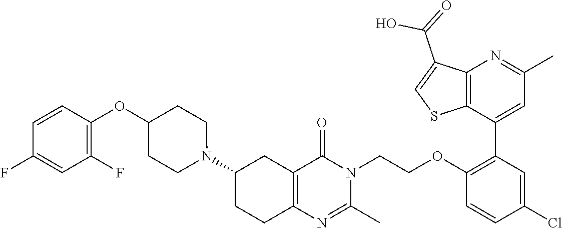 [SMILES: Cc1cc(-c2cc(Cl)ccc2OCCn2c(C)nc3c(c2=O)C[C@@H](N2CCC(Oc4ccc(F)cc4F)CC2)CC3)c2scc(C(=O)O)c2n1] |
| 597 | 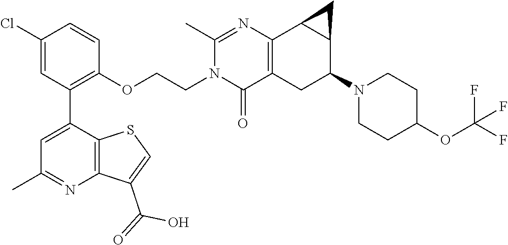 [SMILES: Cc1cc(-c2cc(Cl)ccc2OCCn2c(C)nc3c(c2=O)C[C@H](N2CCC(OC(F)(F)F)CC2)[C@@H]2C[C@H]32)c2scc(C(=O)O)c2n1] |
| 598 | 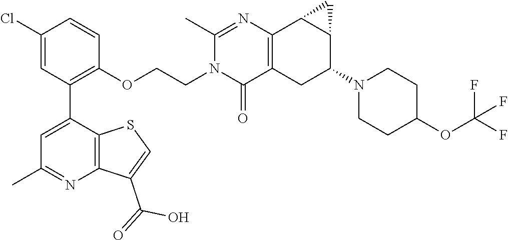 [SMILES: Cc1cc(-c2cc(Cl)ccc2OCCn2c(C)nc3c(c2=O)C[C@@H](N2CCC(OC(F)(F)F)CC2)[C@H]2C[C@@H]32)c2scc(C(=O)O)c2n1] |
| 599 | 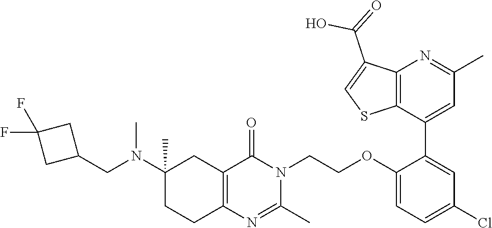 [SMILES: Cc1cc(-c2cc(Cl)ccc2OCCn2c(C)nc3c(c2=O)C[C@](C)(N(C)CC2CC(F)(F)C2)CC3)c2scc(C(=O)O)c2n1] |
| 600 | 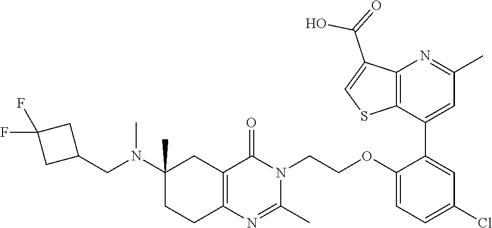 [SMILES: Cc1cc(-c2cc(Cl)ccc2OCCn2c(C)nc3c(c2=O)C[C@@](C)(N(C)CC2CC(F)(F)C2)CC3)c2scc(C(=O)O)c2n1] |
| 601 | 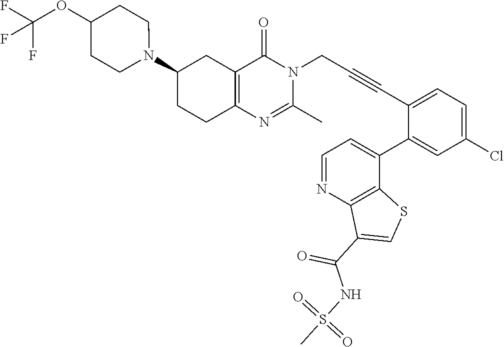 [SMILES: Cc1nc2c(c(=O)n1CC#Cc1ccc(Cl)cc1-c1ccnc3c(C(=O)NS(C)(=O)=O)csc13)C[C@H](N1CCC(OC(F)(F)F)CC1)CC2] |
| 602 | 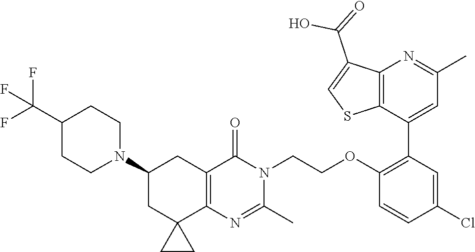 [SMILES: Cc1cc(-c2cc(Cl)ccc2OCCn2c(C)nc3c(c2=O)C[C@H](N2CCC(C(F)(F)F)CC2)CC32CC2)c2scc(C(=O)O)c2n1] |
| 603 | 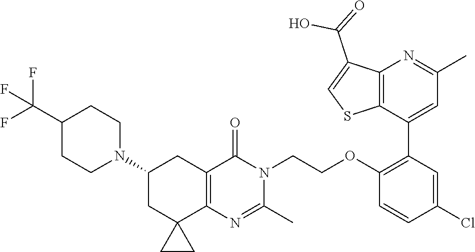 [SMILES: Cc1cc(-c2cc(Cl)ccc2OCCn2c(C)nc3c(c2=O)C[C@@H](N2CCC(C(F)(F)F)CC2)CC32CC2)c2scc(C(=O)O)c2n1] |
| 604 | 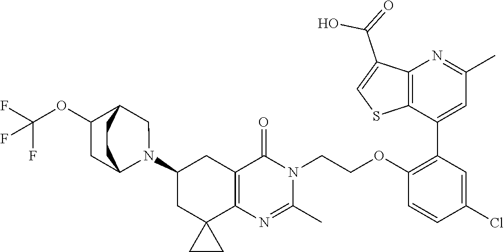 [SMILES: Cc1cc(-c2cc(Cl)ccc2OCCn2c(C)nc3c(c2=O)C[C@H](N2C[C@@H]4CC[C@H]2CC4OC(F)(F)F)CC32CC2)c2scc(C(=O)O)c2n1] |
| 605 | 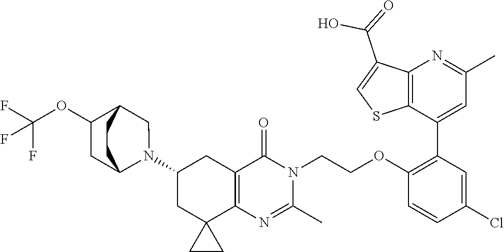 [SMILES: Cc1cc(-c2cc(Cl)ccc2OCCn2c(C)nc3c(c2=O)C[C@@H](N2C[C@@H]4CC[C@H]2CC4OC(F)(F)F)CC32CC2)c2scc(C(=O)O)c2n1] |
| 606 | 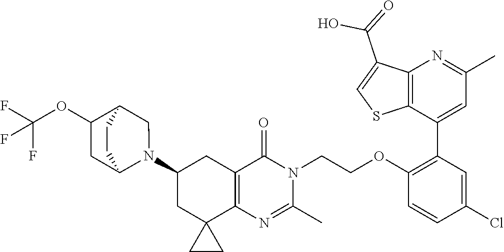 [SMILES: Cc1cc(-c2cc(Cl)ccc2OCCn2c(C)nc3c(c2=O)C[C@H](N2C[C@H]4CC[C@@H]2CC4OC(F)(F)F)CC32CC2)c2scc(C(=O)O)c2n1] |
| 607 | 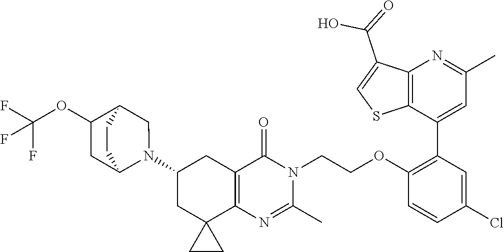 [SMILES: Cc1cc(-c2cc(Cl)ccc2OCCn2c(C)nc3c(c2=O)C[C@@H](N2C[C@H]4CC[C@@H]2CC4OC(F)(F)F)CC32CC2)c2scc(C(=O)O)c2n1] |
| 608 | 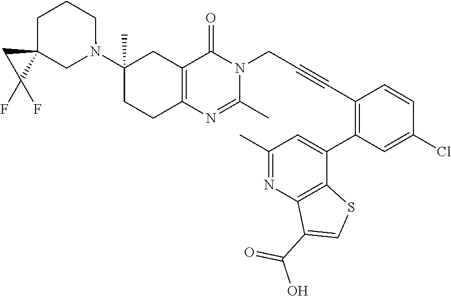 [SMILES: Cc1cc(-c2cc(Cl)ccc2C#CCn2c(C)nc3c(c2=O)C[C@](C)(N2CCC[C@@]4(C2)CC4(F)F)CC3)c2scc(C(=O)O)c2n1] |
| 609 | 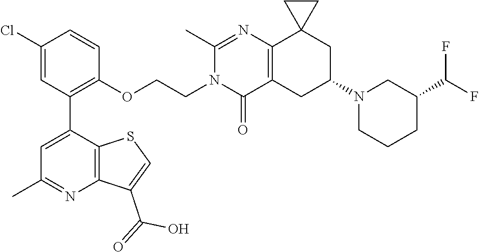 [SMILES: Cc1cc(-c2cc(Cl)ccc2OCCn2c(C)nc3c(c2=O)C[C@@H](N2CCC[C@@H](C(F)F)C2)CC32CC2)c2scc(C(=O)O)c2n1] |
| 610 | 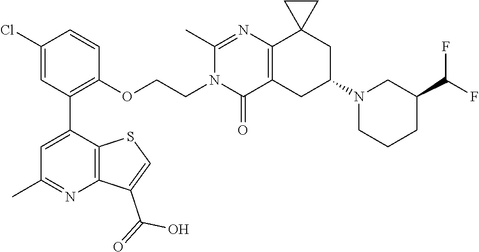 [SMILES: Cc1cc(-c2cc(Cl)ccc2OCCn2c(C)nc3c(c2=O)C[C@@H](N2CCC[C@H](C(F)F)C2)CC32CC2)c2scc(C(=O)O)c2n1] |
| 611 | 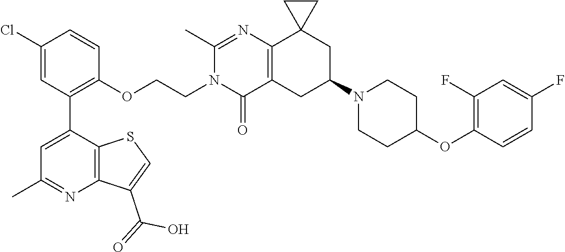 [SMILES: Cc1cc(-c2cc(Cl)ccc2OCCn2c(C)nc3c(c2=O)C[C@H](N2CCC(Oc4ccc(F)cc4F)CC2)CC32CC2)c2scc(C(=O)O)c2n1] |
| 612 | 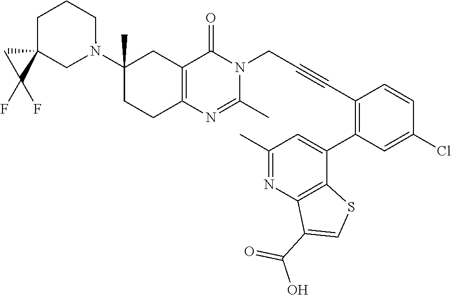 [SMILES: Cc1cc(-c2cc(Cl)ccc2C#CCn2c(C)nc3c(c2=O)C[C@@](C)(N2CCC[C@]4(C2)CC4(F)F)CC3)c2scc(C(=O)O)c2n1] |
| 613 | 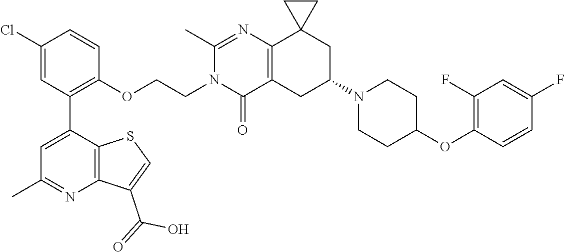 [SMILES: Cc1cc(-c2cc(Cl)ccc2OCCn2c(C)nc3c(c2=O)C[C@@H](N2CCC(Oc4ccc(F)cc4F)CC2)CC32CC2)c2scc(C(=O)O)c2n1] |
| 614 | 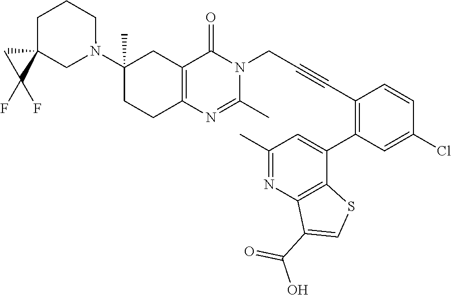 [SMILES: Cc1cc(-c2cc(Cl)ccc2C#CCn2c(C)nc3c(c2=O)C[C@](C)(N2CCC[C@]4(C2)CC4(F)F)CC3)c2scc(C(=O)O)c2n1] |
| 615 | 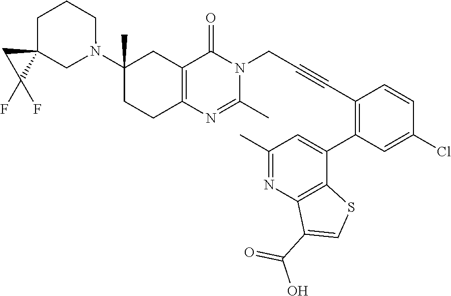 [SMILES: Cc1cc(-c2cc(Cl)ccc2C#CCn2c(C)nc3c(c2=O)C[C@@](C)(N2CCC[C@@]4(C2)CC4(F)F)CC3)c2scc(C(=O)O)c2n1] |
| 616 | 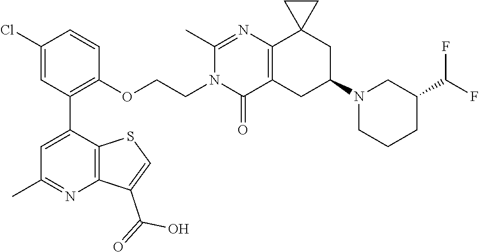 [SMILES: Cc1cc(-c2cc(Cl)ccc2OCCn2c(C)nc3c(c2=O)C[C@H](N2CCC[C@@H](C(F)F)C2)CC32CC2)c2scc(C(=O)O)c2n1] |
| 617 | 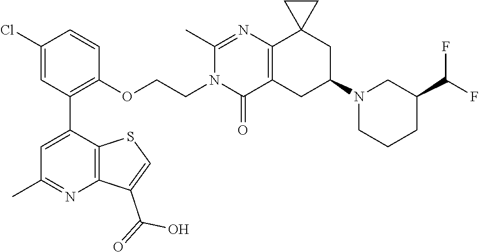 [SMILES: Cc1cc(-c2cc(Cl)ccc2OCCn2c(C)nc3c(c2=O)C[C@H](N2CCC[C@H](C(F)F)C2)CC32CC2)c2scc(C(=O)O)c2n1] |
| 618 | 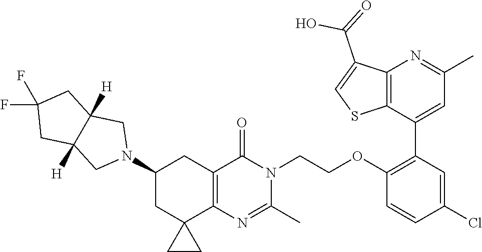 [SMILES: Cc1cc(-c2cc(Cl)ccc2OCCn2c(C)nc3c(c2=O)C[C@H](N2C[C@@H]4CC(F)(F)C[C@@H]4C2)CC32CC2)c2scc(C(=O)O)c2n1] |
| 619 | 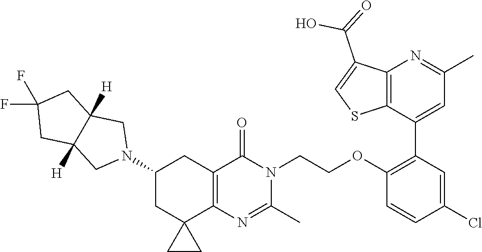 [SMILES: Cc1cc(-c2cc(Cl)ccc2OCCn2c(C)nc3c(c2=O)C[C@@H](N2C[C@@H]4CC(F)(F)C[C@@H]4C2)CC32CC2)c2scc(C(=O)O)c2n1] |
| 620 | 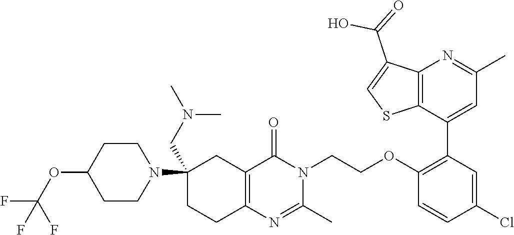 [SMILES: Cc1cc(-c2cc(Cl)ccc2OCCn2c(C)nc3c(c2=O)C[C@](CN(C)C)(N2CCC(OC(F)(F)F)CC2)CC3)c2scc(C(=O)O)c2n1] |
| 621 | 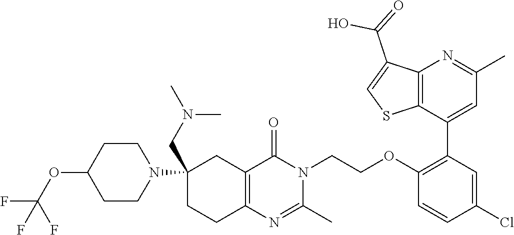 [SMILES: Cc1cc(-c2cc(Cl)ccc2OCCn2c(C)nc3c(c2=O)C[C@@](CN(C)C)(N2CCC(OC(F)(F)F)CC2)CC3)c2scc(C(=O)O)c2n1] |
| 622 | 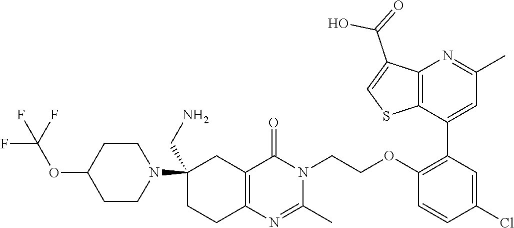 [SMILES: Cc1cc(-c2cc(Cl)ccc2OCCn2c(C)nc3c(c2=O)C[C@](CN)(N2CCC(OC(F)(F)F)CC2)CC3)c2scc(C(=O)O)c2n1] |
| 623 | 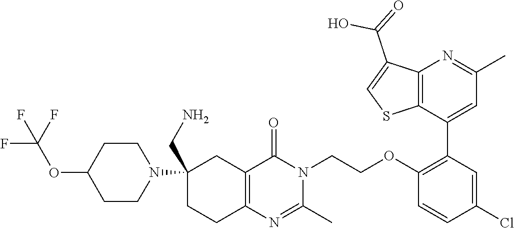 [SMILES: Cc1cc(-c2cc(Cl)ccc2OCCn2c(C)nc3c(c2=O)C[C@@](CN)(N2CCC(OC(F)(F)F)CC2)CC3)c2scc(C(=O)O)c2n1] |
| 624 | 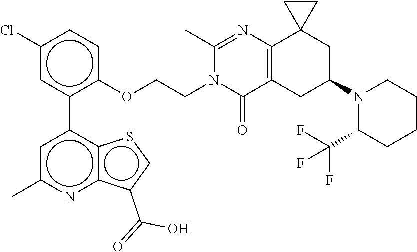 |
| 625 | 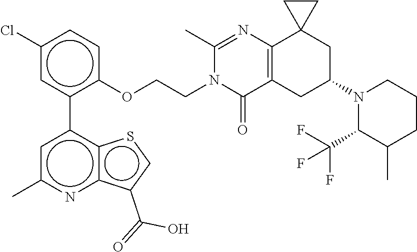 |
| 626 | 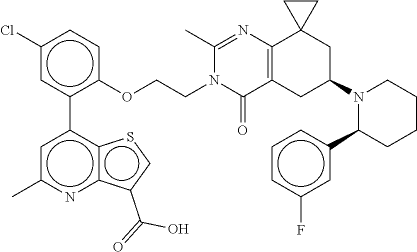 |
| 627 | 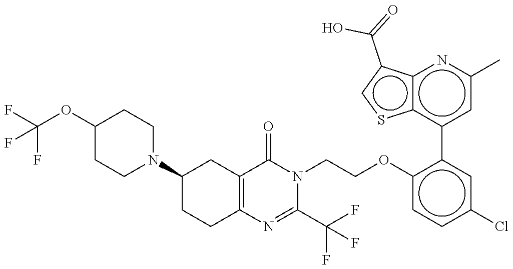 |
| 628 | 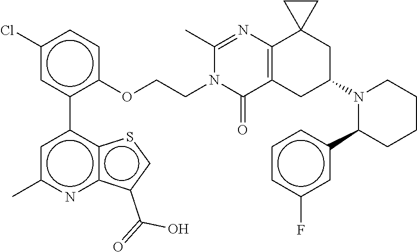 |
| 629 | 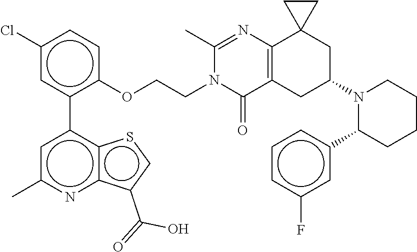 |
| 630 | 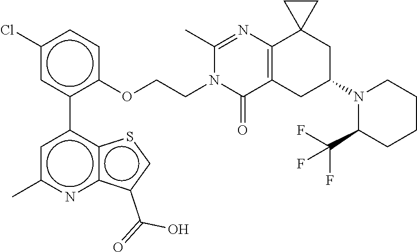 |
| 631 | 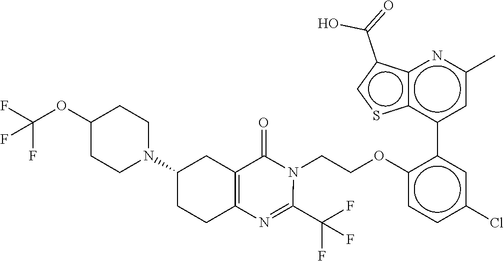 |
| 632 | 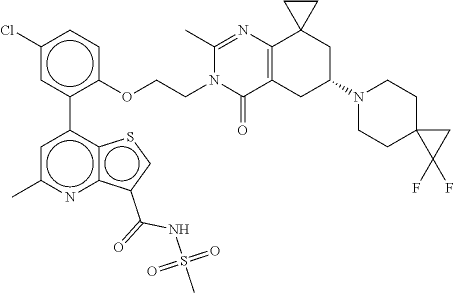 |
| 633 | 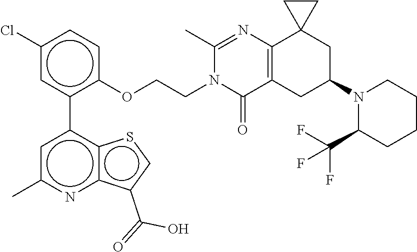 |
| 634 | 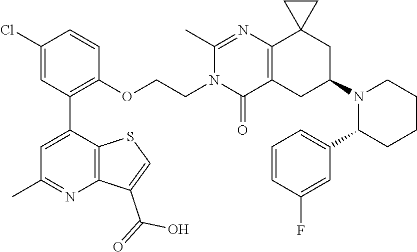 [SMILES: Cc1cc(-c2cc(Cl)ccc2OCCn2c(C)nc3c(c2=O)C[C@H](N2CCCC[C@@H]2c2cccc(F)c2)CC32CC2)c2scc(C(=O)O)c2n1] |

The compounds of the present disclosure possess advantageous characteristics, as compared to known compounds, such as known eIF4E inhibitors. For example, the compounds of the present disclosure display more potent eIF4E inhibition activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefore react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Pharmaceutical Compositions

In certain embodiments, the compound described herein is administered as a pure chemical. In certain embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, stereoisomer, or prodrug thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

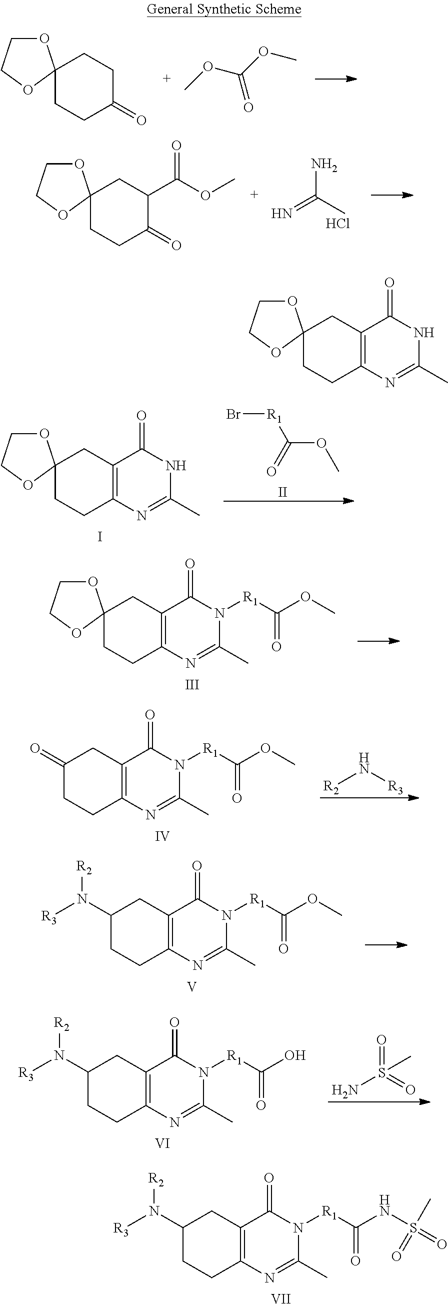

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka) (Pittsburgh, PA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Advanced Organic Chemistry: Reactions, Mechanisms and Structure”, 4th Ed., Wiley-Interscience, New York, 1992; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

For example, the affinity of compounds for proteins can be determined via a variety of biophysical assay formats, including surface plasmon resonance (SPR), isothermal titration calorimetry (ITC), mass spectrometry-based binding methods, and others. The competitive binding activity and potency of compounds for proteins can be assessed by a range of routine biochemical methods, including fluorescence polarization (FP), time-resolved fluorescence energy transfer (TR-FRET), and others. The functional effects of compounds on complex biological systems can be assessed in cell-free or cell lysate based assays such as in vitro translation, and others. The effects of compounds on specific target protein-related functions in cells (cellular activity and potency) can be assessed by a wide range of different methods, including reporter assays, immunoassays such as Western blot, cellular enzyme-linked immunoassays (ELISA), high-content imaging, and others. The effects of compounds on cellular phenotypes can be assayed by a range of methods, including those that measure cell proliferation, cell cycle progression, cell viability, cell death, cell migration, cell invasion, cell metabolism, and other cellular phenotypes.

### Methods of Use

In certain aspects, the present disclosure provides methods of inhibiting a protein in a subject or biological sample comprising administering the compound disclosed herein to the subject or contacting the biological sample with the compound disclosed herein.

In certain aspects, the present disclosure provides uses of the compound disclosed herein in the manufacture of a medicament for inhibiting a protein in a subject or biological sample.

In certain aspects, the present disclosure provides compounds disclosed herein for use in inhibiting a protein in a subject or biological sample.

In certain embodiments, the protein is eIF4E.

In certain aspects, the present disclosure provides methods of treating or preventing a disease or disorder a subject in need thereof, comprising administering to the subject the compound disclosed herein.

In certain aspects, the present disclosure provides uses of the compound disclosed herein in the manufacture of a medicament for treating or preventing a disease or disorder in a subject in need thereof.

In certain aspects, the present disclosure provides compounds disclosed herein for use in treating or preventing a disease or disorder in a subject in need thereof.

In certain embodiments, the disease or disorder is an eIF4E-mediated disease or disorder.

In certain embodiments, the disease or disorder is cancer.

In certain embodiments, the cancer includes, but is not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal carcinoma | endocrine gland | endodermal sinus | enteropathy- |
| | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ cell | medullary carcinoma |
| lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal zone | squamous cell | synovial sarcoma | Sezary's disease |
| lymphoma | carcinoma | | |
| small intestine cancer | squamous carcinoma | stomach cancer | T-cell lymphoma |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In certain embodiments, the cancer is a solid tumor. In certain embodiments, the cancer a hematological cancer. Exemplary hematological cancers include, but are not limited to, the cancers listed in Table B. In certain embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's macroglobulinemia | T-cell lymphoma |
| (WM) | |
| diffuse large B-cell lymphoma | mast cell leukemia |
| (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In certain embodiments, the cancer is colon cancer, gastric cancer, thyroid cancer, lung cancer, leukemia, B-cell lymphoma, T-cell lymphoma, hairy cell lymphoma, Hodgkin's lymphoma, non-Hodgkin's lymphoma, Burkitt's lymphoma, pancreatic cancer, melanoma, multiple melanoma, brain cancer, CNS cancer, renal cancer, prostate cancer, ovarian cancer, breast cancer, liver cancer, mesothelioma, rectal cancer, esophageal cancer, head and neck cancers, pancreatic cancer, uterine cancer, cervical cancer, or bladder cancer.

In certain embodiments, the disease or disorder is a non-cancer disease or disorder (e.g., an eIF4E-mediated non-cancer disease or disorder). In certain embodiments, the disease or disorder is cytokine related diseases, such as inflammatory diseases, allergies, or other conditions associated with proinflammatory cytokines. In certain embodiments, the disease or disorder is fibrotic diseases. In certain embodiments, the disease or disorder is a disease or disorder associated with the expression (or aberrant expression) and/or function (or dysfunction) of the eIF4E or in which the expression (or aberrant expression) and/or function (or dysfunction) of the eIF4E plays a role (e.g., in the initiation and/or development).

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. F. Eliel, Ed., Univ. of Notre Dame Press, Notre Dame, IN 1972).

The disclosure additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present disclosure. When describing the disclosure, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₈ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkylene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“C₁₋₁₀ heteroalkyl”). In certain embodiments, a heteroalkyl group is a saturated group having 1 to 9 carbon atoms and 1, 2, 3, or 4 heteroatoms (“C₁₋₉ heteroalkyl”). In certain embodiments, a heteroalkyl group is a saturated group having 1 to 8 carbon atoms and 1, 2, 3, or 4 heteroatoms (“C₁₋₈ heteroalkyl”). In certain embodiments, a heteroalkyl group is a saturated group having 1 to 7 carbon atoms and 1, 2, 3, or 4 heteroatoms (“C₁₋₇ heteroalkyl”). In certain embodiments, a heteroalkyl group is a group having 1 to 6 carbon atoms and 1, 2, or 3 heteroatoms (“C₁₋₆ heteroalkyl”). In certain embodiments, a heteroalkyl group is a saturated group having 1 to 5 carbon atoms and 1 or 2 heteroatoms (“C₁₋₅ heteroalkyl”). In certain embodiments, a heteroalkyl group is a saturated group having 1 to 4 carbon atoms and/or 2 heteroatoms (“C₁₋₄ heteroalkyl”). In certain embodiments, a heteroalkyl group is a saturated group having 1 to 3 carbon atoms and 1 heteroatom (“C₁₋₃ heteroalkyl”). In certain embodiments, a heteroalkyl group is a saturated group having 1 to 2 carbon atoms and 1 heteroatom (“C₁₋₂ heteroalkyl”). In certain embodiments, a heteroalkyl group is a saturated group having 1 carbon atom and 1 heteroatom (“C₁ heteroalkyl”). In certain embodiments, a heteroalkyl group is a saturated group having 2 to 6 carbon atoms and 1 or 2 heteroatoms (“C₂₋₆ heteroalkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted C₁₋₁₀ heteroalkyl. In certain embodiments, the heteroalkyl group is a substituted C₁₋₁₀ heteroalkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“C₂₋₁₀ heteroalkenyl”). In certain embodiments, a heteroalkenyl group has 2 to 9 carbon atoms at least one double bond, and 1, 2, 3, or 4 heteroatoms (“C₂₋₉ heteroalkenyl”). In certain embodiments, a heteroalkenyl group has 2 to 8 carbon atoms, at least one double bond, and 1, 2, 3, or 4 heteroatoms (“C₂₋₈ heteroalkenyl”). In certain embodiments, a heteroalkenyl group has 2 to 7 carbon atoms, at least one double bond, and 1, 2, 3, or 4 heteroatoms (“C₂₋₇ heteroalkenyl”). In certain embodiments, a heteroalkenyl group has 2 to 6 carbon atoms, at least one double bond, and 1, 2, or 3 heteroatoms (“C₂₋₆ heteroalkenyl”). In certain embodiments, a heteroalkenyl group has 2 to 5 carbon atoms, at least one double bond, and 1 or 2 heteroatoms (“C₂₋₅ heteroalkenyl”). In certain embodiments, a heteroalkenyl group has 2 to 4 carbon atoms, at least one double bond, and 1 or 2 heteroatoms (“C₂₋₄ heteroalkenyl”). In certain embodiments, a heteroalkenyl group has 2 to 3 carbon atoms, at least one double bond, and 1 heteroatom (“C₂₋₃ heteroalkenyl”). In certain embodiments, a heteroalkenyl group has 2 to 6 carbon atoms, at least one double bond, and 1 or 2 heteroatoms (“C₂₋₆ heteroalkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted C₂₋₁₀ heteroalkenyl. In certain embodiments, the heteroalkenyl group is a substituted C₂₋₁₀ heteroalkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“C₂₋₁₀ heteroalkynyl”). In certain embodiments, a heteroalkynyl group has 2 to 9 carbon atoms, at least one triple bond, and 1, 2, 3, or 4 heteroatoms (“C₂₋₉ heteroalkynyl”). In certain embodiments, a heteroalkynyl group has 2 to 8 carbon atoms, at least one triple bond, and 1, 2, 3, or 4 heteroatoms (“C₂₋₈ heteroalkynyl”). In certain embodiments, a heteroalkynyl group has 2 to 7 carbon atoms, at least one triple bond, and 1, 2, 3, or 4 heteroatoms (“C₂₋₇ heteroalkynyl”). In certain embodiments, a heteroalkynyl group has 2 to 6 carbon atoms, at least one triple bond, and 1, 2, or 3 heteroatoms (“C₂₋₆ heteroalkynyl”). In certain embodiments, a heteroalkynyl group has 2 to 5 carbon atoms, at least one triple bond, and 1 or 2 heteroatoms (“C₂₋₈ heteroalkynyl”). In certain embodiments, a heteroalkynyl group has 2 to 4 carbon atoms, at least one triple bond, and lor 2 heteroatoms (“C₂-4 heteroalkynyl”). In certain embodiments, a heteroalkynyl group has 2 to 3 carbon atoms, at least one triple bond, and 1 heteroatom (“C₂₋₃ heteroalkynyl”). In certain embodiments, a heteroalkynyl group has 2 to 6 carbon atoms, at least one triple bond, and 1 or 2 heteroatoms (“C₂₋₆ heteroalkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted C₂₋₁₀ heteroalkynyl. In certain embodiments, the heteroalkynyl group is a substituted C₂₋₁₀ heteroalkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

5-membered heteroaryl containing one heteroatom includes, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

In certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share one common bond with, one or more carbocyclyl groups, as defined above, wherein the point of attachment is on any of the fused rings. In such instances, the number of carbons designates the total number of carbons in the fused carbocyclyl ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on any of the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on any of the carbocyclyl rings in which the spiro structure is embedded.

“Bridged carbocyclyl” or “bridged carbocycle” refers to ring systems wherein the carbocyclyl group, as defined above, form bridged structure with, i.e., share more than one atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the bridged rings. When substitution is indicated, unless otherwise specified, substitution can occur on any of the carbocyclyl rings in which the bridged structure is embedded.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

In certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share one common bond with, one or more heterocyclyl or carbocyclyl groups, as defined above, wherein the point of attachment is on any of the fused rings. In such instances, the number of carbons designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than one atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the bridged rings.

“Hetero” when used to describe a compound or a group present on a compound means that one or more carbon atoms in the compound or group have been replaced by a nitrogen, oxygen, sulfur, boron, phosphorus, or silicon heteroatom, as valency permits. Hetero may be applied to any of the hydrocarbyl groups described above having from 1 to 5, and particularly from 1 to 3 heteroatoms.

“Alkoxy” as used herein, refers to the group —OR, wherein R is alkyl, carbocyclyl, or heterocyclyl as defined herein. C₁₋₆ alkoxy refers to the group —OR, wherein each R is C₁₋₆ alkyl, C₃₋₆ carbocyclyl, or 3- to 6-membered heterocyclyl, as defined herein. Exemplary C₁₋₆ alkyl, C₃₋₆ carbocyclyl, or 3- to 6-membered heterocyclyl is set forth above.

“Alkylamino” as used herein, refers to the group —NHR or —NR₂, wherein each R is independently alkyl, carbocyclyl, or heterocyclyl, as defined herein. C₁₋₆ alkylamino refers to the group —NHR or —NR₂, wherein each R is independently C₁₋₆ alkyl, C₃₋₆ carbocyclyl, or 3- to 6-membered heterocyclyl as defined herein. Exemplary C₁₋₆ alkyl, C₃₋₆ carbocyclyl, or 3- to 6-membered heterocyclyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g. p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The disclosure is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Examples

In order that the disclosure described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization

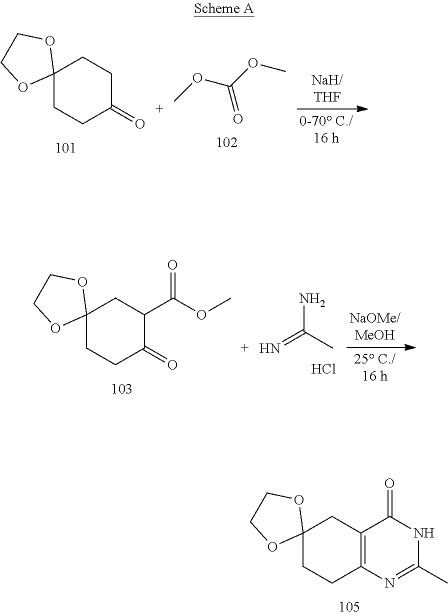

### Synthesis of 103

To a suspension of sodium hydride (60% dispersion in mineral oil) (1.92 g, 48.02 mmol, 60% purity) in THF (50 mL) were added 1,4-dioxaspiro[4.5]decan-8-one (101) (5 g, 32.01 mmol) followed by dimethyl carbonate (102) (8.65 g, 96.04 mmol, 8.09 mL, 99% purity) at 25° C. The reaction mixture was then heated at 70° C. for 16 hours. After completion of the reaction (as jugged by LC/MS & TLC), the reaction mixture was cooled to 25° C. and quenched with water (100 ml). The aqueous phase was extracted with EtOAc (2×100 ml). The combined organic layer was washed with brine, dried over anhydrous Na₂SO₄, filtered and filtrate was evaporated under reduced pressure to get the crude material. The crude was thus obtained was purified by silica gel column chromatography (SiO₂; 100-200 mesh, 15% EtOAc/Hexanes) to give methyl 8-oxidanylidene-1,4-dioxaspiro[4.5]decane-7-carboxylate (103) (5 g, 23.34 mmol, 72.91% yield) as an off white solid.

¹H NMR (400 MHz, DMSO-d6): δ 12.05 (s, 1H), 3.92 (s, 4H), 3.86 (s, 3H), 2.39 (t, J=6.76 Hz, 1H), 2.34 (s, 2H), 1.76 (t, J=6.72 Hz, 1H) ppm.

m/z calc. 214.08, found (M+1)=215.0.

### Synthesis of 105

To a stirred solution of methyl 8-oxidanylidene-1,4-dioxaspiro[4.5]decane-7-carboxylate (103) (5 g, 23.34 mmol) in MeOH (50 mL) was added NaOMe (25 mL, 4M) at 25° C. and the reaction mixture was stirred for 10 minutes. To this acetamidine HCl (104) (3.31 g, 35.01 mmol) was added at 25° C. and the reaction was stirred for 16 h at the same temperature. The solvent was removed under reduced pressure to get the crude material. The crude was dissolved in 1N HCl (40 mL) (pH˜7). The aqueous layer was extracted with 10% MeOH/DCM (50 mL×3), combined organic layer was dried over anhydrous sodium sulphate, concentrated under reduced pressure. The crude was purified by silica gel column chromatography (SiO₂; 100-200 mesh, 10% MeOH/DCM) to afford 2′-methylspiro[1,3-dioxolane-2,6′-3,5,7,8-tetrahydroquinazoline]-4′-one (105) (3.25 g, 14.62 mmol, 62.65% yield) as an off white solid.

¹H NMR (400 MHz, DMSO-d6) δ 12.24 (s, 1H), 3.91 (s, 4H), 2.62 (t, J=6.6 Hz, 2H), 2.46 (s, 2H), 2.23 (s, 3H), 1.81 (t, J=6.6 Hz, 2H) ppm.

MS calculated: 222.2; MS found: 222.8 (M+H).

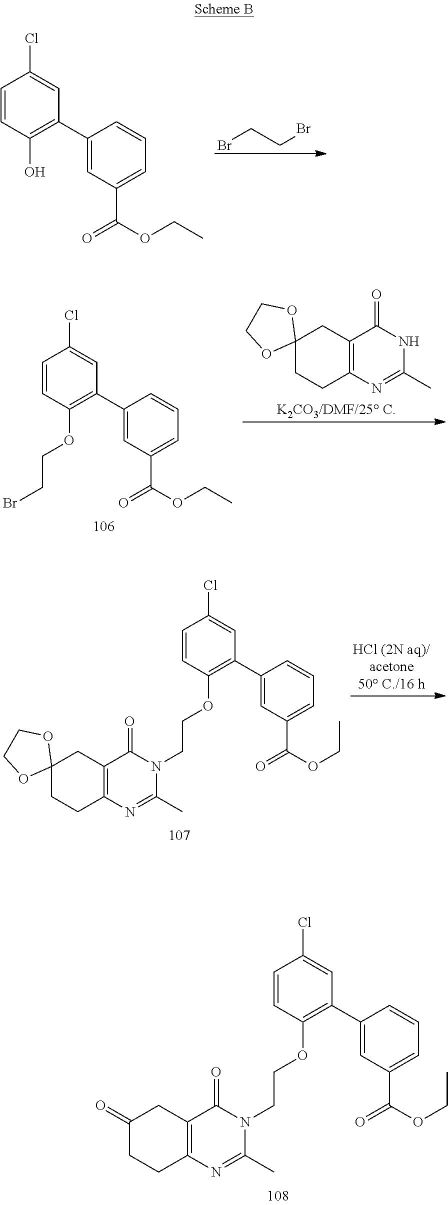

### Ethyl 3-[2-(2-bromanylethoxy)-5-chloranyl-phenyl]benzoate (106)

To a stirred solution of ethyl 3-(5-chloranyl-2-oxidanyl-phenyl)benzoate (4.5 g, 16.26 mmol) in Acetone (50 mL), K₂CO₃ (8.98 g, 65.05 mmol) was added and the mixture was stirred for 30 min. 1,2-Dibromoethane (13.75 g, 73.18 mmol, 6.31 mL) was added slowly and the reaction mixture was stirred at 60° C. for 16 h. The reaction mixture was passed through a celite pad, concentrated under reduced pressure and subjected to flash column chromatography (silica gel, 30% EtOAc/hexanes) to give 106 (4.0 g, 10.43 mmol, 64.11% yield). ¹H NMR (400 MHz, DMSO-d6-L): 8.186 (s, J=8.4, 1H), 7.959 (t, J=15.8, 1H), 7.663 (t, J=7.76 Hz, 1H), 7.566 (m, 1H), 7.42 (m, 2H), 7.17 (d, J=3.92, 1H), 4.32 (m, J=9.04 Hz, 4H), 3.73 (t, J=5.4 Hz, 2H), 1.335 (m, 3H) ppm.

### Synthesis of 107

To a stirred solution of 2′-methylspiro[1,3-dioxolane-2,6′-3,5,7,8-tetrahydroquinazoline]-4′-one (105) (400 mg, 1.80 mmol) in DMF (10 mL) were added potassium carbonate (746.25 mg, 5.40 mmol) followed by ethyl 3-[2-(2-bromanylethoxy)-5-chloranyl-phenyl]benzoate (106) (690.54 mg, 1.80 mmol) at 25° C. and the reaction mixture was stirred at 25° C. for 16 h. After completion of reaction, the reaction mixture was diluted with water (100 mL), extracted with EtOAc (2×100 mL). The combined organic layer was washed with brine, dried over anhydrous Na₂SO₄, filtered and filtrate was evaporated under reduced pressure. The crude thus obtained was purified by combi flash column chromatography (SiO₂; 12 g, 80% EtOAc) to get ethyl 3-[5-chloranyl-2-[2-(2′-methyl-4′-oxidanylidene-spiro[1,3-dioxolane-2,6′-7,8-dihydro-5H-quinazoline]-3′-yl)ethoxy]phenyl]benzoate (107) (360 mg, 685.73 μmol, 38.10% yield) as an off-white.

¹H NMR (400 MHz, DMSO-d₆) δ 7.94 (d, J=7.2 Hz, 1H), 7.88 (s, 1H), 7.57-7.51 (m, 2H), 7.40 (d, J=6.4 Hz, 1H), 7.31 (s, 1H), 7.18 (d, J=12 Hz, 1H), 4.35-4.30 (m, 2H), 4.24 (d, J=8 Hz, 2H), 4.18 (d, J=4 Hz, 2H), 3.90 (s, 4H), 2.59 (s, 2H), 2.49-2.45 (m, 2H), 2.05 (s, 3H), 1.82-1.80 (m, 2H), 1.32 (t, J=8 Hz, 3H).

LC-MS: 525.0 (M+H).

### Synthesis of 108

To a stirred solution of ethyl 3-[5-chloranyl-2-[2-(2′-methyl-4′-oxidanylidene-spiro[1,3-dioxolane-2,6′-7,8-dihydro-5H-quinazoline]-3′-yl)ethoxy]phenyl]benzoate (107) (500 mg, 952.40 μmol) in acetone (10 mL) was added 10% HCl in H2O (10 mL) at 25° C. and the reaction mixture was stirred at 55° C. for 16 h. After completion of reaction, the volatiles were removed under reduced pressure. The crude was dissolved in H2O and neutralised with saturated NaHCO₃ solution (50 mL). The aqueous layer was extracted with EtOAc. The combined organic layer was washed with brine, dried over anhydrous Na₂SO₄, filtered and filtrate was evaporated under reduced pressure. The crude thus obtained was purified by combi flash column chromatography (SiO₂; 12 g, 70% EtOAc) to afford ethyl 3-[5-chloranyl-2-[2-[2-methyl-4,6-bis(oxidanylidene)-7,8-dihydro-5H-quinazolin-3-yl]ethoxy]phenyl]benzoate (108) (290 mg, 602.99 μmol, 63.31% yield) as an off-white solid.

¹H NMR (400 MHz, DMSO-d₆) δ 7.93 (d, J=8 Hz, 1H), 7.88 (s, 1H), 7.58-7.50 (m, 2H), 7.41-7.39 (m, 1H), 7.30 (d, J=2.8 Hz, 1H), 7.19 (d, J=8.8 Hz, 1H), 4.32 (t, J=7.4 Hz, 2H), 4.27-4.22 (m, 4H), 3.11 (s, 2H), 2.84 (t, J=6.8 Hz, 2H), 2.53 (t, J=6.8 Hz, 2H), 2.07 (s, 3H), 1.33 (t, J=6.8 Hz, 3H).

LC-MS: 481.02 (M+H).

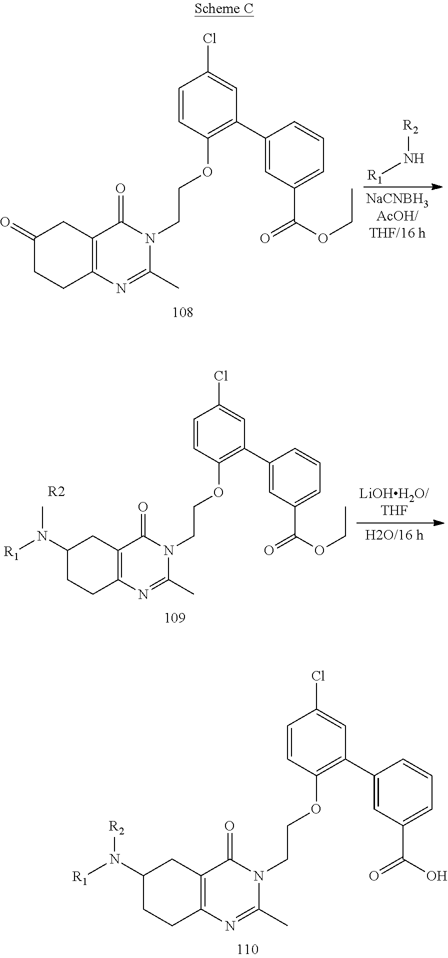

### Synthesis of 109

To a stirred solution of ethyl 3-[5-chloranyl-2-[2-[2-methyl-4,6-bis(oxidanylidene)-7,8-dihydro-5H-quinazolin-3-yl]ethoxy]phenyl]benzoate (108) (100 mg, 207.93 μmol) in MeOH (5 mL) were added AcOH (12.49 mg, 207.93 μmol) followed by NaBH₃CN (78.40 mg, 1.25 mmol) at 25° C. and the reaction mixture was stirred for 15 min at that temperature. To this was added N-methylmethanamine (46.87 mg, 1.04 mmol, 60.48 μL) at 0° C. and the reaction mixture was stirred for 16 h at 25° C. After completion of reaction, the reaction mixture was concentrated under reduced pressure and crude thus obtained diluted with sat NH4Cl solution (30 mL). The aqueous layer was extracted with EtOAc. The combined organic layer was washed with brine, dried over anhydrous Na₂SO₄, filtered and filtrate was evaporated under reduced pressure. The crude thus obtained was purified by combi flash column chromatography (SiO₂; 12 g, 5% MeOH/DCM) to afford ethyl 3-[5-chloranyl-2-[2-[6-[di(methyl)amino]-2-methyl-4-oxidanylidene-5,6,7,8-tetrahydroquinazolin-3-yl]ethoxy]phenyl]benzoate (109) (100 mg, 196.07 μmol, 94.30% yield) as an off-white solid.

¹H NMR (400 MHz, DMSO-d₆) δ 7.94 (d, J=7.2 Hz, 1H), 7.88 (s, 1H), 7.56-7.49 (m, 2H), 7.39 (t, J=8 Hz, 1H), 7.31 (s, 1H), 7.19 (d, J=9.2 Hz, 1H), 4.35-4.30 (m, 2H), 4.09-4.07 (m, 2H), 3.16 (d, J=4.8 Hz, 4H), 2.50 (s, 3H), 2.41 (s, 4H), 2.04 (s, 3H), 1.90 (s, 1H), 1.53 (d, J=6.8 Hz, 1H), 1.33 (t, J=6.8 Hz, 3H), 1.23 (s, 3H).

LC-MS: 510.1 (M+H).

### Synthesis of 110: (Compound 1)

To a stirred solution of ethyl 3-[5-chloranyl-2-[2-[6-[di(methyl)amino]-2-methyl-4-oxidanylidene-5,6,7,8-tetrahydroquinazolin-3-yl]ethoxy]phenyl]benzoate (109) (100 mg, 196.07 mol) in THF:H2O (7:3) (5 mL) was added LiOH·H2O (24.68 mg, 588.21 μmol, 16.35 μL) at 0° C. and the reaction mixture was stirred for 16 h at 25° C. After completion of reaction, the volatiles were removed under reduced pressure. The crude thus obtained was purified by reverse phase prep-HPLC to give 3-[5-chloranyl-2-[2-[6-[di(methyl)amino]-2-methyl-4-oxidanylidene-5,6,7,8-tetrahydroquinazolin-3-yl]ethoxy]phenyl]benzoic acid (110) (Compound 1) (30 mg, 61.71 μmol, 31.47% yield, 99.14% purity) as a white solid.

¹H NMR (400 MHz, MeOD): δ 7.94 (d, J=7.6 Hz, 1H), 7.75 (s, 1H), 7.41-7.34 (m, 2H), 7.32-7.29 (m, 1H), 7.20 (d, J=2.8 Hz, 1H), 7.13-7.11 (m, 1H), 4.38-4.23 (m, 4H), 3.49 (t, J=7.6 Hz, 1H), 2.90 (s, 7H), 2.78-2.74 (m, 2H), 2.70-2.68 (m, 1H), 2.17-2.15 (m. 2H), 2.06 (s, 3H).

MS calculated: 481.97; MS found: 482.2 (M+H).

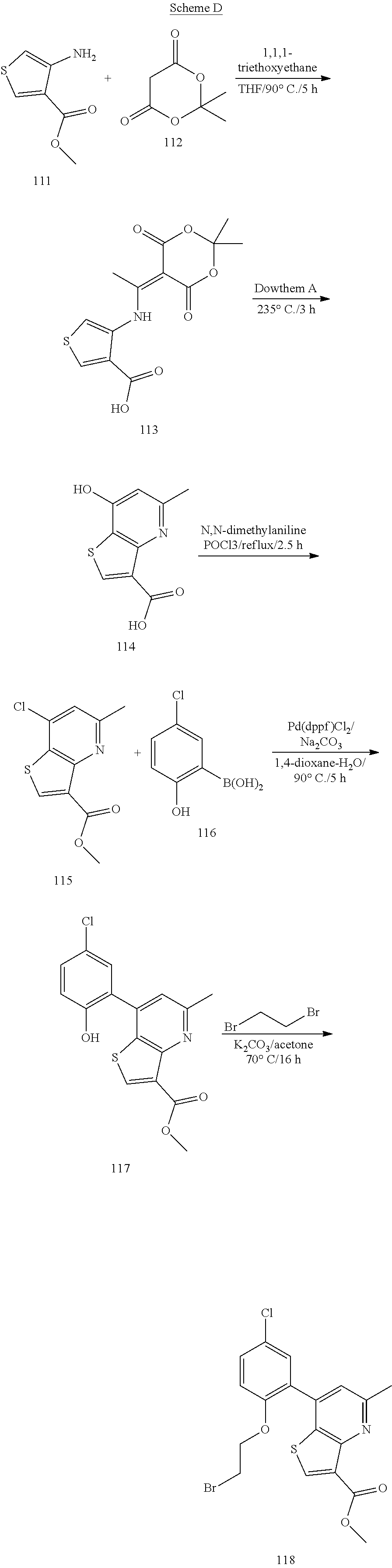

### Synthesis of 113

To a stirred solution of 2,2-di(methyl)-1,3-dioxane-4,6-dione (112) (7.06 g, 48.99 mmol) in 1,1,1-triethoxyethane (7.22 g, 44.53 mmol, 50 mL) was heated at 90° C. for 3 h. After consumption of starting (judged by TLC) the solvent was evaporated and the reaction mixture was dissolved in THF (50 mL). Then, methyl 4-azanylthiophene-3-carboxylate (111) (7 g, 44.53 mmol) was added to the reaction mixture and heating was continued at 90° C. for 2 h. After consumption of starting material, the reaction mixture was quenched with water and extracted with ethyl acetate (150 mL). The organic layer was washed with brine solution, dried over anhydrous sodium sulphate and concentrated under reduced pressure. Combiflash chromatography was done (SiO2, 120 g, 40% EtOAc/hexanes) to give methyl 4-[1-[2,2-di(methyl)-4,6-bis(oxidanylidene)-1,3-dioxan-5-ylidene]ethylamino]thiophene-3-carboxylate (113) (2 g, 6.15 mmol, 14%) as a yellow liquid.

¹H NMR (400 MHz, DMSO-d6) δ1.20 (m, 2H), 1.65 (s, 3H), 2.53 (m, 1H), 3.30 (s, 3H), 3.96 (m, 2H), 4.1 (m, 1H), 7.78 (d, J=12.3 Hz, 1H), 8.48 (d, J=3.28 Hz, 1H), 12.68 (s, 1H).

### Synthesis of 114

To a stirred solution of methyl 4-[1-[2,2-di(methyl)-4,6-bis(oxidanylidene)-1,3-dioxan-5-ylidene]ethylamino]thiophene-3-carboxylate (113) (20 g, 61.47 mmol) in Dowtherm (19.94 g, 61.47 mmol, 40 mL) was heated at 230° C. for 2 h. After completion of starting material (judged by TLC), the volatiles were removed under reduced pressure. The crude thus obtained was purified combiflash chromatography (SiO2, 120 g, 100% Ethyl acetate) to give methyl 5-methyl-7-oxidanyl-thieno[3,2-b]pyridine-3-carboxylate (114) (6 g, 26.88 mmol, 43.72% yield) as a brown solid.

1H NMR (400 MHz, DMSO-d6) δ 2.43 (s, 3H), 2.54 (s, 1H), 2.56 (s, 1H), 3.85 (s, 1H), 3.91 (s, 3H), 5.99 (s, 1H), 8.78 (s, 1H), 10.95 (s, 1H).

### Synthesis of 115

To a stirred solution of methyl 5-methyl-7-oxidanyl-thieno[3,2-b]pyridine-3-carboxylate (114) (3.5 g, 15.68 mmol) in toluene (50 mL) was added N,N-Dimethylaniline, 99% (15.20 g, 125.42 mmol, 15.90 mL) and cooled to 0° C. POCl₃ (3.60 g, 23.52 mmol) was added to the reaction mixture and heated at 120° C. for 2.5 h. After completion of starting material (judged by TLC), the solvent was evaporated under reduced pressure and crude thus obtained was purified by combiflash chromatography (SiO2, 120 g, 30% ethyl acetate/hexane) to get methyl 7-chloranyl-5-methyl-thieno[3,2-b]pyridine-3-carboxylate (115) (3.0 g, 12.41 mmol, 79.17% yield) as a white solid.

1H NMR (400 MHz, DMSO-d6) δ 8.93 (s, 1H), 7.57 (s, 1H), 3.86 (s, 3H), 2.63 (s, 3H).

ESI-MS: m/z calc. 241.0, found 243.0 (M+2)+.

### Synthesis of 117

To a stirred solution of methyl 7-chloranyl-5-methyl-thieno[3,2-b]pyridine-3-carboxylate (115) (3.0 g, 12.41 mmol) in dioxane (30 mL)& water (5 mL) were added (5-chloranyl-2-oxidanyl-phenyl)boronic acid (116) (2.14 g, 12.41 mmol) followed by Na₂CO₃ (3.95 g, 37.24 mmol) under Argon atmosphere and degassed for 10 min at 25° C. To this solution Pd(dppf)₂Cl₂ (907.35 mg, 1.24 mmol) was added and again degassed for 5 mins. Then, the reaction mixture was heated at 90° C. for 5 h. After completion of reaction (confirmed by LCMS), the reaction mixture was filtered through a celite bed and concentrated under reduced pressure. The crude thus obtained was purified by flash column chromatography (SiO2, 40 g, 30 to 35%: ethyl acetae/hexane) to give methyl 7-(5-chloranyl-2-oxidanyl-phenyl)-5-methyl-thieno[3,2-b]pyridine-3-carboxylate (117) (1.5 g, 4.49 mmol, 36.20% yield) as off white solid.

1H NMR (400 MHz, DMSO-d6) δ 10.26 (s, 1H), 8.84 (s, 1H), 7.38 (t, J=8.08 Hz 3H), 7.05 (d, J=8.68 Hz 1H), 3.87 (s, 3H), 2.66 (s, 3H).

ESI-MS: m/z calc. 333, found 334 (M+H)⁺.

### Synthesis of 118

To a stirred solution of methyl 7-(5-chloranyl-2-oxidanyl-phenyl)-5-methyl-thieno[3,2-b]pyridine-3-pcarboxylate (117) (1.2 g, 3.60 mmol) in acetone (25 mL) was added anhydrous potassium carbonate (1.49 g, 10.79 mmol, 650.91 μL) followed by 1,2-bis(bromanyl)ethane (5.40 g, 28.76 mmol, 2.48 mL) and the reaction mixture was refluxed at 70° C. for 16 h. After completion of reaction (as jugged by LC/MS only), the reaction mixture was cooled to 25° C. and filtered. The filtrate was evaporated under reduced pressure. The crude was thus obtained was purified by silica gel column chromatography (SiO₂; 40 g, 50% EtOAc/Hexanes) to give methyl 7-[2-(2-bromanylethoxy)-5-chloranyl-phenyl]-5-methyl-thieno[3,2-b]pyridine-3-carboxylate (118) (1.3 g, 2.95 mmol, 82.05% yield) as an off white solid.

1H-NMR (400 MHz, DMSO-d6) δ 8.84 (s, 1H), 7.57 (m, 2H), 7.42 (s, 1H), 7.30 (m, 1H), 4.36 (m, 2H), 3.93 (s, 3H), 3.62 (m, 2H), 2.66 (s, 3H).

ESI-MS: m/z calc 440.74; found 441.8 (M+1)⁻.

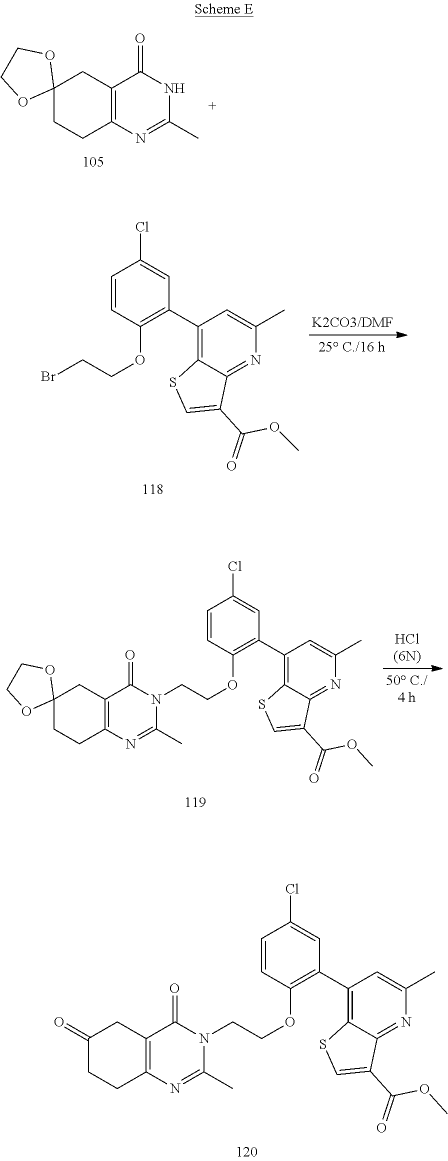 [SMILES: Cc1nc2c(c(=O)[nH]1)CC1(CC2)OCCO1 ; COC(=O)c1csc2c(-c3cc(Cl)ccc3OCCBr)cc(C)nc12 ; COC(=O)c1csc2c(-c3cc(Cl)ccc3OCCn3c(C)nc4c(c3=O)CC3(CC4)OCCO3)cc(C)nc12 ; COC(=O)c1csc2c(-c3cc(Cl)ccc3OCCn3c(C)nc4c(c3=O)CC(=O)CC4)cc(C)nc12]

### Synthesis of 119

To a stirred solution of 2′-methylspiro[1,3-dioxolane-2,6′-3,5,7,8-tetrahydroquinazoline]-4′-one (105) (1.5 g, 6.75 mmol) in DMF (15 mL) was added potassium carbonate (anhydrous) (2.79 g, 20.25 mmol) and the reaction was stirred for 10 min. To this was added methyl 7-[2-(2-bromanylethoxy)-5-chloranyl-phenyl]-5-methyl-thieno[3,2-b]pyridine-3-carboxylate (118) (2.97 g, 6.75 mmol) and the reaction was stirred at 25° C. for 16 h. After the completion of the reaction (checked by TLC & LC/MS), the reaction mixture was evaporated to dryness and then purified by combi flash column chromatography (SiO₂; 40 g, 4% MeOH/DCM) to give methyl 7-[5-chloranyl-2-[2-(2′-methyl-4′-oxidanylidene-spiro[1,3-dioxolane-2,6′-7,8-dihydro-5H-quinazoline]-3′-yl)ethoxy]phenyl]-5-methyl-thieno[3,2-b]pyridine-3-carboxylate (119) (1.6 g, 2.75 mmol, 40.73% yield) as an off white solid.

¹H-NMR (400 MHz, DMSO-d6) δ 8.68 (d, J=5.08 Hz 1H), 7.55-7.53 (m, 1H), 7.39 (d, J=2.16 Hz 1H), 7.31-7.24 (m, 2H), 4.06 (s, 2H), 3.88 (t, J=16.92 Hz 1H), 7.42 (d, J=2.6 Hz 10H), 2.76 (s, 3H), 2.68 (s, 2H), 1.88-1.82 (m, 2H), 1.57 (s, 3H) ppm.

MS calculated: 581; MS found: 582 (M+H).

### Synthesis of 120

A solution of the methyl 7-[5-chloranyl-2-[2-(2′-methyl-4′-oxidanylidene-spiro[1,3-dioxolane-2,6′-7,8-dihydro-5H-quinazoline]-3′-yl)ethoxy]phenyl]-5-methyl-thieno[3,2-b]pyridine-3-carboxylate (119) (1.6 g, 2.75 mmol) in HCl (15 mL, 6N) was allowed to stirred at 50° C. for 4 hours. The reaction mixture was poured into cold saturated sodium bicarbonate solution (100 ml). The aqueous layer was extracted with 10% MeOH/DCM (50 ml×3). The combined organic layer was dried over anhydrous sodium sulphate, concentrated under reduced pressure. The crude was purified by combi flash column chromatography (SiO₂; 12 g, 15% MeOH/DCM) to give methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (120) (890 mg, 1.65 mmol, 60.18% yield) as white solid.

¹H-NMR (400 MHz, DMSO-d6) δ 8.73 (s, 1H), 7.55 (dd, J=2.6 Hz, 8.96 Hz, 1H), 7.40 (d J=2.6 Hz, 1H), 7.31-7.23 (m, 2H), 4.37 (m, 2H), 4.10 (m, 2H), 3.88 (s, 3H), 3.11 (s, 2H), 2.79 (t, J=6.16 Hz, 2H), 2.71 (s, 3H), 2.58 (m, 1H), 1.60 (s, 3H) ppm.

m/z calc. 537.11; found: 538.2 (M+H)

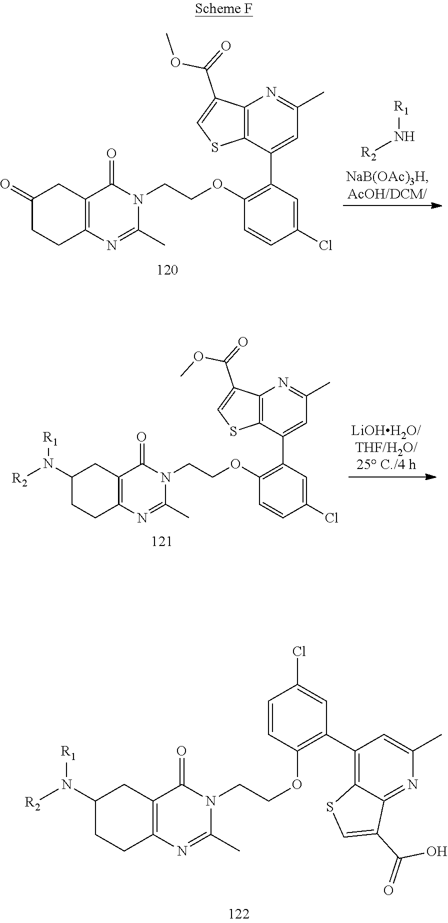

### Synthesis of 121

To a stirred solution of the compounds methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (120) (100 mg, 185.87 μmol) and N-methylmethanamine (12.57 mg, 278.81 μmol) in DCM (4 mL) was added catalytic AcOH at 0° C. The reaction mixture was then allowed to stirred for 2 h at 25° C. To this was added Na(OAc)₃BH (157.57 mg, 743.48 mol) at 0° C. and the reaction mixture was stirred at 25° C. for 5 h. After competition of reaction (as judged by TLC and LCMS), the excess solvents were evaporated under reduced pressure to get the crude material which was purified by silica gel column chromatography (SiO₂; 12 g, 15% MeOH/DCM) to give methyl 7-[5-chloranyl-2-[2-[6-[di(methyl)amino]-2-methyl-4-oxidanylidene-5,6,7,8-tetrahydroquinazolin-3-yl]ethoxy]phenyl]-5-methyl-thieno[3,2-b]pyri-dine-3-carboxylate (121) (81 mg, 142.83 μmol, 76.85% yield) as off white solid.

m/z calc. 566.2, found (M+1)=567.1.

### Synthesis of 122: (Compound 3)

To a stirred solution of the compound methyl 7-[5-chloranyl-2-[2-[6-[di(methyl)amino]-2-methyl-4-oxidanylidene-5,6,7,8-tetrahydroquinazolin-3-yl]ethoxy]phenyl]-5-methyl-thieno[3,2-b]pyridine-3-carboxylate (121) (78 mg, 137.54 μmol) in a mixture of THF (4 mL) and H₂O (1 mL) was added LiOH·H₂O (17.31 mg, 412.63 μmol) at 0° C. Then the reaction mixture was stirred at 25° C. for 16 h. After completion of reaction (as judged by TLC & LC/MS), the crude material was purified by reverse phase prep HPLC purification to get 7-[5-chloranyl-2-[2-[6-[di(methyl)amino]-2-methyl-4-oxidanylidene-5,6,7,8-tetrahydroquinazolin-3-yl]ethoxy]phenyl]-5-methyl-thieno[3,2-b]pyridine-3-carboxylic acid; 2,2,2-tris(fluorenyl)acetic acid (Compound 3) (0.032 g, 47.75 μmol, 34.72% yield, 99.54% purity) as white solid.

¹H NMR (400 MHz, DMSO-d6): δ 9.68 (brs, 1H), 8.82 (brs, 1H), 7.59 (d, J=8 Hz, 1H), 7.45 (s, 2H), 7.33 (d, J=8 Hz, 1H), 4.34 (brs, 2H), 4.09 (brs, 2H), 3.49 (brs, 1H), 2.85 (s, 6H), 2.78 (s, 2H), 2.55 (brs, 2H), 2.07 (brs, 1H), 1.78 (brs, 1H), 1.56 (s, 3H) ppm.

m/z calc. 552.1, found (M+1)=553.1.

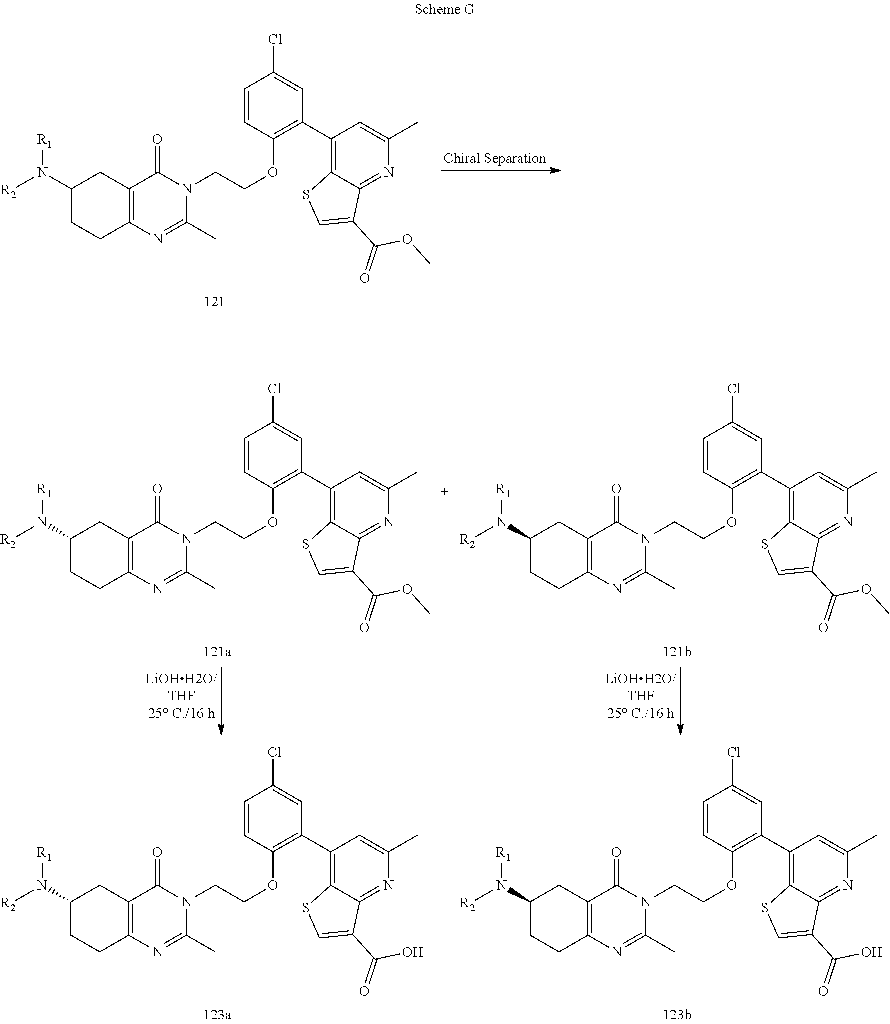

### Synthesis of 9a and 9b

This compound was synthesized in analogy to 7-[5-chloranyl-2-[2-[6-[di(methyl)amino]-2-methyl-4-oxidanylidene-5,6,7,8-tetrahydroquinazolin-3-yl]ethoxy]phenyl]-5-methyl-thieno[3,2-b]pyridine-3-carboxylate (121) from methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (120) (400 mg, 743.48 μmol) and N-methylmethanamine (50.28 mg, 1.12 mmol). Off white solid (340 mg, 76.85%%).

This racemic compound was separated by chiral prep HPLC to give methyl 7-[5-chloro-2-[2-[(3R,6S)-6-(dimethylamino)-2-methyl-4-oxo-5,6,7,8-tetrahydroquinazolin-3-yl]ethoxy]phenyl]-5-methyl-thieno[3,2-b]pyridine-3-carboxylate (121a, tentatively assigned) (140 mg, 246.87 μmol, 33.21% yield, 100% ee) as an off white solid and methyl 7-[5-chloro-2-[2-[(3S,6R)-6-(dimethylamino)-2-methyl-4-oxo-5,6,7,8-tetrahydroquinazolin-3-yl]ethoxy]phenyl]-5-methyl-thieno[3,2-b]pyridine-3-carboxylate (121b, tentatively assigned) (105 mg, 185.15 μmol, 24.90% yield, 99.56% ee) as an off white solid.

### Synthesis of 123a (Compound 13)

To a solution of 7-[5-chloro-2-[2-[(3R,6S)-6-(dimethylamino)-2-methyl-4-oxo-5,6,7,8-tetrahydroquinazolin-3-yl]ethoxy]phenyl]-5-methyl-thieno[3,2-b]pyridine-3-carboxylate (121a) (140 mg, 246.87 μmol) in a mixture of THF (5 ml) and water (1 ml) was added lithium hydroxide monohydrate (200 mg, 250 μmol), and the mixture was stirred at 25° C. for 16 h. Solvent was concentrated in vacuo. Resultant residue was purified by reverse phase prep HPLC to give 7-[5-chloro-2-[2-[(3R,6S)-6-(dimethylamino)-2-methyl-4-oxo-5,6,7,8-tetrahydroquinazolin-3-yl]ethoxy]phenyl]-5-methyl-thieno[3,2-b]pyridine-3-carboxylic acid; 2,2,2-trifluoroacetic acid (123a) (Compound 13) (83 mg, 123.92 μmol, 50.20% yield, 99.60% purity, 100% ee) as a white solid.

¹H NMR (400 MHz, DMSO-d6): δ 9.70 (brs, 1H), 8.82 (s, 1H), 7.59 (d, J=8 Hz, 1H), 7.45 (s, 2H), 7.33 (d, J=8 Hz, 1H), 4.34 (brs, 2H), 4.09 (brs, 2H), 3.49 (brs, 1H), 2.85 (s, 6H), 2.78 (s, 3H), 2.55 (brs, 2H), 2.07 (brs, 1H), 1.78-1.74 (m, 1H), 1.56 (s, 3H) ppm.

m/z calc. 552.1, found (M+1)=553.1.

### Synthesis of 123b (Compound 14)

To a solution of 7-[5-chloro-2-[2-[(3S,6R)-6-(dimethylamino)-2-methyl-4-oxo-5,6,7,8-tetrahydroquinazolin-3-yl]ethoxy]phenyl]-5-methyl-thieno[3,2-b]pyridine-3-carboxylate (121b) (105 mg, 185.15 μmol in a mixture of THF (5 ml) and water (1 ml) was added lithium hydroxide monohydrate (200 mg, 250 μmol), and the mixture was stirred at 25° C. for 16 h. Solvent was concentrated in vacuo. Resultant residue was purified by reverse phase prep HPLC to give 7-[5-chloro-2-[2-[(3S,6R)-6-(dimethylamino)-2-methyl-4-oxo-5,6,7,8-tetrahydroquinazolin-3-yl]ethoxy]phenyl]-5-methyl-thieno[3,2-b]pyridine-3-carboxylic acid; 2,2,2-trifluoroacetic acid (123b) (Compound 14) (57 mg, 85.25 μmol, 46.04% yield, 99.77% purity, 100% ee) as a white solid.

¹H NMR (400 MHz, DMSO-d6): δ 9.69 (brs, 1H), 8.82 (s, 1H), 7.59 (d, J=8 Hz, 1H), 7.45 (s, 2H), 7.33 (d, J=8 Hz, 1H), 4.34 (brs, 2H), 4.09 (brs, 2H), 3.49 (brs, 1H), 2.85 (s, 6H), 2.74 (s, 3H), 2.55 (brs, 2H), 2.18 (brs, 1H), 1.78-1.74 (m, 1H), 1.56 (s, 3H) ppm.

m/z calc. 552.1, found (M+1)=553.1.

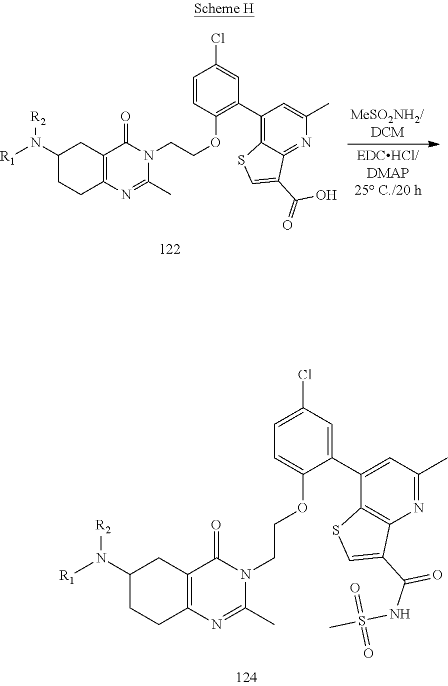

### Synthesis of 124 (Compound 39)

To a stirred solution of the compounds 7-[5-chloranyl-2-[2-[6-[di(methyl)amino]-2-methyl-4-oxidanylidene-5,6,7,8-tetrahydroquinazolin-3-yl]ethoxy]phenyl]-5-methyl-thieno[3,2-b]pyridine-3-carboxylic acid (122) (75 mg, 135.61 μmol) and MeSO₂NH₂ (32.25 mg, 339.02 mol) in DCM (4 mL) were added EDCI·HCl (51.99 mg, 271.21 μmol) followed by DMAP (41.42 mg, 339.02 μmol) at 0° C. and the reaction mixture was stirred at 25° C. for 20 h. After completion of reaction (as judged by TLC & LC/MS), the volatiles were removed under reduced pressure and the crude thus obtained was purified by reverse phase prep HPLC to get 7-[5-chloranyl-2-[2-[6-[di(methyl)amino]-2-methyl-4-oxidanylidene-5,6,7,8-tetrahydroquinazolin-3-yl]ethoxy]phenyl]-5-methyl-N-methylsulfonyl-thieno[3,2-b]pyridine-3-carboxamide; 2,2,2-tris(fluorenyl)acetic acid (124) (Compound 39) (18 mg, 23.93 μmol, 17.64% yield, 98.92% purity) as an off white solid.

¹H NMR (400 MHz, DMSO-d6): δ 13.00 (brs, 1H), 9.51 (brs, 1H), 8.96 (s, 1H), 7.57 (dd, J=9.2, 2.4 Hz, 1H), 7.43 (s, 1H), 7.42 (d, J=4.4 Hz, 1H), 7.32 (d, J=8.8 Hz, 1H), 4.36 (d, J=5.2 Hz, 2H), 4.14 (d, J=5.2 Hz, 2H), 3.49 (s, 3H), 2.86 (s, 6H), 2.83 (m, 1H), 2.77 (s, 3H), 2.58 (brs, 2H), 2.47-2.39 (m, 2H), 2.24-2.21 (m, 1H), 1.83-1.78 (m, 1H), 1.75 (s, 3H) ppm.

m/z calc. 629.15, found (M+1)=630.1 at RT 2.21.

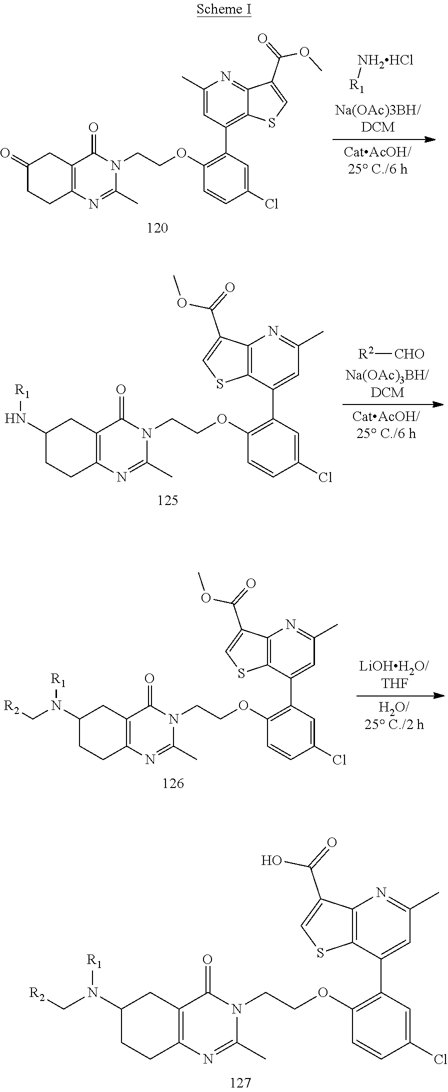

### Synthesis of 125

To a stirred solution of methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (120) (100 mg, 185.87 μmol) in DCM (5 mL) was added 3,3-bis(fluorenyl)cyclobutanamine (39.81 mg, 371.74 μmol) and the reaction mixture was stirred for 3 h at 25° C. To this was added sodium triacetoxyborohydride (196.97 mg, 929.34 μmol) at 0° C. portion wise and stirring was continued for 3 h at 25° C. After completion of reaction (as judged by TLC & LC/MS), the volatiles were removed under reduced pressure. The crude was purified by combi flash column chromatography (SiO₂; 12 g, 15% MeOH in DCM) to afford methyl 7-[2-[2-[6-[[3,3-bis(fluorenyl)cyclobutyl]amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (125) (100 mg, 158.95 μmol, 85.52% yield) as an off white solid.

1H NMR (400 MHz, DMSO-d6):8.70 (s, 1H), 7.55-7.53 (m, 1H), 7.39 (s, 1H), 7.31-7.26 (m, 2H), 4.32-4.30 (m, 2H), 4.05 (s, 2H), 3.88 (s, 3H), 2.78-2.72 (m, 3H), 2.66 (s, 3H), 1.59-1.57 (m, 4H), 1.23-1.21 (m, 4H) ppm.

m/z calc. 628, found 629.2 (M+1).

### Synthesis of 126

To a stirred solution of methyl 7-[2-[2-[6-[[3,3-bis(fluorenyl)cyclobutyl]amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (125) (100 mg, 158.95 μmol)) in dichloromethane (5 ml) were added cat. acetic acid followed by formalin (36 mg, 476.86 μmol, 33.05 μL, 40% purity) at 0° C. and the reaction mixture was stirred for 3 h at 25° C. To this was added sodium triacetoxyborohydride (135 mg, 635.81 μmol) at 0° C. portion wise and stirring was continued for 3 h at 25° C. After completion of reaction (as judged by TLC & LC/MS), the volatiles were removed under reduced pressure. The crude was purified by combi flash column chromatography (SiO₂; 12 g, 15% MeOH in DCM) to afford methyl 7-[2-[2-[6-[[3,3-bis(fluorenyl)cyclobutyl]-methyl-amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (126) (90 mg, 100.76 μmol, 63.39% yield, 72% purity) as an off white gum.

m/z calc. 642, found 643.4 (M+1).

### Synthesis of 127 (Compound 40)

To a stirred solution of methyl 7-[2-[2-[6-[[3,3-bis(fluorenyl)cyclobutyl]-methyl-amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (126) (90 mg, 139.94 μmol) in THF (1.5 ml), water (0.3 ml) & methanol (0.3 ml) was added lithium hydroxide; monohydrated (34 mg, 1.40 mmol) and the reaction mixture was stirred for 2 h at 25° C. After completion of reaction, the volatiles were removed in vacuo and the crude was purified by reverse phase preparative HPLC to get 7-[2-[2-[6-[[3,3-bis(fluorenyl)cyclobutyl]-methyl-amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylic acid; 2,2,2-tris(fluorenyl)acetic acid (Compound 40) (23.18 mg, 30.42 μmol, 21.74% yield, 97.53% purity) as a white sticky solid.

¹H NMR (400 MHz, DMSO-d6):8.81 (s, 1H), 7.59 (dd, J=8.8 Hz, 1H), 7.45 (m, 2H), 7.33 (d, J=8.8 Hz, 1H), 4.36 (m, 2H), 4.09 (m, 4H), 3.01 (m, 4H), 2.77-2.74 (m, 7H), 2.55 (s, 3H), 1.5 (s, 3H) ppm.

MS calculated: 628; MS found: 629.2 (M+H).

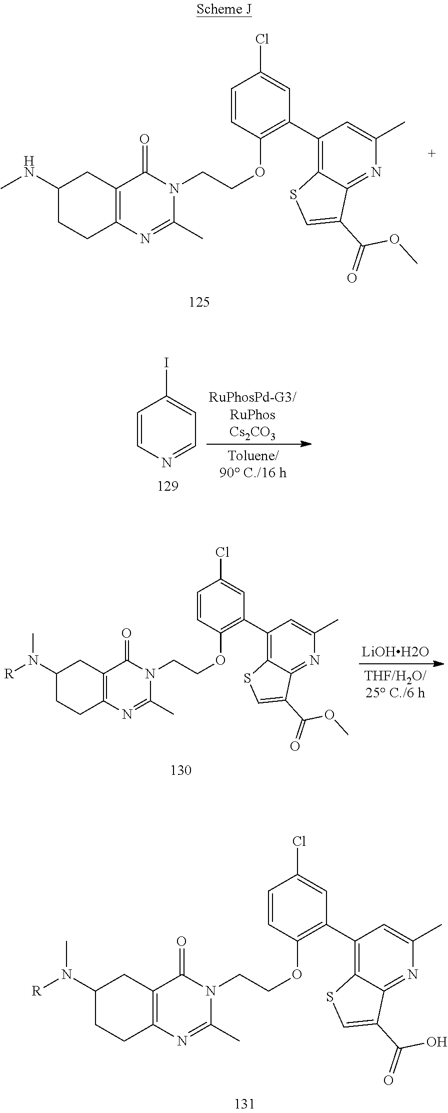

### Synthesis of 130

To a degassed solution of methyl 7-[5-chloranyl-2-[2-[2-methyl-6-(methylamino)-4-oxidanylidene-5,6,7,8-tetrahydroquinazolin-3-yl]ethoxy]phenyl]-5-methyl-thieno[3,2-b]pyridine-3-carboxylate (125) (150 mg, 271.21 μmol) in toluene (5 mL) were added 4-iodanylpyridine (4) (83.40 mg, 406.82 μmol), Cs₂CO₃ (353.66 mg, 1.08 mmol) at 25° C. To this was added RuPhos (12.64 mg, 27.12 μmol) followed by RuPhosPdG4 (23.09 mg, 27.12 μmol) at 25° C. and the reaction mixture was degassed with argon for 10 min The reaction mixture was heated at 90° C. for 16 h. After completion of reaction (as judged by TLC & LC/MS), the reaction mixture was cooled to 25° C. and filtered through celite pad. The filtrate was diluted with water and extracted with EtOAc. The combined organic layer was washed with brine, dried over anhydrous Na₂SO₄, filtered and filtrate was evaporated under reduced pressure. The crude was thus obtained was purified by silica gel combi flash column chromatography (SiO₂; 12 g, 4% MeOH/DCM) to get methyl 7-[5-chloranyl-2-[2-[2-methyl-6-[methyl(4-pyridyl)amino]-4-oxidanylidene-5,6,7,8-tetrahydroquinazolin-3-yl]ethoxy]phenyl]-5-methyl-thieno[3,2-b]pyridine-3-carboxylate (130) (110 mg, 174.56 μmol, 64.36% yield) as a brown solid.

m/z calc. 629.2, found (M+1)=630.3.

### Synthesis of 131 (Compound 47)

To a stirred solution of the compound methyl 7-[5-chloranyl-2-[2-[2-methyl-6-[methyl(4-pyridyl)amino]-4-oxidanylidene-5,6,7,8-tetrahydroquinazolin-3-yl]ethoxy]phenyl]-5-methyl-thieno[3,2-b]pyridine-3-carboxylate (130) (100 mg, 158.69 μmol) in a mixture of THF (2 mL) and water (500.00 μL) was added LiOH·H₂O (26.63 mg, 634.76 μmol, 17.64 μL) at 25° C. and the reaction mixture was stirred for 4 h at the same temperature. After completion of reaction (as judged by TLC & LC/MS), the reaction mixture was evaporated under reduced pressure. The crude thus obtained was purified by reverse phase prep-HPLC to afford 7-[5-chloranyl-2-[2-[2-methyl-6-[methyl(4-pyridyl)amino]-4-oxidanylidene-5,6,7,8-tetrahydroquinazolin-3-yl]ethoxy]phenyl]-5-methyl-thieno[3,2-b]pyridine-3-carboxylic acid; 2,2,2-tris(fluorenyl)acetic acid (Compound 47) (35 mg, 47.48 μmol, 29.92% yield, 99.06% purity) as a white sticky solid.

¹H NMR (400 MHz, DMSO-d6): δ 13.28 (brs, 1H), 8.85 (s, 1H), 8.20 (d, J=7.6, 2H), 7.57 (dd, J=8.8, 2.4 Hz, 1H), 7.44 (d, J=2.0 Hz, 1H), 7.41 (s, 1H), 7.32 (d, J=8.8 Hz, 1H), 7.16 (d, J=6.8 Hz, 1H), 4.34 (m, 3H), 4.14 (brs, 2H), 3.10 (s, 3H), 2.77 (s, 3H), 2.55 (s, 3H), 2.16-2.05 (m, 1H), 1.93-1.88 (m, 1H), 1.77 (s, 3H).

m/z calc. 615.17, found (M+1)=616.1 at RT 2.15.

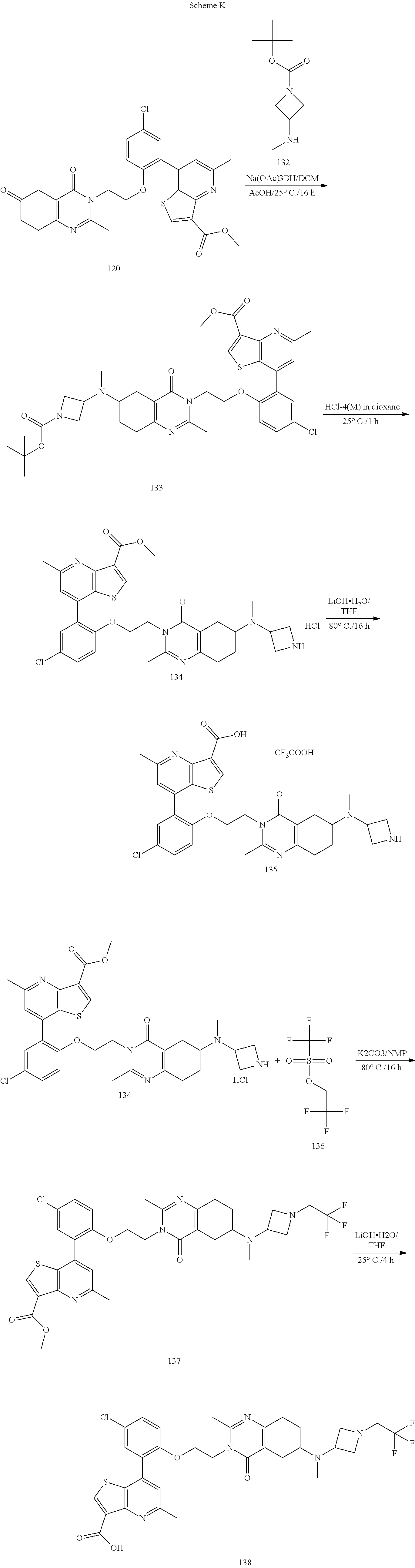

### Synthesis of 133

To a stirred solution of methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (120) (300 mg, 555 μmol) in DCM (4 mL) were added cat. acetic acid followed by 1,1-di(methyl)ethyl 3-(methylamino)azetidine-1-carboxylate (132) (150 mg, 832 μmol) at 0° C. and the reaction mixture was stirred for 30 min at 25° C. To this was added sodium triacetoxyborohydride (176 mg, 832 μmol) at 0° C. portion wise and stirring was continued for 16 h at 25° C. After completion of reaction (as judged by TLC & LC/MS), the volatiles were removed under reduced pressure. The crude thus obtained was purified by combi flash column chromatography (SiO₂; 4 g, 15% MeOH-DCM) to afford methyl 7-[5-chloranyl-2-[2-[6-[[1-[1,1-di(methyl)ethoxycarbonyl]azetidin-3-yl]-methyl-amino]-2-methyl-4-oxidanylidene-5,6,7,8-tetrahydroquinazolin-3-yl]ethoxy]phenyl]-5-methyl-thieno[3,2-b]pyridine-3-carboxylate (133) (0.220 g, 310.62 μmol, 60% yield) as a white solid.

MS calculated: 707; MS found: 708 (M+H).

### Synthesis of 134

To a stirred solution of methyl 7-[5-chloranyl-2-[2-[6-[[1-[1,1-di(methyl)ethoxycarbonyl]azetidin-3-yl]-methyl-amino]-2-methyl-4-oxidanylidene-5,6,7,8-tetrahydroquinazolin-3-yl]ethoxy]phenyl]-5-methyl-thieno[3,2-b]pyridine-3-carboxylate (133) (0.220 g, 310.62 μmol) in DCM (2 mL) was added HCl-4(M) in dioxane (2.0 mL. 8 mmol) at 0° C. and the reaction mixture was stirred at 25° C. for 2 h. After completion of reaction (as judged by LC/MS only), the reaction mixture was evaporated under reduced pressure. The crude thus obtained was purified by trituration with Et2O to give chlorane; methyl 7-[2-[2-[6-[azetidin-3-yl(methyl)amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (134) (150 mg, 232.70 μmol, 74.91% yield) as an off white solid, which was used to next step without further purification.

MS calculated: 607; MS found: 608 (M+H).

### Synthesis of 135: (Compound 66)

To a stirred solution of methyl 7-[2-[2-[6-[azetidin-3-yl(methyl)amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (134) (0.060 g, 98.66 μmol) in a mixture of THF (5 mL) and water (1 mL) was added LiOH·H2O (4.14 mg, 98.66 μmol, 2.74 μL) at 25° C. and the reaction mixture was stirred at 25° C. for 4 h. After completion of reaction (as judged by TLC and LCMS), the solvent was evaporated in vacuo. The crude thus obtained was purified by reverse phase prep-HPLC to get 7-[2-[2-[6-[azetidin-3-yl(methyl)amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylic acid; 2,2,2-tris(fluorenyl)acetic acid (21 mg, 29.43 μmol, 29.83% yield, 99.25% purity) (Compound 66) as a white solid.

1H NMR (400 MHz, DMSO-d6): 8.78 (s, 1H), 8.7-8.2 (m, 3H), 7.57-7.54 (m, 1H), 7.42 (d, J=2.4 Hz 1H), 7.38 (s, 1H), 7.31 (t, J=9.2 Hz 1H), 4.35-3.5 (m, 10H), 2.75 (s, 5H), 2.35 (s, 3H), 2.1 (s, 1H), 1.9 (bs, 1H), 1.7 (s, 3H), 1.6 (bs, 1H) ppm.

m/z calc. 593, found 594 (M+H).

### Synthesis of 137

To a stirred solution of the compounds methyl 7-[2-[2-[6-[azetidin-3-yl(methyl)amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate HCl (134) (0.210 g, 325.78 μmol)) in NMP (4.0 mL) were added K₂CO₃ (134.87 mg, 977.33 μmol) followed by 2,2,2-tris(fluorenyl)ethyl tris(fluorenyl)methanesulfonate (136) (90.74 mg, 390.93 μmol, 56.32 μL) at 25° C. and the reaction mixture was stirred at 60° C. for 16 h. After completion of reaction, the reaction mixture was cooled to 25° C. The reaction mixture was quenched with NH₄Cl, aqueous was extracted with EtOAc. The combined organic layer was washed with brine, dried over anhydrous Na₂SO₄, filtered and filtrate was evaporated under reduced pressure. The crude was thus obtained was purified by trituration with Et2O to give methyl 7-[5-chloranyl-2-[2-[2-methyl-6-[methyl-[1-[2,2,2-tris(fluorenyl)ethyl]azetidin-3-yl]amino]-4-oxidanylidene-5,6,7,8-tetrahydroquinazolin-3-yl]ethoxy]phenyl]-5-methyl-thieno[3,2-b]pyridine-3-carboxylate (137) (130 mg, 188.36 μmol, 57.82% yield) as a brown gum.

MS calculated: 689; MS found: 690 (M+H).

### Synthesis of 138 (Compound 64)

To a stirred solution of the compound methyl 7-[5-chloranyl-2-[2-[2-methyl-6-[methyl-[1-[2,2,2-tris(fluorenyl)ethyl]azetidin-3-yl]amino]-4-oxidanylidene-5,6,7,8-tetrahydroquinazolin-3-yl]ethoxy]phenyl]-5-methyl-thieno[3,2-b]pyridine-3-carboxylate (137) (0.100 g, 144.89 μmol) in a mixture of THF (5 mL) and water (1 mL) was added LiOH·H2O (6.08 mg, 144.89 μmol, 4.03 μL) at 25° C. The reaction mixture was stirred at 25° C. for 4 h. After completion of reaction (as judged by TLC and LCMS), the solvent was evaporated in vacuo. The crude thus obtained was purified by reverse phase prep-HPLC to get 7-[5-chloranyl-2-[2-[2-methyl-6-[methyl-[1-[2,2,2-tris(fluorenyl)ethyl]azetidin-3-yl]amino]-4-oxidanylidene-5,6,7,8-tetrahydroquinazolin-3-yl]ethoxy]phenyl]-5-methyl-thieno[3,2-b]pyridine-3-carboxylic acid; 2,2,2-tris(fluorenyl)acetic acid (Compound 64) (8 mg, 9.28 μmol, 6.40% yield, 91.63% purity) as a white solid.

1H NMR (400 MHz, DMSO-d6): 7.56 (d, J=8.8 Hz 1H), 7.41 (d, J=14.4 Hz 2H), 7.31 (d, J=8.8 Hz 1H), 7.16 (bs, 1H), 7.02 (bs, 1H), 6.90 (bs, 1H), 4.35 (bs, 2H), 4.12 (bs, 2H), 3.74 (t, 2H), 3.50 (bs, 2H), 3.27 (d, J=10 Hz 2H), 2.76 (s, 3H), 2.66 (s, 4H), 2.55 (s, 2H), 2.08-2.04 (m, 2H), 1.74 (s, 3H) ppm.

m/z calc. 675, found 674 (M−H).

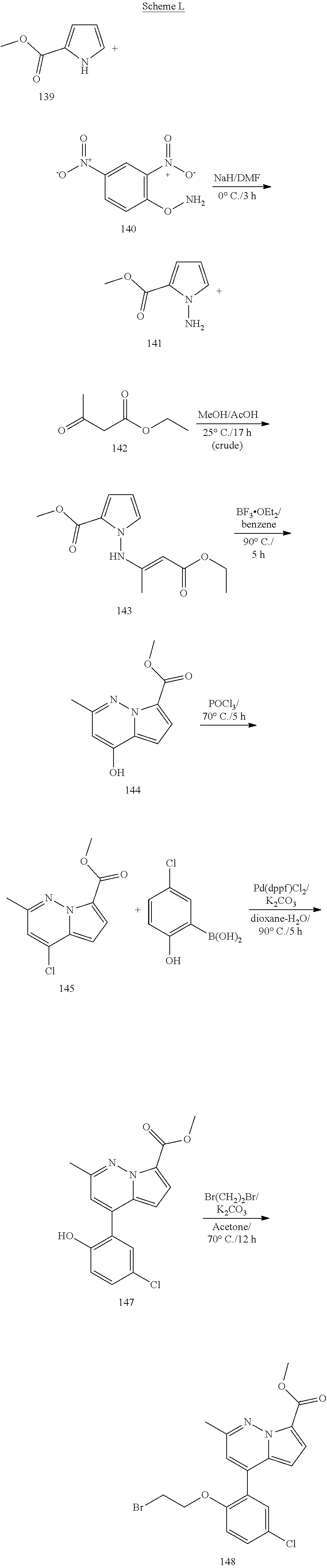

### Synthesis of 141

To a stirred solution of methyl 1H-pyrrole-2-carboxylate (139) (10 g, 79.92 mmol) in DMF (120 ml) was added sodium hydride (60%, 5.51 g, 239.76 mmol) at 0° C. in portion wise and the reaction mixture was stirred for 1 h at the same temperature. Then 0-(2,4-dinitrophenyl) hydroxylamine (140) (23.87 g, 119.88 mmol) in DMF (30 ml) was added drop wise at 0° C. and the reaction mixture was stirred for 3 h at the same temperature. After completion of reaction, the reaction mixture was diluted with saturated aqueous sodium thiosulfate solution (1000 ml), extracted with EtOAc (4×1000 ml). The combined organic layer was washed with brine (2×1000 ml), dried over anhydrous sodium sulphate, filtered and concentrated under reduced pressure to get crude. The crude residue was purified by combiflash column purification (SiO₂, 120 g, 5% EtOAc in Hexanes) to afforded methyl 1-azanylpyrrole-2-carboxylate (141) (11 g, 78.49 mmol, 98.21% yield) as a light-yellow sticky liquid.

1H NMR (400 MHz, DMSO-d6): 7.02-7.01 (m, 1H), 6.71-6.70 (m, 1H), 6.25 (s, 2H), 5.98-5.96 (m, 1H), 3.74 (s, 3H) ppm.

### Synthesis of 143

To a stirred solution of methyl 1-azanylpyrrole-2-carboxylate (141) (5 g, 35.68 mmol) in methanol (200 ml) in were added ethyl 3-oxidanylidenebutanoate (142) (5.57 g, 42.81 mmol, 5.44 mL) followed by acetic acid (50 ml) at 25° C. and the reaction mixture was stirred at 25° C. for 16 h. After completion of reaction, the volatiles were removed under reduced pressure and crude was diluted with aqueous saturated NaHCO₃ solution. The aqueous was extracted with EtOAc, the combined organic layer was washed with brine, dried over anhydrous sodium sulphate, filtered and concentrated under reduced pressure to obtain methyl 1-[[(E)-3-ethoxy-1-methyl-3-oxidanylidene-prop-1-enyl]amino]pyrrole-2-carboxylate (143) (8 g, crude) as a yellow oil, which was forwarded to next step without further purification.

MS calculated: 252.2; MS found: 253.2 (M+H).

### Synthesis of 144

To a stirred solution of methyl 1-[[(E)-3-ethoxy-1-methyl-3-oxidanylidene-prop-1-enyl]amino]pyrrole-2-carboxylate (143) (8 g, 31.71 mmol) in benzene (80 ml) was added boron trifluoride diethyl etherate (13.50 g, 95.14 mmol, 11.7 ml) slowly at 25° C. and the reaction mixture was stirred for 5 h at 90° C. The reaction mixture cooled to 25° C. and stirred it for another 17 h at the same temperature. After completion of reaction, the reaction mixture was quenched with water and extracted with ethyl acetate. The combined organic part was washed with brine and dried over anhydrous sodium sulphate, filtered and concentrated under reduced pressure. The crude which was purified in combiflash column chromatography (SiO₂; EtOAc:Hexanes 70%) to afford methyl 2-methyl-4-oxidanyl-pyrrolo[1,2-b]pyridazine-7-carboxylate (144) (2.5 g, 12.12 mmol, 38.23% yield) as an off white solid.

1H NMR (400 MHz, DMSO-d6): 11.72 (s, 1H), 7.28 (d, J=4.76 Hz, 1H), 6.61 (d, J=4.8 Hz, 1H), 6.19 (s, 1H), 3.78 (s, 3H), 2.39 (s, 3H) ppm.

MS calculated: 206; MS found: 207 (M+H).

### Synthesis of 145

To a mixture of phosphorus oxychloride (37.18 g, 242.49 mmol, 22.7 mL) and methyl 2-methyl-4-oxidanyl-pyrrolo[1,2-b]pyridazine-7-carboxylate (144) (2.5 g, 12.12 mmol) was heated at 70° C. for 5 h. After completion of reaction, the volatiles were removed under reduced pressure. The crude was purified in combiflash column chromatography (SiO₂; 40 g, 10% EtOAc:Hexane) to afford methyl 4-chloranyl-2-methyl-pyrrolo[1,2-b]pyridazine-7-carboxylate (145) (1.9 g, 8.46 mmol, 69.76% yield) as an off white solid.

1H NMR (400 MHz, DMSO-d6): 7.48 (d, J=4.6 Hz, 1H), 7.27 (s, 1H), 6.74 (d, J=4.6 Hz, 1H), 3.82 (s, 3H), 2.5 (s, 3H) ppm.

MS calculated: 224; MS found: 224.8 (M+H).

### Synthesis of 147

To a stirred solution mixture of methyl 4-chloranyl-2-methyl-pyrrolo[1,2-b]pyridazine-7-carboxylate (1.9 g, 8.46 mmol) (145) and (5-chloranyl-2-oxidanyl-phenyl) boronic acid (146) (1.75 g, 10.15 mmol) in water (2 ml) & dioxane (20 ml) was added potassium carbonate (4.09 g, 29.60 mmol) at 25° C., and the reaction mixture was degassed with argon for 30 min. To this was added Pd(dppf)Cl₂ (1.86 g, 2.54 mmol) and the reaction mixture was again degassed with argon for 10 min. The reaction mixture was stirred at 90° C. for 5 h. After completion of reaction, the reaction mixture was filtered through celite bed and washed with EtOAc (2×30 ml). The organic layer was dried with anhydrous sodium sulphate, filtered and concentrated under reduced pressure. The crude was purified by combiflash column chromatography (SiO₂; 12 g, 50% EtOAc/hexane as eluent) to afford methyl 4-(5-chloranyl-2-oxidanyl-phenyl)-2-methyl-pyrrolo[1,2-b]pyridazine-7-carboxylate (147) (2.1 g, 6.63 mmol, 78.39% yield) as a light yellow solid.

1H NMR (400 MHz, DMSO-d6): 10.14 (s, 1H), 7.41-7.37 (m, 3H), 6.95 (s, 1H), 6.32 (d, 1H, J=4.4), 3.82 (s, 3H), 2.53 (s, 3H) ppm.

MS calculated: 316; MS found: 315.2 (M−H).

### Synthesis of 148

To a stirred solution of methyl 4-(5-chloranyl-2-oxidanyl-phenyl)-2-methyl-pyrrolo[1,2-b]pyridazine-7-carboxylate (147) (5 g, 15.79 mmol) in acetone (50 ml) was added 1,2-dibromoethane (29.66 g, 157.86 mmol, 13.60 ml) followed by potassium carbonate (7.64 g, 55.25 mmol) at 25° C. under nitrogen then stirred at 70° C. for 12 h. After completion of reaction, the insoluble material was filtered through sintered funnel and the filtrate was concentrated under reduced pressure. The crude was purified by combiflash column chromatography (SiO₂; 40 g, 70% EtOAc/Hexane) to afford methyl 4-[2-(2-bromanylethoxy)-5-chloranyl-phenyl]-2-methyl-pyrrolo[1,2-b]pyridazine-7-carboxylate (148) (3.5 g, 8.18 mmol, 51.81% yield, 99% purity) as an off white solid.

1H NMR (400 MHz, DMSO-d6): 7.56-7.53 (m, 2H), 7.41 (d, 1H, J=4.68), 7.27 (d, 1H, J=8.52), 7.02 (s, 1H), 6.36 (d, 1H, J=4.4), 4.37-4.36 (m, 2H), 3.82 (s, 3H), 3.64-3.63 (m, 2H), 2.54 (s, 3H) ppm.

MS calculated: 422; MS found: 423.1 (M+H).

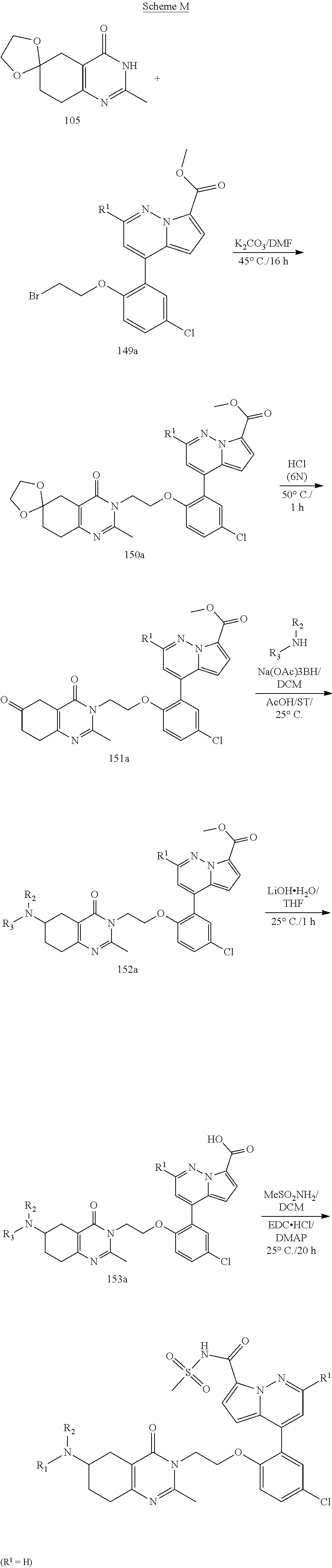

### Synthesis of 150a

To a stirred solution of methyl 4-[2-(2-bromanylethoxy)-5-chloranyl-phenyl]pyrrolo[1,2-b]pyridazine-7-carboxylate (149a) (737 mg, 1.80 mmol) in DMF (10 ml) were added 2′-methylspiro[1,3-dioxolane-2,6′-3,5,7,8-tetrahydroquinazoline]-4′-one (105) (400 mg, 1.80 mmol) followed by K₂CO₃ (746 mg, 5.40 mmol) at 25° C. under nitrogen atmosphere and the reaction mixture was stirred at 45° C. for 16 h. After completion of the reaction (as judged by LCMS and TLC), the reaction mixture was cooled to 25° C. and quenched with water. The aqueous phase was extracted with EtOAc (twice). The combined organic layer was washed with brine, dried over Na₂SO₄, filtered and the filtrate was evaporated under reduced pressure. The crude thus obtained was purified by combi flash column chromatography (SiO2; 12 g; 90% EtOAc/Hexanes) to give methyl 4-[5-chloranyl-2-[2-(2′-methyl-4′-oxidanylidene-spiro[1,3-dioxolane-2,6′-7,8-dihydro-5H-quinazoline]-3′-yl)ethoxy]phenyl]pyrrolo[1,2-b]pyridazine-7-carboxylate (150a) (410 mg, 744.12 μmol, 41.34% yield) as a yellow solid.

¹H NMR (400 MHz, DMSO-d6): 8.49 (d, J=4.44 Hz, 1H), 7.55-7.52 (m, 1H), 7.42 (d, J=2.52 Hz, 1H), 7.34 (d, J=4.64 Hz, 1H), 7.27 (d, J=8.52 Hz, 1H), 6.91 (d, J=4.64 Hz, 1H), 6.09 (d, J=4.68 Hz, 1H), 4.28-4.24 (m, 2H), 4.06-4.01 (m, 2H), 3.96 (s, 4H), 3.84 (s, 3H), 2.49-2.44 (m, 6H), 1.79 (t, J=6.36 Hz, 2H), 1.71 (s, 3H) ppm.

MS calculated: 550.16; MS found: 551.5 (M+H).

### Synthesis of 151a

To a stirred solution of methyl 4-[5-chloranyl-2-[2-(2′-methyl-4′-oxidanylidene-spiro[1,3-dioxolane-2,6′-7,8-dihydro-5H-quinazoline]-3′-yl)ethoxy]phenyl]pyrrolo[1,2-b]pyridazine-7-carboxylate (150a) (200 mg, 362.98 μmol) was added 6(N) HCl (2 ml) at 25° C. and the reaction mixture was stirred at 50° C. for 1 h. After completion of the reaction, the reaction mixture was quenched with aqueous NaHCO₃ solution. The aqueous phase was extracted with EtOAc (twice). The combined organic layer was washed with brine, dried over Na₂SO₄, filtered and the filtrate was evaporated under reduced pressure. The crude thus obtained was purified by combi flash column chromatography (SiO2, 4 g, 5% MeOH-DCM) to give methyl 4-[5-chloranyl-2-[2-[2-methyl-4,6-bis(oxidanylidene)-7,8-dihydro-5H-quinazolin-3-yl]ethoxy]phenyl]pyrrolo[1,2-b]pyridazine-7-carboxylate (151a) (116 mg, 228.83 μmol, 63.04% yield) as a yellow solid.

¹H NMR (400 MHz, DMSO-d6): 8.50 (d, J=4.56 Hz, 1H), 7.54 (dd, J=2.56 Hz, 1H), 7.42 (d, J=2.6 Hz, 1H), 7.35 (d, J=4.76 Hz, 1H), 7.28 (d, J=8.92 Hz, 1H), 6.91 (d, J=4.56 Hz, 1H), 6.08 (d, J=4.76 Hz, 1H), 4.29 (t, J=4.52 Hz, 2H), 4.10 (d, J=4.64 Hz, 2H), 3.84 (s, 3H), 3.09 (s, 2H), 2.79 (t, J=6.84 Hz, 2H), 2.56-2.53 (m, 2H), 1.76 (s, 3H) ppm.

MS calculated: 506.14; MS found: 507.4 (M+H).

### Synthesis of 152a

To a stirred solution of 4-[5-chloranyl-2-[2-[2-methyl-4,6-bis(oxidanylidene)-7,8-dihydro-5H-quinazolin-3-yl]ethoxy]phenyl]pyrrolo[1,2-b]pyridazine-7-carboxylate (151a) (80 mg, 157.81 μmol) in DCE (6 mL) were added cat. acetic acid followed by N-methylmethanamine (36 mg, 789.05 μmol, 45.90 μL) (2M solution 0.4 ml) at 0° C. and the reaction mixture was stirred for 2 h at 25° C. To this was added sodium triacetoxyborohydride (201 mg, 946.86 μmol) at 0° C. portion wise and stirring was continued for 2 h at 25° C. After completion of reaction (as judged by TLC & LC/MS), the volatiles were removed under reduced pressure. The crude was purified by combi flash column chromatography (amine functionalized; 4 g, 5% MeOH in DCM) to afford 4-[5-chloranyl-2-[2-[6-[di(methyl)amino]-2-methyl-4-oxidanylidene-5,6,7,8-tetrahydroquinazolin-3-yl]ethoxy]phenyl]pyrrolo[1,2-b]pyridazine-7-carboxylate (152a) (65 mg, 121.26 μmol, 76.84% yield) as light yellow solid.

¹H NMR (400 MHz, DMSO-d6): δ 8.50 (d, J=4.6 Hz, 1H), 7.56-7.53 (m, 1H), 7.42 (d, J=2.6 Hz, 1H), 7.35 (d, J=4.8 Hz, 1H), 7.28 (d, J=8.9 Hz, 1H), 6.91 (d, J=4.6 Hz, 1H), 6.91 (d, J=4.8 Hz, 1H), 4.29 (t, J=4.7 Hz, 2H), 4.07 (t, J=4.6 Hz, 2H), 3.84 (s, 3H), 2.23 (s, 6H), 2.17-2.13 (m, 1H), 1.92-1.89 (m, 1H), 1.73 (s, 3H), 1.53-1.50 (m, 1H), 1.23 (s, 4H) ppm.

MS calculated: 535.20; MS found: 536.0 (M+H).

### Synthesis of 153a: (Compound 30)

To a stirred solution of methyl 4-[5-chloranyl-2-[2-[6-[di(methyl)amino]-2-methyl-4-oxidanylidene-5,6,7,8-tetrahydroquinazolin-3-yl]ethoxy]phenyl]pyrrolo[1,2-b]pyridazine-7-carboxylate (152a) (65 mg, 121.26 μmol) in THF (1.2 ml), methanol (0.3 ml) & water (0.3 ml) was added lithium hydroxide monohydrate, 98% (15 mg, 363.79 μmol, 10.11 μL) at 25° C. and reaction mixture was stirred at 25° C. for 1 h. After completion of reaction, the volatiles were removed under reduced pressure and crude was purified by reverse phase preparative HPLC for purification to give 4-[5-chloranyl-2-[2-[6-[di(methyl)amino]-2-methyl-4-oxidanylidene-5,6,7,8-tetrahydroquinazolin-3-yl]ethoxy]phenyl]pyrrolo[1,2-b]pyridazine-7-carboxylic acid (Compound 30) (30 mg, 57.44 μmol, 47.37% yield, 99.94% purity) as a light green solid.

¹H NMR (400 MHz, DMSO-d6): δ 8.45 (d, J=4.5 Hz, 1H), 7.56-7.53 (m, 1H), 7.42 (d, J=2.7 Hz, 1H), 7.32-7.26 (m, 2H), 6.86 (d, J=4.5 Hz, 1H), 6.11 (d, J=4.8 Hz, 1H), 4.28 (t, J=4.9 Hz, 2H), 4.07 (t, J=4.9 Hz, 2H), 2.46 (s, 2H), 2.24 (s, 6H), 2.17-2.13 (m, 1H), 1.92-1.89 (m, 1H), 1.75 (s, 3H), 1.51 (brs, 1H) ppm.

MS calculated: 521.18; MS found: 522.2 (M+H).

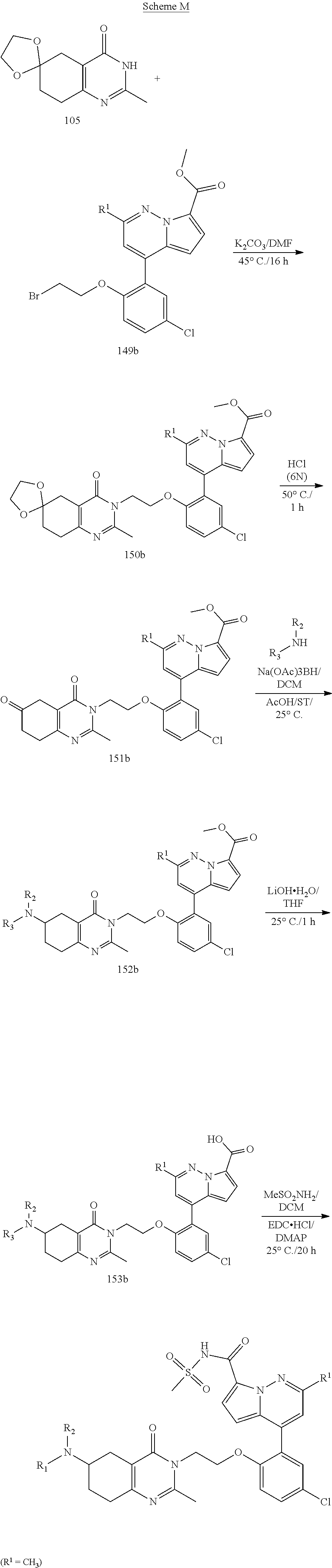

### Synthesis of 150b

To a stirred solution of 2′-methylspiro[1,3-dioxolane-2,6′-3,5,7,8-tetrahydroquinazoline]-4′-one (105) (200 mg, 899.93 μmol) in DMF (10 ml) were added potassium carbonate-granular (249 mg, 1.80 mmol) followed by methyl 4-[2-(2-bromanylethoxy)-5-chloranyl-phenyl]-2-methyl-pyrrolo[1,2-b]pyridazine-7-carboxylate (149b) (381 mg, 899.93 μmol) at 25° C. and the reaction mixture was stirred at 45° C. for 17 h. After completion of reaction, the reaction mixture was poured into cold water and extracted with EtOAc. The combined organic part was washed with brine, dried over anhydrous sodium sulphate, filtered and concentrated under reduced pressure. The crude was purified by combiflash column chromatography (SiO2, 12 g, 5% MeOH/DCM) to give methyl 4-[5-chloranyl-2-[2-(2′-methyl-4′-oxidanylidene-spiro[1,3-dioxolane-2,6′-7,8-dihydro-5H-quinazoline]-3′-yl) ethoxy]phenyl]-2-methyl-pyrrolo[1,2-b]pyridazine-7-carboxylate (150b) (250 mg, 442.47 μmol, 49.17% yield) as a white solid.

¹H NMR (400 MHz, DMSO-d6): δ 7.54-7.52 (m, 1H), 7.41 (d, J=2.5 Hz, 1H), 7.27-7.25 (m, 2H), 6.85 (s, 1H), 6.04 (d, J=4.5 Hz, 1H), 4.27 (s, 2H), 4.07 (s, 2H), 3.91 (s, 4H), 3.83 (s, 3H), 2.53 (s, 4H), 2.44 (s, 4H), 1.90-1.87 (m, 2H), 1.74 (s, 3H) ppm.

MS calculated: 564.18; MS found: 565.0 (M+H).

### Synthesis of 151b

A mixture of 4-[5-chloranyl-2-[2-(2′-methyl-4′-oxidanylidene-spiro[1,3-dioxolane-2,6′-7,8-dihydro-5H-quinazoline]-3′-yl) ethoxy]phenyl]-2-methyl-pyrrolo[1,2-b]pyridazine-7-carboxylate (150b) (560 mg, 991.12 μmol) and 6 (N) aqueous HCl (25 mL) was heated at 60° C. for 1.5 h. After completion of reaction, the reaction mixture was quenched with solid NaHCO₃ slowly under cooling condition (pH-8). The aqueous was extracted with EtOAc, the combined organic part was washed with brine, dried over anhydrous Na₂SO₄, filtered and concentrated under reduced pressure. The crude was purified by combiflash column chromatography (SiO2, 12 g, 2% MeOH/DCM) to give methyl 4-[5-chloranyl-2-[2-[2-methyl-4,6-bis(oxidanylidene)-7,8-dihydro-5H-quinazolin-3-yl]ethoxy]phenyl]-2-methyl-pyrrolo[1,2-b]pyridazine-7-carboxylate (151b) (405 mg, 777.41 μmol, 78.44% yield) as a yellow solid.

¹H NMR (400 MHz, DMSO-d6): δ 7.55-7.52 (m, 1H), 7.41 (d, J=2.6 Hz, 1H), 7.28-7.26 (m, 2H), 6.86 (s, 1H), 6.03 (d, J=4.8 Hz, 1H), 4.29 (t, J=4.5 Hz, 2H), 4.11 (t, J=4.6 Hz, 2H), 3.83 (s, 3H), 3.08 (s, 2H), 2.81-2.78 (m, 2H), 2.56-2.50 (m, 5H), 1.80 (s, 3H) ppm.

MS calculated: 520.15; MS found: 521.2 (M+H).

### Synthesis of 152b

To a stirred solution of methyl 4-[5-chloranyl-2-[2-[2-methyl-4,6-bis(oxidanylidene)-7,8-dihydro-5H-quinazolin-3-yl]ethoxy]phenyl]-2-methyl-pyrrolo[1,2-b]pyridazine-7-carboxylate (151b) (150 mg, 287.93 μmol) in 1,2-dichloroethane (8 mL) was added acetic acid (2 mg, 28.79 mol, 1.65 μL) followed by N,N-dimethylamine (65 mg, 1.44 mmol, 83.75 μL, 0.35 mL, 2 (M) solution in THF) at 25° C. and the reaction mixture was stirred at 25° C. for 2 h. To this was added sodium triacetoxyborohydride (305 mg, 1.44 mmol) at 0° C. and the reaction mixture was stirred at 25° C. for 2 h. The volatiles were removed under reduced pressure and the crude was purified by amine functionalized combiflash column chromatography (4 g, 0.2% methanol/DCM) to give methyl 4-[5-chloranyl-2-[2-[6-[di(methyl)amino]-2-methyl-4-oxidanylidene-5,6,7,8-tetrahydroquinazolin-3-yl]ethoxy]phenyl]-2-methyl-pyrrolo[1,2-b]pyridazine-7-carboxylate (152b) (130 mg, 236.34 μmol, 82.08% yield) as a light yellow solid.

1H NMR (400 MHz, DMSO-d6): δ 7.55-7.52 (m, 1H), 7.41 (d, J=2.4 Hz, 1H), 7.28-7.26 (m, 2H), 6.85 (s, 1H), 6.05 (d, J=4.7 Hz, 1H), 4.28-4.27 (m, 2H), 4.09-4.07 (m, 2H), 3.83 (s, 3H), 2.54 (s, 3H), 2.45-2.33 (m, 2H), 2.21 (s, 6H), 1.76 (s, 3H), 1.48-1.46 (m, 1H) ppm.

MS calculated: 549.21; MS found: 550.4 (M+H).

### Synthesis of 153b

To a stirred solution of methyl 4-[5-chloranyl-2-[2-[6-[di(methyl)amino]-2-methyl-4-oxidanylidene-5,6,7,8-tetrahydroquinazolin-3-yl]ethoxy]phenyl]-2-methyl-pyrrolo[1,2-b]pyridazine-7-carboxylate (13) (65 mg, 118.17 μmol) in a mixture of THF (1.2 ml), water (0.3 ml) & methanol (0.3 ml) was added lithium hydroxide, monohydrate (25 mg, 590.86 μmol) at 25° C. and the reaction mixture was stirred at 25° C. for 1 h. After completion of reaction, the volatiles were removed under reduced pressure and crude was purified by reverse phase preparative HPLC to give 4-[5-chloranyl-2-[2-[6-[di(methyl)amino]-2-methyl-4-oxidanylidene-5,6,7,8-tetrahydroquinazolin-3-yl]ethoxy]phenyl]-2-methyl-pyrrolo[1,2-b]pyridazine-7-carboxylic acid (153b) (26 mg, 48.19 μmol, 40.78% yield, 99.34% purity) as an off white solid.

1H NMR (400 MHz, DMSO-d6): δ 7.55-7.52 (m, 1H), 7.41 (d, J=2.7 Hz, 1H), 7.28-7.24 (m, 2H), 6.83 (s, 1H), 6.06 (d, J=4.8 Hz, 1H), 4.28 (t, J=4.8 Hz, 2H), 4.08 (t, J=4.8 Hz, 2H), 2.54 (s, 3H), 2.48-2.36 (m, 3H), 2.21 (s, 6H), 2.15-2.07 (m, 1H), 1.91-1.88 (m, 1H), 1.77 (s, 3H), 1.54-1.47 (m, 1H) ppm.

MS calculated: 535.20; MS found: 536.30 (M+H).

### Synthesis of Compound 59

To a stirred solution of 4-[5-chloranyl-2-[2-[6-[di(methyl)amino]-2-methyl-4-oxidanylidene-5,6,7,8-tetrahydroquinazolin-3-yl]ethoxy]phenyl]-2-methyl-pyrrolo[1,2-b]pyridazine-7-carboxylic acid (Compound 36) (60 mg, 111.94 μmol) in dichloromethane (1.5 ml) were added N,N-di(methyl)pyridin-4-amine (68 mg, 559.68 μmol) followed by chlorane; 3-(ethyliminomethyleneamino)-N,N-di(methyl)propan-1-amine (43 mg, 223.87 μmol) at 25° C. and the reaction mixture was stirred for 0.5 h at 25° C. To this was added methanesulfonamide (53.24 mg, 559.68 μmol) at 25° C. and the reaction mixture was stirred for 17 h at 25° C. After completion of reaction, the reaction mixture was diluted with DCM and washed with water, brine, dried over anhydrous Na₂SO₄, filtered and concentrated under reduced pressure. The crude was purified by reverse phase prep HPLC to give 4-[5-chloranyl-2-[2-[6-[di(methyl)amino]-2-methyl-4-oxidanylidene-5,6,7,8-tetrahydroquinazolin-3-yl]ethoxy]phenyl]-2-methyl-N-methylsulfonyl-pyrrolo[1,2-b]pyridazine-7-carboxamide (Compound 59) (29 mg, 45.96 μmol, 41.06% yield, 97.18% purity) as a light-yellow solid.

1H NMR (400 MHz, MeOD): δ 7.56-7.53 (m, 1H), 7.39 (d, J=2.3 Hz, 1H), 7.30-7.25 (m, 2H), 6.78 (s, 1H), 6.06 (d, J=4.7 Hz, 1H), 4.27-4.25 (m, 2H), 4.11 (s, 2H), 3.24 (s, 5H), 2.83 (s, 1H), 2.54 (s, 4H), 2.20-2.13 (m, 1H), 2.02-2.00 (m, 1H), 1.91 (s, 3H), 1.58-1.55 (m, 1H) ppm.

MS calculated: 612.19; MS found: 613.2 (M+H).

### Synthesis of 152c

To a stirred solution of methyl 4-[5-chloranyl-2-[2-[2-methyl-4,6-bis(oxidanylidene)-7,8-dihydro-5H-quinazolin-3-yl]ethoxy]phenyl]-2-methyl-pyrrolo[1,2-b]pyridazine-7-carboxylate (151b) (100 mg, 191.95 μmol) in 1,2-dichloroethane (3 ml) were added AcOH (6 mg, 95.98 mol, 5.49 μL) followed by piperidine (33 mg, 383.90 μmol, 37.92 l) at 25° C. and the reaction mixture was stirred for 5 h at that temperature. To this was added sodium triacetoxyborhydride (142 mg, 671.83 μmol) at 0° C. and the reaction mixture was stirred for 3 h at 25° C. After completion of reaction, the reaction mixture was concentrated under reduced pressure and crude thus obtained was purified by combi flash column chromatography (SiO₂; 12 g, 8% MeOH/DCM) to afford methyl 4-[5-chloranyl-2-[2-[2-methyl-4-oxidanylidene-6-(1-piperidyl)-5,6,7,8-tetrahydroquinazolin-3-yl]ethoxy]phenyl]-2-methyl-pyrrolo[1,2-b]pyridazine-7-carboxylate (152c) (90 mg, 152.51 μmol, 79.45% yield) as an yellow solid.

1H NMR (400 MHz, DMSO-d6): δ 7.54-7.52 (m, 1H), 7.44-7.41 (m, 1H), 7.28-7.26 (m, 2H), 6.84 (s, 1H), 6.04 (d, J=5 Hz, 1H), 4.28 (s, 2H), 4.07 (s, 2H), 3.83 (s, 3H), 2.41 (s, 6H), 1.75 (s, 3H), 1.49 (s, 4H), 1.39 (s, 3H) ppm.

MS calculated: 589.25; MS found: 590.1 (M+H).

### Synthesis of 153c (Compound 45)

To a stirred solution of 4-[5-chloranyl-2-[2-[2-methyl-4-oxidanylidene-6-(1-piperidyl)-5,6,7,8-tetrahydroquinazolin-3-yl]ethoxy]phenyl]-2-methyl-pyrrolo[1,2-b]pyridazine-7-carboxylate (152c) (90 mg, 152.51 μmol) in mixture of THF (2 ml):H2O (0.4 ml):MeOH (0.4 mL) was added LiOH·H2O (26 mg, 610.05 μmol) at 0° C. The reaction mixture was stirred for 1 h at 25° C. After completion of reaction, the reaction mixture was concentrated under reduced pressure and crude was purified by reverse phase Prep-HPLC to give 4-[5-chloranyl-2-[2-[2-methyl-4-oxidanylidene-6-(1-piperidyl)-5,6,7,8-tetrahydroquinazolin-3-yl]ethoxy]phenyl]-2-methyl-pyrrolo[1,2-b]pyridazine-7-carboxylic acid (153c) (Compound 45) (23 mg, 39.47 μmol, 25.88% yield, 98.87% purity) as a light yellow solid.

MS calculated: 575.23; MS found: 576.3 (M+H).

1H NMR (400 MHz, MeOD): δ 7.47-7.43 (m, 1H), 7.26-7.23 (m, 2H), 7.18 (d, J=2.3 Hz, 1H), 6.51 (s, 1H), 5.91 (d, J=4.5 Hz, 1H), 4.24 (s, 4H), 2.78-2.66 (m, 4H), 2.56 (s, 3H), 2.26-2.23 (m, 5H), 1.93-1.91 (m, 6H), 1.68 (s, 2H)ppm.

### Synthesis of Compound 62

To a stirred solution of 4-[5-chloranyl-2-[2-[2-methyl-4-oxidanylidene-6-(1-piperidyl)-5,6,7,8-tetrahydroquinazolin-3-yl]ethoxy]phenyl]-2-methyl-pyrrolo[1,2-b]pyridazine-7-carboxylic acid (Compound 45) (120 mg, 208.30 μmol) in dichloromethane (6 ml) were added DMAP (127 mg, 1.04 mmol) followed by EDC-HCl (79.86 mg, 416.61 μmol) and the reaction mixture was stirred for 0.5 h at 25° C. To this was added methanesulfonamide (99 mg, 1.04 mmol) at 25° C. and the reaction mixture was stirred for 16 h at 25° C. After completion of reaction, the reaction mixture was diluted with DCM and washed with water, brine, dried over anhydrous Na₂SO₄, filtered and the filtrate was concentrated under reduced pressure. The crude was purified by reverse phase prep HPLC to get 4-[5-chloranyl-2-[2-[2-methyl-4-oxidanylidene-6-(1-piperidyl)-5,6,7,8-tetrahydroquinazolin-3-yl]ethoxy]phenyl]-2-methyl-N-methylsulfonyl-pyrrolo[1,2-b]pyridazine-7-carboxamide (Compound 62) (24 mg, 36.46 μmol, 17.51% yield, 99.24% purity) as a light yellow solid.

MS calculated: 652.22; MS found: 653.3 (M+H).

1H NMR (400 MHz, DMSO): δ 7.56-7.53 (m, 1H), 7.39 (d, J=2.6 Hz, 1H), 7.30-7.25 (m, 2H), 6.79 (s, 1H), 6.04 (d, J=4.7 Hz, 1H), 4.31-4.25 (m, 2H), 4.11 (s, 2H), 3.25 (s, 4H), 2.81 (brs, 4H), 2.54 (s, 3H), 2.20-2.13 (m, 1H), 2.02-2.00 (m, 1H), 1.91 (s, 3H), 1.63 (s, 4H), 1.47 (s, 2H)ppm.

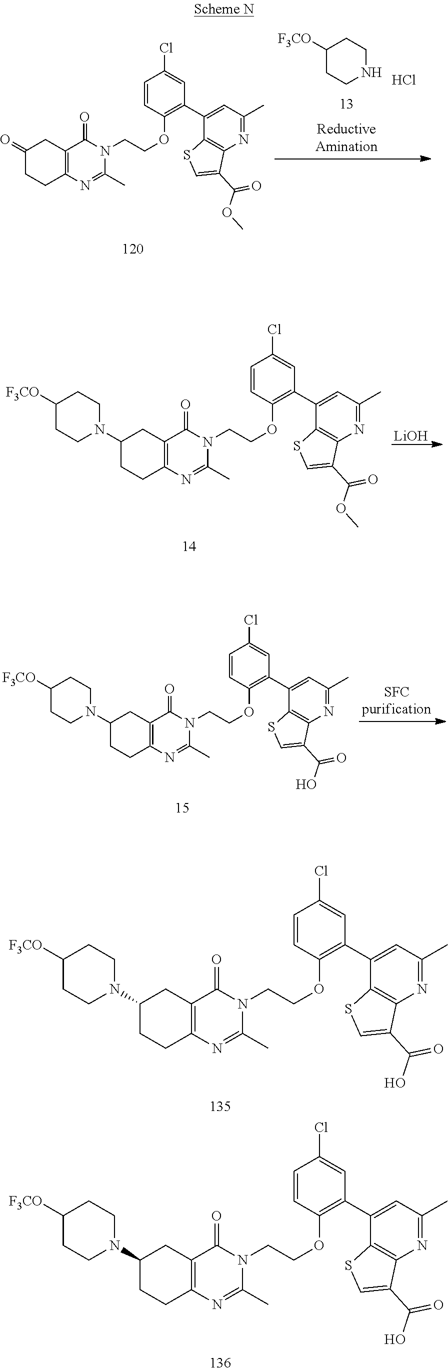

### Synthesis of Compound 135

To a stirred solution of 4-[tris(fluorenyl)methoxy]piperidine (18.86 g, 111.52 mmol, 2.0 eq) in dichloroethane (300 mL, 10 V) was added triethylamine (8.46 g, 83.64 mmol, 11.66 mL, 1.5 eq) at room temperature and stirred for 10 minutes, followed by addition of methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (30 g, 55.76 mmol, 1 eq) at same temperature and continued for 4 hours. After that, sodium cyano borohydride (3.50 g, 55.76 mmol 5.0 eq) was added to the above reaction mixture and it was continued at same temperature for 36 hours. Reaction was monitored by TLC and LCMS. After completion of the reaction, water (10 Vol.) was added to the reaction mass and extracted by 10% MeOH in DCM (10 Vol.). Collected organics, dried over sodium sulphate, and concentrated under reduced pressure to obtain crude. Crude compound was purified by reverse phase column chromatography (using celite, eluted at 45-50% Acetonitrile in 0.1% Ammonium Bicarbonate in water gradient) afforded methyl 7-(5-chloro-2-(2-(2-methyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylate (16 g, 41.57%) as a white solid. LCMS; 691.43 [M+1]

To a stirred solution of methyl 7-[5-chloranyl-2-[2-[2-methyl-4-oxidanylidene-6-[4-[tris(fluorenyl)methoxy]-1-piperidyl]-5,6,7,8-tetrahydroquinazolin-3-yl]ethoxy]phenyl]-5-methyl-thieno[3,2-b]pyridine-3-carboxylate (13 g, 18.81 mmol) in tetrahydrofuran (130 mL) and water (20 mL) was added lithium hydroxide (450.48 mg, 18.81 mmol) at 0° C. and resultant reaction was stirred at 25° C. for 2 hours. Reaction was monitored by TLC and LCMS. After completion, the reaction mixture was concentrated to remove the volatiles completely. The pH of the reaction mixture was adjusted to neutral with saturated citric acid and then extracted with 10% methanol in dichloromethane (3×200 mL). The combined organics was washed with water and brine, dried over sodium sulphate. Dried organics was filtrated and concentrated under reduced pressure to get racemic 7-(5-chloro-2-(2-(2-methyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylic acid (13.4 g, 85.48%) as a brown solid. The chiral isomers were separated by SFC to afford (S)-7-(5-chloro-2-(2-(2-methyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylic acid (Compound 135) as an off-white solid (4.5 g, 34.66%) and (R)-7-(5-chloro-2-(2-(2-methyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylic acid (Compound 136) (4.39 g, 33.81%) as an off white solid.

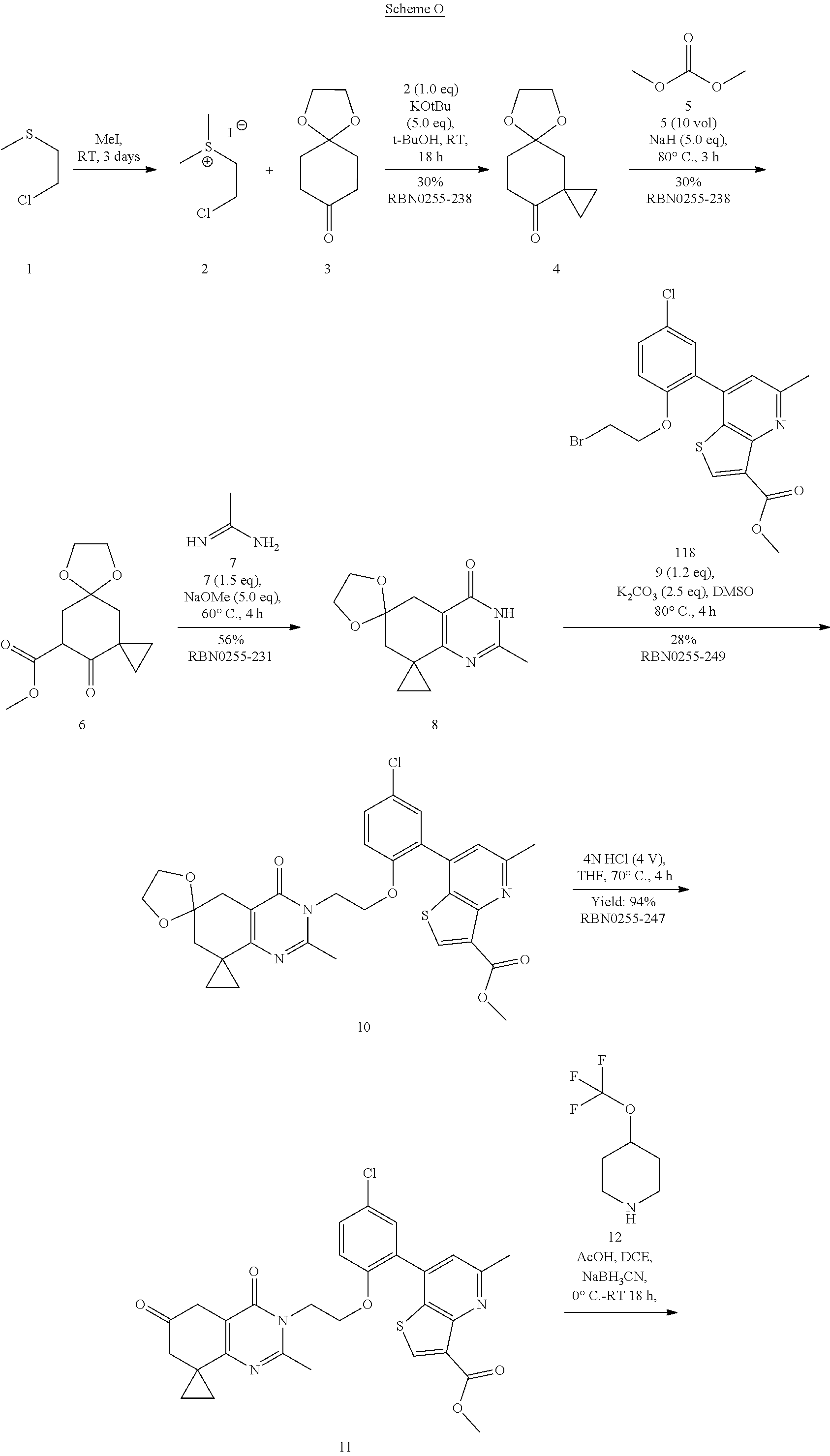

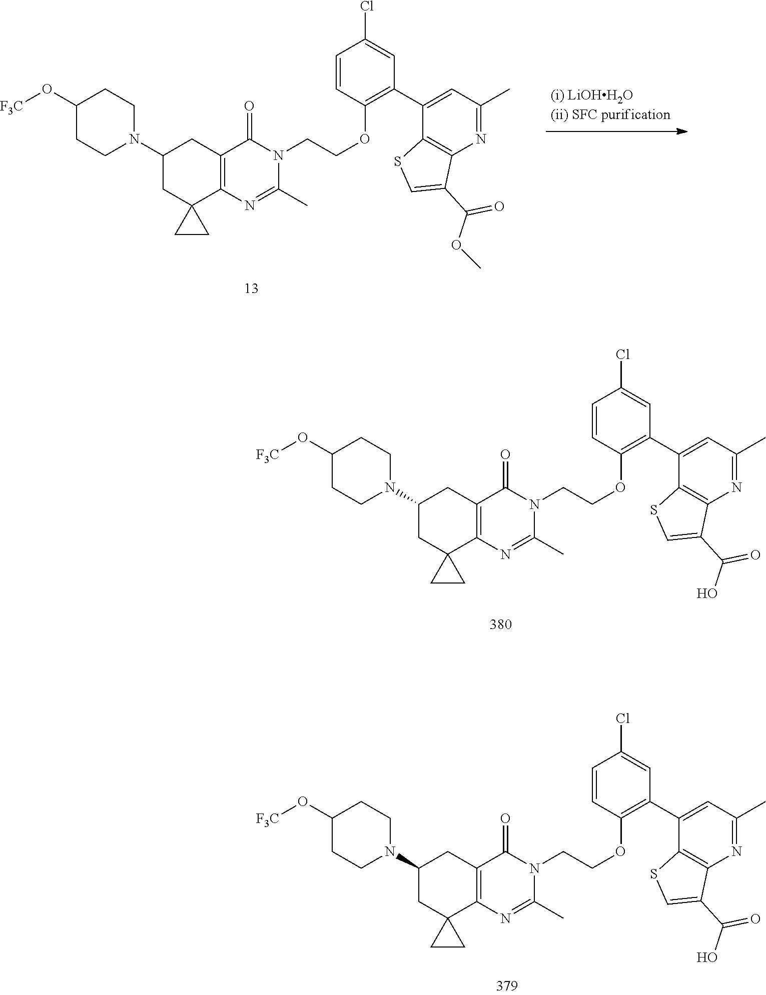 [SMILES: COC(=O)c1csc2c(-c3cc(Cl)ccc3OCCn3c(C)nc4c(c3=O)CC(N3CCC(OC(F)(F)F)CC3)CC43CC3)cc(C)nc12 ; Cc1cc(-c2cc(Cl)ccc2OCCn2c(C)nc3c(c2=O)C[C@@H](N2CCC(OC(F)(F)F)CC2)CC32CC2)c2scc(C(=O)O)c2n1 ; Cc1cc(-c2cc(Cl)ccc2OCCn2c(C)nc3c(c2=O)C[C@H](N2CCC(OC(F)(F)F)CC2)CC32CC2)c2scc(C(=O)O)c2n1]

### Synthesis of 2

To a stirred solution of 1-chloranyl-2-methylsulfanyl-ethane (25 g, 226.03 mmol) was added Mel (160.41 g, 1130 mmol) dropwise at 0° C. Reaction mixture was stirred at room temperature for 36 h. All volatiles were concentrated under reduced pressure to afford to crude compound. The crude compound was stirred in a diethyl ether solid formed was filtered through Buckner funnel and residue was washed with di ethyl ether and dried under reduced pressure to afford a required compound 2-chloroethyl-di(methyl) sulfonium iodide (2) as Brown solid (45 g, 78%).

1H NMR (DMSO-d6, 400 MHz): δ 4.16 (t, J=8.0 Hz, 2H), 3.01 (s, 6H).

### Synthesis of 4

To a stirred of solution of Potassium tertiary butoxide (9.34 g, 83.24 mmol) in t-BuOH (50 mL) was added 1,4-dioxaspiro[4.5]decan-8-one (10 g, 64.03 mmol). Reaction mixture stirred at room temperature for 15 min. to this (chloromethyl)dimethylsulfonium iodide (12.22 g, 51.22 mmol) was added portion wise at room temperature. Reaction mixture stirred at room temperature for 15 h. Reaction was monitored by the TLC. After completion of reaction, reaction mixture was quenched with water and extracted with ethyl acetate (2×200 mL) both organic layers were combined and washed with water, brine solution and dried over Na₂SO₄ and concentrated under reduced pressure and get crude compound. Crude compound was purified by combi flash column chromatography, eluted with 15-20% ethyl acetate in pet ether as pale-yellow oily liquid (3.5 g, 30%).

1H NMR (DMSO-d6, 400 MHz): δ 0.68 (t, J=4.0 Hz, 2H), 1.26 (t, J=4.0 Hz, 2H), 1.99 (s, 2H), 2.11 (t, J=8.0 Hz, 2H), 2.574 (t, J=8.0 Hz, 2H), 4.01 (m, 6H).

### Synthesis of 6

To a stirred solution 6,9-dioxadispiro[2.1.45.33]dodecan-12-one (1.2 g, 6.59 mmol) in Dimethyl carbonate (3 mL) was added sodium hydride 60% dispersion in mineral oil (757.01 mg, 32.93 mmol) in dimethyl carbonate (2 mL). Reaction mixture was stirred at room temperature for 5 min thereafter at 90° C. for 4 h. The reaction was monitored by the TLC and LCMS. After completion of reaction, reaction mixture was diluted with saturated solution of Ammonium chloride (20 mL), extracted with ethyl acetate (3×20 mL). The combined organic layer was thoroughly washed with saturated brine, dried over sodium sulphate and concentrated under reduced pressure to afford to crude compound as Brown oily liquid (1.2 g), which was used further without purification LCMS; [M+H]+: 241.1.

### Synthesis of 8

To a stirred solution of methyl 12-oxo-6,9-dioxadispiro[2.1.45.33]dodecane-11-carboxylate (1.2 g, 4.55 mmol) in sodium methoxide, 25% in methanol (2.46 g, 45.45 mmol, 2.53 mL), acetamidine hydrochloride, 97% (644.56 mg, 6.82 mmol) was added. Reaction mixture was stirred at 60° C. for 6 h. The reaction was monitored by TLC and LCMS. The reaction mixture acidified with 2N citric acid (10 mL) to pH (5-6) and extracted with ethyl acetate (3×20 mL). The combined organic layer was washed with water followed by brine solution, dried over sodium sulphate, filter and concentrated under reduced pressure to get crude compound. Crude compound was purified by column chromatography, eluted with MeOH:DCM (1-10%) to get a required compound as a pale yellow solid (500 g, 44.31%), LCMS; [M+H]+: 249.17

### Synthesis of 10

To a stirred solution of 8 in DMSO (5 mL) was added Potassium carbonate, anhydrous, 99% (949.83 mg, 6.87 mmol) followed by methyl 7-[2-(2-bromanylethoxy)-5-chloranyl-phenyl]-5-methyl-thieno[3,2-b]pyridine-3-carboxylate (1.594 g, 3.6 mmol) was added and reaction mixture was heated to 60° C. for 4 h. Reaction was monitored by the TLC and LCMS. After completion of reaction, reaction mixture was quenched with water (10 mL) and extracted with ethyl acetate (3×20 mL). The combined organic layer was washed with water followed by brine solution, dried over sodium sulphate, filter and concentrated under reduced pressure to get crude compound. Crude compound was purified by column chromatography, eluted with MeOH:DCM (1-5%) (650 mg, 38%), LCMS; [M+H]+: 608.48.

### Synthesis of 11

To a stirred solution of 10 (650 mg, 1.06 mmol) in THF (2 mL) was added 4M HCl (3 mL) at room temperature. Reaction mixture was stirred at 60° C. for 6 h. Reaction was monitored by the TLC and LCMS. After completion of reaction mixture was basified with saturated NaHCO₃ solution (10 ml) and extracted with Ethyl acetate (2×20 mL). The combined organic layer was washed with water, brine solution, dried over sodium sulphate and concentrated under reduced pressure to get crude material as yellow solid (450 mg, 30%), which was used further without purification LCMS; [M+H]+: 564.22.

### Synthesis of 13

To a solution of 12 (150. mg, 732.58 μmol) in DCE (1 mL), was added Triethylamine (4.40 mg, 43.48 μmol) at room temperature. Stirred the reaction mixture for 10 min followed by methyl 4-[5-chloranyl-2-[2-[2-methyl-4,6-bis(oxidanylidene)spiro[5,7-ihydroquinazoline-8,1′-cyclopropane]-3-yl]ethoxy]phenyl]-2-methyl-5H-cyclopenta[b]pyridine-7-carboxylate (200 mg, 366.29 μmol) was added and the resulting mixture was stirred for 16 h at room temperature. To this reaction mixture was added NaCNBH3 (1.45 g, 23.01 mmol) at room temperature and was stirred for 2 h at the same temperature. After completion of the reaction (monitored by TLC and LCMS), the reaction quenched with water (5 ml) and extracted with 10% MeOH:DCM (2×20 ml) then washed with brine. The combined organic layer was dried over sodium sulphate and concentrated to afford methyl 7-[5-chloranyl-2-[2-[2-methyl-4-oxidanylidene-6-[4-[tris(fluorenyl)methoxy]-1-piperidyl]spiro[6,7-dihydro-5H-quinazoline-8,1′-cyclopropane]-3-yl]ethoxy]phenyl]-5-methyl-thieno[3,2-b]pyridine-3-carboxylate as a grey solid. (200 mg, 25.68%); LCMS; [M+H]+: 717.80.

### Synthesis of Compound 379 and 380

To a solution methyl 7-[5-chloranyl-2-[2-[2-methyl-4-oxidanylidene-6-[4-[tris(fluorenyl)methoxy]-1-piperidyl]spiro[6,7-dihydro-5H-quinazoline-8,1′-cyclopropane]-3-yl]ethoxy]phenyl]-5-methyl-thieno[3,2-b]pyridine-3-carboxylate (200 mg, 278.86 μmol) in THE (0.7 mL) and water (0.3 ml), was added Lithium hydroxide monohydrate (29.26 mg, 697.16 mol) at room temperature. Resulting reaction mixture stirred for 4 h at room temperature. After completion of the reaction (monitored by TLC and LCMS), the reaction mixture was acidified (pH 5-6) with 2 N Citric acid and extracted with 10% MeOH:DCM (2×30 ml) then washed with brine. The combined organic layer was dried over sodium sulphate and concentrated to afford for 7-(5-chloro-2-(2-(2′-methyl-4′-oxo-6′-(4-(trifluoromethoxy)piperidin-1-yl)-6′,7′-dihydro-4′H-spiro[cyclopropane-1,8′-quinazolin]-3′(5′H)-yl)ethoxy)phenyl)-5-methylthieno[3,2-b]pyridine-3-carboxylic acid as racemic mixture. This was purified by prep and pure fraction was concentrated to afford the racemic and subjected to SFC purification for separation of enantiomers 379 and 380 (29 mg, 14.69%) LCMS: 717.80 [M+1](NMR data in table).

The following compounds in Table E1 are synthesized according to schemes above:

TABLE E1

| Compound No. | Schemes of intermediates | Final Step |
|---|---|---|
| 66 | A, D, E | Scheme K |
| 64 | A, D, E | Scheme K |
| 63 | A, D, E | Scheme I |
| 62 | A | Scheme M |
| 61 | A, D, E, F | Scheme H |
| 60 | A, D, E, F | Scheme G |
| 59 | A | Scheme M |
| 58 | A, D, E | Scheme F |
| 57 | A, D, E | Scheme I |
| 56 | A, D, E, F | Scheme H |
| 55 | A, D, E | Scheme I |
| 54 | A, D, E | Scheme F |
| 53 | A, D, E | Scheme F |
| 51 | A, D, E, F | Scheme H |
| 50 | A, D, E | Scheme F |
| 49 | A, D, E | Scheme F |
| 47 | A, D, E, F | Scheme J |
| 46 | A, D, E | Scheme F |
| 45 | A | Scheme M |
| 41 | A, D, E | Scheme I |
| 40 | A, D, E | Scheme I |
| 39 | A, D, E, F | Scheme H |
| 36 | A | Scheme M |
| 35 | A, D, E | Scheme F |
| 34 | A, D, E | Scheme F |
| 33 | A, D, E | Scheme F |
| 32 | A, D, E | Scheme F |
| 30 | A | Scheme M |
| 29 | A, D, E | Scheme F |
| 28 | A, D, E | Scheme F |
| 27 | A, D, E | Scheme F |
| 26 | A, D, E | Scheme F |
| 25 | A, D, E, F | Scheme G |
| 24 | A, D, E, F | Scheme G |
| 23 | A, D, E | Scheme F |
| 22 | A, D, E | Scheme F |
| 21 | A, D, E | Scheme F |
| 20 | A, D, E | Scheme F |
| 19 | A, D, E | Scheme F |
| 18 | A, D, E | Scheme F |
| 17 | A, D, E | Scheme F |
| 14 | A, D, E, F | Scheme G |
| 13 | A, D, E, F | Scheme G |
| 12 | A, D, E | Scheme F |
| 11 | A, D, E | Scheme F |
| 10 | A, D, E | Scheme F |
| 9 | A, D, E | Scheme F |
| 8 | A, D, E | Scheme F |
| 4 | A, D, E | Scheme F |
| 3 | A, D, E | Scheme F |
| 2 | A, B | Scheme C |
| 1 | A, B | Scheme C |

### Reductive Amination Reaction:

### Condition A1: (Titanium Isopropoxide/Room Temperature):

Titanium isopropoxide (5.0 eq.) was added to a mixture of amine (1.5 eq.) and triethylamine (1.5 eq.) in 1,2-dichloromethane (10 V) at room temperature. Keto compound (1.0 eq.) was added and resulting reaction mixture was stirred at room temperature for 16 h. It was then cooled to 0° C. and sodium cyanoborohydride (5.0 eq.) was added in portions. The resulting reaction mixture was stirred for 2 h. Progress of the reaction was monitored by TLC and LCMS. The reaction mixture was poured into ice-cold water and filtered through a celite bed followed by washes with 10% methanol in dichloromethane. The organic layer was separated, and the aqueous layer was washed again with 10% methanol in dichloromethane. The combined organic layer was concentrated under reduced pressure to obtain crude material sample. This was purified by flash column chromatography using 60-120 mesh silica gel and the desired product was eluted at 0-10% methanol in dichloromethane as the eluent. Pure fractions (determined by TLC) were combined and evaporated under reduced pressure to afford desired product.

### Condition A2: (Titanium Isopropoxide/80° C.):

Titanium isopropoxide (5.0 eq.) was added to a mixture of amine (1.5 eq.) and triethylamine (1.5 eq.) in 1,2-dichloromethane (10 V) at room temperature. Keto compound (1.0 eq.) was added and resulting reaction mixture was stirred at 80° C. for 16 h. It was then cooled to 0° C. and sodium cyanoborohydride (5.0 eq.) was added in portions. The resulting reaction mixture was stirred for 2 h. Progress of the reaction was monitored by TLC and LCMS. The reaction mixture was poured into ice-cold water and filtered through a celite bed followed by washes with 10% methanol in dichloromethane. The organic layer was separated, and the aqueous layer was washed again with 10% methanol in dichloromethane. The combined organic layer was concentrated under reduced pressure to obtain crude material sample. This was purified by flash column chromatography using 60-120 mesh silica gel and the desired product was eluted at 0-10% 10% methanol in dichloromethane as the eluent. Pure fractions (determined by TLC) were combined and evaporated under reduced pressure to afford desired product.

### Condition A3: (Titanium Isopropoxide/60° C.)

Titanium isopropoxide (5.0 eq.) was added to a mixture of amine (1.5 eq.) and triethylamine (1.5 eq.) in 1,2-dichloroethane (10 V) at room temperature. Keto compound (1.0 eq.) was added and resulting reaction mixture was stirred at 60° C. for 16 h. It was then cooled to 0° C. and sodium cyanoborohydride (5.0 eq.) was added in portions. The resulting reaction mixture was stirred for 2 h. Progress of the reaction was monitored by TLC and LCMS. The reaction mixture was poured into ice-cold water and filtered through a celite bed followed by washes with 10% methanol in dichloromethane. The organic layer was separated, and the aqueous layer was washed again with 10% methanol in dichloromethane. The combined organic layer was concentrated under reduced pressure to obtain crude material sample. This was purified by flash column chromatography using 60-120 mesh silica gel and the desired product was eluted at 0-10% 10% methanol in dichloromethane as the eluent. Pure fractions (determined by TLC) were combined and evaporated under reduced pressure to afford desired product.

### Condition A4: (Sodium Acetate/Toluene or Methanol/Room Temperature)

Sodium acetate (5.0 eq.) was added to a mixture of amine (1.5 eq.) and triethylamine (1.5 eq.) in toluene or methanol (10 V) at room temperature. Keto compound (1.0 eq.) was added and resulting reaction mixture was stirred at room temperature for 16 h. It was then cooled to 0° C. and sodium cyanoborohydride (5.0 eq.) was added in portions. The resulting reaction mixture was stirred for 2 h. Progress of the reaction was monitored by TLC and LCMS. The reaction mixture was poured into ice-cold water and filtered through a celite bed followed by washes with 10% methanol in dichloromethane. The organic layer was separated, and the aqueous layer was washed again with 10% methanol in dichloromethane. The combined organic layer was concentrated under reduced pressure to obtain crude material sample. This was purified by flash column chromatography using 60-120 mesh silica gel and the desired product was eluted at 0-10% methanol in dichloromethane as the eluent. Pure fractions (determined by TLC) were combined and evaporated under reduced pressure to afford desired product.

### Condition A5: (Sodium Acetate/60° C.):

Sodium acetate (5.0 eq.) was added to a mixture of amine (1.5 eq.) and triethylamine (1.5 eq.) in 1,2-dichloroethane (10 V) at room temperature. Keto compound (1.0 eq.) was added and resulting reaction mixture was stirred at 60° C. for 16 h. It was then cooled to 0° C. and sodium cyanoborohydride (5.0 eq.) was added in portions. The resulting reaction mixture was stirred for 2 h. Progress of the reaction was monitored by TLC and LCMS. The reaction mixture was poured into ice-cold water and filtered through a celite bed followed by washes with 10% methanol in dichloromethane. The organic layer was separated, and the aqueous layer was washed again with 10% methanol in dichloromethane. The combined organic layer was concentrated under reduced pressure to obtain crude material sample. This was purified by flash column chromatography using 60-120 mesh silica gel and the desired product was eluted at 0-10% methanol in dichloromethane as the eluent. Pure fractions (determined by TLC) were combined and evaporated under reduced pressure to afford desired product.

### Condition A6: (TEA/DCE or Methanol/Room Temperature):

Triethylamine (5.0 eq.) was added to a solution of amine (1.5 eq.) in 1,2-dichloroethane or methanol (10 V) at room temperature. Keto compound (1.0 eq.) was added and resulting reaction mixture was stirred at room temperature for 16 h. It was then cooled to 0° C. and sodium cyanoborohydride (5.0 eq.) was added in portions. The resulting reaction mixture was stirred for 2 h. Progress of the reaction was monitored by TLC and LCMS. The reaction mixture was poured into ice-cold water and filtered through a celite bed followed by washes with 10% methanol in dichloromethane. The organic layer was separated, and the aqueous layer was washed again with 10% methanol in dichloromethane. The combined organic layer was concentrated under reduced pressure to obtain crude material sample. This was purified by flash column chromatography using 60-120 mesh silica gel and the desired product was eluted at 0-10% methanol in dichloromethane as the eluent. Pure fractions (determined by TLC) were combined and evaporated under reduced pressure to afford desired product.

### Condition A7: (Titanium Isopropoxide/Methanol/80° C.)

Titanium isopropoxide (5.0 eq.) was added to a mixture of amine (1.5 eq.) and triethylamine (1.5 eq.) in methanol (10 V) at room temperature. Keto compound (1.0 eq.) was added and resulting reaction mixture was stirred at 60° C. for 16 h. It was then cooled to 0° C. and sodium cyanoborohydride (5.0 eq.) was added in portions. The resulting reaction mixture was stirred for 2 h. Progress of the reaction was monitored by TLC and LCMS. The reaction mixture was poured into ice-cold water and filtered through a celite bed followed by washes with 10% methanol in dichloromethane. The organic layer was separated, and the aqueous layer was washed again with 10% methanol in dichloromethane. The combined organic layer was concentrated under reduced pressure to obtain crude material sample. This was purified by flash column chromatography using 60-120 mesh silica gel and the desired product was eluted at 0-10% methanol in dichloromethane as the eluent. Pure fractions (determined by TLC) were combined and evaporated under reduced pressure to afford desired product.

### Condition A8: (Methanol/Acetic Acid):

Acetic acid (cat.) to a stirred solution of amine (1.0 eq.) and keto compound (1.1 eq.) in methanol (10 V) was added at room temperature and stirred for 5 h. After sodium cyanoborohydride (2 eq.) was added at 0° C., then allowed to room temperature and stirred for 16 h. Progress of reaction was monitored by TLC. After completion of the reaction, quenched with water and washed with 10% methanol in dichloromethane (50 mL×3). The combined organic layer was dried over anhydrous Na₂SO₄, filtered and the filtrate was evaporated under reduced pressure to obtain crude product. Crude product was purified by reverse phase flash chromatography (using gradient of 0-100% acetonitrile in 0.1% ammonium hydroxide in water, 230-400 mesh silica gel). The pure fraction was collected and concentrated under reduced pressure to afford desired product.

### Ester Hydrolysis (Using Lithium Hydroxide Monohydrate):

Lithium hydroxide monohydrate (5 eq.) was added to the solution of ester analogue (1 eq.) in a mixture of THF:H₂O (3:1) at room temperature. The resulting mixture was stirred at room temperature for xx h. Progress of the reaction was monitored by TLC and LCMS. The reaction mass was acidified with 1 N HCl/sat. citric acid solution to pH 4-6. The volatiles were removed under reduced pressure, washed 10-15% Methanol in Dichloromethane. The combined organic layer was concentrated under reduced pressure to obtain desired product.

### Boc Deprotection:

Boc derivative (1 eq.) dissolved in dichloromethane (10 V) followed by addition of 4.0 M hydrochloric acid in 1,4-dioxane (6 eq.) at 0° C. The reaction mixture was stirred at room temperature for another 2 h. Reaction was monitored by TLC. After completion of the reaction, excess solvent was evaporated under reduced pressure to obtain crude material. Crude material was further washed by diethyl ether (30 V). Obtained solid was further dried to furnish desired product.

### Alkylation Using 2,2,2-Tris(Fluorenyl)Ethyl Tris(Fluorenyl)Methanesulfonate:

Amine compound (1 eq.) in acetone (2 mL) and K₂CO₃ (7 eq.) was charged in a 25 mL three necked round bottom flask at room temperature. Resulting reaction mixture was stirred for 5 minutes and then 2,2,2-tris(fluorenyl)ethyl tris(fluorenyl)methanesulfonate (11 eq.) was added to above mixture. Resulting mixture was stirred at 80° C. for 4 h. After completion of the reaction, excess solvent was evaporated under reduced pressure to obtain crude material. Crude material was further purified by flash chromatography to furnish desired product.

### Alkylation of Quinazolin-4-One:

Potassium carbonate-granular (1 eq.) was added to a stirred solution quinazolin-4-one analogue (1 eq.) in DMSO (10 V) and the resulting mixture was stirred for 15 minutes at room temperature. Then, bromo compound (0.7 eq.) was added, then reaction mixture was heated at 75° C. for 3 h. Reaction was monitored by TLC. After completion of the reaction, ice cold water was added and formed precipitate was filtered. Obtained solid was washed with n-pentane to afford desired product.

### Sulfonamide Formation:

Condition F1: Methane sulfonamide (10 eq.), 4-dimethylaminopyridine (1.5 eq.) and EDC (3 eq.), DIPEA (3.5 eq.) were added to a stirred solution acid compound (1 eq.) in DCM (10 V) at 0° C. The resulting mixture was stirred at room temperature for 16 h.

Progress of the reaction was monitored by LC-MS. After completion of the reaction, reaction mixture was diluted with water and washed with DCM (2×100 mL), the combined organic layers were dried over anhydrous Na₂SO₄, filtered and concentrated to get crude product. The crude product was purified by prep-HPLC, the required pure fractions were collected and concentrated to desired product.

Condition F2: Methane sulfonamide (5eq.), CDI (3.5 eq.) and DBU (3.5 eq.) were added to a stirred solution acid compound (1 eq.) in DMF (10 V) at 0° C. The resulting mixture was stirred at 80° C. for 16 h. Progress of the reaction was monitored by LC-MS. After completion of the reaction, reaction mixture was diluted with water and washed with DCM (2×100 mL), the combined organic layers were dried over anhydrous Na₂SO₄, filtered and concentrated to get crude product. The crude product was purified by prep-HPLC, the required pure fractions were collected and concentrated to desired product.

Condition F3: Methane sulfonamide (10 eq.), HATU (3 eq.) & DIPEA (3.5 eq.) were added to a stirred solution acid compound (1 eq.) in THF (10 V) at 0° C. The resulting mixture was stirred at room temperature for 16 h. Progress of the reaction was monitored by LC-MS. After completion of the reaction, reaction mixture was diluted with water and washed with DCM (2×100 mL), the combined organic layers were dried over anhydrous Na₂SO₄, filtered and concentrated to get crude product. The crude product was purified by prep-HPLC, the required pure fractions were collected and concentrated to desired product.

### N-Methylation by Reductive Amination:

Formaldehyde, 37% solution and Sodium cyanoborohydride (337.75 mg, 5.37 mmol) were added to a stirred solution of amine (700 mg, 1.07 mmol) in methanol (10 V). After that, reaction mixture was stirred at room temperature for 16 h. Progress of the reaction was monitored by LC-MS. The reaction mixture was quenched with water and washed with 10% methanol in dichloromethane (2×100 mL) and the combined organic layers was dried over anhydrous Na₂SO₄, filtered and concentrated to get crude product. The crude product was purified by flash column chromatography by using 100-200 mesh silica gel eluting with 0-10% methanol in dichloromethane, the required pure fractions were combined and concentrated to desired product.

### Compound 532. (S)-7-(5-chloro-2-(2-(6-(5,6-difluoroisoindolin-2-yl)-2-methyl-4-oxo-5,6,7,8-tetrahydroquinazolin-3(4H)-yl)ethoxy)phenyl)-5-methylthieno[3,2-b]pyridine-3-carboxylic acid

### Compound 533. (R)-7-(5-chloro-2-(2-(6-(5,6-difluoroisoindolin-2-yl)-2-methyl-4-oxo-5,6,7,8-tetrahydroquinazolin-3(4H)-yl)ethoxy)phenyl)-5-methylthieno[3,2-b]pyridine-3-carboxylic

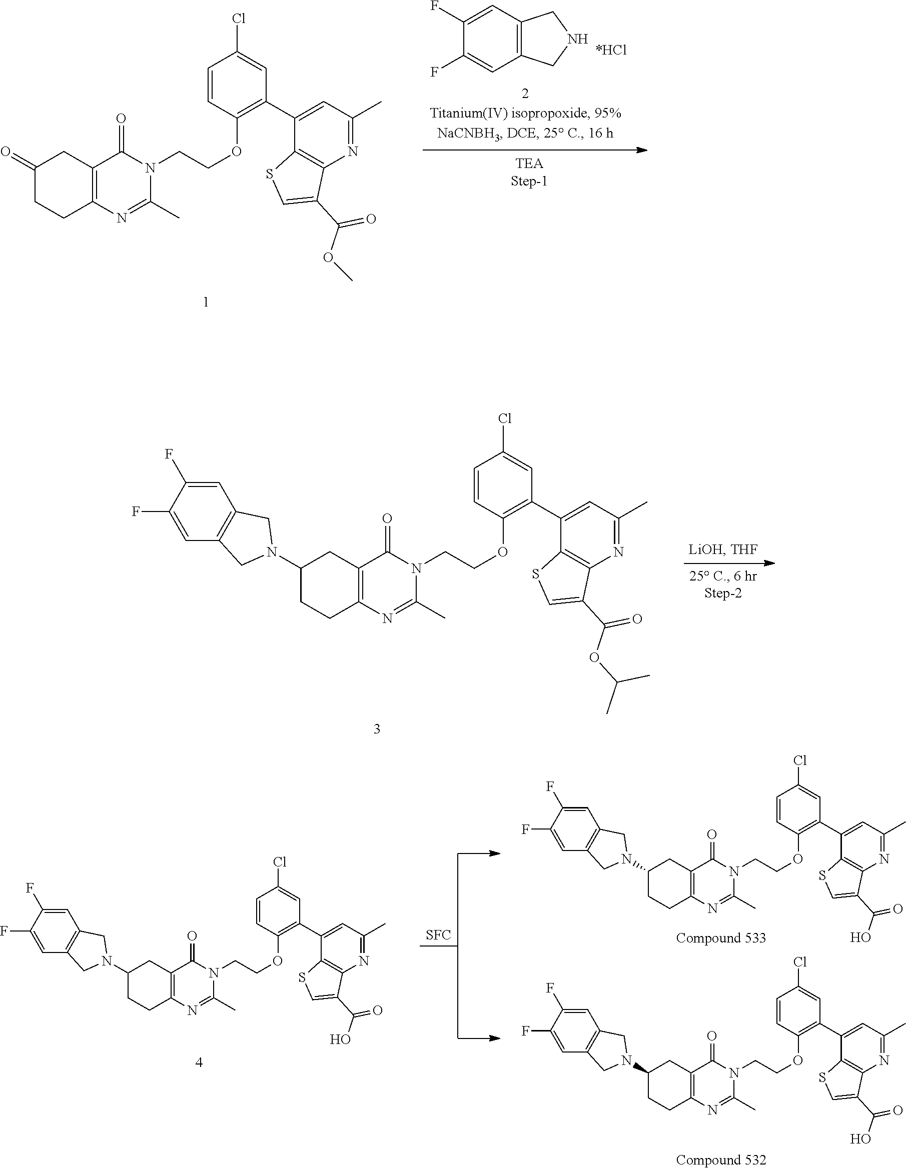

### Step-1: Synthesis of isopropyl 7-(5-chloro-2-(2-(6-(5,6-difluoroisoindolin-2-yl)-2-methyl-4-oxo-5,6,7,8-tetrahydroquinazolin-3(4H)-yl)ethoxy)phenyl)-5-methylthieno[3,2-b]pyridine-3-carboxylate (3)

Step 1 was synthesised using general procedure A2 starting from 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (350 mg, 650.54 μmol) and 5,6-bis(fluorenyl) isoindoline; hydrochloride (186.97 mg, 975.81 μmol) to afford isopropyl 7-(5-chloro-2-(2-(6-(5,6-difluoroisoindolin-2-yl)-2-methyl-4-oxo-5,6,7,8-tetrahydroquinazolin-3(4H)-yl)ethoxy)phenyl)-5-methylthieno[3,2-b]pyridine-3-carboxylate (400 mg, Crude material) as a light yellow solid. LCMS; 705.20 [M+1].

### Step-2: Synthesis of 7-(5-chloro-2-(2-(6-(5,6-difluoroisoindolin-2-yl)-2-methyl-4-oxo-5,6,7,8-tetrahydroquinazolin-3(4H)-yl)ethoxy)phenyl)-5-methylthieno[3,2-b]pyridine-3-carboxylic acid

Step 2 was synthesised using general procedure B starting from 1-methylethyl 7-[2-[2-[6-[5,6-bis(fluorenyl)isoindolin-2-yl]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (400 mg, 567.21 mol) and lithium hydroxide (67.92 mg, 2.84 mmol) to afford (S)-7-(5-chloro-2-(2-(6-(5,6-difluoroisoindolin-2-yl)-2-methyl-4-oxo-5,6,7,8-tetrahydroquinazolin-3(4H)-yl)ethoxy)phenyl)-5-methylthieno[3,2-b]pyridine-3-carboxylic acid and (R)-7-(5-chloro-2-(2-(6-(5,6-difluoroisoindolin-2-yl)-2-methyl-4-oxo-5,6,7,8-tetrahydroquinazolin-3(4H)-yl)ethoxy)phenyl)-5-methylthieno[3,2-b]pyridine-3-carboxylic acid. LCMS: 663.22 [M+1], 660.81 [M−1].

### 2. Experimental Procedure for 1-88 Examples: These were Prepared from Int-A (Big Keto) and the Appropriate Amine According to General Procedures Described for the Synthesis of Example 01

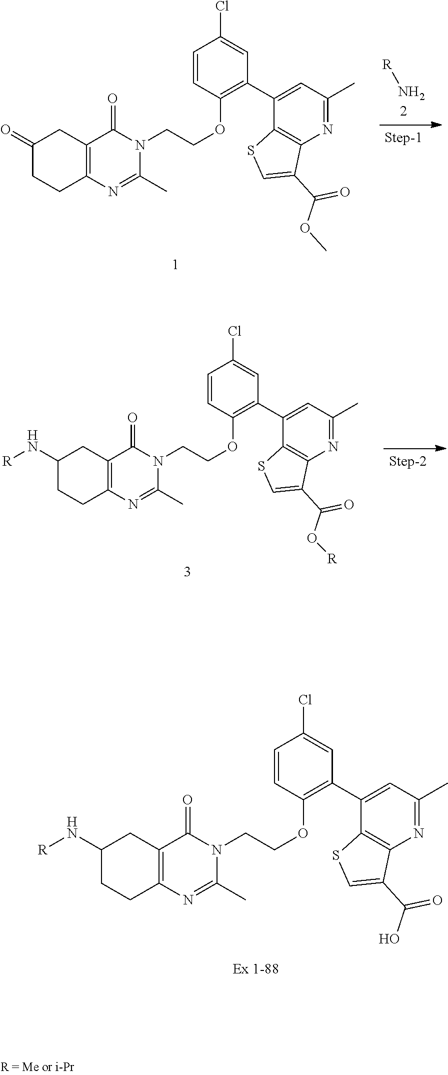

| | | | Method of | LCMS of 3: |
|---|---|---|---|---|
| Ex | Compound | Amines 2 | preparation | [M + H]⁺ |
| 1 | Compound 495 Compound 494 | 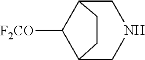 | A2 | 745.2 |
| 2 | Compound 482 Compound 481 Compound 484 Compound 483 | 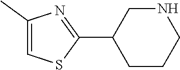 | A1 | 732.4 |
| 3 | Compound 490 Compound 489 | 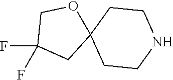 [SMILES: FC1(F)COC2(CCNCC2)C1] | A2 | 727.3 |
| 4 | Compound 578 Compound 577 | 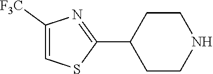 [SMILES: FC(F)(F)c1csc(C2CCNCC2)n1] | A3 | 786.2 |
| 5 | Compound 502 Compound 499 Compound 498 Compound 501 | 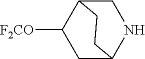 | A2 | 745.3 |
| 6 | Compound 435 Compound 434 | 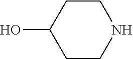 | A2 | 652.3 |
| 7 | Compound 406 Compound 405 Compound 404 Compound 403 | 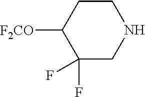 | A2 | 755.2 |
| 8 | Compound 563 Compound 562 Compound 552 Compound 551 | 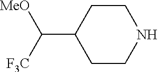 [SMILES: COC(C1CCNCC1)C(F)(F)F] | A1 | 748.2 |
| 9 | Compound 503 Compound 504 | 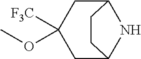 | A1 | 759.3 |
| 10 | Compound 594 Compound 593 | 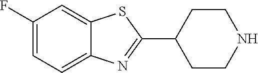 [SMILES: Fc1ccc2nc(C3CCNCC3)sc2c1] | A2 | 786.3 |
| 11 | Compound 111 Compound 110 | 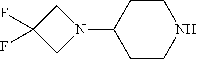 [SMILES: FC1(F)CN(C2CCNCC2)C1] | A7 | 697.2 |
| 12 | Compound 391 Compound 390 | 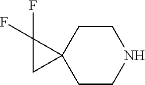 | A1 | 669.2 |
| 13 | Compound 445 Compound 444 | 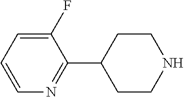 [SMILES: Fc1cccnc1C1CCNCC1] | A1 | 729.3 |
| 14 | Compound 429 Compound 428 | 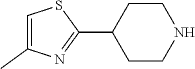 | A1 | 703.2 |
| 15 | Compound 480 Compound 479 Compound 478 Compound 477 | 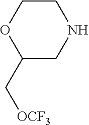 [SMILES: FC(F)(F)OCC1CNCCO1] | A1 | 706.2 |
| 16 | Compound 431 Compound 430 | 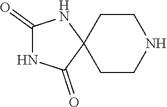 [SMILES: O=C1NC(=O)C2(CCNCC2)N1] | A2 | 720.2 |
| 17 | Compound 596 Compound 595 | 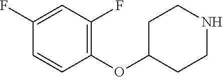 [SMILES: Fc1ccc(OC2CCNCC2)c(F)c1] | A2 | 763.3 |
| 18 | Compound 192 | 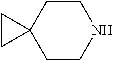 | A2 | 633.2 |
| 19 | Compound 526 Compound 525 Compound 524 Compound 523 | 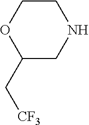 [SMILES: FC(F)(F)CC1CNCCO1] | A2 | 719.3 |
| 20 | Compound 214 Compound 215 | 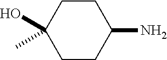 | A2 | 679.3 |
| 21 | Compound 407 Compound 452 | 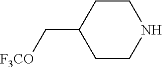 [SMILES: FC(F)(F)OCC1CCNCC1] | A2 | 733.4 |
| 22 | Compound 456 Compound 455 | 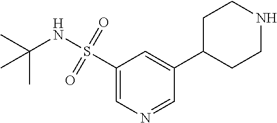 [SMILES: CC(C)(C)NS(=O)(=O)c1cncc(C2CCNCC2)c1] | A2 | 847.8 |
| 23 | Compound 454 Compound 453 | 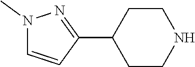 | A1 | 358.4 |
| 24 | Compound 156 | 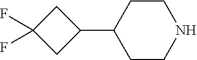 [SMILES: FC1(F)CC(C2CCNCC2)C1] | A2 | 697.2 |
| 25 | Compound 130 | 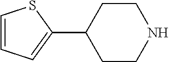 [SMILES: c1csc(C2CCNCC2)c1] | A3 | 692 |
| 26 | Compound 106 Compound 107 | 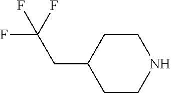 [SMILES: FC(F)(F)CC1CCNCC1] | A3 | 689.3 |
| 27 | Compound 384 Compound 383 Compound 382 Compound 381 | 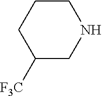 | A4 | 675.2 |
| 28 | Compound 109 Compound 108 | 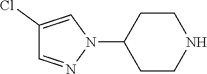 [SMILES: Clc1cnn(C2CCNCC2)c1] | A6 | 735.1 |
| 29 | Compound 378 Compound 377 Compound 376 Compound 415 | 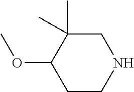 | A4 | 665.2 |
| 30 | Compound 451 Compound 450 | 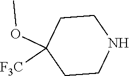 | A2 | 732.24 |
| 31 | Compound 585 Compound 584 | 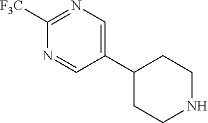 [SMILES: FC(F)(F)c1ncc(C2CCNCC2)cn1] | A1 | 781.40 |
| 32 | Compound 533 Compound 532 | 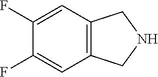 [SMILES: Fc1cc2c(cc1F)CNC2] | A2 | 353.94 |
| 33 | Compound 374 Compound 373 | 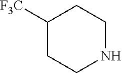 | A1 | 703.2 |
| 34 | Compound 576 Compound 575 | 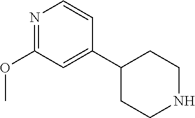 | A1 | 742.41 |
| 35 | Compound 574 Compound 573 | 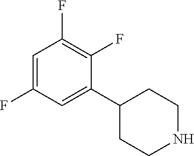 [SMILES: Fc1cc(F)c(F)c(C2CCNCC2)c1] | A1 | 765.39 |
| 36 | Compound 572 Compound 571 | 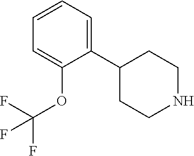 [SMILES: FC(F)(F)Oc1ccccc1C1CCNCC1] | A1 | 795.61 |
| 37 | Compound 586 | 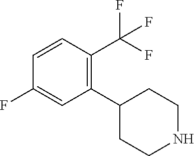 [SMILES: Fc1ccc(C(F)(F)F)c(C2CCNCC2)c1] | A1 | 797.31 |
| 38 | Compound 474 Compound 473 | 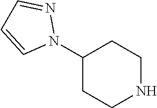 [SMILES: c1cnn(C2CCNCC2)c1] | A2 | 701.28 |
| 39 | Compound 461 Compound 460 | 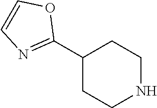 [SMILES: c1coc(C2CCNCC2)n1] | A2 | 702.60 |
| 40 | Compound 476 Compound 475 | 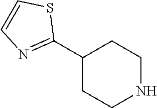 [SMILES: c1csc(C2CCNCC2)n1] | A2 | 718.78 |
| 41 | Compound 463 Compound 462 | 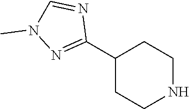 | A2 | 716.75 |
| 42 | Compound 174 | 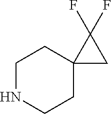 | A6 | 669.2 |
| 43 | Compound 209 | 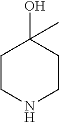 | A8 | 637.2 |
| 44 | Compound 157 | 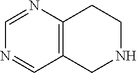 | A6 | 657.18 |
| 45 | Compound 154 | 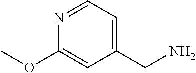 | A6 | 660.18 |
| 46 | Compound 165 | 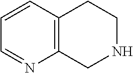 | A8 | 657.23 |
| 47 | Compound 143 | 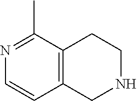 | A1 | 697.23 |
| 48 | Compound 427 Compound 426 | 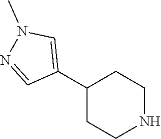 | A1 | 688.26 |
| 49 | Compound 425 Compound 424 | 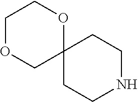 [SMILES: C1CC2(CCN1)COCCO2] | A1 | 705.23 |
| 50 | Compound 467 Compound 466 Compound 464 Compound 465 | 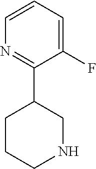 [SMILES: Fc1cccnc1C1CCCNC1] | A1 | 730.29 |
| 51 | Compound 448 Compound 447 Compound 446 | 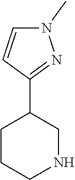 | A1 | 716.56 |
| 52 | Compound 472 Compound 458 Compound 457 Compound 471 | 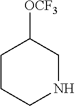 [SMILES: FC(F)(F)OC1CCCNC1] | A6 | 691.16 |
| 53 | Compound 485 | 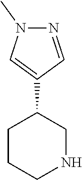 | A6 | 715.30 |
| 54 | Compound 487 Compound 486 | 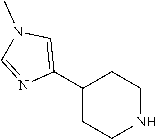 | A1 | 715.6 |
| 55 | Compound 180 | 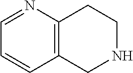 | A6 | 656.3 |
| 56 | Compound 212 | 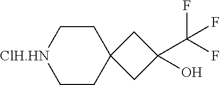 | A6 | 731.2 |
| 57 | Compound 166 Compound 167 Compound 168 Compound 169 | 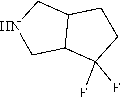 | A6 | 669.3 |
| 58 | Compound 120 Compound 113 Compound 121 Compound 122 | 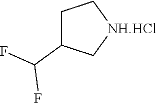 | A6 | 684.3 |
| 59 | Compound 389 Compound 388 Compound 387 Compound 386 | 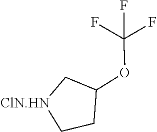 | A6 | 677.2 |
| 60 | Compound 152 | 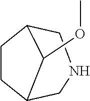 | A4 | 663.2 |
| 61 | Compound 146 Compound 147 Compound 148 Compound 149 | 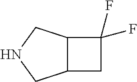 | A6 | 655.1 |
| 62 | Compound 128 Compound 131 | 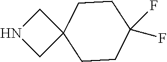 [SMILES: FC1(F)CCC2(CC1)CNC2] | A6 | 576.1 |
| 63 | Compound 423 Compound 422 | 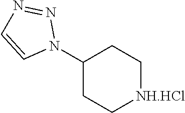 | A3 | 702.5 |
| 64 | Compound 421 Compound 420 | 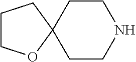 | A1 | 663.2 |
| 65 | Compound 134 | 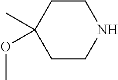 | A1 | 679.3 |
| 66 | Compound 126 | 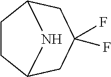 | A1 | 696.3 |
| 67 | Compound 402 Compound 401 | 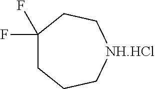 | A1 | 685.6 |
| 68 | Compound 360 Compound 385 | 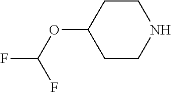 | A1 | 701.3 |
| 69 | Compound 124 | 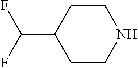 | A3 | 657.3 |
| 70 | Compound 115 | 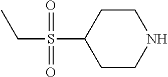 | A3 | 699.2 |
| 71 | Compound 419 Compound 418 | 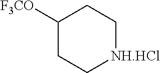 | A6 | 719.33 |
| 72 | Compound 470 Compound 469 | 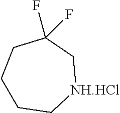 | A2 | 685.75 |
| 73 | Compound 164 | 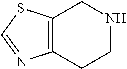 | A8 | 662.27 |
| 74 | Compound 163 | 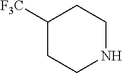 | A8 | 676.5 |
| 75 | Compound 170 | 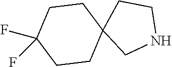 [SMILES: FC1(F)CCC2(CCNC2)CC1] | A6 | 698.23 |
| 76 | Compound 522 Compound 521 Compound 520 Compound 519 | 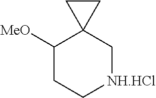 | A2 | 691.35 |
| 77 | Compound 132 | 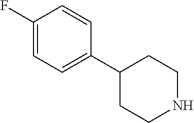 [SMILES: Fc1ccc(C2CCNCC2)cc1] | A6 | 703.2 |
| 78 | Compound 100 Compound 101 Compound 116 Compound 117 | 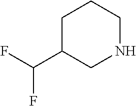 | A3 | 658.62 |
| 79 | Compound 409 Compound 437 Compound 436 Compound 408 | 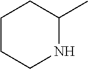 | A3 | 649.32 |
| 80 | Compound 449 Compound 488 | 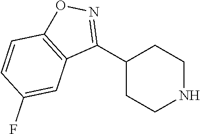 [SMILES: Fc1ccc2onc(C3CCNCC3)c2c1] | A3 | 771.72 |
| 81 | Compound 185 | 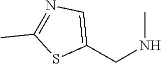 | A6 | 664.14 |
| 82 | Compound 171 | 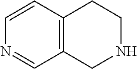 | A8 | 656.3 |
| 83 | Compound 119 | 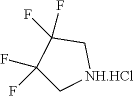 | A7 | 665.33 |
| 84 | Compound 367 Compound 366 | 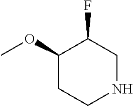 | A2 | 683.33 |
| 85 | Compound 413 Compound 412 | 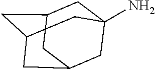 [SMILES: NC12CC3CC(CC(C3)C1)C2] | A2 | 673.2 |
| 86 | Compound 543 Compound 542 Compound 541 Compound 540 | 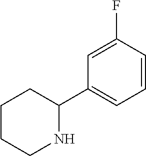 [SMILES: Fc1cccc(C2CCCCN2)c1] | A3 | 729.43 |
| 87 | Compound 549 Compound 548 Compound 547 Compound 546 | 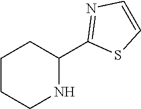 [SMILES: c1csc(C2CCCCN2)n1] | A3 | 718.33 |
| 88 | Compound 590 Compound 589 Compound 588 Compound 587 | 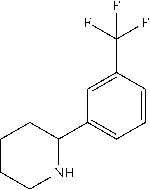 [SMILES: FC(F)(F)c1cccc(C2CCCCN2)c1] | A2 | 779.53 |

### 3. Experimental Procedure for Compound 512, Compound 511

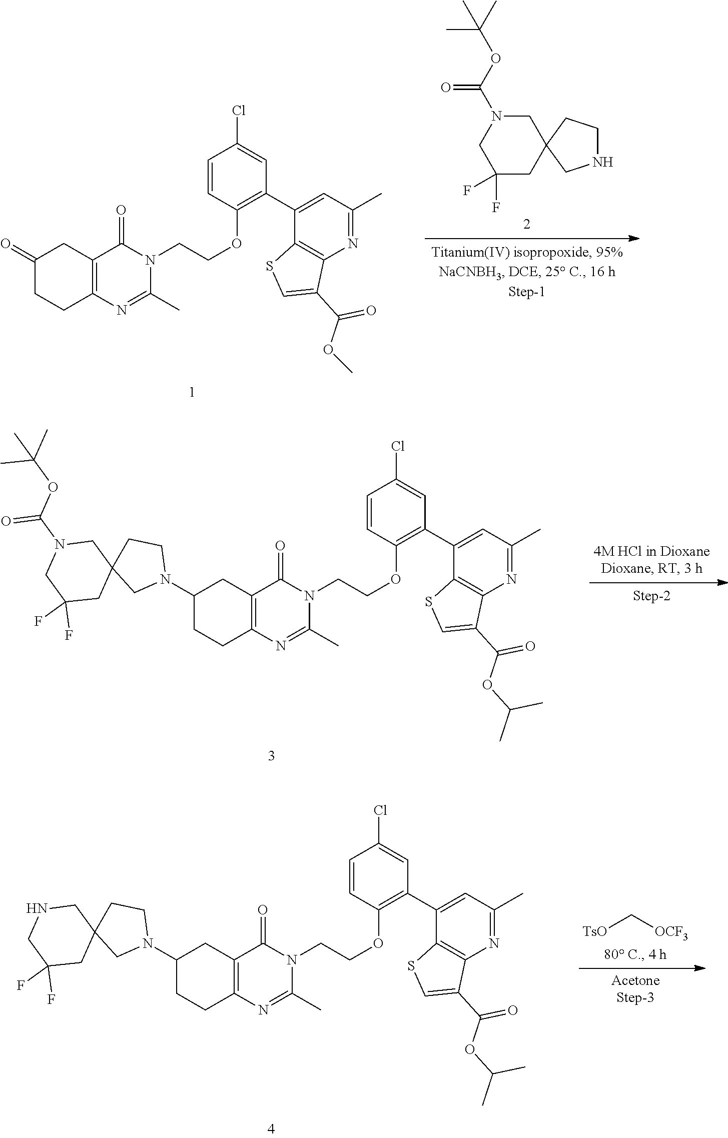 [SMILES: COC(=O)c1csc2c(-c3cc(Cl)ccc3OCCn3c(C)nc4c(c3=O)CC(=O)CC4)cc(C)nc12 ; CC(C)(C)OC(=O)N1CC(F)(F)CC2(CCNC2)C1 ; Cc1cc(-c2cc(Cl)ccc2OCCn2c(C)nc3c(c2=O)CC(N2CCC4(CN(C(=O)OC(C)(C)C)CC(F)(F)C4)C2)CC3)c2scc(C(=O)OC(C)C)c2n1 ; Cc1cc(-c2cc(Cl)ccc2OCCn2c(C)nc3c(c2=O)CC(N2CCC4(CNCC(F)(F)C4)C2)CC3)c2scc(C(=O)OC(C)C)c2n1 ; Cc1ccc(S(=O)(=O)OCOC(F)(F)F)cc1]

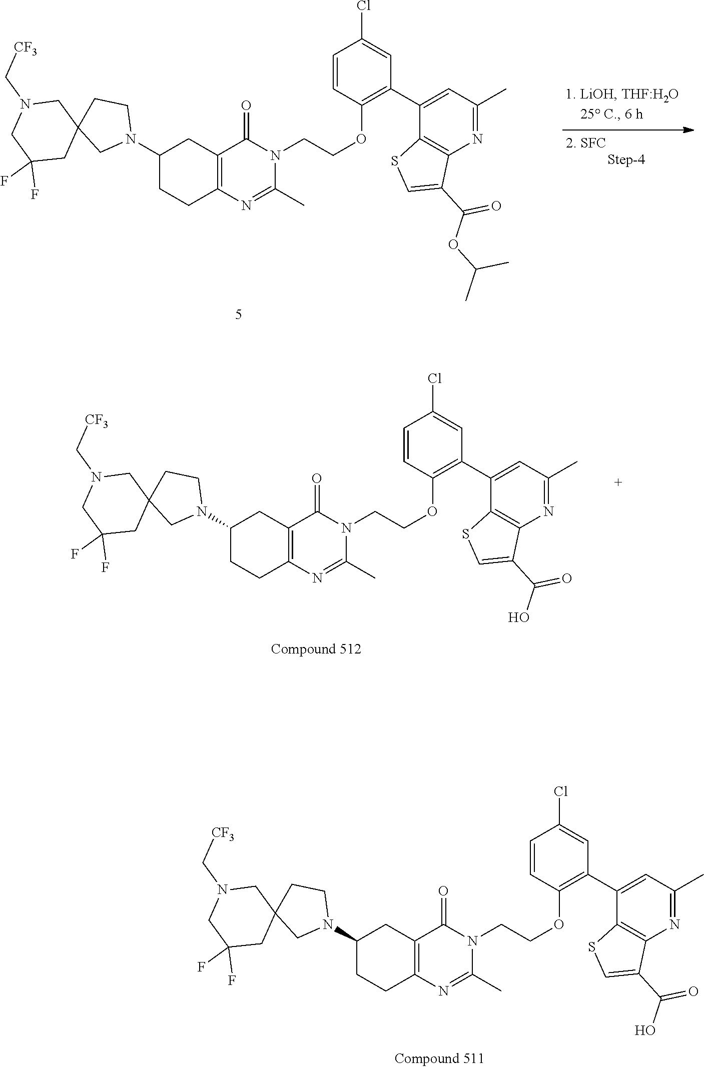 [SMILES: Cc1cc(-c2cc(Cl)ccc2OCCn2c(C)nc3c(c2=O)CC(N2CCC4(CN(CC(F)(F)F)CC(F)(F)C4)C2)CC3)c2scc(C(=O)OC(C)C)c2n1 ; Cc1cc(-c2cc(Cl)ccc2OCCn2c(C)nc3c(c2=O)C[C@@H](N2CCC4(CN(CC(F)(F)F)CC(F)(F)C4)C2)CC3)c2scc(C(=O)O)c2n1 ; Cc1cc(-c2cc(Cl)ccc2OCCn2c(C)nc3c(c2=O)C[C@H](N2CCC4(CN(CC(F)(F)F)CC(F)(F)C4)C2)CC3)c2scc(C(=O)O)c2n1]

### Step-1: Synthesis of methyl 7-(2-(2-(6-(7-(tert-butoxycarbonyl)-9,9-difluoro-2,7-diazaspiro[4.5]decan-2-yl)-2-methyl-4-oxo-5,6,7,8-tetrahydroquinazolin-3(4H)-yl)ethoxy)-5-chlorophenyl)-5-methylthieno[3,2-b]pyridine-3-carboxylate (3)

This compound was synthesised by common procedure A1 to afford methyl 7-(2-(2-(6-(7-(tert-butoxycarbonyl)-9,9-difluoro-2,7-diazaspiro[4.5]decan-2-yl)-2-methyl-4-oxo-5,6,7,8-tetrahydroquinazolin-3(4H)-yl)ethoxy)-5-chlorophenyl)-5-methylthieno[3,2-b]pyridine-3-carboxylate (400 mg, Crude material) as a light yellow solid. LCMS: 826.2 [M+1].

### Step-2: Synthesis of isopropyl 7-(5-chloro-2-(2-(6-(9,9-difluoro-2,7-diazaspiro[4.5]decan-2-yl)-2-methyl-4-oxo-5,6,7,8-tetrahydroquinazolin-3(4H)-yl)ethoxy)phenyl)-5-methylthieno[3,2-b]pyridine-3-carboxylate (4)

This compound was synthesised by common procedure C from methyl 7-(5-chloro-2-(2-(6-(9,9-difluoro-2,7-diazaspiro[4.5]decan-2-yl)-2-methyl-4-oxo-5,6,7,8-tetrahydroquinazolin-3(4H)-yl)ethoxy)phenyl)-5-methylthieno[3,2-b]pyridine-3-carboxylate to get isopropyl 7-(5-chloro-2-(2-(6-(9,9-difluoro-2,7-diazaspiro[4.5]decan-2-yl)-2-methyl-4-oxo-5,6,7,8-tetrahydroquinazolin-3(4H)-yl)ethoxy)phenyl)-5-methylthieno[3,2-b]pyridine-3-carboxylate as a light yellow solid. LCMS: 726.36 [M+1].

### Step 3: Synthesis of isopropyl 7-(5-chloro-2-(2-(6-(9,9-difluoro-2,7-diazaspiro[4.5]decan-2-yl)-2-methyl-4-oxo-5,6,7,8-tetrahydroquinazolin-3(4H)-yl)ethoxy)phenyl)-5-methylthieno[3,2-b]pyridine-3-carboxylate (5)

This compound was synthesised using common procedure D from 1-methylethyl 7-[2-[2-[6-[7,7-bis(fluorenyl)-2,9-diazaspiro[4.5]decan-2-yl]-2-methyl-4-oxidanylidene-5,6,7,8-tetrahydroquinazolin-3-yl]ethoxy]-5-chloranyl-phenyl]-5-methyl-benzothiophene-3-carboxylate (180 mg, 248.18 μmol) to afford isopropyl 7-(5-chloro-2-(2-(6-(9,9-difluoro-2,7-diazaspiro[4.5]decan-2-yl)-2-methyl-4-oxo-5,6,7,8-tetrahydroquinazolin-3(4H)-yl)ethoxy)phenyl)-5-methylthieno[3,2-b]pyridine-3-carboxylate.

### Step-4: Synthesis of 7-(5-chloro-2-(2-((6S)-6-(9,9-difluoro-7-(2,2,2-trifluoroethyl)-2,7-diazaspiro[4.5]decan-2-yl)-2-methyl-4-oxo-5,6,7,8-tetrahydroquinazolin-3(4H)-yl)ethoxy)phenyl)-5-methylthieno[3,2-b]pyridine-3-carboxylic acid (Compound 512)

This compound was synthesised by common procedure B using methyl 7-[2-[2-[6-[9,9-bis(fluorenyl)-7-[2,2,2-tris(fluorenyl)ethyl]-2,7-diazaspiro[4.5]decan-2-yl]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (200 mg, 256.33 μmol) to afford 7-[5-chloranyl-2-[2-[(6S)-6-[(7R)-9,9-difluoro-7-(2,2,2-trifluoroethyl)-2,7-diazaspiro[4.5]decan-2-yl]-2-methyl-4-oxidanylidene-5,6,7,8-tetrahydroquinazolin-3-yl]ethoxy]phenyl]-5-methyl-thieno[3,2-b]pyridine-3-carboxylic acid (6 mg, 7.54 μmol, 2.94% yield, 96.29% purity) and 7-[5-chloranyl-2-[2-[(6R)-6-[(7S)-9,9-difluoro-7-(2,2,2-trifluoroethyl)-2,7-diazaspiro[4.5]decan-2-yl]-2-methyl-4-oxidanylidene-5,6,7,8-tetrahydroquinazolin-3-yl]ethoxy]phenyl]-5-methyl-thieno[3,2-b]pyridine-3-carboxylic acid (9 mg, 11.46 μmol, 4.47% yield, 97.57% purity). LCMS: 766.32 [M+1].

The following ester derivatives are prepared using above protocol. Respective esters are further hydrolysed to final compounds.

| | | | Ester 5 LCMS |
|---|---|---|---|
| Ex | Compound | Amine | [M + H]⁺ |
| 1 | Compound 506 Compound 505 | 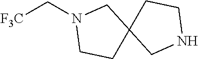 [SMILES: FC(F)(F)CN1CCC2(CCNC2)C1] | 789.2 |
| 2 | Compound 508 Compound 507 | 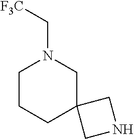 [SMILES: FC(F)(F)CN1CCCC2(CNC2)C1] | 758.3 |
| 3 | Compound 514 Compound 513 | 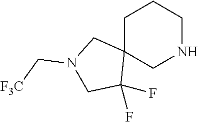 [SMILES: FC(F)(F)CN1CC(F)(F)C2(CCCNC2)C1] | 808.1 |
| 4 | Compound 518 Compound 517 | 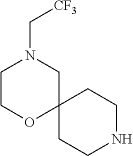 [SMILES: FC(F)(F)CN1CCOC2(CCNCC2)C1] | 788.2 |

### 4. Experimental Procedure for Compound 496 and Compound 497

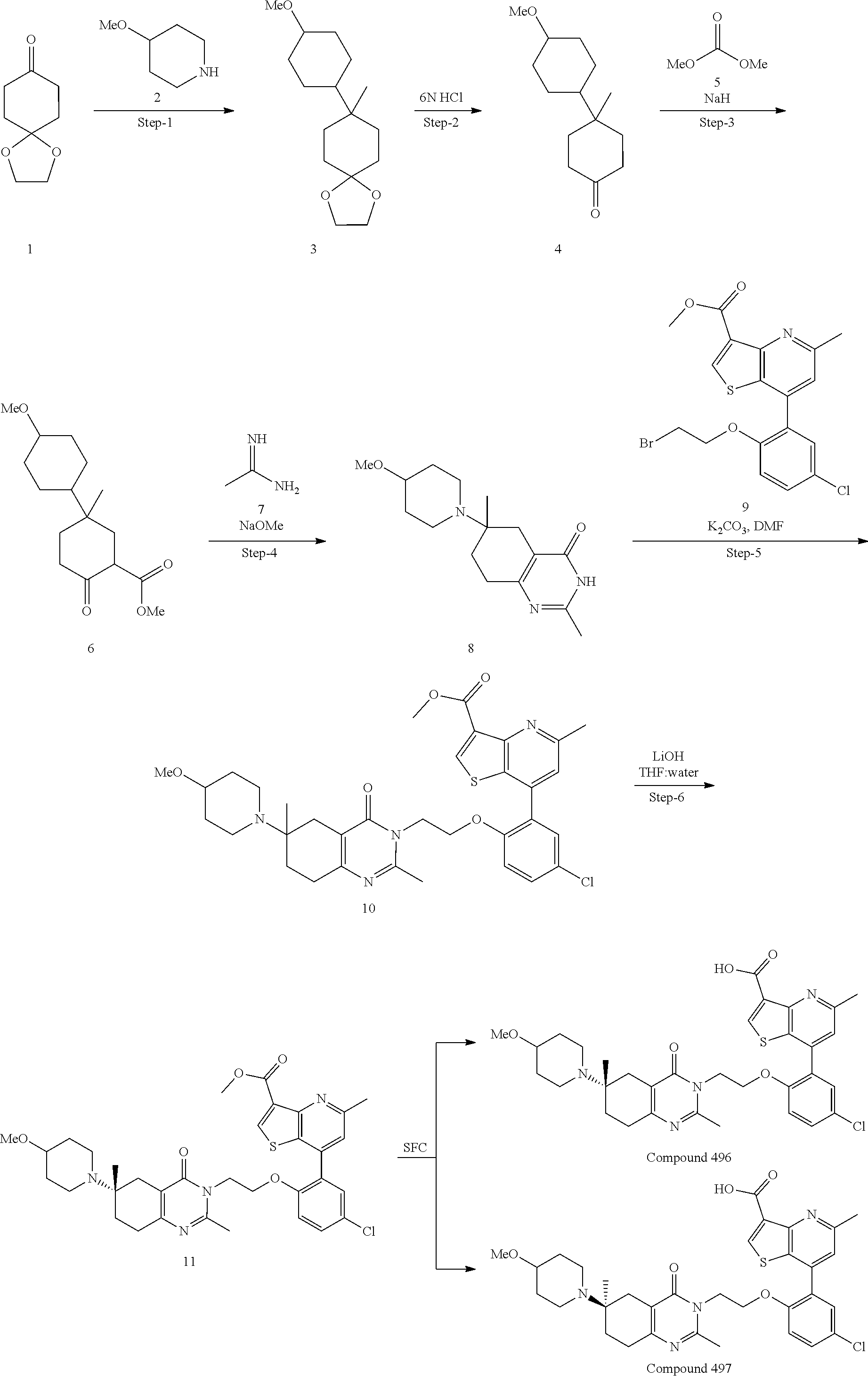

### Step-1: Synthesis of 4-methoxy-1-(8-methyl-1,4-dioxaspiro[4.5]decan-8-yl)piperidine (3)

A three-necked flask, equipped with a mechanical stirrer, a Dean-Stark trap, a condenser with nitrogen inlet-outlet was charged with 4-methoxypiperidine (5 g, 43.41 mmol), 1,4-dioxaspiro[4.5]decan-8-one (6.78 g, 43.41 mmol), 1,2,3-triazole (2.21 g, 32.01 mmol) and 50 mL of toluene. The reaction mixture was heated to 108-114° C. to achieve reflux and stirred for 6-8 h while collecting water via a Dean-Stark trap. The reaction mixture was cooled to room temperature and added to methyl magnesium bromide (200 mmol, 3.0 M in THF) over a period of 30 min with efficient stirring while maintaining the internal temperature at <24° C. The reaction mixture was stirred at room temperature for an additional 1 h. After completion, the reaction mixture was added to a 20% ammonium chloride solution over a period of 30 minutes while maintaining the internal temperature at <30° C. The organic layer was separated. The aqueous layer was washed with ethyl acetate (200 mL). The combined organic layers were washed with water (100 mL), and concentrated. The crude material was purified by silica gel chromatography to afford 4-methoxy-1-(8-methyl-1,4-dioxaspiro[4.5]decan-8-yl)piperidine as colourless oil (2.5 g, 22% yield). LCMS; 270.21 [M+1].

### Step-2: Synthesis of 4-(4-methoxy-1-piperidyl)-4-methyl-cyclohexanone (4)

To a stirred solution of 4-methoxy-1-(8-methyl-1,4-dioxaspiro[4.5]decan-8-yl)piperidine (2.0 g, 7.42 mmol) in tetrahydrofuran (20 mL) was added 6M HCl (20 mL) at 25° C. under nitrogen atmosphere. The reaction mixture was stirred at room temperature for 6 h. The progress of the reaction was monitored by LCMS. After completion, all volatiles were removed from reaction mixture in vacuo. The obtained crude material was basified using saturated NaHCO₃ (till pH˜7) and washed by 15% methanol in dichloromethane (1000 mL×2). The combined organic layer was dried over sodium sulphate and concentrated in vacuo. The obtained gum was triturated with diethyl ether and dried to afford 4-(4-methoxy-1-piperidyl)-4-methyl-cyclohexanone as a light-yellow oil (1.67 g, 39% yield) LCMS; 226.19 [M+1].

### Step-3: Synthesis of methyl-5-(4-methoxy-1-piperidyl)-5-methyl-2-oxidanylidene-cyclohexanecarboxylate (6)

To a stirred solution of 4-(4-methoxy-1-piperidyl)-4-methyl-cyclohexanone (1.0 g, 4.43 mmol) in THF (25 mL) was added sodium hydride (in oil dispersion) 60% dispersion in mineral oil (510 mg, 22.18 mmol) at 0° C. under nitrogen atmosphere. To the mixture, dimethyl carbonate (20 ml) was added, and the resulting mixture was allowed to stir for 6 h at 80° C. After completion, the reaction mixture was quenched with ice-cooled water and washed with 10% Methanol-DCM. The combined organic layer was dried over Na₂SO₄ and concentrated in vacuo. The obtained crude material was purified by column chromatography using 0-10% methanol-DCM as eluent to afford methyl-5-(4-methoxy-1-piperidyl)-5-methyl-2-oxidanylidene-cyclohexanecarboxylate as yellow gum (0.51 g, 40% yield). LCMS: 284.2 [M+1].

### Step-4: Synthesis of 6-(4-methoxy-1-piperidyl)-2,6-di(methyl)-3,5,7,8-tetrahydroquinazolin-4-one (8)

To a stirred solution of methyl 5-(4-methoxy-1-piperidyl)-5-methyl-2-oxidanylidene-cyclohexanecarboxylate (0.5 g, 1.76 mmol) in methanol (5 mL) Sodium methoxide solution (476.69 mg, 8.82 mmol) was added followed by the addition of acetamidine hydrochloride (166.82 mg, 1.76 mmol). and the resulting mixture was allowed to stir for 6 h at 80° C. After completion, the reaction mixture was concentrated to remove the volatiles completely. The pH of the reaction mixture was adjusted to neutral with saturated citric acid and then washed with 10% methanol in dichloromethane (3×20 mL). The organic layer was dried over Na₂SO₄ and concentrated in vacuo to afford 6-(4-methoxy-1-piperidyl)-2,6-di(methyl)-3,5,7,8-tetrahydroquinazolin-4-one (0.2 g, 39% yield) and obtained solid was used directly to next step. LCMS: 292.26 [M+1].

### Step 5: Synthesis of methyl 7-[5-chloranyl-2-[2-[6-(4-methoxy-1-piperidyl)-2,6-di(methyl)-4-oxidanylidene-7,8-dihydro-5H-quinazolin-3-yl]ethoxy]phenyl]-5-methyl-thieno[3,2-b]pyridine-3-carboxylate (10)

To a stirred solution of 6-(4-methoxy-1-piperidyl)-2,6-di(methyl)-3,5,7,8-tetrahydroquinazolin-4-one (0.5 g, 1.72 mmol) and methyl 7-[2-(2-bromanylethoxy)-5-chloranyl-phenyl]-5-methyl-thieno[3,2-b]pyridine-3-carboxylate (605.02 mg, 1.37 mmol) in N,N′-dimethyl formamide (5 mL) was added potassium carbonate-powder (711.45 mg, 5.15 mmol) and stirred the reaction mixture at 80° C. for 16 h. After completion, the reaction mixture was poured into ice-cold water and the obtained solid was filtered. The obtained crude material was purified by reverse phase column chromatography using ACN-water as eluent to afford methyl 7-[5-chloranyl-2-[2-[6-(4-methoxy-1-piperidyl)-2,6-di(methyl)-4-oxidanylidene-7,8-dihydro-5H-quinazolin-3-yl]ethoxy]phenyl]-5-methyl-thieno[3,2-b]pyridine-3-carboxylate as a white solid (0.125 g, 11.2% yield). LCMS: 651.31 [M+1].

### Step-6: Synthesis of 7-(5-chloro-2-(2-(6-(4-methoxypiperidin-1-yl)-2,6-dimethyl-4-oxo-5,6,7,8-tetrahydroquinazolin-3(4H)-yl)ethoxy)phenyl)-5-methylthieno[3,2-b]pyridine-3-carboxylic acid (11)

To a stirred solution of methyl 7-[5-chloranyl-2-[2-[6-(4-methoxy-1-piperidyl)-2,6-di(methyl)-4-oxidanylidene-7,8-dihydro-5H-quinazolin-3-yl]ethoxy]phenyl]-5-methyl-thieno[3,2-b]pyridine-3-carboxylate (100.00 mg, 153.56 μmol) in tetrahydrofuran (3 mL) and water (1 mL) was added lithium hydroxide (19.33 mg, 460.68 μmol) at 0° C. and resulting reaction was stirred at 25° C. for 2 h. Reaction was monitored by TLC and LCMS. After completion, the reaction mixture was concentrated to remove the volatiles completely. The pH of the reaction mixture was adjusted to neutral with 1N HCl and then washed with 10% methanol in dichloromethane (3×20 mL). The combined organics was washed with water and brine, dried over sodium sulphate. Dried organics was filtrated and concentrated under reduced pressure to get racemic 7-(5-chloro-2-(2-(6-(4-methoxypiperidin-1-yl)-2,6-dimethyl-4-oxo-5,6,7,8-tetrahydroquinazolin-3(4H)-yl)ethoxy)phenyl)-5-methylthieno[3,2-b]pyridine-3-carboxylic acid as an off-white solid. The chiral isomers were separated by SFC to afford 7-[5-chloranyl-2-[2-[(6S)-6-(4-methoxy-1-piperidyl)-2,6-di(methyl)-4-oxidanylidene-7,8-dihydro-5H-quinazolin-3-yl]ethoxy]phenyl]-5-methyl-thieno[3,2-b]pyridine-3-carboxylic acid as off-white solid (21 mg, 32.23 μmol, 21% yield). 7-[5-chloranyl-2-[2-[(6R)-6-(4-methoxy-1-piperidyl)-2,6-di(methyl)-4-oxidanylidene-7,8-dihydro-5H-quinazolin-3-yl]ethoxy]phenyl]-5-methyl-thieno[3,2-b]pyridine-3-carboxylic acid as an off-white solid (15 mg, 22.72 μmol, 15% yield). LCMS: 637.34 [M+1].

Below ester analogues are prepared using above protocol. Respective esters are hydrolysed to final compounds.

| | | | Ester 10 LCMS |
|---|---|---|---|
| Ex | Compound | Amine | [M + H]⁺ |
| 1 | Compound 567 Compound 566 | 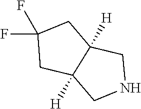 | 652.4 |
| 2 | Compound 600 Compound 599 | 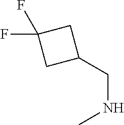 | 671.2 |

### 5. Experimental Procedure for Compound 565, Compound 559, Compound 558 Synthetic Scheme

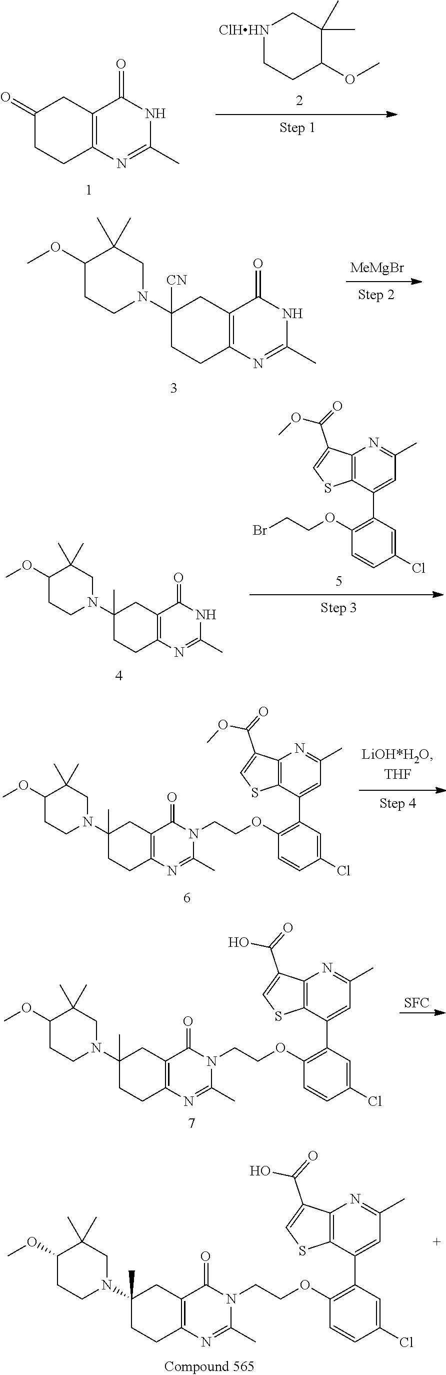

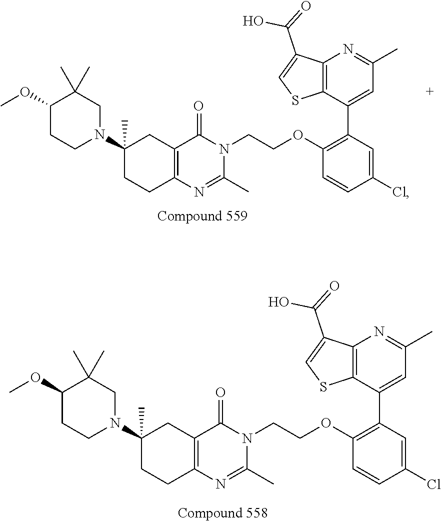 [SMILES: CO[C@H]1CCN([C@]2(C)CCc3nc(C)n(CCOc4ccc(Cl)cc4-c4cc(C)nc5c(C(=O)O)csc45)c(=O)c3C2)CC1(C)C ; CO[C@@H]1CCN([C@]2(C)CCc3nc(C)n(CCOc4ccc(Cl)cc4-c4cc(C)nc5c(C(=O)O)csc45)c(=O)c3C2)CC1(C)C]

### Step-1: Synthesis of 6-[4-methoxy-3,3-di(methyl)-1-piperidyl]-2-methyl-4-oxidanylidene-3,5,7,8-tetrahydroquinazoline-6-carbonitrile (3)

To a stirred solution of 4-methoxy-3,3-di(methyl)piperidine (1.21 g, 6.73 mmol) in DCE (8.93 mL) and add Triethylamine (851.83 mg, 8.42 mmol, 1.17 mL) at room temperature. Stirred for 15 mins. Then added 2-methyl-3,5,7,8-tetrahydroquinazoline-4,6-dione (1 g, 5.61 mmol) at room temperature. The resulting reaction mixture was stirred at ROOM TEMPERATURE for 12 h. Then slowly added potassium cyanide (548.15 mg, 8.42 mmol) and methanol (893.33 μL) at room temperature. Then the reaction mixture was stirred for 4 h and monitored by LCMS. After completion of the reaction, it was quenched by water (50 mL) and washed with DCM. Collected organics, dried over sodium sulphate and concentrated under reduced pressure to get 6-[4-methoxy-3,3-di(methyl)-1-piperidyl]-2-methyl-4-oxidanylidene-3,5,7,8-tetrahydroquinazoline-6-carbonitrile (1.2 g, 907.93 μmol, 16.18% yield, 25% purity). LCMS: 331.35 [M+1].

### Step-2: Synthesis of 6-[4-methoxy-3,3-di(methyl)-1-piperidyl]-2,6-di(methyl)-3,5,7,8-tetrahydroquinazolin-4-one (4)

To a stirred solution of 6-[4-methoxy-3,3-di(methyl)-1-piperidyl]-2-methyl-4-oxidanylidene-3,5,7,8-tetrahydroquinazoline-6-carbonitrile (1.2 g, 3.63 mmol) in THF (12 mL) was added Methyl magnesium bromide (3M in Et20, 1.69 g, 14.53 mmol, 1.63 mL) at −75° C. under N₂ atmosphere. Resulting reaction mixture was stirred at room temperature for 16 h. Reaction was monitored by LCMS. After completion of the reaction, slowly added cold water (20 mL) and washed with ethyl acetate (20 mL×3). Collected organics, dried over sodium sulphate and concentrated under reduced pressure to get crude material. Crude material was purified by reverse phase column with 50% ACN and 1% ammonium bicarbonate solution to afford 6-[4-methoxy-3,3-di(methyl)-1-piperidyl]-2,6-di(methyl)-3,5,7,8-tetrahydroquinazolin-4-one (400 mg, 713.75 μmol, 19.65% yield, 57% purity). LCMS: 320.22 [M+1].

### Step-3: Synthesis of methyl 7-[5-chloranyl-2-[2-[6-[4-methoxy-3,3-di(methyl)-1-piperidyl]-2,6-di(methyl)-4-oxidanylidene-7,8-dihydro-5H-quinazolin-3-yl]ethoxy]phenyl]-5-methyl-thieno[3,2-b]pyridine-3-carboxylate (6)

To a stirred solution of 6-[4-methoxy-3,3-di(methyl)-1-piperidyl]-2,6-di(methyl)-3,5,7,8-tetrahydroquinazolin-4-one (800 mg, 2.50 mmol) in DMF (546.56 μL) were added potassium carbonate (1.04 g, 7.51 mmol, 453.43 μL) and methyl 7-[2-(2-bromanylethoxy)-5-chloranyl-phenyl]-5-methyl-thieno[3,2-b]pyridine-3-carboxylate (1.10 g, 2.50 mmol). Resulting mixture was heated at 80° C. for 4 h. Reaction was monitored by LCMS. After completion of the reaction, reaction was quenched by cold water (20 mL) and then washed with ethyl acetate (20 mL×3). Collected organics, dried over sodium sulphate and concentrated under reduced pressure to obtain crude material. Crude material was purified by reverse phase column with 50% ACN and 1% ammonium bicarbonate solution afforded methyl 7-[5-chloranyl-2-[2-[6-[4-methoxy-3,3-di(methyl)-1-piperidyl]-2,6-di(methyl)-4-oxidanylidene-7,8-dihydro-5H-quinazolin-3-yl]ethoxy]phenyl]-5-methyl-thieno[3,2-b]pyridine-3-carboxylate (120 mg, 713.75 μmol, 19.65% yield, 52% purity). LCMS: 679.38 [M+1].

### Step-4: Synthesis of 7-[5-chloranyl-2-[2-[(6R)-6-[(4S)-4-methoxy-3,3-dimethyl-1-piperidyl]-2,6-di(methyl)-4-oxidanylidene-7,8-dihydro-5H-quinazolin-3-yl]ethoxy]phenyl]-5-methyl-thieno[3,2-b]pyridine-3-carboxylic acid (7)

To stirred solution of methyl 7-[5-chloranyl-2-[2-[6-[4-methoxy-3,3-di(methyl)-1-piperidyl]-2,6-di(methyl)-4-oxidanylidene-7,8-dihydro-5H-quinazolin-3-yl]ethoxy]phenyl]thieno[3,2-b]pyridine-3-carboxylate (120 mg, 180.39 μmol) in THF (5 mL) and water (3 mL) was add lithium hydroxide (12.96 mg, 541.16 μmol). The reaction mixture was stirred at 25° C. for 2 h. The progress of the reaction was monitored by LCMS. After the completion, the volatiles were evaporated under reduced pressure and the obtained crude material was acidified by 1M citric acid and washed with 10% methanol-DCM. The organic layer was dried over Na₂SO₄ and concentrated in vacuo. The obtained crude material compound was purified by prep-HPLC and room temperature isomer separation were carried by SFC-purification afforded each isomers. LCMS: 665.33 [M+1].

### 6. Experimental Procedure for Compound 516, Compound 515

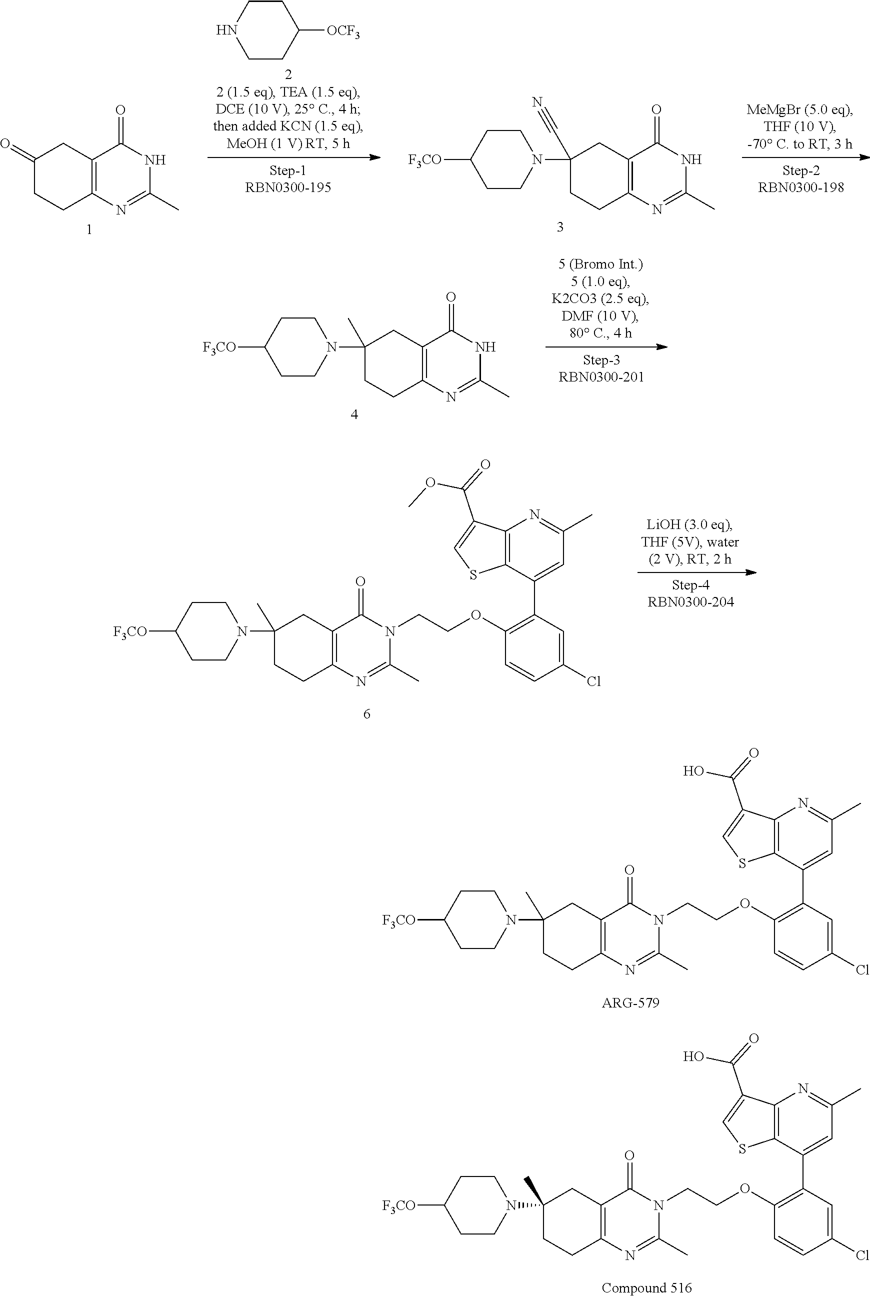

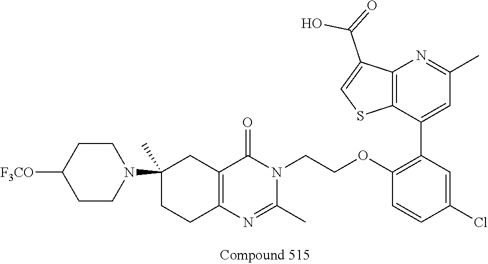 [SMILES: Cc1cc(-c2cc(Cl)ccc2OCCn2c(C)nc3c(c2=O)C[C@](C)(N2CCC(OC(F)(F)F)CC2)CC3)c2scc(C(=O)O)c2n1]

### Step-1: Synthesis of 2-methyl-4-oxo-6-(4-(trifluoromethoxy)piperidin-1-yl)-3,4,5,6,7,8-hexa hydroquinazoline-6-carbonitrile (3)

Triethyl Amine (2.92 g, 28.81 mmol, 4.02 mL) was added to the solution of 2-methyl-3,5,7,8-tetra hydroquinazoline-4,6-dione (3.5 g, 19.21 mmol) and 4-[tris(fluorenyl)methoxy]piperidine (4.87 g, 28.81 mmol) in DCE (100 mL) at room temperature and stirred for 3 h. Reaction mixture was cooled to 0° C. and KCN (1.99 g, 28.81 mmol) was added portion wise and allowed to warm at room temperature for 5 h. The reaction mixture was cooled to 0° C. and quenched with water and extract with DCM (100 mL×3). The combined organic layer was washed with cold water (100 mL) and brine solution (100 mL), the organic layer was dried over Na₂SO₄ and filtered. The filtrate was concentrated under reduced pressure to afford 2-methyl-4-oxo-6-(4-(trifluoromethoxy)piperidin-1-yl)-3,4,5,6,7,8-hexahydroquinazoline-6-carbonitrile (3.5 g, 50%) as brown gummy solid. LCMS; 357.27 [M+1].

### Step-2: Synthesis of 2,6-dimethyl-6-(4-(trifluoromethoxy)piperidin-1-yl)-5,6,7,8-tetrahydro quinazolin-4(3H)-one (4)

Methyl Magnesium Bromide (1.30 g, 11.23 mmol, 1.26 mL) was added slowly dropwise to the solution of 2-methyl-4-oxidanylidene-6-[4-[tris(fluorenyl)methoxy]-1-piperidyl]-3,5,7,8-tetrahydro quinazoline-6-carbonitrile (800 mg, 2.25 mmol) in THF (10 mL) at −78° C. and the resulting reaction mixture allowed to room temperature and stirred for 2 hr. The reaction mixture was cooled to 0° C. and quenched with sat. aq. ammonium chloride solution. Then extract with ethyl acetate (100 mL×3). The combined organic layer was washed with cold water (100 mL) and brine solution (100 mL), the organic layer was dried over Na₂SO₄ and filtered. The filtrate was concentrated under reduced pressure to afford crude. The crude was purified by combi-flash column chromatography using 230-400 mesh silica gel and the desired product was eluted at 0-10% of MeOH in DCM as the eluent. Pure fractions (determined by TLC) were combined and evaporated under reduced pressure to afford 2,6-dimethyl-6-(4-(trifluoromethoxy)piperidin-1-yl)-5,6,7,8-tetrahydro quinazolin-4(3H)-one (180 mg, 46%) as a brown solid. LCMS; 346.18 [M+1].

### Step 3: Synthesis of methyl 7-(5-chloro-2-(2-(2,6-dimethyl-4-oxo-6-(4-(trifluoro methoxy) piperidin-1-yl)-5,6,7,8-tetrahydroquinazolin-3(4H)-yl)ethoxy)phenyl)-5-methylthieno[3,2-b]pyridine-3-carboxylate (6)

Potassium carbonate (180 mg, 1.30 mmol, 78.64 μL) was added to the solution of 2,6-di(methyl)-6-[4-[tris(fluorenyl)methoxy]-1-piperidyl]-3,5,7,8-tetrahydroquinazolin-4-one (180 mg, 521.20 μmol)

in DMF (5 mL) at room temperature and slowly heated to 75° C. and stirred for 15 min. Then 1,1-di(methyl)ethyl 7-[2-(2-bromanylethoxy)-5-chloranyl-phenyl]-5-methyl-thieno[3,2-b]pyridine-3-carboxylate (252 mg, 521.20 μmol) was added slowly portion wise to the reaction mixture at 75° C. and the resulting reaction mixture was stirred for another 4 hr at same temperature. The reaction mixture was cooled to room temperature and quenched the water and extract with ethyl acetate (100 mL×3). The combined organic layer was washed with cold water (100 mL) and brine solution (100 mL), the organic layer was dried over Na₂SO₄ and filtered. The filtrate was concentrated under reduced pressure to afford crude as brown liquid. The crude was purified by combi-flash column chromatography using 230-400 mesh silica gel and the desired product was eluted at 0-10% of MeOH in DCM as the eluent. Pure fractions (determined by TLC) were combined and evaporated under reduced pressure to afford methyl 7-(5-chloro-2-(2-(2,6-dimethyl-4-oxo-6-(4-(trifluoro methoxy) piperidin-1-yl)-5,6,7,8-tetrahydroquinazolin-3(4H)-yl)ethoxy)phenyl)-5-methylthieno[3,2-b]pyridine-3-carboxylate (120 mg, 35%) as brown solid. LCMS; 705.23 [M+1].

### Step 4: Synthesis of (S)-7-(5-chloro-2-(2-(2,6-dimethyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylic acid and (R)-7-(5-chloro-2-(2-(2,6-dimethyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylic acid

Lithium hydroxide monohydrate (36 mg, 850.84 μmol, 23.65 μL) was added to the solution of methyl 7-[5-chloranyl-2-[2-[2,6-di(methyl)-4-oxidanylidene-6-[4-[tris(fluorenyl)methoxy]-1-piperidyl]-7,8-dihydro-5H-quinazolin-3-yl]ethoxy]phenyl]-5-methyl-thieno[3,2-b]pyridine-3-carboxylate (120 mg, 170.17 μmol) in a mixture of solvents THE and H₂O (2 mL and 0.5 mL) at room temperature. The resulting mixture was stirred at room temperature for 2 h. The resulting reaction mixture was concentrated at 45° C. under reduced pressure. This reaction mass was diluted in water and acidified with sat. aq. citric acid solution to pH 4-6. Then washed 10% MeOH in dichloromethane (30 mL×2). The combined organic layer was concentrated under reduced pressure to obtain the crude material. Crude was purified by Prep-HPLC (Column Name X-BRIDGE-C18 (150*19 mm), 5u Column No #MCL-PREP-COL-2022-068 Mobile Phase-A 10 mM Ammonium Bicarbonate in water Mobile Phase-B Acetonitrile Gradient program (T/% B) 0/15, 12/50, 12.1/98, 14/98, 14.1/15, 16/15 Flow Rate (mL/minute) 16 Sample Loading(mg/Injection) to afford desired product as an off white solid. Isomer was separated by SFC to afford two desired isomers as an off white solid. LCMS; 691.25 [M+1].

Below ester analogues are prepared using above protocol and hydrolysed to final compounds

| | | | Ester 5 |
|---|---|---|---|
| Ex | Compound | Amine | LCMS [M + H]⁺ |
| 1 | Compound 531 Compound 530 | 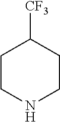 | 689.7 |
| 2 | Compound 555 Compound 539 Compound 554 Compound 553 | 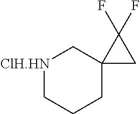 | 683.2 |
| 3 | Compound 557 Compound 556 | 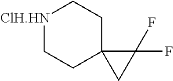 | 683.2 |

### 7. Experimental Procedure for Compound 239 and Compound 240

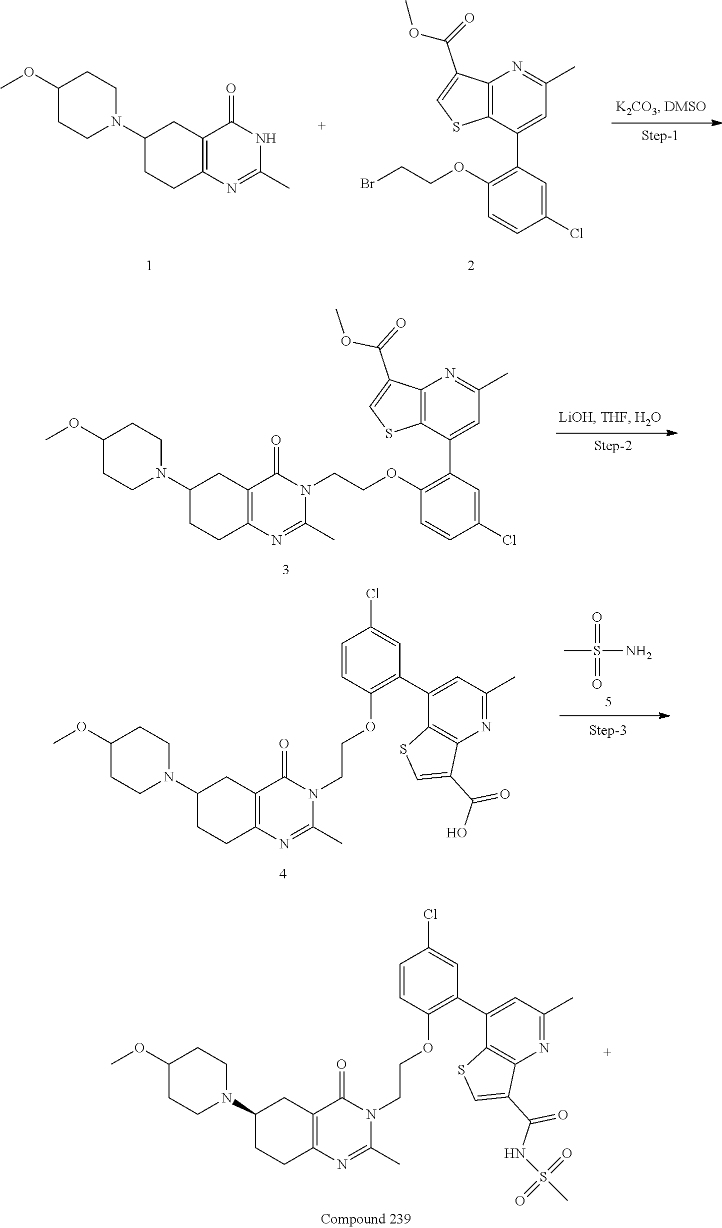

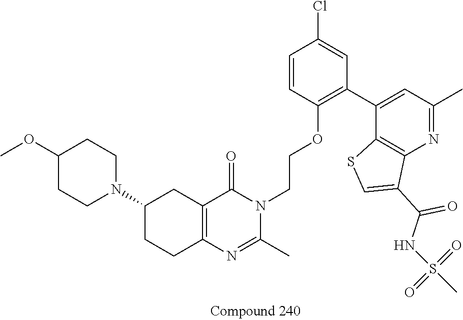

### Step-1: Synthesis of methyl 7-(5-chloro-2-(2-(6-(4-methoxypiperidin-1-yl)-2-methyl-4-oxo-5,6,7,8-tetrahydroquinazolin-3(4H)-yl)ethoxy)phenyl)-5-methylthieno[3,2-b]pyridine-3-carboxylate (3)

This compound was synthesised by common procedure E from 6-(4-methoxy-1-piperidyl)-2-methyl-5,6,7,8-tetrahydro-3H-quinazolin-4-one (1.4 g, 5.05 mmol) to afford methyl 7-(5-chloro-2-(2-(6-(4-methoxypiperidin-1-yl)-2-methyl-4-oxo-5,6,7,8-tetrahydroquinazolin-3(4H)-yl)ethoxy)phenyl)-5-methylthieno[3,2-b]pyridine-3-carboxylate. LCMS: 637.25.

### Step-2: Synthesis of 7-(5-chloro-2-(2-(6-(4-methoxypiperidin-1-yl)-2-methyl-4-oxo-5,6,7,8-tetrahydroquinazolin-3(4H)-yl)ethoxy)phenyl)-5-methylthieno[3,2-b]pyridine-3-carboxylic acid (4)

This compound was synthesised by common procedure B from methyl 7-[5-chloranyl-2-[2-[6-(4-methoxy-1-piperidyl)-2-methyl-4-oxidanylidene-5,6,7,8-tetrahydroquinazolin-3-yl]ethoxy]phenyl]-5-methyl-thieno[3,2-b]pyridine-3-carboxylate (650 mg, 1.02 mmol) afforded 7-(5-chloro-2-(2-(6-(4-methoxypiperidin-1-yl)-2-methyl-4-oxo-5,6,7,8-tetrahydroquinazolin-3(4H)-yl)ethoxy)phenyl)-5-methylthieno[3,2-b]pyridine-3-carboxylic acid. LCMS: 623.16.

### Step 3: Synthesis of (R)-7-(5-chloro-2-(2-(6-(4-methoxypiperidin-1-yl)-2-methyl-4-oxo-5,6,7,8-tetrahydroquinazolin-3(4H)-yl)ethoxy)phenyl)-5-methyl-N-(methylsulfonyl)thieno[3,2-b]pyridine-3-carboxamide and (S)-7-(5-chloro-2-(2-(6-(4-methoxypiperidin-1-yl)-2-methyl-4-oxo-5,6,7,8-tetrahydroquinazolin-3(4H)-yl)ethoxy)phenyl)-5-methyl-N-(methylsulfonyl)thieno[3,2-b]pyridine-3-carboxamide

These intermediated are synthesised using procedure F1 from of 7-[5-chloranyl-2-[2-[6-(4-methoxy-1l-piperidyl)-2-methyl-4-oxidanylidene-5,6,7,8-tetrahydroquinazolin-3-yl]ethoxy]phenyl]-5-methyl-thieno[3,2-b]pyridine-3-carboxylic acid to afford 7-[5-chloranyl-2-[2-[(6R)-6-(4-methoxy-1l-piperidyl)-2-methyl-4-oxidanylidene-5,6,7,8-tetrahydroquinazolin-3-yl]ethoxy]phenyl]-5-methyl-N-methylsulfonyl-thieno[3,2-b]pyridine-3-carboxamide (42.46 mg, 60.33 μmol, 6.27% yield, 99.5000 purity) 7-[5-chloranyl-2-[2-[(6S)-6-(4-methoxy-1-piperidyl)-2-methyl-4-oxidanylidene-5,6,7,8-tetrahydroquinazolin-3-yl]ethoxy]phenyl]-5-methyl-N-methylsulfonyl-thieno[3,2-b]pyridine-3-carboxamide (38.23 mg, 53.69 μmol, 5.58% yield, 98.34% purity) as an off white solid. LCMS: 700.27 [M+1].

The following analogues are prepared using the above protocol.

| | | | Sulfonamide LCMS |
|---|---|---|---|
| Ex | Compound | Amine | [M + H]⁺ |
| 1 | Compound 214 Compound 215 | 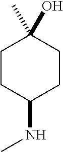 | 728.2 |
| 2 | Compound 104 Compound 105 | 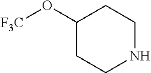 [SMILES: FC(F)(F)OC1CCNCC1] | 754.37 |
| 3 | Compound 433 Compound 432 | 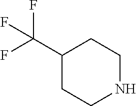 | 738.28 |
| 4 | Compound 133 | 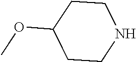 | 622.3 |
| 5 | Compound 364 Compound 363 | 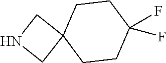 [SMILES: FC1(F)CCC2(CC1)CNC2] | 746.2 |
| 6 | Compound 417 | 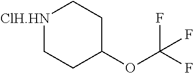 | 780.30 |
| 7 | Compound 459 | 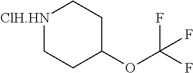 | 808.2 |
| 8 | Compound 416 | 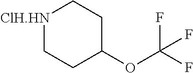 | 790.2 |

### 8. Experimental Procedure for Compound 592

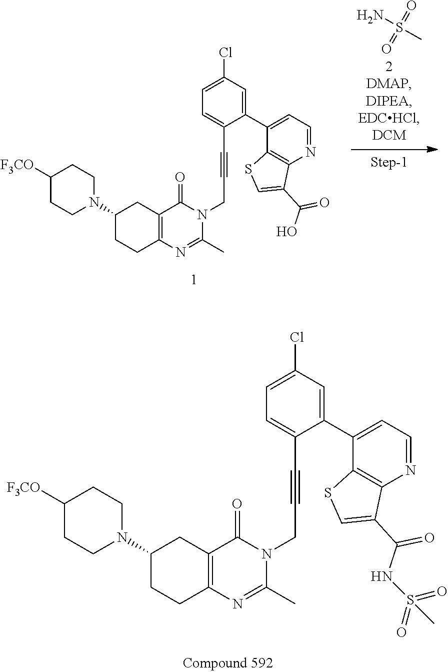

### Step 1: Synthesis of (S)-7-(5-chloro-2-(3-(2-methyl-4-oxo-6-(4-(trifluoromethoxy)piperidin-1-yl)-5,6,7,8-tetrahydroquinazolin-3(4H)-yl)prop-1-yn-1-yl)phenyl)-N-(methylsulfonyl)thieno[3,2-b]pyridine-3-carboxamide)

To the solution of 7-[5-chloranyl-2-[3-[(6S)-2-methyl-4-oxidanylidene-6-[4-(trifluoromethoxy)-1-piperidyl]-5,6,7,8-tetrahydroquinazolin-3-yl]prop-1-ynyl]phenyl]thieno[3,2-b]pyridine-3-carboxylic acid (50 mg, 76.09 μmol) in DCM (1 mL) was added DMAP (18.59 mg, 152.18 μmol), DIPEA (49.17 mg, 380.45 μmol, 66.27 μL), EDC·HCl (72.93 mg, 380.46 μmol) at room temperature. The resulting reaction mixture was stirred at room temperature for 20 min. Then was added methane sulfonamide (36.19 mg, 380.46 mol) at same temperature. The resulting reaction mixture was stirred at 25° C. for 12 hr. The progress of reaction was monitored by LCMS. LCMS showed mass of desired product. After completion of starting material, reaction was quenched with water (2 mL) and washed with DCM (3×5 mL). The combined organic layer was dried over Na₂SO₄, filtered and concentrated to get the crude compound. Purification was done by Prep-HPLC (Column: X-BRIDGE-C18 (150*30), 5u Mobile phase: 10 mM Ammonium Bi Carbonate in H₂O:ACETONITRILE GRADIENT: (T % B): 0/10, 10/50, 13.7/50, 13.8/98, 16/98, 16.1/10, 18/10 Flow Rate: 16 ml/min Diluent: ACN+H2O) to afford desired product as a white solid. LCMS; 734.27 [M+1].

The following analogues are synthesized using above protocol.

| | | | | LCMS |
|---|---|---|---|---|
| Ex | Compound | Amine | Method | [M + H]⁺ |
| 1 | Compound 414 | 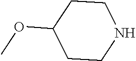 | F1 | 680.3 |

### 9. Experimental Procedure for Compound 158

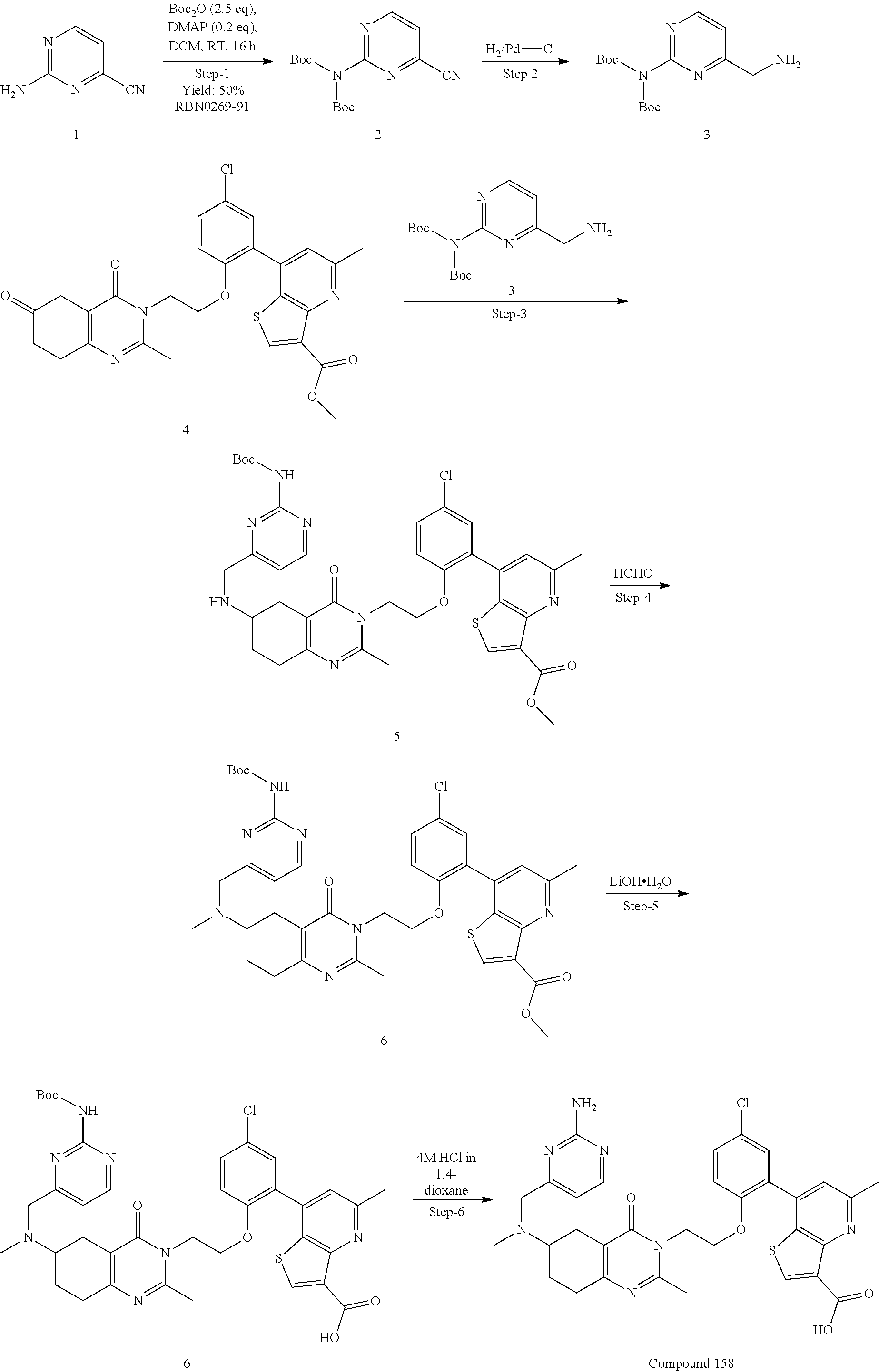

### Step-1: Synthesis of tert-butyl (tert-butoxycarbonyl)(4-cyanopyrimidin-2-yl)carbamate (2)

To a stirred solution of 2-azanylpyrimidine-4-carbonitrile (0.750 g, 6.24 mmol) in DCM (10 mL) at 0° C. were added DMAP (762.84 mg, 6.24 mmol) and boc-anhydride (3.41 g, 15.61 mmol, 3.58 mL) and resulting reaction mixture was stirred at 25° C. for 18 h. Progressing of the reaction was monitored by TLC. The reaction mixture was washed with DCM the layer was washed with water and the organic layer was dried using anhydrous sodium sulphate the organic layer was concentrated to get a crude product purified by flash chromatography by using ethyl acetate and hexane pure compound was fraction collected concentrated 1,1-di(methyl)ethyl N-[4-(azanylidynemethyl)pyrimidin-2-yl]-N-[1,1-di(methyl)ethoxycarbonyl]carbamate (1 g, 3.12 mmol, 49.99% yield). LCMS: 321 [M+1].

### Step-2: Synthesis of tert-butyl (4-(aminomethyl)pyrimidin-2-yl)(tert-butoxycarbonyl)carbamate (3)

To a stirred solution of 1,1-di(methyl)ethyl N-[4-(azanylidynemethyl)pyrimidin-2-yl]-N-[1,1-di(methyl)ethoxycarbonyl]carbamate (1 g, 3.12 mmol) in methanol (10 mL) added palladium (10% on carbon, Type 487, dry, 332.21 mg, 3.12 mmol) under hydrogen (50-60 Psi) atmosphere. The reaction was heated to 75° C. for 4 h. Progressing of the reaction was monitored by TLC and LCMS. The reaction mixture was cooled to 25° C. and mixture was filtered through cellite bed and filtrate was concentrated under reduced pressure to obtain 1,1-di(methyl)ethyl N-[4-(azanylmethyl)pyrimidin-2-yl]-N-[1,1-di(methyl)ethoxycarbonyl]carbamate (750 mg, 2.31 mmol, 74.07% yield) as a brown semi-solid. LCMS: 325.26.

### Step-3: Synthesis of methyl 7-(2-(2-(6-(((2-((tert-butoxycarbonyl)amino)pyrimidin-4-yl)methyl)amino)-2-methyl-4-oxo-5,6,7,8-tetrahydroquinazolin-3(4H)-yl)ethoxy)-5-chlorophenyl)-5-methylthieno[3,2-b]pyridine-3-carboxylate (5)

To a stirred solution of methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (400 mg, 743.48 μmol) in methanol (0.5 mL) was added in 1,1-di(methyl)ethyl N-[4-(azanylmethyl)pyrimidin-2-yl]-N-[1,1-di(methyl)ethoxycarbonyl]carbamate (442.13 mg, 817.82 mol). The resulting reaction mixture was stirred for 30 minutes at 25° C. Afterwards Sodium cyanoborohydride (116.80 mg, 1.86 mmol) was added to this and the resulting reaction mixture was stirred at the same temperature for 18 h. Reaction was monitored by TLC. The reaction mixture was poured into ice-cold water (15 mL) and filtered through a celite bed followed by washing with 10% methanol in dichloromethane (15 mL). The organic layer was separated, and the aqueous layer was washed again with 10% methanol in dichloromethane (15 mL). The combined organic layer was concentrated under reduced pressure to obtain crude material. The crude was used for the next step without further purification. LCMS: 746.30 [M+1].

### Step 4: methyl 7-(2-(2-(6-(((2-((tert-butoxycarbonyl)amino)pyrimidin-4-yl)methyl)(methyl)amino)-2-methyl-4-oxo-5,6,7,8-tetrahydroquinazolin-3(4H)-yl)ethoxy)-5-chlorophenyl)-5-methylthieno[3,2-b]pyridine-3-carboxylate

To a stirred solution of methyl 7-[5-chloranyl-2-[2-[6-[[2-[1,1-di(methyl)ethoxycarbonylamino]pyrimidin-4-yl]methylamino]-2-methyl-4-oxidanylidene-5,6,7,8-tetrahydroquinazolin-3-yl]ethoxy]phenyl]-5-methyl-thieno[3,2-b]pyridine-3-carboxylate (360 mg, 482.40 μmol) in methanol (5.01 mL) was added formaldehyde (37% w/w aq. soln., stab. with 7-8% methanol, 72.42 mg, 2.41 mmol, 66.87 μL). The resulting reaction mixture was stirred for 30 minutes at 25° C. To this, Sodium cyanoborohydride (75.79 mg, 1.21 mmol) was added and the resulting reaction mixture was stirred for 4 h at the same temperature. After complete conversion of starting to desired product, reaction mixture was concentrated to get crude compound. The crude compound was purified by Biotage 12 g C18 cartridge and the compound was eluted with a gradient of 0-100% 0.1 Ammonium acetate water in ACN. The pure fraction was collected and concentrated under reduced pressure to afford methyl 7-[5-chloranyl-2-[2-[6-[[2-[1,1-di(methyl)ethoxycarbonylamino]pyrimidin-4-yl]methyl-methyl-amino]-2-methyl-4-oxidanylidene-5,6,7,8-tetrahydroquinazolin-3-yl]ethoxy]phenyl]-5-methyl-thieno[3,2-b]pyridine-3-carboxylate (195 mg, 256.48 μmol, 53.17% yield). LCMS: 746.30 [M+1].

### Step-4: Synthesis of 7-(2-(2-(6-(((2-((tert-butoxycarbonyl)amino)pyrimidin-4-yl)methyl)(methyl)amino)-2-methyl-4-oxo-5,6,7,8-tetrahydroquinazolin-3(4H)-yl)ethoxy)-5-chlorophenyl)-5-methylthieno[3,2-b]pyridine-3-carboxylic acid (6)

To a stirred solution of methyl 7-[5-chloranyl-2-[2-[6-[[2-[1,1-di(methyl)ethoxycarbonylamino]pyrimidin-4-yl]methyl-methyl-amino]-2-methyl-4-oxidanylidene-5,6,7,8-tetrahydroquinazolin-3-yl]ethoxy]phenyl]-5-methyl-thieno[3,2-b]pyridine-3-carboxylate (195 mg, 256.48 μmol) in THF (5 mL) and water (2 mL) the reaction mixture was added in lithium hydroxide, monohydrate (53.81 mg, 1.28 mmol, 35.64 μL) at 0° C. Reaction stirred at room temperature for 2 h. After completion of the reaction, excess of solvent was evaporated, and crude material was washed with diethyl ether (2×20 mL). After that, reaction mass pH was adjusted to 6˜7 with 0.5 N HCl solution. Reaction mixture was washed by ethyl acetate, organics were dried over sodium sulphate and concentrated under reduced pressure to obtain 7-[5-chloranyl-2-[2-[6-[[2-[1,1-di(methyl)ethoxycarbonylamino]pyrimidin-4-yl]methyl-methyl-amino]-2-methyl-4-oxidanylidene-5,6,7,8-tetrahydroquinazolin-3-yl]ethoxy]phenyl]-5-methyl-thieno[3,2-b]pyridine-3-carboxylic acid (174 mg, 233.16 μmol, 90.91% yield). LCMS: 746.29.

### Step-5: Synthesis of 7-(2-(2-(6-(((2-aminopyrimidin-4-yl)methyl)(methyl)amino)-2-methyl-4-oxo-5,6,7,8-tetrahydroquinazolin-3(4H)-yl)ethoxy)-5-chlorophenyl)-5-methylthieno[3,2-b]pyridine-3-carboxylic acid (Compound 158)

To a stirred solution of 7-[5-chloranyl-2-[2-[6-[[2-[1,1-di(methyl)ethoxycarbonylamino]pyrimidin-4-yl]methyl-methyl-amino]-2-methyl-4-oxidanylidene-5,6,7,8-tetrahydroquinazolin-3-yl]ethoxy]phenyl]-5-methyl-thieno[3,2-b]pyridine-3-carboxylic acid (174 mg, 233.16 μmol) in 1,4-dioxane (2 mL) was added in 4.0 M hydrogen chloride in 1,4-dioxane (1 mL) at 0° C. After that, reaction mixture was stirred at 25° C. for 2 h. Progressing of the reaction was monitored by LCMS. The reaction mixture was concentrated under reduced pressure to get crude material. The crude material was purified by Prep-HPLC to afford 7-[2-[2-[6-[(2-azanylpyrimidin-4-yl)methyl-methyl-amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylic acid (40 mg, 60.02 μmol, 25.74% yield, 96.96% purity). LCMS: 646.24 [M+1].

The following analogues are prepared using above protocol.

| | | | LCMS |
|---|---|---|---|
| Ex | Compound | Amine | [M + H]⁺ |
| 1 | Compound 235 Compound 236 | 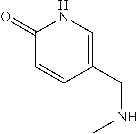 | 646.35 |
| 2 | Compound 255 | 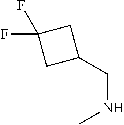 | 643.37 |
| 3 | Compound 183 Compound 184 | 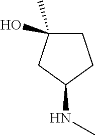 | 637.4 |
| 4 | Compound 200 | 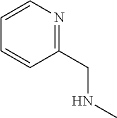 | 630.37 |
| 5 | Compound 202 | 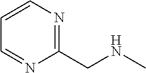 | 631.21 |
| 6 | Compound 190 | 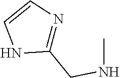 | 619.32 |
| 7 | Compound 191 | 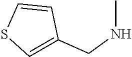 | 634.15 |
| 8 | Compound 205 | 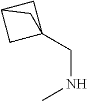 | 619.25 |
| 9 | Compound 193 | 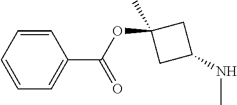 [SMILES: CN[C@H]1C[C@](C)(OC(=O)c2ccccc2)C1] | 741.2 |
| 10 | Compound 222 | 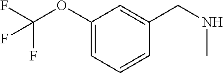 | 727.2 |
| 11 | Compound 229 Compound 228 | 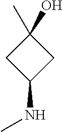 | 637.3 |
| 12 | Compound 493 | 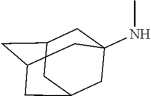 | 688.29 |

### 10. Experimental Procedure for Compound 125

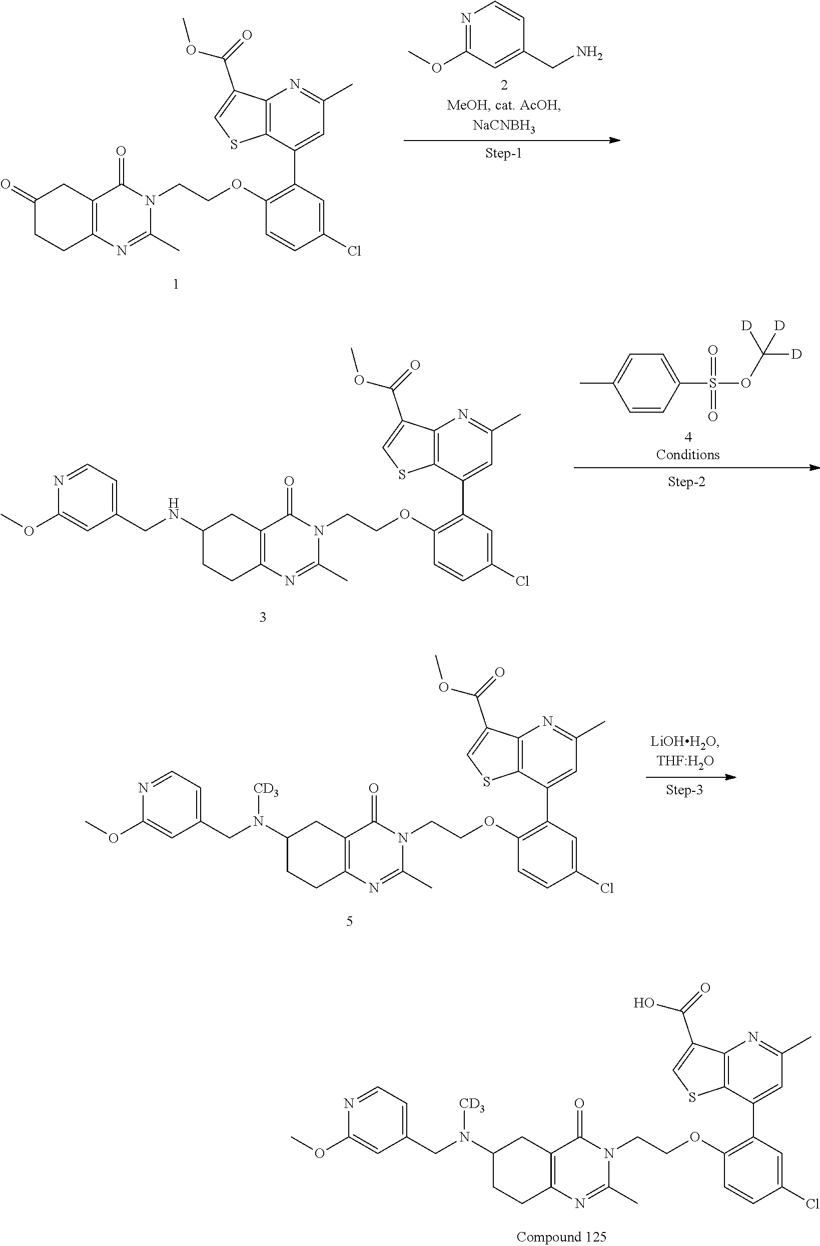

### Step-1: Synthesis of methyl 7-(5-chloro-2-(2-(6-(((2-methoxypyridin-4-yl)methyl)amino)-2-methyl-4-oxo-5,6,7,8-tetrahydroquinazolin-3(4H)-yl)ethoxy)phenyl)-5-methylthieno[3,2-b]pyridine-3-carboxylate (3)

This compound was prepared by common procedure A8 from of methyl 7-(5-chloro-2-(2-(2-methyl-4,6-dioxo-5,6,7,8-tetrahydroquinazolin-3(4H)-yl)ethoxy)phenyl)-5-methylthieno[3,2-b]pyridine-3-carboxylate (1 g, 1.86 mmol) and (2-methoxypyridin-4-yl)methanamine (256.81 mg, 1.86 mmol) afforded 7-(5-chloro-2-(2-(6-(((2-methoxypyridin-4-yl)methyl)amino)-2-methyl-4-oxo-5,6,7,8-tetrahydroquinazolin-3(4H)-yl)ethoxy)phenyl)-5-methylthieno[3,2-b]pyridine-3-carboxylate (350 mg, 440.03 μmol, 23.67% yield, 83% purity), LCMS: 660.19 [M+1].

### Step-2: Synthesis of methyl 7-(5-chloro-2-(2-(6-(((2-methoxypyridin-4-yl)methyl)(methyl-d3)amino)-2-methyl-4-oxo-5,6,7,8-tetrahydroquinazolin-3(4H)-yl)ethoxy)phenyl)-5-methylthieno[3,2-b]pyridine-3-carboxylate (5)

To a stirred solution of methyl 7-[5-chloranyl-2-[2-[6-[(2-methoxy-4-pyridyl)methylamino]-2-methyl-4-oxidanylidene-5,6,7,8-tetrahydroquinazolin-3-yl]ethoxy]phenyl]-5-methyl-thieno[3,2-b]pyridine-3-carboxylate (130 mg, 196.92 mol) and DMF (996.42 μL). Then slowly added potassium carbonate (8.30 mg, 36.03 μmol, 3.62 μL). This mixture was stirred for 15 minutes. After that, methyl-D3 toluene sulfonate (37.27 mg, 196.92 μmol) was added and resulting reaction mixture was stirred for 16 h at room temperature. The reaction was monitored by LCMS. After completion of the reaction, water was added to it and washed Ethyl acetate. Collected organics, dried over sodium sulphate and concentrated under reduced pressure to obtain methyl 7-[5-chloro-2-[2-[6-[(2-methoxy-4-pyridyl)methyl-(trideuteriomethyl)amino]-2-methyl-4-oxo-5,6,7,8-tetrahydroquinazolin-3-yl]ethoxy]phenyl]-5-methyl-thieno[3,2-b]pyridine-3-carboxylate (200 mg, Crude material). LCMS: 660.19

### Step-3: Synthesis of 7-(5-chloro-2-(2-(6-(((2-methoxypyridin-4-yl)methyl)(methyl-d3)amino)-2-methyl-4-oxo-5,6,7,8-tetrahydroquinazolin-3(4H)-yl)ethoxy)phenyl)-5-methylthieno[3,2-b]pyridine-3-carboxylic acid (Compound 125)

This product was prepared using procedure 2 using methyl 7-[5-chloro-2-[2-[6-[(2-methoxy-4-pyridyl)methyl-(trideuteriomethyl)amino]-2-methyl-4-oxo-5,6,7,8-tetrahydroquinazolin-3-yl]ethoxy]phenyl]-5-methyl-thieno[3,2-b]pyridine-3-carboxylate (200 mg, 295.32 μmol) afforded 7-[5-chloro-2-[2-[6-[(2-methoxy-4-pyridyl)methyl-(trideuteriomethyl)amino]-2-methyl-4-oxo-5,6,7,8-tetrahydroquinazolin-3-yl]ethoxy]phenyl]-5-methyl-thieno[3,2-b]pyridine-3-carboxylic acid (31.53 mg, 45.89 μmol, 15.54% yield, 96.53% purity). LCMS: 663.30 [M+1].

### 11. Experimental Procedure for Compound 112

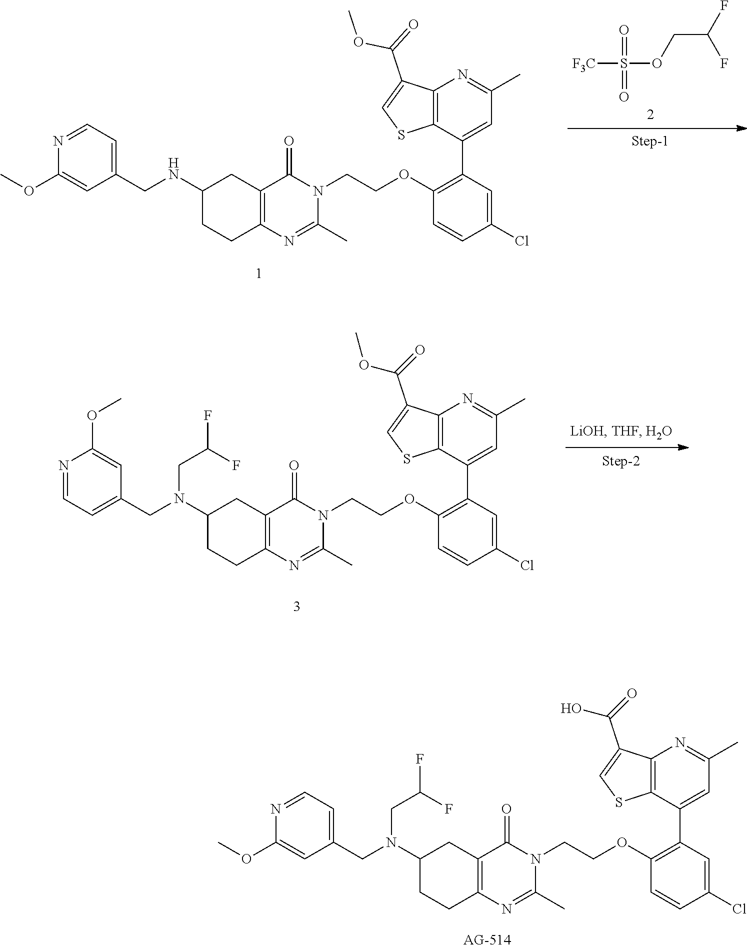 [SMILES: COC(=O)c1csc2c(-c3cc(Cl)ccc3OCCn3c(C)nc4c(c3=O)CC(NCc3ccnc(OC)c3)CC4)cc(C)nc12 ; O=S(=O)(OCC(F)F)C(F)(F)F ; COC(=O)c1csc2c(-c3cc(Cl)ccc3OCCn3c(C)nc4c(c3=O)CC(N(Cc3ccnc(OC)c3)CC(F)F)CC4)cc(C)nc12 ; COc1cc(CN(CC(F)F)C2CCc3nc(C)n(CCOc4ccc(Cl)cc4-c4cc(C)nc5c(C(=O)O)csc45)c(=O)c3C2)ccn1]

### Step-1: Synthesis of methyl 7-[2-[2-[6-[2,2-bis(fluorenyl)ethyl-[(2-methoxy-4-pyridyl)methyl]amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (3)

To a stirred solution of methyl 7-[5-chloranyl-2-[2-[6-[(2-methoxy-4-pyridyl)methylamino]-2-methyl-4-oxidanylidene-5,6,7,8-tetrahydroquinazolin-3-yl]ethoxy]phenyl]-5-methyl-thieno[3,2-b]pyridine-3-carboxylate (300 mg, 454.42 μmol) in DMF (894.48 μL) was added in potassium carbonate (314.02 mg, 2.27 mmol, 137.13 μL) and reaction mixture stirred at room temperature for 30 minutes. After that, 2,2-Difluoroethyl trifluoro methane sulfonate (972.97 mg, 4.54 mmol) was added and reaction mixture was stirred for another 16 h. The progress of reaction was monitored by LCMS. After completion of the reaction, mixture was quenched with cold water and washed with ethyl acetate. Collected organics, dried over sodium sulphate and concentrated under reduced pressure to obtain methyl 7-[2-[2-[6-[2,2-bis(fluorenyl)ethyl-[(2-methoxy-4-pyridyl)methyl]amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate. LCMS: 724 [M+1].

### Step-2: Synthesis of 7-[2-[2-[6-[2,2-bis(fluorenyl)ethyl-[(2-methoxy-4-pyridyl)methyl]amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylic acid (Compound 112)

This product was prepared by procedure B using methyl 7-[2-[2-[6-[2,2-bis(fluorenyl)ethyl-[(2-methoxy-4-pyridyl)methyl]amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylate (290 mg, 400.43 μmol) afforded 7-[2-[2-[6-[2,2-bis(fluorenyl)ethyl-[(2-methoxy-4-pyridyl)methyl]amino]-2-methyl-4-oxidanylidene-5,6,7,8-tetrahydroquinazolin-3-yl]ethoxy]-5-chloranyl-phenyl]-5-methyl-thieno[3,2-b]pyridine-3-carboxylic acid (7.54 mg, 10.56 μmol, 2.64% yield, 99.50% purity). LCMS: 683.32 [M+1].

### 12. Experimental Procedure for Compound 270

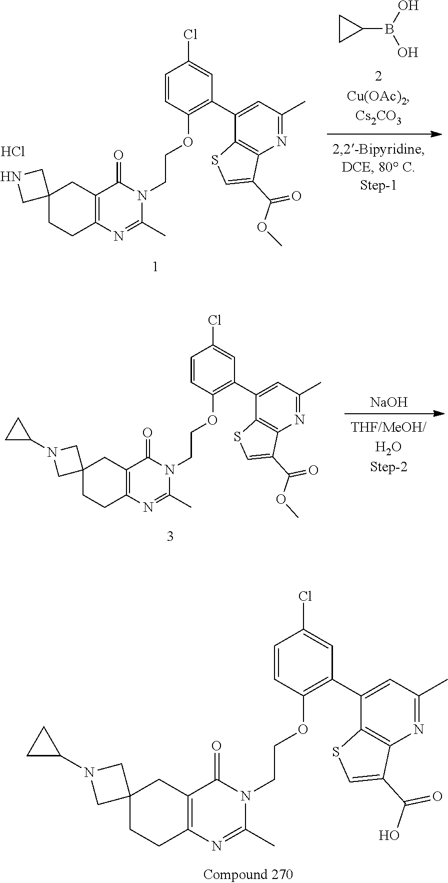 [SMILES: Cl ; COC(=O)c1csc2c(-c3cc(Cl)ccc3OCCn3c(C)nc4c(c3=O)CC3(CC4)CNC3)cc(C)nc12 ; OB(O)C1CC1 ; COC(=O)c1csc2c(-c3cc(Cl)ccc3OCCn3c(C)nc4c(c3=O)CC3(CC4)CN(C4CC4)C3)cc(C)nc12 ; "NaOH" ; Cc1cc(-c2cc(Cl)ccc2OCCn2c(C)nc3c(c2=O)CC2(CC3)CN(C3CC3)C2)c2scc(C(=O)O)c2n1]

### Step-1: Synthesis of methyl 7-(5-chloro-2-(2-(1-cyclopropyl-2′-methyl-4′-oxo-7′,8′-dihydro-4′H-spiro[azetidine-3,6′-quinazolin]-3′(5′H)-yl)ethoxy)phenyl)-5-methylthieno[3,2-b]pyridine-3-carboxylate (3)

To a stirred solution of Methyl 7-[5-chloranyl-2-[2-(2-methyl-4-oxidanylidene-spiro[7,8-dihydro-5H-quinazoline-6,3′-azetidine]-3-yl)ethoxy]phenyl]-5-methyl-thieno[3,2-b]pyridine-3-carboxylate (100 mg, 176.97 μmol), in DCE (3 mL) were added cyclo-propyl boronic acid (45.60 mg, 530.90 μmol), cesium carbonate (115.32 mg, 353.93 μmol), copper acetate (48.21 mg, 265.45 μmol), and 2,2 bipyridine (41.46 mg, 265.45 μmol). Resulting reaction mixture was stirred at 90° C. for 4 h. Progress of the reaction was monitored by LCMS. The reaction mixture was cooled to room temperature, poured into ice-cold water (10 mL) and stirred for 5 min at room temperature. The reaction mixture was then washed with 20 mL of ethyl acetate. The combined organic layer was concentrated under reduced pressure to obtain the crude material. The crude material was washed with n-hexanes (10 mL) and formed precipitate was filtered on Büchner funnel. The solid material was dried to afford methyl 7-[5-chloranyl-2-[2-(1′-cyclopropyl-2-methyl-4-oxidanylidene-spiro[7,8-dihydro-5H-quinazoline-6,3′-azetidine]-3-yl)ethoxy]phenyl]-5-methyl-thieno[3,2-b]pyridine-3-carboxylate as a white solid. LCMS; 605.26 [M+1].

### Step-2: Synthesis of 7-(5-chloro-2-(2-(1-cyclopropyl-2′-methyl-4′-oxo-7′,8′-dihydro-4′H-spiro[azetidine-3,6′-quinazolin]-3′(5′H)-yl)ethoxy)phenyl)-5-methylthieno[3,2-b]pyridine-3-carboxylic acid (Compound 270)

To a stirred solution of methyl 7-[5-chloranyl-2-[2-(1′-cyclopropyl-2-methyl-4-oxidanylidene-spiro[7,8-dihydro-5H-quinazoline-6,3′-azetidine]-3-yl)ethoxy]phenyl]-5-methyl-thieno[3,2-b]pyridine-3-carboxylate (120 mg, 198.30 μmol) in a mixture of THF/Methanol/H2O (0.5 mL/0.3 mL/0.3 mL) was added sodium hydroxide 50% (39.66 mg, 991.50 μmol, 18.62 μL) at room temperature. The resulting mixture stirred at 50° C. for 1 h. Progress of the reaction was monitored by TLC and LCMS. The reaction mass was acidified with 1 M HCl/sat. citric acid solution to pH 4-6 and washed by ethyl acetate. The volatiles were removed under reduced pressure to obtain the crude material. The crude material was purified by prep-HPLC to afford 7-[5-chloranyl-2-[2-(1′-cyclopropyl-2-methyl-4-oxidanylidene-spiro[7,8-dihydro-5H-quinazoline-6,3′-azetidine]-3-yl)ethoxy]phenyl]-5-methyl-thieno[3,2-b]pyridine-3-carboxylic acid (22.6 mg, 37.85 μmol, 19.09% yield, 99% purity) as a white solid. LCMS; 591.31 [M+1].

### 13. Experimental Procedure for Compound 267

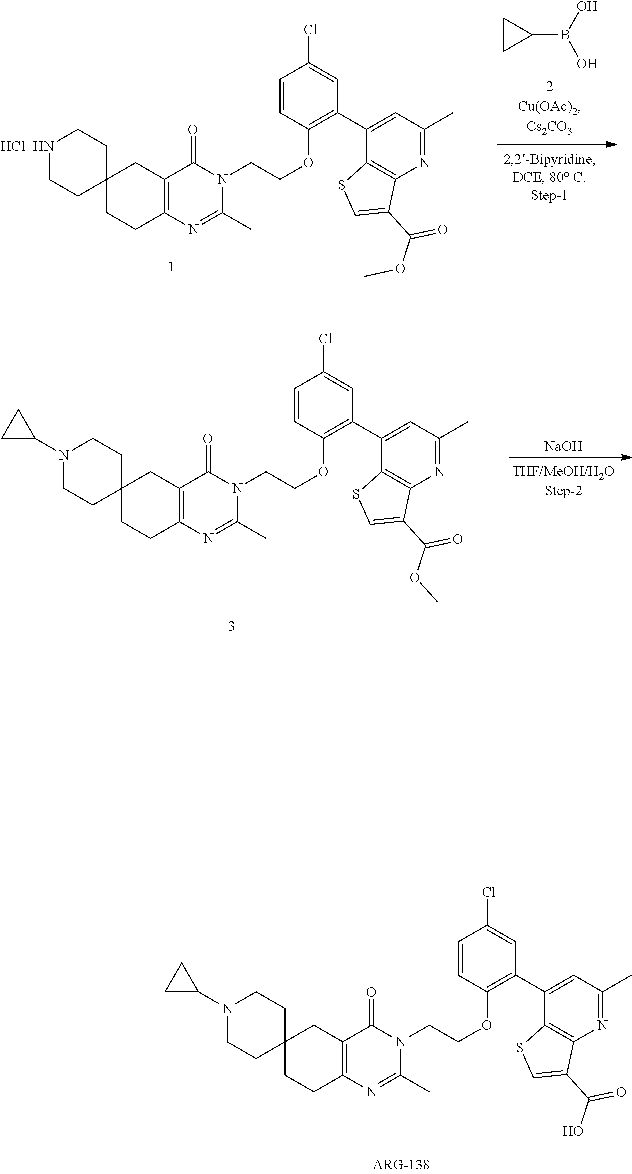 [SMILES: Cl ; COC(=O)c1csc2c(-c3cc(Cl)ccc3OCCn3c(C)nc4c(c3=O)CC3(CCNCC3)CC4)cc(C)nc12 ; OB(O)C1CC1 ; COC(=O)c1csc2c(-c3cc(Cl)ccc3OCCn3c(C)nc4c(c3=O)CC3(CC4)CCN(C4CC4)CC3)cc(C)nc12 ; "NaOH" ; Cc1cc(-c2cc(Cl)ccc2OCCn2c(C)nc3c(c2=O)CC2(CC3)CCN(C3CC3)CC2)c2scc(C(=O)O)c2n1]

### Step-1: Synthesis of methyl 7-(5-chloro-2-(2-(1-cyclopropyl-2′-methyl-4′-oxo-7′,8′-dihydro-4′H-spiro[piperidine-4,6′-quinazolin]-3′(5′H)-yl)ethoxy)phenyl)-5-methylthieno[3,2-b]pyridine-3-carboxylate (3)

To a stirred solution of methyl 7-[5-chloranyl-2-[2-(2-methyl-4-oxidanylidene-spiro[7,8-dihydro-5H-quinazoline-6,4′-piperidine]-3-yl)ethoxy]phenyl]-5-methyl-thieno[3,2-b]pyridine-3-carboxylate (100 mg, 158.83 μmol) in DCE (3 mL) were added cyclo-propylboronic acid (40.93 mg, 476.50 μmol), cesium carbonate (103.50 mg, 317.66 μmol), copper acetate (43.27 mg, 238.25 μmol), and 2,2 bipyridine (37.21 mg, 238.25 μmol). The resulting reaction mixture was stirred at 90° C. for 4 h. Progress of the reaction was monitored by LCMS. The reaction mixture was cooled to room temperature, poured into ice-cold water (5 mL) and stirred for 5 min at room temperature. The reaction mixture was then washed with 10 mL of Ethyl acetate. The combined organic layer was concentrated under reduced pressure to obtain the crude material. The reaction mixture was poured into hexanes (10 mL) and stirred for 10 min at room temperature. The resulting precipitate was filtered on Buchner funnel, washed with Hexanes (10 mL) and dried under reduced pressure to afford the desired product as a crude material methyl 7-[5-chloranyl-2-[2-(1′-cyclopropyl-2-methyl-4-oxidanylidene-spiro[7,8-dihydro-5H-quinazoline-6,4′-piperidine]-3-yl) ethoxy]phenyl]-5-methyl-thieno[3,2-b]pyridine-3-carboxylate (120 mg). LCMS: 633.22. [M+1].

### Step-2: Synthesis of 7-(5-chloro-2-(2-(1-cyclopropyl-2′-methyl-4′-oxo-7′,8′-dihydro-4′H-spiro[piperidine-4,6′-quinazolin]-3′(5′H)-yl)ethoxy)phenyl)-5-methylthieno[3,2-b]pyridine-3-carboxylic acid (Compound 267)

Sodium hydroxide (25.27 mg, 631.71 μmol, 11.86 μL) was added to the solution of methyl 7-[5-chloranyl-2-[2-(1′-cyclopropyl-2-methyl-4-oxidanylidene-spiro[7,8-dihydro-5H-quinazoline-6,4′-piperidine]-3-yl)ethoxy]phenyl]-5-methyl-thieno[3,2-b]pyridine-3-carboxylate (80 mg, 126.34 μmol) in a mixture of THF/methanol/H₂O (3 mL/1 mL/1 mL) at room temperature. The resulting mixture stirred at 50° C. for 1 h. Progress of the reaction was monitored by LCMS. The reaction mass was acidified with 1 M HCl/sat. citric acid solution to pH 4-6. The volatiles were removed under reduced pressure to obtain the crude material. The crude product was purified by prep-HPLC to afford 7-[5-chloranyl-2-[2-(1′-cyclopropyl-2-methyl-4-oxidanylidene-spiro[7,8-dihydro-5H-quinazoline-6,3′-azetidine]-3-yl)ethoxy]phenyl]-5-methyl-thieno[3,2-b]pyridine-3-carboxylic acid (22.6 mg, 37.85 μmol, 19.09% yield, 99% purity) as a white solid. LCMS: 619.22 [M+1].

### 14. Experimental Procedure for Compound 268

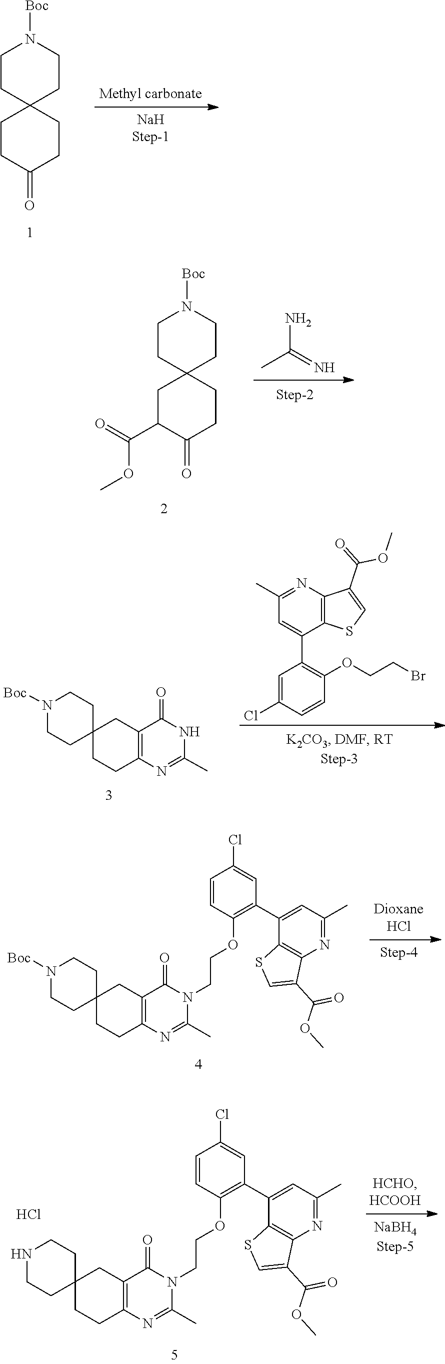

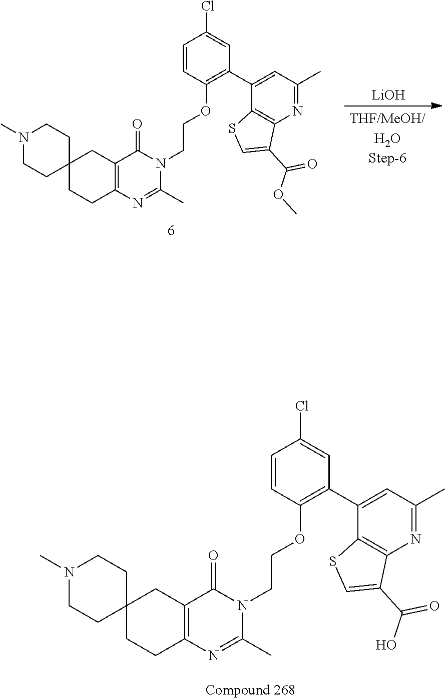

### Step-1: Synthesis of 3-(tert-butyl) 8-methyl 9-oxazaspiro[5.5]undecane-3,8-dicarboxylate (2)

To a stirred solution of sodium hydride 60% dispersion in mineral oil (4.30 g, 187.01 mmol) was added in dimethyl carbonate (16.85 g, 187.01 mmol, 15.74 mL) at 0-5° C. and the resulting reaction mixture was stirred at room temperature for 10 min. 1,1-di(methyl)ethyl 9-oxidanylidene-3-azaspiro[5.5]undecane-3-carboxylate (5.0 g, 18.70 mmol) in dimethyl carbonate (8.42 g, 93.51 mmol, 7.87 mL) was added drop wisely to the resulting reaction at 0-5° C. and stirring continued at 80° C. for 3 h under N₂ atmosphere. Progress of the reaction was monitored by LCMS. The reaction mixture was quenched with ice-cold water (50.0 mL) and washed with ethyl acetate (100 mL×2). The combined organic layer was washed with brine solution (30 mL×2), dried over Na₂SO₄ and filtered. The filtrate was concentrated under reduced pressure to obtained crude product. This was purified by flash column chromatography using 100-200 silica gel mesh and the desired product was eluted at 5% ethyl acetate in pet-ether as the eluent. Pure fractions (determined by TLC) were combined and evaporated under reduced pressure to afford 1,1-di(methyl)ethyl 2-methyl-4-oxidanylidene-spiro[3,5,7,8-tetrahydroquinazoline-6,4′-piperidine]-1′-carboxylate (1500 mg, 4.50 mmol, 33.27% yield). LCMS; 326.46 [M+1].

### Step-2: Synthesis of tert-butyl 2′-methyl-4′-oxo-3′,5′,7′,8′-tetrahydro-4′H-spiro[piperidine-4,6′-quinazoline]-1-carboxylate (3)

2 synthesized using general procedure (RBX-6262: Step-4) from 3-[1,1-di(methyl)ethyl]O10-methyl 9-oxidanylidene-3-azaspiro[5.5]undecane-3,10-dicarboxylate (4.4 g, 13.52 mmol) to afford 1,1-di(methyl)ethyl 2-methyl-4-oxidanylidene-spiro[3,5,7,8-tetrahydroquinazoline-6,4′-piperidine]-1′-carboxylate (1500 mg, 4.50 mmol, 33.27% yield) was obtained. LCMS; 334.15 [M+1].

### Step-3: Synthesis of methyl 7-(2-(2-(1-(tert-butoxycarbonyl)-2′-methyl-4′-oxo-7′,8′-dihydro-4′H-spiro[piperidine-4,6′-quinazolin]-3′(5′H)-yl)ethoxy)-5-chlorophenyl)-5-methylthieno[3,2-b]pyridine-3-carboxylate (4)

4 synthesized using general procedure (RBX-6262: Step-5) from 1,1-di(methyl)ethyl 2-methyl-4-oxidanylidene-spiro[3,5,7,8-tetrahydroquinazoline-6,4′-piperidine]-1′-carboxylate (1 g, 3.00 mmol) and methyl 7-[2-(2-bromanylethoxy)-5-chloranyl-phenyl]-5-methyl-thieno[3,2-b]pyridine-3-carboxylate (793.11 mg, 1.80 mmol) to afford methyl 7-[5-chloranyl-2-[2-[1′-[1,1-di(methyl)ethoxycarbonyl]-2-methyl-4-oxidanylidene-spiro[7,8-dihydro-5H-quinazoline-6,4′-piperidine]-3-yl]ethoxy]phenyl]-5-methyl-thieno[3,2-b]pyridine-3-carboxylate (400 mg, 300.04 mol, 10.00% yield, 52% purity) LCMS; 693.4 [M+1]

### Step-4: Synthesis of methyl 7-(5-chloro-2-(2-(2′-methyl-4′-oxo-7′,8′-dihydro-4′H-spiro[piperidine-4,6′-quinazolin]-3′(5′H)-yl)ethoxy)phenyl)-5-methylthieno[3,2-b]pyridine-3-carboxylate (5)

4 M HCl in Dioxane (1 mL) was added dropwise to an ice-cold solution of methyl 7-[5-chloranyl-2-[2-[1′-[1,1-di(methyl)ethoxycarbonyl]-2-methyl-4-oxidanylidene-spiro[7,8-dihydro-5H-quinazoline-6,4′-piperidine]-3-yl]ethoxy]phenyl]-5-methyl-thieno[3,2-b]pyridine-3-carboxylate (60 mg, 86.55 μmol) in DCM (2 mL) and the reaction mixture stirred for 25° C. for 1 h. Progress of the reaction was monitored by LCMS. The reaction mass was concentrated under reduced pressure to get crude material methyl 7-(5-chloro-2-(2-(2′-methyl-4′-oxo-7′,8′-dihydro-4′H-spiro[piperidine-4,6′-quinazolin]-3′(5′H)-yl)ethoxy)phenyl)-5-methylthieno[3,2-b]pyridine-3-carboxylate as a white solid. LCMS; 593.19 [M+1]

### Step-5: Synthesis of methyl 7-(5-chloro-2-(2-(1,2′-dimethyl-4′-oxo-7′,8′-dihydro-4′H-spiro[piperidine-4,6′-quinazolin]-3′(5′H)-yl)ethoxy)phenyl)-5-methylthieno[3,2-b]pyridine-3-carboxylate (6)

To a stirred solution of methyl 7-[5-chloranyl-2-[2-(2-methyl-4-oxidanylidene-spiro[7,8-dihydro-5H-quinazoline-6,4′-piperidine]-3-yl)ethoxy]phenyl]-5-methyl-thieno[3,2-b]pyridine-3-carboxylate (100 mg, 168.60 μmol) in Formaldehyde (37 wt % in water, 88.12 μL) and formic acid (352.48 μL). The reaction mixture was heated to 60° C. for 2 h. The reaction mixture was cooled to 0° C. Sodium Borohydride (63.78 mg, 1.69 mmol, 59.39 μL) was added to the resulting reaction mixture and stirring continued at 25° C. for 3 h. The progress of the reaction was monitored by LCMS. The reaction mixture was quenched by adding saturated NaHCO₃ solution (2 mL) and evaporated from the reaction mixture methyl 7-[5-chloranyl-2-[2-[1′,2-di(methyl)-4-oxidanylidene-spiro[7,8-dihydro-5H-quinazoline-6,4′-piperidine]-3-yl]ethoxy]phenyl]-5-methyl-thieno[3,2-b]pyridine-3-carboxylate (100 mg). LCMS: 607.60 [M+1].

### Step-6: Synthesis of 7-(5-chloro-2-(2-(1,2′-dimethyl-4′-oxo-7′,8′-dihydro-4′H-spiro[piperidine-4,6′-quinazolin]-3′(5′H)-yl)ethoxy)phenyl)-5-methylthieno[3,2-b]pyridine-3-carboxylic acid (Compound 268)

Sodium hydroxide (32.94 mg, 823.50 μmol, 15.46 μL) was added to the solution of methyl 7-[5-chloranyl-2-[2-[1′,2-di(methyl)-4-oxidanylidene-spiro[7,8-dihydro-5H-quinazoline-6,4′-piperidine]-3-yl]ethoxy]phenyl]-5-methyl-thieno[3,2-b]pyridine-2-carboxylate (100 mg, 164.70 μmol) in a mixture of THF/Methanol/H₂O (3 mL/1 mL/1 mL) at room temperature. The resulting mixture stirring continued at 50° C. for 1 h. Progress of the reaction was monitored by LCMS. The reaction mass was acidified with 1 M HCl/sat. citric acid solution to pH 4-6. The volatiles were removed under reduced pressure to obtain the crude material. The crude product was purified by prep-HPLC to afford 7-[5-chloranyl-2-[2-[1′,2-di(methyl)-4-oxidanylidene-spiro[7,8-dihydro-5H-quinazoline-6,4′-piperidine]-3-yl]ethoxy]phenyl]-5-methyl-thieno[3,2-b]pyridine-3-carboxylate (100 mg, 164.70 μmol) as a white solid. LCMS: 593.2[M+1].

### 15. Experimental Procedure for Compound 362, Compound 370

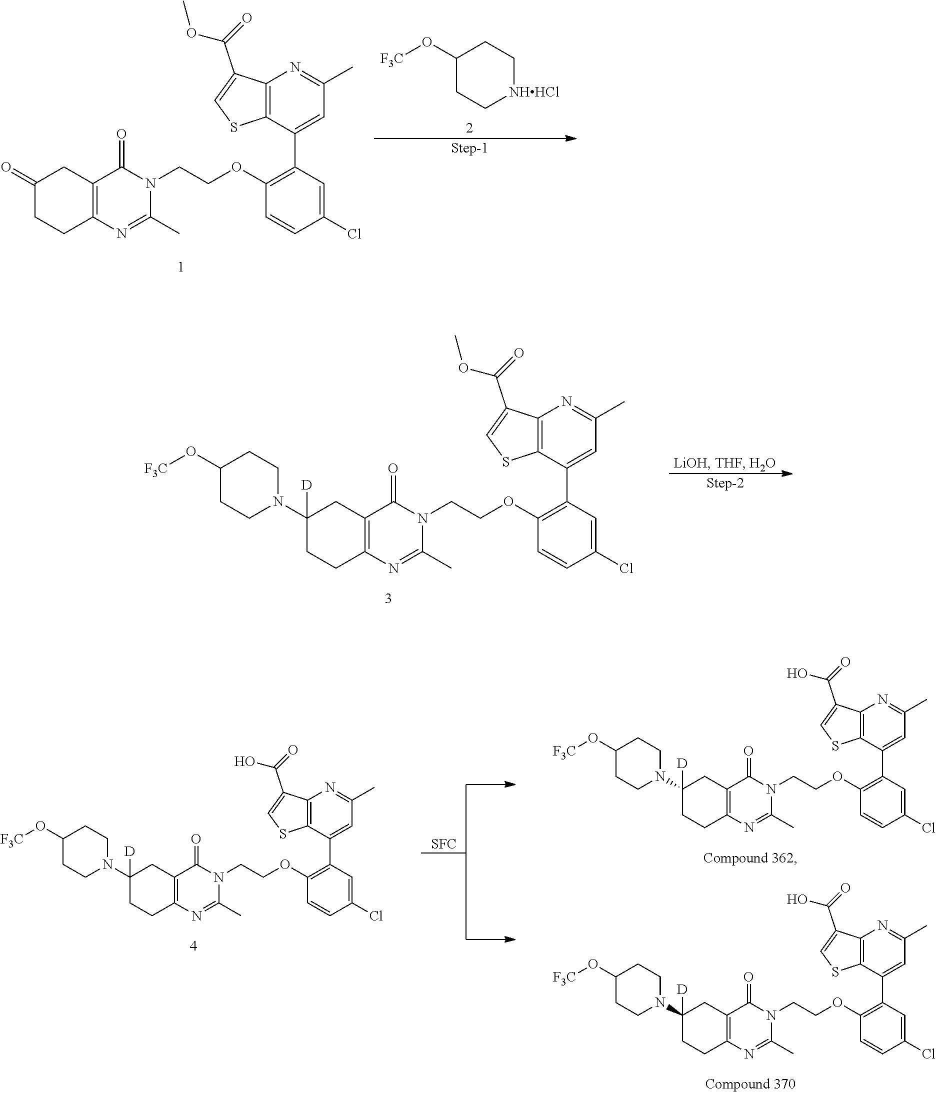

### Step 1: Synthesis of methyl 7-(5-chloro-2-(2-(2-methyl-4-oxo-6-(4-(trifluoromethoxy)piperidin-1-yl)-5,6,7,8-tetrahydroquinazolin-3(4H)-yl-6-d)ethoxy)phenyl)-5-methylthieno[3,2-b]pyridine-3-carboxylate (3)

To a stirred solution of 4-[tris(fluorenyl)methoxy]piperidine hydrochloride (286.62 mg, 1.39 mmol) in DCE (15 mL) was added TEA (141.06 mg, 1.39 mmol, 194.30 μL) at ambient temperature, then stirred for 10 minutes, followed by addition of methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (500 mg, 929.34 μmol). The reaction mixture was stirred for 12 h at ambient temperature. Then added sodium Borohydride-D4 (77.80 mg, 1.86 mmol) at 0° C. Reaction mixture was stirred for 2 h, reaction was monitored by LCMS. Reaction mixture was concentrated to get crude material compound. The crude material was purified by reverse phase flash chromatography (Biotage 12 g C₁₈ cartridge and compound eluted with a gradient of 0-100% 0.1 AA water in acetonitrile) to afford methyl 7-[5-chloro-2-[2-[6-deuterio-2-methyl-4-oxo-6-[4-(trifluoromethoxy)-1-piperidyl]-7,8-dihydro-5H-quinazolin-3-yl]ethoxy]phenyl]-5-methyl-thieno[3,2-b]pyridine-3-carboxylate (84 mg, 101.94 μmol, 10.97% yield, 84% purity) as off white solid. LCMS: 692.12 [M+1].

### Step-2: Synthesis of 7-(5-chloro-2-(2-(2-methyl-4-oxo-6-(4-(trifluoromethoxy)piperidin-1-yl)-5,6,7,8-tetrahydroquinazolin-3(4H)-yl-6-d)ethoxy)phenyl)-5-methylthieno[3,2-b]pyridine-3-carboxylic acid (4)

This compound was prepared using procedure B from methyl 7-[5-chloro-2-[2-[6-deuterio-2-methyl-4-oxo-6-[4-(trifluoromethoxy)-1-piperidyl]-7,8-dihydro-5H-quinazolin-3-yl]ethoxy]phenyl]-5-methyl-thieno[3,2-b]pyridine-3-carboxylate (78 mg, 112.69 μmol) afforded 7-(5-chloro-2-(2-(2-methyl-4-oxo-6-(4-(trifluoromethoxy)piperidin-1-yl)-5,6,7,8-tetrahydroquinazolin-3(4H)-yl-6-d)ethoxy)phenyl)-5-methylthieno[3,2-b]pyridine-3-carboxylic acid. This was purified SFC to obtain each isomers. ARG-662, (S)-7-(5-chloro-2-(2-(2-methyl-4-oxo-6-(4-(trifluoromethoxy)piperidin-1-yl)-5,6,7,8-tetrahydroquinazolin-3(4H)-yl-6-d)ethoxy)phenyl)-5-methylthieno[3,2-b]pyridine-3-carboxylic acid, LCMS: 678.25 [M+1]. ARG-663, (R)-7-(5-chloro-2-(2-(2-methyl-4-oxo-6-(4-(trifluoromethoxy)piperidin-1-yl)-5,6,7,8-tetrahydroquinazolin-3(4H)-yl-6-d)ethoxy)phenyl)-5-methylthieno[3,2-b]pyridine-3-carboxylic acid. LCMS: 678.25[M+1].

### 16. Experimental Procedure for Compound 598, Compound 597

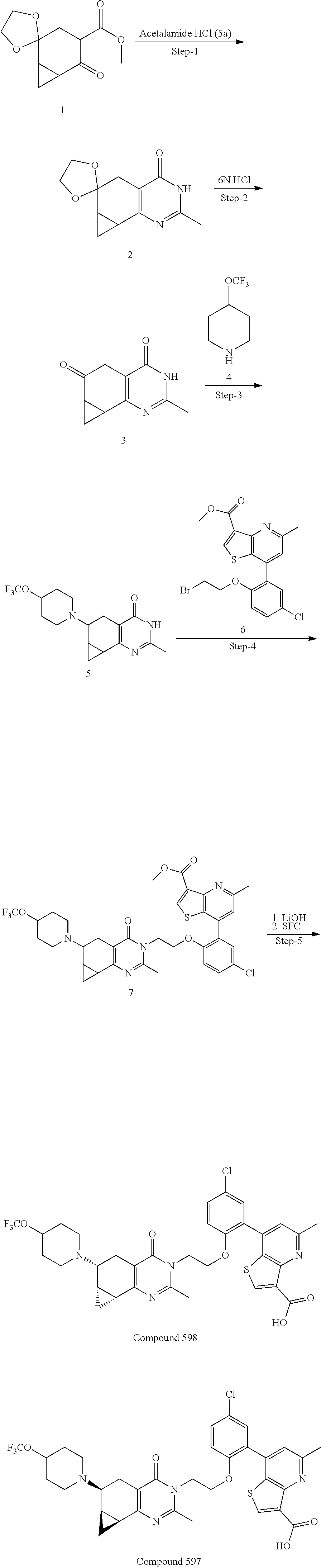

### Step-1: Synthesis of 2-methyl-5,6a, 7,7a-tetrahydrospiro[cyclopropa[h]quinazoline-6,2′-[1,3]dioxolan]-4(3H)-one (2)

Note: SM-1 is synthesized using procedure mentioned in WO2014018350 A1. Acetamidine hydrochloride (313.44 mg, 3.32 mmol) was added to the methyl 2′-oxidanylidenespiro[1,3-dioxolane-2,5′-norcarane]-3′-carboxylate (300 mg, 1.33 mmol) in methanol (1.5 mL) at room temperature. The reaction mixture was stirred for 15 minutes then sodium methoxide in 30% methanol (1.5 mL) was added and resulting reaction mixture heat to 80° C. The reaction mass was stirred at 80° C. for 28 h. After completion of reaction, reaction mass was washed by ethyl acetate. Collected organics, dried over sodium sulfate and concentrated under reduced pressure to afford 2-methyl-5,6a,7,7a-tetrahydrospiro[cyclopropa[h]quinazoline-6,2′-[1,3]dioxolan]-4(3H)-one. LCMS: 331.35 [M+1].

### Step-2: Synthesis of 2-methyl-5,6a, 7,7a-tetrahydro-3H-cyclopropa[h]quinazoline-4,6-dione (3)

To a stirred solution of 6′-methylspiro[1,3-dioxolane-2,2′-1a,3,5,7b-tetrahydro-1H-cyclopropa[h]quinazoline]-4′-one (160 mg, 683.03 μmol) in THF (2 mL) was added 6N HCl (0.2 mL) and reaction mixture at was stirred at room temperature for 6 h. After completion of the reaction, reaction mass was brought to pH ˜8 by slowly adding 10% NaHCO₃ solution. This reaction mass was washed with 10% Methanol in DCM. Collected organics, dried over sodium sulfate and concentrated under reduced pressure to get 6-methyl-1a,3,5,7b-tetrahydro-1H-cyclopropa[h]quinazoline-2,4-dione (140 mg, 478.45 μmol, 70.05% yield, 65% purity). LCMS 190.86 [M+1].

### Step-3: Synthesis of 2-methyl-6-(4-(trifluoromethoxy)piperidin-1-yl)-3,5,6,6a, 7,7a-hexahydro-4H-cyclopropa[h]quinazolin-4-one (5)

To a stirred solution of 4-(trifluoromethoxy)piperidine (16.01 mg, 94.64 μmol) in DCE (1.95 mL) was added titanium isopropoxide (67.24 mg, 236.60 μmol, 70.41 μL) at room temperature for 10 minutes followed by addition of 6-methyl-1a,3,5,7b-tetrahydro-1H-cyclopropa[h]quinazoline-2,4-dione (15 mg, 78.87 μmol) at room temperature. After that TEA (11.97 mg, 118.30 μmol, 16.49 μL) was added to above mixture and it was stirred for 16 h. After that, sodium cyanoborohydride (14.87 mg, 236.60 μmol) was added and reaction mixture stirred at room temperature for 16 h. After completion of reaction, water (20 mL) was added and washed with 20% IPA in chloroform (2×30 mL). The combined organic layer was dried over Na₂SO₄ and filtered. The filtrate was concentrated under reduced pressure to obtained crude product. This was purified by flash column chromatography using 100-200 mesh silica gel and the desired product was eluted at 10-15% of Methanol in DCM as the eluent. Pure fractions (determined by TLC) were combined and evaporated under reduced pressure to afford 6-methyl-2-[4-[tris(fluorenyl)methoxy]-1-piperidyl]-1,1a,2,3,5,7b-hexahydrocyclopropa[h]quinazolin-4-one. LCMS: 687.37 [M+1].

### Step-4: Synthesis of methyl 7-(5-chloro-2-(2-(2-methyl-4-oxo-6-(4-(trifluoromethoxy)piperidin-1-yl)-4,5,6,6a, 7,7a-hexahydro-3H-cyclopropa[h]quinazolin-3-yl)ethoxy)phenyl)-5-methylthieno[3,2-b]pyridine-3-carboxylate (7)

Potassium carbonate (60.38 mg, 436.88 μmol, 26.37 μL) was added to the 6-methyl-2-[4-[tris(fluorenyl)methoxy]-1-piperidyl]-1,1a,2,3,5,7b-hexahydrocyclopropa[h]quinazolin-4-one (50 mg, 145.63 μmol) in DMF (478.91 μL) at room temperature and reaction mixture was stirred for 15 minutes. After that, methyl 7-(2-(2-bromoethoxy)-5-chlorophenyl)-5-methylthieno[3,2-b]pyridine-3-carboxylate (64.18 mg, 145.63 μmol) was added, and reaction mixture was heated at 80° C. for 28 h. After completion of reaction, excess solvent was evaporated to obtain methyl 7-[5-chloranyl-2-[2-[6-methyl-4-oxidanylidene-2-[4-[tris(fluorenyl)methoxy]-1-piperidyl]-1a,2,3,7b-tetrahydro-1H-cyclopropa[h]quinazolin-5-yl]ethoxy]phenyl]-5-methyl-thieno[3,2-b]pyridine-3-carboxylate (200 mg, crude material). LCMS 703.29 [M+1].

### Step-5: Synthesis of (5-chloro-2-(2-((6R,6aS,7aR)-2-methyl-4-oxo-6-(4-(trifluoromethoxy)piperidin-1-yl)-4,5,6,6a,7,7a-hexahydro-3H-cyclopropa[h]quinazolin-3-yl)ethoxy)phenyl)-5-methylthieno[3,2-b]pyridine-3-carboxylic acid, 7-(5-chloro-2-(2-((6S,6aR,7aS)-2-methyl-4-oxo-6-(4-(trifluoromethoxy)piperidin-1-yl)-4,5,6,6a,7,7a-hexahydro-3H-cyclopropa[h]quinazolin-3-yl)ethoxy)phenyl)-5-methylthieno[3,2-b]pyridine-3-carboxylic acid, 7-(5-chloro-2-(2-(((6R,6aS,7aR)-2-methyl-6-(4-(trifluoromethoxy)piperidin-1-yl)-6,6a,7,7a-tetrahydro-5H-cyclopropa[h]quinazolin-4-yl)oxy)ethoxy)phenyl)-5-methylthieno[3,2-b]pyridine-3-carboxylic acid and 7-(5-chloro-2-(2-(((6S,6aR,7aS)-2-methyl-6-(4-(trifluoromethoxy)piperidin-1-yl)-6,6a,7,7a-tetrahydro-5H-cyclopropa[h]quinazolin-4-yl)oxy)ethoxy)phenyl)-5-methylthieno[3,2-b]pyridine-3-carboxylic acid (Compound 598, Compound 597)

These examples were synthesized using procedure B using methyl 7-[5-chloranyl-2-[2-[6-methyl-4-oxidanylidene-2-[4-[tris(fluorenyl)methoxy]-1-piperidyl]-1a,2,3,7b-tetrahydro-1H-cyclopropa[h]quinazolin-5-yl]ethoxy]phenyl]-5-methyl-thieno[3,2-b]pyridine-3-carboxylate (200 mg, 284.43 μmol) afforded (ARG-1034) 7-(5-chloro-2-(2-((6R,6aS,7aR)-2-methyl-4-oxo-6-(4-(trifluoromethoxy)piperidin-1-yl)-4,5,6,6a,7,7a-hexahydro-3H-cyclopropa[h]quinazolin-3-yl)ethoxy)phenyl)-5-methylthieno[3,2-b]pyridine-3-carboxylic acid LCMS: 689.29 [M+1]. ARG-1035, 7-(5-chloro-2-(2-((6S,6aR,7aS)-2-methyl-4-oxo-6-(4-(trifluoromethoxy)piperidin-1-yl)-4,5,6,6a,7,7a-hexahydro-3H-cyclopropa[h]quinazolin-3-yl)ethoxy)phenyl)-5-methylthieno[3,2-b]pyridine-3-carboxylic acid, LCMS: 689.22 [M+1]. ARG-1036, 7-(5-chloro-2-(2-(((6R,6aS,7aR)-2-methyl-6-(4-(trifluoromethoxy)piperidin-1-yl)-6,6a,7,7a-tetrahydro-5H-cyclopropa[h]quinazolin-4-yl)oxy)ethoxy)phenyl)-5-methylthieno[3,2-b]pyridine-3-carboxylic acid, LCMS: 689.25 [M+1]. ARG-1037, 7-(5-chloro-2-(2-(((6S,6aR,7aS)-2-methyl-6-(4-(trifluoromethoxy)piperidin-1-yl)-6,6a,7,7a-tetrahydro-5H-cyclopropa[h]quinazolin-4-yl)oxy)ethoxy)phenyl)-5-methylthieno[3,2-b]pyridine-3-carboxylic acid, LCMS 689.26 [M+1].

### 17. Experimental Procedure for Compound 631 and Compound 627

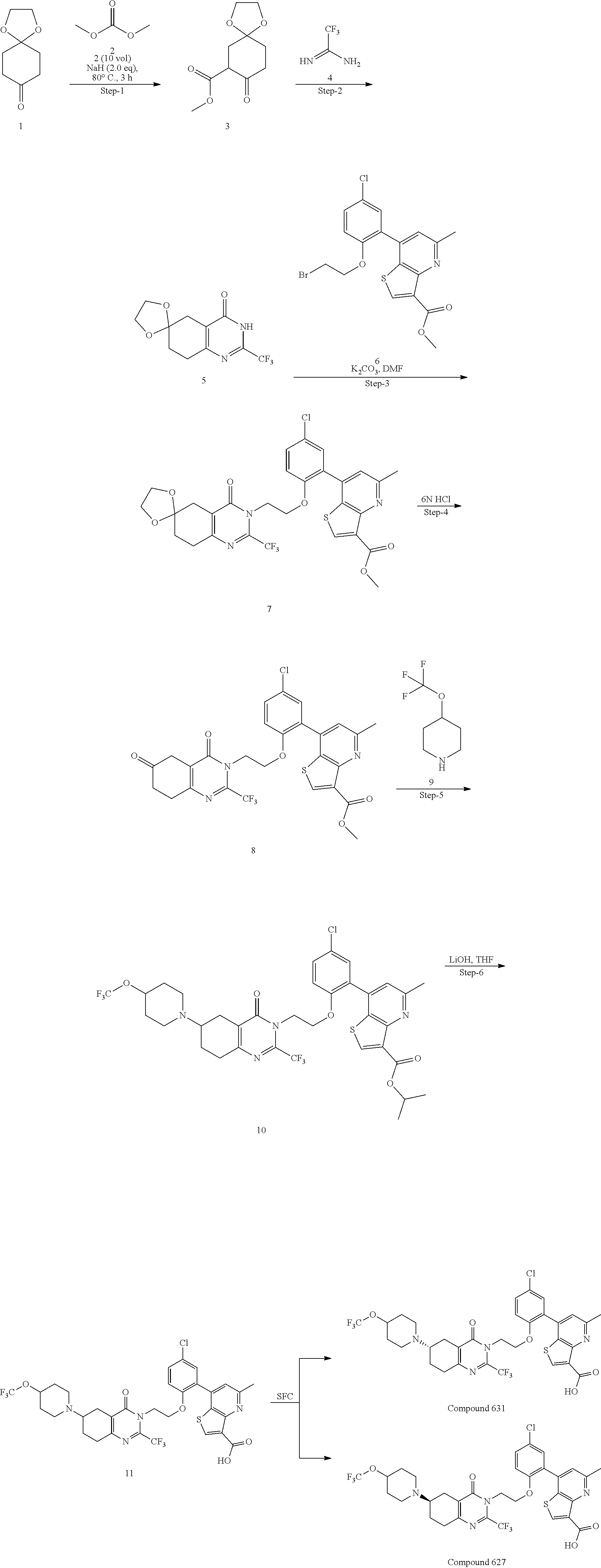

### Step-1: Synthesis of methyl 8-oxo-1,4-dioxaspiro[4.5]decane-7-carboxylate (3)

To a stirred solution of sodium hydride (1.54 g, 64.03 mmol) was added in dimethyl carbonate (2.88 g, 32.01 mmol, 2.62 mL) at 0° C. The mixture was stirred at room temperature for 5 minutes and then added 1,4-dioxaspiro[4.5]decan-8-one (5 g, 32.01 mmol, dissolved in dimethyl carbonate). The reaction mixture was stirred at 80° C. for 3 h. The progress of reaction was monitored by LCMS and TLC. After completion of the reaction, the reaction mixture was quenched with ammonium chloride solution at 0° C. and washed with ethyl acetate. The organic layer was washed with brine and dried over Na₂SO₄ and concentrated in vacuo. The obtained crude material was purified by flash column chromatography by using 230-400 silica gel by using eluent 10-30% (Ethyl acetate:Hexane) to obtain methyl 8-oxidanylidene-1,4-dioxaspiro[4.5]decane-6-carboxylate (2.5 g, 36.45% yield) LCMS; 215.03 [M+1].

### Step-2: Synthesis of 2-(trifluoromethyl)-3,5,7,8-tetrahydro-4H-spiro[quinazoline-6,2′-[1,3]dioxolan]-4-one (6)

To the stirred solution of methyl 8-oxidanylidene-1,4-dioxaspiro[4.5]decane-7-carboxylate (0.7 g, 3.27 mmol) in Methanol (5 mL) was added sodium methoxide (25% in methanol, 882.68 mg, 16.34 mmol, 910.92 μL) followed by the addition of 2,2,2-tris(fluorenyl)acetamidine (549.25 mg, 4.90 mmol, 367.63 μL). The mixture was allowed to stir at 80° C. for 1 h. After completion of the reaction, the volatiles were removed in vacuo. The obtained crude material was treated with saturated citric acid aqueous solution to neutralize the excess base (pH˜7) and washed with 10% Methanol-DCM (3×30 ml). The organic layer was concentrated to dryness and obtained solid was used directly to next step. (0.75 g, 83.09% yield) LCMS; 277.11 [M+1].

### Step-3: Synthesis of methyl 7-(5-chloro-2-(2-(4-oxo-2-(trifluoromethyl)-7,8-dihydro-4H spiro[quinazoline-6,2′-[1,3]dioxolan]-3(5H)-yl)ethoxy)phenyl)-5-methylthieno[3,2-b]pyridine-3-carboxylate (7)

To a stirred solution of 2′-(trifluoromethyl)spiro[1,3-dioxolane-2,6′-3,5,7,8-tetrahydroquinazoline]-4′-one (1 g, 3.62 mmol) and methyl 7-[2-(2-bromoethoxy)-5-chloro-phenyl]-5-methyl-thieno[3,2-b]pyridine-3-carboxylate (1.91 g, 4.34 mmol) in N,N′-dimethyl formamide (10 mL) was added potassium carbonate (1.75 g, 12.67 mmol) and stirred the reaction mixture at 80° C. for 16 h. After completion, the reaction mixture was poured into ice-cold water and the obtained solid was filtered. The obtained crude material was purified by reverse phase column chromatography using ACN-water as eluent to afford methyl 7-(5-chloro-2-(2-(4-oxo-2-(trifluoromethyl)-7,8-dihydro-4H-spiro[quinazoline-6,2′-[1,3]dioxolan]-3(5H)-yl)ethoxy)phenyl)-5-methylthieno[3,2-b]pyridine-3-carboxylate (0.310 g, 14% yield). LCMS: 636.07 [M+1].

### Step-4: Synthesis of methyl 7-(5-chloro-2-(2-(4,6-dioxo-2-(trifluoromethyl)-5,6,7,8-tetrahydroquinazolin-3(4H)-yl)ethoxy)phenyl)-5-methylthieno[3,2-b]pyridine-3-carboxylate (8)

To stirred solution of methyl 7-[5-chloro-2-[2-[4′-oxo-2′-(trifluoromethyl)spiro[1,3-dioxolane-2,6′-7,8-dihydro-5H-quinazoline]-3′-yl]ethoxy]phenyl]-5-methyl-thieno[3,2-b]pyridine-3-carboxylate (600 mg, 943.34 μmol) in THF (10 mL) were added 6M HCl (12 mL) at room temperature. The resulting reaction mixture was stirred at 70° C. for 4 h. After completion of the reaction, reaction was quenched with saturated sodium bicarbonate aqueous solution (50 mL) and washed with 10% Methanol:DCM (3×100 mL). The combined organic layers were dried over Na₂SO₄, filtered and concentrated to afford methyl 7-[5-chloro-2-[2-[4,6-dioxo-2-(trifluoromethyl)-7,8-dihydro-5H-quinazolin-3-yl]ethoxy]phenyl]-5-methyl-thieno[3,2-b]pyridine-3-carboxylate (0.51 g, 89.53% yield). LCMS: 592.07 [M+1].

### Step-5: Synthesis of isopropyl 7-(5-chloro-2-(2-(4-oxo-6-(4-(trifluoromethoxy)piperidin-1-yl)-2-(trifluoromethyl)-5,6,7,8-tetrahydroquinazolin-3(4H)-yl)ethoxy)phenyl)-5-methylthieno[3,2-b]pyridine-3-carboxylate (10)

Titanium isopropoxide (600.13 mg, 2.11 mmol, 631.72 μL) was added to a mixture of methyl 7-[5-chloro-2-[2-[4,6-dioxo-2-(trifluoromethyl)-7,8-dihydro-5H-quinazolin-3-yl]ethoxy]phenyl]-5-methyl-thieno[3,2-b]pyridine-3-carboxylate (250 mg, 422.31 μmol) and 4-(trifluoromethoxy)piperidine (107.15 mg, 633.46 μmol) was added triethylamine (64.10 mg, 633.46 μmol, 87.81 μL) in 1,2-dichloroethane (8 mL) at 27° C. The resulting reaction mixture was stirred at 70° C. for 16 h. It was then cooled to 27° C. and sodium cyanoborohydride (79.62 mg, 1.27 mmol) was added in portions. The resulting reaction mixture was stirred for 2 h. The reaction mixture was poured into ice-cold water (25 mL) and filtered through a celite pad and washed with 10% Methanol:DCM (50 mL). The organic layer was separated, and the aqueous layer was washed again with 10% Methanol:DCM (50 mL). The combined organic layer was concentrated under reduced pressure to afford isopropyl 7-(5-chloro-2-(2-(4-oxo-6-(4-(trifluoromethoxy)piperidin-1-yl)-2-(trifluoromethyl)-5,6,7,8-tetrahydroquinazolin-3(4H)-yl)ethoxy)phenyl)-5-methylthieno[3,2-b]pyridine-3-carboxylate (0.21 g, 61% yield) LCMS: 773.19 [M+1].

### Step-6: Synthesis of 7-(5-chloro-2-(2-(4-oxo-6-(4-(trifluoromethoxy)piperidin-1-yl)-2-(trifluoromethyl)-5,6,7,8-tetrahydroquinazolin-3(4H)-yl)ethoxy)phenyl)-5-methylthieno[3,2-b]pyridine-3-carboxylic acid (Compound 631 and Compound 627)

To stirred solution of isopropyl 7-[5-chloro-2-[2-[4-oxo-6-[4-(trifluoromethoxy)-1-piperidyl]-2-(trifluoromethyl)-5,6,7,8-tetrahydroquinazolin-3-yl]ethoxy]phenyl]-5-methyl-thieno[3,2-b]pyridine-3-carboxylate (0.1 g, 129.34 μmol) in THF (5 mL) and water (2 mL) was added lithium hydroxide monohydrate, 98% (27.14 mg, 646.68 μmol, 17.97 μL) at 27° C., and the reaction mixture was stirred at 27° C. for 4 h. The progress of the reaction was monitored by LCMS. After the completion, the volatiles were evaporated under reduced pressure and the obtained crude material was acidified by 1N HCl aqueous solution and washed with 10% Methanol-DCM. The organic layer was dried over Na₂SO₄ and concentrated in vacuo. The obtained crude material compound was purified by prep-HPLC and isomer separation were carried by SFC-purification method to afford 7-[5-chloro-2-[2-[(6S)-4-oxo-6-[4-(trifluoromethoxy)-1-piperidyl]-2-(trifluoromethyl)-5,6,7,8-tetrahydroquinazolin-3-yl]ethoxy]phenyl]-5-methyl-thieno[3,2-b]pyridine-3-carboxylic acid (20 mg, 26.90 μmol, 20.80% yield, 98.35% purity) and 7-[5-chloro-2-[2-[(6R)-4-oxo-6-[4-(trifluoromethoxy)-1-piperidyl]-2-(trifluoromethyl)-5,6,7,8-tetrahydroquinazolin-3-yl]ethoxy]phenyl]-5-methyl-thieno[3,2-b]pyridine-3-carboxylic acid (21 mg, 28.55 μmol, 22.07% yield, 99.38% purity).

### 18. Experimental Procedure for Compound 244

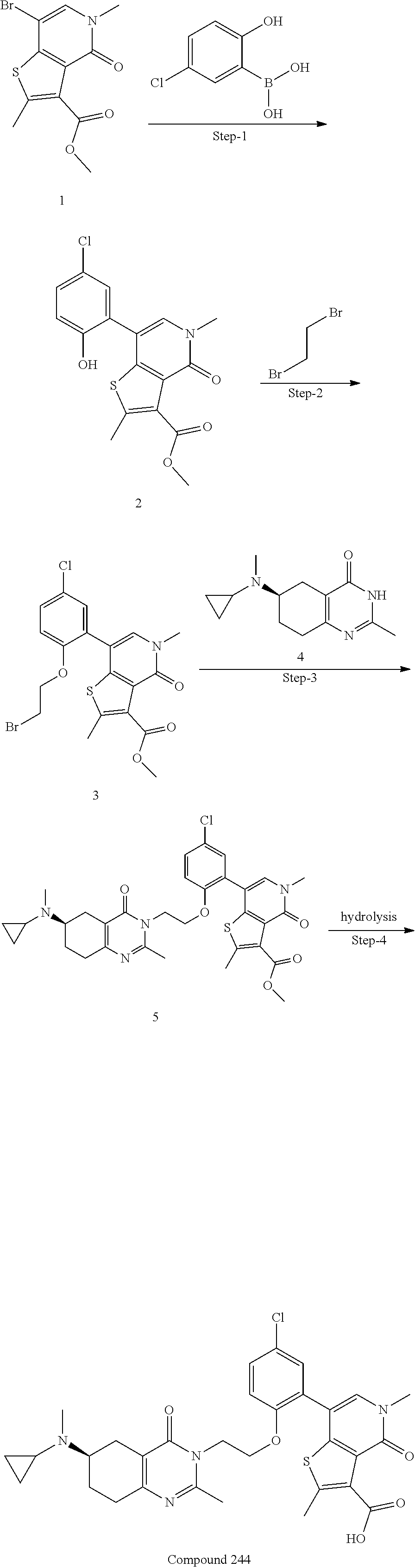

### Step-1: Synthesis of methyl 7-(5-chloro-2-hydroxyphenyl)-2,5-dimethyl-4-oxo-4,5-dihydrothieno[3,2-c]pyridine-3-carboxylate (2)

To a stirred solution of methyl 7-bromanyl-2,5-di(methyl)-4-oxidanylidene-thieno[3,2-c]pyridine-3-carboxylate (450 mg, 1.42 mmol) and (5-chloranyl-2-oxidanyl-phenyl)boronic acid (318.94 mg, 1.85 mmol) in 1,4-Dioxan (23 mL) at 25° C. was added potassium phosphate (604.23 mg, 2.85 mmol, dissolved in water). The resulting mixture was degassed by using Nitrogen gas for 10 minutes, then [1,1′-Bis(diphenylphosphino)ferrocene]dichloropalladium(II) (103.90 mg, 142.13 μmol) was added and again degassed for 5 minutes. The reaction mixture was stirred at 90° C. for 2 h. The progress of the reaction was monitored by TLC and LC-MS. The reaction mixture was diluted with water and washed with ethyl acetate (2×20 mL). The combined organic layer was dried over anhydrous Na₂SO₄, filtered and concentrated to get crude product. The crude product was purified by using water (20 V) and ethyl acetate (10 V), solid was filtered and dried under vacuum to afford methyl 7-(5-chloranyl-2-oxidanyl-phenyl)-2,5-di(methyl)-4-oxidanylidene-thieno[3,2-c]pyridine-3-carboxylate (300 mg, 799.86 μmol, 56.20% yield, 97% purity) as a brown colour solid. LCMS: 364.4

### Step-2: Synthesis of methyl 7-(2-(2-bromoethoxy)-5-chlorophenyl)-2,5-dimethyl-4-oxo-4,5-dihydrothieno[3,2-c]pyridine-3-carboxylate (3)

To a stirred solution of methyl 7-(5-chloranyl-2-oxidanyl-phenyl)-2,5-di(methyl)-4-oxidanylidene-thieno[3,2-c]pyridine-3-carboxylate (300 mg, 824.60 μmol) in ACN (6 mL) at 25° C. was added potassium carbonate, anhydrous, 99% (227.93 mg, 1.65 mmol, 99.53 μL) followed by 1,2-dibromoethane (1.55 g, 8.25 mmol, 710.59 μL). The resulting mixture was stirred at 90° C. for 16 h. The progress of the reaction was monitored by TLC. The reaction mixture was diluted with ethyl acetate (2×50 mL) and filtered through celite pad and filtrate was concentrated to get a crude product methyl 7-[2-(2-bromanylethoxy)-5-chloranyl-phenyl]-2,5-di(methyl)-4-oxidanylidene-thieno[3,2-c]pyridine-3-carboxylate (350 mg, 646.82 μmol, 78.44% yield, 87% purity) as a brown colour solid. The crude product was used to next step without purification.

### Step-3: Synthesis of methyl (R)-7-(5-chloro-2-(2-(6-(cyclopropyl(methyl)amino)-2-methyl-4-oxo-5,6,7,8-tetrahydroquinazolin-3(4H)-yl)ethoxy)phenyl)-2,5-dimethyl-4-oxo-4,5-dihydrothieno[3,2-c]pyridine-3-carboxylate (5)

To a stirred solution of 6-(((3,3-difluorocyclobutyl)methyl)(methyl)amino)-2-methyl-5,6,7,8-tetrahydroquinazolin-4(3H)-one (10 mg, 33.63 μmol) in DMF (0.5 mL) at ambient temperature were added methyl 7-(2-(2-bromoethoxy)-5-chlorophenyl)-2,5-dimethyl-4,5-dihydrothieno[3,2-c]pyridine-3-carboxylate (15.36 mg, 33.63 μmol) and Cerium (III) Carbonate (15.48 mg, 33.63 μmol). The resulting mixture was stirred at 80° C. for 3 h. The progress of the reaction was monitored by TLC. The reaction mixture was cooled to room temperature and added water into the reaction mixture, the solid was filtered and dried under vacuum to get a crude product. The crude product was purified by reverse phase column chromatography by using 0.01% ABC in water and ACN as a eluent and the required pure fractions were collected and concentrated to afford a desired product methyl 7-(5-chloro-2-(3-(6-(((3,3-difluorocyclobutyl)methyl)(methyl)amino)-2-methyl-4-oxo-5,6,7,8-tetrahydroquinazolin-3(4H)-yl)propoxy)phenyl)-2,5-dimethyl-4,5-dihydrothieno[3,2-c]pyridine-3-carboxylate (5 mg, 2.11 mol, 6.28% yield, 29.01% purity) as an off-white solid.

### Step-4: Synthesis of (R)-7-(5-chloro-2-(2-(6-(cyclopropyl(methyl)amino)-2-methyl-4-oxo-5,6,7,8-tetrahydroquinazolin-3(4H)-yl)ethoxy)phenyl)-2,5-dimethyl-4-oxo-4,5-dihydrothieno[3,2-c]pyridine-3-carboxylic acid (Compound 244)

To a stirred solution of methyl 7-[5-chloranyl-2-[2-[6-[cyclopropyl(methyl)amino]-2-methyl-4-oxidanylidene-5,6,7,8-tetrahydroquinazolin-3-yl]ethoxy]phenyl]-2,5-di(methyl)-4-oxidanylidene-thieno[3,2-c]pyridine-3-carboxylate (131.99 mg, 211.81 μmol) in THF (2 mL) and Methanol (0.5 mL) the reaction mixture was cooling condition was added in Lithium hydroxide monohydrate, 98% (53.33 mg, 1.27 mmol, 35.32 μL) in water (1 mL) after that the reaction mixture was stirred at room temperature for 16 hrs. The progress of the reaction was monitored by LCMS. The reaction mixture was concentrated until removal of THE then acidified with 1N HCl, under vacuum to obtain crude product as an off-white solid. The crude product was purified by reverse phase column chromatography by using 0.01% ABC in Water and ACN as a eluent followed by SFC separation to afford a desired product 7-[5-chloranyl-2-[2-[(6S)-6-[cyclopropyl(methyl)amino]-2-methyl-4-oxo-5,6,7,8-tetrahydroquinazolin-3-yl]ethoxy]phenyl]-2,5-di(methyl)-4-oxidanylidene-thieno[3,2-c]pyridine-3-carboxylic acid (5.13 mg, 8.40 μmol, 3.97% yield, 99.75% purity). The crude product was purified by SFC prep-HPLC purification and separated two peaks by using 0.5% DEA in methanol as an eluent. The required two pure fractions were collected and concentrated to afford peak-1: 7-[5-chloranyl-2-[2-[(6S)-6-[(3,3-difluorocyclobutyl)methyl-methyl-amino]-2-methyl-4-oxo-5,6,7,8-tetrahydroquinazolin-3-yl]ethoxy]phenyl]-2,5-di(methyl)-4-oxidanylidene-thieno[3,2-c]pyridine-3-carboxylic acid (25 mg, 36.66 μmol, 16.79% yield, 98.7% purity) and 7-[5-chloranyl-2-[2-[(6R)-6-[(3,3-difluorocyclobutyl)methyl-methyl-amino]-2-methyl-4-oxo-5,6,7,8-tetrahydroquinazolin-3-yl]ethoxy]phenyl]-2,5-di(methyl)-4-oxidanylidene-thieno[3,2-c]pyridine-3-carboxylic acid (22 mg, 31.63 μmol, 14.49% yield, 96.77% purity) as an off-white solid. LCMS: 609.2 [M+1].

### 19. Experimental Procedure for Compound 201

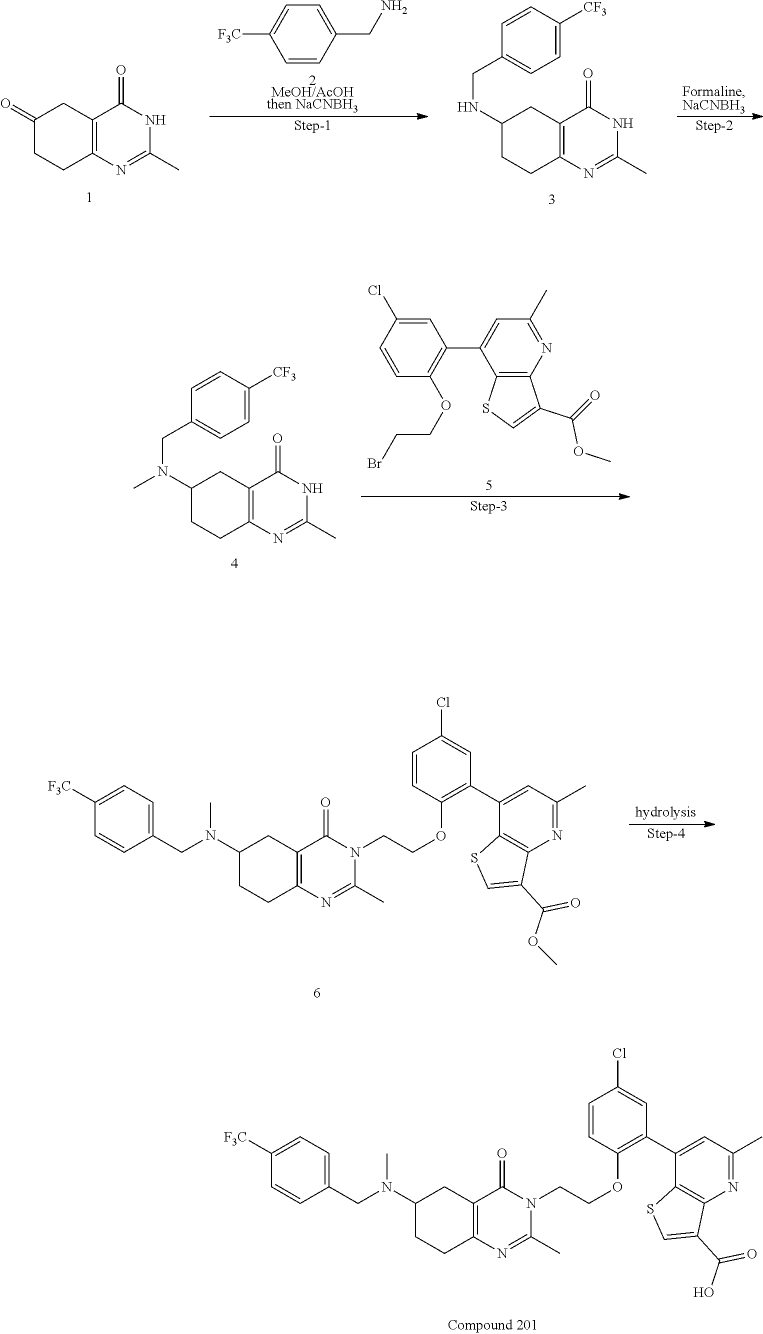

### Step-1: Synthesis of 2-methyl-6-((4-(trifluoromethyl)benzyl)amino)-5,6,7,8-tetrahydroquinazolin-4(3H)-one (3)

This intermediate was prepared by procedure A8 from 2-methyl-3,5,7,8-tetrahydroquinazoline-4,6-dione (3 g, 16.84 mmol) and [4-[tris(fluorenyl)methyl]phenyl]methanamine (3.54 g, 20.20 mmol, 2.88 mL) to afford 2-methyl-6-[[4-[tris(fluorenyl)methyl]phenyl]methylamino]-5,6,7,8-tetrahydro-3H-quinazolin-4-one (2 g, crude) as an off white solid. LCMS (ESI): 338.2 [M+1].

### Step-2: Synthesis of 2-methyl-6-(methyl(4-(trifluoromethyl)benzyl)amino)-5,6,7,8-tetrahydroquinazolin-4(3H)-one (4)

This intermediate was prepared by procedure G from 2-methyl-6-[[4-[tris(fluorenyl)methyl]phenyl]methylamino]-5,6,7,8-tetrahydro-3H-quinazolin-4-one (2 g, 5.93 mmol) afforded 2-methyl-6-[methyl-[[4-[tris(fluorenyl)methyl]phenyl]methyl]amino]-5,6,7,8-tetrahydro-3H-quinazolin-4-one (600 mg, 1.11 mmol, 18.72% yield, 65% purity) as an off white solid. LCMS: 352.2 [M+1].

### Step-3: Synthesis of methyl 7-(5-chloro-2-(2-(2-methyl-6-(methyl(4-(trifluoromethyl)benzyl)amino)-4-oxo-5,6,7,8-tetrahydroquinazolin-3(4H)-yl)ethoxy)phenyl)-5-methylthieno[3,2-b]pyridine-3-carboxylate (6)

This intermediate was prepared by procedure B from 2-methyl-6-[methyl-[[4-[tris(fluorenyl)methyl]phenyl]methyl]amino]-5,6,7,8-tetrahydro-3H-quinazolin-4-one (0.6 g, 1.71 mmol) and methyl 7-[2-(2-bromanylethoxy)-5-chloranyl-phenyl]-5-methyl-thieno[3,2-b]pyridine-3-carboxylate (451.57 mg, 1.02 mmol) afforded methyl 7-[5-chloranyl-2-[2-[2-methyl-6-[methyl-[[4-[tris(fluorenyl)methyl]phenyl]methyl]amino]-4-oxidanylidene-5,6,7,8-tetrahydroquinazolin-3-yl]ethoxy]phenyl]-5-methyl-thieno[3,2-b]pyridine-3-carboxylate (100 mg, 119.52 μmol, 7% yield, 85% purity) as an off white solid. LCMS: 711.43 [M+1].

### Step-4: Synthesis of 7-(5-chloro-2-(2-(2-methyl-6-(methyl(4-(trifluoromethyl)benzyl)amino)-4-oxo-5,6,7,8-tetrahydroquinazolin-3(4H)-yl)ethoxy)phenyl)-5-methylthieno[3,2-b]pyridine-3-carboxylic acid (Compound 201)

This intermediate was prepared by procedure F from methyl 7-[5-chloranyl-2-[2-[2-methyl-6-[methyl-[[4-[tris(fluorenyl)methyl]phenyl]methyl]amino]-4-oxidanylidene-5,6,7,8-tetrahydroquinazolin-3-yl]ethoxy]phenyl]-5-methyl-thieno[3,2-b]pyridine-3-carboxylate (80 mg, 112.49 μmol) afforded 7-[5-chloranyl-2-[2-[2-methyl-6-[methyl-[[4-[tris(fluorenyl)methyl]phenyl]methyl]amino]-4-oxidanylidene-5,6,7,8-tetrahydroquinazolin-3-yl]ethoxy]phenyl]-5-methyl-thieno[3,2-b]pyridine-3-carboxylic acid (16.6 mg, 23.45 μmol, 21% yield, 98.5% purity) as an off white solid. LCMS: 697.11 [M+1].

### 20. Experimental Procedure for Compound 538, Compound 537, Compound 550, Compound 570

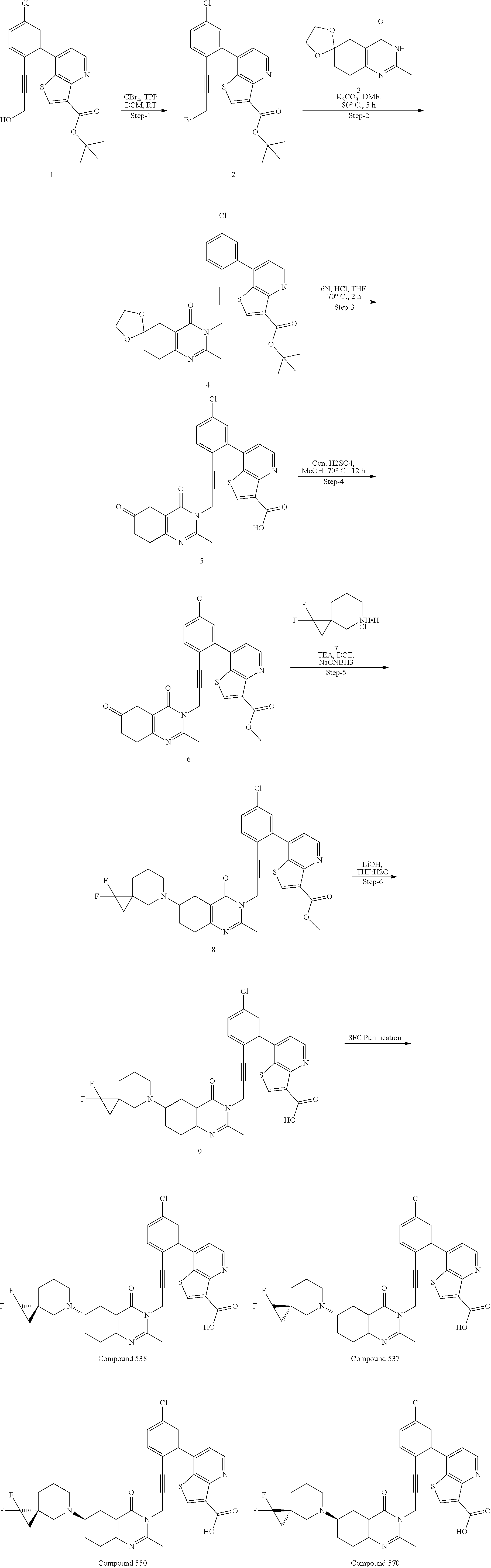

### Step-1: Synthesis of tert-butyl 7-(2-(3-bromoprop-1-yn-1-yl)-5-chlorophenyl)thieno[3,2-b]pyridine-3-carboxylate (2)

Triphenylphosphine (5.77 g, 22.01 mmol) was added to the stirred solution of 1,1-di(methyl)ethyl 7-[5-chloranyl-2-(3-oxidanylprop-1-ynyl)phenyl]thieno[3,2-b]pyridine-3-carboxylate (4.4 g, 11.00 mmol) in DCM (2.92 mL), at 0° C. then CBr₄ (7.30 g, 22.01 mmol, 2.13 mL) was added after 10 min at same temperature. Then reaction mixture was stirred at 25° C. for 2 h. Progress of reaction was monitored by LCMS. After completion of starting material, reaction was quenched with water (10 mL) and washed with DCM (50 mL×3). The combined organic layer was dried over anhydrous Na₂SO₄, filtered and concentrated to get the crude. Crude was subjected to flash chromatography by using mixture of methanol and ethyl acetate and hexane 15% to 20% as an eluent to afford desired product as a pale brown thick mass (1.5 g, 29%). LCMS; 464.13 [M+1].

### Step-2: Synthesis of tert-butyl 7-(5-chloro-2-(3-(2-methyl-4-oxo-7,8-dihydro-4H-spiro[quinazoline-6,2′-[1,3]dioxolan]-3(5H)-yl)prop-1-yn-1-yl)phenyl)thieno[3,2-b]pyridine-3-carboxylate (4)

Potassium carbonate (2.80 g, 20.25 mmol, 1.22 mL) was added to the solution of 2′-methylspiro[1,3-dioxolane-2,6′-3,5,7,8-tetrahydroquinazoline]-4′-one (1.5 g, 6.75 mmol) in DMF (10 mL) at 25° C. and the resulting reaction mixture stirred at 75° C. for 15 min. Then 1,1-di(methyl)ethyl 7-[2-(3-bromanylprop-1-ynyl)-5-chloranyl-phenyl]thieno[3,2-b]pyridine-3-carboxylate (2.50 g, 5.40 mmol) was added portion wise into the reaction mixture at 75° C. and the resulting reaction mixture was heated at 75° C. for 4 h. Progress of reaction was monitored by LCMS. The reaction mixture was cooled to 25° C. and quenched the reaction mixture by water. Then extract the reaction mixture with ethyl acetate (100 mL×3). The combined organic layer was washed with cold water (100 mL×1) and brine (100 mL×1), dried over Na₂SO₄ and filtered. The filtrate was concentrated under reduced pressure to afford crude as brown gummy liquid. This was purified by combi-flash column chromatography using 230-400 mesh silica gel and the desired product was eluted at 0-10% of MeOH in DCM as the eluent. Pure fractions (determined by TLC) were combined and evaporated under reduced pressure to afford the desired product as Light brown solid (1.4 g, 32%). LCMS; 604.11 [M+1].

### Step-3: Synthesis of 7-(5-chloro-2-(3-(2-methyl-4,6-dioxo-5,6,7,8-tetrahydroquinazolin-3(4H)-yl)prop-1-yn-1-yl)phenyl)thieno[3,2-b]pyridine-3-carboxylic acid (5)

4M HCl (5 mL) was added to the solution of 1,1-di(methyl)ethyl 7-[5-chloranyl-2-[3-(2′-methyl-4′-oxidanylidene-spiro[1,3-dioxolane-2,6′-7,8-dihydro-5H-quinazoline]-3′-yl)prop-1-ynyl]phenyl]thieno[3,2-b]pyridine-3-carboxylate (1.3 g, 2.15 mmol) in THF (5 mL) at room temperature. The resulting reaction mixture was stirred at 60° C. for 5 h. The progress of reaction was monitored by TLC and LCMS. After complete conversion of starting to desired product reaction mixture was concentrated under reduced pressure and PH was maintained neutral by using solid sodium bicarbonate at 0°. Aqueous layer washed with ethyl acetate (20 ml×3), the combined organic layer was washed with saturated brine solution (10 ml) and dried over sodium sulphate and concentrated under reduced pressure to obtain crude compound. Purification not performed (1 g, 92%). LCMS; [M+H]: 504 [M+1].

### Step-4: Synthesis of methyl 7-(5-chloro-2-(3-(2-methyl-4,6-dioxo-5,6,7,8-tetrahydroquinazolin-3(4H)-yl)prop-1-yn-1-yl)phenyl)thieno[3,2-b]pyridine-3-carboxylate (6)

H₂SO₄ (0.5 mL) was added to the solution of 7-[5-chloranyl-2-[3-[2-methyl-4,6-bis(oxidanylidene)-7,8-dihydro-5H-quinazolin-3-yl]prop-1-ynyl]phenyl]thieno[3,2-b]pyridine-3-carboxylic acid (1.0 g, 1.98 mmol) in Methanol (15 mL) at 0° C. The resulting reaction mixture was stirred at 70° C. for 12 h. The progress of reaction was monitored by LCMS. After complete conversion of starting to desired product reaction mixture was concentrated under reduced pressure. Residue was diluted with DCM (20 mL) and water (10 mL) separate the organic layer and aqueous layer washed with 10% MeOH:DCM (20 ml×3), the combined organic layer was washed with brine (10 ml) and dried over sodium sulphate and concentrated under reduced pressure to obtain crude compound. Used for the next reaction without purification (0.5 g, 48.5). LCMS; [M+H]: 518 [M+1].

### Step-5: Synthesis methyl 7-(5-chloro-2-(3-(6-(1,1-difluoro-5-azaspiro[2.5]octan-5-yl)-2-methyl-4-oxo-5,6,7,8-tetrahydroquinazolin-3(4H)-yl)prop-1-yn-1-yl)phenyl)thieno[3,2-b]pyridine-3-carboxylate (8)

Triethylamine (146.52 mg, 1.45 mmol, 201.81 μL) was added to the solution of 2,2-bis(fluorenyl)-5-azaspiro[2.5]octane; chlorane (177.25 mg, 965.28 μmol) in DCE (2 mL) at room temperature. The resulting reaction mixture was stirred at room temperature for 10 min. To this methyl 7-[5-chloranyl-2-[3-[2-methyl-4,6-bis(oxidanylidene)-7,8-dihydro-5H-quinazolin-3-yl]prop-1-ynyl]phenyl]thieno[3,2-b]pyridine-3-carboxylate (500 mg, 965.28 μmol) was added at room temperature. The resulting reaction mixture was stirred at 25° C. for 12 h. Then was added Sodium cyanoborohydride (151.65 mg, 2.41 mmol) at 0° C. The resulting reaction mixture was stirred at 25° C. for next 3 h. The progress of reaction was monitored by LCMS. After completion of starting material, reaction was quenched with water (2 mL) and washed with 10% MeOH:DCM (3×10 mL). The combined organic layer was dried over anhydrous Na₂SO₄, filtered and concentrated to get the crude compound (0.6 g, 20.5%). LCMS; 649 [M+1].

### Step-6: Synthesis of 7-(5-chloro-2-(3-(6-(1,1-difluoro-5-azaspiro[2.5]octan-5-yl)-2-methyl-4-oxo-5,6,7,8-tetrahydroquinazolin-3(4H)-yl)prop-1-yn-1-yl)phenyl)thieno[3,2-b]pyridine-3-carboxylic acid (9)

Lithium hydroxide (11.07 mg, 462.14 μmol) was added to the solution of methyl 7-[2-[3-[6-[2,2-bis(fluorenyl)-5-azaspiro[2.5]octan-5-yl]-2-methyl-4-oxidanylidene-5,6,7,8-tetrahydroquinazolin-3-yl]prop-1-ynyl]-5-chloranyl-phenyl]thieno[3,2-b]pyridine-3-carboxylate (100 mg, 154.05 μmol) in THF (0.5 mL) and Water (0.5 mL) at 0° C. The resulting reaction mixture was stirred at 25° C. for 12 h. The progress of reaction was monitored by TLC and LCMS. After completion of starting material, all volatiles were concentrated under reduced pressure to get crude residue. The residue was dissolved in water (1.0 mL) and pH adjusted to ˜3, by using saturated citric acid solution. resulting solid was collected by filtration and dried to afford crude. Crude was purified by Prep-HPLC (Column: X-SELECT-C18 (150*25), 10u ANL-PREPHW-2021-016 Mobile phase: 0.1% FA in H₂O:ACETONITRILE GRADIENT: (T % B): 0/10, 1/10, 11/40, 12/40, 12.1/98, 13/98, 13.1/10, 15/10 Flow Rate: 22 ml/min Diluent: ACN+H₂O) to afford desired product as an off white solid. Compound was submitted to SFC purification (SFC Method Conditions: Column: Chiralpak AD-H (250×4.6×5μ), % C02:60%, % Co solvent: 40% (30 mM Methanolic ammonia in Ethanol)) to afford methyl ester product as an off white solid four isomers ARG-1031, ARG-1032, ARG-1033, ARG-1034.1031: LCMS; [M+H]⁺: 635.24 (7.5 mg, 7.1%), 1032: LCMS; [M+H]⁺: 635.24 (7.6 mg, 7.2%), 1033: LCMS; [M+H]⁺: 635.24 (8.8 mg, 8.8%), 1034: LCMS; [M+H]⁺: 635.24 (2.1 mg, 2.1%).

The following analogues are synthesized using above protocol.

| | | | | LCMS |
|---|---|---|---|---|
| | | | | of 9 |
| Ex | Compound | Amine used | Method | [M + H]⁺ |
| 1 | Compound 535 Compound 534 | 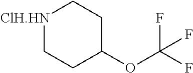 | A6 | 671.2 |
| 2 | Compound 365 Compound 410 | 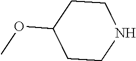 | A8 | 617.16 |

### 21. Experimental Procedure for Compound 443, Compound 442

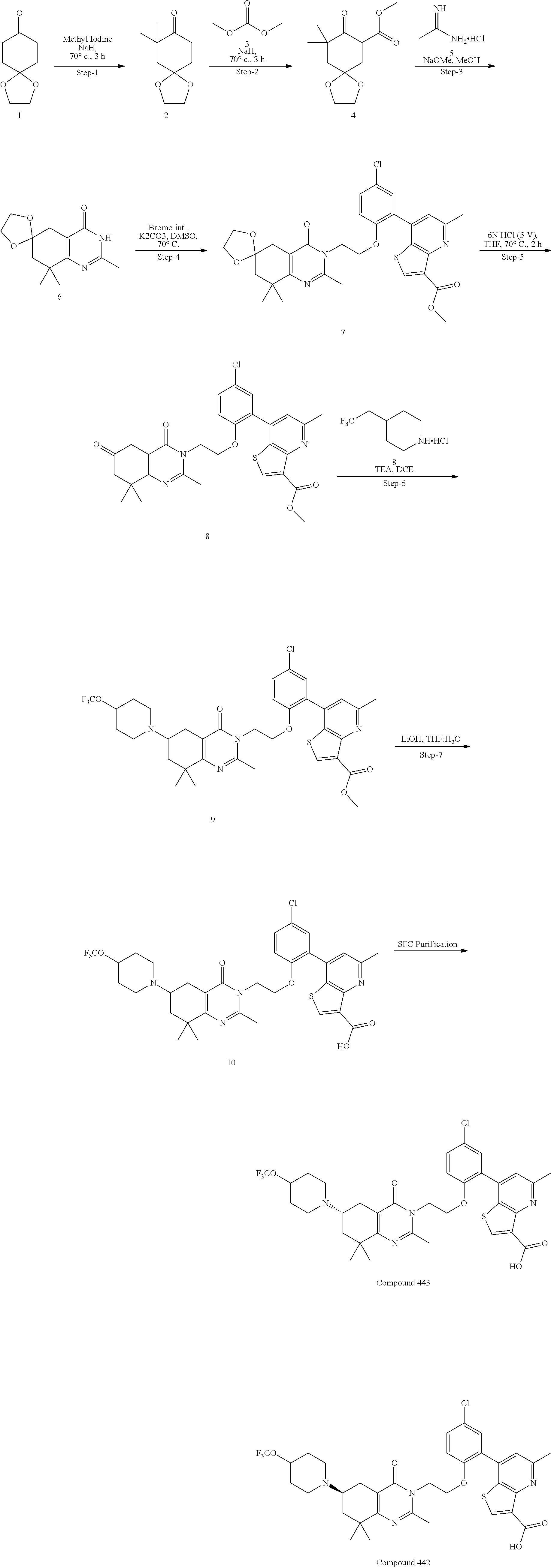

### Step-1: Synthesis of 7,7-dimethyl-1,4-dioxaspiro[4.5]decan-8-one (2)

Sodium hydride (in oil dispersion) 60% dispersion in mineral oil (5.40 g, 140.86 mmol, 60% purity) was added to stirred solution of 1,4-dioxaspiro[4.5]decan-8-one (10 g, 64.03 mmol) in THF (100 mL) at 0° C., Then stirred at 0° C. for 40 minutes. Then Iodomethane (19.99 g, 140.86 mmol, 8.77 mL) was added. Reaction mixture was stirred for 1 h at 0° C. and room temperature for 30 minutes. Progress of reaction was monitored by TLC. After completion of starting material, reaction was quenched with saturated ammonium chloride solution (50 mL) and washed with ethyl acetate (100 mL×3). The combined organic layer was dried over Na₂SO₄, filtered and concentrated to get the crude material. This was subjected to flash chromatography by using mixture of methanol and ethyl acetate and hexane 0% to 10% as an eluent to afford desired product as colourless liquid (6 g, 50.8%). 1H NMR: 1H-NMR (400 MHz, CDCl3) δ: 4.00 (s, 4H), 2.78-2.73 (t, J=6.00 Hz, 2H), 2.04-1.98 (t, J=6.80 Hz, 2H), 1.90 (s, 2H), 1.17 (s, 6H).

### Step-2: Synthesis of methyl 9,9-dimethyl-8-oxo-1,4-dioxaspiro[4.5]decane-7-carboxylate (4)

A suspension of Sodium hydride (in oil dispersion) 60% dispersion in mineral oil (499.15 mg, 21.71 mmol) in Dimethyl carbonate (9.07 g, 100.70 mmol, 8.48 mL) heated at 80° C. then solution of 7,7-di(methyl)-1,4-dioxaspiro[4.5]decan-8-one (2 g, 10.86 mmol) in Dimethyl carbonate (9.07 g, 100.70 mmol, 8.48 mL), was added drop wise at same temp and stirred for another 2 h. Reaction was monitor by TLC. Reaction mass quenched with ice cold water solution (50 mL) and washed by ethyl acetate (3×100 mL), combined organic layer and dried over sodium sulphate and concentrated under reduced pressure and get crude of methyl 7,7-di(methyl)-8-oxidanylidene-1,4-dioxaspiro[4.5]decane-9-carboxylate (2 g, 69.00%) as a Brown liquid. Crude was subjected to flash chromatography by using a mixture of ethyl acetate and petroleum ether 7-8% as an eluent to afford the desired product as a white solid (2 g, 69%). LCMS; 242.43 [M+1].

### Step-3: Synthesis of 2,8,8-trimethyl-3,5,7,8-tetrahydro-4H-spiro[quinazoline-6,2′-[1,3]dioxolan]-4-one (6)

Sodium methoxide, ca 30% w/w in methanol (8.92 g, 165.11 mmol, 9.20 mL) and acetamidine; chlorane (1.17 g, 12.38 mmol), 97% (358.1 g, 3.79 mol) was added to a stirred solution of methyl 7,7-di(methyl)-8-oxidanylidene-1,4-dioxaspiro[4.5]decane-9-carboxylate (2 g, 8.26 mmol) in Methanol (468.07 mL) at room temperature. Then the reaction mixture was slowly heated to 80° C. and stirred for 12 h. The reaction was monitored by TLC. Methanol was removed from the reaction mixture by concentrating under reduced pressure and getting crude. The crude was diluted in quenched water and neutralized with saturated citric acid solution to pH (˜7) to get white solid, filtered the white solid and washed with diethyl ether and dried under reduced pressure to get required compound as an off white solid (1.4 g, 67.6%). LCMS; 251.23 [M+1].

### Step-4: Synthesis of methyl 7-(5-chloro-2-(2-(2,8,8-trimethyl-4-oxo-7,8-dihydro-4H-spiro[quinazoline-6,2′-[1,3]dioxolan]-3(5H)-yl)ethoxy)phenyl)-5-methylthieno[3,2-b]pyridine-3-carboxylate (7)

Potassium carbonate, anhydrous, 99% (2.32 g, 16.78 mmol, 1.01 mL) was added to the solution of 2′,8′,8′-tri(methyl)spiro[1,3-dioxolane-2,6′-5,7-dihydro-3H-quinazoline]-4′-one (1.4 g, 5.59 mmol) in DMSO (7 mL) at 25° C. and the resulting reaction mixture stirred at 75° C. for 15 min. Then methyl 7-(2-(2-bromoethoxy)-5-chlorophenyl)-5-methylthieno[3,2-b]pyridine-3-carboxylate (2.22 g, 5.03 mmol) was added slowly portion wise into the reaction mixture at 75° C. and the resulting reaction mixture was heated at 75° C. for 4 h. Progress of reaction was monitored by LCMS. The reaction mixture was cooled to 25° C. and quenched the reaction mixture by aqueous solution. Then extract the reaction mixture with ethyl acetate (100 mL×3). The combined organic layer was washed with cold water (100 mL×1) and brine (100 mL×1), dried over Na₂SO₄ and filtered. The filtrate was concentrated under reduced pressure to afford crude as brown gummy liquid. This was purified by combi-flash column chromatography using 230-400 mesh silica gel and the desired product was eluted at 0-10% of MeOH in DCM as the eluent. Pure fractions (determined by TLC) were combined and evaporated under reduced pressure to afford the desired product as white solid (1.4 g, 37.7%). LCMS; 610 [M+1].

### Step-5: Synthesis methyl 7-(5-chloro-2-(2-(2,8,8-trimethyl-4,6-dioxo-5,6,7,8-tetrahydroquinazolin-3(4H)-yl)ethoxy)phenyl)-5-methylthieno[3,2-b]pyridine-3-carboxylate (8)

4N HCl (8.5 mL) was added to the solution of methyl 7-(5-chloro-2-(2-(2-methyl-4-oxo-7,8-dihydro-4H-spiro[quinazoline-6,2′-[1,3]dioxolan]-3(5H)-yl)ethoxy)phenyl)-5-methylthieno[3,2-b]pyridine-3-carboxylate (1.4 g, 2.29 mmol) in THF (8.5 mL) at RT. The resulting reaction mixture was stirred at 60° C. for 5 h. The progress of reaction was monitored by TLC and LCMS. Volatiles were evaporated under reduced pressure and residue was basified with saturated solution of NaHCO₃ and washed with ethyl acetate (20 mL×3), the combined organic layer was washed with brine (10 ml) and dried over sodium sulphate and concentrated under reduced pressure to obtain crude compound (1.0 g, 61.5%).

LCMS; 566 [M+1].

### Step-6: Synthesis of methyl 7-(5-chloro-2-(2-(2,8,8-trimethyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylate (9)

Triethylamine (53.63 mg, 529.98 μmol, 73.87 μL) was added to the solution of chlorane; 4-[tris(fluorenyl)methoxy]piperidine (145.29 mg, 706.63 μmol) in DCE (2 mL) at room temperature. The resulting reaction mixture was stirred at room temperature for 10 min. Then was added methyl 7-[5-chloranyl-2-[2-[2,8,8-tri(methyl)-4,6-bis(oxidanylidene)-5,7-dihydroquinazolin-3-yl]ethoxy]phenyl]-5-methyl-thieno[3,2-b]pyridine-3-carboxylate (200 mg, 353.32 μmol) and titanium tetraisopropoxide (753.13 mg, 2.65 mmol, 788.62 μL) at room temperature. The resulting reaction mixture was stirred at 25° C. for 16 h. To this Sodium cyanoborohydride (55.51 mg, 883.29 μmol) was added at 0° C. The resulting reaction mixture was stirred at 25° C. for next 24 h. The progress of reaction was monitored by LCMS. After completion of starting material, reaction was quenched with water (2 mL) and washed with 10% MeOH:DCM (3×10 mL). The combined organic layer was dried over Na₂SO₄, filtered and concentrated to get the crude. This was subjected to flash chromatography by using mixture of methanol and DCM 5%-10% as an eluent to afford desired product as an off white solid. Since transesterification observed in reaction, the product isolated was isopropyl ester (0.18 g, 20.1%). LCMS; 719.21 And 747.3 (isopropyl ester) [M+1].

### Step-7: Synthesis of 7-(5-chloro-2-(2-(2,8,8-trimethyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylic acid

Lithium hydroxide (59.94 mg, 2.50 mmol) was added to the solution of methyl 7-(5-chloro-2-(2-(2,8,8-trimethyl-4-oxo-6-(4-(trifluoromethoxy)piperidin-1-yl)-5,6,7,8-tetrahydroquinazolin-3(4H)-yl)ethoxy)phenyl)-5-methylthieno[3,2-b]pyridine-3-carboxylate (180 mg, 250.27 μmol) in THF (0.5 mL) and Water (0.5 mL) was added at 0° C. The resulting reaction mixture was stirred at 25° C. for 4 h. The progress of reaction was monitored by TLC and LCMS. After completion of starting material, all volatiles were concentrated under reduced pressure to get crude residue. The residue was dissolved in water (1.0 mL) and pH adjusted to ˜3, by using saturated citric acid solution. resulting solid was collected by filtration and dried to afford crude. Crude was purified by Prep-HPLC (Column Name X-BRIDGE-C18 (150*19 mm), 5u Column No #MCL-PREP-COL-2022-068 Mobile Phase-A 10 mM Ammonium Bicarbonate in water Mobile Phase-B Acetonitrile Gradient program (T/% B) 0/15, 12/50, 12.1/98, 14/98, 14.1/15, 16/15 Flow Rate (mL/minute) 16 Sample Loading(mg/Injection) 15) to afford desired product as a white solid. Compound was submitted to SFC purification (SFC Method Conditions: Column: Chiralpak IA (250×4.6×5μ), Co-solvent: 0.5% Methanolic ammonia in Methanol:DCM(70:30)) to afford desired product as an off white solid four isomers ARG-754 (24.4 mg, 13.8), ARG-755 (16.4 mg, 9.2%). LCMS; 705.19 [M+1].

The following analogues are prepared using above protocol.

| | | | | LCMS of 10 |
|---|---|---|---|---|
| Ex | Compound | Amine | Method | [M + H]⁺ |
| 2 | Compound 441 Compound 440 Compound 439 Compound 438 | 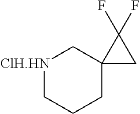 | A3 | 725.3 |
| 3 | Compound 492 Compound 500 | 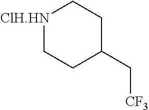 | A3 | 745.3 |

### 22. Experimental Protocol of Compound 102 and Compound 103

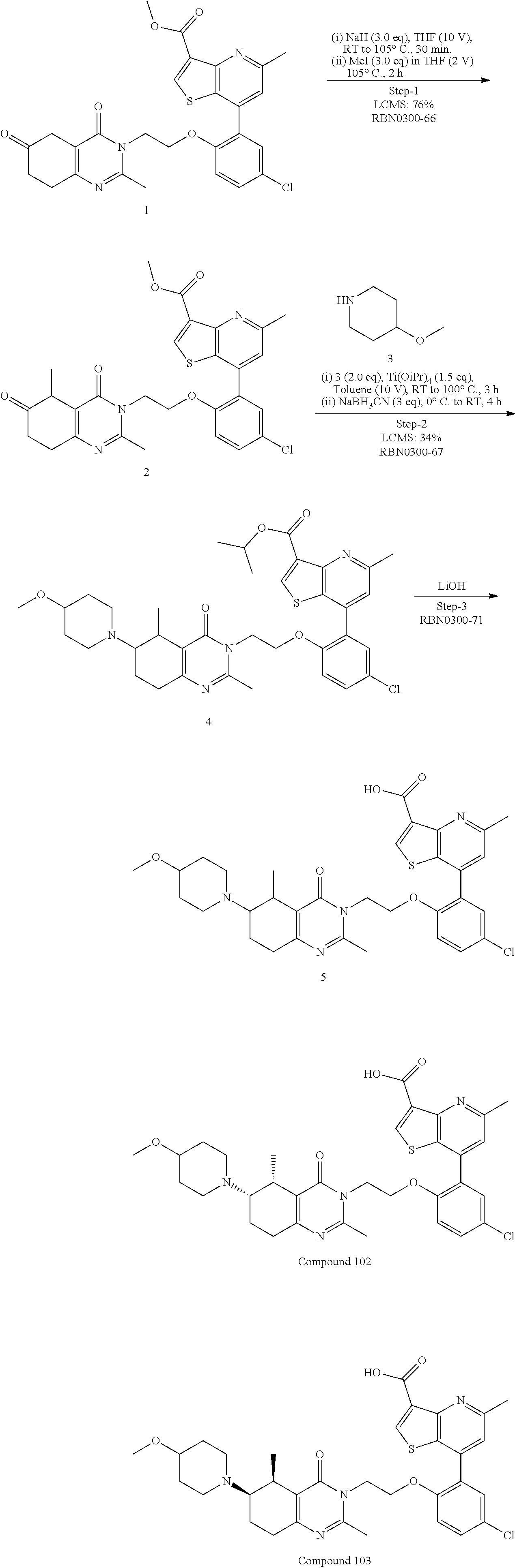

### Step-1: Synthesis of methyl 7-(5-chloro-2-(2-(2,5-dimethyl-4,6-dioxo-5,6,7,8-tetrahydroquinazolin-3(4H)-yl)ethoxy)phenyl)-5-methylthieno[3,2-b]pyridine-3-carboxylate (2)

Sodium hydride (in oil dispersion) 60% dispersion in mineral oil (0.240 g, 9.29 mmol) was added to the solution of methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (1 g, 1.86 mmol) in THF (20 mL) at room temperature and slowly heat to 105° C. and stirred for 30 min. After solution of methyl iodide (0.792 g, 5.58 mmol, 347.13 L) in THF (5 mL) was added slowly dropwise at 105° C. The resulting reaction mixture was stirred at the same temperature for 2 hr. The reaction mixture was cooled to 0° C. and quenched with saturated ammonium chloride solution and extract with ethyl acetate (100 mL×3). The combined organic layer was washed with cold water (100 mL) and brine solution (100 mL×1), the organic layer was dried over Na₂SO₄ and filtered. The filtrate was concentrated under reduced pressure to afford crude as brown gummy solid. The crude was purified by combi-flash column chromatography using 230-400 mesh silica gel and the desired product was eluted at 5-10% of MeOH in DCM as the eluent. Pure fractions (determined by TLC) were combined and evaporated under reduced pressure to afford methyl 7-(5-chloro-2-(2-(2,5-dimethyl-4,6-dioxo-5,6,7,8-tetrahydro quinazolin-3(4H)-yl)ethoxy)phenyl)-5-methylthieno[3,2-b]

pyridine-3-carboxylate (400 mg, 40%) as a brown solid. LCMS; 552.35 [M+1].

### Step-2: Synthesis of isopropyl 7-(5-chloro-2-(2-(6-(4-methoxypiperidin-1-yl)-2,5-dimethyl-4-oxo-5,6,7,8-tetrahydroquinazolin-3(4H)-yl)ethoxy)phenyl)-5-methylthieno[3,2-b]pyridine-3-carboxylate (4)

Titanium Isopropoxide (205.94 mg, 724.59 μmol, 215.64 μL) was added to the solution of methyl 7-[5-chloranyl-2-[2-[2,5-di(methyl)-4,6-bis(oxidanylidene)-7,8-dihydro-5H-quinazolin-3-yl]ethoxy]phenyl]-5-methyl-thieno[3,2-b]pyridine-3-carboxylate (200 mg, 362.29 mol) and 4-methoxypiperidine (41.73 mg, 362.29 μmol) in Toluene (2 mL) at room temperature and slowly heated to 100° C. and stirred for 3 h. After the resulting reaction mixture was cooled to 0° C. and sodium cyanoborohydride (68.30 mg, 1.09 mmol) was added slowly portion wise and allowed room temperature and stirred for another 4 h. The reaction mixture was poured into ice-cold water (50 mL) and filtered through a celite bed followed by washes with DCM (50 mL×3). The organic layer was separated and the aqueous layer was washed again with DCM (50 mL). The combined organic layer was concentrated under reduced pressure to afford isopropyl 7-(5-chloro-2-(2-(6-(4-methoxypiperidin-1-yl)-2,5-dimethyl-4-oxo-5,6,7,8-tetrahydroquinazolin-3(4H)-yl)ethoxy)phenyl)-5-methylthieno[3,2-b]pyridine-3-carboxylate (250 mg, 50%) as a brown solid. LCMS; 679.37 [M+1].

### Step-3: Synthesis of 7-(5-chloro-2-(2-(6-(4-methoxypiperidin-1-yl)-2,5-dimethyl-4-oxo-5,6,7,8-tetrahydroquinazolin-3(4H)-yl)ethoxy)phenyl)-5-methylthieno[3,2-b]pyridine-3-carboxylic acid (5)

Lithium hydroxide monohydrate (25 mg, 1.02 mmol) was added to the solution of 1-methylethyl 7-[5-chloranyl-2-[2-[6-(4-methoxy-1-piperidyl)-2,5-di(methyl)-4-oxidanylidene-5,6,7,8 etrahydroquinazolin-3-yl]ethoxy]phenyl]-5-methyl-thieno[3,2-b]pyridine-3-carboxylate (230 mg, 338.60 μmol) in a mixture of solvents THF:H₂O (2 mL:0.5 mL) at room temperature and stirred for 2 hr. The reaction mass was acidified with aq. 1 N HCl solution (pH 4-6). Volatiles were removed under reduced pressure, washed 10-15% MeOH in Dichloromethane (50 mL×2). The combined organic layer was concentrated under reduced pressure to obtain the crude material. Crude was purified by Prep-HPLC (Column Name X-BRIDGE-C18 (150*19 mm), 5u Column No #MCL-PREP-COL-2022-068 Mobile Phase-A 10 mM Ammonium Bicarbonate in water Mobile Phase-B Acetonitrile Gradient program (T/% B) 0/15, 12/50, 12.1/98, 14/98, 14.1/15, 16/15 Flow Rate (mL/minute) 16 Sample Loading(mg/Injection) 15) to afford 55 mg desired product as a off white solid. Isomer was separated to SFC Method Conditions: Column: CHIRALPAK IA (4.6*250 mm) 5 μm Co-solvent: 0.5% (7N Methanolic Ammonia) in MeOH:IPA (1:1) Total flow: 4 mL/min % of CO2 60% of Co-Solvent: 40 ABPR: 1500 psi Temperature: 30° C. to afford desired product as an off white solid two isomers. LCMS; 637.3 [M+1].

### 23. Experimental Procedure for Compound 123

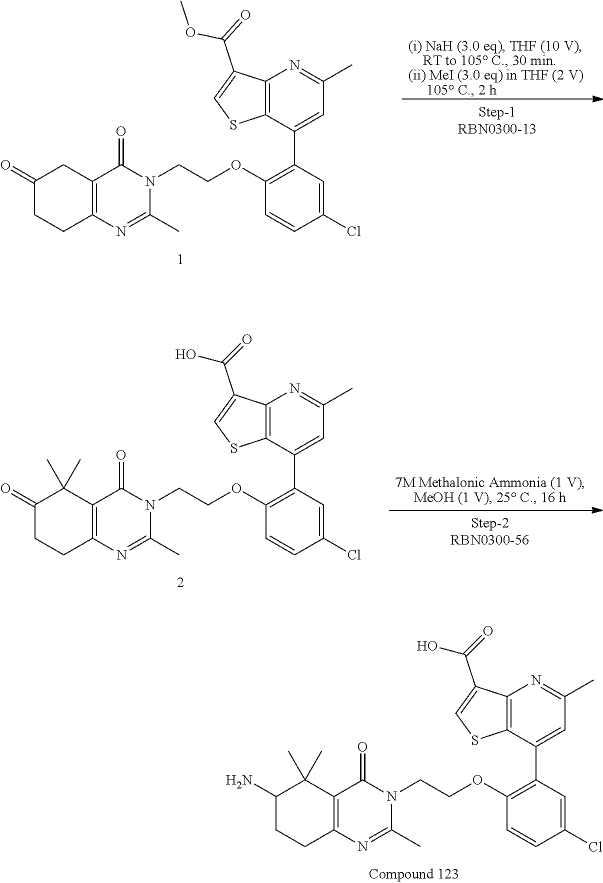

### Step-1: Synthesis of 7-(5-chloro-2-(2-(2,5,5-trimethyl-4,6-dioxo-5,6,7,8-tetrahydroquinazolin-3(4H)-yl)ethoxy)phenyl)-5-methylthieno[3,2-b]pyridine-3-carboxylic acid (2)

Sodium hydride (in oil dispersion) 60% dispersion in mineral oil (25.64 mg, 1.12 mmol) was added to the solution of methyl 7-[5-chloranyl-2-[2-[2-methyl-4,6-bis(oxidanylidene)-7,8-dihydro-5H-quinazolin-3-yl]ethoxy]phenyl]-5-methyl-thieno[3,2-b]pyridine-3-carboxylate (200 mg, 371.74 μmol) in THF (4.95 mL) at room temperature and slowly heated to 105° C. and stirred for 30 min. After solution of methyl iodide (158.29 mg, 1.12 mmol, 69.43 μL) in THF (0.5 mL) was added slowly dropwise at 105° C. The resulting reaction mixture was stirred at the same temperature for 2 hr. The reaction mixture was cooled to 0° C. and quenched with saturated ammonium chloride solution and extract with EtOAc (100 mL×3). The combined organic layer was washed with cold water (100 mL) and brine solution (100 mL), the organic layer was dried over Na₂SO₄ and filtered. The filtrate was concentrated under reduced pressure to afford crude as brown gummy solid. The crude was purified by combi-flash column chromatography using 230-400 mesh silica gel and the desired product was eluted at 5-10% of MeOH in DCM the eluent. Pure fractions (determined by TLC) were combined and evaporated under reduced pressure to afford 7-(5-chloro-2-(2-(2,5,5-trimethyl-4,6-dioxo-5,6,7,8-tetrahydroquinazolin-3(4H)-yl)ethoxy)phenyl)-5-methyl thieno[3,2-b]pyridine-3-carboxylic acid (80 mg, 39%) as brown solid. ¹H NMR (DMSO-d6, 400 MHz): 13.68 (brs, 1H), 8.83 (s, 1H), 7.58 (d, J=8.00 Hz, 2H), 7.45 (s, 1H), 7.33 (d, J=8.00 Hz, 1H), 4.45-4.32 (m, 2H), 4.25-4.08 (m, 1H), 3.17 (s, 1H), 2.79-2.67 (m, 7H), 1.63 (s, 3H), 1.35 (s, 6H).

### Step-2: Synthesis of 7-(2-(2-(6-amino-2,5,5-trimethyl-4-oxo-5,6,7,8-tetrahydroquinazolin-3(4H)-yl)ethoxy)-5-chlorophenyl)-5-methylthieno[3,2-b]pyridine-3-carboxylic acid (Compound 123)

7 M ammonia in methanol (3 mL) was added to the solution of 7-[5-chloranyl-2-[2-[2,5,5-tri(methyl)-4,6-bis(oxidanylidene)-7,8-dihydroquinazolin-3-yl]ethoxy]phenyl]-5-methyl-thieno[3,2-b]pyridine-3-carboxylic acid (80 mg, 144.92 μmol) in methanol (1 mL) at room temperature, the resulting reaction mixture was stirred at same temperature for 16 hr. After the resulting reaction mixture was cooled to 0° C. and sodium cyanoborohydride (27.32 mg, 434.75 mol) was added slowly portion wise and allowed room temperature and stirred for another 4 hr. Concentrate the reaction mass under reduced pressure and diluted with water (30 mL). Then neutralized by using saturated citric acid solution and extract with 10% MeOH in DCM (50 mL×3), the organic layer was dried over Na₂SO₄ and filtered. The filtrate was concentrated under reduced pressure to afford crude as brown gummy solid. The crude was purified by Prep. HPLC to afford 7-(2-(2-(6-amino-2,5,5-trimethyl-4-oxo-5,6,7,8-tetrahydroquinazolin-3(4H)-yl)ethoxy)-5-chlorophenyl)-5-methylthieno[3,2-b]pyridine-3-carboxylic acid as an off white solid. LCMS; 553.25 [M+1].

### 24. Experimental Procedure for Compound 629, Compound 626, Compound 628, Compound 634

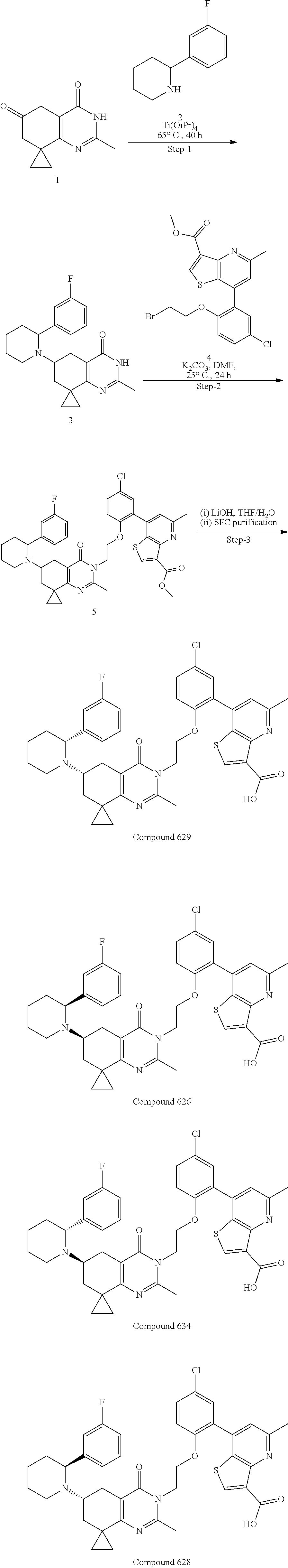

### Step-1: synthesis of 6′-(2-(3-fluorophenyl)piperidin-1 yl)-2′-methyl-3′,5′,6′,7′-tetrahydro-4′H-spiro[cyclopropane-1,8′-quinazolin]-4′-one (3)

Titanium(IV) isopropoxide (6.96 g, 24.48 mmol) was added to a mixture of 2-methyl spiro[5,7-dihydro-3H-quinazoline-8,1′-cyclopropane]-4,6-dione 1 (500 mg, 2.45 mmol) and 2-(3-fluorenylphenyl)piperidine 2 (570.46 mg, 3.18 mmol) at 25° C. The resulting reaction mixture was stirred at 65° C. for 40 h. Then added Sodium cyanoborohydride (923.10 mg, 14.69 mmol) in three portions at 25° C. The resulting reaction mixture was stirred at 25° C. for 2 h. The reaction progress monitored by LCMS. It was poured in ice cold water (30 mL). It was diluted with 10% MeOH in DCM (50 mL). The reaction mass filtered through celite. Celite bed washed with 10% MeOH in DCM (50 mL). The organic layer was separated. The separated organic layer dried over Na₂SO₄ and concentrated to get crude compound (1.1 g). This was purified over Biotage (prepacked 40 g Si-gel column). The column eluted with 5% MeOH in DCM. The collected pure fraction was concentrated under reduced pressure to get 3 (425 mg) as a brown solid. LCMS: 368.2 [M+1].

### Step-2: synthesis of methyl 7-(5-chloro-2-(2-(6′-(2-(3-fluorophenyl)piperidin-1-yl)-2′-methyl-4′-oxo-6′,7′-dihydro-4′H-spiro[cyclopropane-1,8′-quinazolin]-3′(5′H)yl)ethoxy)phenyl)-5-methylthieno[3,2-b]pyridine-3-carboxylate (5)

Potassium carbonate (157.97 mg, 1.14 mmol) was added to a mixture of 6-[2-(3-fluorophenyl)-1-piperidyl]-2-methyl-spiro[3,5,6,7-tetrahydroquinazoline-8,1′-cyclopropane]-4-one 3 (420 mg, 1.14 mmol) and methyl 7-[2-(2-bromoethoxy)-5-chloro-phenyl]-5-methyl-thieno[3,2-b]pyridine-3-carboxylate 4 (503.76 mg, 1.14 mmol) in DMF (4 mL) at 25° C. It was stirred for 24 h at the same temperature. The progress of the reaction monitored by LCMS. The reaction mass poured in ice-cold water (25 mL) with stirring. The obtained solid was filtered and dried under suction to get crude compound 5 (600 mg) as a brown solid. This was used for the next step without purification. LCMS: 727.2 [M+1].

### Step-3: Synthesis of 7-(5-chloro-2-(2-(6′-(2-(3-fluorophenyl)piperidin-1-yl)-2′-methyl-4′-oxo-6′,7′-dihydro-4′H-spiro[cyclopropane-1,8′-quinazolin]-3′(5′H)-yl)ethoxy)phenyl)-5-methylthieno[3,2-b]pyridine-3-carboxylic acid (Compound 629, Compound 626, Compound 628, Compound 634)

Lithium hydroxide monohydrate (103.86 mg, 2.47 mmol) was added in portions to an ice cold solution of methyl 7-[5-chloro-2-[2-[6-[2-(3-fluorophenyl)-1-piperidyl]-2-methyl-4-oxo-spiro[6,7-dihydro-5H-quinazoline-8,1′-cyclopropane]-3-yl]ethoxy]phenyl]-5-methyl-thieno[3,2-b]pyridine-3-carboxylate (600 mg, 824.99 μmol) in THF (5.0 mL) and water (2.0 mL) at 0° C. It was stirred at 25° C. for 4 h. The completion of reaction observed by TLC. Reaction mixture was acidified with 6M HCl till pH 6 and concentrated under reduced pressure. Residue was partitioned between water (5 mL) and 10% MeOH in DCM (20 mL). The separated organic layer was dried over Na₂SO₄ and concentrated to get crude (600 mg). The crude (600 mg) was purified by prep-HPLC, collected two peaks, Peak-A (103 mg) and Peak-B (74 mg). Further Peak-A (103 mg) purified by SFC and lyophilized to get 1173 (8.3 mg) and 1174 (9.4 mg). Further Peak-B (74 mg) purified by SFC and lyophilized to get 1175 (10.4 mg) and 1176 (14 mg).

629: LCMS: 713.34 [M+1], ¹H-NMR (400 MHz, DMSO-d6): δ 8.57 (s, 1H), 7.54 (dd, J=2.80, 9.00 Hz, 1H), 7.42 (d, J=2.40 Hz, 1H), 7.33-7.28 (m, 3H), 7.13 (dd, J=10.40, 10.80 Hz, 2H), 7.01-6.96 (m, 1H), 4.33-4.24 (m, 2H), 4.06-3.96 (m, 2H), 3.55-3.53 (m, 1H), 2.95 (d, J=11.20 Hz, 1H), 2.79-2.65 (m, 1H), 2.66 (s, 3H), 2.46-2.22 (m, 4H), 1.90-1.64 (m, 3H), 1.58 (s, 3H), 1.50-1.35 (m, 3H), 1.29-1.20 (m, 2H), 1.04-0.64 (m, 3H).

626: LCMS: 713.34 [M+1], ¹H-NMR (400 MHz, DMSO-d6): δ 8.58 (s, 1H), 7.54 (dd, J=2.40, 9.00 Hz, 1H), 7.42 (d, J=2.80 Hz, 1H), 7.33-7.28 (m, 3H), 7.13 (dd, J=9.60, 18.0 Hz, 2H), 7.01-6.96 (m, 1H), 4.34-4.23 (m, 2H), 4.08-3.95 (m, 2H), 3.54 (d, J=8.00 Hz, 1H), 2.95 (d, J=10.80 Hz, 1H), 2.82-2.76 (m, 1H), 2.66 (s, 3H), 1.90-1.69 (m, 4H), 1.75-1.69 (m, 3H), 1.64 (s, 3H), 1.47-1.24 (m, 5H), 1.06-0.39 (m, 3H).

634: LCMS: 713.34 [M+1], ¹H-NMR (400 MHz, DMSO-d6): δ 1H-NMR (400 MHz, DMSO-d6): δ 8.55 (s, 1H), 7.53 (dd, J=2.40, 8.80 Hz, 1H), 7.41 (d, J=2.80 Hz, 1H), 7.34-7.28 (m, 3H), 7.18-7.12 (m, 2H), 7.01-6.97 (m, 1H), 4.32-4.20 (m, 2H), 4.06-3.95 (m, 2H), 3.48-3.32 (m, 1H), 3.09-3.06 (m, 1H), 2.67-2.51 (m, 3H), 2.52-2.18 (m, 3H), 1.86 (t, J=12.40 Hz, 1H), 1.74-1.42 (m, 6H), 1.33-1.17 (m, 6H), 1.07-0.32 (m, 3H).

628: LCMS: 713.34 [M+1], ¹H-NMR (400 MHz, DMSO-d6): δ 8.68 (s, 1H), 7.55 (dd, J=2.80, 9.00 Hz, 1H), 7.42 (d, J=2.80 Hz, 1H), 7.34-7.29 (m, 3H), 7.19-7.13 (m, 2H), 7.01-6.96 (m, 1H), 4.34-4.22 (m, 2H), 4.06-3.95 (m, 2H), 3.48-3.46 (m, 1H), 3.10-3.07 (m, 1H), 2.69-2.67 (m, 1H), 2.66 (s, 3H), 2.43-2.15 (m, 3H), 1.86 (t, J=12.40 Hz, 1H), 1.74-1.56 (m, 3H), 1.65 (s, 3H), 1.52-1.49 (m, 2H), 1.33-1.23 (3H), 0.63-0.39 (m, 3H). 34. Experimental procedure for Compound 625, Compound 633, Compound 624, Compound 630:

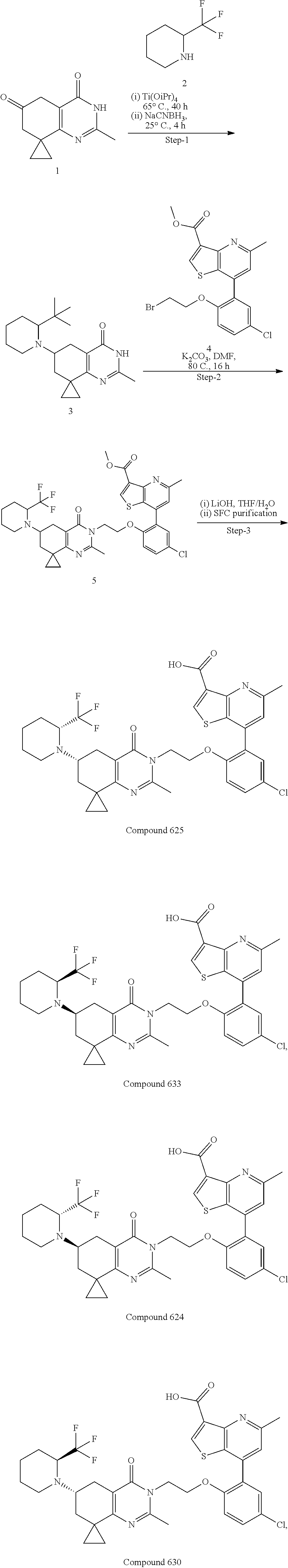

625, 633, 624 and 630 was synthesized by using the experimental procedure 33.

Purified by prep-HPLC, collected two peaks, Peak-A (102 mg) and Peak-B (92 mg). Further Peak-A (102 mg) purified by SFC and lyophilized to get 1177 (17.2 mg) and 1178 (9.3 mg). Further Peak-B (92 mg) purified by SFC and lyophilized to get 1179 (19.9 mg) and 1180 (17.7 mg).

625: LCMS: 687.30 [M+1], ¹H-NMR (400 MHz, DMSO-d6): δ 8.75 (s, 1H), 7.57 (dd, J=2.80, 8.80 Hz, 1H), 7.44 (d, J=2.80 Hz, 1H), 7.38-7.28 (m, 2H), 4.35-4.29 (m, 2H), 4.09-4.03 (m, 2H), 3.55-3.50 (m, 1H), 2.91-2.68 (m, 6H), 2.37-2.02 (m, 2H), 1.73-1.71 (m, 2H), 1.63 (s, 3H), 1.56-1.23 (m, 7H), 0.76-0.67 (m, 3H).

633: LCMS: 687.30 [M+1], ¹H-NMR (400 MHz, DMSO-d6): 8.72 (s, 1H), 7.57 (dd, J=2.80, 8.80 Hz, 1H), 7.44 (d, J=2.80 Hz, 1H), 7.37-7.32 (m, 2H), 4.35-4.29 (m, 2H), 4.09-4.03 (m, 2H), 3.57-3.55 (m, 1H), 3.04-2.86 (m, 2H), 2.76-2.64 (m, 5H), 2.37-2.30 (m, 1H), 2.07 (t, J=12.4 Hz, 1H), 171.71 (m, 2H), 1.63 (s, 3H), 1.56-1.23 (m, 6H), 0.76-0.70 (m, 3H).

624: LCMS: 687.26 [M+1], ¹H-NMR (400 MHz, DMSO-d6): δ 8.76 (s, 1H), 7.57 (dd, J=2.40, 9.00 Hz, 1H), 7.44 (d, J=2.80 Hz, 1H), 7.41-7.26 (m, 2H), 4.35-4.29 (m, 2H), 4.08-4.03 (m, 2H), 3.56-3.51 (m, 1H), 3.04-3.01 (m, 1H), 2.79-2.60 (m, 6H), 2.37-2.04 (m, 2H), 1.73-1.35 (m, 9H), 1.33-1.23 (m, 2H), 0.75-0.63 (m, 3H).

630: LCMS: 687.30 [M+1], ¹H-NMR (400 MHz, DMSO-d6): δ 8.78 (s, 1H), 7.57 (dd, J=2.80, 9.00 Hz, 1H), 7.44 (d, J=2.80 Hz, 1H), 7.38-7.32 (m, 2H), 4.36-4.29 (m, 2H), 4.08-4.03 (m, 2H), 3.57-3.52 (m, 1H), 3.06-3.02 (m, 1H), 2.79-2.51 (m, 6H), 2.36-2.04 (m, 2H), 1.73-1.30 (m, 11H), 0.74-0.61 (m, 3H).

### 35. Experimental Procedure for Compound 380, Compound 379

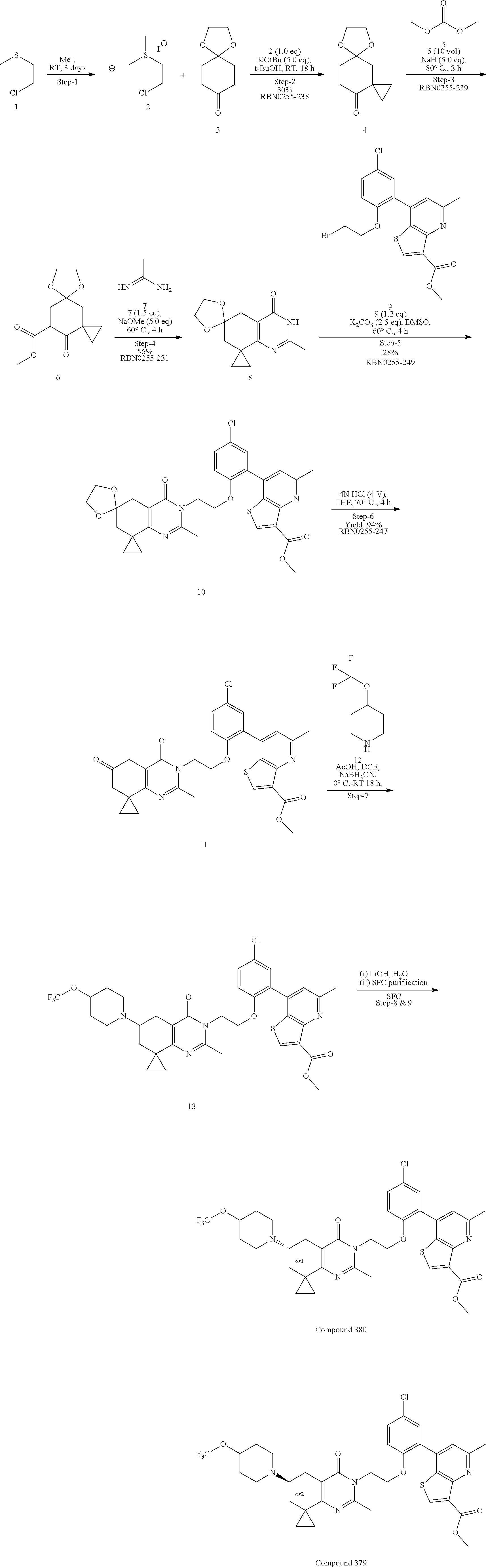

### Step 1: Synthesis of 2-chloroethyl-di(methyl) sulfonium iodide

To a stirred solution of 1-chloranyl-2-methylsulfanyl-ethane (25 g, 226.03 mmol) was added Mel (160.41 g, 1130 mmol) dropwise at 0° C. Reaction mixture was stirred at room temperature for 36 h. All volatiles were concentrated under reduced pressure to afford to crude compound. The crude compound was stirred in a diethyl ether solid formed was filtered through Buckner funnel and residue was washed with di ethyl ether and dried under reduced pressure to afford a required compound 2-chloroethyl-di(methyl) sulfonium iodide as Brown solid (45 g, 78%).

Note: Skin exposure with methyl iodide furnishes blisters.

¹H NMR (DMSO-d6, 400 MHz): δ 4.16 (t, J=8.0 Hz, 2H), 3.01 (s, 6H).

### Step 2: Synthesis of 6,9-dioxadispiro[2.1.45.33]dodecan-12-one

To a stirred of solution of Potassium tertiary butoxide (9.34 g, 83.24 mmol) in t-BuOH (50 mL) was added 1,4-dioxaspiro[4.5]decan-8-one (10 g, 64.03 mmol). Reaction mixture stirred at room temperature for 15 min. to this (chloromethyl)dimethylsulfonium iodide (12.22 g, 51.22 mmol) was added portion wise at room temperature. Reaction mixture stirred at room temperature for 15 h. Reaction was monitored by the TLC. After completion of reaction, reaction mixture was quenched with water and washed with ethyl acetate (2×200 mL) both organic layers were combined and washed with water, brine solution and dried over Na2SO4 and concentrated under reduced pressure and get crude compound. Crude compound was purified by combi flash column chromatography, eluted with 15-20% ethyl acetate in pet ether as pale-yellow oily liquid (3.5 g, 30%).

¹H NMR (DMSO-d6, 400 MHz): δ 0.68 (t, J=4.0 Hz, 2H), 1.26 (t, J=4.0 Hz, 2H), 1.99 (s, 2H), 2.11 (t, J=8.0 Hz, 2H), 2.574 (t, J=8.0 Hz, 2H), 4.01 (m, 6H).

### Step 3: Synthesis of methyl 12-oxo-6,9-dioxadispiro[2.1.45.33]dodecane-11-carboxylate

To a stirred solution 6,9-dioxadispiro[2.1.45.33]dodecan-12-one (1.2 g, 6.59 mmol) in Dimethyl carbonate (3 mL) was added sodium hydride 60% dispersion in mineral oil (757.01 mg, 32.93 mmol) in dimethyl carbonate (2 mL). Reaction mixture was stirred at room temperature for 5 min thereafter at 90° C. for 4 h. The reaction was monitored by the TLC and LCMS. After completion of reaction, reaction mixture was diluted with saturated solution of Ammonium chloride (20 mL), washed with ethyl acetate (3×20 mL). The combined organic layer was thoroughly washed with saturated brine, dried over sodium sulphate and concentrated under reduced pressure to afford to crude compound as Brown oily liquid (1.2 g), which was used further without purification LCMS; [M+H]⁺: 241.1.

### Step 4: Synthesis of methyldispirone

To a stirred solution of methyl 12-oxo-6,9-dioxadispiro[2.1.45.33]dodecane-11-carboxylate (1.2 g, 4.55 mmol) in sodium methoxide, 25% in methanol (2.46 g, 45.45 mmol, 2.53 mL), acetamidine hydrochloride, 97% (644.56 mg, 6.82 mmol) was added. Reaction mixture was stirred at 60° C. for 6 h. The reaction was monitored by TLC and LCMS. The reaction mixture acidified with 2N citric acid (10 mL) to pH (5-6) and washed with ethyl acetate (3×20 mL). The combined organic layer was washed with water followed by brine solution, dried over sodium sulphate, filter and concentrated under reduced pressure to get crude compound. Crude compound was purified by column chromatography, eluted with MeOH:DCM (1-10%) to get a required compound as a pale yellow solid (500 g, 44.31%), LCMS; [M+H]⁺: 249.17.

### Step 5: Synthesis of methyl 7-[5-chloranyl-2-[2-[methyl(oxidanylidene)dispiro-yl]ethoxy]phenyl]-5-methyl-thieno[3,2-b]pyridine-3-carboxylate

To a stirred solution of methyldispiro-one in DMSO (5 mL) was added Potassium carbonate, anhydrous, 99% (949.83 mg, 6.87 mmol) followed by methyl 7-[2-(2-bromanylethoxy)-5-chloranyl-phenyl]-5-methyl-thieno[3,2-b]pyridine-3-carboxylate (1.594 g, 3.6 mmol) was added and reaction mixture was heated to 60° C. for 4 h. Reaction was monitored by the TLC and LCMS. After completion of reaction, reaction mixture was quenched with water (10 mL) and washed with ethyl acetate (3×20 mL). The combined organic layer was washed with water followed by brine solution, dried over sodium sulphate, filter and concentrated under reduced pressure to get crude compound. Crude compound was purified by column chromatography, eluted with MeOH:DCM (1-5%) (650 mg, 38%), LCMS; [M+H]+: 608.48.

### Step 6: methyl 7-[5-chloranyl-2-[2-[2-methyl-4,6-bis(oxidanylidene)spiro[5,7-dihydroquinazoline-8,1′-cyclopropane]-3-yl]ethoxy]phenyl]-5-methyl-thieno[3,2-b]pyridine-3-carboxylate

To a stirred solution of methyl 7-[5-chloranyl-2-[2-[methyl(oxidanylidene)dispiro-yl]ethoxy]phenyl]-5-methyl-thieno[3,2-b]pyridine-3-carboxylate (650 mg, 1.06 mmol) in THF (2 mL) was added 4M HCl (3 mL) at room temperature. Reaction mixture was stirred at 60° C. for 6 h. Reaction was monitored by the TLC and LCMS. After completion of reaction mixture was basified with saturated NaHCO₃ solution (10 ml) and washed with Ethyl acetate (2×20 mL). The combined organic layer was washed with water, brine solution, dried over sodium sulphate and concentrated under reduced pressure to get crude material as yellow solid (450 mg, 30%), which was used further without purification LCMS; [M+H]⁺: 564.22.

### Step 7: methyl 7-[5-chloranyl-2-[2-[2-methyl-4-oxidanylidene-6-[4-[tris(fluorenyl)methoxy]-1-piperidyl]spiro[6,7-dihydro-5H-quinazoline-8,1′-cyclopropane]-3-yl]ethoxy]phenyl]-5-methyl-thieno[3,2-b]pyridine-3-carboxylate

To a solution chlorane; 4-[tris(fluorenyl)methoxy]piperidine (150. mg, 732.58 μmol) in DCE (1 mL), was added Triethylamine (4.40 mg, 43.48 μmol) at room temperature. Stirred the reaction mixture for 10 min followed by methyl 4-[5-chloranyl-2-[2-[2-methyl-4,6-bis(oxidanylidene)spiro[5,7-ihydroquinazoline-8,1′-cyclopropane]-3-yl]ethoxy]phenyl]-2-methyl-5H-cyclopenta[b]pyridine-7-carboxylate (200 mg, 366.29 μmol) was added and the resulting mixture was stirred for 16 h at room temperature. To this reaction mixture was added NaCNBH3 (1.45 g, 23.01 mmol) at room temperature and was stirred for 2 h at the same temperature. After completion of the reaction (monitored by TLC and LCMS), the reaction quenched with water (5 ml) and washed with 10% MeOH:DCM (2×20 ml) then washed with brine. The combined organic layer was dried over sodium sulphate and concentrated to afford methyl 7-[5-chloranyl-2-[2-[2-methyl-4-oxidanylidene-6-[4-[tris(fluorenyl)methoxy]-1-piperidyl]spiro[6,7-dihydro-5H-quinazoline-8,1′-cyclopropane]-3-yl]ethoxy]phenyl]-5-methyl-thieno[3,2-b]pyridine-3-carboxylate as a grey solid. (200 mg, 25.68%); LCMS; [M+H]+: 717.80.

### Step 8: 7-[5-chloranyl-2-[2-[(6S)-2-methyl-4-oxo-6-[4-(trifluoromethoxy)-1-piperidyl]spiro[6,7-dihydro-5H-quinazoline-8,1′-cyclopropane]-3-yl]ethoxy]phenyl]-5-methyl-thieno[3,2-b]pyridine-3-carboxylic acid and 7-[5-chloranyl-2-[2-[(6R)-2-methyl-4-oxo-6-[4-(trifluoromethoxy)-1-piperidyl]spiro[6,7-dihydro-5H-quinazoline-8,1′-cyclopropane]-3-yl]ethoxy]phenyl]-5-methyl-thieno[3,2-b]pyridine-3-carboxylic acid

To a solution methyl 7-[5-chloranyl-2-[2-[2-methyl-4-oxidanylidene-6-[4-[tris(fluorenyl)methoxy]-1-piperidyl]spiro[6,7-dihydro-5H-quinazoline-8,1′-cyclopropane]-3-yl]ethoxy]phenyl]-5-methyl-thieno[3,2-b]pyridine-3-carboxylate (200 mg, 278.86 μmol) in THE (0.7 mL) and water (0.3 ml), was added Lithium hydroxide monohydrate (29.26 mg, 697.16 mol) at room temperature. Resulting reaction mixture stirred for 4 h at room temperature. After completion of the reaction (monitored by TLC and LCMS), the reaction mixture was acidified (pH 5-6) with 2 N Citric acid and washed with 10% MeOH:DCM (2×30 mL) then washed with brine. The combined organic layer was dried over sodium sulphate and concentrated to afford for 7-(5-chloro-2-(2-(2′-methyl-4′-oxo-6′-(4-(trifluoromethoxy)piperidin-1-yl)-6′,7′-dihydro-4′H-spiro[cyclopropane-1,8′-quinazolin]-3′(5′H)-yl)ethoxy)phenyl)-5-methylthieno[3,2-b]pyridine-3-carboxylic acid as racemic mixture. This was purified by prep and pure fraction was concentrated to afford the racemic and subjected to SFC purification for separation of enantiomers by using below method.

Method: Column Chiralpak-IK (250×30×5μ), Co-solvent: 50% (30 mM Methanolic Ammonia in IPA), Column ovenTemp: Ambient, Diluent: 7.5 ml of IPA+CAN to afford separated enantiomer.

Compound 380: 7-[5-chloranyl-2-[2-[(6S)-2-methyl-4-oxo-6-[4-(trifluoromethoxy)-1-piperidyl]spiro[6,7-dihydro-5H-quinazoline-8,1′-cyclopropane]-3-yl]ethoxy]phenyl]-5-methyl-thieno[3,2-b]pyridine-3-carboxylic acid (29 mg, 14.69%).

LCMS: 717.80 [M+1].

HPLC: 99.36%,

Chiral HPLC: 99.72%, RT 7.16 min,

¹H NMR (DMSO-d6, 400 MHz): 8.74 (brs, 1H), 7.52 (dd, J=4.0, 8.0 Hz, 1H), 7.44 (d, J=4.0 Hz, 1H), 7.35 (s, 1H), 7.33 (d, J=8.0 Hz, 1H), 4.41 (t, J=4.0 Hz, 1H), 4.36 (t, J=8.0 Hz, 2H), 4.06 (t, J=8.0 Hz, 2H), 2.80-2.70 (m, 6H), 2.65-2.55 (m, 1H), 2.45-2.40 (m, 2H), 2.27-2.20 (m, 1H), 1.95-1.86 (m, 3H), 1.76-1.71 (m, 5H), 1.33-1.30 (m, 2H), 0.77-0.75 (m, 1H), 0.68-0.67 (m, 2H).

Compound 379: 7-[5-chloranyl-2-[2-[(6R)-2-methyl-4-oxo-6-[4-(trifluoromethoxy)-1-piperidyl]spiro[6,7-dihydro-5H-quinazoline-8,1′-cyclopropane]-3-yl]ethoxy]phenyl]-5-methyl-thieno[3,2-b]pyridine-3-carboxylic acid as a grey solid. (30.5 mg, 15.41%).

LCMS: 717.80 [M+1],

HPLC: 99.07%,

Chiral HPLC: 99.97%, RT 13.95 min,

¹H NMR (DMSO-d6, 400 MHz): 8.79 (brs, 1H), 7.52 (dd, J=4.0, 8.0 Hz), 1.7644 (d, J=4.0 Hz, 1H), 7.39 (s, 1H), 7.34 (d, J=8.0 Hz, 1H), 4.45-4.27 (m, 3H), 4.1-4.05 (m, 2H), 2.81-2.71 (m, 6H), 2.65-2.55 (m, 1H), 2.45-2.35 (m, 2H), 2.27-2.20 (m, 1H), 1.95-1.86 (m, 3H), 1.76-1.71 (m, 5H), 1.33-1.30 (m, 2H), 0.77-0.75 (m, 1H), 0.68-0.67 (m, 2H).

The following analogues are synthesized using above protocol from Int-1 (big keto) and the appropriate amine 2:

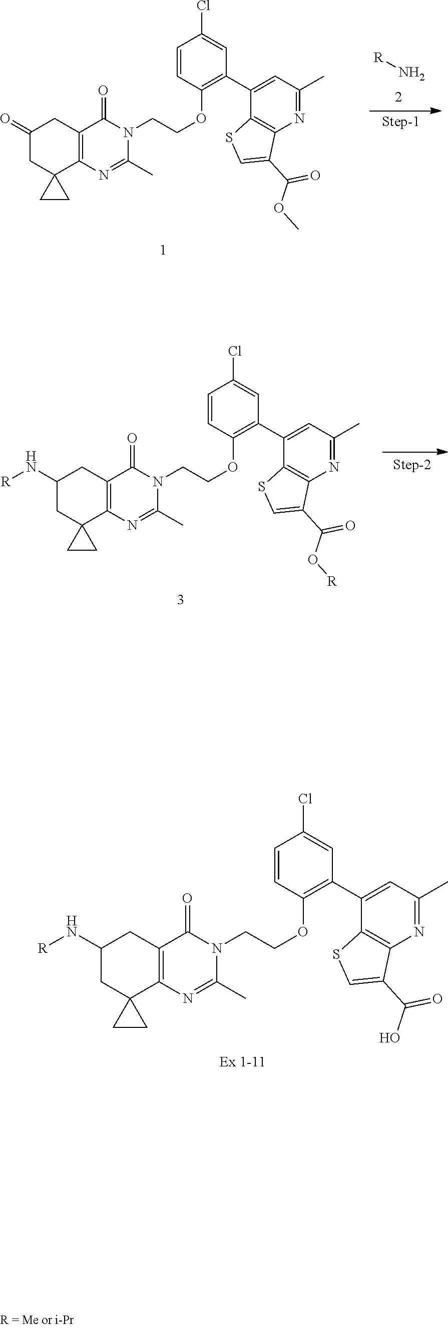

| | | | Method | |
|---|---|---|---|---|
| | | | of | LCMS |
| | | | prepa- | of 3: |
| Ex | Compound | Amines 2 | ration | [M + H]⁺ |
| 1 | Compound 544 Compound 545 | 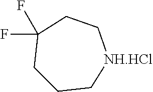 | A2 | 711.25 |
| 2 | Compound 591 Compound 564 | 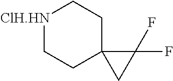 | A3 (2 h) | 723.2 |
| 3 | Compound 607 Compound 606 Compound | 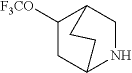 [SMILES: FC(F)(F)OC1CC2CCC1CN2] | A6 | 771.24 |
| | 605 | | | |
| | Compound | | | |
| | 604 | | | |
| 4 | Compound 619 Compound 618 | 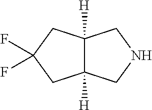 | A6 | 695.32 |
| 5 | Compound 561 Compound 560 | 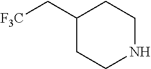 [SMILES: FC(F)(F)CC1CCNCC1] | A6 | 717.24 |
| 6 | Compound 603 Compound 602 | 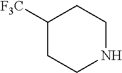 | A2 | 729.31 |
| 7 | Compound 151 Compound 372 Compound 371 | 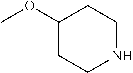 | A8 | 663.54 |
| 8 | Compound 610 Compound 609 Compound 617 Compound 616 | 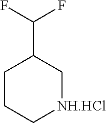 | A2 | 712.46 |
| 9 | Compound 583 Compound 582 Compound 581 Compound 580 | 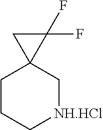 | A3 | 723.2 |
| 10 | Compound 613 Compound 611 | 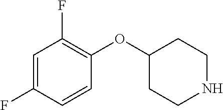 [SMILES: Fc1ccc(OC2CCNCC2)c(F)c1] | A4 | 789.29 |
| 11 | Compound 569 Compound 568 | 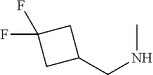 | A2 | 683.7 |

### 36. Experimental Procedure for Compound 579

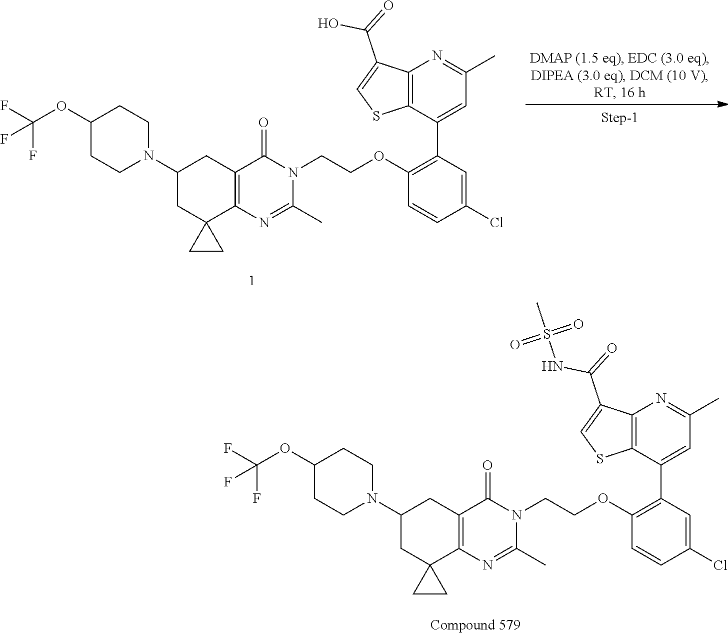 [SMILES: Cc1cc(-c2cc(Cl)ccc2OCCn2c(C)nc3c(c2=O)CC(N2CCC(OC(F)(F)F)CC2)CC32CC2)c2scc(C(=O)O)c2n1 ; Cc1cc(-c2cc(Cl)ccc2OCCn2c(C)nc3c(c2=O)CC(N2CCC(OC(F)(F)F)CC2)CC32CC2)c2scc(C(=O)NS(C)(=O)=O)c2n1]

### Step 1: Synthesis of 7-(5-chloro-2-(2-(2′-methyl-4′-oxo-6′-(4-(trifluoromethoxy)piperidin-1-yl)-6′,7′-dihydro-4′H-spiro[cyclopropane-1,8′-quinazolin]-3′(5′H)-yl)ethoxy)phenyl)-5-methyl-N-(methylsulfonyl)thieno[3,2-b]pyridine-3-carboxamide (Compound 579)

To a stirred solution acid compound (1 eq.) in DCM (10 V) at 0° C. were added methane sulfonamide (10 eq.), 4-Dimethylaminopyridine (1.5 eq.) and EDC (3 eq.), DIPEA (3.5 eq.). The resulting mixture was stirred at room temperature for 16 h. Progress of the reaction was monitored by LC-MS. After completion of the reaction, reaction mixture was diluted with water and washed with DCM (2×100 mL), the combined organic layers were dried over anhydrous Na₂SO₄, filtered and concentrated to get crude product. The crude product was purified by prep-HPLC, the required pure fractions were collected and concentrated to 7-(5-chloro-2-(2-(2′-methyl-4′-oxo-6′-(4-(trifluoromethoxy)piperidin-1-yl)-6′,7′-dihydro-4′H-spiro[cyclopropane-1,8′-quinazolin]-3′(5′H)-yl)ethoxy)phenyl)-5-methyl-N-(methylsulfonyl)thieno[3,2-b]pyridine-3-carboxamide.

¹H-NMR 400 MHz, DMSO-d₆: δ 8.90 (br s, 1H), 7.52 (dd, J=4.0 Hz, J=8.0 Hz, 1H), 7.44 (d, J=4.0 Hz, 1H), 7.39 (s, 1H), 7.34 (d, J=8.0 Hz, 1H), 4.45-4.27 (m, 3H), 4.1-4.05 (m, 2H), 2.81-2.71 (m, 6H), 2.65-2.55 (m, 1H), 2.45-2.35 (m, 2H), 2.27-2.20 (m, 1H), 2.01 (s, 3H) 1.95-1.86 (m, 3H), 1.76-1.71 (m, 5H), 1.33-1.30 (m, 2H), 0.77-0.75 (m, 1H), 0.68-0.67 (m, 2H).

### 37. Experimental Procedure for Compound 615, Compound 612, Compound 608, Compound 614

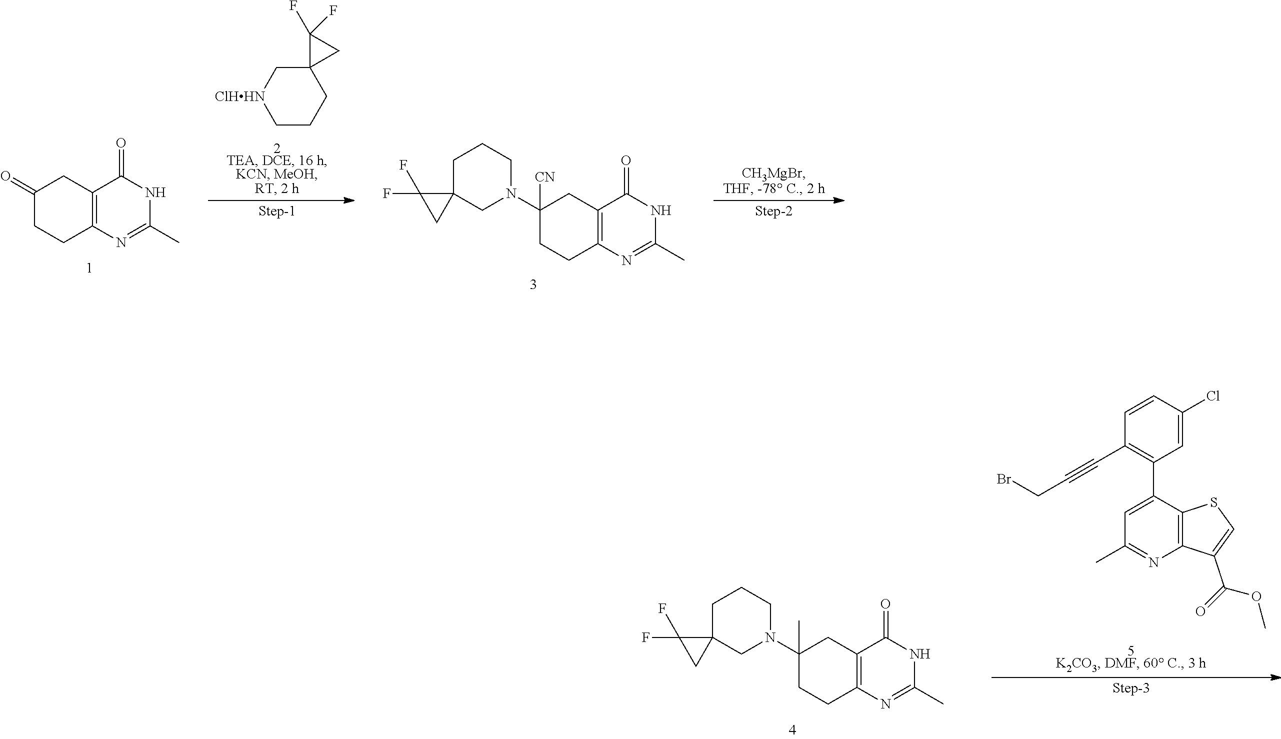

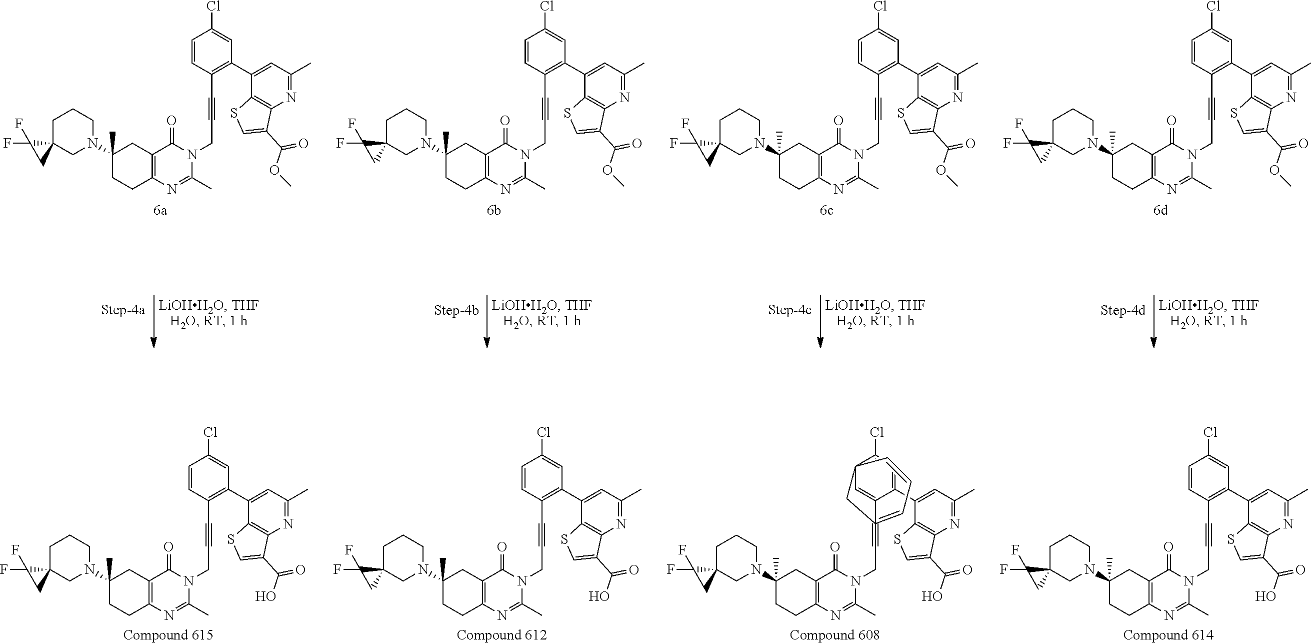

### Step-1: Synthesis of 6-(1,1-difluoro-5-azaspiro[2.5]octan-5-yl)-2-methyl-4-oxo-3,4,5,6,7,8-hexahydroquinazoline-6-carbonitrile (3)

Triethyl Amine (5.11 g, 50.51 mmol, 7.04 mL) was added to the solution of 2-methyl-3,5,7,8-tetra hydroquinazoline-4,6-dione (6 g, 33.67 mmol) and 1,1-difluoro-5-azaspiro[2.5]octane hydrochloride (9.27 g, 50.51 mmol) in DCE (100 mL) at room temperature and stirred for 3 h. Reaction mixture was cooled to 0° C. and KCN (1.99 g, 28.81 mmol) was added portion wise and allowed to warm at room temperature for 5 h. The reaction mixture was cooled to 0° C. and quenched with water and extract with DCM (500 mL×3). The combined organic layer was washed with cold water (500 mL) and brine solution (500 mL), the organic layer was dried over Na₂SO₄ and filtered. The filtrate was concentrated under reduced pressure to afford 6-(1,1-difluoro-5-azaspiro[2.5]octan-5-yl)-2-methyl-4-oxo-3,4,5,6,7,8-hexahydroquinazoline-6-carbonitrile (8 g, 22%) as light yellow solid. LCMS; 335.1 [M+1].

### Step-2: 6-(1,1-difluoro-5-azaspiro[2.S]octan-5-yl)-2,6-dimethyl-S, 6,7,8-tetrahydroquinazolin-4(3H)-one (4)

Methyl Magnesium Bromide (80 mL, 11.23 mmol) was added slowly dropwise to the solution of 6-[2,2-bis(fluorenyl)-5-azaspiro[2.5]octan-5-yl]-2-methyl-4-oxidanylidene-3,5,7,8-tetrahydroquinazoline-6-carbonitrile (3.6 g, 10.77 mmol) in THF (35 mL) at −78° C. and the resulting reaction mixture allowed to room temperature and stirred for 2 h. The reaction mixture was cooled to 0° C. and quenched with sat. aq. ammonium chloride solution. Then extract with ethyl acetate (300 mL×3). The combined organic layer was washed with water (300 mL) and brine (300 mL), the organic layer was dried over Na₂SO₄ and filtered. The filtrate was concentrated under reduced pressure to afford crude compound. This was purified by combi-flash column chromatography using 230-400 mesh silica gel and the desired product was eluted at 0-10% of MeOH in DCM as the eluent. Pure fractions (determined by TLC) were combined and evaporated under reduced pressure to afford 6-(1,1-difluoro-5-azaspiro[2.5]octan-5-yl)-2,6-dimethyl-5,6,7,8-tetrahydroquinazolin-4(3H)-one (2.5 g, 59%) as a brown solid. LCMS; 324.10[M+1].

### Step 3: methyl 7-(5-chloro-2-(3-((S)-6-((S)-1,1-difluoro-5-azaspiro[2.5]octan-5-yl)-2,6-dimethyl-4-oxo-5,6,7,8-tetrahydroquinazolin-3(4H)-yl)prop-1-yn-1-yl)phenyl)-5-methylthieno[3,2-b]pyridine-3-carboxylate (6)

Potassium carbonate (384.07 mg, 2.78 mmol) was added to the solution of 6-[2,2-bis(fluorenyl)-5-azaspiro[2.5]octan-5-yl]-2,6-di(methyl)-3,5,7,8-tetrahydroquinazolin-4-one (300 mg, 927.70 μmol) in DMF (5 mL) at room temperature and slowly heated to 75° C. and stirred for 15 min. Then methyl 7-(2-(3-bromoprop-1-yn-1-yl)-5-chlorophenyl)-5-methylthieno[3,2-b]pyridine-3-carboxylate (434 mg, 834.20 μmol) was added slowly portion wise to the reaction mixture at 75° C. and the resulting reaction mixture was stirred for another 4 h at same temperature. The reaction mixture was cooled to room temperature and quenched the water and extract with ethyl acetate (100 mL×3). The combined organic layer was washed with water (100 mL) and brine (100 mL), the organic layer was dried over Na₂SO₄ and filtered. The filtrate was concentrated under reduced pressure to afford crude as brown liquid. The crude was purified by combi-flash column chromatography using 230-400 mesh silica gel and the desired product was eluted at 0-10% of MeOH in DCM as the eluent. Pure fractions (determined by TLC) were combined and evaporated under reduced pressure to afford methyl 7-(5-chloro-2-(3-((S)-6-((S)-1,1-difluoro-5-azaspiro[2.5]octan-5-yl)-2,6-dimethyl-4-oxo-5,6,7,8-tetrahydroquinazolin-3(4H)-yl)prop-1-yn-1-yl)phenyl)-5-methylthieno[3,2-b]pyridine-3-carboxylate (120 mg, 27%) as an off white solid. LCMS; [M+H]: 677.18

### Step 4: 7-(5-chloro-2-(3-((S)-6-((S)-1,1-difluoro-5-azaspiro[2.5]octan-5-yl)-2,6-dimethyl-4-oxo-5,6,7,8-tetrahydroquinazolin-3(4H)-yl)prop-1-yn-1-yl)phenyl)-5-methylthieno[3,2-b]pyridine-3-carboxylic acid (Compound 615)

Lithium hydroxide monohydrate (13.94 mg, 332.25 μmol, 9.23 μL) was added to the solution of methyl 7-[5-chloranyl-2-[3-[(3R,6S)-6-[(3S)-2,2-difluoro-5-azaspiro[2.5]octan-5-yl]-2,6-di(methyl)-4-oxidanylidene-7,8-dihydro-5H-quinazolin-3-yl]prop-1-ynyl]phenyl]-5-methyl-thieno[3,2-b]pyridine-3-carboxylate (45 mg, 66.45 μmol) in a mixture of solvents THE and H₂O (2 mL & 0.5 mL) at room temperature. The resulting mixture was stirred at room temperature for 2 h. The resulting reaction mixture was concentrated at 45° C. under reduced pressure. This reaction mass was diluted in water and acidified with saturated aqueous citric acid solution to pH 4-6. Then washed 10% MeOH in Dichloromethane (30 mL×2). The combined organic layer was concentrated under reduced pressure to obtain the crude material. This was purified by Prep-HPLC Column: YMC-TRIART-C18 (250*10 mm), 5u MCL-ANL-COL-2023-017 Mobile phase: 10 mM Ammonium Bi Carbonate in H₂O:ACETONITRILE GRADIENT: (T % B): 0/15, 7/50, 11/58, 11.1/98, 14/98, 14.1/15, 17/15 Flow Rate: 8 ml/min Diluent: ACN+H₂O to afford desired product as an off white solid. Isomer were separated to SFC Method Conditions: Column: CHIRALCEL OD-H (4.6*250 mm)₅ μm Co-solvent: 0.5% MEONH3 IN MeOH Total flow: 3 mL/min % of CO₂: 60% of Co-Solvent: 40 ABPR: 1500 psi Temperature: 30° C. to afford two desired isomers (1138, 1139, 1140 and 1141) as an off white solid. LCMS: 663.3 [M+1].

TABLE E2
NMR data

| Compound | |
|---|---|
| No. | 1NMR |
| 79 | 1H-NMR: (400 MHz, DMSO-d6): δ 13.03 (s, 1H), 9.00 (d, J = 1.60 Hz, 1H), |
| | 7.60 (dd, J = 2.80, 9.00 Hz, 1H), 7.52 (s, 1H), 7.45-7.44 (m, 1H), 7.35 (d, J = |
| | 8.80 Hz, 1H), 4.38-4.34 (m, 2H), 4.12 (t, J = 4.40 Hz, 2H), 3.60-3.44 (m, 6H), |
| | 3.29-3.28 (m, 4H), 3.07-3.04 (m, 1H), 2.93-2.89 (m, 1H), 2.75 (s, 3H), 2.61- |
| | 2.56 (m, 2H), 2.36-2.33 (m, 1H), 2.18 (d, J = 12.00 Hz, 1H), 2.09-2.06 (m, 3H), |
| | 1.85-1.851(m, 2H), 1.77 (s, 3H). |
| 100 | 1H-NMR: (400 MHz, DMSO-d6): δ 7.29 (t, J = 7.60 Hz, 2H), 5.97 (td, J = 4.80, |
| | 52.00 Hz, 1H), 4.30 (t, J = 4.40 Hz, 2H), 4.08 (d, J = 4.40 Hz, 2H), 3.26-2.90 |
| | (m, 2H), 2.77-2.60 (m, 5H), 2.50-2.42 (m, 4H), 2.33-2.15 (m, 4H), 2.15-1.84 |
| | (m, 2H), 1.70-1.18 (m, 8H). |
| 101 | 1H-NMR: (400 MHz, DMSO-d6): δ 8.40 (s, 1H), 7.55 (dd, J = 2.40, 9.00 Hz, |
| | 1H), 7.40 (d, J = 2.80 Hz, 1H), 7.25-7.31 (m, 2H), 5.96 (td, J = 5.20, 56.80 Hz, |
| | 1H), 4.30 (d, J = 4.40 Hz, 2H), 4.08 (d, J = 4.40 Hz, 2H), 2.83-2.85 (m, 2H), |
| | 2.68-2.46 (m, 7H), 2.23-2.15 (m, 3H), 1.97-1.86 (m, 2H), 1.69-1.44 (m, 7H), |
| | 1.46-1.23 (m, 3H) |
| 102 | 1H-NMR: (400 MHz, DMSO-d6): δ 13.31 (br s, 1H), 8.68 (br s, 1 H), 7.57 |
| | (dd, J = 4 Hz, J = 8 Hz, 1H), 7.44 (d, J = 4 Hz, 1H), 7.35-7.30 (m, 2H), 4.30 |
| | (t, J = 4 Hz, 2H), 4.07 (t, J = 4 Hz, 2H), 3.22 (s, 3H), 3.17-3.13 (m, 1H), 3.00 |
| | (t, J = 4 Hz, 1H), 2.87 (t, J = 4 Hz, 2H), 2.71 (s, 3H), 2.45-2.43 (m, 2H), 2.13 |
| | (t, J = 8 Hz, 3H), 1.96-1.94 (m, 1H), 1.87-1.84 (m, 2H), 1.62 (s, 3H), 1.53-1.48 |
| | (m, 1H), 1.43-1.37 (m, 2H), 0.89 (d, J = 8 Hz, 3H). |
| 103 | 1H-NMR: (400 MHz, DMSO-d6): δ 13.28 (br s, 1H), 8.68 (br s, 1 H), 7.57 |
| | (dd, J = 4 Hz, J = 8 Hz, 1H), 7.44 (d, J = 4 Hz, 1H), 7.38 (s, 1H), 7.33 (d, J = 8 |
| | Hz, 1H), 4.38 (t, J = 4 Hz, 2H), 4.07 (t, J = 4 Hz, 2H), 3.18 (s, 3H), 3.17-3.13 |
| | (m, 1H), 3.00 (t, J = 4 Hz, 1H), 2.86 (t, J = 4 Hz, 2H), 2.72 (s, 3H), 2.44-2.39 |
| | (m, 2H), 2.13 (t, J = 8 Hz, 3H), 1.96-1.94 (m, 1H), 1.87-1.84 (m, 2H), 1.61 (s, |
| | 3H), 1.53-1.48 (m, 1H), 1.43-1.35 (m, 2H), 0.89 (d, J = 8 Hz, 3H). |
| 104 | 1H-NMR: (400 MHz, DMSO-d6): δ 8.90 (s, 1H), 7.58 (dd, J = 2.80, 8.80 Hz, |
| | 1H), 7.43 (d, J = 2.80 Hz, 2H), 7.34 (d, J = 8.80 Hz, 1H), 4.42-4.47 (m, 1H), |
| | 4.32 (t, J = 5.60 Hz, 2H), 4.06-4.08 (m, 2H), 3.45 (s, 3H), 2.83-2.86 (m, 2H), |
| | 2.73 (s, 3H), 2.60 (m, 1H), 2.40-2.50 (m, 5H), 2.15-2.22 (m, 1H), 1.86-1.96 (m, |
| | 3H), 1.67-1.71 (m, 2H), 1.53-1.61 (m, 3H), 1.18-1.24 (m, 1H) |
| 105 | 1H-NMR: (400 MHz, DMSO-d6): δ 8.92 (s, 1H), 7.58 (dd, J = 2.80, 9.00 Hz, |
| | 1H), 7.43 (d, J = 2.80 Hz, 2H), 7.34 (d, J = 8.80 Hz, 1H), 4.43-4.45 (m, 2H), |
| | 4.08 (d, J = 4.80 Hz, 2H), 3.45 (s, 3H), 2.72-2.84 (m, 2H), 2.50-2.51 (m, 3H), |
| | 2.50-2.51 (m, 1H), 2.44-2.50 (m, 5H), 2.18-2.22 (m, 1H), 1.91-1.96 (m, 3H), |
| | 1.71-1.86 (m, 2H), 1.58-1.67 (m, 4H), 1.24-1.07 (m, 1H) |
| 106 | 1H-NMR: (400 MHz, DMSO-d6): δ 8.74 (s, 1H), 7.57 (dd, J = 2.40, 9.00 Hz, |
| | 1H), 7.44 (d, J = 2.80 Hz, 1H), 7.39 (s, 1H), 7.32 (d, J = 8.80 Hz, 1H), 4.42-4.32 |
| | (m, 2H), 4.07-4.08 (m, 2H), 2.85-2.93 (m, 2H), 2.72 (s, 3H), 2.46-2.50 (m, 4H), |
| | 2.25-2.13 (m, 5H), 1.88-1.86 (m, 1H), 1.72 (d, J = 12.40 Hz, 2H), 1.59 (s, 3H), |
| | 1.59-1.56 (m, 2H), 1.29-1.24 (m, 2H) |
| 107 | 1H-NMR: (400 MHz, DMSO-d6): 8.69 (s, 1H), 7.56 (dd, J = 15.60, 7.60 Hz, |
| | 1H), 7.44 (s, 1H), 7.37 (s, 1H), 7.32 (d, J = 8.80 Hz, 1H), 4.32 (m, 2H), 4.08- |
| | 4.08 (m, 2H), 3.00-2.88 (m, 2H), 2.71 (s, 3H), 2.54-2.53 (m, 1H), 2.47 (m, 3H), |
| | 2.30-2.14 (m, 5H), 1.95-1.92 (m, 1H), 1.76-1.73 (m, 2H), 1.59 (s, 3H), 1.56 (m, |
| | 2H) 1.29-1.24 (m, 2H) |
| 108 | 1H-NMR: (400 MHz, DMSO-d6):: 8.54 (s, 1H), 8.04 (s, 1H), 7.75-7.54 (m, 1H), |
| | 7.51 (s, 1H), 7.42 (d, J = 2.80 Hz, 1H), 7.32-7.30 (m, 2H), 4.31-4.30 (m, 2H), |
| | 4.15-4.08 (m, 2H), 3.06-2.95 (m, 1H), 2.70 (s, 3H), 2.68-2.62 (m, 3H), 2.51- |
| | 2.49 (m, 2H), 2.40-2.31 (m, 1H), 2.25-2.19 (m, 1H), 1.91-1.90 (m, 1H), 1.84- |
| | 1.75 (m, 3H), 1.62 (s, 3H), 1.11-1.06 (m, 3H), 1.04-1.01 (m, 1H) |
| 109 | 1H-NMR: (400 MHz, DMSO-d6):: 8.52 (s, 1H), 8.04 (s, 1H), 7.57-7.54 (m, 1H), |
| | 7.51 (s, 1H), 7.36 (d, J = 9.20 Hz, 1H), 6.83 (d, J = 1.2 Hz, 1H), 7.42-7.41 (m, |
| | 1H), 7.33-7.30 (m, 2H), 4.31-4.30 (m, 2H), 4.13-4.08 (m, 3H), 3.05-2.95 (m, |
| | 2H), 2.691 (s, 3H), 2.51-2.49 (m, 2H), 2.39-2.31 (m, 2H), 2.01-1.98 (m, 2H), |
| | 1.91-1.86 (m, 2H), 1.629 (s, 3H), 1.07-1.03 (m, 3H), |
| 110 | 1H-NMR: (400 MHz, DMSO-d6): 8.74 (s, 1H), 7.57 (dd, J = 2.40, 9.00 Hz, 2H), |
| | 7.43 (d, J = 2.40 Hz, 1H), 7.39 (s, 1H), 7.32 (d, J = 9.20 Hz, 1H), 4.32 (d, J = |
| | 4.80 Hz, 2H), 4.07 (d, J = 3.60 Hz, 2H), 3.49-3.55 (m, 4H), 2.79-2.72 (m, 2H), |
| | 2.67 (s, 3H), 2.42-2.46 (m, 2H), 2.23 (m, 2H), 1.67-1.64 (m, 2H), 1.59 (s, 3H), |
| | 1.36 (s, 3H), 1.22-1.23 (m, 2H), |
| 111 | 1H-NMR: (400 MHz, (DMSO-d6): 8.80 (s, 1H), 7.58 (dd, J = -2.40, -9.00 Hz, |
| | 1H), 7.43 (dd, J = 6.80, Hz, 2H), 7.42 (d, J = 0.00 Hz, 1H), 4.31-4.34 (m, 2H), |
| | 4.06-4.03 (m, 2H), 3.55-3.33 (m, 4H), 2.89-2.80 (m, 2H), 2.72 (s, 3H), 2.71- |
| | 2.65 (m, 1H), 2.47 (s, 3H), 2.34-2.16 (m, 4H), 1.93-1.87 (m, 1H), 1.577(s, 6H) |
| | 1.71 (d, J = 10.40 Hz, 2H), |
| 112 | 1H-NMR: (400 MHz, DMSO-d6): δ 8.72 (s, 1H), 8.07 (d, J = 5.20 Hz, 1H), |
| | 7.58 (dd, J = 2.40, 9.00 Hz, 1H), 7.44 (d, J = 2.40 Hz, 1H), 7.41 (s, 1H), 7.32 |
| | (d, J = 8.80 Hz, 1H), 6.97 (d, J = 5.20 Hz, 1H), 6.81 (s, 1H), 6.13-5.85 (m, 1H), |
| | 4.36-4.27 (m, 2H), 4.07-4.02 (m, 2H), 3.88-3.81 (m, 2H), 3.78 (s, 3H), 3.03- |
| | 2.96 (m, 2H), 2.72 (s, 3H), 2.68-2.67 (m, 1H), 2.55-2.51 (m, 1H), 2.50-2.45 (m, |
| | 2H), 2.22-2.16 (m, 1H), 1.91-1.89 (m, 1H), 1.66-1.62 (m, 1H), 1.55 (s, 3H). |
| 113 | 1H-NMR: (400 MHz, DMSO-d6): δ 8.39 (s, 1H), 7.54 (dd, J = 2.40, 8.80 Hz, |
| | 1H), 7.40 (d, J = 2.40 Hz, 1H), 7.29 (d, J = 9.20 Hz, 1H), 7.22 (s, 1H), 4.30 (t, |
| | J = 4.0 Hz, 2H), 4.08 (t, J = 4.0 Hz, 2H), 3.22 (m, 1H), 2.85-2.87 (m, 1H), 2.68 |
| | (s, 3H), 2.60-2.66 (m, 3H), 2.41-2.50 (m, 3H), 2.20-2.33 (m, 2H), 1.88-1.96 (m, |
| | 4H), 1.80-1.83 (m, 1H), 1.57 (s, 3H), 1.43-1.48 (m, 1H), 1.23 (s, 1H). |
| 114 | 1H-NMR: (400 MHz, DMSO-d6): 8.78 (s, 1H), 7.58 (dd, J = 4.00, 8.00 Hz, 1H), |
| | 7.44-7.40 (m, 2H), 7.33-7.31 (m, 1H), 4.33-4.31 (m, 2H), 4.08-4.06 (m, 2H), |
| | 3.33 (s, 3H), 3.16-3.08 (m, 1H), 2.85-2.82 (m, 1H), 2.73 (s, 3H), 2.59-2.51 (m, |
| | 2H), 2.33-2.07 (m, 5H), 1.88-1.76 (m, 3H), 1.64 (s, 3H), 1.62-1.56 (m, 1H), |
| | 1.37-1.23 (m, 2H), 0.96 (s, 3H) |
| 115 | 1H-NMR: (400 MHz, DMSO-d6):: δ 8.72 (s, 1H), 7.57 (dd, J = 2.40, 8.80 Hz, |
| | 1H), 7.38-7.43 (m, 2H), 7.32 (d, J = 8.80 Hz, 1H), 4.32 (m, 2H), 4.07 (m, 2H), |
| | 2.98-3.09 (m, 5H), 2.73 (s, 3H), 2.61 (m, 1H), 2.47-2.36 (m, 3H), 2.32-2.33 (m, |
| | 3H), 2.00-1.97 (m, 2H), 1.85-1.87 (m, 1H), 1.59 (m, 6H), 1.21 (t, J = 7.60 Hz, 3H). |
| 116 | 1H-NMR: (400 MHz, DMSO-d6): δ 8.75 (s, 1H), 7.58 (dd, J = 2.80, 8.80 Hz, |
| | 1H), 7.31-7.44 (m, 3H), 4.32 (t, J = 5.60 Hz, 2H), 4.07 (t, J = 4.80 Hz, 2H), |
| | 2.44-2.87 (m, 9H), 2.15-2.24 (m, 3H), 1.86-1.97 (m, 2H), 1.71-1.24 (m, 9H). |
| 117 | 1H-NMR: (400 MHz, DMSO-d6): δ 8.78 (s, 1H), 7.58 (dd, J = 2.40, 9.00 Hz, |
| | 1H), 7.32-7.44 (m, 3H), 5.97 (td, J = 4.80, 56.80 Hz, 1H), 4.32 (br s, 2H), 4.07 |
| | (t, J = 4.80 Hz, 2H), 2.91 (d, J = 8.40 Hz, 1H), 2.78-2.44 (m, 8H), 2.28-2.15 (m, |
| | 4H), 2.09-1.84 (m, 2H), 1.71-1.41 (m, 5H), 1.25-1.21 (m, 2H). |
| 118 | 1H-NMR 400 MHz, DMSO-d6): δ 8.69 (d, J = 5.20 Hz, 1H), 7.78 (s, 1H), |
| | 7.64 (s, 1H), 7.56-7.52 (m, 1H), 7.49-7.48 (m, 1H), 7.27 (d, J = 9.20 Hz, 1H), |
| | 4.33 (s, 3H), 4.03 (s, 2H), 3.57 (s, 1H), 3.53 (m, 1H), 3.47-3.45 (m, 1H), 3.39- |
| | 3.38 (m, 1H), 2.88-2.67 (m, 8H), 2.61-2.52 (m, 2H), 2.33-2.31 (m, 4H), 1.80- |
| | 1.78 (m, 1H), 1.61 (s, 2H). |
| 119 | 1H-NMR: (400 MHz, DMSO-d6): δ: 13.44 (brs, 1H), 8.72 (s, 1H), 7.59 (dd, J = |
| | 2.8 Hz, 9.0 Hz, 1H), 7.43-7.41 (m, 2H), 7.33-7.31 (m, 1H), 4.34 (t, J = 4.8 Hz, |
| | 2H), 4.07 (t, J = 4.8 Hz, 2H), 3.42-3.31 (m, 2H), 3.25-3.19 (m, |
| | 2H), 2.73 (s, 3H), 2.66-2.57 (m, 1H), 2.55-2.51 (m, 1H), 2.50- |
| | 2.49 (m, 2H), 2.22-2.15 (m, 1H), 1.97-1.90 (m, 1H), 1.63-1.57 (m, 4H). |
| 120 | 1H-NMR: (400 MHz, DMSO-d6): δ 13.60 (s, 1H), 8.82 (s, 1H), 7.58 (dd, J = |
| | 2.40, 9.00 Hz, 1H), 7.44 (d, J = 1.60 Hz, 2H), 7.33 (d, J = 8.80 Hz, 1H), 5.96 |
| | (ddd, J = 5.20, 57.00, Hz, 1H), 4.34 (t, J = 4.80 Hz, 2H), 4.07 (t, J = 4.80 Hz, |
| | 2H), 2.74 (s, 3H), 2.66-2.69 (m, 2H), 2.55-2.58 (m, 3H), 2.47-2.50 (m, 1H), |
| | 2.29-2.36 (m, 3H), 1.91 (m, 2H), 1.63-1.68 (m, 2H), 1.57 (s, 3H), 1.24 (s, 1H). |
| 121 | 1H-NMR: (400 MHz, DMSO-d6): δ 8.81 (s, 1H), 7.58 (dd, J = 4.00, 10.00 Hz, |
| | 1H), 7.44 (d, J = 4.00 Hz, 2H), 7.33 (d, J = 8.00 Hz, 1H), 5.97 (ddd, J = 4.00, |
| | 56.00, Hz, 1H), 4.34 (t, J = 4.00 Hz, 2H), 4.08 (t, J = 4.00 Hz, 2H), 2.80-2.85 |
| | (m, 1H), 2.73 (s, 3H), 2.51-2.66 (m, 6H), 2.33-2.38 (m, 2H), 2.21-2.27 (m, 1H), |
| | 1.85-1.93 (m, 2H), 1.62-1.72 (m, 2H), 1.57 (s, 3H), 1.24 (s, 1H). |
| 122 | 1H-NMR: (400 MHz, DMSO-d6): δ 8.60 (s, 1H), 7.56 (dd, J = 0.00, 10.00 Hz, |
| | 1H), 7.42 (s, 1H), 7.34 (d, J = 4.00 Hz, 1H), 7.30 (s, 1H), 5.96 (ddd, J = 8.00, |
| | 58.00, Hz, 1H), 4.32 (t, J = 4.00 Hz, 2H), 4.08 (t, J = 4.00 Hz, 2H), 2.64-2.71 |
| | (m, 5H), 2.51-2.57 (m, 5H), 2.35-2.39 (m, 2H), 2.22-2.28 (m, 1H), 1.84-1.93 |
| | (m, 2H), 1.61-1.68 (m, 5H), 1.23 (s, 1H). |
| 123 | 1H-NMR: (400 MHz, DMSO-d6): δ 8.66 (br s, 1H), 8.34 (br s, 1H), 7.57 |
| | (dd, J = 4.0, 8.0 Hz, 1H), 7.43 (s, 1H), 7.36-7.31 (m, 2H), 4.35-4.26 (m, 2H), |
| | 4.10-4.01 (m, 2H), 2.71-2.66 (m, 4H), 2.49-2.44 (m, 2H), 1.77-1.71 (m, 1H), |
| | 1.60 (s, 4H), 1.27 (s, 3H), 1.23 (s, 1H), 1.13 (s, 3H). |
| 124 | 1H-NMR: (400 MHz, DMSO-d6): 8.77 (s, 1H), 8.29 (s, 1H), 7.59-7.56 (dd, J = |
| | 2.4 Hz, 8.8 Hz, 1H), 7.44-7.40 (m, 1H), 7.32 (d, J = 8.8 Hz, 1H), 4.38-4.25 (m, |
| | 2H), 4.12-4.04 (m, 2H), 3.02-2.92 (m, 2H), 2.73 (s, 3H), 2.59-2.58 (m, 3H), |
| | 2.48-2.39 (m, 4H), 2.23-2.14 (m, 3H), 1.87-1.58 (m, 8H), 1.37-1.31 (m, 2H). |
| 125 | 1H-NMR 400 MHz, DMSO-d6): 8.75 (s, 1H), 8.07 (d, J = 5.20 Hz, 1H), 7.58 |
| | (dd, J = 2.80, 8.80 Hz, 1H), 7.44-7.41(m, 2H), 7.33(d, J = 8.8 Hz, 1H), 6.95 (d, |
| | J = 0.80 Hz, 1H), 6.76 (s, 1H), 6.57 (s, 1H), 4.32-4.29 (m, 2H), 4.08- |
| | 4.07(m, 2H), 3.82 (s, 3H), 3.6-3.5(m, 2H) , 2.73 (s, 3H), 2.68-2.46 (m, 3H), 2.33- |
| | 2.32 (m, 1H), 1.95-1.90 (m, 1H), 1.62 (s, 3H), 1.66-1.59 (m, 1H). |
| 126 | 1H-NMR: (400 MHz, DMSO-d6): δ 13.50 (br s, 1H) 8.74 ( s, 1H), 7.58 (dd, J = |
| | 4.0, 8.0 Hz, 1H), 7.44-7.44 (m, 2H), 7.33 (d, J = 12 Hz, 1H), 4.31 (t, J = 4.0 Hz, |
| | 2H), 4.07 (t, J = 12 Hz, 2H), 3.66 (s, 1H), 3.59 (s, 1H), 3.73 (s, 4H), 2.61-2.35 |
| | (m, 3H), 2.20-2.06 (m, 3H), 1.98-1.80 (m, 5H), 1.70-1.54 (m, 2H). 1.57-1.50 |
| | (m, 4H). |
| 127 | 1H-NMR: 400 MHz CDCl3): 8.69 (s, 1H), 7.46-7.40 (m, 3H), 7.29-7.28 (m, |
| | 1H), 7.21-7.20 (m, 1H), 7.04-7.01 (m, 1H), 4.39-4.35 (m, 2H), 4.19-4.15 (m, |
| | 2H), 3.17-3.16 (m, 3H), 2.81 (s, 3H), 2.80-2.56 (m, 5H), 2.34-2.44 (m, 3H), |
| | 2.15-2.11 (m, 1H), 1.92-1.87(m, 5H), 1.84-1.83 (m, 1H), 1.81-1.63 (m, 3H), |
| | 1.25 (s, 1H), |
| 128 | 1H-NMR: (400 MHz, DMSO-d6): δ 8.74 (s, 1H), 8.39 (s, 2H), 7.57 (dd, J = |
| | 4.00, 8.00 Hz, 1H), 7.43 (d, J = 4.00 Hz, 1H), 7.37 (s, 1H), 7.32 (d, J = 8.00 Hz, |
| | 1H), 4.33 (t, J = 4.00 Hz, 2H), 4.07 (t, J = 8.00 Hz, 2H), 3.02 (d, J = 8.00 Hz, |
| | 2H), 2.97 (d, J = 4.00 Hz, 2H), 2.79-2.74 (m, 6H), 2.71 (s, 3H), 2.45-2.39 (m, |
| | 2H), 2.30-2.25 (m, 1H), 1.81-1.60 (m, 3H), 1.60 (s, 3H), 1.51-1.45 (m, 1H), |
| 129 | 1H-NMR: 400 MHz, DMSO-d6): 8.66 (s, 1H), 8.054 (dd, J = 5.20, Hz, 1H), |
| | 7.58-7.57 (m, 1H), 7.55(d, J = 2.80 Hz, 1H), 7.37 (s, 1H), 7.31 (d, J = 8.80 Hz, |
| | 1H), 6.96 (d, J = 5.2 Hz, 1H), 6.79 (s, 1H), 4.33-4.29(m, 2H), 4.06-4.04 (m, |
| | 2H), 3.81 (s, 3H), 3.71-3.64 (m, 2H), 2.81-2.80 (m, 1H), 2.71-2.68 (m, 3H), |
| | 2.60-2.55 (m, 2H), 2.51- 2.49 (m, 3H), 2.33-2.32 (m, 1H), 1.89-1.88 (m, 1H), |
| | 1.61-1.59(m, 4H), 0.98-0.95(m, 3H), |
| 130 | 1H-NMR: (400 MHz, DMSO-d6): 7.57 (dd, J = 2.40, 8.40 Hz, 1H), 7.44-7.43 |
| | (m, 1H), 7.39 (s, 1H), 7.34 (s, 1H), 7.31-7.30 (m, 1H), 6.94 (dd, J = 3.60, 5.00 |
| | Hz, 1H), 6.88-6.87 (m, 1H), 4.33 (m, 2H), 4.09-4.08 (m, 2H), 3.39-3.02 (m, |
| | 2H), 2.97-2.78 (m, 2H), 2.68 (s, 3H), 2.61-2.58 (m, 1H), 2.53-2.47 (m, 2H), |
| | 2.36-2.24 (m, 2H), 1.97-1.88 (m, 3H), 1.88 (s, 3H), 1.60-1.57 (m, 3H), 1.14- |
| | 1.10 (m, 2H), |
| 131 | 1H-NMR: (400 MHz, DMSO-d6): δ 8.52 (s, 1H), 7.55 (dd, J = 0.00, 8.00 Hz, |
| | 1H), 7.41 (d, J = 0.00 Hz, 1H), 7.31-7.28 (m, 2H), 6.88 (s, 1H), 4.31 (t, J = 4.00 |
| | Hz, 2H), 4.07 (t, J = 4.00 Hz, 2H), 3.01 (d, J = 4.00 Hz, 2H), 2.96-2.94 (d, J = |
| | 8.00 Hz, 2H), 2.69 (t, J = Hz, 3H), 2.62-2.57 (m, 2H), 2.49-2.40 (m, 2H), 2.33- |
| | 2.31 (m, 1H), 1.97 (dd, J = 8.00, 16.00 Hz, 1H), 1.88-1.66 (m, 7H), 1.63 (s, |
| | 3H), 1.48-1.44 (m, 1H), |
| 132 | 1H-NMR: (400 MHz, DMSO-d6): δ 8.82 (s, 1H), 7.58 (dd, J = 2.80, 8.80 Hz, |
| | 1H), 7.42-7.44 (m, 2H), 7.33 (d, J = 9.20 Hz, 1H), 7.25-7.29 (m, 2H), 7.12-7.06 |
| | (m, 2H), 4.33 (d, J = 6.80 Hz, 2H), 4.08 (d, J = 4.00 Hz, 2H), 3.17-3.01 (m, 3H), |
| | 2.99-2.49 (m, 7H), 2.35-2.27 (m, 3H), 1.93-1.59 (m, 9H). |
| 133 | 1H-NMR: (400 MHz, DMSO-d6): δ 9.30 (d, J = 3.20 Hz, 1H), 8.58 (d, J = 3.20 |
| | Hz, 1H), 7.86 (d, J = 3.60 Hz, 1H), 7.57 (dd, J = 2.40, 8.80 Hz, 1H), 7.41 (dd, |
| | J = 0.80, 2.60 Hz, 1H), 7.34-7.31 (m, 2H), 4.33 (m, 2H), 4.09 (t, J = 4.80 Hz, |
| | 2H), 3.59-3.53 (m, 1H), 3.50-3.42 (m, 2H), 3.29-3.14 (m, 5H), 3.11-3.08 (m, |
| | 1H), 2.84-2.80 (m, 1H), 2.72 (t, J = 6.2 Hz, 3H), 2.56-2.55 (m, 2H), 2.50-2.50 |
| | (m, 1H), 2.34-2.32 (m, 2H), 2.09-2.05 (m, 1H), 1.91-1.80 (m, 2H), 1.59-1.58 |
| | (m, 4H) |
| 134 | 1H-NMR: (400 MHz, DMSO-d6): δ 8.80 (br s, 1H) 8.22 (br s, 1H), 7.57 (dd, J = |
| | 4.0, 8.0 Hz, 1H), 4.44-7.41 (m, 2H), 7.33 (d, J = 12 Hz, 2H), 4.31 (t, J = 4.0 Hz, |
| | 2H), 4.07-4.06 (m, 2H), 3.08 (s, 3H), 2.73 (s, 3H), 2.59-2.32 (m, 8H), 2.24-2.18 |
| | (m, 1H), 2.74-1.58 (m, 6H), 1.50-1.41 (m, 2H), 1.069 (s, 3H). |
| 135 | 1H-NMR: (400 MHz, DMSO-d6): δ 8.58 (s, 1H), 7.56 (dd, J = 2.80, 8.80 Hz, |
| | 1H), 7.42 (d, J = 2.40 Hz, 1H), 7.30-7.33 (m, 2H), 4.41-4.45 (m, 1H), 4.31 (d, |
| | J = 4.80 Hz, 2H), 4.06-4.08 (m, 2H), 2.76-2.84 (m, 2H), 2.70 (s, 3H), 2.60-2.68 |
| | (m, 3H), 2.38-2.45 (m, 4H), 2.15-2.22 (m, 1H), 1.85-1.96 (m, 3H), 1.69-1.71 |
| | (m, 2H), 1.70-1.56 (m, 2H), 1.05 (t, J = 6.80 Hz, 1H) |
| 136 | 1H-NMR: (400 MHz, , DMSO-d6): δ 8.65 (s, 1H), 8.41 (s, 1H), 7.57 (dd, J = |
| | 2.80, 8.80 Hz, 1H), 7.43 (d, J = 2.80 Hz, 1H), 7.33 (t, J = 9.20 Hz, 2H), 4.32- |
| | 4.45 (m, 3H), 4.06-4.08 (m, 2H), 2.81-2.84 (m, 2H), 2.66-2.70 (m, 5H), 2.60 |
| | (m, 2H), 2.38-2.40 (m, 4H), 1.88-1.93 (m, 3H), 1.77-1.06 (m, 2H), 1.69-1.65 |
| | (m, 2H) |
| 137 | 1H-NMR: (400 MHz, DMSO-d6): 8.81 (s, 1H), 7.59 (dd, J = 2.00, 8.80 Hz, 1H), |
| | 7.45 (d, J = 2.40 Hz, 2H), 7.34-7.32 (m, 1H), 4.33-4.30 (m, 2H), 4.04-4.08 (m, |
| | 2H), 2.74 (s, 3H), 2.50-2.51 (m, 3H), 2.44-2.40 (m, 5H), 2.22-2.15 (m, 1H), |
| | 2.09-1.80 (m, 3H), 1.55 (s, 3H), 1.58-1.56 (m, 3H), 1.24-1.22 (m, 2H). |
| 138 | 1H-NMR: (400 MHz, DMSO-d6): δ 8.28 (d, J = 4.0 Hz, 1H), 8.56 (br s, 1H), |
| | 8.21 (br s, 1H), 7.84 (d, J = 4 Hz, 1H), 7.56 (dd, J = 4.0, 8.0 Hz, 1H), 7.41 |
| | (d, J = 4.0 Hz, 1H), 7.31 (t, J = 4.0 Hz, 2H), 4.30-29 (m, 2H), 4.07-4.06 (m, |
| | 2H), 3.71 (s, 3H), 2.67-2.65 (m, 3H), 2.54-2.41 (m, 4H), 2.32-2.19 (m, 7H), |
| | 1.84 (d, J = 12.0 Hz, 1H), 1.60-1.50 (m, 4H). |
| 139 | 1H-NMR: (400 MHz, DMSO-d6): 8.81 (s, 1H), 7.58 (dd, J = 2.80, 8.80 Hz, 1H), |
| | 7.43-7.45 (m, 2H), 7.33 (d, J = 9.20 Hz, 1H), 4.35 (t, J = 5.60 Hz, 2H), 4.06 (t, |
| | J = 5.60 Hz, 2H), 2.73 (s, 3H), 2.67-2.49 (m, 8H), 2.23-2.19 (m, 1H), 1.87-1.84 |
| | (m, 1H), 1.65 (s, 3H), 1.64-1.52 (m, 5H), 1.28-1.18 (m, 2H). |
| 140 | 1H-NMR: (400 MHz, DMSO-d6): 8.64 (s, 1H), 7.57 (dd, J = 4.00, 10.00 Hz, |
| | 1H), 7.43 (d, J = 4.00 Hz, 1H), 7.36-7.30 (m, 2H), 4.31-4.30 (m, 2H), 4.08-4.07 |
| | (m, 2H), 2.71 (s, 3H), 2.66-2.49 (m, 8H), 2.34-2.32 (m, 1H), 1.90-1.87 (m, 1H), |
| | 1.63 (s, 3H), 1.54-1.48 (m, 5H), 1.28-1.16 (m, 2H) |
| 141 | 1H-NMR: (400 MHz, DMSO-d6): 8.43 (s, 1H), 7.55 (dd, J = 2.40, 9.00 Hz, 1H), |
| | 7.41 (d, J = 2.80 Hz, 1H), 7.27-7.31 (m, 2H), 4.31-4.30 (m, 2H), 4.08-4.07 (m, |
| | 2H), 2.68 (s, 3H), 2.63-2.46 (m, 8H), 2.21-2.17 (m, 1H), 1.89-1.84 (m, 1H), |
| | 1.62 (s, 3H), 1.51-1.58 (m, 5H), 1.24-1.15 (m, 2H). |
| 142 | 1H-NMR: (400 MHz, DMSO-d6): 10.22 (s. 1H), 8.68 (s, 1H), 7.57 (dd, J = 2.80, |
| | 9.00 Hz, 1H), 7.41 (d, J = 2.40 Hz, 1H), 7.31-7.36 (m, 2H), 4.32-4.30 (m, 2H), |
| | 4.07-4.08 (m, 2H), 3.91-3.99 (m, 2H), 3.32 (s, 3H), 3.24-3.32 (m, 1H), 2.73 (s, |
| | 3H), 2.67-2.73 (m, 4H), 2.32-2.51 (m, 6H), 1.91-2.00 (m, 3H), 1.64 (s, 3H), |
| | 1.59-1.49 (m, 3H). |
| 143 | 1H-NMR: (400 MHz, DMSO-d6): δ: 8.76 (brs, 1H), 8.19 (brs, 2H ), 8.11 (d, J = |
| | 5.2 Hz, 1H ), 7.58 (dd, J = 9.2 & 2.8 Hz, 1H), 7.40 (t, J = 2.8 Hz, 2H), 7.30 |
| | (d, J = 8.8 Hz, 1H), 6.98 (d, J = 5.2 Hz, 1H), 4.34 (brs, 2H), 4.08 (d, J = 4.0 Hz, |
| | 2H), 3.72 (s, 2H), 3.02-2.99 (m, 1H), 2.85-2.77 (m, 1H), 2.74 (s, 6H), 2.60- |
| | 2.55 (m, 2H), 2.50-2.48 (m, 1H), 2.35 (s, 3H), 2.02-1.93 (m, 1H), 1.80-1.70 |
| | (m, 1H), 1.58 (s, 3H). |
| 144 | 1H-NMR: (400 MHz, DMSO-d6): 10.22 (s, 1H), 8.68 (s, 1H), 7.57 (dd, J = 2.80, |
| | 9.00 Hz, 1H), 7.41 (d, J = 2.40 Hz, 1H), 7.31-7.36 (m, 2H), 4.39-4.37 (m, 1H), |
| | 4.33-4.31 (m, 2H), 4.1-4.09 (m, 2H), 3.42-3.50 (m, 4H), 3.42 (s, 3H), 3.20-3.29 |
| | (m, 5H), 2.76-2.86 (m, 3H), 2.73 (s, 3H), 2.52-2.67 (m, 2H), 2.06-2.33 (m, 3H), |
| | 1.81-1.91 (m, 2H), 1.64 (s, 3H), 1.59-1.58 (m, 1H). |
| 145 | 1H-NMR: (400 MHz, DMSO-d6): δ 10.33 (d, J = 12.0 Hz 1H), 8.60 (br s, 1H), |
| | 7.56 (dd, J = 4.0, 8.0 Hz, 1H), 7.41 (d, J = 4 Hz, 1H), 7.41 (m, 2H), 4.41 |
| | (s, 1H), 4.30-4.38 (m, 2H), 4.07-4.06 (m, 2H), 3.19-3.10 (m, 2H), 2.79-2.65 (m, |
| | 5H), 2.54-2.41 (m, 4H), 2.32-2.39 (m, 4H), 2.32-2.10 (m, 7H), 1.84 (d, J = 12.0 |
| | Hz, 1H), 1.60-1.50 (m, 4H). |
| 146 | 1H-NMR: (400 MHz, DMSO-d6): δ 7.55 (dd, J = 2.40, 9.00 Hz, 1H), 7.42 (d, |
| | J = 2.40 Hz, 1H), 7.32-7.29 (m, 2H), 4.31 (t, J = 4.80 Hz, 2H), 4.08 (t, J = 5.20 |
| | Hz, 2H), 3.32-3.18 (m, 2H), 3.05 (d, J = 9.20 Hz, 1H), 2.70 (s, 3H), 2.55-2.66 |
| | (m, 4H), 2.49-2.42 (m, 4H), 2.34-2.20 (m, 4H), 2.17-2.09 (m, 1H), 1.62 (s, 3H), |
| 147 | 1H-NMR: (400 MHz, DMSO-d6): δ 8.15 (s, 1H), 7.52 (dd, J = 2.80, 8.80 Hz, |
| | 1H), 7.38 (d, J = 2.40 Hz, 1H), 7.27 (d, J = 9.20 Hz, 1H), 7.12 (s, 1H), 4.28 (t, |
| | J = 4.80 Hz, 2H), 4.08 (t, J = 5.20 Hz, 2H), 3.22-3.17 (m, 1H), 3.14-3.12 (m, 2H), |
| | 2.87 (d, J = 9.20 Hz, 1H), 2.66 (s, 3H), 2.59-2.55 (m, 2H), 2.43-2.41 (m, 3H), |
| | 2.28-2.11 (m, 4H), 1.98-1.97 (m, 1H), 1.67-1.60 (m, 4H) |
| 148 | 1H-NMR: (400 MHz, DMSO-d6): δ 8.15 (s, 1H), 7.52 (dd, J = 2.80, 8.80 Hz, |
| | 1H), 7.38 (d, J = 2.40 Hz, 1H), 7.27 (d, J = 9.20 Hz, 1H), 7.12 (s, 1H), 4.28 (t, |
| | J = 4.80 Hz, 2H), 4.08 (t, J = 5.20 Hz, 2H), 3.22-3.17 (m, 1H), 3.14-3.12 (m, 2H), |
| | 2.87 (d, J = 9.20 Hz, 1H), 2.66 (s, 3H), 2.59-2.55 (m, 2H), 2.43-2.41 (m, 3H), |
| | 2.28-2.11 (m, 4H), 1.98-1.97 (m, 1H), 1.67-1.60 (m, 4H) |
| 149 | NMR 400 MHz, DMSO-d6): δ 8.70 (s, 1H), 7.57 (dd, J = 2.40, 9.00 Hz, 1H), |
| | 7.44 (d, J = 2.40 Hz, 1H), 7.38 (d, J = Hz, 1H), 7.32 (s, 1H), 4.31 (t, J = 4.80 |
| | Hz, 2H), 4.09 (t, J = 4.00 Hz, 2H), 3.32-3.29 (m, 1H), 3.21-3.12 (m, 1H), 2.88 |
| | (d, J = 9.20 Hz, 1H), 2.73 (s, 3H), 2.60-2.59 (m, 3H), 2.43-2.41 (m, 3H), 2.22- |
| | 2.09 (m, 4H), 2.02-1.99 (m, 1H), 1.68-1.59 (m, 4H) |
| 150 | 1H-NMR: (400 MHz, DMSO-d6): δ 10.33 (d, J = 12.0 Hz 1H), 8.60 (br s, 1H), |
| | 7.56 (dd, J = 4.0, 8.0 Hz, 1H), 7.41 (d, J = 4 Hz, 1H), 7.41 (m, 2H), 4.41 |
| | (s, 1H), 4.30-4.38 (m, 2H), 4.07-4.06 (m, 2H), 3.19-3.10 (m, 2H), 2.79-2.65 (m, |
| | 3H), 2.54-2.41 (m, 4H), 2.32-2.39 (m, 4H), 2.32-2.10 (m, 7H), 1.84 (d, J = 12.0 |
| | Hz, 1H), 1.60-1.50 (m, 4H). |
| 151 | 1H-NMR: (400 MHz, DMSO-d6): δ 8.80 (s, 1H), 8.25 (s, 2H), 7.57 (dd, J = |
| | 2.80, 8.80 Hz, 1H), 7.44 (d, J = 2.80 Hz, 1H), 7.39 (s, 1H), 7.33 (d, J = 8.80 Hz, |
| | 1H), 4.37-4.18 (m, 2H), 4.11-4.01 (m, 2H), 3.22 (s, 3H), 3.19-3.01 (m, 1H), |
| | 2.80-2.66 (m, 6H), 2.63-2.59 (m, 1H), 2.33-2.20 (m, 3H), 1.96 (t, J = 12.0 Hz, |
| | 1H), 1.82 (m, 2H), 1.64 (s, 3H), 1.41-1.31 (m, 4H), 0.77-0.74 (m, 1H). |
| 152 | 1H-NMR: (400 MHz, DMSO-d6): δ 8.77 (s, 1H), 7.58 (dd, J = 2.40, 8.80 Hz, |
| | 1H), 7.44 (d, J = 2.80 Hz, 1H), 7.41 (s, 1H), 7.33 (d, J = 9.20 Hz, 1H), 4.32 (t, |
| | J = 4.80 Hz, 2H), 4.07 (t, J = 4.80 Hz, 2H), 4.08-4.06 (m, 1H), 3.31 (s, 3H), 2.73- |
| | 2.66 (m, 5H), 2.44-2.40 (m, 5H), 2.34-2.31 (m, 1H), 2.21-2.03 (m, 3H), 1.88- |
| | 1.85 (m, 1H), 1.64-1.54 (m, 8H) |
| 153 | 1H-NMR: (400 MHz, DMSO-d6): δ: 8.74 (s, 1H), 8.32 (s, 1H), 7.58-7.56 (dd, |
| | J = 2.8 Hz, 9.2 Hz, 1H), 7.43 (d, J = 2.4 Hz, 1H), 7.39 (s, 1H), 7.32 (d, J = 8.8 |
| | Hz, 1H) , 4.32 (t, J = 4.4 Hz, 2H), 4.07 (t , J-4.8 Hz, 2H), 3.33-2.63 (m, 6H), |
| | 2.58-2.51 (m, 2H), 2.59-2.46 (m, 1H), 2.44-2.33 (m, 4H), 2.04-1.97 (dd, J = 7.6 |
| | Hz, 16.8 Hz, 1H), 1.86-1.83 (m, 1H), 1.59-1.55 (m, 4H). |
| 154 | 1H-NMR: (400 MHz, DMSO-d6): δ 8.79 (s, 1H), 8.05 (d, J = 5.20 Hz, 1H), |
| | 7.58 (dd, J = 2.40, 9.00 Hz, 1H), 7.45-7.42 (m, 2H), 7.32 (d, J = 8.80 Hz, 1H), |
| | 6.99-6.97 (m, 1H), 6.80 (s, 1H), 4.32 (t, J = 4.80 Hz, 2H), 4.06 (t, J = 4.80 Hz, |
| | 2H), 3.81 (s, 3H), 3.80-3.76 (m, 2H), 3.21-3.17 (m, 1H), 2.73 (s, 3H), 2.72-2.71 |
| | (m, 1H), 2.59-2.45 (m, 3H), 2.10-2.04 (m, 1H), 1.87-1.83 (m, 1H), 1.58 (s, 3H), |
| | 1.57-1.53 (m, 1H). |
| 155 | 1H NMR (400 MHz, DMSO-d6): δ 8.71 (s, 1H), 7.58 (dd, J = 2.80, 9.00 Hz, |
| | 1H), 7.43 (dd, J = 2.80, Hz, 2H), 7.36 (s, 1H), 7.33 (dd, J = 9.20, Hz, 1H), |
| | 4.37-4.33 (m, 2H), 4.12-4.08 (m, 2H), 3.53 (s, 2H), 3.01-2.98 (m, 1H), 2.80- |
| | 2.70 (m, 1H), 2.67-2.64 (m, 6H), 2.59-2.52 (m, 2H), 2.39-2.32 (m, 2H), 1.93- |
| | 1.91 (m, 1H), 1.84-1.79 (m, 1H), 1.55 (s, 3H) |
| 156 | 1H-NMR: (400 MHz, DMSO-d6): 8.77 (s, 1H), 7.58 (dd, J = 2.40, 9.00 Hz, 1H), |
| | 7.42 (dd, J = 11.20 Hz, 1H), 7.34-7.31 (m, 1H), 4.37-4.32 (m, 2H), 4.07-4.05 |
| | (m, 2H), 2.73-2.98 (m, 2H), 2.51 (s, 3H), 2.50-2.50 (m, 2H), 2.21-2.23 (m, 3H), |
| | 2.12-2.20 (m, 6H), 1.91-1.80 (m, 2H), 1.65 (s, 3H), 1.62-1.36 (m, 3H), -1.43- |
| | 1.24 (m, 1H), 1.15-1.18 (m, 1H), 1.14-1.10 (m, 1H) |
| 157 | 1H-NMR: (400 MHz, DMSO-d6): δ 8.89 (s, 1H), 8.77 (s, 1H), 8.52 (s, 1H), |
| | 7.58 (dd, J = 2.80, 8.80 Hz, 1H), 7.44-7.42 (m, 2H), 7.33 (d, J = 9.20 Hz, 1H), |
| | 4.34-4.30 (m, 2H), 4.08-4.07 (m, 2H), 3.85-3.76 (m, 2H), 3.16-2.86 (m, 5H), |
| | 2.73 (s, 3H), 2.67-2.66 (m, 3H), 2.49-2.34 (m, 1H), 2.00-1.98 (m, 1H), 1.91- |
| | 1.73 (m, 1H), 1.58 (s, 3H). |
| 158 | 1H-NMR: (400 MHz, DMSO-d6): δ 8.69 (s, 1H), 8.15 (d, J = 4.80 Hz, 1H), |
| | 7.57 (dd, J = 2.40, 9.00 Hz, 1H), 7.31-7.44 (m, 3H), 6.67 (d, J = 4.80 Hz, 1H), |
| | 6.49 (s, 2H), 4.30-4.34 (m, 2H), 4.07-4.08 (m, 2H), 3.55-3.43 (m, 2H), 2.72- |
| | 2.71 (m, 1H), 2.69 (s, 3H), 2.51-2.49 (m, 3H), 2.27 (s, 3H), 2.26-2.23 (m, 1H), |
| | 1.94-1.91 (m, 1H), 1.58 (s, 3H), 1.59-1.24 (m, 1H). |
| 159 | 1H-NMR: (400 MHz, DMSO-d6): δ 8.76 (s, 1H), 7.58 (dd, J = 2.80, 8.80 Hz, |
| | 1H), 7.43 (dd, J = 2.40, Hz, 2H), 7.34-7.30 (m, 2H), 4.37-4.33 (m, 2H), 4.11- |
| | 4.07 (m, 2H), 3.62 (s, 2H), 3.25 (d, 1H), 2.97-2.93 (m, 7H), 2.57-2.56 (m, 1H), |
| | 2.51-2.50 (m, 3H), 1.94-1.91 (m, 1H), 1.78-1.77 (m, 1H), 1.56 (s, 3H) |
| 160 | 1H-NMR: (400 MHz, DMSO-d6): δ: 8.61 ( s, 1H), 7.588 (dd, J = 11.2 Hz 1H), |
| | 7.44 (m, 4H), 7.32 (m, 2H), 4.33 (m, 2H), 4.07 (m, 2H), 3.84 (m, 2H), 2.95 (m, |
| | 2H), 2.77 (m, 6H), 2.56 (s, 2H), 2.37 (m, 2H), 1.98 (d, J = 9.8 Hz 1H), 1.71 (s, |
| | 1H), 1.581 (s, 3H), |
| 161 | 1H-NMR: (400 MHz, DMSO-d6): δ 8.84 (s, 1H), 7.57 (dd, J = 2.80, 8.80 Hz, |
| | 1H), 7.42 (t, J = 8.00 Hz, 2H), 7.33 (d, J = 9.20 Hz, 1H), 4.34-4.33 (m, 2H), |
| | 4.08-4.05 (m, 2H), 3.03 (d, J = 6.80 Hz, 2H), 2.97 (d, J = 6.40 Hz, 2H), 2.73 (s, |
| | 1H), 2.51-2.50 (m, 4H), 2.39-2.29 (m, 1H), 2.01-1.96 (m, 1H), 1.83-1.80 (m, |
| | 5H), 1.73-1.66 (m, 5H), 1.58 (s, 3H), 1.48-1.47 (m, 1H). |
| 162 | 1H-NMR: 400 MHz, DMSO-d6): δ 8.90 (s, 1H), 7.58 (dd, J = 2.80, 9.00 Hz, |
| | 1H), 7.44 (d, J = 3.20 Hz, 2H), 7.33 (d, J = 8.80 Hz, 1H), 5.10 (d, J = 16.40 Hz, |
| | 1H), 4.36-4.33 (m, 2H), 4.07-4.03 (m, 2H), 3.09 (d, J = 6.80 Hz, 1H), 3.02-2.97 |
| | (m, 2H), 2.91 (d, J = 6.80 Hz, 1H), 2.73 (s, 3H), 2.45-2.40 (m, 2H), 2.32-2.29 |
| | (m, 1H), 2.28-2.24 (m, 1H), 2.18-2.11 (m, 4H), 1.99 (dd, J = 6.00, 17.20 Hz, |
| | 1H), 1.81-1.72 (m, 2H), 1.69-1.66 (m, 1H), 1.58 (s, 3H), 1.51-1.48 (m, 1H). |
| 163 | 1H-NMR: (400 MHz, DMSO-d6): δ 8.72 (s, 1H), 7.57 (dd, J = 2.80, 9.00 Hz, |
| | 1H), 7.43 (d, J = 2.80 Hz, 1H), 7.39 (s, 1H), 7.32 (d, J = 8.80 Hz, 1H), 4.32- |
| | 4.31 (m, 2H), 4.08-4.06 (m, 2H), 3.01 (d, J = 11.60 Hz, 1H), 2.97 (d, J = 11.60 |
| | Hz, 1H), 2.72 (s, 3H), 2.82-2.62 (m, 1H), 2.49-2.47 (m, 3H), 2.26-2.26 (m, |
| | 4H), 1.90-1.75 (m, 3H), 1.63-1.59 (m, 4H), 1.51-1.35(m, 2H). |
| 164 | 1H-NMR: (400 MHz, DMSO-d6): δ 8.87 (s, 1H), 8.78 (s, 1H), 7.58 (dd, J = |
| | 2.80, 8.80 Hz, 1H), 7.44 (t, J = 1.20 Hz, 2H), 7.33 (d, J = 9.20 Hz, 1H), 4.34- |
| | 4.32 (m, 2H), 4.18-4.08 (m, 2H), 3.87 (s, 2H), 3.05-3.03 (m, 1H), 2.87-2.83 (m, |
| | 4H), 2.73 (s, 3H), 2.42-2.35 (m, 4H), 1.95-1.98 (m, 1H), 1.81-1.74 (m, 1H), |
| | 1.56 (s, 3H). |
| 165 | 1H-NMR (400 MHz, DMSO-d6): δ 13.51 (bs, 1H), 8.79 (s, 1H), 8.29 (dd, J = |
| | 1.20, Hz, 1H), 7.58 (dd, J = 2.80, 8.80 Hz, 1H), 7.51 (d, J = 6.80 Hz, 1H), 7.43 |
| | (d, J = 1.60 Hz, 2H), 7.33 (d, J = 9.20 Hz, 1H), 7.15-7.12 (m, 1H), 4.35 (t, J = |
| | 3.20 Hz, 2H), 4.07 (t, J = 3.20 Hz, 2H), 3.78 (s, 2H), 2.99 (m, 1H), 2.96-2.81( |
| | m, 7H), 2.70 (s, 2H ) , 2.4 (s 2H ), 2.10-1.9 (m 1H), 1.80-1.78 (m 1H), 1.6 (s, 3H). |
| 166 | 1H-NMR: (400 MHz, DMSO-d6): δ 8.68 (s, 1H), 7.57 (dd, J = 2.80, 9.00 Hz, |
| | 1H), 7.43 (d, J = 2.80 Hz, 1H), 7.36 (s, 1H), 7.32 (d, J = 8.80 Hz, 1H), 4.31 (t, |
| | J = 4.80 Hz, 2H), 4.07 (t, J = Hz, 2H), 2.72 (s, 3H), 2.62-2.66 (m, 5H), 2.33-2.50 |
| | (m, 4H), 2.17-2.25 (m, 2H), 2.17-2.25 (m, 1H), 1.93-2.03 (m, 2H), 1.78-1.84 |
| | (m, 1H), 1.60 (s, 3H), 1.44-1.49 (m, 1H), 1.24 (s, 1H). |
| 167 | 1H-NMR: (400 MHz, DMSO-d6): δ 8.57 (s, 1H), 7.56 (dd, J = 2.80, 8.80 Hz, |
| | 1H), 7.42 (d, J = 2.40 Hz, 1H), 7.31 (d, J = 8.80 Hz, 2H), 4.31 (t, J = 4.80 Hz, |
| | 2H), 4.08 (t, J = 4.80 Hz, 2H), 2.73 (s, 3H), 2.64-2.71 (m, 3H), 2.54-2.59 (m, |
| | 3H), 2.41-2.49 (m, 2H), 2.23-2.33 (m, 2H), 2.02-2.10 (m, 1H), 1.90-2.00 (m, |
| | 2H), 1.83-1.87 (m, 1H), 1.58 (s, 3H), 1.52-1.55 (m, 2H), 1.24 (s, 1H). |
| 168 | 1H-NMR: (400 MHz, DMSO-d6): δ 8.61 (s, 1H), 7.56 (dd, J = 2.40, 8.80 Hz, |
| | 1H), 7.42 (d, J = 2.80 Hz, 1H), 7.33 (d, J = 6.40 Hz, 1H), 7.30 (s, 1H), 4.32 (t, |
| | J = 4.8 Hz, 2H), 4.08 (t, J = 4.8 Hz, 2H), 2.73 (s, 3H), 2.67-2.71 (m, 3H), 2.55- |
| | 2.59 (m, 3H), 2.38-2.50 (m, 2H), 2.17-2.25 (m, 2H), 2.02-2.08 (m, 2H), 1.90- |
| | 2.01 (m, 1H), 1.75-1.83 (m, 1H), 1.58 (s, 3H), 1.58 (m, 2H), 1.24 (s, 1H). |
| 169 | 1H-NMR: (400 MHz, DMSO-d6): δ 8.39 (s, 1H), 7.54 (dd, J = 2.40, 8.80 Hz, |
| | 1H), 7.40 (d, J = 2.40 Hz, 1H), 7.29 (d, J = 9.20 Hz, 1H), 7.22 (s, 1H), 4.30 (t, |
| | J = 4.0 Hz, 2H), 4.08 (t, J = 4.0 Hz, 2H), 3.22 (m, 1H), 2.85-2.87 (m, 1H), 2.68 |
| | (s, 3H), 2.60-2.66 (m, 3H), 2.41-2.50 (m, 3H), 2.20-2.33 (m, 2H), 1.88-1.96 (m, |
| | 4H), 1.80-1.83 (m, 1H), 1.57 (s, 3H), 1.43-1.48 (m, 1H), 1.23 (s, 1H). |
| 170 | 1H-NMR (400 MHz, DMSO-d6): δ 8.2 (brs, 1H), 7.53 (dd, J = 2.40, 8.80 Hz, |
| | 1H), 7.38 (d, J = 2.40 Hz, 1H), 7.29 (d, J = 8.80 Hz, 1H), 7.18 (brs, 1H), 4.27 |
| | (d, J = 4.80 Hz, 2H), 4.08 (s, 2H), 2.69-2.60 (m, 6H), 2.45-2.12 (m, 5H), 1.98- |
| | 1.70 (m, 6H), 1.65 (s, 3H), 1.60- 1.51 (m, 7H). |
| 171 | 1H-NMR: (400 MHz, DMSO-d6): δ: 13.11 (br. s, exchange with D20, 1H), |
| | 8.73 (s, 1H), 7.59 (dd, J = 2.8 Hz, 9.0 Hz, 1H), 7.46-7.41 (m, 3H), 7.33 (d, J = |
| | 9.2 Hz, 1H), 4.36 (t, J = 4.8 Hz, 2H), 4.33 (t, J = 4.8 Hz, 2H), 3.85-3.76 (m, |
| | 2H), 2.72 (s, 4H), 2.58 (s, 3H), 2.51 (m, 1H), 2.46 (s, 2H), 2.27 (m, 4H), 1.90 |
| | (s, 1H), 1.69 (m, 4H). |
| 172 | 1H-NMR: (400 MHz, DMSO-d6): δ: 8.71 (s, 1H), 8.32 (s, 1H), 7.59-7.56 (dd, |
| | J = 2.4 Hz, 8.8 Hz, 1H), 7.43 (d, J = 2.4 Hz, 1H), 7.37 (s, 1H), 7.32(d, J = 12 Hz, |
| | 1H), 4.32 (t, J = 2.4 Hz, 2H), 4.07 (t, J = 4.8Hz, 2H), 3.04-2.88 (m, 4H), 2.73 (s, |
| | 3H), 2.46-2.42 (m, 6H), 2.37-2.32 (m, 1H), 1.93-1.91 (m, 1H), 1.57 (m, 4H). |
| 173 | 1H-NMR: (400 MHz, DMSO-d6): 8.78 (s, 1H), 7.58 (dd, J = 2.4, 8.8 Hz, 1H), |
| | 7.45-7.42 (m, 2H), 7.34-7.29 (m, 2H), 6.58 (d, J = 7.2 Hz, 1H), 6.27 (d, J = 8.4 |
| | Hz, 1H), 5.80 (s, 2H), 4.34-4.31 (m, 2H), 4.07-4.03 (m, 2H), 3.56-3.45 (m, 2H), |
| | 2.69 (s, 3H), 2.73-2.70 (m, 1H), 2.51-2.50 (m, 3H), 2.27 (s, 3H), 2.26-2.23 (m, |
| | 1H), 1.94-1.91 (m, 1H), 1.58 (s, 3H), 1.59-1.24 (m, 1H). |
| 174 | 1H NMR (400 MHz, DMSO-d6): δ 8.32 (s, 1H), 7.57 (dd, J = 11.60, Hz, 1H), |
| | 7.39-7.44 (m, 2H), 7.33 (d, J = 9.20 Hz, 1H), 4.32 (m, 2H), 4.07 (m, 2H), 2.73 |
| | (s, 3H), 2.58-2.67 (m, 4H), 2.50-2.51 (m, 3H), 2.18-2.25 (m, 1H), 1.88 (d, J = |
| | 12.00 Hz, 1H), 1.53-1.59 (m, 9H), 1.21-1.25 (m, 2H), |
| 175 | 1H-NMR: (400 MHz, DMSO-d6): δ 8.81 (s, 1H), 7.58 (dd, J = 2.80, 9.00 Hz, |
| | 1H), 7.47-7.43 (m, 2H), 7.33 (d, J = 8.80 Hz, 1H), 4.34 (t, J = 4.80 Hz, 2H), |
| | 4.07 (t, J = 4.40 Hz, 2H), 2.74 (s, 3H), 2.67-2.63 (m, 3H), 2.55-2.50 (m, 3H), |
| | 2.38-2.33 (m, 2H), 2.63-2.22 (m, 5H), 1.95-1.93 (m, 3H), 1.59-1.57 (m, 4H) |
| 176 | 1H-NMR: (400 MHz, DMSO-d6): δ: 8.71 (s , 1H), 7.59-7.56 (dd, J = 2.4 Hz, |
| | J = 8.8 Hz, 1H), 7.43 (d, J = 2.4 Hz, 1H), 7.37 (s, 1H), 7.32 (d, J = 12 Hz, 1H), |
| | 4.30-4.28 (m, 2H), 4.08-4.07 (m, 2H), 2.33-2.69 (m, 4H), 2.6 (d, J = 6.4Hz, |
| | 1H), 2.52-2.41(m, 4H), 2.25 (d, J = 10 Hz, 2H), 2.19-2.12 (m, 3H), 1.86 (d, J = |
| | 11.2 Hz, 1H), 1.60-1.41 (m, 9H), 1.32 (d, J = 10.4 Hz, 1H). |
| 177 | 1H-NMR: (400 MHz, DMSO-d6): δ: 13.3 (bs, 1H), 8.76 (s, 1H), 8.17 (s, 1H), |
| | 7.59-7.56 (dd, J = 8.8 Hz, J = 8.8, 1H), 7.38 (d, J = 2.4 Hz, 1H), 7.41 (s, 1H), |
| | 7.31 (d, J = 8.8 Hz, 1H), 6.95 (s, 1H), 4.33 (m, 2H), 4.08 (t, J = 4.8 Hz, 2H), 3.77- |
| | 3.71 (m, 2H), 2.93-2.91 (m, 1H), 2.76-2.66 (m, 8H), 2.56 (t, J = 4.4 Hz, 2H), |
| | 2.33-2.32 (m, 4 H), 1.69 (d, J = 11.6 Hz, 1H), 1.76-1.69 (m, 1H), 1.58 (s, 3H). |
| 178 | 1H-NMR: (400 MHz, DMSO-d6): δ: 8.27 (d, J = 14 Hz, 1H), 7.56 (dd, J = 8.8, |
| | 9.2 Hz, 1H), 7.40 (d, J = 2.4 Hz, 1H), 7.29 (d, J = 8.8 Hz, 1H), 7.25 (s, 1H), |
| | 4.43-4.41 (m, 1H), 4.30-4.28 (m, 2H), 4.08-4.07 (m, 2H), 2.83-2.74 (m, |
| | 2H), 2.67 (s, 3H), 2.67-2.37 (m, 6H), 2.21-2.15 (m, 1H), 1.92-1.85 (m, 3H), |
| | 1.69-1.51 (m, 6H). |
| 179 | 1H-NMR: (400 MHz, DMSO-d6): δ: 8.27 (s, 1H), 7.59-7.56 (dd, J = 8.8 Hz, 8.8 |
| | Hz, 1H), 7.44 (d, J = 2.4 Hz, 1H), 7.42 (s, 1H), 7.31 (d, J = 8.8 Hz, 1H), 4.42 (m, |
| | 2H), 4.06 (m, 2H), 3.41-3.28 (m, 2H), 2.90-2.80 (m, 2H), 2.73 (s, 3H), 2.63 (m, |
| | 1H), 2.46-2.18 (m, 6H), 1.90-1.87 (m, 3H), 1.63-1.57 (m, 4H), 1.49-1.45 (m, |
| | 2H), 0.44-0.41 (m, 4H). |
| 180 | 1H-NMR: (400 MHz, DMSO-d6): δ 8.30 (d, J = 3.6 Hz, 1H), 8.28 (s, 1H), 7.54 |
| | (dd, J = 2.80, 8.80 Hz, 1H), 7.47 (d, J = 7.60 Hz, 1H), 7.39 (d, J = 2.40 Hz, 1H), |
| | 7.29 (d, J = 9.20 Hz, 1H), 7.15-7.11 (m, 2H), 4.30-4.29 (m, 2H), 4.09-4.04 (m, |
| | 2H), 3.81-3.73 (m, 2H), 2.99-2.80 (m, 6H), 2.64-2.59 (m, 3H), 2.56-2.52 (m, |
| | 2H), 2.37-2.31 (m, 1H), 2.01-1.98 (m, 1H), 1.73-1.64 (m, 4H). |
| 181 | 1H-NMR: (400 MHz, DMSO-d6): δ 8.73 (s, 1H), 7.58 (dd, J = 2.80, 8.80 Hz, |
| | 1H), 7.44-7.41 (m, 2H), 7.33 (d, J = 8.80 Hz, 1H), 7.23 (d, J = 5.20 Hz, 1H), |
| | 6.80 (d, J = 5.20 Hz, 1H), 4.35-4.32 (m, 2H), 4.08-4.06 (m, 2H), 3.65 (s, 2H), |
| | 3.00-2.82 (m, 1H), 2.82-2.73 (m, 7H), 2.52-2.51 (m, 2H), 2.45-2.40 (m, 2H), |
| | 1.97-1.91 (m, 1H), 1.77-1.75 (m, 1H), 1.57 (s, 3H). |
| 182 | 1H-NMR: (400 MHz, DMSO-d6): δ: 13.63 (bs, 1H), 8.78 (s, 1H), 7.58 (dd, 8.8, |
| | 2.8 Hz, 1H), 7.44 (d, J = 2.8 Hz, 1H), 7.43 (s, 1H), 7.33 (d, J = 9.2 Hz, 1H), |
| | 7.11 (s, 1H), 4.33-4.29 (m, 2H), 4.09-4.07 (m, 2H), 3.91-3.89 (m, 2H), |
| | 2.85-2.77 (m, 1H), 2.73 (s, 3H), 2.55-2.51 (m, 3H), 2.34 (s, 3H), 2.31(s, 3H), |
| | 2.23-2.20 (m, 1H), 1.94-1.90 (m, 1H), 1.70-1.61 (m, 1H), 1.58 (s, 3H). |
| 183 | 1H-NMR: (400 MHz, DMSO-d6): δ 8.66 (s, 1H), 7.57 (dd, J = 2.40, 8.80 Hz, |
| | 1H), 7.44 (d, J = 2.80 Hz, 1H), 7.37 (s, 1H), 7.32 (d, J = 8.80 Hz, 1H), 4.35-4.28 |
| | (m, 2H), 4.08-4.07 (m, 2H), 3.13-3.05 (m, 1H), 2.89-2.80 (m, 1H), 2.72 (s, 3H), |
| | 2.46 (m, 2H), 2.40-2.39 (m, 1H), 2.23-2.18 (m, 1H), 2.157 (s, 3H), 1.80-1.74 |
| | (m, 3H), 1.72-1.62 (m, 4H), 1.603 (s, 3H), 1.45-1.37 (m, 1H), 1.19-1.23 (m, |
| | 3H), 1.156 (d, J = Hz, 1H). |
| 184 | 1H-NMR: (400 MHz, DMSO-d6): δ 8.58 (s, 1H), 7.56 (dd, J-2.80, 8.80 Hz, |
| | 1H), 7.43 (d, J = 2.40 Hz, 1H), 7.33 (d, J = 2.00 Hz, 1H), 7.30 (s, 1H), 4.32-4.30 |
| | (m, 2H), 4.11-4.05 (m, 2H), 3.08 (t, J = 8.00 Hz, 1H), 2.85-2.75 (m, 1H), 2.71 |
| | (s, 3H), 2.49-2.47 (m, 2H), 2.40-2.33 (m, 1H), 2.22-2.18 (m, 1H), 2.16 (s, 3H), |
| | 1.81-1.60 (m, 7H), 1.59 (s, 3H), 1.46-1.41 (m, 1H), 1.23 (s, 1H), 1.18 (s, 3H), |
| | 1.14-1.15 (m, 1H). |
| 185 | 1H-NMR: (400 MHz, DMSO-d6): δ: 13.11 (br. s, exchange with D20, 1H), |
| | 8.73 (s, 1H), 7.59 (dd, J = 2.8 Hz, 8.8 Hz, 1H), 7.46-7.41 (m, 3H), 7.32 (d, J = |
| | 9.2 Hz, 1H), 4.33 (t, J = 4.8 Hz, 2H), 4.06 (t, J = 4.8 Hz, 2H), 3.85-3.76 (m, |
| | 2H), 2.72 (s, 4H), 2.58 (s, 3H), 2.51 (m, 1H), 2.46 (s, 2H), 2.27-2.21 (m, 4H), |
| | 1.90 (s, 1H), 1.69-1.58 (m, 4H). |
| 186 | 1H-NMR: (400 MHz, DMSO-d6): δ 8.30 (s, 1H), 7.58 (dd, J = 4.00, 8.00 Hz, |
| | 1H), 7.45-7.42 (m, 2H), 7.34-7.29 (m, 2H), 6.10 (d, J = 7.2 Hz, 1H), 4.33-4.31 |
| | (m, 2H), 4.08-4.06 (m, 2H), 3.67-3.57 (m, 2H), 2.69 (s, 3H), 2.66-2.62 (m, 2H), |
| | 2.51-2.49 (m, 2H), 2.31-2.28 (m, 1H), 2.27 (s, 3H), 1.93-1.83 (m, 1H), 1.62 (s, |
| | 3H), 1.58-1.55 (m, 1H), 1.21-1.15 (m, 1H). |
| 187 | 1H-NMR: (400 MHz, DMSO-d6): δ: 9.06 (s, 1H), 8.74-8.66 (m, 3H), 7.59 |
| | (dd, 8.8, 1.6 Hz, 1H), 7.44 (d, J = 2.8 Hz, 1H), 7.39 (s, 1H), 7.33 (d, J = 8.8 Hz, |
| | 1H), 4.33-4.31 (m, 2H), 4.09-4.07 (m, 2H), 3.74-3.62 (m, 2H), 2.78-2.66 |
| | (m, 5H), 2.59-2.51 (m, 3H), 2.28-2.18 (m, 5H), 1.97-1.94 (m, 1H), 1.73- |
| | 1.61 (m, 4H). |
| 188 | 1H-NMR: (400 MHz, DMSO-d6): δ 9.19 (s, 1H), 9.13 (dd, J = 1.20, 5.20 Hz, |
| | 1H), 8.74 (s, 1H), 7.62 (dd, J = 2.00, 5.20 Hz, 1H), 7.58 (dd, J = 2.40, 8.80 Hz, |
| | 1H), 7.44 (d, J = 2.40 Hz, 1H), 7.41 (s, 1H), 7.33 (d, J = 8.80 Hz, 1H), 4.33 (d, |
| | J = 3.20 Hz, 2H), 4.08-4.07 (m, 2H), 3.75-3.69 (m, 2H), 2.77-2.73 (m, 1H), 2.67 |
| | (s, 3H), 2.57-2.56 (m, 2H), 2.45-2.44 (m, 1H), 2.33-2.25 (m, 1H), 2.21 (s, 3H), |
| | 1.97-1.94 (m, 1H), 1.69-1.63 (m, 1H), 1.60 (s, 3H). |
| 189 | 1H-NMR: (400 MHz, DMSO-d6): δ: 8.61 (s, 1H), 8.42 (s, 1H), 7.57 (dd, J = |
| | 8.8, 2.8 Hz, 1H), 7.43 (d, J = 2.8 Hz, 1H), 7.35 (s, 1H), 7.31 (d, J = 9.2 Hz, 1H), |
| | 4.35-4.28 (m, 2H), 4.10-4.02 (m, 2H), 3.20-2.95 (m, 3H), 2.73 (s, 3H), 2.59- |
| | 2.56 (m, 1H), 2.55-2.42 (m, 6H), 2.22-2.09 (m, 6H), 1.84-1.50 (m, 7H). |
| 190 | 1H-NMR: (400 MHz, DMSO-d6): δ 8.77 (s, 1H), 8.17 (s, 1H), 7.58 (dd, J = |
| | 11.60, Hz, 1H), 7.42-7.45 (m, 2H), 7.33 (d, J = 8.80 Hz, 1H), 6.89 (s, 2H), |
| | 4.31-4.35 (m, 2H), 4.07 (m, 2H), 3.62-3.71 (m, 2H), 2.73 (s, 3H), 2.49-2.51 (m, |
| | 4H), 2.21-2.28 (m, 4H), 1.90-1.93 (m, 1H), 1.58-1.63 (m, 4H), |
| 191 | 1H-NMR: (400 MHz, DMSO-d6): δ 8.27 (s, 1H), 7.57 (dd, J = 2.40, 9.0 Hz, |
| | 1H), 7.47-7.44 (m, 2H), 7.39 (s, 1H), 7.34-7.30 (m, 2H), 7.05 (dd, J = 0.80, 5.0 |
| | Hz, 1H), 4.34-4.31 (m, 2H), 4.08-4.07 (m, 2H), 3.64 (d, J = 10.40 Hz, 2H), |
| | 2.73-2.71 (m, 4H), 2.50-2.47 (m, 3H), 2.29-2.18 (m, 4H), 1.92 (d, J = 14.0 Hz, |
| | 1H), 1.68-1.59 (m, 4H). |
| 192 | 1H-NMR: (400 MHz, DMSO-d6): δ 8.83 (s, 1H), 7.58 (dd, J = 2.80 Hz, 1H), |
| | 7.44 (t, J = 3.20 Hz, 2H), 7.33 (d, J = 9.20 Hz, 1H), 4.34-4.31 (m, 2H), 4.08- |
| | 4.07 (m, 2H), 2.74-2.65 (m, 9H), 2.50-2.49 (m, 2H), 2.33-2.27 (m, 1H), 1.93- |
| | 1.90 (m, 1H), 1.68-1.58 (m, 4H), 1.50-1.38 (m, 4H), 0.27 (s, 4H). |
| 193 | 1H NMR (400 MHz, DMSO-d6): δ 8.69 (s, 1H), 7.57 (dd, J = 2.40, 8.80 Hz, |
| | 1H), 7.44 (d, J = 2.40 Hz, 1H), 7.38 (s, 1H), 7.32 (d, J = 9.20 Hz, 1H), 4.68 (s, |
| | 1H), 4.32 (t, J = 6.00 Hz, 2H), 4.06 (t, J = 5.20 Hz, 2H), 3.32 (m, 1H), 2.95 (s, |
| | 3H), 2.68-2.67 (m, 1H), 2.47-2.45 (m, 2H), 2.35-2.31 (m, 1H), 2.18-2.15 (m, |
| | 3H), 2.02-1.98 (m, 3H), 1.80-1.72 (m, 3H), 1.60 (s, 3H), 1.55 (m, 2H), 1.20 (s, 3H), |
| 194 | 1H-NMR: (400 MHz, DMSO-d6): δ 8.82 (s, 1H), 7.58 (dd, J = 2.40, 9.00 Hz, |
| | 1H), 7.45 (s, 2H), 7.33 (d, J = 8.80 Hz, 1H), 4.34 (t, J = 4.80 Hz, 2H), 4.07 (t, |
| | 2H), 2.94 (m, 4H), 2.74 (s, 3H), 2.51-2.50 (m, 5H), 2.51-2.50 (m, 3H), 2.33- |
| | 2.32 (m, 1H), 1.99 (s, 3H), 1.68-1.67 (m, 1H), 1.57 (s, 3H) |
| 195 | 1H-NMR: (400 MHz, DMSO-d6): δ 8.82 (s, 1H), 7.59 (dd, J = 2.80, 9.00 Hz, |
| | 1H), 7.45 (d, J = 3.60 Hz, 2H), 7.33 (dd, J = 9.20, Hz, 1H), 4.36-4.08 (m, 8H), |
| | 3.45-3.40 (m, 1H), 2.74 (s, 4H), 2.60-2.58 (m, 2H), 2.50-2.39 (m, 5H), 2.15- |
| | 2.01 (m, 2H), 1.57 (s, 4H). |
| 196 | 1H-NMR: (400 MHz, DMSO-d6): δ: 13.7 (Br, s, 1H) 8.78 (s, 1H), 8.51 (s, 1H), |
| | 8.44 (d, J = 3.6 Hz, 1H), 7.73 (d, J = 7.6 Hz, 1H), 7.59-7.56 (dd, J = 8.8 Hz, |
| | 1H), 7.49 (d, J = 2.8 Hz, 1H), 7.43 (s, 1H), 7.35-7.32 (t, 3H), 4.36-4.31 (m, 2H), |
| | 4.08 (d, J = 4.4 Hz, 2H), 3.71 (d, J = 13.6 Hz, 1H), 3.62 (d, J = 14 Hz, 1H), 2.73 |
| | (s, 4H), 2.54-2.49 (q, 3H), 2.32-2.24 (m, 2H), 2.16 (s, 1H), 1.97 (d, J = 11.2 Hz, |
| | 1H), 1.70-1.66 (t, 1H), 1.58 (s, 3H). |
| 197 | 1H-NMR: (400 MHz, DMSO-d6): δ: 13.41 (bs, 1H), 8.73 (s, 1H), 7.58 (dd, 8.8, |
| | 2.4 Hz, 1H), 7.44 (d, J = 2.8 Hz, 1H), 7.41 (s, 1H), 7.38 (dd, 4.8, 1.2 Hz, 1H), |
| | 7.32 (d, J = 8.8 Hz, 1H), 6.97-6.93 (m, 2H), 4.36-4.29 (m, 2H), 4.07-4.05 |
| | (m, 2H), 3.90-3.80 (m, 2H), 2.81-2.77 (m, 1H), 2.73 (s, 3H), 2.53-2.39 (m, |
| | 3H), 2.30-2.25 (m, 1H), 2.21(s, 3H), 1.93-1.90 (m, 1H), 1.70-1.61 (m, 1H), |
| | 1.56 (s, 3H). |
| 198 | 1H-NMR: (400 MHz, DMSO-d6): δ: 13.67 (s, 1H), 8.78 (s, 1H), 8.69-8.68 (d, |
| | J = 4.8 Hz, 1H), 7.82 (s, 1H), 7.69-7.68 (d, J = 4.8 Hz, 1H), 7.59 (d, , J = 2.4, Hz, |
| | 1H), 7.57-7.56 (dd, J = 2.4 Hz, 1H), 7.44-7.42 (m, 2H), 7.34-7.32 (d, J = 8.8, |
| | 1H), 4.34-4.31 (m, 2H), 4.07-4.06 (m, 2H), 2.73 (s, 3H), 2.57-2.49 (m, 3H), |
| | 2.28-2.24 (m, 2H), 2.21 (s, 3H), 1.97-1.94 (m, 2H), 1.75-1.64 (m, 1H), 1.58 (s, 3H). |
| 199 | 1H-NMR: (400 MHz, DMSO-d6): δ: 12.82 (br. s, exchange with D2O, 1H), |
| | 8.77 (s, 1H), 8.69 (d, J = 1.2 Hz, 1H), 8.55-8.54 (m, 1H), 8.50 (d, J = 2.8 Hz, |
| | 1H), 7.58 (dd, J = 2.8 Hz, 2.4 Hz, 1H), 7.44-7.42 (m, 2H), 7.33 (d, J = 8.8 Hz, |
| | 1H), 4.34-4.31 (m, 2H), 4.07 (brs, 2H), 3.87-3.76 (m, 2H) 2.78-2.73 (m, 4H), |
| | 2.53-2.46 (m, 4H), 2.32-2.23 (m, 4H), 1.97-1.94 (m, 2H), 1.73-1.64 (m, 1H), |
| | 1.58 (s, 3H). |
| 200 | 1H-NMR: (400 MHz, DMSO-d6): δ 13.68 (s, 1H), 8.79 (s, 1H), 8.48 (s, 1H), |
| | 7.76 (s, 1H), 7.58 (dd, J = 2.80, 8.80 Hz, 1H), 7.48-7.44 (m, 3H), 7.33 (d, J = |
| | 8.80 Hz, 1H), 4.37-4.30 (m, 2H), 4.07-4.03 (m, 2H), 3.76-3.73 (m, 2H), 2.74 (s, |
| | 3H), 2.51-2.50 (m, 2H), 2.50-2.49 (m, 2H), 2.24-2.09 (m, 4H), 1.70-1.63 (m, |
| | 1H), 1.58 (s, 3H), 1.37-1.24 (m, 2H). |
| 201 | 1H-NMR: (400 MHz, DMSO-d6):: 8.77 (s, 1H), 7.68 (dd, J = 7.60, Hz, 2H), |
| | 7.57 (d, J = 2.80 Hz, 1H), 7.56 (d, J = 4.00 Hz, 1H), 7.44 (d, J = 2.80 Hz, 1H), |
| | 7.40 (d, J = −20.80 Hz, 2H), 7.33 (dd, J = 8.80, Hz, 1H), 4.30-4.36 (m, 2H), |
| | 0.00 (d, J = 0.00 Hz, 2H), 3.77 (d, J = 14.00 Hz, 2H), 2.84 (m, 1H), 2.73 (s, |
| | 3H), 2.53 (t, J = 1.60 Hz, 2H), 2.49 (d, J = 0.00 Hz, 1H), 2.33 (t, J = 2.00 Hz, |
| | 2H), 2.29 (d, J = 11.20 Hz, 2H), 1.97 (d, J = 10.40 Hz, 1H), 1.69 (d, J = 8.80 |
| | Hz, 2H), 1.59 (s, 3H), |
| 202 | 1H-NMR: (400 MHz, DMSO-d6):: 8.79 (s, 1H), 8.76 (d, J = -5.20 Hz, 2H), 7.58 |
| | (dd, J = 2.80, 8.80 Hz, 1H), 7.44 (d, J = Hz, 1H), 7.42 (s, 1H), 7.37 (t, J = 4.80 |
| | Hz, 1H), 7.33 (dd, J = 8.80, Hz, 1H), 4.30-4.36 (m, 2H), 4.05-4.08 (m, 2H), |
| | 3.87 (dd, J = 6.40, Hz, 2H), 2.73-2.80 (m, 1H), 2.68 (s, 3H), 2.51 (t, J = 2.00 |
| | Hz, 2H), 2.43 (m, 1H), 2.33 (m, 3H), 1.92 (d, J = 14.80 Hz, 1H), 1.68-1.71 (m, |
| | 1H), 1.57 (s, 3H), (d, J = Hz, 1H), (d, J = Hz, 1H), |
| 203 | 1H-NMR: (400 MHz, DMSO-d6): δ: 8.72 (s, 1H), 7.57 (dd, J = 8.8, 2.8 Hz, |
| | 1H), 7.44 (d, J = 2.8 Hz, 1H), 7.40 (s, 1H), 7.32 (d, J = 8.8 Hz, 1H), 4.39-4.28 |
| | (m, 1H), 4.10-4.06 (m, 2H), 3.16-3.13 (m, 1H), 2.72 (s, 3H), 2.68-2.41 (m, |
| | 7H), 2.34-2.31 (m, 1H), 2.22-2.11 (m, 3H), 2.06 (s, 3H), 2.01-1.91 (m, 2H), |
| | 1.80-1.71 (m, 2H), 1.59 (s, 3H), 1.57-1.55 (m, 2H). |
| 204 | 1H-NMR: (400 MHz, DMSO-d6): δ: 13.1 (br, s, 1H), 8.79 (s, 1H), 8.17 (s, 1H), |
| | 7.59-7.56 (dd, J = 8.8 Hz, 1H), 7.45-7.42 (m, 2H), 7.34-7.31 (d, J = 9.2 Hz , 1H), |
| | 4.32-4.30 (m, 2H), 4.07-4.06 (m, 2H), 2.73 (s, 3H), 2.50-2.45 (m, 7H), 2.24- |
| | 2.11 (m, 6H), 1.90-1.70 (m, 2H), 1.60 (m, 4 H). |
| 205 | 1H-NMR: (400 MHz, DMSO-d6): δ 8.77 (s, 1H), 7.58 (dd, J = 2.40, 9.00 Hz, |
| | 1H), 7.44 (t, J = 7.60 Hz, 1H), 7.33 (d, J = 8.80 Hz, 1H), 4.34-4.31 (m, 2H), |
| | 4.07-4.05 (m, 2H), 2.73 (s, 2H), 2.68-2.67 (m, 2H), 2.51-2.43 (m, 6H), 2.28 (s, |
| | 3H), 2.15-2.08 (m, 1H), 1.86-1.83 (m, 1H), 1.71 (s, 6H), 1.57 (s, 3H), 1.53-1.51 |
| | (m, 2H). |
| 206 | 1H-NMR: (400 MHz, DMSO-d6): δ: 12.59 (bs, 1H), 8.74 (bs, 1H), 7.55 (d, 7.6, |
| | 1H), 7.42 (d, J = 2.4 Hz, 1H), 7.31 (d, J = 8.8 Hz, 1H), 7.33 (d, J = 8.8 Hz, 1H), |
| | 4.33-4.31 (m, 2H), 4.20 (s, 1H), 4.09-4.07 (m, 2H), 2.90-2.82 (m, 1H), |
| | 2.68-2.56 (m, 7H), 2.41-2.21 (m, 6H), 1.97-1.51 (m, 9H), 1.46-1.36 (m, |
| | 4H), 1.10 (s, 3H). |
| 207 | 1H-NMR: (400 MHz, DMSO-d6): δ: 12.59 (bs, 1H), 8.74 (bs, 1H), 7.55 (d, 7.6, |
| | 1H), 7.42 (d, J = 2.4 Hz, 1H), 7.31 (d, J = 8.8 Hz, 1H), 7.33 (d, J = 8.8 Hz, 1H), |
| | 4.33-4.31 (m, 2H), 4.20 (s, 1H), 4.09-4.07 (m, 2H), 2.90-2.82 (m, 1H), |
| | 2.68-2.56 (m, 7H), 2.41-2.21 (m, 6H), 1.97-1.51 (m, 9H), 1.46-1.36 (m, |
| | 4H), 1.10 (s, 3H). |
| 208 | 1H-NMR: (400 MHz, DMSO-d6): δ 8.83 (s, 1H), 7.59 (dd, J = 2.40, 9.00 Hz, |
| | 1H), 7.45 (d, J = 2.00 Hz, 2H), 7.33 (d, J = 9.20 Hz, 1H), 4.62 (brs, 1H), 4.36- |
| | 4.32 (m, 2H), 4.10-4.08 (m, 2H), 3.57 (s, 1H), 3.50-3.44 (m, 4H), 3.33-2.97 (m, |
| | 5H), 2.74 (s, 3H), 2.54-2.49 (m, 3H), 2.20-2.04 (m, 1H), 2.04-1.75 (m, 6H), |
| | 1.56 (s, 3H). |
| 209 | 1H-NMR: (400 MHz, DMSO-d6):: 8.42 (s, 1H), 8.34 (s, 2H), 7.55 (dd, J = 2.80, |
| | 8.80 Hz, 1H), 7.40 (d, J = 2.40 Hz, 1H), 7.30 (d, J = 9.20 Hz, 1H), 7.23 (s, 1H), |
| | 4.26-4.31 (m, 2H), 4.08 (d, J = 4.40 Hz, 3H), 3.60 (m, 1H), 3.59 (d, J = 6.40 Hz, |
| | 2H), 3.33 (s, 3H), 2.68 (s, 2H), 2.32-2.34 (m, 2H), 1.88 (t, J = 12.80 Hz, 1H), |
| | 1.62 (d, J = 9.20 Hz, 3H), 1.49 (m 4H), 1.11 (m, 4H), |
| 210 | 1H-NMR: (400 MHz, DMSO-d6): δ: 8.38 (s, 1H), 7.56-7.53 (m, 1H), 7.40 (d, |
| | J = 2.4 Hz, 1H), 7.30 (d, J = 9.2 Hz, 1H), 7.10 (s, 1H), 4.77 (s, 1H), 4.29 (s, 2H), |
| | 4.07 (s, 2H), 2.67 (s, 3H), 2.50-2.46 (m, 3H), 2.33-2.32 (m, 4H), 2.17-2.07 |
| | (m, 4H), 2.01-1.97 (m, 2H), 1.91-1.83 (m, 2H), 1.61-1.49 (m, 4H), 1.23 (s, 2H), |
| | 1.20 (s, 3H). |
| 211 | 1H-NMR: (400 MHz, DMSO-d6): δ: 8.09 (s, 1H), 7.55-7.53 (m, 1H), 7.37 (d, |
| | J = 2.8 Hz, 1H), 7.26 (d, J = 8.8 Hz, 1H), 7.11 (s, 1H), 4.28 (s, 2H), 4.09 (s, 2H), |
| | 2.65 (s, 3H), 2.50-2.46 (m, 4H), 2.23-2.12 (m, 4H), 2.07-1.81 (m, 4H), 1.69- |
| | 1.59 (m, 5H), 1.55-1.45 (m, 2H), 1.23 (s, 6H). |
| 212 | 1H-NMR: (400 MHz, DMSO-d6: δ 8.77 (s, 1H), 7.58 (dd, J = 2.80, 9.00 Hz, |
| | 1H), 7.44 (d, J = 2.80 Hz, 1H), 7.41 (s, 1H), 7.33 (d, J = 8.80 Hz, 1H), 6.34 (s, |
| | 1H), 4.32 (t, J = 2.40 Hz, 2H), 4.07 (t, J = 4.40 Hz, 2H), 2.73 (s, 3H), 2.51 (m, |
| | 2H), 2.45-2.42 (m, 6H), 2.22-2.15 (m, 1H), 2.09 (d, J = 1.60 Hz, 2H), 1.84 (d, |
| | J = 13.20 Hz, 3H), 1.69 (m, 3H), 1.58 (s, 3H), 1.48 (m, 3H). |
| 213 | 1H-NMR: (400 MHz, DMSO-d6): 8.52 (s, 1H), 7.56 (dd, J = 2.80, 9.00 Hz, 1H), |
| | 7.42 (d, J = 2.80 Hz, 1H), 7.32-7.30 (m, 2H), 4.33-4.31 (m, 2H), 4.08-4.06 (m, |
| | 2H), 3.17-3.12 (m, 1H), 2.69 (s, 3H), 2.66-2.62 (m, 3H), 2.51-2.49 (m, 2H), |
| | 2.32-2.26 (m, 3H), 2.27 (s, 3H), 1.93-1.83 (m, 3H), 1.63 (s, 3H), 1.58-1.55 (m, |
| | 1H), 1.07-1.03 (m, 2H). |
| 214 | 1H-NMR: (400 MHz, DMSO-d6): δ 8.50 (s, 1H), 8.15 (s, 1H), 7.51 (dd, J = |
| | 2.40, 9.00 Hz, 1H), 7.39 (d, J = 2.80 Hz, 1H), 7.27 (d, J = 8.80 Hz, 1H), 7.06 (s, |
| | 1H), 6.05-5.89 (m, 1H), 4.27 (t, J = 5.20 Hz, 2H), 4.09 (t, J = 4.80 Hz, 2H), 3.93 |
| | (s, 1H), 3.27-3.23 (m, 1H), 3.06-2.98 (m, 6H), 2.90 (s, 3H), 2.79-2.63 (m, 1H), |
| | 2.63 (t, J = Hz, 3H), 2.52-2.51 (m, 1H), 2.50-2.49 (m, 2H), 1.86-1.76 (m, 2H), |
| | 1.70-1.53 (m, 7H), 1.38-1.49 (m, 2H), 1.27 (t, J = 13.60 Hz, 2H). |
| 215 | 1H-NMR: (400 MHz, DMSO-d6): δ 8.49 (s, 1H), 7.55 (dd, J = 2.80, 9.00 Hz, |
| | 1H), 7.39 (d, J = 9.20 Hz, 1H), 7.31-7.24 (m, 2H), 4.29 (t, J = 4.80 Hz, 2H), |
| | 4.09 (t, J = 4.40 Hz, 2H), 3.97 (s, 1H), 3.23-3.16 (m, 4H), 2.89 (s, 1H), 2.67 (s, |
| | 3H), 2.65-2.61 (m, 2H), 2.45 (m, 1H), 2.33-2.11 (m, 4H), 1.89 (t, J = 10.40 Hz, |
| | 1H), 1.67-1.54 (m, 8H), 1.46 (m, 2H), 1.33-1.24 (m, 2H), 1.11 (s, 3H). |
| 216 | 1H-NMR: (400 MHz, DMSO-d6): δ: 13.05 (bs, 1H), 8.93 (s, 1H), 8.07 (d, J = |
| | 5.2 Hz, 1H), 7.60-7.57 (m, 1H), 7.47 (s, 1H), 7.44 (d, J = 2.4 Hz, 1H), 7.34 (d, |
| | J = 8.8, 1H), 6.95 (d, J = 5.2 Hz, 1H), 6.76 (s, 1H), 4.35-4.31 (m, 2H), 4.09- |
| | 4.06 (m, 2H), 3.82 (s, 3H), 3.68-3.55 (m, 2H), 3.50 (s, 3H), 2.73 (s, 3H), 2.61- |
| | 2.41 (m, 3H), 2.33-2.23 (m, 4H), 1.95-1.92 (m, 1H), 1.64-1.60 (m, 4H). |
| 217 | 1H-NMR: (400 MHz, DMSO-d6): δ: 13.65 (bs, 1H), 8.62 (s, 1H), 8.07 (d, J = |
| | 5.2 Hz, 1H), 7.56 (dd, 8.8, 2.8 Hz, 1H), 7.43 (d, J = 2.4 Hz, 1H), 7.35 (s, 1H), |
| | 7.32 (d, J = 8.8 Hz, 1H), 6.96 (dd, J = 5.6, 1.2 Hz, 1H), 6.75 (s, 1H), 4.35-4.27 |
| | (m, 2H), 4.09-4.06 (m, 2H), 3.82 (s, 3H), 3.67-3.51 (m, 2H), 2.79-2.73 (m, |
| | 1H), 2.72 (s, 3H), 2.55-2.46 (m, 3H), 2.33-2.16 (m, 4H), 1.95-1.92 (m, |
| | 1H), 1.69-1.61 (m, 4H). |
| 218 | 1H-NMR: (400 MHz, DMSO-d6): δ: 13.51 (bs, 1H), 8.65 (s, 1H), 8.07 (d, J = |
| | 5.2 Hz, 1H), 7. 57 (dd, 8.8, 2.8 Hz, 1H), 7.43 (d, J = 2.4 Hz, 1H), 7.37 (s, 1H), |
| | 7.32 (d, J = 9.2, 1H), 6.95 (d, J = 5.2 Hz, 1H), 6.75 (s, 1H), 4.35-4.29 (m, 2H), |
| | 4.09-4.06 (m, 2H), 3.81 (s, 3H), 3.67-3.55 (m, 2H), 2.72 (s, 3H), 2.61-2.46 |
| | (m, 3H), 2.33-2.16 (m, 4H), 1.95-1.92 (m, 1H), 1.66-1.60 (m, 4H). |
| 219 | 1H-NMR: (400 MHz, DMSO-d6): δ 8.65 (d, J = 4.80 Hz, 1H), 7.96 (d, J = 0.80 |
| | Hz, 1H), 7.54 (dd, J = 1.60, 5.00 Hz, 1H), 7.48 (dd, J = 2.80, 8.80 Hz, 1H), 7.43 |
| | (d, J = 2.80 Hz, 1H), 7.25 (d, J = 9.20 Hz, 1H), 4.31 (m, 2H), 4.23-4.22 (m, 2H), |
| | 2.61-2.68 (m, 7H), 2.53 (d, J = 3.20 Hz, 2H), 2.33-2.22 (m, 7H), 2.18 (m, 3H), |
| | 1.93-1.91 (m, 1H), 1.59 (m, 1H), |
| 220 | 1H-NMR: (400 MHz, DMSO-d6): δ: 13.02 (bs, 1H), 8.94 (s, 1H), 8.07 (bs, 1H), |
| | 7.60 (dd, J = 8.8, 2.8 Hz, 1H), 7.49 (s, 1H), 7.45 (d, J = 2.8 Hz, 1H), 7.35 (d, J = |
| | 8.8 Hz, 1H), 7.20-6.80 (m, 2H), 4.35-4.30 (m, 2H), 4.09-4.03 (m, 2H), 3.84 |
| | (s, 3H), 3.68-3.52 (m, 4H), 2.73 (s, 5H), 2.61-2.41 (m, 4H), 2.33-1.83 (m, |
| | 4H), 1.62-1.54 (m, 4H). |
| 221 | 1H-NMR: (400 MHz, DMSO-d6): 8.34 (s, 1H), 7.56 (dd, J = 2.80, 9.00 Hz, 1H), |
| | 7.42 (d, J = 2.80 Hz, 1H), 7.32-7.22 (m, 2H), 4.30-4.28 (m, 2H), 4.09-4.08 (m, |
| | 2H), 3.21-3.14 (m, 1H), 2.67 (s, 3H), 2.61-2.59 (m, 2H), 2.50-2.49 (m, 3H), |
| | 2.27 (s, 3H), 2.18-2.15 (m, 3H), 1.91-1.86 (m, 3H), 1.65 (s, 3H), 1.58-1.55 (m, |
| | 1H), 1.07-1.03 (m, 2H). |
| 222 | 1H-NMR: (400 MHz, , DMSO-d6): δ 13.59 (s, 1H), 8.75 (s, 1H), 7.58 (dd, J = |
| | 2.80, 8.80 Hz, 1H), 7.42-7.47 (m, 3H), 7.31-7.38 (m, 3H), 7.21 (d, J = 8.00 Hz, |
| | 1H), 4.33 (t, J = 5.20 Hz, 2H), 4.08 (t, J = 4.00 Hz, 2H), 3.74-3.62 (m, 2H), 2.76 |
| | (m, 1H), 2.73 (s, 3H), 2.55-2.54 (m, 1H), 2.50-2.49 (m, 2H), 2.30-2.27 (m, 1H), |
| | 2.18 (s, 3H), 1.96-1.93 (m, 1H), 1.69-1.65 (m, 1H), 1.59 (s, 3H), |
| 223 | 1H-NMR: (400 MHz, DMSO-d6): δ: 8.78 (s, 1H), 7.58 (dd, J = 8.8, 2.8 Hz, |
| | 1H), 7.44 (d, J = 2.8 Hz, 1H), 7.42 (s, 1H), 7.33 (d, J = 8.8 Hz, 1H), 4.35-4.28 |
| | (m, 2H), 4.10-4.06 (m, 2H), 3.09-2.95 (m, 1H), 2.73 (s, 3H), 2.59-2.56 (m, |
| | 1H), 2.55-2.42 (m, 6H), 2.22-2.09 (m, 6H), 1.84-1.50 (m, 7H). |
| 224 | 1H-NMR: (400 MHz, DMSO-d6): δ 8.08 (s, 1H), 7.52 (dd, J = 2.40, 8.80 Hz, |
| | 1H), 7.38 (d, J = 2.40 Hz, 1H), 7.27 (d, J = 9.20 Hz, 1H), 7.09 (s, 1H), 4.27 (t, |
| | J = 4.0 Hz, 2H), 4.08 (t, J = 4.0 Hz, 2H), 3.97 (s, 1H), 2.79 (m, 1H), 2.62 (s, 3H), |
| | 2.49-2.47 (m, 2H), 2.20 (s, 3H), 1.85-1.78 (m, 1H), 1.69-1.63 (m, 4H), 1.59- |
| | 1.53 (m, 3H), 1.43-1.37 (m, 2H), 1.30-1.23 (m, 3H), 1.09-1.06 (m, 6H). |
| 225 | 1H-NMR: (400 MHz, DMSO-d6): δ 7.98 (s, 1H), 7.49 (dd, J = 2.80, 8.80 Hz, |
| | 1H), 7.36 (d, J = 2.40 Hz, 1H), 7.25 (d, J = 8.80 Hz, 1H), 7.01 (s, 1H), 4.27-4.26 |
| | (m, 2H), 4.09 (m, 2H), 3.16 (s, 1H), 2.79 (m, 1H), 2.16 (s, 3H), 2.46-2.43 (m, |
| | 2H), 1.89 (s, 3H), 1.85-1.82 (m, 1H), 1.72-1.63 (m, 4H), 1.60-1.53 (m, 3H), |
| | 1.40 (m, 3H), 1.30-1.23 (m, 3H), 1.14-1.06 (m, 5H). |
| 226 | 1H-NMR: (400 MHz, DMSO-d6): δ: 8.77 (s, 1H), 7.58 (dd, J = 8.8 Hz, 2.8, |
| | 1H), 7.44 (d, J = 2.4 Hz, 1H), 7.42 (s, 1H), 7.33 (d, J = 8.8 Hz, 1H), 4.36-4.12 |
| | (m, 5H), 3.50 (s, 3H), 2.82-278 (m, 1H), 2.67 (s, 3H), 2.54-2.40 (m, 3H), |
| | 2.20 (s, 3H), 1.73-1.67 (s, 1H), 1.70-1.54 (m, 8H), 1.41-1.36 (m, 4H), 1.23 |
| | (s, 1H), 1.13 (s, 3H). |
| 227 | 1H-NMR: (400 MHz, DMSO-d6): δ: 7.92 (s, 1H), 7.51 (dd, J = 8.8 Hz, 2.8, |
| | 1H), 7.36 (d, J = 2.4 Hz, 1H), 7.26 (d, J = 9.2 Hz, 1H), 7.03 (s, 1H), 4.26-4.09 |
| | (m, 5H), 2.82-278 (m, 1H), 2.67 (s, 3H), 2.44-2.40 (m, 2H), 2.17 (s, 4H), |
| | 1.73-1.67 (s, 3H), 1.60-1.54 (m, 6H), 1.41-1.31 (m, 4H), 1.23 (s, 2H), 1.11 |
| | (s, 3H) |
| 228 | 1H-NMR: (400 MHz, DMSO-d6): δ 8.74 (brs, 1H), 7.57 (dd, J = 2.8, 9.2 Hz, |
| | 1H), 7.44 (d, J = 2.4 Hz, 1H), 7.41 (br, 1H), 7.34 (d, J = 8.8 Hz, 1H), 4.83 (s, |
| | 1H), 4.33-4.29 (m, 2H), 4.06 (s, 2H), 2.72 (s, 3H), 2.67 (s, 2H), 2.48-2.40 |
| | (m, 1H), 2.38-2.28 (m, 2H), 2.15-2.10 (m, 1H), 2.06 (s, 5H), 1.90-1.86 (m, |
| | 2H), 1.80-1.76 (m, 1H), 1.61 (s, 3H), 1.57-1.51 (m, 1H), 1.19 (s, 3H). |
| 229 | 1H-NMR: (400 MHz, DMSO-d6): δ 8.63 (br, 1H), 7.57 (d, J = 6.8 Hz, 1H), 7.43 |
| | (d, J = 2.0 Hz, 1H), 7.35 (br, 1H), 7.32 (d, J = 9.2 Hz, 1H), 4.83 (s, 1H), 4.30 |
| | (s, 2H), 4.06 (s, 2H), 2.70 (s, 3H), 2.50 (s, 2H), 2.40 (s, 1H), 2.35-2.31 (m, |
| | 2H), 2.15-2.10 (m, 1H), 2.06 (s, 5H), 1.90-1.86 (m, 2H), 1.80-1.76 (m, |
| | 1H), 1.61 (s, 3H), 1.57-1.51 (m, 1H), 1.19 (s, 3H). |
| 231 | 1H-NMR: (400 MHz, DMSO-d6): δ: 13.04 (s, 1H), 8.96-8.86 (m, 2H), 7.61 |
| | (dd, J = 9.2, 2.8 Hz, 1H), 7.51 (s, 1H), 7.46-7.45 (m, 1H), 7.35 (d, J = 9.2 Hz, |
| | 1H), 4.41-4.31 (m, 2H), 4.15-4.07 (m, 2H), 3.72-3.65 (m, 2H), 3.52 (s, 3H), |
| | 3.27-3.21 (m, 1H), 2.97-2.90 (m, 4H), 2.75 (s, 3H), 2.53-2.29 (m, 3H), 2.05- |
| | 1.60 (m, 10H), 1.83-1.59 (m, 3H), 0.87-0.83 (m, 3H). |
| 232 | 1H-NMR: (400 MHz, DMSO-d6): δ: 13.04 (s, 1H), 8.96-8.86 (m, 2H), 7.61 |
| | (dd, J = 9.2, 2.8 Hz, 1H), 7.51 (s, 1H), 7.46-7.45 (m, 1H), 7.35 (d, J = 9.2 Hz, |
| | 1H), 4.37-4.31 (m, 2H), 4.15-4.07 (m, 2H), 3.67-3.65 (m, 2H), 3.52 (s, 3H), |
| | 3.27-3.21 (m, 2H), 2.97-2.90 (m, 4H), 2.67 (s, 3H), 2.33-2.27 (m, 1H), 1.99- |
| | 1.90 (m, 2H), 1.83-1.59 (m, 8H), 1.23-1.14 (m, 3H), 0.87-0.83 (m, 3H). |
| 233 | 1H NMR: (400 MHz, DMSO-d6) 8: 13.73 (br. s, exchange with D20, 1H), 12.27 |
| | (br. s, exchange with D20, 1H)) 8.81 (s, 1H), 7.91 (s, 1H), 7.42 (s, 1H), 4.60- |
| | 4.50 (m, 2H), 4.17-4.09 (m, 2H), 2.71-2.65 (m, 4H), 2.54 (br. s, 1H), 2.50-2.45 |
| | (m, 2H, merged with DMSO-d6), 2.36-2.29 (m, 4H), 1.96-1.90 (m, 1H), 1.86- |
| | 1.84 (m, 1H), 1.73 (s, 3H), 1.68-1.61 (m, 1H), 0.47-0.46 (m, 2H), 0.31-0.311 |
| | (m, 2H). |
| 234 | 1H NMR: (400 MHz, DMSO-d6) 8: 13.65 (br. s, exchange with D20, 1H), 8.81 |
| | (s, 1H), 7.91 (s, 1H), 7.42 (s, 1H), 4.59-4.51 (m, 2H), 4.15-4.11 (m, 2H), 2.71- |
| | 2.65 (m, 4H), 2.51 (br. s, 1H), 2.50-2.45 (m, 2H, merged with DMSO-d6), 2.36- |
| | 2.29 (m, 4H), 1.96-1.93 (m, 1H), 1.86-1.79 (m, 1H), 1.73 (s, 3H), 1.66-1.62 (m, |
| | 1H), 0.48-0.46 (m, 2H), 0.32-0.31 (m, 2H). |
| 235 | 1H-NMR: (400 MHz, DMSO-d6): δ 8.70 (s, 1H), 7.58 (dd, J = 2.80, 8.80 Hz, |
| | 1H), 7.46-7.42 (m, 2H), 7.39 (s, 1H), 7.32 (d, J = 8.80 Hz, 1H), 7.25 (d, J = 1.60 |
| | Hz, 1H), 6.34 (d, J = 9.60 Hz, 1H), 4.34-4.31 (m, 2H), 4.08 (m, 2H), 3.41-3.30 |
| | (m, 2H), 2.73-2.69 (m, 4H), 2.53-2.51 (m, 4H), 2.38-2.35 (m, 1H), 2.26 (s, 3H), |
| | 1.93-1.90 (m, 1H), 1.66 (s, 4H). |
| 236 | 1H-NMR: (400 MHz, DMSO-d6): δ 8.76 (s, 1H), 7.58 (dd, J = 2.40, 9.00 Hz, |
| | 1H), 7.44 (d, J = 2.40 Hz, 1H), 7.42 (s, 1H), 7.40 (d, J = 2.80 Hz, 1H), 7.38 (d, |
| | J = 2.40 Hz, 1H), 7.33 (d, J = 4.00 Hz, 1H), 7.21 (s, 1H), 6.29 (d, J = 9.20 Hz, |
| | 1H), 4.33-4.28 (m, 2H), 4.11-4.07 (m, 2H), 3.36 (m, 2H), 2.73-2.67 (m, 4H), |
| | 2.46 (s, 3H), 2.33-2.32 (m, 1H), 2.26 (s, 3H), 1.91-1.89 (m, 1H), 1.67 (s, 4H). |
| 237 | 1H NMR: (400 MHz, CDCl3) δ: 8.74 (s, 1H), 7.48 (s, 1H), 7.27 (s, 1H), 4.45 (t, |
| | J = 6.4 Hz, 2H), 4.12 (t, J = 4.8 Hz, 2H), 2.88 (q, J = 7.2 Hz, 2H), 2.67-2.60 (m, |
| | 7H), 2.49-2.40 (m, 2H), 2.38-2.29 (m, 6H), 1.91 (s, 3H), 1.87-1.81 (m, 1H), |
| | 1.59-1.54 (m, 1H), 1.16 (t, 3H). |
| 238 | 1H NMR (400 MHz, CDCl3) δ: 8.74 (s, 1H), 7.48 (s, 1H), 7.27 (s, 1H), 4.45 (t, |
| | J = 6.4 Hz, 2H), 4.12 (t, J = 4.8 Hz, 2H), 2.88 (q, J = 7.2 Hz, 2H), 2.67-2.60 (m, |
| | 7H), 2.49-2.40 (m, 2H), 2.38-2.29 (m, 6H), 1.91 (s, 3H), 1.87-1.81 (m, 1H), |
| | 1.59-1.54 (m, 1H), 1.16 (t, 3H). |
| 239 | 1H-NMR: (400 MHz, DMSO-d6): δ 8.87 (s, 1H), 7.58 (dd, J = 2.80, 8.80 Hz, |
| | 1H), 7.43 (d, J = 2.40 Hz, 2H), 7.34 (d, J = 8.80 Hz, 1H), 4.33-4.32 (m, 2H), |
| | 4.08-4.06 (m, 2H), 3.42 (s, 3H), 3.32 (s, 4H), 2.90-2.88 (m, 2H), 2.72 (s, |
| | 3H), 2.21-2.17 (m, 1H), 2.49 (m, 5H), 2.32-2.22 (m, 1H), 1.91-1.87 (m, 3H), |
| | 1.62 (s, 4H), 1.46-1.42 (m, 2H). |
| 240 | 1H-NMR: (400 MHz, DMSO-d6): δ 8.84 (s, 1H), 7.58 (dd, J = 2.80, 8.80 Hz, |
| | 1H), 7.43-7.41 (m, 2H), 7.32 (d, J = 6.40 Hz, 1H), 4.34-4.31 (m, 2H), 4.08-4.06 |
| | (m, 2H), 3.40 (s, 3H), 3.23-3.19 (m, 3H), 2.98 (m, 1H), 2.86-2.81 (m, 2H), 2.72 |
| | (s, 3H), 2.62 (m, 1H), 2.40-2.35 (m, 5H), 2.24-2.17 (m, 1H), 1.91-1.87 (m, |
| | 3H), 1.62-1.59 (m, 4H), 1.65-1.51 (m, 2H). |
| 241 | 1H-NMR 400 MHz, DMSO-d6): δ 8.79 (s, 1H), 7.59-7.57 (m, 1H), 7.43-7.23 |
| | (m, 1H), 7.39 (s, 1H), 7.33 (d, J = 8.80 Hz, 1H), 4.32 (t, J = 4.80 Hz, 2H), 4.08 |
| | (t, J = 4.40 Hz, 2H), 3.38 (s, 3H), 2.71-2.67 (m, 8H), 2.55-2.54 (m, 3H), 2.33- |
| | 2.27 (m, 1H), 1.94-1.91 (m, 1H), 1.65-1.57 (m, 8H), 1.45-1.43 (m, 2H). |
| 242 | 1H-NMR: (400 MHz, DMSO-d6): δ: 8.60 (s, 1H), 7.57 (dd, J = 8.8, 2.8 Hz, |
| | 1H), 7.43 (d, J = 2.8 Hz, 1H), 7.36 (s, 1H), 7.32 (d, J = 8.8 Hz, 1H), 4.35-4.26 |
| | (m, 2H), 4.08-4.04 (m, 2H), 3.02-2.98 (m, 1H), 2.85-2.71 (m, 6H), 2.49- |
| | 2.23 (m, 7H), 2.04-1.96 (m, 1H), 1.85-1.70 (m, 3H), 1.58-1.41 (m, 7H), |
| | 1.25-1.10 (m, 6H), 0.55-0.49 (m, 2H), 0.39-0.29 (m, 2H). |
| 243 | 1H-NMR: (400 MHz, DMSO-d6): δ: 8.65 (s, 1H), 7.57 (dd, J = 8.8, 2.8 Hz, |
| | 1H), 7.43 (d, J = 2.8 Hz, 1H), 7.38 (s, 1H), 7.32 (d, J = 9.2 Hz, 1H), 4.36-4.26 |
| | (m, 2H), 4.11-4.00 (m, 2H), 3.02-2.98 (m, 1H), 2.85-2.75 (m, 3H), 2.71 (s, |
| | 3H), 2.49-2.23 (m, 8H), 2.04-1.96 (m, 1H), 1.85-1.70 (m, 3H), 1.60-1.41 |
| | (m, 7H), 1.25-1.10 (m, 6H), 0.58-0.49 (m, 2H), 0.39-0.29 (m, 2H). |
| 244 | 1H-NMR 400 MHz, DMSO-d6): δ 7.91 (s, 1H), 7.54 (dd, J = 2.80, 9.00 Hz, |
| | 1H), 7.41 (d, J = 2.80 Hz, 1H), 7.28 (d, J = 8.80 Hz, 1H), 4.30-4.28 (m, 2H), |
| | 4.12-4.10 (m, 2H), 3.72 (s, 3H), 2.70-2.67 (m, 5H), 2.33-2.21 (m, 6H), 1.94- |
| | 1.89 (m, 5H), 1.56-1.55 (m, 1H), 0.45-0.28 (m, 4H). |
| 245 | 1H-NMR: (400 MHz, DMSO-d6): δ 8.47 (s, 1H), 7.54 (dd, J = 2.40, 8.80 Hz, |
| | 1H), 7.40 (d, J = 2.80 Hz, 1H), 7.30 (d, J = 9.20 Hz, 1H), 7.22 (s, 1H), 4.30 (t, |
| | J = 4.80 Hz, 2H), 4.08 (t, J = 4.80 Hz, 2H), 3.14 (s, 3H), 2.67 (s, 3H), 2.56-2.52 |
| | (m, 6H), 2.45-2.41 (m, 2H), 2.23-2.17 (m, 1H), 1.91-1.87 (m, 1H), 1.68 (s, 3H), |
| | 1.60-1.59 (m, 5H), 1.41-1.40 (m, 2H). |
| 246 | 1H-NMR: (400 MHz, DMSO-d6): δ 8.41 (s, 1H), 7.56 (d, J = 2.40 Hz, 1H), |
| | 7.41 (d, J = 2.80 Hz, 1H), 7.31-7.26 (m, 3H), 6.24 (s, 1H), 6.15 (d, J = 6.40 Hz, |
| | 1H), 4.31-4.30 (m, 2H), 4.09-4.08 (m, 2H), 3.49-3.40 (m, 2H), 2.72-2.66 (m, |
| | 6H), 2.52-2.50 (m, 2H), 2.27 (s, 4H), 1.90 (d, J = 15.20 Hz, 1H), 1.63 (s, 4H). |
| 247 | 1H-NMR: (400 MHz, DMSO-d6): δ 8.51 (s, 1H), 7.56 (d, J = 11.60 Hz, 1H), |
| | 7.42 (d, J = 2.40 Hz, 1H), 7.31-7.30 (m, 2H), 7.26 (d, J = 6.80 Hz, 1H), 6.25 (s, |
| | 1H), 6.15 (d, J = 8.40 Hz, 1H), 4.31-4.29 (m, 2H), 4.09-4.08 (m, 2H), 3.44-3.40 |
| | (m, 2H), 2.70-2.67 (m, 6H), 2.52-2.51 (m, 2H), 2.32-2.25 (m, 4H), 1.91 (d, J = |
| | 12.80 Hz, 1H), 1.07-1.03 (s, 4H), |
| 248 | 1H-NMR: (400 MHz, DMSO-d6): δ: 13.58 (s, 1H), 8.49 (s, 1H), 8.07 (d, J = 5.2 |
| | Hz, 1H), 7.55 (dd, J = 8.8, 2.4 Hz, 1H), 7.42 (d, J = 2.8 Hz, 1H), 7.32-7.31 (m, |
| | 2H), 6.94 (d, J = 4.0 Hz, 1H), 6.75 (s, 1H), 4.34-4.29 (m, 2H), 4.11-4.07 (m, |
| | 2H), 3.82 (s, 3H), 3.66-3.55 (m, 2H), 2.71-2.55 (m, 4H), 2.69 (s, 3H), 2.30- |
| | 2.21 (m, 1H), 2.18 (s, 3H), 1.95-1.92 (m, 1H), 1.71-1.65 (s, 4H). |
| 249 | 1H-NMR: (400 MHz, DMSO-d6): δ 8.53 (s, 1H), 7.55 (dd, J = 2.80, 9.00 Hz, |
| | 1H), 7.41 (d, J = 2.40 Hz, 1H), 7.32-7.30 (m, 2H), 4.31 (m, 2H), 4.08-4.07 (m, |
| | 2H), 2.70-2.65 (m, 4H), 2.51-2.50 (m, 2H), 2.44 (s, 3H), 2.33-2.16 (m, 4H), |
| | 1.89-1.85 (m, 1H), 1.62 (s, 3H), 1.58-1.49 (m, 4H), 1.09-0.87 (m, 3H). |
| 250 | 1H-NMR: (400 MHz, DMSO-d6): δ 8.61 (s, 1H), 7.56 (dd, J = 2.40, 9.00 Hz, |
| | 1H), 7.42 (d, J = 2.80 Hz, 1H), 7.33-7.30 (m, 2H), 4.31 (m, 2H), 4.08-4.07 (m, |
| | 2H), 2.71-2.65 (m, 4H), 2.51-2.43 (m, 5H), 2.23-2.17 (m, 1H), 1.90-1.85 (m, |
| | 1H), 1.61 (s, 3H), 1.56-1.39 (m, 7H), 1.09-1.05 (m, 3H). |
| 251 | 1H-NMR: (400 MHz, DMSO-d6): δ: 8.57 (s, 1H), 7.56 (dd, J = 8.8, 2.4 Hz, |
| | 1H), 7.41 (d, J = 2.4 Hz, 1H), 7.31 (d, J = 8.8 Hz, 2H), 4.31 (t, J = 4.8 Hz, 2H), |
| | 4.08 (t, J = 4.8 Hz, 2H), 3.22 (s, 3H), 2.72-2.61 (m, 9H), 2.49-2.45 (m, 2H), |
| | 2.22-2.18 (m, 1H), 1.98-1.84 (m, 5H), 1.75 (s, 3H), 1.60-1.51 (m, 1H). |
| 252 | 1H-NMR: (400 MHz, DMSO-d6): δ 7.49 (d, J = 8.40 Hz, 2H), 7.36 (d, J = 2.40 |
| | Hz, 1H), 7.22 (d, J = 9.20 Hz, 1H), 4.27-4.26 (m, 2H), 4.16-4.15 (m, 2H), 3.61- |
| | 3.53 (m, 3H), 2.62-2.51 (m, 5H), 2.51-2.44 (m, 2H), 2.44-2.22 (m, 7H), 2.06 (s, |
| | 3H), 1.91-1.85 (m, 1H), 1.51-1.48 (m, 1H), 1.23 (s, 1H), 1.07 (t, J = 7.20 Hz, |
| | 1H), 1.05-0.91 (m, 2H). |
| 253 | 1H-NMR: 400 MHz, DMSO-d6): δ 7.50 (d, J = 8.40 Hz, 2H), 7.38 (d, J = 2.40 |
| | Hz, 1H), 7.25 (d, J = 8.80 Hz, 1H), 4.28 (m, 2H), 4.14-4.13 (m, 2H), 3.65 (m, |
| | 3H), 2.65-2.51 (m, 5H), 2.50-2.42 (m, 2H), 2.19-2.02 (m, 5H), 1.93 (s, 3H), |
| | 1.86-1.75 (m, 1H), 1.52-1.32 (m, 3H), 1.00 (t, J = 3.60 Hz, 2H), 0.93-0.83 (m, 2H). |
| 254 | 1H-NMR: (400 MHz, DMSO-d6): δ: 8.32 (s, 1H), 7.54 (dd, J = 8.8, 2.4 Hz, |
| | 1H), 7.39 (d, J = 2.4 Hz, 1H), 7.29 (d, J = 8.8 Hz, 1H), 7.15 (s, 1H), 4.30 (t, J = |
| | 4.8 Hz, 2H), 4.09 (t, J = 4.8 Hz, 2H), 3.22-3.19 (m, 1H), 3.01 (s, 3H), 2.86- |
| | 2.80 (m, 1H), 2.72-2.61 (m, 1H), 2.49-2.45 (m, 1H), 2.22-2.18 (m, 1H), |
| | 1.98-1.84 (m, 5H), 1.67 (s, 3H), 1.60-1.51 (m, 2H). |
| 255 | 1H-NMR: (400 MHz, DMSO-d6): δ: 13.05 (s, 1H), 9.52 (s, 1H), 8.96 (s, 1H), |
| | 7.61 (dd, 8.8, 2.8 Hz, 1H), 7.51 (s, 1H), 7.45 (d, J = 2.8 Hz, 1H), 7.35 (d, J = 9.2 |
| | Hz, 1H), 4.40-4.32 (m, 2H), 4.09-4.05 (m, 3H), 3.51 (s, 3H), 3.50-3.31 (m, |
| | 3H), 2.88-2.73 (m, 9H), 2.65-2.47 (m, 6H), 2.20-2.11 (m, 1H), 1.87-1.74 |
| | (m, 1H), 1.56 (d, J = 2.4 Hz, 1H). |
| 256 | 1H-NMR: (400 MHz, DMSO-d6): δ: 13.05 (s, 1H), 9.60 (s, 1H), 8.96 (s, 1H), |
| | 7.60 (dd, 8.8, 2.8 Hz, 1H), 7.51 (s, 1H), 7.45 (d, J = 2.4 Hz, 1H), 7.35 (d, J = 8.8 |
| | Hz, 1H), 4.40-4.32 (m, 2H), 4.09-4.05 (m, 2H), 3.60-3.31 (m, 6H), 2.90- |
| | 2.71 (m, 9H), 2.65-2.51 (m, 6H), 2.20-2.11 (m, 1H), 1.87-1.74 (m, 1H), |
| | 1.56 (d, J = 2.4 Hz, 1H). |
| 257 | 1H-NMR: (400 MHz, DMSO-d6): δ: 8.28 (s, 1H), 7.54 (dd, J = 2.4 Hz, 1H), |
| | 7.40-7.39 (d, J = 2.4 Hz, 1H), 7.29-7.26 (d, J = 8.8 Hz, 1H), 7.15 (s, 1H), |
| | 4.29-4.28 (m, 2H), 4.09-4.08 (m, 2H), 2.88-2.78 (m, 1H), 2.71-2.58 (m, |
| | 9H), 2.47 (s, 3H), 2.20-2.15 (m, 3H), 1.94-1.90 (m, 2H), 1.61 (s, 3H), 0.48- |
| | 0.46 (m, 2H), 0.29-0.27 (m, 2H) |
| 258 | 1H-NMR: (400 MHz, DMSO-d6): δ: 8.69 (s, 1H), 7.57 (dd, 8.8, 2.4 Hz, 1H), |
| | 7.44 (d, J = 2.4 Hz, 1H), 7.39 (s, 1H), 7.33 (d, J = 8.8 Hz, 1H), 4.35-4.28 (m, |
| | 2H), 4.09-4.05 (m, 2H), 2.71 (s, 3H), 2.70-2.55 (m, 4H), 2.50-2.41 (m, |
| | 4H), 2.35-2.11 (m, 7H), 1.91-1.84 (m, 1H), 1.61-1.51 (m, 4H). |
| 259 | 1H-NMR: (400 MHz, DMSO-d6): δ: 8.58 (s, 1H), 7.56 (dd, 8.8, 2.8 Hz, 1H), |
| | 7.43 (d, J = 2.4 Hz, 1H), 7.34 (s, 1H), 7.31 (d, J = 8.8 Hz, 1H), 4.32-4.28 (m, |
| | 2H), 4.09-4.02 (m, 2H), 2.72 (s, 3H), 2.70-2.55 (m, 4H), 2.53-2.41 (m, |
| | 4H), 2.35-2.11 (m, 7H), 1.91-1.84 (m, 1H), 1.62 (s, 3H), 1.61-1.51 (m, 1H). |
| 260 | 1H-NMR: (400 MHz, DMSO-d6): δ: 13.05 (br, 1H), 9.22-9.10 (m, 1H), 8.95 |
| | (s, 1H), 7.61 (dd, J = 8.8, 2.8 Hz, 1H), 7.51 (s, 1H), 7.46 (d, J = 2.8 Hz, 1H), |
| | 7.36 (d, J = 8.8 Hz, 1H), 4.40-4.33 (m, 2H), 4.12-4.01 (m, 2H), 3.51 (s, 3H), |
| | 3.06-2.75 (m, 7H), 2.67-2.50 (m, 8H), 2.33-2.10 (m, 1H), 1.90-1.80 (m, |
| | 1H), 1.59 (s, 3H), 1.10-0.92 (m, 4H). |
| 261 | 1H-NMR: (400 MHz, DMSO-d6): δ: 13.05 (br, 1H), 9.19-9.10 (m, 1H), 8.95 |
| | (s, 1H), 7.61 (dd, J = 8.8, 2.8 Hz, 1H), 7.51 (s, 1H), 7.46 (d, J = 2.8 Hz, 1H), |
| | 7.36 (d, J = 8.8 Hz, 1H), 4.40-4.33 (m, 2H), 4.12-4.01 (m, 2H), 3.52 (s, 3H), |
| | 3.06-2.75 (m, 7H), 2.67-2.50 (m, 8H), 2.33-2.20 (m, 1H), 1.90-1.80 (m, |
| | 1H), 1.59 (s, 3H), 1.10-0.92 (m, 4H). |
| 263 | 1H-NMR: (400 MHz, DMSO-d6): δ: 8.59 (s, 1H), 7.58 (dd, J = 2.8 Hz, 1H), |
| | 7.43 (d, J = 2.8 Hz, 1H), 7.34-7.30 (m, 2H), 4.31-4.30 (m, 2H), 4.07-4.05 |
| | (m, 2H), 3.32-3.30 (m, 1H), 2.88-2.60 (m, 9H), 2.47 (s, 3H), 2.20-2.15 (m, |
| | 3H), 1.94-1.90 (m, 2H), 1.61 (s, 3H), 0.48-0.46 (m, 2H), 0.29 (m, 2H) |
| 267 | 1H-NMR (400 MHz, DMSO-d6): δ 8.71 (s, 1H), 7.57 (dd, J = 2.80 , 8.80 Hz, |
| | 1H), 7.44 (d, J = 2.80 Hz, 1H), 7.39 (s, 1H), 7.32 (d, J = 9.20 Hz, 1H), 4.32 (t, |
| | J = 4.80 Hz, 2H), 4.07 (t, J = 4.80 Hz, 2H), 2.72 (s, 3H), 2.67-2.66 (m, 2H), 2.50- |
| | 2.46 (m, 3H), 2.29 (s, 2H), 1.62 (s, 3H), 1.61-1.54 (m, 4H), 1.29-1.26 (m, 4H), |
| | 0.40-0.36 (m, 2H), 0.27- 0.25 (m, 2H). |
| 268 | 1H-NMR (400 MHz, DMSO-d6): 8.70 (s, 1H), 7.57 (dd, J = 2.40 , 8.80 Hz, |
| | 1H), 7.45 (d, J = 2.40 Hz, 1H), 7.39 (s, 1H), 7.32 (d, J = 9.20 Hz, 1H), 4.31 (t, |
| | J = 4.80 Hz, 2H), 4.08 (t, J = 4.80 Hz, 2H), 2.73 (s, 3H), 2.34 (t, J = 5.60 Hz, |
| | 4H), 2.23-2.18 (m, 2H), 2.18 (s, 3H), 2.13 (s, 2H), 1.65 (s, 3H), 1.54 (t, J = 6.40 |
| | Hz, 2H ), 1.35-1.32 (m, 4H). |
| 269 | 1H-NMR (400 MHz, DMSO-d6): δ 8.53 (s, 1H), 8.02 (s, 1H), 7.52 (dd, J = |
| | 2.40, 8.80 Hz, 1H), 7.37(d, J = 2.40 Hz, 1H), 7.27 (s, 1H), 4.27 (t, J = 4.80 Hz, |
| | 2H), 4.07 (t, J = 4.80 Hz, 2H), 2.95 (d, J = 6.8 Hz, 2H) , 2.85 (d , J = 6.8 Hz, |
| | 2H) , 2.67 (s, 3H), 2.49 (s, 4H), 2.31 (s, 3H), 1.84-1.82 (m, 2H), 1.67 (s, 3H). |
| 270 | 1H-NMR (400 MHz, DMSO-d6): δ 13.17 (bs, 1H), 8.63 (s, 1H), 7.57 (dd, J = |
| | 2.80, 8.80 Hz, 1H), 7.44 (d, J = 2.80 Hz, 1H), 7.38 (s, 1H), 7.30 (d, J = 9.20 |
| | Hz, 1H), 4.32 (t, J = 4.80 Hz, 2H), 4.07 (t, J = 4.80 Hz, 2H), 3.04 (d, J = 6.8 |
| | Hz, 2H), 2.96 (d, J = 8.00 Hz, 2H), 2.71 (s, 3H), 2.51 (s, 2H), 2.45 (t, J = 1.60 |
| | Hz, 2H), 1.89-1.88 (m, 1H), 1.85 .- 1.80 (m, 2H ), 1.58 (s, 3H), 0.31-0.27 (m, |
| | 2H), 0.2-0.18 (m, 2H) |
| 360 | 1H-NMR: (400 MHz, DMSO-d6): δ 13.44 (br s, 1H), 8.75 (br s, 1H), 7.57 |
| | (dd, J = 4 Hz, J = 8 Hz, 1H), 7.43 (d, J = 4 Hz, 1H), 7.39 (s, 1H), 7.33 (d, J = 8 |
| | Hz, 1H), 6.71 (t, 1H), 4.32 (t, J = 4 Hz, 2H), 4.09 (t, J = 4 Hz, 2H), 2.88-2.72 |
| | (m, 4H), 2.58-2.55 (m, 1H), 2.71 (s, 3H), 2.46-2.44 (m, 3H), 2.40-2.30 (m, 1H), |
| | 2.22 2.15 (m, 1H), 1.90 (s, 3H), 1.60-1.56 (m, 6H). |
| 361 | 1H-NMR: (400 MHz, DMSO-d6): 8.73 (s, 1H), 7.58 (dd, J = 2.80, 9.00 Hz, 1H), |
| | 7.44 (d, J = 2.80 Hz, 1H), 7.40 (s, 1H), 7.34 (s, 1H), 4.34-4.31 (m, 2H), 4.08- |
| | 4.06 (m, 2H), 3.24 (m, 1H), 3.11 (m, 1H), 2.85-2.84 (m, 2H), 2.76 (m, 2H), 2.67 |
| | (s, 3H), 2.51 (m, 2H), 2.49-2.48 (m, 2H), 2.29-2.27 (m, 1H), 1.92-1.90 (m, 2H), |
| | 1.66-164 (m, 2H), 1.60 (s, 3H) ppm |
| 362 | 1H-NMR: (400 MHz, DMSO-d6): δ 8.46 (s, 1H), 7.55 (dd, J = 2.40, 8.80 Hz, |
| | 1H), 7.41 (d, J = 2.40 Hz, 1H), 7.29 (t, J = 7.60 Hz, 2H), 4.42-4.44 (m, 1H), |
| | 4.30 (m, 2H), 4.07 (m, 2H), 2.52-2.74 (m, 7H), 2.40-2.49 (m, 3H), 2.07-2.20 |
| | (m, 1H), 1.85-1.93 (m, 3H), 1.54-1.63 (s, 6H) |
| 363 | 1H-NMR: (400 MHz, DMSO-d6): δ: 12.76 (br s, 1H), 8.89 (br s, 1H), 7.59 |
| | (dd, J = 4 Hz, J = 8 Hz, 1H), 7.42-7.32 (m, 3H), 4.33 (t, J = 4 Hz, 2H), 4.07 |
| | (t, J = 4 Hz, 2H), 3.39 (s, 3H), 3.16 (t, J = 12 Hz, 2H), 3.07 (d, J = 8 Hz, 2H), |
| | 2.71 (s, 3H), 2.45-2.27 (m, 5H), 1.99-1.90 (m, 1H), 1.85-1.63 (m, 8 H), 1.63 (s, 3H) |
| 364 | 1H-NMR: (400 MHz, DMSO-d6): δ: 12.76 (br s, 1H), 8.89 (br s, 1H), 7.57 |
| | (dd, J = 4 Hz, J = 8 Hz, 1H), 7.42-7.31 (m, 3H), 4.33 (t, J = 4 Hz, 2H), 4.07 |
| | (t, J = 4 Hz, 2H), 3.33 (s, 3H), 3.17-3.05 (m, 4H), 2.71 (s, 3H), 2.45-2.27 (m, |
| | 5H), 1.99-1.90 (m, 1H), 1.85-1.63 (m, 11 H), |
| 365 | 1H NMR (400 MHz, DMSO-d6): δ 8.88 (s, 1H), 8.83-8.80 (m, 1H), 7.74- |
| | 7.67 (m, 3H), 7.51 (dd, J = 1.20, 4.60 Hz, 1H), 4.75 (s, 2H), 3.60- |
| | 3.49 (m, 2H), 3.47-3.46 (m, 2H), 3.36-3.30 (m, 4H), 3.12-3.10 (m, 1H), 2.81- |
| | 2.77 (m, 1H), 2.67-2.66 (m, 2H), 2.50-2.49 (m, 1H), 2.25-2.22 (m, 2H), 2.10- |
| | 2.07 (m, 1H), 1.96-1.90 (m, 3H), 1.88-1.83 (m, 2H), 1.62-1.58 (m, 1H). |
| 366 | 1H-NMR: (400 MHz, DMSO-d6): δ: 8.35 (s, 1H), 8.83 (s, 1H), 7.58 (dd, J = 2.4 |
| | Hz, 8.8 Hz, 1H), 7.43 (d, J = 2.4 Hz, 1H), 7.36 (s, 1H), 7.33 (s, 1H), 4.83- |
| | 4.71 (m, 1H), 4.32 (t, J = 4.8 Hz, 2H), 4.08 (t, J = 4.4 Hz, 2H ), 3.38-3.29 (m, |
| | 4H), 3.00-2.99 (m, 1H), 2.72-2.68 (m, 3H), 2.66-2.57 (m, 2H), 2.50-2.49 (m, |
| | 4H), 2.20-2.13 (m, 1H), 1.89-1.86 (m, 1H), 1.75-1.59 (m, 6H). |
| 367 | 1H-NMR: (400 MHz, DMSO-d6): δ: 8.50 (s, 1H), 7.57 (dd, J = 2.8 Hz, 9.2 |
| | Hz, 1H), 7.40 (d, J = 4.0 Hz, 1H), 7.31 (s, 1H), 4.83-4.71 (m, 1H), 4.31- |
| | 4.29 (m, 2H), 4.08 (d, J = 4.0 Hz, 2H), 3.32-3.29 (m, 4H), 3.13- |
| | 2.94 (m, 1H), 2.76-2.69 (m, 1H), 2.67-2.66 (m, 3H), 2.66-2.62 (m, 2H), 2.50- |
| | 2.49 (m, 2H), 2.33-2.32 (m, 1H), 2.29-2.22 (m, 1H), 1.85-1.82 (m, 1H), 1.76- |
| | 1.60 (m, 6H), 1.09-1.07 (m, 2H). |
| 368 | 1H NMR: (400 MHz, DMSO-d6): δ 8.70 (s, 1H), 7.56 (dd, J = 2.80, 9.00 Hz, |
| | 1H), 7.42 (d, J = 2.80 Hz, 1H), 7.34 (d, J = 9.60 Hz, 2H), 4.31 (t, J = 4.80 Hz, |
| | 2H), 4.07 (t, J = 5.20 Hz, 1H), 2.67-2.95 (m, 3H), 2.51 (s, 3H), 2.49-2.51 (m, |
| | 3H), 2.25-2.41 (m, 4H), 2.40 (t, J = 7.60 Hz, 3H), 2.21-2.27 (m, 3H), 1.91-1.93 |
| | (m, 3H), 1.62 (s, 3H), 1.58-1.59 (m, 1H) |
| 369 | 1H NMR: (400 MHz, DMSO-d6): δ 8.74 (s, 1H), 7.56 (dd, J = 2.40, 8.80 Hz, |
| | 1H), 7.42 (d, J = 2.40 Hz, 1H), 7.31 (d, J = 9.20 Hz, 2H), 4.24 (t, J = 88.40 Hz, |
| | 2H), 4.07 (t, J = 4.80 Hz, 2H), 3.15-3.40 (m, 3H), 2.70 (s, 3H), 2.59-2.62 (m, |
| | 3H), 2.52-2.58 (m, 4H), 2.32-2.48 (m, 3H), 2.18-2.89 (m, 3H), 1.72-1.99 (m, |
| | 3H), 1.62 (s, 4H) ppm. |
| 370 | 1H-NMR: (400 MHz, DMSO-d6): δ 8.45 (s, 1H), 7.55 (dd, J = 2.80, 8.80 Hz, |
| | 1H), 7.41 (d, J = 2.40 Hz, 1H), 7.29 (t, J = 11.20 Hz, 2H), 4.40-4.44 (m, 1H), |
| | 4.30 (m, 2H), 4.08 (m, 2H), 2.67-2.80 (m, 5H), 2.57-2.60 (m, 2H), 2.37-2.44 |
| | (m, 3H), 2.32-2.33 (m, 1H), 1.85-1.92 (m, 3H), 1.54-1.66 (m, 6H) |
| 371 | 1H-NMR: (400 MHz, DMSO-d6): δ 7.90 (brs, 1H), 7.51 (dd, J = 4.0, 12.0 Hz, |
| | 1H), 7.38 (d, J = 4.0 Hz, 1H), 7.24 (d, J = 8.0 Hz, 1H), 7.02 (s, 1H), 4.25 (t, J = |
| | 8.0 Hz, 2H), 4.06 (t, J = 8.0 Hz, 2H), 3.21 (s, 3H), 3.20-3.10 (m, 1H), 2.80- |
| | 2.60 (m, 6H), 2.30-2.20 (m, 2H), 1.90 (t, J = 1.2 Hz, 1H), 1.82 (s, 3H), 1.65 (s, |
| | 3H), 1.46-1.33 (m, 4H), 0.85-0.80 (m, 2H ), 0.72-0.71(m, 2H). |
| 372 | 1H-NMR: (400 MHz, DMSO-d6): δ 8.13 (brs, 1H), 7.52 (dd, J = 4.0, 12.0 Hz, |
| | 1H), 7.39 (s, 1H), 7.28 (d, J = 12.0 Hz, 1H), 7.08-7.07 (m, 1H), 4.26 (s, |
| | 2H), 4.06 (s, 2H), 3.22 (s, 3H), 3.20-3.10 (m, 1H), 2.80-2.60 (m, 6H), 2.30- |
| | 2.20 (m, 2H), 1.90 (t, J = 12.0 Hz, 1H), 1.82 (s, 3H), 1.65 (s, 3H), 1.46-1.33 |
| | (m, 4H), 1.26 (s, 2H), 0.85-0.80 (m, 2H). |
| 373 | 1H NMR (400 MHz, DMSO-d6): δ 12.999 (brs, 1H), 8.66 (s, 1H), 7.57 (dd, J = |
| | 2.80, 9.00 Hz, 1H), 7.43 (d, J = 2.40 Hz, 1H), 7.33 (t, J = 9.20 Hz, 2H), 4.32- |
| | 4.30 (m, 2H), 4.08-4.01 (m, 2H), 3.01-2.93 (m, 2H), 2.72 (s, 3H), 2.51-2.49 (m, |
| | 2H), 2.27-2.17 (m, 4H), 1.90-1.82 (m, 1H), 1.82-1.79 (m, 3H), 1.60 (s, 3H), |
| | 1.46-1.40 (m, 2H), 1.39-1.33 (m, 2H). |
| 374 | 1H NMR (400 MHz, DMSO-d6): δ 8.82 (s, 1H), 7.58 (dd, J = 2.80, 9.00 Hz, |
| | 1H), 7.44 (t, J = 3.20 Hz, 2H), 7.33 (d, J = 8.80 Hz, 1H), 4.33-4.32 (m, 2H), |
| | 4.07-.4.05 (m, 2H), 3.05-2.94 (m, 2H), 2.74 (s, 3H), 2.68-2.51 (m, 1H), 2.51- |
| | 2.50 (m, 2H), 2.34-2.18 (m, 4H), 1.91-1.79 (m, 3H), 1.63-1.61 (m, 1H), 1.58- |
| | 1.56 (m, 1H), 1.49-1.40 (m, 2H), 1.35 (s, 1H), 1.29-1.25 (m, 2H) |
| 375 | 1H-NMR: (400 MHz, DMSO-d6): 8.73 (s, 1H), 7.58 (dd, J = 2.80, 9.00 Hz, 1H), |
| | 7.44 (d, J = 2.80 Hz, 1H), 7.40 (s, 1H), 7.34 (s, 1H), 4.32-4.31 (m, 2H), 4.07- |
| | 4.09 (m, 2H), 3.22-3.24 (m, 1H), 3.12 (d, J = 9.20 Hz, 1H), 2.89-2.88 (m, 2H), |
| | 2.86-2.85 (m, 2H), 2.73 (m, 3H), 2.51 (m, 2H), 2.50-2.49 (m, 2H), 2.26(m, 1H), |
| | 1.99 (m, 2H), 1.66 (m, 2H), 1.60 (m, 3H) ppm |
| 376 | 1H-NMR: (400 MHz, DMSO-d6): 8.79 (m, 1H), 7.58 (dd, J = 2.80, 8.80 Hz, |
| | 1H), 7.43 (d, J = 6.40 Hz, 2H), 7.34-7.32 (m, 1H), 4.31-4.33 (m, 2H), 4.07-4.09 |
| | (m, 2H), 3.25 (s, 3H), 3.17-3.08 (m, 2H), 2.83-2.72 (m, 1H), 2.73 (s, 3H), 2.51- |
| | 2.45 (m, 4H), 2.23-1.81 (m, 5H), 1.58-1.55 (m, 1H), 1.57 (s, 3H), 1.40-1.38 (m, |
| | 1H), 1.12-0.88 (m, 6H), |
| 377 | 1H-NMR: (400 MHz, DMSO-d6): 8.79 (s, 1H), 7.58 (dd, J = 2.40, 9.00 Hz , 1H), |
| | 7.44 (d, J = 4 Hz, 2H), 7.34-7.32 (m, 1H), 4.33-4.31 (m, 2H), 4.09-4.07 (m, |
| | 2H), 3.53 (m, 1H), 3.32 (s, 3H), 3.17-3.08 (m, 2H), 2.86-2.82 (m, 2H), 2.74 (s, |
| | 3H), 2.56-2.44 (m, 4H), 2.20-1.81 (m, 4H), 1.58-1.55 (m, 1H), 1.56 (s, 3H), |
| | 1.24-0.99 (m, 6H), |
| 378 | 1H-NMR: (400 MHz, DMSO-d6): 8.71 (s, 1H), 7.57 (dd, J = 2.40, 9.00 Hz, 3H), |
| | 7.43-7.44 (m, 1H), 7.39 (s, 1H), 7.32 (dd, J = 7.32 Hz, 1H), 4.31-4.30 (m, 2H), |
| | 4.08-4.05 (m, 2H), 3.32-3.25- (m, 3H), 2.74-2.70 (m, 2H), 2.65 (s, 3H), 2.51- |
| | 2.46 (m, 1H), 2.44-2.39 (m, 4H), 2.33-2.28(m, 1H), 1.94-1.82-(m, 2H), 1.59 (s, |
| | 3H), 1.54-1.52 (m, 1H), 1.40-1.37- (m, 1H), 0.89-0.90 (m, 6H), |
| 379 | 1H-NMR: (400 MHz, DMSO-d6): δ 8.79 (brs, 1H), 7.52 (dd, J = 4.0, 8.0 Hz, 1H), |
| | 7.44 (d, J = 4.0 Hz, 1H ), 7.39 (s, 1H), 7.34 (d, J = 8.0 Hz, 1H ), 4.45-4.27 (m, |
| | 3H), 4.1-4.05 (m, 2H), 2.81-2.71 (m, 6H), 2.65-2.55 (m, 1H), 2.45-2.35 |
| | (m, 2H), 2.27-2.20 (m, 1H), 1.95-1.86 (m, 3H), 1.76-1.71 (m, 5H), 1.33- |
| | 1.30 (m, 2H), 0.77-0.75 (m, 1H), 0.68-0.67 (m, 2H). |
| 380 | 1H-NMR: (400 MHz, DMSO-d6): δ 8.74 (brs, 1H), 7.52 (dd, J = 4.0, 8.0 Hz, 1H), |
| | 7.44 (d, J = 4.0 Hz, 1H ), 7.35 (s, 1H), 7.33 (d, J = 8.0 Hz, 1H ), 4.41 (t, J = 4.0 |
| | Hz, 1H), 4.36 (t, J = 8.0 Hz, 2H), 4.06 (t, J = 8.0 Hz, 2H), 2.80-2.70 (m, 6H), |
| | 2.65-2.55 (m, 1H), 2.45-2.40 (m, 2H), 2.27-2.20 (m, 1H), 1.95-1.86 (m, |
| | 3H), 1.76-1.71 (m, 5H), 1.33-1.30 (m, 2H), 0.77-0.75 (m, 1H), 0.68-0.67 |
| | (m, 2H). |
| 381 | 1H-NMR: (400 MHz, DMSO-d6): 8.71 (s, 1H), 7.57 (dd, J = 2.40, 9.00 Hz, 1H), |
| | 7.43 (d, J = 2.40 Hz, 1H), 7.39 (s, 1H), 7.32 (dd, J = 9.20, Hz, 1H), 4.32 (m, |
| | 2H), 4.06-4.08 (m, 2H), 3.08 (d, J = 10.00 Hz, 1H), 2.91 (d, J = 11.20 Hz, 1H), |
| | 2.72-2.68 (m, 4H), 2.49-2.51 (m, 4H), 2.16-2.24 (m, 3H), 1.87 (d, J = 11.60 |
| | Hz, 2H), 1.74-1.71 (m, 1H), 1.60 (m, 4H), 1.53-1.58 (m, 1H), 1.26-1.34 (m, 1H) |
| 382 | 1H-NMR: (400 MHz, DMSO-d6): 8.59 (m, 1H), 7.56 (dd, J = 2.40, 9.00 Hz, |
| | 1H), 7.43-7.42 (m, 1H), 7.43 (d, J = 2.40 Hz, 1H), 7.33-7.32 (m, 1H), 4.31 (m, |
| | 2H), 4.08-4.06 (m, 2H), 3.01 (d, J = 10.40 Hz, 1H), 2.91 (d, J = 11.20 Hz, 1H), |
| | 2.68-2.67 (m, 4H), 2.51-2.49 (m, 4H), 2.26-2.12 (m, 3H), 1.88-1.86 (m, 2H), |
| | 1.74-1.71 (m, 1H), 1.60 (m, 4H), 1.58-1.46 (m, 1H), 1.27-1.24 (m, 1H) |
| 383 | 1H-NMR: (400 MHz, DMSO-d6): 8.507 (s, 1H), 7.55-7.58 (m, 1H), 7.39-7.37 |
| | (d, J = 2.4 Hz, 1H), 7.28-7.30 (d, J = 8.8 Hz, 2H), 4.31 (m, 2H), 4.09 (m, 2H), |
| | 3.02 (d, J = 9.20 Hz, 1H), 2.92 (d, J = 10.80 Hz, 1H), 2.63 (m, 4H), 2.47-2.53 |
| | (m, 4H), 2.12-2.26 (m, 3H), 1.87-1.93 (m, 2H), 1.74 (d, J = 12.80 Hz, 1H), 1.60- |
| | 1.58 (m, 4H), 1.45-1.56 (m, 1H), 1.23-1.34 (m, 1H), |
| 384 | 1H-NMR: (400 MHz, DMSO-d6): δ 8.701 (s, 1H), 7.58-7.58 (dd, J = 3.86 Hz, |
| | 1H), 7.43-7.42 (d, J = 2.8 Hz, 1H), 7.38 (s, 1H), 7.33-7.31 (d, J = 8.8 Hz, 1H), |
| | 4.31-4.32 (m, 2H), 4.06-4.08 (m, 2H), 3.08 (d, J = 9.20 Hz, 1H), 2.84 (d, J = |
| | 11.20 Hz, 1H), 2.67-2.72 (m, 4H), 2.45-2.50 (m, 4H), 2.16-2.24 (m, 3H), 1.87 |
| | (d, J = 12.40 Hz, 2H), 1.71-1.75 (m, 1H), 1.58-1.63 (m, 4H), 1.46-1.40 (m, 1H), |
| | 1.35-1.32 (m, 1H), |
| 385 | 1H-NMR: (400 MHz, DMSO-d6): δ 8.77 (br s, 1H), 7.57 (dd, J = 4 Hz, J = 8 Hz, |
| | 1H), 7.43 (d, J = 4 Hz, 1H), 7.39 (s, 1H), 7.33 (d, J = 8 Hz, 1H), 6.71 (t, 1H), |
| | 4.32 (t, J = 4 Hz, 2H), 4.09 (t, J = 4 Hz, 2H), 2.88-2.72 (m, 4H), 2.58-2.55 (m, |
| | 1H), 2.71 (s, 3H), 2.46-2.44 (m, 3H), 2.40-2.30 (m, 1H), 2.22 2.15 (m, 1H), |
| | 1.90 (s, 3H), 1.60-1.56 (m, 6H). |
| 386 | 1H-NMR: (400 MHz, DMSO-d6): δ 13.39 (s, 1H), 8.62 (s, 1H), 7.56 (dd, J = |
| | 2.40, 9.00 Hz, 1H), 7.42 (d, J = 2.40 Hz, 1H), 7.35 (d, J = 6.40 Hz, 1H), 7.30 (s, |
| | 1H), 4.94 (s, 1H), 4.32 (t, J = 5.20 Hz, 2H), 4.07 (t, J = 4.80 Hz, 2H), 2.91 (d, |
| | J = 10.40 Hz, 1H), 2.83-2.77 (m, 2H), 2.70 (s, 3H), 2.55 (m, 1H), 2.46-2.41 (m, |
| | 4H), 2.27-2.21(m, 2H), 1.95-1.84 (m, 2H), 1.64-1.59 (m, 4H), 1.23 (s, 1H). |
| 387 | 1H-NMR: (400 MHz, DMSO-d6): δ 13.39 (s, 1H), 8.70 (s, 1H), 7.57 (dd, J = |
| | 2.40, 9.00 Hz, 1H), 7.43 (d, J = 2.80 Hz, 1H), 7.38 (s, 1H), 7.32 (d, J = 9.20 Hz, |
| | 1H), 4.96 (s, 1H), 4.33 (t, J = 4.40 Hz, 2H), 4.06 (t, J = 5.20 Hz, 2H), 2.95 (d, |
| | J = 5.60 Hz, 1H), 2.84-2.86 (m, 2H), 2.72 (s, 3H), 2.51 (m, 1H), 2.39-2.44 (m, |
| | 4H), 2.23-2.29 (m, 2H), 1.91-1.90 (m, 2H), 1.67-1.57 (m, 4H), 1.23 (s, 1H). |
| 388 | 1H-NMR: (400 MHz, DMSO-d6): δ 12.01 (s, 1H), 8.57 (s, 1H), 7.56 (dd, J = |
| | 2.40, 9.00 Hz, 1H), 7.42 (d, J = 2.40 Hz, 1H), 7.33 (d, J = 2.80 Hz, 1H), 7.30 (s, |
| | 1H), 4.95 (s, 1H), 4.32 (t, J = 4.80 Hz, 2H), 4.07 (t, J = 4.80 Hz, 2H), 2.90-2.92 |
| | (m, 1H), 2.77-2.81 (m, 2H), 2.70 (s, 3H), 2.62-2.67 (m, 1H), 2.51-2.55 (m, 1H), |
| | 2.41-2.46 (m, 3H), 2.21-2.28 (m, 2H), 1.85-1.95 (m, 2H), 1.61-1.64 (m, 3H), |
| | 1.24 (s, 1H). |
| 389 | 1H-NMR: (400 MHz, DMSO-d6): δ 13.16 (s, 1H), 8.74 (s, 1H), 7.57 (dd, J = |
| | 2.80, 8.80 Hz, 1H), 7.43 (d, J = 2.80 Hz, 1H), 7.40 (s, 1H), 7.33 (d, J = 8.80 Hz, |
| | 1H), 4.98-4.96 (m, 1H), 4.34 (t, J = 4.80 Hz, 2H), 4.06 (t, J = 6.40 Hz, 2H), |
| | 2.96-2.93 (m, 1H), 2.74-2.86 (m, 2H), 2.73 (s, 3H), 2.52-2.51 (m, 1H), 2. 44- |
| | 2.38 (m, 3H), 2.32-2.24 (m, 2H), 1.90 (m, 2H), 1.68-1.65(m, 1H), 1.57 (s, 3H), |
| | 1.23 (s, 1H). |
| 390 | 1H-NMR: (400 MHz, DMSO-d6): δ 13.65 (s, 1H), 8.82 (s, 1H), 7.58 (dd, J = |
| | 2.80, 8.80 Hz, 1H), 7.45-7.43 (m, 2H), 7.33 (d, J = 9.20 Hz, 1H), 4.35-4.31 (m, |
| | 2H), 4.07 (t, J = 5.20 Hz, 2H), 2.74-2.58 (m, 8H), 2.49-2.47 (m, 3H), 2.27-2.24 |
| | (m, 1H), 1.91-1.89 (m, 1H), 1.63-1.58 (m, 8H), 1.24 (m, 2H). |
| 391 | (1H-NMR: (400 MHz, DMSO-d6): 13.73 (s, 1H), 8.79 (s, 1H), 7.58 (dd, J = |
| | 2.80, 8.80 Hz, 1H), 7.42-7.44 (m, 2H), 7.33 (d, J = 8.80 Hz, 1H), 4.33-4.32 (m, |
| | 2H), 4.08 (d, J = 4.40 Hz, 2H), 2.73 (s, 3H), 2.67-2.66 (m, 8H), 2.50 (s, 3H), |
| | 2.25-2.18 (m, 1H), 1.89 (d, J = 11.60 Hz, 1H), 1.67-1.60 (m, 7H). |
| 392 | 1H-NMR: (400 MHz, DMSO-d6): 8.63 (s, 1H), 7.57 (dd, J = 2.80, 8.80 Hz, 1H), |
| | 7.43 (d, J = 2.40 Hz, 1H), 7.36 (s, 1H), 7.33 (s, 1H), 7.31 (s, 1H), 4.31-4.30 (m, |
| | 2H), 4.01-4.04 (m, 2H), 2.78-2.76 (m, 1H), 2.72 (s, 3H), 2.86-2.85 (m, 2H), |
| | 2.58-2.51 (m, 2H), 2.50-2.48 (m, 4H), 2.17-2.09 (m, 2H), 1.89-1.80 (m, 3H), |
| | 1.6 (s, 3H), 1.54-1.50 (m, 1H), 0.96 (s, 3H), 0.95 (s, 3H). |
| 393 | 1H-NMR: (400 MHz, DMSO-d6): 8.48 (s, 1H), 7.55 (dd, J = 2.80, 8.80 Hz, 1H), |
| | 7.41 (d, J = 2.40 Hz, 1H), 7.30 (d, J = 9.20 Hz, 2H), 4.30 (s, 2H), 4.02-4.06 (m, |
| | 3H), 2.82-2.80 (m, 1H), 2.59 (m, 3H), 2.55-2.54 (m, 2H), 2.50-2.49 (m, 4H), |
| | 2.09 (m, 2H), 1.90 (m, 3H), 1.62 (m, 4H), 1.24 (s, 1H), 0.95 (s, 6H). |
| 394 | 1H-NMR: (400 MHz, DMSO-d6): 8.63 (s, 1H), 7.57 (dd, J = 2.40, 9.00 Hz, 1H), |
| | 7.43 (d, J = 2.40 Hz, 1H), 7.33 (t, J = 9.20 Hz, 2H), 4.31 (s, 2H), 4.07- 4.02 (m, |
| | 3H), 2.85-2.83 (m, 1H), 2.71 (s, 3H), 2.55-2.51 (m, 2H), 2.50-2.49 (m, 4H), |
| | 2.18-2.08 (m, 2H), 1.85-1.79 (m, 3H), 1.60-1.54 (m, 4H), 0.96 (s, 6H) |
| 395 | 1H-NMR: (400 MHz, DMSO-d6): 8.41 (s, 1H), 7.55 (dd, J = 2.80, 8.80 Hz, 1H), |
| | 7.41 (d, J = 2.80 Hz, 1H), 7.31-7.26(m, 2H), 4.30 (t, J = 4.80 Hz, 2H), 4.06-4.01 |
| | (m, 2H), 2.78-2.75 (m, 1H), 2.69-2.58 (m, 3H), 2.51-2.50 (m, 2H), 2.50-2.49 |
| | (m, 4H), 2.17-2.12 (m, 2H), 1.89-1.88 (m, 3H), 1.63 (s, 3H), 1.54-1.53 (m, 1H), |
| | 1.23-1.22 (m, 1H), 0.97 (s, 3H), 0.96 (s, 3H). |
| 396 | 1H-NMR: (400 MHz, DMSO-d6): δ 8.49 (s, 1H), 7.56 (dd, J = 2.80, 8.80 Hz, |
| | 1H), 7.42 (d, J = 2.80 Hz, 1H), 7.31 (d, J = 8.80 Hz, 2H), 4.64 (t, J = 4.40 Hz, |
| | 1H), 4.30-4.33 (m, 2H), 4.07-4.09 (m, 2H), 3.46 (d, J = 16.00 Hz, 2H), 2.70 (s, |
| | 3H), 2.650(m, 1H), 2.56 (d, J = 4.40 Hz, 1H), 2.49-2.39 (m, 2H), 2.18-2.08 (m, |
| | 3H), 1.95-1.92 (m, 1H), 1.90-1.84 (m, 4H), 1.65 (m, 2H), 1.63 (s, 3H), 1.54-1.47 |
| | (m, 1H) |
| 397 | 1H-NMR: (400 MHz, DMSO-d6): δ 8.71 (s, 1H), 7.57 (dd, J = 2.80, 8.80 Hz, |
| | 1H), 7.44 (d, J = 2.80 Hz, 1H), 7.40 (s, 1H), 7.32 (d, J = 8.80 Hz, 1H), 4.66-4.58 |
| | (m, 1H), 4.34 (t, J = 5.20 Hz, 2H), 4.07 (t, J = 4.80 Hz, 2H), 3.60 (s, 1H), 3.52 |
| | (s, 1H), 2.84 (s, 1H), 2.73 (s, 3H), 2.55-2.54 (m, 1H), 2.45-2.40 (m, 2H), 2.16 |
| | (dd, J = 9.20, Hz, 1H), 1.97-1.94 (m, 1H), 1.81-1.75 (m, 6H), 1.63 (s, 3H), |
| | 1.60-1.53 (m, 3H) |
| 398 | 1H-NMR: (400 MHz, DMSO-d6): δ 8.70 (s, 1H), 7.57 (dd, J = 2.80, 8.80 Hz, |
| | 1H), 7.44 (d, J = 2.80 Hz, 1H), 7.40 (s, 1H), 7.33 (d, J = 9.20 Hz, 1H), 4.65 (t, |
| | J = 5.20 Hz, 1H), 4.33 (t, J = 2.00 Hz, 2H), 4.08 (t, J = 4.40 Hz, 2H), 3.46 (d, J = |
| | 17.60 Hz, 2H), 2.72 (s, 3H), 2.56-2.55 (m, 2H), 2.45 (m, 1H), 2.39-2.37 (m, |
| | 1H), 2.19-2.07 (m, 3H), 1.95-1.84 (m, 5H), 1.679 (s, 3H), 1.64-1.50 (m, 3H) |
| 399 | 1H-NMR: (400 MHz, DMSO-d6): δ 8.50 (s, 1H), 7.56 (dd, J = 2.40, 9.00 Hz, |
| | 1H), 7.42 (d, J = 2.40 Hz, 1H), 7.31 (t, J = 4.40 Hz, 2H), 4.57-4.66 (m, 1H), |
| | 4.32 (t, J = 4.80 Hz, 2H), 4.08 (t, J = 4.80 Hz, 2H), 3.55 (d, J = 30.40 Hz, 2H), |
| | 2.84-3.32 (m, 1H), 2.70 (s, 3H), 2.62-2.63 (m, 1H), 2.44-2.50 (m, 2H), 2.15 (dd, |
| | J = 9.20, Hz, 1H), 2.50-2.44 (m, 1H), 1.81-1.78 (m, 6H), 1.618 (s , 3H) , 1.617- |
| | 1.52 (m, 3H) |
| 400 | 1H-NMR: (400 MHz, DMSO-d6):: 8.45 (s, 1H), 8.34 (s, 1H), 7.56 (dd, J = 2.40, |
| | 9.00 Hz, 1H), 7.39 (d, J = 2.40 Hz, 1H), 7.32 (d, J = 8.80 Hz, 1H), 7.22 (s, 1H), |
| | 4.28-4.33 (m, 2H), 4.13 (m, 3H), 3.98 (d, J = 9.60 Hz, 1H), 3.88 (d, J = 10.00 |
| | Hz, 3H), 3.80 (d, J = 9.20 Hz, 2H), 2.76 (d, J = 12.80 Hz, 2H), 2.61-2.71 (m, |
| | 4H), 2.42 (s, 4H), 2.33 (t, J = 2.00 Hz, 2H), 1.87 (1.85-1.90 (m, 4H), 1.38 (s, 3H), |
| 401 | 1H-NMR: (400 MHz, DMSO-d6): δ 13.47 (br s, 1H), 8.79 (s, 1H), 7.58 (dd, J = |
| | 2.40, 8.8 Hz, 1H), 7.42-7.45 (m, 2H), 7.33 (d, J = 8.80 Hz, 1H), 4.30-4.34 (m, |
| | 2H), 4.07-4.08 (m, 2H), 2.76-2.79 (m, 2H), 2.73 (s, 3H), 2.66-2.68 (m, 2H), |
| | 2.51-2.54 (m, 1H), 2.45-2.50 (m, 3H), 2.05-2.15 (m, 5H), 1.78-1.90 (m, 1H), |
| | 1.66-1.69 (m, 2H), 1.49-1.51 (m, 4H). |
| 402 | 1H-NMR: (400 MHz, DMSO-d6): δ 13.58 (br s, 1H), 8.79 (s, 1H), 7.58 (dd, J = |
| | 2.40, 9.00 Hz, 1H), 7.42-7.45 (m, 2H), 7.33 (d, J = 8.80 Hz, 1H), 4.30-4.34 (m, |
| | 2H), 4.07-4.08 (m, 2H), 2.76-2.79 (m, 2H), 2.73 (s, 3H), 2.66-2.68 (m, 2H), |
| | 2.51-2.54 (m, 1H), 2.45-2.50 (m, 3H), 2.05-2.15 (m, 5H), 1.78-1.90 (m, 1H), |
| | 1.66-1.69 (m, 2H), 1.49-1.51 (m, 4H). |
| 403 | 1H-NMR: (400 MHz, DMSO-d6): δ 8.28 (s, 1H), 7.53 (dd, J = 2.80, 9.00 Hz, |
| | 1H), 7.36 (s, 1H), 7.28 (d, J = 8.80 Hz, 1H), 7.16 (s, 1H), 4.82-4.89 (m, 1H), |
| | 4.28 (s, 2H), 4.08 (s, 2H), 3.16 (dd, J = 10.40, 10.00 Hz, 1H), 2.67-2.79 (m, |
| | 4H), 2.58-2.61 (m, 2H), 2.45 (m, 4H), 2.18 (dd, J = 10.40, 15.80 Hz, 1H), 2.03- |
| | 2.07 (m, 1H), 1.827-1.802 (m, 2H), 1.650 (s, 3H), 1.550 (s, 1H) |
| 404 | 1H-NMR: (400 MHz, DMSO-d6): δ 8.33 (s, 1H), 7.53 (dd, J = 2.40, 8.80 Hz, |
| | 1H), 7.37 (s, 1H), 7.28 (d, J = 8.80 Hz, 1H), 7.18 (s, 1H), 4.82-4.89 (m, 1H), |
| | 4.29 (s, 2H), 4.07 (s, 2H), 3.06-3.08 (m, 1H), 2.76-2.85 (m, 4H), 2.56-2.59 (m, |
| | 2H), 2.49-2.51 (m, 4H), 2.14 (dd, J = 18.80, 31.40 Hz, 1H), 2.04-2.02 (m, 1H), |
| | 1.83-1.75 (m, 2H), 1.64 (s, 3H), 1.639-1.587 (m, 1H) |
| 405 | 1H-NMR: (400 MHz, DMSO-d6):: δ 8.46 (s, 1H), 7.55 (dd, J = 2.00, 9.00 Hz, |
| | 1H), 7.40 (s, 1H), 7.27-7.31 (m, 2H), 4.83-4.88 (m, 1H), 4.30 (s, 2H), 4.08 (s, |
| | 2H), 3.06-3.08 (m, 1H), 2.77-2.86 (m, 3H), 2.67-2.69 (m, 3H), 2.57-2.59 (m, |
| | 2H), 2.45-2.46 (m, 2H), 2.16 (dd, J = 11.20, 16.40 Hz, 1H), 2.04-2.07 (m, 1H), |
| | 1.82-1.845 (d, 2H), 1.621 (s, 4H) |
| 406 | 1H-NMR: (400 MHz, DMSO-d6): δ 8.52 (s, 1H), 7.55 (d, J = 8.80 Hz, 1H), 7.41 |
| | (s, 1H), 7.30 (d, J = 8.40 Hz, 2H), 4.83-4.90 (m, 1H), 4.30 (s, 2H), 4.07 (s, 2H), |
| | 3.32 (s, 1H), 2.71-2.80 (m, 6H), 2.57-2.62 (m, 2H), 2.45-2.51 (m, 2H), 2.18 (dd, |
| | J = 10.80, 16.40 Hz, 1H), 2.04-2.07 (m, 1H), 1.81-1.84 (m, 2H), 1.613 (s, 4H) |
| 407 | 1H-NMR: (400 MHz, DMSO-d6): δ 13.65 (s, 1H), 7.57 (dd, J = 2.40, 9.00 Hz, |
| | 1H), 7.43 (d, J = 2.80 Hz, 1H), 7.39 (s, 1H), 7.32 (d, J = 9.20 Hz, 1H), 4.32 (m, |
| | 2H), 4.07-4.08 (m, 2H), 3.92-3.93 (m, 2H), 2.88-2.99 (m, 2H), 2.71 (s, 3H), |
| | 2.51-2.56 (m, 1H), 2.49-2.50 (m, 3H), 2.14-1.91 (m, 4H), 1.59-1.24 (m, 9H), |
| 408 | 1H-NMR: (400 MHz, DMSO-d6): δ 8.74 (s, 1H), 7.58 (dd, J = 2.80, 8.80 Hz, |
| | 1H), 7.44 (d, J = 2.80 Hz, 1H), 7.40 (s, 1H), 7.33 (d, J = 9.20 Hz, 1H), 4.30-4.34 |
| | (m, 2H), 4.07 (d, J = 4.40 Hz, 2H), 3.08-2.71 (m, 3H), 2.73 (s, 3H), 2.51-2.18 |
| | (m, 5H), 1.91-1.86 (m, 1H), 1.71-1.17 (m, 9H), 1.05 (d, J = 6.00 Hz, 2H). |
| 409 | 1H-NMR: (400 MHz, DMSO-d6): δ 8.69 (s, 1H), 7.57 (dd, J = 2.40, 8.80 Hz, |
| | 1H), 7.44 (d, J = 2.80 Hz, 1H), 7.39 (s, 1H), 7.32 (d, J = 9.20 Hz, 1H), 4.28-4.33 |
| | (m, 2H), 4.13-4.02 (m, 2H), 2.95-2.98 (m, 1H), 2.79-2.57 (m, 6H), 2.51-2.13 |
| | (m, 4H), 1.78-1.56 (m, 8H), 1.42-1.20 (m, 3H), 1.00 (d, J = 6.00 Hz, 3H). |
| 410 | 1H NMR (400 MHz, DMSO-d6): δ 8.78 (d, J = 4.8 Hz, 1H), 8.73 (s, 1H), 7.73- |
| | 7.64 (m, 3H), 7.48 (d, J = 4.8 Hz, 1H), 4.73 (s, 2H), 3.23-3.19 (m, 3H), 3.18- |
| | 3.16 (m, 1H), 2.86-2.80 (m, 3H), 2.61-2.53 (m, 2H), 2.50-2.49 (m, 1H), 2.36- |
| | 2.33 (m, 1H), 2.32-2.28 (m, 1H), 2.18-2.14 (m, 1H), 1.93-1.86 (m, 6H), 1.64- |
| | 1.60 (m, 1H), 1.46-1.39 (m, 2H). |
| 411 | 1H-NMR: (400 MHz, DMSO-d6): δ: 8.28 (brs, 1H), 7.56-7.54 (m, 1H), 7.37 (d, |
| | J = 4 Hz, 1H), 7.33 (d, J = 12 Hz, 1H), 7.15 (s, 1H), 6.70 (brs, 2H), 4.27 (t, J = 4 |
| | Hz, 2H), 4.13 (t, J = 4 Hz, 2H), 3.19 (m, 1H), 2.65 (s, 3H), 2.54-2.49 (m, 3H), |
| | 1.93-1.90 (m, 5H), 1.70-1.65 (m, 1H). |
| 412 | 1H-NMR: (400 MHz, DMSO-d6): δ: 8.73 (s, 1H), 7.58 (dd, J = 4.0 Hz, 8.0 |
| | Hz, 1H), 7.44 (d, J = 4.0 Hz, 1H), 7.38 (S, 1H), 7.34 (d, J = 8.0 Hz, |
| | 1H), 4.33 (t, J = 4.0 Hz, 2H), 4.08 (t, J = 4.0 Hz, 2H ), 3.17- |
| | 3.13 (m, 1H), 2.72 (s, 3H), 2.57-2.55 (m, 1H), 2.51-2.46 (m, 2H), 2.04 (s, 4H), |
| | 1.85-1.81 (m, 1H), 1.69-1.57 (m, 16H), 1.24 (s, 1H). |
| 413 | 1H-NMR: (400 MHz, DMSO-d6): δ: 8.67 (s, 1H), 7.57 (dd, J = 4.0 Hz, 10.0 |
| | Hz, 1H), 7.44 (d, J = 4.0 Hz, 1H), 7.36-7.32 (m, 2H), 4.32 (t, J = 4.0 Hz, 2H), |
| | 4.07 (t, J = 4.0 Hz, 2H ), 3.03 (s, 1H), 2.71 (s, 3H), 2.51-2.49 (m, 1H), 2.46- |
| | 2.45 (m, 2H), 2.01 (s, 4H), 1.79-1.77 (m, 1H), 1.65-1.49 (m, 16H), 1.23 (s, 1H). |
| 414 | 1H NMR (400 MHz, DMSO-d6): δ 8.81-8.75 (m, 2H), 7.74-7.66 (m, 3H), |
| | 7.47 (s, 1H), 4.80-4.63 (m, 2H), 3.31-2.96 (m, 12H), 2.60-2.51 (m, 3H), 2.27- |
| | 2.23 (m, 1H), 1.98-1.90 (m, 6H), 1.73-1.56 (m, 3H), 1.24 (s, 1H). |
| 415 | 1H-NMR: (400 MHz, DMSO-d6): 8.79 (s, 1H), 7.57 (dd, J = 2.80, 8.80 Hz, 1H), |
| | 7.45 (d, J = 7.4 Hz, 2H), 7.33-7.31 (m, 1H), 4.33-4.31 (m, 2H), 4.09-4.07 (m, |
| | 2H), 3.25 (s, 3H), 2.83-2.74 (m, 2H), 2. 73 (s, 3H), 2.51-2.45 (m, 5H), 2.23-1.81 |
| | (m, 5H), 1.58-1.55 (m, 1H), 1.57 (s, 3H), 1.40-1.38 (m, 1H), 1.12-0.88 (m, 6H) |
| 416 | 1H-NMR: (400 MHz, DMSO-d6): δ 8.35 (s, 1H), 8.21 (d, J = 7.60 Hz, 1H), 7.47 |
| | (s, 1H), 7.54 (dd, J = 2.80, 8.80 Hz, 1H), 7.30-7.35 (m, 2H), 7.11 (s, 1H), 6.78 |
| | (t, J = 55.20 Hz, 1H), 4.30 (t, J = 6.40 Hz, 2H), 4.05-4.15 (m, 2H), 3.54 (s, 3H), |
| | 3.05-3.23 (m, 2H), 2.57-2.76 (m, 4H). |
| 417 | 1H-NMR: (400 MHz, DMSO-d6): δ 8.94 (s, 1H), 7.59 (dd, J = 2.80, 9.00 Hz, |
| | 1H), 7.47 (s, 1H), 7.44 (d, J = 2.80 Hz, 1H), 7.34 (d, J = 8.80 Hz, 1H), 4.42-4.46 |
| | (m, 1H), 4.32-4.36 (m, 2H), 4.06 (t, J = 4.80 Hz, 2H), 3.22-3.15 (m, 1H), 2.86- |
| | 2.77 (m, 2H), 2.73 (s, 3H), 2.61-2.57 (m, 2H), 2.50-2.44 (m, 4H), 2.22-2.15 (m, |
| | 1H), 1.94-1.86 (m, 3H), 1.70-1.58 (m, 2H), 1.56 (m, 4H), 1.29-1.25 (m, 2H), |
| | 1.24-1.16 (m, 2H), |
| 418 | 1H-NMR: (400 MHz, DMSO-d6): δ 8.76 (s, 1H), 7.58 (dd, J = 2.4, 8.8 Hz, 1H), |
| | 7.44-7.31 (m, 4H), 7.18-7.12 (m, 1H), 7.03-6.98 (m, 1H), 4.36- |
| | 4.32 (m, 2H), 4.15-4.05 (m, 2H), 3.16-3.03 (m, 2H ), 2.71 (s, 3H), 2.65- |
| | 2.62 (m, 2H), 2.36-2.25 (m, 6H), 1.93 (s, 1H), 1.72-1.61 (m, 8H). |
| 419 | 1H-NMR: (400 MHz, DMSO-d6): δ 8.81 (s, 1H), 7.58 (dd, J = 2.4, 8.8 Hz, 1H), |
| | 7.44-7.32 (m, 4H), 7.18-7.12 (m, 1H), 7.03-6.98 (m, 1H), 4.36 (d, J = 10.4 |
| | Hz, 2H), 4.07 (t, J = 4.4 Hz, 2H ), 3.16 (d, J = 26.8 Hz, 1H ), 3.03 (d, J = 11.2 |
| | Hz, 1H ), 2.71 (s, 3H), 2.65-2.62 (m, 2H), 2.36-2.25 (m, 6H), 1.93 (brs, 1H), |
| | 1.72-1.61 (m, 8H). |
| 420 | 1H-NMR: (400 MHz, DMSO-d6): δ 8.70 (s, 1H), 7.57 (dd, J = 2.40, 9.00 Hz, |
| | 1H), 7.43 (d, J = 2.40 Hz, 1H), 7.33 (t, J = 9.20 Hz, 2H), 4.32 (s, 2H), 4.07 (t, |
| | J = 4.40 Hz, 2H), 3.69 (t, J = 6.80 Hz, 2H), 3.17-3.06 (m, 2H), 2.71 (s, 3H), 2.59- |
| | 2.54 (m, 3H), 2.47-2.40 (m, 4H), 2.24-2.20 (m, 1H), 1.87-1.80 (m, 3H), 1.64- |
| | 1.55 (m, 9H), |
| 421 | 1H-NMR: (400 MHz, DMSO-d6): δ 8.30 (s, 1H), 7.54 (dd, J = 2.80, 8.80 Hz, |
| | 1H), 7.39 (d, J = 2.40 Hz, 1H), 7.29 (d, J = 8.80 Hz, 1H), 7.19 (s, 1H), 4.29 (t, |
| | J = 5.20 Hz, 2H), 4.08 (t, J = 4.80 Hz, 2H), 3.69 (t, J = 6.80 Hz, 2H), 3.17 (s, 3H), |
| | 2.63-2.51 (m, 4H), 2.46-2.40 (m, 4H), 2.23-2.19 (m, 1H), 1.88-1.80 (m, 3H), |
| | 1.65-1.53 (m, 10H), |
| 422 | 1H-NMR: (400 MHz, DMSO-d6): δ: 12.93 (brs, 1H), 8.76 (brs, 1H), 8.21 (d, J = |
| | 0.8 Hz, 1H), 7.71(d, J = 0.8 Hz, 1H), 7.57 (dd, J = 2.8 Hz, 8.8 Hz, 1H), 7.44- |
| | 7.40 (m, 2H), 7.34-7.31 (m, 1H), 4.55-4.48 (m, 1H), 4.33 (brs, 2H), 4.07 (brs, |
| | 2H), 3.09-2.99 (m, 2H), 2.73 (s, 3H), 2.68 (m, 1H), 2.52-2.40 (m, 5H), 2.71- |
| | 2.20 (m, 1H), 2.09-1.09 (m, 5H), 1.65-1.60 (m, 5H). |
| 423 | 1H NMR (400 MHz, DMSO-d6): δ: 8.57-8.61 (brs, 1H), 8.21 (d, J = 1.2 Hz, |
| | 1H), 7.71 (d, J = 0.8 Hz, 1H), 7.56 (dd, J = 2.4 Hz, 8.8 Hz, 1H), 7.42 (d, J = 2.4 |
| | Hz, 1H), 7.30-7.32 (m, 2H), 4.54-4.49 (m, 1H), 4.32-4.31 (m, 2H), 4.09-4.08 |
| | (m, 2H), 3.08-2.98 (m, 2H), 2.70 (s, 3H), 2.61-2.59 (m, 1H), 2.51 (s, 3H), 2.43- |
| | 2.40 (m, 2H), 2.27-2.20 (m, 1H), 2.09-2.06 (m, 2H), 2.00-1.89 (m, 4H), 1.62 (s, 3H). |
| 424 | 1H-NMR: (400 MHz, DMSO-d6): 8.81 (s, 1H), 7.58 (dd, J = 2.80, 9.00 Hz, 1H), |
| | 7.42-7.44 (m, 2H), 7.33 (d, J = 8.80 Hz, 1H), 4.31-4.33 (m, 2H), 4.07-4.08 (m, |
| | 2H), 3.61-3.60 (m, 2H), 3.55-3.54 (m, 2H), 3.36-3.33 (m, 2H), 2.73 (s, 3H), |
| | 2.51-2.21 (m, 9H), 1.91-1.77 (m, 3H), 1.63 (m, 1H), 1.58 (s, 3H), 1.51-1.44 (m, 2H). |
| 425 | 1H-NMR: (400 MHz, DMSO-d6): 8.81 (s, 1H), 7.58 (dd, J = 2.80, 9.00 Hz, 1H), |
| | 7.42-7.44 (m, 2H), 7.33 (d, J = 8.80 Hz, 1H), 4.33-4.31 (m, 2H), 4.08-4.07 (m, |
| | 2H), 3.61-3.60 (m, 2H), 3.54-3.53 (m, 2H), 3.36-3.33 (m, 2H), 2.73 (s, 3H), |
| | 2.51-2.25 (m, 9H), 1.77-1.91 (m, 3H), 1.63 (m, 1H), 1.58 (s, 3H), 1.46-1.41 (m, 2H). |
| 426 | 1H-NMR: (400 MHz, DMSO-d6): 8.81 (s, 1H), 7.58 (dd, J = 2.80, 8.80 Hz, |
| | 1H), 7.42-7.45 (m, 3H), 7.33 (d, J = 8.80 Hz, 1H), 7.25 (s, 1H), 4.33-4.33 (m, |
| | 2H), 4.07 (m, 2H), 3.75 (s, 3H), 3.00 (m, 2H), 2.73 (s, 3H), 2.67-2.68 (m, 2H), |
| | 2.38-2.45 (m, 4H), 2.32-2.33 (m, 2H), 1.83-1.91 (m, 3H), 1.61-1.45(m, 6H) |
| 427 | 1H-NMR: (400 MHz, DMSO-d6): 8.81 (s, 1H), 7.58 (dd, J = 2.40, 8.80 Hz, |
| | 1H), 7.42-7.45 (m, 3H), 7.33 (d, J = 9.20 Hz, 1H), 7.25 (s, 1H), 4.32 (m, 2H), |
| | 4.08 (m, 2H), 3.75 (s, 3H), 2.97 (m, 2H), 2.73 (s, 3H), 2.66-2.68 (m, 1H), 2.40- |
| | 2.50 (m, 4H), 2.32-2.38 (m, 3H), 1.87 (m, 3H), 1.64-1.45 (m, 6H) |
| 428 | 1H-NMR: (400 MHz, DMSO-d6): 8.83 (s, 1H), 7.58 (dd, J = 2.80, 8.80 Hz, 1H), |
| | 7.44 (t, J = 2.80 Hz, 2H), 7.33 (d, J = 8.80 Hz, 1H), 7.09 (d, J = 1.20 Hz, 1H), |
| | 4.36-4.32 (m, 2H), 4.07 (s, 2H), 3.05-3.03 (m, 1H), 2.97-2.96 (m, 2H), 2.74 (s, |
| | 3H), 2.67-2.66 (m, 1H), 2.51-2.50 (m, 3H), 2.39 (s, 3H), 2.38-2.36 (m, 3H), 2.16 |
| | (d, J = 3.20 Hz, 2H), 1.91(m, 1H), 1.72 (m, 3H), 1.58 (s, 3H). |
| 429 | 1H-NMR: (400 MHz, DMSO-d6): 8.84 (s, 1H), 7.58 (dd, J = 2.80, 9.00 Hz, 1H), |
| | 7.44-7.45 (m, 2H), 7.33 (d, J = 8.80 Hz, 1H), 7.11 (s, 1H), 4.32-4.37 (m, 2H), |
| | 4.08-4.07 (m, 2H), 3.33-3.30 (m, 3H), 2.74 (s, 3H), 2.67-2.66 (m, 2H), 2.50 (s, |
| | 3H), 2.33-2.32 (s, 3H), 2.32-2.30 (s, 2H), 2.09-2.07 (m, 3H), 1.73-1.70 (m, 3H), |
| | 1.58 (s, 3H), 1.23 (s, 1H). |
| 430 | (1H-NMR: (400 MHz, DMSO-d6): 8.51 (s, 1H), 7.54 (dd, J = 2.40, 8.80 Hz, |
| | 2H), 7.40 (d, J = 2.40 Hz, 1H), 7.30 (d, J = 9.20 Hz, 1H), 7.23 (s, 1H), 4.29 (d, |
| | J = 4.40 Hz, 2H), 4.09 (d, J = 4.40 Hz, 2H), 2.82-2.80 (m, 3H), 2.62-2.60 (m, |
| | 4H), 2.52-2.50 (m, 4H), 2.50 (d, J = 2.00 Hz, 1H), 2.33 (dd, J = 2.00, Hz, 1H), |
| | 1.87-1.82 (m, 3H), 1.65 (s, 6H), |
| 431 | (1H-NMR: (400 MHz, DMSO-d6): 8.50 (s, 1H), 7.56 (dd, J = 2.40, 9.00 Hz, |
| | 2H), 7.42 (d, J = 2.40 Hz, 1H), 7.32 (d, J = 9.20 Hz, 1H), 4.35-4.20 (m, 2H), |
| | 4.15-4.10 (m, 2H), 2.85-2.80 (m, 2H), 2.71 (s, 3H), 2.67-2.66 (m, 3H), 2.50 (s, |
| | 4H), 2.34-2.33 (m, 1H), 2.02-1.80 (m, 3H), 1.62-1.52 (m, 6H), 1.24 (s, 1H), |
| 432 | 1H NMR (400 MHz, DMSO-d6): δ 12.96 (brs, 1H), 8.90 (s, 1H), 7.59 (dd, J = |
| | 4.00, 8.00 Hz, 1H), 7.44 (d, J = 4.00 Hz, 2H), 7.34 (d, J = 12.00 Hz, 1H), 4.33- |
| | 4.30 (s, 2H), 4.08-4.06 (m, 2H), 3.44 (s, 3H), 3.06-2.95 (m, 2H), 2.73 (s, 3H), |
| | 2.67-2.61 (m, 1H), 2.51-2.50 (m, 3H), 2.33-2.32 (m, 4H), 1.91-1.80 (m, 2H), |
| | .61 (s, 3H), 1.47-1.42 (m, 2H), 1.24-1.22 (m, 2H) |
| 433 | 1H NMR (400 MHz, DMSO-d6): δ 8.86 (s, 1H), 7.58 (dd, J = 2.40, 8.80 Hz, |
| | 1H), 7.43 (d, J = 2.40 Hz, 1H), 7.33 (d, J = 8.80 Hz, 2H), 4.32-4.30 (m, 2H), |
| | 4.08 (d, J = 4.40 Hz, 2H), 3.42-3.41 (m, 2H), 3.05-2.95 (m, 2H), 2.68 (s, 3H), |
| | 2.67-2.51 (m, 2H), 2.51-2.49 (m, 4H), 2.33 (t, J = 2.00 Hz, 1H), 2.27-2.22 (m, |
| | 4H), 1.62 (s, 3H), 1.43-1.39 (m, 2H), 1.35-1.23 (m, 2H) |
| 434 | 1H-NMR: (400 MHz, DMSO-d6): δ 8.74 (s, 1H), 7.57 (dd, J = 2.80, 9.00 Hz, |
| | 1H), 7.43 (d, J = 2.40 Hz, 1H), 7.39 (s, 1H), 7.31 (d, J = 0.00 Hz, 1H), 4.52 (s, |
| | 1H), 4.32 (s, 2H), 4.07 (d, J = 4.40 Hz, 2H), 3.44 (s, 1H), 2.78-2.85 (m, 2H), |
| | 2.67 (s, 3H), 2.54 (m, 1H), 2.43-2.46 (m, 3H), 2.20-2.33 (m, 3H), 1.84-1.90 (m, |
| | 1H), 1.72-1.75 (m, 2H), 1.59 (s, 4H), 1.37 (t, J = 9.20 Hz, 2H)ppm |
| 435 | 1H-NMR: (400 MHz, DMSO-d6): δ 8.73 (s, 1H), 7.57 (dd, J = 2.80, 8.80 Hz, |
| | 1H), 7.43 (d, J = 2.80 Hz, 1H), 7.39 (s, 1H), 7.32 (d, J = 8.80 Hz, 1H), 4.52 (s, |
| | 1H), 4.32 (s, 2H), 4.07 (d, J = 4.40 Hz, 2H), 3.45 (m, 1H), 2.72-2.85 (m, 2H), |
| | 2.67 (s, 3H), 2.52-2.56 (m, 1H), 2.46-2.49 (m, 3H), 2.19-2.33 (m, 3H), 8 1.84- |
| | 1.87 (m, 1H), 1.73 (d, J = 10.80 Hz, 2H), 1.59 (s, 4H), 1.37 (t, J = 9.20 Hz, 2H) |
| | ppm |
| 436 | 1H-NMR: (400 MHz, DMSO-d6): δ 8.75 (s, 1H), 7.58 (dd, J = 2.40, 9.00 Hz, |
| | 1H), 7.44 (d, J = 2.80 Hz, 1H), 7.41 (s, 1H), 7.33 (d, J = 8.80 Hz, 1H), 4.30-4.35 |
| | (m, 2H), 4.07-4.08 (m, 2H), 2.86-3.06 (m, 2H), 2.67-2.73 (m, 4H), 2.54-2.22 |
| | (m, 6H), 1.91-1.86 (m, 1H), 1.60-1.24 (m, 10H), 1.05 (d, J = 6.00 Hz, 2H). |
| 437 | 1H-NMR: (400 MHz, DMSO-d6): δ 8.74 (s, 1H), 7.58 (dd, J = 2.40, 9.00 Hz, |
| | 1H), 7.44 (d, J = 2.80 Hz, 1H), 7.40 (s, 1H), 7.33 (d, J = 9.20 Hz, 1H), 4.36-4.28 |
| | (m, 2H), 4.12-4.04 (m, 2H), 2.98-2.50 (m, 6H), 2.50-2.10 (m, 4H), 1.78-1.57 |
| | (m, 8H), 1.42-1.15 (m, 4H), 1.00 (d, J = 6.00 Hz, 3H). |
| 438 | 1H-NMR: (400 MHz, DMSO-d6): δ 8.64 (s, 1H), 7.55 (dd, J = 2.80, 8.80 Hz, |
| | 1H), 7.46 (d, J = 2.80 Hz, 1H), 7.31 (d, J = 2.80 Hz, 1H), 7.33 (s, 1H), 4.30 (t, |
| | J = 5.20 Hz, 2H), 4.08 (t, J = 4.40 Hz, 2H), 2.70 (s, 3H), 2.68-2.67 (m, 2H), 2.59- |
| | 2.54 (m, 2H), 2.49-2.42 (m, 2H), 2.03-2.10 (m, 1H), 1.74 (s, 3H), 1.70-1.62 (m, |
| | 2H), 1.51-1.43 (m, 4H), 1.25-1.11 (m, 8H). |
| 439 | 1H-NMR: (400 MHz, DMSO-d6): δ 8.67 (s, 1H), 7.55 (dd, J = 2.80, 9.00 Hz, |
| | 1H), 7.46 (d, J = 2.80 Hz, 1H), 7.32 (d, J = 8.00 Hz, 1H), 7.29 (s, 1H), 4.30 (t, |
| | J = 4.80 Hz, 2H), 4.08 (t, J = 0.00 Hz, 2H), 2.71 (s, 3H), 2.68-2.64 (m, 4H), 2.42- |
| | 2.40 (m, 2H), 2.12-2.07 (m, 1H), 2.10-1.70 (m, 4H), 1.61-1.38 (m, 5H), 1.25- |
| | 1.17 (m, 3H), 1.14-1.08 (m, 5H). |
| 440 | 1H-NMR: (400 MHz, DMSO-d6): δ 8.67 (s, 1H), 7.55 (dd, J = 2.80, 8.80 Hz, |
| | 1H), 7.46 (d, J = 2.80 Hz, 1H), 7.32 (d, J = 8.40 Hz, 1H), 7.29 (s, 1H), 4.30 (t, |
| | J = 4.80 Hz, 2H), 4.09 (t, J = 5.60 Hz, 2H), 2.67-2.77 (m, 5H), 2.51-2.64 (m, 4H), |
| | 2.40-2.42 (m, 1H), 2.05-2.12 (m, 1H), 1.70-1.74 (m, 4H), 1.54-1.64 (m, 3H), |
| | 1.38-1.49 (m, 7H), 1.23-1.10 (m, 6H). |
| 441 | 1H-NMR: (400 MHz, DMSO-d6): δ 8.67 (s, 1H), 7.55 (dd, J = 2.80, 8.80 Hz, |
| | 1H), 7.46 (d, J = 2.40 Hz, 1H), 7.31 (d, J = 3.60 Hz, 1H), 7.33 (s, 1H), 4.30 (t, |
| | J = 4.80 Hz, 2H), 4.08 (t, J = 4.40 Hz, 2H), 2. 82-2.77 (m, 1H), 2.70-2.67 (m, |
| | 4H), 2.59-2.54(m, 2H), 2.33-2.32 (m, 2H), 2.10-2.03 (m, 1H), 1.72-1.63 (m, |
| | 4H), 1.51-1.43 (m, 4H), 1.27-1.17 (m, 5H), 1.13-1.09 (m, 5H). |
| 442 | 1H-NMR: (400 MHz , DMSO-d6): δ 8.88 (s, 1H), 7.58 (dd, J = 2.40, 9.00 Hz, |
| | 1H), 7.49 (d, J = 2.40 Hz, 1H), 7.41 (s, 1H), 7.32 (d, J = 9.20 Hz, 1H), 4.33 (t, |
| | J = 4.0 Hz, 2H), 4.11 (t, J = 4.0 Hz, 2H), 3.59 (m, 2H), 3.51-3.47 (m, 1H), 3.17 |
| | (m, 1H), 2.91-2.89 (m, 1H), 2.73 (s, 3H), 2.50-2.33 (m, 1H), 2.29-2.07 (m, 6H), |
| | 1.72 (s, 3H), 1.38-1.28 (m, 5H), 1.24-1.15 (m, 4H). |
| 443 | 1H-NMR: (400 MHz, DMSO-d6): δ 8.88 (s, 1H), 7.57 (dd, J = 2.80, 8.80 Hz, |
| | 1H), 7.49 (d, J = 2.40 Hz, 1H), 7.41 (s, 1H), 7.32 (d, J = 9.20 Hz, 1H), 4.36- |
| | 4.32 (m, 2H), 4.15-4.05 (m, 2H), 3.32 (m, 2H), 3.32-2.86 (m, 4H), 2.73 (s, 3H), |
| | 2.49 (m, 1H), 2.29-2.16 (m, 3H), 2.14-2.00 (m, 2H), 1.72-1.51(m, 4H), 1.37- |
| | 1.13 (m, 9H). |
| 444 | 1H-NMR: (400 MHz, DMSO-d6): 8.77 (s, 1H), 8.36 (dd, J = 1.60, 3.00 Hz, 1H), |
| | 7.65-7.62 (m, 2H), 7.44-7.43 (m, 1H), 7.34-7.33 (m, 1H), 7.32-7.31 (m, 2H), |
| | 4.33 (s, 2H), 4.08 (s, 2H), 3.11-3.02 (m, 1H), 3.00-2.96 (m, 2H), 2.73 (s, 3H), |
| | 2.68 (s, 1H), 2.67-2.66 (m, 1H), 2.50 (s, 2H), 2.38-2.36 (m, 1H) 2.33-2.30 (m, |
| | 2H), 1.89-1.84 (m, 4H), 1.76-1.73 (m, 1H), 1.59 (s, 3H). |
| 445 | 1H-NMR: (400 MHz, DMSO-d6): 8.85 (s, 1H), 8.36 (dd, J = 1.20, 3.00 Hz, 1H), |
| | 7.62-7.57 (m, 2H), 7.45-7.43 (m, 2H), 7.35-7.29 (m, 2H), 4.33 (s, 2H), 4.08 (s, |
| | 2H), 3.17-3.13 (m, 1H), 3.10-3.01 (m, 2H), 2.70 (s, 3H), 2.66-2.65 (m, 1H), |
| | 2.50 (s, 1H), 2.35-2.29 (m, 4H), 1.89-1.86 (m, 5H), 1.67.1.66 (m, 4H), 1.23- |
| | 1.22 (m, 1H). |
| 446 | 1H-NMR: 400 MHz, DMSO-d6): 8.81 (s, 1H), 7.57 (dd, J = 2.40, 9.00 Hz, 1H), |
| | 7.51 (d, J = 2.00 Hz, 1H), 7.44-7.41 (m, 1H), 7.32 (d, J = 8.80 Hz, 1H), 6.07 (d, |
| | J = 2.00 Hz, 1H), 4.32-4.33 (m, 2H), 4.07-4.08 (m, 2H), 3.75 (s, 3H), 3.074 (s, |
| | 1H), 2.91-2.93 (m, 1H), 2.49-2.53 (m, 4H), 2.62-2.54 (m, 4H), 1.90-1.92 (m, |
| | 3H), 1.56-1.72 (m, 5H), 1.35 (m, 1H), 1.16-1.24 (m, 2H), 0.84-0.86 (m, 1H), |
| 447 | 1H-NMR: (400 MHz, DMSO-d6): δ 8.79 (s, 1H), 7.57 (dd, J = 2.80, 9.00 Hz, |
| | 1H), 7.51 (d, J = 2.00 Hz, 1H), 7.43 (t, J = 6.40 Hz, 2H), 7.31-7.34 (m, 1H), |
| | 6.05 (d, J = 2.00 Hz, 1H), 4.32 (s, 2H), 4.07 (d, J = 4.40 Hz, 2H), 3.75 (s, 3H), |
| | 3.05-2.95 (m, 2H), 2.91-2.86 (m, 1H), 2.73 (s, 4H), 2.60-2.54 (m, 1H), 2.50- |
| | 2.46 (m, 3H), 2.25-2.15 (m, 3H), 1.90 (dd, J = 9.20, Hz, 2H), 1.72-1.69 (m, |
| | 1H), 1.60-1.57 (m, 5H) |
| 448 | 1H-NMR: (400 MHz, DMSO-d6): δ 8.79 (s, 1H), 7.58 (dd, J = 2.80, 8.80 Hz, |
| | 1H), 7.43 (t, J = 4.40 Hz, 2H), 7.33 (d, J = 8.80 Hz, 1H), 6.07 (d, J = 2.40 Hz, |
| | 1H), 4.33 (s, 2H), 4.07 (d, J = 4.40 Hz, 2H), 3.75 (s, 1H), 3.08 (d, J = 8.80 Hz, |
| | 1H), 2.88 (d, J = 12.00 Hz, 1H), 2.73 (s, 1H), 2.67-2.63 (m, 1H), 0.50-2.45 (m, |
| | 4H), 2.26-2.22 (m, 3H), 1.72-1.69 (m, 1H), 1.69-1.66 (m, 3H), 1.60 (s, 3H), |
| | 1.41-1.38 (m, 2H), 1.57-1.69 (m, 5H) |
| 449 | 1H-NMR: (400 MHz, DMSO-d6): δ 8.62 (s, 1H), 7.83 (dd, J = 2.40, 8.00 Hz, |
| | 1H), 7.76 (dd, J = 4.00, 9.00 Hz, 1H), 7.58-7.51 (m, 2H), 7.50-7.31 (m, 3H), |
| | 4.33 (s, 2H), 4.09 (d, J = 4.40 Hz, 2H), 3.32-3.03 (m, 3H), 2.50-2.72 (m, 4H), |
| | 2.50-2.24 (m, 5H), 2.07 (d, J = 11.20 Hz, 2H), 1.91-1.80 (m, 5H), 1.62 (s, 3H). |
| 450 | 1H-NMR: (400 MHz, DMSO-d6): δ 8.76 (s, 1H), 7.58 (dd, J = 2.40, 9.00 Hz, |
| | 1H), 7.44 (d, J = 2.80 Hz, 1H), 7.40 (s, 1H), 7.33 (d, J = 9.20 Hz, 1H), 4.33-4.32 |
| | (m, 2H), 4.08-4.07 (m, 2H), 3.34 (s, 3H), 2.87 (d, J = 11.20 Hz, 1H), 2.79 (d, |
| | J = 10.00 Hz, 1H), 2.73 (s, 3H), 2.57-2.60 (m, 1H), 2.46 (s, 3H), 2.31-2.39 (m, |
| | 2H), 2.21-2.25 (m, 1H), 1.86-1.93 (m, 3H), 1.65-1.73 (m, 3H), 1.58 (s, 3H). |
| 451 | 1H-NMR: (400 MHz, DMSO-d6): δ 8.77 (s, 1H), 7.58 (dd, J = 2.80, 8.80 Hz, |
| | 1H), 7.44-7.41 (m, 2H), 7.33 (d, J = 9.20 Hz, 1H), 4.34-4.32 (m, 2H), 4.10-4.08 |
| | (m, 2H), 3.34 (s, 3H), 2.87 (d, J = 11.20 Hz, 1H), 2.79 (d, J = 11.60 Hz, 1H), |
| | 2.60 (s, 3H), 2.51-2.57 (m, 1H), 2.46 (s, 3H), 2.39-2.31 (m, 2H), 2.25-2.21 (m, |
| | 1H), 1.93-1.86 (m, 3H), 1.73-1.58 (m, 6H), |
| 452 | 1H-NMR: (400 MHz, DMSO-d6): δ 8.74 (s, 1H), 7.57 (dd, J = 2.40, 9.00 Hz, |
| | 1H), 7.43 (d, J = 2.80 Hz, 1H), 7.39 (s, 1H), 7.32 (d, J = 8.80 Hz, 1H), 4.32 (s, |
| | 2H), 4.07-4.08 (m, 2H), 3.92-2.94 (m, 2H), 2.71-2.73 (m, 3H), 2.53-2.56 (m, |
| | 1H), 2.44-2.51 (m, 4H), 2.17-1.70 (m, 4H), 1.23-1.70 (m, 8H) |
| 453 | 1H-NMR: (400 MHz, DMSO-d6): 8.83 (s, 1H), 7.58 (dd, J = 2.80, 8.80 Hz, 1H), |
| | 7.51 (d, J = 1.60 Hz, 2H), 7.43-7.44 (m, 1H), 7.33 (d, J = 8.80 Hz, 1H), 6.02 (d, |
| | J = 2.00 Hz, 1H), 4.33-4.32 (m, 2H), 4.08-4.07 (m, 2H), 3.74 (s, 3H), 3.00 (s, |
| | 2H), 2.73 (s, 3H), 2.67-2.68 (m, 2H), 2.50-2.54 (m, 4H), 2.25-2.33 (m, 2H), |
| | 1.91-1.86 (m, 3H), 1.62 (s, 3H), 1.58 (s, 3H). |
| 454 | 1H-NMR: (400 MHz, DMSO-d6): δ 8.82 (s, 1H), 7.58 (dd, J = 2.40, 8.80 Hz, |
| | 1H), 7.50 (d, J = 2.00 Hz, 1H), 7.43 (dd, J = 5.20, Hz, 2H), 7.33 (dd, J = 8.80, |
| | Hz, 1H), 6.02 (d, J = 1.60 Hz, 1H), 4.36-4.32 (m, 2H), 4.15-4.05 (m, 2H), 3.74 |
| | (s, 3H), 2.98 (dd, J = 9.60, 31.40 Hz, 2H), 2.73 (s, 3H), 2.59 (d, J = 11.20 Hz, |
| | 1H), 2.50 (s, 4H), 2.26-2.33 (m, 3H), 1.88 (d, J = 12.40 Hz, 3H), 1.60 (s, 3H). |
| 455 | 1H-NMR: (400 MHz, DMSO-d6): δ 8.79 (d, J = 2.40 Hz, 1H), 8.70 (d, J = 2.00 |
| | Hz, 1H), 8.21 (s, 1H), 8.08 (t, J = 2.00 Hz, 1H), 7.71 (s, 1H), 7.53 (dd, J = 2.40, |
| | 8.80 Hz, 1H), 7.38 (s, 1H), 7.29 (d, J = 9.20 Hz, 1H), 7.16 (s, 1H), 4.39- |
| | 4.34 (m, 2H), 4.15-4.09 (m, 2H), 3.06-3.04 (m, 2H), 2.67-2.71 (m, 2H), 2.50- |
| | 2.52 (m, 6H), 2.49 (s, 1H), 2.29-2.33 (m, 2H), 1.89-1.85 (m, 1H), 1.75-1.73 (m, |
| | 2H), 1.51 (s, 6H), 1.08 (s, 9H) |
| 456 | 1H-NMR: (400 MHz, DMSO-6): δ 8.79 (d, J = 2.40 Hz, 1H), 8.70 (d, J = 2.00 |
| | Hz, 1H), 8.37 (s, 1H), 8.08 (t, J = 2.00 Hz, 1H), 7.71 (s, 1H), 7.54 (dd, J = 2.80, |
| | 9.00 Hz, 1H), 7.39 (d, J = 2.00 Hz, 1H), 7.30 (d, J = 8.80 Hz, 1H), 7.22 (s, |
| | 1H), 4.36-4.32 (m, 2H), 4.15-4.05 (m, 2H), 3.06-3.04 (m, 2H), 2.63-2.68 (m, |
| | 5H), 2.54 (s, 3H), 2.38 (s, 1H), 2.33-2.35 (m, 1H), 2.27-2.32 (m, 2H), 1.92 (d, |
| | J = 10.00 Hz, 1H), 1.83 (d, J = 11.20 Hz, 2H), 1.34-1.66 (m, 6H), 1.06 (s, 9H), |
| 457 | 1H-NMR: (400 MHz, DMSO-d6): δ 8.33 (s, 1H), 7.56-7.53 (m, 1H), 7.49-7.44 |
| | (m, 1H), 7.40 (d, J = 2.80 Hz, 1H), 7.33-7.19 (m, 2H), 4.36-4.28 (m, 2H), 4.09- |
| | 4.08 (m, 2H), 3.15 (d, J = 1203.20 Hz, 1H), 3.12-3.07 (m, 1H), 2.94 (d, J = 8.00 |
| | Hz, 1H), 2.67 (s, 3H), 2.67-2.62 (m, 1H), 2.50-2.49 (m, 1H), 8 2.33 (s, 2H), 2.31 |
| | (s, 1H), 1.93-1.88 (m, 1H), 1.87 (d, J = 10.40 Hz, 1H), 1.69 (s, 3H), 1.64-1.60 |
| | (m, 2H), 1.06 (d, J = 6.40 Hz, 2H) |
| 458 | 1H-NMR: (400 MHz, DMSO-d6): δ 8.37 (s, 1H), 7.55 (dd, J = 11.60, Hz, 1H), |
| | 7.40 (d, J = 2.80 Hz, 1H), 7.30 (d, J = 8.80 Hz, 1H), 7.24 (s, 1H), 4.29-4.36 (m, |
| | 3H), 4.08 (s, 2H), 3.09-3.15 (m, 1H), 2.94 (d, J = 8.40 Hz, 1H), 2.66 (s, 3H), |
| | 2.47-2.54 (m, 2H), 2.31-2.28 (m, 1H), 2.15-2.21 (m, 1H), 8 1.93 (m, 2H), 1.74- |
| | 1.77 (m, 1H), 1.63 (s, 3H), 1.51-1.57 (m, 3H), 1.18 (d, J = 6.40 Hz, 3H), |
| 459 | 1H-NMR: (400 MHz, DMSO-d6): δ 8.94 (s, 1H), 7.59 (dd, J = 2.80, 9.00 Hz, |
| | 1H), 7.47 (s, 1H), 7.44 (d, J = 2.80 Hz, 1H), 7.34 (d, J = 8.80 Hz, 1H), 4.42-4.46 |
| | (m, 1H), 4.35 (t, J = 4.80 Hz, 2H), 4.06 (t, J = 4.80 Hz, 2H), 3.15-3.22 (m, 1H), |
| | 2.77-2.86 (m, 2H), 2.73 (s, 3H), 2.61-2.57 (m, 2H), 2.44-2.49 (m, 4H). |
| 460 | 1H-NMR: (400 MHz, DMSO-d6): δ 8.18 (s, 1H), 7.98 (s, 1H), 7.53 (dd, J = |
| | 2.40, 9.00 Hz, 1H), 7.38 (d, J = 2.00 Hz, 1H), 7.27 (d, J = 7.20 Hz, 1H), 7.13 (s, |
| | 2H), 4.28-4.27 (m, 2H), 4.09 (m, 2H), 2.79-2.67 (m, 3H), 2.51-2.49 (m, 2H), |
| | 2.34-2.32 (m, 2H), 2.24-2.11(m, 4H), 1.97 (d, J = 10.80 Hz, 2H), 1.88-1.84 (m, |
| | 1H), 1.66 (s, 6H), 1.35 (d, J = 12.00 Hz, 1H), 1.27-1.24 (m, 2H) |
| 461 | 1H-NMR: (400 MHz, DMSO-d6): δ 8.66 (s, 1H), 7.99 (s, 1H), 7.56 (dd, J = |
| | 2.80, 9.00 Hz, 1H), 7.42 (d, J = 2.40 Hz, 1H), 7.37-7.30 (m, 1H), 7.10 (s, 1H), |
| | 4.32 (s, 2H), 4.08 (s, 2H), 2.98-2.88 (m, 3H), 2.81 (m, 2H), 2.71 (s, 1H), 2.67- |
| | 2.67 (m, 1H), 2.38-2.32 (m, 4H), 2.12-2.09 (m, 1H), 1.97-1.95 (m, 1H), 1.92- |
| | 1.89 (m, 1H), 1.69-1.61 (m, 6H), 1.35 (d, J = 12.00 Hz, 1H), 1.25 (t, J = 5.20 |
| | Hz, 2H) |
| 462 | 1H-NMR: (400 MHz, DMSO-d6): δ 8.80 (s, 1H), 8.28 (m, 1H), 7.58 (dd, J = |
| | 2.80, 9.00 Hz, 1H), 7.44-7.41 (m, 2H), 7.33 (d, J = 9.20 Hz, 1H), 4.32 (t, J = |
| | 4.40 Hz, 2H), 4.07 (t, J = Hz, 2H), 3.78 (s, 3H), 2.73 (s, 3H), 2.62-2.59 (m, |
| | 2H), 2.51-2.49 (m, 3H), 2.33-2.25 (m, 2H), 1.93-1.91 (m, 3H), 1.70-1.58 (m, |
| | 6H), 1.26-1.16 (m, 3H) |
| 463 | 1H-NMR: (400 MHz, DMSO-d6): δ 8.80 (s, 1H), 8.27 (s, 1H), 7.58 (dd, J = |
| | 2.80, 9.00 Hz, 1H), 7.44-7.41 (m, 2H), 7.33 (d, J = 9.20 Hz, 1H), 4.33-4.31 (m, |
| | 2H), 4.08-4.07 (m, 2H), 3.78 (s, 3H), 2.73 (s, 3H), 2.67-2.60 (m, 2H), 2.50-2.46 |
| | (m, 2H), 2.36-2.24 (m, 3H), 1.93-1.91 (m, 3H), 1.70-1.58 (m, 6H), 1.37-1.28 |
| | (m, 1H), 1.26-1.24 (m, 2H) |
| 464 | 1H-NMR: (400 MHz, DMSO-d6): 8.65 (s, 1H), 8.37-8.38 (m, 1H), 7.63 (td, J = |
| | 1.60, 8.60 Hz, 1H), 7.56 (dd, J = 2.40, 9.00 Hz, 1H), 7.42 (d, J = 2.40 Hz, 1H), |
| | 7.29-7.34 (m, 3H), 4.29-4.30 (m, 2H), 4.07-4.08 (m, 2H), 3.19-3.22 (m, 1H), |
| | 2.90-3.01 (m, 2H), 2.67 (s, 3H), 2.50-2.51 (m, 1H), 2.43-2.50 (m, 4H), 2.22- |
| | 2.33 (m, 2H), 1.88-1.91 (m, 1H), 1.76-1.82 (m, 2H), 1.60-1.64 (m, 6H) |
| 465 | 1H-NMR: (400 MHz, DMSO-d6): 8.66 (s, 1H), 8.36-8.41 (m, 1H), 7.63 (td, J = |
| | 1.20, 8.40 Hz, 1H), 7.56 (dd, J = 2.80, 8.80 Hz, 1H), 7.42 (d, J = 2.40 Hz, 1H), |
| | 7.24-7.34 (m, 3H), 4.29 (m, 2H), 4.07-4.08 (m, 2H), 3.18-3.20 (m, 1H), 2.94- |
| | 3.01 (m, 2H), 2.67 (s, 3H), 2.51-2.64 (m, 1H), 2.44-2.50 (m, 4H), 2.32-2.34 (m, |
| | 2H), 1.87-1.91 (m, 1H), 1.76-1.79 (m, 2H), 1.61-1.67 (m, 6H) |
| 466 | 1H-NMR: (400 MHz, DMSO-d6): 8.62 (s, 1H), 8.36-8.38 (m, 1H), 7.63 (td, J = |
| | 1.60, 8.60 Hz, 1H), 7.56 (dd, J = 2.80, 8.80 Hz, 1H), 7.42 (d, J = 2.40 Hz, 1H), |
| | 7.30-7.35 (m, 3H), 4.28-4.34 (m, 2H), 4.06-4.07 (m, 2H), 3.21-3.18 (m, 1H) |
| | 2.95-3.02 (m, 2H), 2.67 (s, 3H), 2.64-2.67 (m, 1H), 2.44-2.50 (m, 4H), 2.19- |
| | 2.25 (m, 2H), 1.89-1.91 (m, 1H), 1.76-1.82 (m, 2H), 1.63-1.67 (m, 6H) |
| 467 | 1H-NMR: (400 MHz, DMSO-d6): 8.61 (s, 1H), 8.37 (dd, J = 4.80, Hz, 1H), |
| | 7.64 (td, J = 1.60, 10.20 Hz, 1H), 7.56 (dd, J = 2.40, 8.80 Hz, 1H), 7.42 (d, J = |
| | 2.40 Hz, 1H), 7.30-7.34 (m, 3H), 4.31 (m, 2H), 4.06-4.07 (m, 2H), 3.20 (m, |
| | 1H), 3.01 (dd, J = 11.20, Hz, 1H), 2.92 (dd, J = 10.00, Hz, 1H), 2.70 (s, 3H), |
| | 2.63 (s, 1H), 2.46-2.50 (m, 4H), 2.29-2.24 (m, 2H), 1.88-1.90 (m, 1H), 1.77- |
| | 1.79 (m, 2H), 1.61-1.67 (m, 6H) |
| 471 | 1H-NMR: (400 MHz, DMSO-d6): δ 8.79 (s, 1H), 7.58 (dd, J = 2.80, 8.80 Hz, |
| | 1H), 7.43 (dd, J = 5.20, Hz, 2H), 7.33 (dd, J = 9.20, Hz, 1H), 4.39-4.32 (m, |
| | 3H), 4.12-4.07 (m, 2H), 3.01-2.99 (m, 1H), 2.73 (s, 3H), 2.64-2.62 (m, 2H), |
| | 2.51-2.39 (m, 5H), 2.21-2.14 (m, 1H), 1.93-1.84 (m, 2H), 1.75-1.73 (m, 1H), |
| | 1.57 (s, 3H), 1.54-1.51 (m, 3H). |
| 485 | 1H-NMR (400 MHz, DMSO-d6): δ 8.66 (s, 1H), 7.58-7.55 (m, 1H), 7.50 (s, |
| | 1H), 7.43-7.42 (d, J = 2.40 Hz, 1H), 7.363 (s, 1H), 7.32-7.30 (d, J = 5.20 Hz, |
| | 1H), 7.28 (s, 1H), 4.32-4.30 (m, 2H), 4.08-4.07 (m, 2H), 3.75 (s, 3H), 2.94-2.89 |
| | (m, 2H), 2.71 (s, 3H), 2.62-2.61 (m, 2H), 2.50-2.49 (m, 2H), 2.29-2.16 (m, 3H), |
| | 1.90-1.86 (m, 2H), 1.67-1.55 (m, 6H), 1.30-1.29 (m, 2H) |
| 488 | 1H-NMR (400 MHz, DMSO-d6): δ 8.73 (brs, 1H), 7.83 (dd, J = 2.40, 8.20 Hz, |
| | 1H), 7.76 (dd, J = 3.60, 9.00 Hz, 1H), 7.58-7.50 (m, 2H), 7.44 (d, J = 2.40 Hz, |
| | 1H), 7.39 (s, 1H), 7.33 (d, J = 8.80 Hz, 1H), 4.35.4.32 (m, 2H), 4.10-4.08 (m, |
| | 2H), 3.32-3.03 (m, 3H), 2.72 (s, 3H), 2.65-2.50 (m, 3H), 2.50-2.25 (m, 4H), |
| | 2.08 (d, J = 11.60 Hz, 2H), 1.94-1.64 (m, 4H), 1.61 (s, 3H). |
| 491 | 1H NMR (400 MHz, DMSO-d6): δ 8.73-8.72 (m, 2H), 7.73-7.64 (m, 3H), 7.41 |
| | (s, 1H), 4.82-4.70 (m, 2H), 3.32-3.24 (m, 6H), 2.92-2.87 (m, 4H), 2.58-2.51 (m, |
| | 5H), 2.26-2.19 (m, 1H), 2.07-1.91 (m, 6H), 1.66-1.48 (m, 3H), 1.24 (s, 1H). |
| 493 | 1H-NMR (400 MHz, DMSO-d6): o 8.73 (s, 1H), 7.58 (dd, J = 2.40, 9.00 Hz, |
| | 1H), 7.45 (d, J = 2.80 Hz, 1H), 7.41 (s, 1H), 7.34-7.32 (m, 1H), 4.34-4.30 (m, |
| | 2H), 4.06 (t, J = 5.60 Hz, 2H), 3.16-3.10 (m, 1H), 2.73 (s, 3H), 2.59-2.54 (m, |
| | 1H), 2.43-2.38 (m, 1H), 2.24-2.22 (m, 5H), 2.03 (s, 3H), 1.73-1.64 (m, 8H), |
| | 1.64-1.58 (m, 9H). |
| 494 | 1H-NMR (400 MHz, DMSO-d6): o 8.53 (s, 1H), 7.56 (dd, J = 2.80, 9.00 Hz, |
| | 1H), 7.42 (d, J = 2.40 Hz, 1H), 7.31 (d, J = 9.20 Hz, 2H), 4.31-4.29 (m 2H), |
| | 4.08-4.07 (m, 2H), 2.70 (S, 3H), 2.70-2.65 (m, 2H), 2.56-2.56 (m, 2H), 2.52- |
| | 2.49 (m, 4H), 1.88 (d, J = 13.20 Hz, 3H), 1.90-1.89 (m, 1H), 1.62 (s, 3H), 1.63- |
| | 1.55 (m, 5H), 1.25-1.23 (s, 1H). |
| 495 | 1H-NMR (400 MHz, DMSO-d6 ): 8.63 (s, 1H), 7.57 (dd, J = 2.40, 9.00 Hz, |
| | 1H), 7.56 (d, J = 2.40 Hz, 1H), 7.32 (d, J = 8.80 Hz, 2H), 4.32-4.30 (m, 2H), |
| | 4.09-4.06 (m, 2H), 2.71 (s, 3H), 2.69-2.65 (m, 2H), 2.57-2.57 (m, 2H), 2.47- |
| | 2.45 (m, 4H), 2.22 (d, J = 13.60 Hz, 3H), 1.90-1.86 (m, 1H), 1.63 (s, 3H), 1.63- |
| | 1.61 (m, 5H), 1.23 (s, 1H), |
| 496 | 1H-NMR (400 MHz, DMSO-d6): δ 8.82 (s, 1H), 7.59-7.56 (m, 1H), 7.45-7.42 |
| | (m, 2H), 7.34-7.32 (d, J = 9.20 Hz, 1H), 4.35-4.32 (m, 2H), 4.07-4.06 (m, 2H), |
| | 3.17 (s, 3H), 3.12-3.10 (m, 1H), 2.85-2.74 (m, 1H), 2.69-2.67 (m, 3H), 2.65- |
| | 2.61 (m, 1H), 2.34-2.32 (m, 4H), 1.91-1.86 (m, 4H), 1.78-1.62 (m, 2H), 1.55 (s, |
| | 3H), 1.48-1.46 (m, 3H), 1.36-1.24 (m, 2H). |
| 497 | 1H-NMR (400 MHz, DMSO-d6): δ 8.81 (s, 1H), 7.59-7.57 (m, 1H), 7.45-7.43 |
| | (m, 2H), 7.33-7.31 (d, J = 9.20 Hz, 1H), 4.34-4.32 (m, 2H), 4.08-4.07 (m, 2H), |
| | 3.23-3.19 (m, 4H), 2.99-2.97 (m, 1H), 2.74 (s, 3H), 2.62-2.55 (m, 3H), 2.50- |
| | 2.49 (m, 3H), 1.91-1.89 (m, 4H), 1.62-1.57 (m, 1H), 1.56 (s, 3H), 1.40-1.34 (m, 5H). |
| 498 | 1H-NMR (400 MHz, DMSO-d6): δ 8.57 (s, 1H), 7.56 (dd, J = 2.80, 9.00 Hz, |
| | 1H), 7.42 (d, J = 2.40 Hz, 1H), 7.31 (d, J = 9.20 Hz, 2H), 4.60-4.58 (m, 1H), |
| | 4.31 (t, J = 2.00 Hz, 2H), 4.07 (t, J = 2.00 Hz, 2H), 3.07 (m, 1H), 2.96 (d, J = |
| | 9.50 Hz, 1H), 2.71 (s, 3H), 2.62-2.57 (m, 2H), 2.45-2.40 (m, 3H), 2.20-2.14 (m, |
| | 2H), 1.96-1.82 (m, 4H), 1.67-1.55 (m, 5H), 1.33-1.25 (m, 2H). |
| 499 | 1H-NMR (400 MHz, DMSO-d6): δ 8.72 (s, 1H), 7.57 (dd, J = 2.80, 9.00 Hz, |
| | 1H), 7.44 (d, J = 2.40 Hz, 1H), 7.39 (s, 1H), 7.32 (d, J = 8.80 Hz, 1H), 4.60- |
| | 4.58 (m, 1H), 4.33 (t, J = 4.80 Hz, 2H), 4.07 (t, J = 2.00 Hz, 2H), 3.00-2.95 (m, |
| | 1H), 2.93 (d, J = 9.60 Hz, 1H), 2.76-2.75(m, 1H), 2.74 (s, 3H), 2.68-2.65 (m, |
| | 1H), 2.65-2.56 (m, 1H), 2.48-2.47 (m, 1H), 2.38-2.36 (m, 1H), 2.21-2.15 (m, |
| | 1H), 2.08-2.03 (m, 1H), 1.95-1.92 (m, 2H) 1.74-1.64 (m, 2H), 1.57-1.51 (m, |
| | 5H), 1.37-1.23 (m, 2H). |
| 501 | 1H-NMR (400 MHz, DMSO-d6): δ 12.56 (s, 1H), 8.59 (s, 1H), 7.56 (dd, J = |
| | 2.40, 8.80 Hz, 1H), 7.42 (d, J = 2.40 Hz, 1H), 7.33 (d, J = 5.20 Hz, 1H), 7.30 (s, |
| | 1H), 4.60-4.32 (m, 1H), 4.31 (t, J = 2.40 Hz, 2H), 4.07 (t, J = 2.80 Hz, 2H), |
| | 3.07-3.05 (m, 1H), 2.96 (s, 1H), 2.87-2.81 (m, 1H), 2.71 (s, 3H), 2.67-2.62 (m, |
| | 2H), 2.40-2.33 (m, 2H), 2.20-2.18 (m, 1H), 2.07-2.04 (m, 1H), 1.91-1.77 (m, |
| | 4H), 1.64-1.55 (m, 5H), 1.31-1.23 (m, 2H). |
| 502 | 1H-NMR 400 MHz, DMSO-d6): 8.72 (s, 1H), 7.57 (dd, J = 2.40, 8.80 Hz, |
| | 1H), 7.44 (d, J = 2.80 Hz, 1H), 7.39 (s, 1H), 7.32 (d, J = 9.64 Hz, 1H), 4.61- |
| | 4.58 (m, 1H), 4.33 (t, J = 2.40 Hz, 2H), 4.07 (t, J = 2.00 Hz, 2H), 3.00-2.99 (m, |
| | 1H), 2.93 (d, J = 10.00 Hz, 1H), 2.76-2.75 (m, 1H), 2.73 (s, 3H), 2.68-2.64 (m, |
| | 1H), 2.56-2.55 (m, 1H), 2.50-2.49 (m, 1H), 2.38-2.36 (m, 1H), 2.21-2.15 (m, |
| | 1H), 2.08-2.02 (m, 1H), 1.94 (s, 3H), 2.74-2.64 (m, 2H), 1.57-1.51 (m, 5H), |
| | 1.37-1.31 (m, 1H). |
| 503 | 1H-NMR (400 MHz, DMSO-d6): δ 8.66 (s, 1H), 7.59-7.56 (m, 1H), 7.43 (d, |
| | J = 2.40 Hz, 1H), 7.39 (s, 1H), 7.32 (d, J = 9.20 Hz, 1H), 4.35-4.32 (m, 2H), |
| | 4.11-4.05 (m, 2H), 3.56-3.52 (m, 2H), 3.34-3.32 (m, 3H), 2.72 (s, 3H), 2.68- |
| | 2.65 (m, 3H), 2.50-249 (m, 1H), 2.34 (dd, J = 2.00, 3.60 Hz, 1H), 1.95-1.76 (m, |
| | 9H), 1.57-1.55 (m, 4H) |
| 504 | 1H-NMR (400 MHz, DMSO-d6): δ 8.65 (s, 1H), 7.58-7.56 (m, 1H), 7.43 (d, |
| | J = 2.80 Hz, 1H), 7.38 (s, 1H), 7.32 (d, J = 8.80 Hz, 1H), 4.35-4.32 (m, 2H), |
| | 4.07-4.06 (m, 2H), 3.56-3.52 (m, 2H), 3.35-3.32 (m, 3H), 2.72 (s, 3H), 2.68- |
| | 2.58 (m, 3H), 2.50-2.33 (m, 1H), 2.33-2.19 (m, 1H), 1.94-1.76 (m, 9H), 1.57 |
| | (m, 4H) |
| 505 | 1H-NMR (400 MHz, DMSO-d6): δ 8.67 (s, 1H), 7.57 (dd, J = 6.40, Hz, 1H), |
| | 7.43 (d, J = 2.40 Hz, 1H), 7.61-7.31 (m, 2H), 4.33-4.32 (m, 2H), 4.08-4.07 (m, |
| | 2H), 3.26-3.20 (m, 2H), 2.76-2.68 (m, 8H), 2.67-2.56 (m, 5H), 2.50-2.44 (m, |
| | 2H), 2.36-2.33 (m, 1H), 1.90-1.81 (m, 1H), 1.74-1.54 (m, 8H). |
| 506 | 1H-NMR (400 MHz, DMSO-d6): δ 8.70 (s, 1H), 7.57 (dd, J = 2.80, 8.80 Hz, |
| | 1H), 7.43 (d, J = 2.80 Hz, 1H), 7.38 (s, 1H), 7.32 (d, J = 8.80 Hz, 1H), 4.34- |
| | 4.31 (m, 2H), 4.08-4.05 (m, 2H), 3.26-3.20 (m, 2H), 2.76-2.69 (m, 8H), 2.62- |
| | 0.55 (m, 5H), 2.49-2.45 (m, 2H), 2.35-2.32 (m, 1H), 1.80-1.79 (m, 1H), 1.71- |
| | 1.63 (m, 8H). |
| 507 | 1H-NMR (400 MHz, DMSO-d6): δ 8.73 (s, 1H), 7.59 (dd, J = 2.40, 8.80 Hz, |
| | 1H), 7.42-7.39(m, 2H), 7.31 (d, J = 8.80 Hz, 1H), 4.33-4.32 (m, 2H), 4.11-4.10 |
| | (m, 2H), 3.88-3.83 (m, 4H), 3.39-3.15 (m, 3H), 2.77-2.75 (m, 3H), 2.72 (s, |
| | 3H), 2.55-2.50 (m, 4H), 2.38-2.37 (m, 2H), 1.66-1.61 (m, 4H), 1.64 (s, 3H), |
| | 1.34-1.24 (m, 1H). |
| 508 | 1H-NMR (400 MHz, DMSO-d6): δ 8.80 (s, 1H), 7.57 (dd, J = 2.40, 9.00 Hz, |
| | 1H), 7.44-7.40 (m, 2H), 7.32 (d, J = 8.80 Hz, 1H), 4.35-4.32 (m, 2H), 4.07-4.05 |
| | (m, 2H), 3.32-3.05 (m, 4H), 2.97-2.82 (m, 3H), 2.72 (s, 3H), 2.67-2.63 (m, 2H), |
| | 2.46-2.36 (m, 5H), 1.94-1.90 (m, 1H), 1.75-1.72 (m, 1H), 1.65 (s, 3H), 8 1.53- |
| | 1.45 (m, 4H), 1.24 (m, 1H). |
| 509 | 1H-NMR (400 MHz, DMSO-d6): δ 8.09 (s, 1H), 7.54 (dd, J = 2.80, 8.80 Hz, |
| | 1H), 7.34 (d, J = 2.80 Hz, 1H), 7.28 (d, J = 8.80 Hz, 1H), 7.05 (s, 1H), 4.31- |
| | 4.19(m, 2H), 4.18-4.09 (m, 2H), 3.22-3.19 (m, 1H), 3.15-3.03 (m, 6H), 2.64 (s, |
| | 3H), 2.56-2.55 (m, 3H), 2.51-2.50 (m, 1H), 1.86-1.75 (m, 6H), 1.74 (s, 3H), |
| | 1.36-1.16 (m, 3H). |
| 510 | 1H-NMR (400 MHz, DMSO-d6): δ 8.63 (s, 1H), 7.57 (dd, J = 2.80, 8.80 Hz, |
| | 1H), 7.37 (d, J = 2.80 Hz, 1H), 7.30 (d, J = 9.20 Hz, 1H), 7.08 (s, 1H), 4.33- |
| | 4.28 (m, 2H), 4.18-4.09 (m, 2H), 3.20-3.18 (m, 1H), 3.11-3.07 (m, 1H), 3.03- |
| | 2.88 (m, 1H), 2.84-2.81 (m, 5H), 2.65 (s, 3H), 2.49-2.46 (m, 5H), 2.39-2.30 (m, |
| | 1H), 1.89-1.80 (m, 4H), 1.73 (s, 3H), 1.42-1.39 (m, 1H), 1.24 (m, 1H). |
| 511 | 1H-NMR (400 MHz, DMSO-d6): δ 8.55 (s, 1H), 7.56 (dd, J = 2.80, 9.00 Hz, |
| | 1H), 7.43-7.42 (m, 1H), 7.31 (d, J = 8.80 Hz, 2H), 4.32 (t, J = 4.80 Hz, 2H), |
| | 4.07 (t, J = 4.80 Hz, 2H), 3.01-2.73 (m, 4H), 2.71 (s, 3H), 2.67-2.55 (m, 3H), |
| | 2.46-2.39 (m, 3H), 2.31-2.13 (m, 3H), 2.02-1.86 (m, 3H), 1.62-1.52 (m, 6H), |
| | 1.38-1.31 (m, 2H). |
| 512 | 1H-NMR (400 MHz, DMSO-d6): δ 8.70 (d, J = 5.20 Hz, 1H), 7.57 (dd, J = |
| | 2.40, 8.80 Hz, 1H), 7.44 (t, J = 2.40 Hz, 1H), 7.39 (s, 1H), 7.32 (d, J = 8.80 Hz, |
| | 1H), 4.33 (t, J = 4.80 Hz, 2H), 4.07 (t, J = 4.80 Hz, 2H), 2.99-2.73 (m, 4H), |
| | 2.73 (s, 3H), 2.67-2.53 (m, 3H), 2.48-2.37 (m, 3H), 2.32-2.11 (m, 3H), 2.05- |
| | 1.81 (m, 3H), 1.71-1.51 (m, 6H), 1.38-1.32 (m, 2H). |
| 513 | 1H-NMR (400 MHz, DMSO-d6): o 8.44 (s, 1H), 7.55 (dd, J = 2.80, 9.00 Hz, |
| | 1H), 7.41 (d, J = 2.80 Hz, 1H), 7.31-7.27 (m, 2H), 4.30 (t, J = 4.80 Hz, 2H), |
| | 4.09-4.07 (m, 2H), 3.28-3.08 (m, 5H), 2.87-2.81 (m, 1H), 2.77-2.73 (m, 1H), |
| | 2.72-2.68 (m, 4H), 2.65-2.59 (m, 1H), 2.46-2.42 (m, 3H), 2.31-2.11 (m, 3H), |
| | 1.89-1.78 (m, 1H), 1.71-1.51 (m, 6H), 1.48-1.31 (m, 2H). |
| 515 | 1H-NMR (400 MHz, DMSO-d6): δ 12.99 (s, 1H), 8.31 (s, 1H), 7.53 (dd, J = |
| | 2.80, 8.80 Hz, 1H), 7.39 (d, J = 2.80 Hz, 1H), 7.28 (d, J = 8.80 Hz, 1H), 7.18 (s, |
| | 1H), 4.33 (t, J = 4.00 Hz, 2H), 4.29 (t, J = 4.40 Hz, 2H), 2.80-2.79 (m, 1H), |
| | 2.67 (s, 3H), 2.68-2.51 (m, 2H), 2.40-2.37 (m, 1H), 2.32-2.28 (m, 1H), 2.15- |
| | 2.11 (m, 1H), 1.88-1.82 (m, 3H), 1.64-1.48 (m, 6H), 1.25-1.23 (m, 3H), 0.92 (s, 3H). |
| 516 | 1H-NMR (400 MHz, DMSO-d6): δ 13.48 (s, 1H), 8.56 (s, 1H), 7.55 (dd, J = |
| | 2.80, 8.80 Hz, 1H), 7.42 (d, J = 2.80 Hz, 1H), 7.30 (d, J = 4.80 Hz, 2H), 4.36 (t, |
| | J = 4.40 Hz, 2H), 4.33 (t, J = 5.20 Hz, 2H), 2.81-2.79 (m, 1H), 2.71-2.66 (m, |
| | 4H), 2.62-2.51 (m, 2H), 2.49-2.36 (m, 2H), 2.34-2.26 (m, 1H), 2.14-2.10 (m, |
| | 1H), 1.90-1.84 (m, 3H), 1.66-1.48 (m, 3H), 1.34-1.23 (m, 4H), 0.92 (s, 3H). |
| 517 | 1H-NMR (400 MHz, DMSO-d6): δ 8.48 (s, 1H), 7.55 (dd, J = 2.80, 8.80 Hz, |
| | 1H), 7.41 (d, J = 2.80 Hz, 1H), 7.32-7.28 (m, 2H), 4.32-4.29 (m, 2H), 4.97-4.07 |
| | (m, 2H), 3.62-3.60 (m, 2H), 3.17-3.08 (m, 2H), 2.68 (s, 3H), 2.67-2.54 (m, 6H), |
| | 2.51-2.49 (m, 6H), 2.23-2.17 (m, 1H), 1.89-1.78 (m, 3H), 1.63-1.50 (m, 6H). |
| 518 | 1H-NMR (400 MHz, DMSO-d6): δ 8.49 (s, 1H), 7.55 (dd, J = 2.40, 8.80 Hz, |
| | 1H), 7.41 (d, J = 2.40 Hz, 1H), 7.32-7.28 (m, 2H), 4.32-4.29 (m, 2H), 4.09-4.07 |
| | (m, 2H), 3.62-3.51 (m, 2H), 3.17-3.08 (m, 2H), 2.71 (s, 3H), 2.68-2.67 (m, 6H), |
| | 2.54-2.49 (m, 6H), 2.23-2.11 (m, 1H), 1.89-1.73 (m, 3H), 1.58-1.50 (m, 6H). |
| 519 | 1H-NMR (400 MHz, DMSO-d6): δ 8.55 (brs, 1H), 7.54 (dd, J = 2.40, 8.80 |
| | Hz, 1H), 7.41 (d, J = 2.80 Hz, 1H), 7.31 (d, J = 3.20 Hz, 1H), 7.29 (brs, 1H), |
| | 4.30 (dd, J = 4.80, 8.4 Hz, 2H), 4.07 (s, 2H), 3.18 (s, 3H), 2.76 (s, 1H), 2.69 (s, |
| | 3H), 2.63-2.60 (m, 3H), 2.46-2.40 (m, 4H), 2.25-2.19 (m, 1H), 2.05 (d, J = 10.8 |
| | Hz, 1H), 1.84-1.78 (m, 2H), 1.66-1.55 (m, 5H), 1.23 (d, J = 3.6 Hz, 1H), 1.12 |
| | (d, J = 6.4 Hz, 1H), 0.56-0.55 (m, 1H), 0.35-0.34 (m, 1H). |
| 520 | 1H-NMR (400 MHz, DMSO-d6): δ 8.58 (brs, 1H), 7.55 (dd, J = 2.40, 8.80 Hz, |
| | 1H), 7.42 (d, J = 2.80 Hz, 1H), 7.32 (d, J = 2.00 Hz, 1H), 7.29 (brs, 1H), 4.33 |
| | (d, J = 10.4 Hz, 2H), 4.06 (s, 2H), 3.18 (s, 3H), 2.76 (s, 1H), 2.70 (s, 3H), 2.63- |
| | 2.60 (m, 3H), 2.46-2.40 (m, 4H), 2.25-2.19 (m, 1H), 2.05 (d, J = 10.8 Hz, 1H), |
| | 1.84-1.78 (m, 2H), 1.66-1.55 (m, 5H), 1.23 (d, J = 3.6 Hz, 1H), 1.12 (d, J = 6.4 |
| | Hz, 1H), 0.55 (d, J = 3.6 Hz, 1H), 0.31-0.29 (m, 1H). |
| 521 | 1H-NMR (400 MHz, DMSO-d6): δ 8.59 (brs, 1H), 7.55 (dd, J = 2.40, 8.80 Hz, |
| | 1H), 7.42 (d, J = 2.40 Hz, 1H), 7.32 (d, J = 2.40 Hz, 1H), 7.29 (brs, 1H), 4.31 |
| | (s, 2H), 4.06 (s, 2H), 3.19 (s, 3H), 2.76 (s, 1H), 2.70 (s, 3H), 2.67-2.60 (m, 3H), |
| | 2.46-2.40 (m, 4H), 2.25-2.19 (m, 1H), 2.05 (d, J = 10.8 Hz, 1H), 1.84-1.78 (m, |
| | 2H), 1.66-1.55 (m, 5H), 1.23 (d, J = 3.6 Hz, 1H), 1.12 (d, J = 6.4 Hz, 1H), 0.55 |
| | (t, J = 3.6 Hz, 1H), 0.35-0.34 (m, 1H). |
| 522 | 1H-NMR (400 MHz, DMSO-d6): δ 8.61 (brs, 1H), 7.55 (dd, J = 2.80, 8.80 Hz, |
| | 1H), 7.42 (d, J = 2.40 Hz, 1H), 7.32 (d, J = 4.80 Hz, 1H), 7.29 (brs, 1H), 4.30 (t, |
| | J = 4.80 Hz, 2H), 4.06 (s, 2H), 3.19 (s, 3H), 2.76 (s, 1H), 2.70 (s, 3H), 2.67-2.60 |
| | (m, 3H), 2.46-2.40 (m, 4H), 2.20-2.10 (m, 1H), 1.99 (d, J = 10.8 Hz, 1H), 1.84- |
| | 1.78 (m, 2H), 1.66-1.55 (m, 5H), 1.23 (d, J = 3.6 Hz, 1H), 1.13 (d, J = 6.4 Hz, |
| | 1H), 0.54 (d, J = 5.2 Hz, 1H), 0.35-0.34 (m, 1H). |
| 523 | 1H-NMR (400 MHz, DMSO-d6): δ 8.74 (s, 1H), 7.57 (dd, J = 2.40, 9.00 Hz, |
| | 1H), 7.44 (d, J = 2.40 Hz, 1H), 7.39 (s, 1H), 7.32 (d, J = 8.80 Hz, 1H), 4.32- |
| | 4.31 (m, 2H), 4.08-4.05 (m, 2H), 3.82-3.80 (m, 1H), 3.72-3.71 (m, 1H), 3.52- |
| | 3.46 (m, 1H), 2.92-2.90 (m, 1H), 2.72-2.70 (m, 4H), 2.51-2.50 (m, 2H), 2.50- |
| | 2.49 (m, 4H), 2.32-2.31 (m, 1H), 2.22-2.21 (m, 1H), 2.05-2.03 (m, 1H), 1.94- |
| | 1.92(m, 1H), 1.59-1.56 (m, 4H). |
| 524 | 1H-NMR (400 MHz, DMSO-d6): δ 8.82 (s, 1H), 7.58 (dd, J = 2.80, 8.80 Hz, |
| | 1H), 7.45-7.44 (m, 2H), 7.33 (d, J = 9.20 Hz, 1H), 4.34-4.33 (m, 2H), 4.07-4.05 |
| | (m, 2H), 3.70-3.58 (m, 2H), 3.51 (m, 1H), 2.86-2.84 (m, 2H), 2.74 (s, 3H), |
| | 2.50-2.51 (m, 3H), 2.50-2.49 (m, 4H), 2.27-2.18 (m, 3H), 1.58-1.54 (m, 4H), |
| 525 | 1H-NMR (400 MHz, DMSO-d6): δ 8.37 (s, 1H), 7.54 (dd, J = 2.40, 9.00 Hz, |
| | 1H), 7.40 (d, J = 2.40 Hz, 1H), 7.30 (d, J = 8.80 Hz, 1H), 7.23 (s, 1H), 4.31- |
| | 4.29 (m, 2H), 4.09-4.08 (m, 2H), 3.81 (d, J = 10.80 Hz, 1H), , 3.79-3.70 (m, |
| | 1H), 3.51-3.48 (m, 1H), 2.91 (d, J = 11.20 Hz, 1H), 2.68-2.67 (m, 4H), 2.52- |
| | 2.50 (m, 2H), 2.50-2.49 (m, 4H), 2.31-2.30 (m, 1H), 2.28-2.27(m, 1H), 2.20- |
| | 2.19 (m, 1H), 1.92 (dd, J = 10.80, Hz, 1H), 1.64 (s, 3H), 1.53-1.52 (m, 1H). |
| 526 | 1H-NMR (400 MHz, DMSO-d6): δ 8.87 (s, 1H), 7.58 (dd, J = 2.40, 8.80 Hz, |
| | 1H), 7.43 (d, J = 2.40 Hz, 1H), 7.31-7.27 (m, 2H), 4.32-4.30 (m, 2H), 4.07-4.05 |
| | (m, 2H), 3.82 (d, J = 10.80 Hz, 1H), 3.68 (t, J = 8.00 Hz, 1H), 3.51 (t, J = 9.60 |
| | Hz, 1H), 2.82 (t, J = 11.60 Hz, 2H), 2.69 (s, 3H), 2.68-2.67 (m, 2H), 2.51-2.50 |
| | (m, 4H), 2.33-2.32 (m, 2H), 2.26-2.20 (m, 1H), 1.94-1.91 (m, 1H), 1.63 (s, 3H), |
| | 1.60-1.53 (m, 1H). |
| 527 | 1H-NMR (400 MHz, DMSO-d6): δ 8.26 (s, 1H), 7.53 (dd, J = 2.80, 8.80 Hz, |
| | 1H), 7.39 (d, J = 2.80 Hz, 1H), 7.29 (d, J = 8.80 Hz, 1H), 7.20-7.17 (m, 1H), |
| | 4.30-4.28 (m, 2H), 4.09-4.08 (m, 2H), 3.51-3.50 (m, 2H), 2.69 (s, 3H), 2.66- |
| | 2.64 (m, 2H), 2.63-2.60 (m, 4H), 2.52-2.48 (m, 6H), 2.33-2.19 (m, 1H), 1.88- |
| | 1.79 (m, 3H), 1.66-1.62 (m, 4H), 8 1.46-1.41 (m, 2H), 1.28 (s, 1H) |
| 528 | 1H-NMR (400 MHz, DMSO-d6): o 8.39 (s, 1H), 7.54 (dd, J = 2.80, 9.00 Hz, |
| | 1H), 7.40 (d, J = 2.80 Hz, 1H), 7.31-7.23 (m, 2H), 4.31-4.29 (m, 2H), 4.09-4.08 |
| | (m, 2H), 3.57-3.48 (m, 2H), 2.65 (s, 3H), 2.61-2.60 (m, 2H), 2.54-2.51 (m, 4H), |
| | 2.50-2.49 (m, 6H), 2.33-2.32 (m, 1H), 1.88-1.80 (m, 3H), 1.64-1.60 (m, 4H), |
| | 1.44-1.43 (m, 2H), 1.25 (s, 1H). |
| 529 | 1H-NMR (400 MHz, DMSO-d6): o 7.92 (s, 1H), 7.51 (dd, J = 8.80, 2.40 Hz, |
| | 1H), 7.36 (d, J = 2.40 Hz, 1H), 7.26 (d, J = 9.20 Hz, 1H), 7.02 (s, 1H), 4.26 (t, |
| | J = 4.80 Hz, 2H), 4.07 (t, J = 16.00 Hz, 2H), 3.42-3.41 (m, 2H), 3.21-3.16 (m, |
| | 2H), 2.92 (s, 2H), 2.83-2.75 (m, 2H), 2.59-2.49 (m, 6H), 2.20-2.28 (m, 3H), |
| | 2.09-2.08 (m, 1H), 1.90-1.85 (m, 1H), 1.70 (s, 3H), 1.60-1.53 (m, 5H) ppm. |
| 530 | 1H-NMR (400 MHz, DMSO-d6): δ 8.38 (s, 1H), 7.54 (dd, J = 2.40, 8.80 Hz, |
| | 1H), 7.39 (d, J = 2.40 Hz, 1H), 7.28 (d, J = 8.80 Hz, 1H), 7.21 (s, 1H), 4.30 (m, |
| | 2H), 4.08 (m, 2H), 3.10-3.07 (m, 1H), 2.98-2.95 (m, 1H), 2.70 (s, 4H), 2.54- |
| | 2.52 (m, 1H), 2.31-2.12 (m, 5H), 1.84-1.60 (m, 6H), 1.43-1.41 (m, 1H), 1.24- |
| | 1.20 (m, 2H), 0.93 (s, 3H). |
| 531 | 1H-NMR (400 MHz, DMSO-d6): δ 8.57 (s, 1H), 7.55 (dd, J = 2.80, 9.00 Hz, |
| | 1H), 7.42 (d, J = 2.80 Hz, 1H), 7.31-7.29 (m, 2H), 4.32 (m, 2H), 4.08-4.07 (m, |
| | 2H), 3.10-3.07 (m, 1H), 2.98-2.95 (m, 1H), 2.71 (s, 3H), 2.67-2.61 (m, 2H), |
| | 2.49-2.10 (m, 5H), 1.91-1.60 (m, 4H), 1.58 (s, 3H), 1.49-1.46 (m, 1H), 1.27- |
| | 1.23 (m, 1H), 0.94 (s, 3H). |
| 532 | 1H-NMR (400 MHz, DMSO-d6): o 8.67 (s, 1H), 7.58 (dd, J = 2.80, 8.80 Hz, |
| | 1H), 7.42-7.40 (m, 2H), 7.36-7.32 (m, 3H), 4.35 (t, J = 4.40 Hz, 2H), 4.08-4.06 |
| | (m, 2H), 4.02-3.85 (m, 4H), 2.85-2.81 (m, 1H), 2.72 (s, 3H), 2.62-2.54 (m, 2H), |
| | 2.47-2.35 (m, 2H), 2.06-1.92 (m, 1H), 1.82-1.69 (m, 1H), 1.56 (s, 3H). |
| 533 | 1H-NMR (400 MHz, DMSO-d6): δ 8.70 (s, 1H), 7.58 (dd, J = 2.80, 9.00 Hz, |
| | 1H), 7.43-7.41 (m, 2H), 7.36-7.32 (m, 3H), 4.35 (t, J = 4.40 Hz, 2H), 4.24-4.21 |
| | (m, 2H), 4.12-3.99 (m, 2H), 3.88-3.85 (m, 2H), 2.87-2.79 (m, 1H), 2.73 (s, 3H), |
| | 2.61-2.53 (m, 2H), 2.45-2.35 (m, 2H), 2.03-1.87 (m, 1H), 1.84-1.71 (m, 1H), |
| | 1.58 (s, 3H). |
| 534 | 1H-NMR (400 MHz, DMSO-d6): δ 8.80-8.76 (m, 2H), 7.73 (d, J = 8.40 Hz, |
| | 1H), 7.68-7.67 (m, 2H), 7.46 (d, J = 4.80 Hz, 1H), 4.74 (s, 2H), 4.46-4.41 |
| | (m, 1H), 2.85-2.77 (m, 2H), 2.68-2.66 (m, 1H), 2.55 (m, 3H), 2.49-2.42 (m, |
| | 2H), 2.20-2.19 (m, 1H), 2.00-1.92 (m, 6H), 1.89-1.65 (s, 3H). |
| 535 | 1H-NMR (400 MHz, DMSO-d6): δ 8.77 (d, J = 4.40 Hz, 2H), 7.73 (d, J = |
| | 8.40 Hz, 1H), 7.68-7.65 (m, 2H), 7.46 (d, J = 4.80 Hz, 1H), 4.73 (s, 2H), 4.45- |
| | 4.42 (m, 1H), 2.85-2.77 (m, 2H), 2.68-2.66 (m, 1H), 2.55-2.52 (m, 3H), 2.50- |
| | 2.46 (m, 2H), 2.19-2.18 (m, 1H), 1.97-1.92 (m, 3H), 1.89 (s, 3H), 1.73-1.66 (m, 3H). |
| 536 | 1H-NMR (400 MHz, DMSO-d6): δ 13.48 (s, 1H), 8.56 (s, 1H), 7.55 (dd, J = |
| | 2.80, 8.80 Hz, 1H), 7.42 (d, J = 2.80 Hz, 1H), 7.30 (d, J = 4.80 Hz, 2H), 4.36 (t, |
| | J = 4.40 Hz, 2H), 4.33 (t, J = 5.20 Hz, 2H), 2.81-2.79 (m, 1H), 2.71-2.66 (m, |
| | 4H), 2.62-2.51 (m, 2H), 2.49-2.36 (m, 2H), 2.34-2.26 (m, 1H), 2.14-2.10 (m, |
| | 1H), 1.90-1.84 (m, 3H), 1.66-1.48 (m, 3H), 1.34-1.23 (m, 4H), 0.92 (s, 3H). |
| 537 | 1H-NMR (400 MHz, DMSO-d6): δ 8.83-8.80 (m, 2H), 7.72 (d, J = Hz, 1H), |
| | 7.68-7.65 (m, 2H), 7.52 (d, J = 4.80 Hz, 1H), 4.73 (s, 2H), 2.73 (m, 1H), 2.64- |
| | 2.55 (m, 4H), 2.50-2.45 (m, 2H), 2.20-2.10 (m, 1H), 1.91 (s, 1H), 1.89 (s, 3H), |
| | 1.62-1.53 (m, 5H), 1.28-1.24 (m, 3H). |
| 538 | 1H-NMR (400 MHz, DMSO-d6): δ 8.83 (s, 1H), 8.80 (d, J = 4.80 Hz, 1H), |
| | 7.73 (d, J = 8.80 Hz, 1H), 7.68-7.65 (m, 2H), 7.52 (d, J = 4.80 Hz, 1H), 4.73 (s, |
| | 2H), 2.67-2.61 (m, 3H), 2.52-2.51 (m, 2H), 2.43-2.42 (m, 2H), 2.21-2.17 (m, |
| | 1H), 1.93-1.91 (m, 1H), 1.90 (s, 3H), 1.62-1.52 (m, 5H), 1.27-1.24 (m, 3H). |
| 539 | 1H-NMR (400 MHz, DMSO-d6): δ 8.18 (s, 1H), 7.52 (dd, J = 2.40, 8.80 Hz, |
| | 1H), 7.36 (s, 1H), 7.25 (d, J = 8.80 Hz, 1H), 7.07 (s, 1H), 4.25-4.26 (m, 2H), |
| | 4.09-4.08 (m, 2H), 3.50 (s, 1H), 2.67 (s, 3H), 2.61-2.51 (m, 2H), 2.33-2.28 (m, |
| | 2H), 2.12-2.08 (m, 1H), 1.81-1.79 (m, 1H), 1.65-1.61 (m, 4H), 1.46-1.24 (m, |
| | 6H), 1.00-0.98 (m, 1H), 0.92-0.84 (m, 4H), |
| 540 | 1H-NMR (400 MHz, DMSO-d6): δ 7.81 (s, 1H), 7.48 (dd, J = 2.8 Hz, 8.8 Hz, |
| | 1H), 7.34-7.33 (m, 2H), 7.24 (d, J = 8.80 Hz, 1H), 7.17 (d, J = 7.60 Hz, 1H), |
| | 7.14-7.11 (m, 1H), 7.01-6.99 (m, 2H), 4.23-4.22 (m, 2H), 4.04-4.03 (m, 2H), |
| | 3.55 (d, J = 9.60 Hz, 1H), 3.04-3.02 (m, 1H), 2.51-2.49 (m, 3H), 2.33-2.32 (m, |
| | 1H), 2.25-2.19 (m, 2H), 2.18-2.14 (m, 1H), 2.08-2.01 (m, 1H), 2.05-1.95 (m, |
| | 1H), 1.92-1.71 (m, 1H), 1.63-1.48 (m, 6H), 1.47-1.38 (m, 3H), 1.34 (s, 1H). |
| 541 | 1H-NMR (400 MHz, DMSO-d6): δ 7.81 (s, 1H), 7.48 (dd, J = 2.8 Hz, 8.8 Hz, |
| | 1H), 7.34-7.28 (m, 2H), 7.23 (d, J = 8.80 Hz, 1H), 7.17 (d, J = 7.60 Hz, 1H), |
| | 7.14-7.11 (m, 1H), 7.01-6.96 (m, 2H), 4.23-4.22 (m, 2H), 4.04-4.03 (m, 2H), |
| | 3.58 (d, J = 9.60 Hz, 1H), 3.04-3.02 (m, 1H), 2.51-2.49 (m, 3H), 2.33-2.32 (m, |
| | 2H), 2.25-2.19 (m, 1H), 2.18-2.14 (m, 1H), 2.08-2.01 (m, 1H), 2.05-1.95 (m, |
| | 1H), 1.92-1.71 (m, 1H), 1.63-1.48 (m, 6H), 1.47-1.38 (m, 3H), 1.34 (s, 1H). |
| 542 | 1H-NMR (400 MHz, DMSO-d6): δ 8.01 (s, 1H), 7.52 (dd, J = 2.8 Hz, 8.8 Hz, |
| | 1H), 7.37 (d, J = 2.4 Hz, 1H), 7.33-7.26 (m, 2H), 7.17-7.11 (m, 3H), 6.99-6.94 |
| | (m, 1H), 4.29-4.24 (m, 2H), 4.05-3.99 (m, 2H), 3.50 (d, J = 7.40 Hz, 1H), 2.60 |
| | (d, J = 19.20 Hz, 1H), 2.50-2.42 (m, 3H), 2.49-2.41 (m, 2H), 2.37-2.33 (m, 2H), |
| | 2.32-2.26 (m, 2H), 1.77-1.59 (m, 7H), 1.63-1.45 (m, 3H), 1.40 (s, 1H) |
| 543 | 1H-NMR (400 MHz, DMSO-d6): δ 8.15 (s, 1H), 7.54 (dd, J = 2.8 Hz, 8.8 Hz, |
| | 1H), 7.40 (d, J = 2.0 Hz, 1H), 7.34-7.28 (m, 3H), 7.18-7.12 (m, 2H), 6.99-6.95 |
| | (m, 1H), 4.31-4.26 (m, 2H), 4.04-3.99 (m, 2H), 3.50-3.40 (d, J = 7.40 Hz, 1H), |
| | 2.84-2.80 (d, J = 12.60 Hz, 1H), 2.54 (s, 3H), 2.38-2.32 (m, 2H), 2.26-2.21 (m, |
| | 2H), 2.18-1.99 (m, 2H), 1.72-1.52 (m, 9H), 1.40 (s, 2H) |
| 544 | 1H-NMR (400 MHz, DMSO-d6): δ 8.72 (s, 1H), 7.58-7.55 (dd, J = 2.40, 8.80 |
| | Hz, 1H), 7.44 (d, J = 2.80 Hz, 1H), 7.36 (s, 1H), 7.32 (d, J = 8.80 Hz, 1H), |
| | 4.36-4.27 (m, 2H), 4.08-4.04 (m, 2H), 2.85-2.83 (m, 1H), 2.79-2.69 (m, 5H), |
| | 2.67-2.55 (m, 3H), 2.26-2.20 (m, 1H), 2.15-1.97 (m, 4H), 1.81-1.49 (m, 4H), |
| | 1.38-1.31 (m, 1H), 1.30-1.22 (s, 2H), 1.15 (d, J = 6.40 Hz, 1H), 0.81-0.62 (m, 3H). |
| 545 | 1H-NMR (400 MHz, DMSO-d6): δ 8.68 (s, 1H), 7.58-7.55 (dd, J = 2.40, 8.80 |
| | Hz, 1H), 7.44 (d, J = 2.80 Hz, 1H), 7.34 (s, 1H), 7.32 (d, J = 8.80 Hz, 1H), |
| | 4.35-4.29 (m, 2H), 4.08-4.04 (m, 2H), 2.85-2.83 (m, 1H), 2.79-2.69 (m, 5H), |
| | 2.67-2.55 (m, 3H), 2.26-2.20 (m, 1H), 2.15-1.97 (m, 3H), 1.81-1.49 (m, 4H), |
| | 1.38-1.31 (m, 1H), 1.30-1.22 (s, 2H), 1.15 (d, J = 6.40 Hz, 1H), 0.79-0.71 (m, |
| | 2H), 0.68-0.67 (m, 2H). |
| 546 | 1H-NMR (400 MHz, DMSO-d6): δ 8.48 (s, 1H), 7.63 (d, J = 3.2 Hz, 1H), 7.60 |
| | (d, J = 3.2 Hz, 1H), 7.55 (dd, J = 2.4 Hz, 8.8 Hz, 1H), 7.42 (d, J = 2.8 Hz, 1H), |
| | 7.30-7.28 (m, 2H), 4.31-4.25 (m, 2H), 4.12-4.04 (m, 3H), 2.68-2.51 (m, 1H), |
| | 2.50-2.49 (m, 3H), 2.43-2.40 (m, 3H), 2.35-2.34 (m, 1H), 2.33-2.32 (m, 1H), |
| | 2.33-2.31 (m, 1H), 2.33-2.28 (m, 1H), 2.26-. 89 (m, 1H), 1.78-1.61 (m, 3H), |
| | 1.55 (s, 3H), 1.48-1.35 (m, 3H), |
| 547 | 1H-NMR (400 MHz, DMSO-d6): δ 8.44 (s, 1H), 7.64 (d, J = 3.20 Hz, 1H), 7.61 |
| | (d, J = 3.20 Hz, 1H), 7.56 (dd, J = 3.2 Hz, 8.8 Hz, 1H), 7.42 (d, J = 2.80 Hz, |
| | 1H), 7.35 (s, 1H), 7.32 (d, J = 9.2 Hz, 1H), 4.35-4.28 (m, 2H), 4.11-4.03 (m, |
| | 3H), 2.67-2.60 (m, 1H), 2.56-2.49 (m, 3H), 2.49-2.42 (m, 2H), 2.39-2.33 (m, |
| | 2H), 2.29-2.26 (m, 2H), 2.24-2.17 (m, 6H), 1.85-1.76 (m, 5H) |
| 548 | 1H-NMR (400 MHz, DMSO-d6): δ 8.48 (s, 1H), 7.64 (d, J = 3.20 Hz, 1H), 7.61 |
| | (d, J = 3.20 Hz, 1H), 7.55 (dd, J = 2.8 Hz, 9.20 Hz, 1H), 7.43 (d, J = 2.80 Hz, |
| | 1H), 7.38 (s, 1H), 7.33 (d, J = 9.20 Hz, 1H), 4.35-4.31 (m, 2H), 4.30-4.26 (m, |
| | 3H), 2.70-2.67 (m, 1H), 2.57-2.50 (m, 3H), 2.49-2.42 (m, 2H), 2.38-2.33 (m, |
| | 2H), 2.25-2.21 (m, 2H), 1.81-1.75 (m, 6H), 1.67-1.50 (m, 5H). |
| 549 | 1H-NMR (400 MHz, DMSO-d6): δ 8.38 (s, 1H), 7.62 (d, J = 3.20 Hz, 1H), 7.59 |
| | (d, J = 3.20 Hz, 1H), 7.54 (dd, J = 2.8 Hz, 8.0 Hz, 1H), 7.40 (d, J = 2.40 Hz, |
| | 1H), 7.30-7.28 (m, 2H), 4.31-4.24 (m, 2H), 4.12-4.02 (m, 3H), 2.67-2.52 (m, |
| | 1H), 2.52-2.50 (m, 3H), 2.43-2.34 (m, 3H), 2.33-2.32 (m, 2H), 2.32-2.28 (m, |
| | 1H), 2.28-1.90 (m, 1H), 1.69-1.52 (m, 8H), 1.49 (s, 2H). |
| 550 | 1H-NMR (400 MHz, DMSO-d6): δ 8.76 (d, J = 42.40 Hz, 2H), 7.73 (d, J = |
| | 8.40 Hz, 1H), 7.64-7.67 (m, 2H), 7.48 (d, J = 4.80 Hz, 1H), 4.74 (s, 2H), 2.60- |
| | 2.63 (m, 4H), 2.45-2.42 (m, 3H), 2.19-2.15 (m, 1H), 1.91 (s, 4H), 1.63-1.49 (m, |
| | 5H), 1.26-1.21 (m, 3H). |
| 551 | 1H-NMR (400 MHz, DMSO-d6): δ 8.52 (s, 1H), 7.56 (dd, J = 2.40, 9.00 Hz, |
| | 1H), 7.42 (d, J = 2.80 Hz, 1H), 7.32-7.30 (m, 2H), 4.31 (t, J = 4.80 Hz, 2H), |
| | 4.08 (t, J = 4.40 Hz, 2H), 3.71-3.64 (m, 1H), 3.47 (s, 3H), 3.00-2.90 (m, 2H), |
| | 2.70 (s, 3H), 2.52- 2.44 (m, 4H), 2.19-2.07 (m, 3H), 1.89-1.85 (m, 1H), 1.70- |
| | 1.62 (m, 4H), 1.58 (s, 3H), 1.48-1.40 (m, 2H). |
| 552 | 1H-NMR (400 MHz, DMSO-d6): δ 8.56 (s, 1H), 7.56 (dd, J = 2.80, 9.00 Hz, |
| | 1H), 7.42 (d, J = 2.40 Hz, 1H), 7.32-7.30 (m, 2H), 4.31 (t, J = 4.80 Hz, 2H), |
| | 4.08 (t, J = 4.40 Hz, 2H), 3.70-3.64 (m, 1H), 3.47 (s, 3H), 2.99-2.91 (m, 2H), |
| | 2.89 (s, 3H), 2.52-2.49 (m, 4H), 2.23-2.08 (m, 3H), 1.90-1.85 (m, 2H), 1.70- |
| | 1.61 (m, 3H), 1.58 (s, 3H), 1.43-1.37 (m, 2H). |
| 553 | 1H-NMR (400 MHz, DMSO-d6): δ 8.15 (brs, 1H), 7.52 (dd, J = 2.80, 8.80 Hz, |
| | 1H), 7.38 (d, J = 2.40 Hz, 1H), 7.26 (d, J = 9.20 Hz, 1H), 7.09 (s, 1H), 4.26 (t, |
| | J = 4.00 Hz, 2H), 4.13 (t, J = 6.00 Hz, 1H), 4.03 (t, J = 8.40 Hz, 1H), 2.78-2.77 |
| | (m, 3H), 2.67-2.60 (m, 4H), 2.49-2.46 (m, 3H), 2.40-2.36 (m, 1H), 2.27-2.22 |
| | (m, 1H), 2.12-2.08 (m, 1H), 1.84-1.81 (m, 1H), 1.65-1.58 (m, 3H), 1.42-1.39 |
| | (m, 3H), 1.14-1.03 (m, 2H), 0.92 (s, 3H), |
| 554 | 1H-NMR (400 MHz, DMSO-d6): δ 12.9 (brs, 1H), 8.54 (brs, 1H), 7.91 (s, |
| | 1H), 7.49 (dd, J = 4.00, 8.00 Hz, 1H), 7.35-7.34 (m, 1H), 7.27-7.22 (m, 1H), |
| | 6.97 (s, 1H), 4.27-4.23 (m, 2H), 4.15-4.06 (m, 2H), 3.89-3.88 (m, 1H), 2.78- |
| | 2.76 (m, 1H), 2.67-2.60 (m, 4H), 2.51-2.45 ( m, 1H), 2.45-2.35 (m, 3H), 2.13- |
| | 2.09 (m, 1H), 1.83-1.79 (m, 1H), 1.68 (s, 3H), 1.64-1.55 (m, 2H), 1.42-1.36 (m, |
| | 2H), 1.12-1.03 (m, 2H), 0.92 (s, 3H). |
| 555 | 1H-NMR (400 MHz, DMSO-d6): δ 8.71 (s, 1H), 7.57 (dd, J = 2.80, 8.80 Hz, |
| | 1H), 7.43 (d, J = 2.80 Hz, 1H), 7.32 (t, J = 9.20 Hz, 2H), 4.30 (s, 2H), 4.07 (t, |
| | J = 4.40 Hz, 2H), 2.70 (s, 3H), 2.50-2.45 (m, 3H), 2.33-2.32 (m, 2H), 2.48 (d, J = |
| | 16.00 Hz, 2H), 2.32-2.28 (m, 2H), 2.10 (d, J = 17.60 Hz, 2H), 1.91-1.84 (m, |
| | 1H), 1.65-1.58 (m, 4H), 1.52-1.32 (m, 3H), 0.95 (s, 3H). |
| 556 | 1H-NMR (400 MHz, DMSO-d6): δ 8.56 (s, 1H), 7.56 (dd, J = 2.40, 8.80 Hz, |
| | 1H), 7.54 (d, J = 2.40 Hz, 1H), 7.30 (d, J = 8.80 Hz, 2H), 4.39-4.26 (m, 2H), |
| | 4.07-4.09 (m, 2H), 2.71 (s, 3H), 2.67-2.61 (m, 3H), 2.47 (s, 1H), 2.50 (s, 1H), |
| | 2.50-2.43 (m, 2H), 2.17 (dd, J = 17.20, Hz, 1H), 1.90-1.85 (m, 1H), 1.67-1.65 |
| | (m, 1H), 1.43 (s, 3H), 1.65-1.51 (m, 4H), 1.24 (m, 2H), 0.93 (s, 3H), |
| 557 | 1H-NMR (400 MHz, DMSO-d6): δ 8.58 (s, 1H), 7.55 (dd, J = 2.80, 8.80 Hz, |
| | 1H), 7.42 (d, J = 2.80 Hz, 1H), 7.30 (d, J = 8.80 Hz, 2H), 4.32-4.30 (m, 2H), |
| | 4.09-4.08 (m, 2H), 2.71 (s, 3H), 2.68-2.61 (m, 2H), 2.51-2.50 (m, 2H), 2.49- |
| | 2.44 (m, 1H), 2.33-2.32 (m, 1H), 2.31-2.22 (m, 1H), 2.13 (d, J = 17.20 Hz, 1H), |
| | 1.90-1.89 (m, 1H), 1.67-1.65 (m, 1H), 1.63 (s, 3H), 1.60-1.39 (m, 4H), 1.24- |
| | 1.12 (m, 2H), 0.93 (s, 3H). |
| 558 | 1H-NMR: (400 MHz, DMSO-d6): δ 8.79 (s, 1H), 8.25 (s, 1H), 7.56 (dd, J = 2, |
| | 9 Hz, 1H), 7.45 (d, J = 2.40 Hz, 1H), 7.29-7.31 (m, 1H), 4.29-4.28 (m, 2H), |
| | 4.09-4.08 (m, 2H), 3.36 (s, 3H), 2.86-2.84 (m, 1H), 2.68 (s, 3H), 2.68-2.68 (m, |
| | 1H), 2.67-2.57 (m, 1H), 2.50-2.41 (m, 1H), 2.33-2.32 (m, 2H), 2.12-2.10 (m, |
| | 2H), 1.84-1.82 (m, 4H), 1.60 (s, 3H), 1.30-1.24 (m, 3H), 0.90 (s, 3H), 0.73 (s, 3H). |
| 559 | 1H-NMR: (400 MHz, DMSO-d6): δ 8.77 (s, 1H), 8.24 (s, 1H), 7.57 (dd, J = |
| | 2.80, 8.80 Hz, 1H), 7.44 (d, J = 2.80 Hz, 1H), 7.35-7.30 (m, 1H), 4.33-4.27 (m, |
| | 2H), 4.10-4.06 (m, 2H), 3.801 (s, 3H), 2.733 (s, 3H), 2.68-2.60 (m, 3H), 2.50- |
| | 2.38 (m, 2H), 2.33-2.32 (m, 1H), 2.30-2.17 (m, 1H), 2.07-2.03 (m, 1H), 1.84- |
| | 1.81 (m, 2H), 1.65-1.64 (m, 2H), 1.61 (s, 3H), 1.24 (s, 3H), 0.89 (s, 3H), 0.72- |
| | 0.75 (m, 5H). |
| 560 | 1H-NMR (400 MHz, DMSO-d6): δ 8.66 (s, 1H), 7.56 (dd, J = 2.80, 9.0 Hz, |
| | 1H), 7.43 (d, J = 2.40 Hz, 1H), 7.39 (s, 1H), 7.35 (d, J = 3.60 Hz, 1H), 4.26-4.34 |
| | (m, 2H), 4.04-4.11 (m, 2H), 3.92-3.93 (m, 2H), 2.85-2.94 (m, 2H), 2.60 (s, 6H), |
| | 2.26-2.33 (m, 3H), 1.96-2.14 (m, 1H), 1.66 (m, 6H), 1.25-1.36 (m, 2H), 0.86- |
| | 0.85 (m, 2H), 0.77-0.79 (m, 2H). |
| 561 | 1H-NMR (400 MHz, DMSO-d6): 0 8.79 (s, 1H), 7.57 (dd, J = 2.80, 9.0 Hz, |
| | 1H), 7.44 (d, J = 2.80 Hz, 1H), 7.43 (s, 1H), 7.35 (d, J = 9.20 Hz, 1H), 4.35- |
| | 4.34 (m, 2H), 4.31-4.30 (m, 2H), 3.94 (d, J = 5.60 Hz, 1H), 2.86-2.72 (m, 2H), |
| | 2.75-2.55 (m, 5H), 2.35-2.15 (m, 3H), 1.97 (m, 3H), 1.68-1.65 (m, 5H), 1.34- |
| | 1.21(m, 5H), 0.77-0.66 (m, 2H). |
| 562 | 1H-NMR (400 MHz, DMSO-d6): δ 8.77 (s, 1H), 7.58 (dd, J = 2.80, 9.00 Hz, |
| | 1H), 7.44-7.41 (m, 2H), 7.32 (d, J = 9.20 Hz, 1H), 4.32-4.31 (m, 2H), 4.08-4.07 |
| | (m, 2H), 3.75-3.69 (m, 1H), 3.51 (s, 3H), 3.01-2.90 (m, 2H), 2.73 (s, 3H), 2.51- |
| | 2.49 (m, 4H), 2.32-2.14 (m, 2H), 1.91-1.88 (m, 1H), 1.85-1.61 (m, 3H), 1.58 (s, |
| | 3H), 1.47-1.41(m, 2H), 1.36-1.24- (m, 2H). |
| 563 | 1H-NMR (400 MHz, DMSO-d6): δ 8.74 (s, 1H), 7.57 (dd, J = 2.80, 8.80 Hz, |
| | 1H), 7.43 (d, J = 2.40 Hz, 1H), 7.39 (s, 1H), 7.32 (d, J = 9.20 Hz, 1H), 4.32- |
| | 4.31 (m, 2H), 4.08-4.07 (m, 2H), 3.70-3.65 (m, 1H), 3.46 (s, 3H), 3.01-2.90 (m, |
| | 2H), 2.73 (s, 3H), 2.50- 2.43 (m, 3H), 2.33-2.13 (m, 3H), 1.99-1.80 (m, 1H), |
| | 2.13-1.63 (m, 3H), 1.59 (s, 3H), 1.44-1.43 (m, 2H), 1.41-1.40 (m, 2H). |
| 564 | 1H-NMR (400 MHz, DMSO-d6): δ 13.5 (brs, 1H), 8.80 (s, 1H), 7.59-7.56 (dd, |
| | J = 2.80, 8.80 Hz, 1H), 7.44 (d, J = 2.80 Hz, 1H), 7.40 (s, 1H), 7.33 (d, J = 9.20 |
| | Hz, 1H), 4.37-4.28 (m, 2H), 4.09-4.04 (m, 2H), 2.82-2.69 (m, 4H), 2.65-2.51 |
| | (m, 5H), 2.33-2.24 (m, 1H), 2.03-2.97 (m, 1H), 1.71-1.59 (m, 5H), 1.55-1.45 |
| | (m, 2H), 1.34-1.31 (m, 2H), 1.24-1.20 (s, 2H), 0.81-0.72 (m, 1H), 0.71-0.62 (m, 2H). |
| 565 | 1H-NMR: (400 MHz, DMSO-d6): δ 8.67 (s, 1H), 7.55 (dd, J = 2.80, 8.80 Hz, |
| | 1H), 7.44 (d, J = 2.80 Hz, 1H), 7.30-7.24 (m, 2H), 4.27-4.25 (m, 2H), 4.09-4.01 |
| | (m, 2H), 3.18 (s, 3H), 2.84-2.72 (m, 1H), 2.65-2.35 (m, 4H), 2.62-2.51 (m, 2H), |
| | 2.44-2.23 (m, 2H), 2.20-2.18 (m, 2H), 2.00-1.74 (m, 2H), 1.58 (s, 2H), 1.60- |
| | 1.54 (m, 2H), 1.35-1.34 (m, 2H), 0.90(s, 3H), 0.85 (s, 3H), ), 0.83-0.82 (m, 3H) |
| 566 | 1H-NMR: (400 MHz, DMSO-d6): δ 8.48 (s, 1H), 7.55 (dd, J = 2.80, 8.80 Hz, |
| | 1H), 7.41 (d, J = 2.40 Hz, 1H), 7.26-7.31 (m, 2H), 4.31-4.30 (m, 2H), 4.09-4.07 |
| | (m, 2H), 2.71 (s, 3H), 2.64-2.51 (m, 7H), 2.35-2.12 (m, 5H), 1.81-1.63 (m, 4H), |
| | 1.61 (s, 3H), 0.95 (s, 3H). |
| 567 | 1H-NMR: (400 MHz, DMSO-d6): δ 8.60 (s, 1H), 7.56 (dd, J = 2.80, 9.00 Hz, |
| | 1H), 7.42 (d, J = 2.40 Hz, 1H), 7.32-7.30 (m, 2H), 4.31-4.30 (m, 2H), 4.09-4.08 |
| | (m, 2H), 2.72 (s, 3H), 2.52-2.50 (m, 7H), 2.33-2.12 (m, 5H), 1.81-1.64 (m, 4H), |
| | 1.61 (s, 3H), 0.95 (s, 3H). |
| 568 | 1H-NMR (400 MHz, DMSO-d6): δ 8.74 (brs, 1H), 7.56 (dd, J = 2.80, 9.20 Hz, |
| | 1H), 7.44 (d, J = 2.40 Hz, 1H), 7.37 (brs, 1H), 7.33 (d, J = 8.80 Hz, 1H), 4.36- |
| | 4.27 (m, 2H), 4.11-4.01 (m, 2H), 2.75-2.52 (m, 8H), 2.32-2.15 (m, 7H), 1.95 (t, |
| | J = 12.00 Hz, 1H), 1.64 (s, 3H), 1.38-1.22 (m, 3H), 0.80 (d, J = 3.60 Hz, 1H), |
| | 0.64 (s, 2H). |
| 569 | 1H-NMR (400 MHz, DMSO-d6): δ 8.30 (brs, 1H), 7.54 (dd, J = 2.80, 9.20 Hz, |
| | 1H), 7.40 (d, J = 2.40 Hz, 1H), 7.29 (d, J = 9.20 Hz, 1H), 7.18 (brs, 1H), 4.31- |
| | 4.27 (m, 2H), 4.10 (s, 2H), 2.75-2.52 (m, 8H), 2.32-2.15 (m, 7H), 1.99-1.92 (m, |
| | 1H), 1.65 (s, 3H), 1.40 (d, J = 3.20 Hz, 1H), 1.32-1.22 (m, 2H), 0.80 (d, J = |
| | 3.60 Hz, 1H), 0.62 (s, 2H). |
| 570 | 1H-NMR (400 MHz, DMSO-d6): δ 8.81-8.78 (m, 2H), 7.73 (d, J = 8.40 Hz, |
| | 1H), 7.68-7.65 (m, 2H), 7.50 (d, J = 4.80 Hz, 1H), 4.74 (s, 2H), 2.72 (m, 1H), |
| | 2.67-2.55 (m, 6H), 2.18-2.16 (m, 1H), 1.90 (s, 4H), 1.64-1.62 (m, 2H), 1.52- |
| | 1.50 (m, 3H), 1.28-1.24 (m, 3H). |
| 571 | 1H-NMR (400 MHz, DMSO-d6): δ 8.77 (s, 1H), 7.58 (dd, J = 2.80, 8.80 Hz, |
| | 1H), 7.48-7.44 (m, 3H), 7.38-7.31 (m, 4H), 4.35 (t, J = 10.80 Hz, 2H), 4.08 (t, |
| | J = 4.40 Hz, 2H), 3.12-3.01 (m, 2H), 2.85-2.79 (m, 1H), 2.73 (s, 3H), 2.68-2.66 |
| | (m, 1H), 8 2.57-2.52 (m, 1H), 2.51-2.49 (m, 2H), 2.33-2.23 (m, 3H), 1.94-1.91 |
| | (m, 1H), 1.78-1.61 (m, 5H). 1.64 (s, 3H). |
| 572 | 1H-NMR (400 MHz, DMSO-d6): δ 8.68 (s, 1H), 7.57 (dd, J = 2.40, 9.00 Hz, |
| | 1H), 7.48-7.43 (m, 2H), 7.38-7.30 (m, 5H), 4.37-4.33 (m, 2H), 4.08 (t, J = 4.40 |
| | Hz, 2H), 3.11-3.01 (m, 2H), 2.85-2.81 (m, 1H), 2.72 (s, 3H), 2.68-2.61 (m, 2H), |
| | 2.34-2.23 (m, 5H), 1.94-1.90 (m, 1H), 1.78-1.71 (m, 5H). 1.61 (s, 3H). |
| 573 | 1H-NMR (400 MHz, DMSO-d6): δ 8.83 (s, 1H), 7.58 (dd, J = 2.80, 8.80 Hz, |
| | 1H), 7.45-7.43 (m, 2H), 7.38-7.31 (m, 2H), 7.12-7.07 (m, 1H), 4.33 (t, J = 2.00 |
| | Hz, 2H), 4.08 (t, J = 4.80 Hz, 2H), 3.11-3.00 (m, 2H), 2.95-2.83 (m, 1H), 2.74 |
| | (s, 3H), 2.68-2.63 (m, 1H), 2.55-2.45 (m, 3H), 2.40-2.24 (m, 3H), 1.94-1.91 (m, |
| | 1H), 1.86-1.74 (m, 5H), 1.64 (s, 3H). |
| 574 | 1H-NMR (400 MHz, DMSO-d6): δ 8.84 (s, 1H), 7.58 (dd, J = 2.80, 9.00 Hz, |
| | 1H), 7.44 (t, J = 2.40 Hz, 2H), 7.33 (d, J = 1.20 Hz, 2H), 7.09-7.08 (m, 1H), |
| | 4.33 (t, J = 2.80 Hz, 2H), 4.08 (t, J = 4.40 Hz, 2H), 3.11-3.03 (m, 1H), 3.03- |
| | 2.90 (m, 2H), 2.74 (s, 3H), 2.68-2.67 (m, 2H), 2.34-2.27 (m, 5H), 1.94-1.91 (m, |
| | 1H), 1.85-1.75 (m, 5H). 1.65 (s, 3H). |
| 575 | 1H-NMR (400 MHz, DMSO-d6): δ 13.65 (s, 1H), 8.84 (s, 1H), 8.04 (d, J = |
| | 5.20 Hz, 1H), 7.58 (dd, J = 2.80, 8.80 Hz, 1H), 7.44 (t, J = 2.80 Hz, 2H), 7.33 |
| | (d, J = 8.80 Hz, 1H), 6.88 (dd, J = 1.20, 5.20 Hz, 1H), 6.65 (s, 1H), 4.33 (t, J = |
| | 2.40 Hz, 2H), 4.08 (t, J = 2.00 Hz, 2H), 3.38 (s, 3H), 8 3.08 (d, J = 10.80 Hz, |
| | 1H), 3.00 (d, J = 10.80 Hz, 1H), 2.74 (s, 3H), 2.68-2.67 (m, 1H), 2.50-2.49 (m, |
| | 4H), 2.33-2.22 (m, 3H), 1.93-1.90 (m, 1H), 1.80-1.78 (m, 2H), 1.69-1.59 (m, |
| | 6H) ppm. |
| 576 | 1H-NMR (400 MHz, DMSO-d6): δ 13.67 (s, 1H), 8.69 (s, 1H), 8.04 (d, J = |
| | 5.20 Hz, 1H), 7.56 (dd, J = 16.00, 2.20 Hz, 1H), 7.44-7.43 (m, 1H), 7.37-7.31 |
| | (m, 2H), 6.88 (dd, J = 1.20, 5.60 Hz, 1H), 6.65 (s, 1H), 4.33 (t, J = 6.80 Hz, |
| | 2H), 4.08 (t, J = 4.40 Hz, 2H), 3.81 (s, 3H), 8 3.07 (d, J = 12.00 Hz, 1H), 2.99 |
| | (d, J = 8.00 Hz, 1H), 2.72 (s, 3H), 2.68-2.67 (m, 1H), 2.50-2.48 (m, 4H), 2.34- |
| | 2.21 (m, 3H), 1.92-1.89 (m, 1H), 1.79-1.76 (m, 2H), 1.67-1.58 (m, 6H). |
| 577 | 1H-NMR (400 MHz, DMSO-d6): δ 8.80 (s, 1H), 8.38 (d, J = 0.80 Hz, 1H), |
| | 7.57 (dd, J = 2.80, Hz, 1H), 7.44-7.41 (m, 2H), 7.33 (d, J = 9.20 Hz, 1H), 4.33- |
| | 4.31 (m, 2H), 4.08-4.07 (m, 2H), 3.08-3.07 (m, 3H), 2.73 (s, 3H), 2.66-2.62 (m, |
| | 1H), 2.41-2.32 (m, 5H), 2.32-2.22 (m, 1H), 2.10-2.07 (m, 2H), 1.89 (d, 1H), |
| | 1.72 (s, 6H). |
| 578 | 1H-NMR (400 MHz, DMSO-d6): δ 8.81 (s, 1H), 8.38 (d, J = 0.80 Hz, 1H), |
| | 7.58 (dd, J = 2.40, Hz, 1H), 7.44-7.42 (m, 2H), 7.33 (d, J = 8.8 Hz, 1H), 4.33- |
| | 4.30 (m, 2H), 4.09-4.07 (m, 2H), 3.10-3.00 (m, 3H), 2.66 (s, 3H), 2.53 (d, J = |
| | 1.60 Hz, 1H), 2.49-2.40 (m, 5H), 2.36-2.33 (m, 1H), 2.11-2.09 (m, 2H), 1.94- |
| | 1.89 (m, 1H), 1.77-1.71 (m, 3H), 1.61 (s, 3H). |
| 579 | 1H-NMR (400 MHz, DMSO-d6): δ 12.99 (s, 1H), 8.93 (s, 1H), 7.58 (dd, J = |
| | 2.80, 8.80 Hz, 1H), 7.43-7.40 (m, 2H), 7.34 (d, J = 9.20 Hz, 1H), 4.37-4.28 (m, |
| | 3H), 4.12-4.05 (m, 2H), 3.44 (s, 3H), 2.68-2.61 (m, 6H), 2.61-2.58 (m, 1H), |
| | 2.44-2.39 (m, 2H), 2.33-2.21 (m, 1H), 2.00-1.94 (m, 3H), 1.75-1.67 (m, 5H), |
| | 1.33-1.30 (m, 2H), 0.76-0.73 (m, 3H), |
| 580 | 1H-NMR (400 MHz, DMSO-d6): δ 7.95 (s, 1H), 7.50 (dd, J = 2.80, 9.00 Hz, |
| | 1H), 7.37 (d, J = 2.40 Hz, 1H), 7.25 (d, J = 9.20 Hz, 1H), 7.03 (s, 1H), 4.24 (t, |
| | J = 4.80 Hz, 2H), 4.07 (t, J = 4.40 Hz, 2H), 2.68 (s, 3H), 2.62-2.55 (m, 3H), 2.40 |
| | (d, J = 12.00 Hz, 1H), 2.33-2.31 (m, 1H), 1.99 (s, 1H), 1.73 (t, J = 43.20 Hz, |
| | 1H), 1.55 (s, 3H), 1.52 (s, 1H), 1.24-1.19 (m, 7H), 0.84 (s, 2H), 0.70 (s, 2H), |
| 581 | 1H-NMR (400 MHz, DMSO-d6): δ 8.02 (s, 1H), 7.51 (dd, J = 2.80, 8.80 Hz, |
| | 1H), 7.38 (d, J = 2.00 Hz, 1H), 7.26 (d, J = 8.80 Hz, 1H), 7.07 (s, 1H), 4.25 (t, |
| | J = Hz, 2H), 4.08 (t, J = 10.40 Hz, 2H), 2.72-2.66 (m, 5H), 2.42 (s, 3H), 2.25- |
| | 2.21 (m, 1H), 1.98 (s, 1H), 1.68 (s, 4H), 1.50 (s, 3H), 1.41-1.32 (m, 2H), 1.24- |
| | 1.16 (m, 3H), 0.84 (s, 1H), 0.70 (s, 2H), |
| 582 | 1H-NMR (400 MHz, DMSO-d6): o 8.32 (s, 1H), 7.53 (dd, J = 2.80, 8.80 Hz, |
| | 1H), 7.41 (d, J = 2.80 Hz, 1H), 7.29 (d, J = 8.80 Hz, 1H), 7.20 (s, 1H), 4.27 (t, |
| | J = 4.80 Hz, 2H), 4.06 (t, J = 16.40 Hz, 2H), 2.71-2.54 (m, 5H), 2.44-2.41 (m, |
| | 2H), 2.27-2.20 (m, 1H), 2.12 (s, 1H), 1.96 (t, J = 12.00 Hz, 1H), 1.66 (s, 4H), |
| | 1.50 (s, 3H), 1.39-1.36 (m, 2H), 1.23-1.15 (m, 3H), 0.86 (d, J = 6.00 Hz, 1H), |
| | 0.83 (s, 2H), |
| 583 | 1H-NMR (400 MHz, DMSO-d6): δ 8.71 (s, 1H), 7.57 (dd, J = 2.40, 9.00 Hz, |
| | 1H), 7.44 (d, J = 2.80 Hz, 1H), 7.34 (t, J = 9.20 Hz, 2H), 4.30 (t, J = 4.00 Hz, |
| | 2H), 4.07 (t, J = 6.80 Hz, 2H), 2.71 (s, 3H), 2.68-2.56 (m, 4H), 2.41 (d, J = |
| | 11.20 Hz, 1H), 2.23 (t, J = 11.20 Hz, 1H), 1.92 (t, J = 1.60 Hz, 1H), 1.65 (s, |
| | 4H), 1.55-1.49 (m, 3H), 1.33 (d, J = 6.40 Hz, 2H), 1.24-1.18 (m, 3H), 0.69 (s, |
| | 1H), 0.67 (s, 2H), |
| 584 | 1H-NMR (400 MHz, DMSO-d6): δ 9.02 (s, 2H), 8.57 (m, 1H), 7.56-7.50 (m, |
| | 2H), 7.49-7.31 (m, 2H), 4.32 (m, 2H), 4.09 (m, 2H), 3.15-3.05 (m, 2H), 2.74- |
| | 2.72 (m, 3H), 2.67-2.61 (m, 2H), 2.50-2.49 (m, 1H), 2.35-2.33 (m, 2H), 1.91- |
| | 1.86 (m, 3H), 1.74-1.62 (m, 4H), 1.36-1.24 (m, 5H) |
| 585 | 1H- NMR 400 MHz, DMSO-d6): δ 9.03-9.02 (m, 2H), 8.80 (m, 1H), 7.58 (dd, |
| | J = 2.80, 8.80 Hz, 1H), 7.45-7.44 (m, 2H), 7.33 (d, J = 8.80 Hz, 1H), 4.33 (t, J = |
| | 4.40 Hz, 2H), 4.08 (t, J = 4.40 Hz, 2H), 3.11- 3.03 (m, 2H), 2.75-2.74 (m, 3H), |
| | 2.68-2.67 (m, 1H), 2.67-2.66 (m, 1H), 2.54-2.52 (m, 2H), 2.50-2.33 (m, 3H), 8 |
| | 1.90-1.73 (m, 2H), 1.63-1.60 (m, 4H), 1.35-1.34 (m, 4H) |
| 586 | 1H-NMR (400 MHz, DMSO-d6): δ 8.76 (s, 1H), 7.77-7.73 (m, 1H), 7.58 (dd, |
| | J = 2.80, 8.80 Hz, 1H), 7.50 (dd, J = 2.40, 10.40 Hz, 1H), 7.44 (d, J = 2.40 Hz, |
| | 1H), 7.40 (s, 1H), 7.33 (d, J = 9.20 Hz, 1H), 7.26-7.21 (m, 1H), 4.33 (t, J = 4.40 |
| | Hz, 2H), 4.08 (t, J = 4.40 Hz, 2H), 3.12-3.02 (m, 2H), 2.82-2.79 (m, 1H), 2.73 |
| | (s, 3H), 2.68-2.64 (m, 1H), 2.52-2.49 (m, 3H), 2.35-2.32 (m, 3H), 1.95-1.91 (m, |
| | 1H), 1.81-1.70 (m, 5H), 1.65 (s, 3H). |
| 587 | 1H-NMR: (400 MHz, DMSO-d6): δ 8.37 (s, 1H), 7.71-7.66 (m, 2H), 7.56 (d, |
| | J = 2.4 Hz, 1H), 7.55-7.52 (m, 2H), 7.41 (d, J = 2.80 Hz, 1H), 7.36 (s, 1H), 7.30 |
| | (d, J = 8.80 Hz, 1H), 4.31-4.25 (m, 2H), 4.03-3.99 (m, 2H), 3.50 (d, J = 14.3 |
| | Hz, 1H), 3.19 (d, J = 9.60 Hz, 1H), 2.52-2.51 (m, 3H), 2.34-2.31 (m, 4H), 2.14- |
| | 1.98 (m, 1H), 1.74-1.71 (m, 2H), 1.67-1.48 (m, 3H), 1.45-1.35 (m, 5H), 1.34- |
| | 1.26 (m, 2H), |
| 588 | 1H-NMR (400 MHz, DMSO-d6): δ 8.35 (s, 1H), 7.67-7.66 (m, 2H), 7.56 (d, J = |
| | 2.8 Hz, 1H), 7.54-7.52 (m, 2H), 7.41 (d, J = 2.40 Hz, 1H), 7.34 (s, 1H), 7.29 (d, |
| | J = 9.2 Hz, 1H), 4.31-4.24 (m, 2H), 4.24-4.02 (m, 2H), 3.69 (d, J = 8.40 Hz, |
| | 1H), 3.08 (d, J = 10.8 Hz, 1H), 2.66-2.50 (m, 3H), 2.37-2.33 (m, 4H), 2.32-2.31 |
| | (m, 1H), 1.98-1.77 (m, 2H), 1.74-1.51 (m, 3H), 1.71-1.48 (m, 7H). |
| 589 | 1H-NMR (400 MHz, DMSO-d6): δ 8.21 (s, 1H), 7.69-7.64 (m, 2H), 7.58-7.52 |
| | (m, 3H), 7.43-7.40 (d, J = 2.40 Hz, 1H), 7.35 (s, 1H), 7.32-7.28 (d, J = 8.80 Hz, |
| | 1H), 4.31-4.27 (m, 2H), 4.03-3.98 (m, 2H), 3.65-3.63 (m, 1H), 3.32-3.00 (m, |
| | 1H), 2.52-2.51 (m, 3H), 2.35-2.33 (m, 4H), 2.21-2.15 (m, 2H), 1.78-1.66 (m, |
| | 5H), 1.66-1.52 (m, 6H). |
| 590 | 1H-NMR (400 MHz, DMSO-d6): δ 8.21 (s, 1H), 7.66-7.58 (m, 2H), 7.57 (d, J = |
| | 2.8 Hz, 1H), 7.55 (d, J = 2.4 Hz, 1H), 7.52-7.51 (m, 1H), 7.41 (d, J = 2.8 Hz, |
| | 1H) 7.34 (s, 1H), 7.30 (d, J = 8.8 Hz, 1H), 4.31-4.03 (m, 2H), 4.01-3.98 (m, |
| | 2H), 3.65-3.62 (m, 1H), 3.32-3.00 (m, 1H), 2.52-2.51 (m, 3H), 2.33-2.21 (m, |
| | 3H), 2.19-2.15 (m, 2H), 1.85-1.55 (m, 6H), 1.52 (s, 3H), 1.48-1.35 (m, 3H). |
| 591 | 1H-NMR (400 MHz, DMSO-d6): δ 13.5 (brs, 1H), 8.80 (s, 1H), 7.59-7.56 (dd, |
| | J = 2.80, 8.80 Hz, 1H), 7.44 (d, J = 2.80 Hz, 1H), 7.40 (s, 1H), 7.33 (d, J = 9.2 |
| | Hz, 1H), 4.37-4.28 (m, 2H), 4.09-4.04 (m, 2H), 2.82-2.69 (m, 4H), 2.65-2.51 |
| | (m, 5H), 2.33-2.24 (m, 1H), 2.03-2.97 (m, 1H), 1.71-1.59 (m, 5H), 1.55-1.45 |
| | (m, 2H), 1.34-1.31 (m, 2H), 1.24-1.20 (s, 2H), 0.81-0.72 (m, 1H), 0.71-0.62 (m, 2H). |
| 592 | 1H-NMR (400 MHz, DMSO-d6): δ 9.02 (s, 1H), 8.86 (d, J = 4.80 Hz, 1H), |
| | 7.75-7.74 (m, 1H), 7.70-7.67 (m, 2H), 7.58 (d, J = 4.80 Hz, 1H), 4.74 (s, 2H), |
| | 4.45 (s, 1H), 3.47 (s, 3H), 2.96-2.86 (m, 3H), 2.58-2.52 (m, 2H), 2.28-2.20 (m, |
| | 2H), 1.99 (m, 3H), 1.90 (s, 3H), 1.82-1.70 (m, 3H), 1.23 (s, 2H). |
| 593 | 1H-NMR (400 MHz, DMSO-d6): δ 8.85 (m, 1H), 7.99-7.94 (m, 2H), 7.58 (dd, |
| | J = 2.80, 8.80 Hz, 1H), 7.45-7.44 (m, 2H), 7.37-7.32 (m, 2H), 4.34-4.32 (m, |
| | 2H), 4.09-4.08 (m, 2H), 3.17-3.08 (m, 3H), 2.74 (s, 3H), 2.68-2.67 (m, 1H), |
| | 2.67-2.66 (m, 3H), 2.50-2.49 (m, 1H), 2.33 (d, J = 1.60 Hz, 2H), 1.83-1.78 (m, |
| | 3H), 1.71-1.65 (m, 4H), 1.26-1.24 (m, 2H), |
| 594 | 1H-NMR (400 MHz, DMSO-d6): δ 8.77 (s, 1H), 7.99-7.94 (m, 2H), 7.58 (dd, |
| | J = 2.40, 9.00 Hz, 1H), 7.43-7.33 (m, 4H), 4.33 (m, 2H), 4.09-4.08 (m, 2H), |
| | 3.13-3.09 (m, 3H), 2.73 (s, 3H), 2.68-2.64 (m, 3H), 2.53-2.50 (m, 1H), 2.48 (d, |
| | J = 2.00 Hz, 2H), 2.38-2.33 (m, 1H), 2.27-2.12 (m, 2H), 1.76-1.60 (m, 5H), |
| | 1.25-1.23 (m, 2H) |
| 595 | 1H-NMR (400 MHz, DMSO-d6): δ 8.21 (s, 1H), 7.53 (dd, J = 2.00, 9.00 Hz, |
| | 1H), 7.38 (d, J = 2 Hz, 1H), 7.28-7.22 (m, 3H), 7.15 (s, 1H), 7.00-6.96 (m, 1H), |
| | 4.31-4.27 (m, 2H), 4.08-4.07 (m, 2H), 2.84-2.79 (m, 2H), 2.67 (s, 3H), 2.57- |
| | 2.52 (m, 2H), 2.43-2.35 (m, 5H), 2.23-2.16 (m, 1H), 1.94 (s, 3H), 1.65-1.58 (m, 6H). |
| 596 | 1H-NMR (400 MHz, DMSO-d6): δ 8.33 (s, 1H), 7.55 (dd, J = 2.40, 9.00 Hz, |
| | 1H), 7.38 (d, J = 2.40 Hz, 1H), 7.26-7.20 (m, 4H), 7.02-6.99 (m, 1H), 4.33- |
| | 4.29 (m, 2H), 4.10-4.09 (m, 2H), 2.87-2.79 (m, 2H), 2.68 (s, 3H), 2.60-2.52 (m, |
| | 2H), 2.45-2.39 (m, 4H), 2.21-2.20 (m, 1H), 1.98-1.89 (m, 4H), 1.64-1.61 (s, 6H). |
| 597 | 1H-NMR (400 MHz, DMSO-d6): δ 8.43 (m, 1H), 7.54 (dd, J = 2.40, 8.80 Hz, |
| | 1H), 7.41 (m, 1H), 7.29-7.23 (m, 2H), 4.45-4.44 (m, 1H), 4.29-4.28 (m, 2H), |
| | 4.11-4.06 (m, 2H), 2.97-2.84 (m, 3H), 2.75 (s, 3H), 2.68-0.57 (m, 3H), 1.95- |
| | 1.89 (m, 3H), 1.72-1.60 (m, 5H), 1.24-1.23 (m, 2H), 1.08-1.06 (m, 1H), 1.05- |
| | 1.03 (m, 1H) |
| 598 | 1H-NMR (400 MHz, DMSO-d6): δ 8.50 (m, 1H), 7.55 (dd, J = 2.80, 9.00 Hz, |
| | 1H), 7.42 (d, J = 2.40 Hz, 1H), 7.28 (d, J = 8.80 Hz, 2H), 4.46-4.44 (m, 1H), |
| | 4.43-4.28 (m, 2H), 4.07-4.04 (m, 2H), 2.99-2.85 (m, 3H), 2.71 (s, 3H), 2.58- |
| | 2.50 (m, 3H), 1.97-1.83 (m, 3H), 1.72-1.66 (m, 5H), 1.25-1.23 (m, 2H), 1.09- |
| | 1.08 (m, 1H), 1.01- 0.98 (m, 1H) |
| 599 | 1H-NMR (400 MHz, DMSO-d6): δ 8.74 (s, 1H), 7.57 (dd, J = 2.80, 9.00 Hz, |
| | 1H), 7.45 (d, J = 2.80 Hz, 1H), 7.38 (s, 1H), 7.31 (d, J = 8.80 Hz, 1H), 4.31 (t, |
| | J = 5.20 Hz, 2H), 4.07 (t, J = 3.20 Hz, 2H), 2.73 (s, 3H), 2.56-2.42 (m, 6H), 2.43- |
| | 2.09 (m, 4H), 2.09 (s, 3H), 2.00-1.90 (m, 1H), 1.82-1.78 (m, 1H), 1.68-1.61 (m, |
| | 1H), 1.60 (s, 3H), 0.89 (s, 3H). |
| 600 | 1H-NMR (400 MHz, DMSO-d6): δ 8.73 (s, 1H), 7.57 (dd, J = 2.80, 8.80 Hz, |
| | 1H), 7.44 (d, J = 2.80 Hz, 1H), 7.38 (s, 1H), 7.30 (d, J = 8.80 Hz, 1H), 4.31 (t, |
| | J = 4.80 Hz, 2H), 4.07 (t, J = 4.80 Hz, 2H), 2.73 (s, 3H), 2.56-2.25 (m, 6H), 2.15- |
| | 2.09 (m, 4H), 2.09 (s, 3H), 2.00-1.90 (m, 1H), 1.82-1.78 (m, 1H), 1.68-1.61 (m, |
| | 1H), 1.60 (s, 3H), 0.89 (s, 3H). |
| 601 | 1H-NMR (400 MHz, DMSO-d6): δ 8.95-8.82 (m, 2H), 7.75-7.73 (m, 1H), |
| | 7.68-7.66 (m, 2H), 7.54 (s, 1H), 4.74 (s, 2H), 4.45 (s, 1H), 3.32 (m, 3H), 2.86- |
| | 2.80 (m, 2H), 2.70-2.67 (m, 1H), 2.57 (m, 2H), 2.46 (m, 2H), 1.99 (m, 3H), 1.95 |
| | (s, 3H), 1.73-1.63 (m, 3H), 1.24 (s, 2H). |
| 602 | 1H-NMR (400 MHz, DMSO-d6): δ 8.85 (s, 1H), 7.59-7.57 (m, 1H), 7.45-7.42 |
| | (m, 2H), 7.35-7.33 (d, J = 8.80 Hz, 1H), 4.38-4.31 (m, 2H), 4.13-4.08 (m, 2H), |
| | 3.01-2.96 (m, 2H), 2.72 (s, 3H), 2.67-2.62 (m, 1H), 2.33-2.32 (m, 4H), 2.03- |
| | 1.99 (m, 1H), 1.91-1.80 (m, 2H), 1.64 (s, 3H), 1.44-1.43 (m, 2H), 1.34-1.32 (m, |
| | 2H), 1.24-1.19 (m, 4H) |
| 603 | 1H-NMR (400 MHz, DMSO-d6): δ 8.85 (s, 1H), 7.59-7.57 (m, 1H), 7.45-7.42 |
| | (m, 2H), 7.35-7.33 (d, J = 8.80 Hz, 1H), 4.38-4.30 (m, 2H), 4.11-4.02 (m, 2H), |
| | 3.01-2.93 (m, 2H), 2.71 (s, 3H), 2.70-2.62 (m, 4H), 2.33-2.29 (m, 4H), 2.10- |
| | 1.99 (m, 2H), 1.81-1.79 (m, 2H), 1.64 (s, 3H), 1.44-1.41 (m, 2H), 1.34-1.31 (m, 2H). |
| 604 | 1H-NMR (400 MHz, DMSO-d6): δ 8.59 (s, 1H), 7.56 (dd, J = 2.40, 9.00 Hz, |
| | 1H), 7.43 (d, J = 2.40 Hz, 1H), 7.30-7.32 (m, 2H), 4.59 (d, J = 9.20 Hz, 1H), |
| | 4.31 (d, J = 2.80 Hz, 2H), 4.07-4.06 (m, 2H), 3.10-3.06 (m, 1H), 2.98-2.96 (m, |
| | 1H), 2.79-2.64 (m, 5H), 2.59-2.56 (m, 1H), 2.21-2.03 (m, 2H), 1.95-1.89 (m, |
| | 1H), 1.88-1.79 (m, 1H), 1.78-1.68 (m, 3H), 1.65 (s, 3H), 1.58-1.45 (m, 2H), |
| | 1.38-1.27 (m, 2H), 1.26-1.21 (m, 1H), 1.18-1.11 (m, 2H). |
| 605 | 1H-NMR (400 MHz, DMSO-d6): δ 8.66 (s, 1H), 7.56 (dd, J = 2.40, 9.00 Hz, |
| | 1H), 7.43 (d, J = 2.80 Hz, 1H), 7.32-7.30 (m, 2H), ), 4.59 (d, J = 9.60 Hz, 1H), |
| | 4.33-4.28 (m, 2H), 4.07-4.06 (m, 2H), 2.96 (s, 1H), 2.95-2.93 (m, 1H), 2.83- |
| | 2.65 (m, 6H), 2.17-1.99 (m, 2H), 1.96-1.82 (m, 2H), 1.81-1.68 (m, 3H), 1.65 (s, |
| | 3H), 1.58-1.45 (m, 2H), 1.42-1.29 (m, 2H), 1.28-1.22 (m, 1H), 1.18-1.11 (m, 2H). |
| 606 | 1H-NMR (400 MHz, DMSO-d6): δ 8.66 (s, 1H), 7.56 (dd, J = 2.80, 9.00 Hz, |
| | 1H), 7.43 (d, J = 2.40 Hz, 1H), 7.34-7.31 (m, 2H), 4.59 (d, J = 9.60 Hz, 1H), |
| | 4.32 (d, J = 3.60 Hz, 2H), 4.07-4.06 (m, 2H), 3.10-3.05 (m, 1H), 2.98 (s, 1H), |
| | 2.78-2.65 (m, 5H), 2.58-2.54 (m, 1H), 2.21-2.03 (m, 2H), 1.97-1.88 (m, 1H), |
| | 1.87-1.71 (m, 3H), 1.65 (s, 3H), 1.71-1.66 (m, 1H), 1.59-1.45 (m, 2H), 1.38- |
| | 1.27 (m, 2H), 1.26-1.21 (m, 1H), 1.18-1.11 (m, 2H). |
| 607 | 1H-NMR (400 MHz, DMSO-d6): δ 8.55 (s, 1H), 7.55 (dd, J = 2.80, 9.00 Hz, |
| | 1H), 7.43 (d, J = 2.40 Hz, 1H), 7.31 (d, J = 8.80 Hz, 2H), 4.60-4.58 (m, 1H), |
| | 4.33-4.29 (m, 2H), 4.07-4.06 (m, 2H), 2.99-2.97 (m, 1H), 2.95-2.93 (m, 1H), |
| | 2.68 (s, 3H), 2.83-2.69 (m, 3H), 2.18-1.99 (m, 2H), 1.65 (s, 3H), 1.98-1.68 (m, |
| | 5H), 1.59-1.45 (m, 2H), 1.43-1.28 (m, 2H), 1.29-1.21 (m, 1H), 1.18-1.11 (m, 2H). |
| 608 | 1H-NMR (400 MHz, DMSO-d6): δ 8.76 (s, 1H), 7.73-7.64 (m, 3H), 7.43 (s, |
| | 1H), 4.84 (d, J = 18.00 Hz, 1H), 4.69 (d, J = 18.00 Hz, 1H), 2.68 (s, 3H), 2.54- |
| | 2.49 (m, 4H), 2.41-2.31 (m, 3H), 2.10 (d, J = 17.20 Hz, 1H), 1.93-1.83 (m, 4H), |
| | 1.72-1.68 (m, 1H), 1.55-1.43 (m, 4H), 1.38-1.36 (m, 1H), 1.34 (s, 4H). |
| 609 | 1H-NMR (400 MHz, DMSO-d6): δ 8.30 (s, 1H), 7.53 (dd, J = 2.40, 8.80 Hz, |
| | 1H), 7.40 (d, J = 2.40 Hz, 1H), 7.29 (d, J = 2.40 Hz, 1H), 7.18 (s, 1H), 5.95 (td, |
| | J = 4.80, 56.80 Hz, 1H), 4.31-4.25 (m, 2H), 4.04-4.12 (m, 2H), 2.89-2.53 (m, |
| | 6H), 2.36-2.25 (m, 1H), 2.15 (t, J = 10.40 Hz, 2H), 1.98 (t, J = 11.60 Hz, 2H), |
| | 1.82-1.67 (m, 2H), 1.64 (s, 3H), 1.47-1.15 (m, 5H), 0.85-0.68 (m, 1H), 0.65- |
| | 0.58 (m, 2H). |
| 610 | 1H-NMR (400 MHz, DMSO-d6): δ 8.53 (s, 1H), 7.55 (dd, J = 2.40, 8.80 Hz, |
| | 1H), 7.42 (d, J = 2.40 Hz, 1H), 7.31 (d, J = 9.20 Hz, 2H), 5.95 (td, J = 4.80, |
| | 56.80 Hz, 1H), 4.33-4.28 (m, 2H), 4.11-4.02 (m, 2H), 2.83-2.51 (m, 6H), 2.38- |
| | 2.23 (m, 3H), 1.97 (t, J = 12.00 Hz, 2H), 1.71-1.65 (s, 5H), 1.45-1.14 (m, 5H), |
| | 0.85-0.68 (m, 1H), 0.65-0.58 (m, 2H). |
| 611 | 1H-NMR: (400 MHz, DMSO-d6): δ 8.66 (s, 1H), 8.40 (m, 1H), 7.56 (dd, J = |
| | 2.80 Hz, 8.8 Hz, 1H), 7.42 (d, J = 2.8 Hz, 1H), 7.34-7.29 (m, 2H), 7.28-7.27 (m, |
| | 2H), 7.01-6.96 (m, 1H), 4.34-4.29 (m, 3H), 4.08-4.05 (m, 2H), 2.77-2.51 (m, |
| | 6H), 2.51-2.48 (m, 1H), 2.50-2.42 (m, 2H), 2.38-2.30 (m, 1H), 2.02-. 1.90 (m, |
| | 3H), 1.63-1.61 (m, 5H), 1.33 (d, J = 2.80 Hz, 2H), 1.15-1.14 (m, 3H), 0.80 (d, |
| | J = 6.80 Hz, 1H). |
| 612 | 1H-NMR (400 MHz, DMSO-d6): δ 8.43 (s, 1H), 7.71 (d, J = 8.40 Hz, 1H), |
| | 7.63-7.72 (m, 2H), 7.31 (s, 1H), 4.78 (s, 2H), 2.68 (s, 1H), 2.67-2.61 (m, 4H), |
| | 2.49-2.35 (m, 3H), 2.25-2.22 (m, 1H), 2.12 (d, J = 17.20 Hz, 1H), 1.94 (s, 3H), |
| | 1.84-1.81 (m, 1H), 1.69-1.55 (m, 2H), 1.45-1.40 (m, 3H), 1.23 (s, 1H), 1.16- |
| | 1.15 (m, 2H), 1.11 (s, 3H), |
| 613 | 1H-NMR: (400 MHz, DMSO-d6): δ 8.77 (s, 1H), 7.58 (dd, J = 2.4 Hz, 8.8 Hz, |
| | 1H), 7.44 (d, J = 2.80 Hz, 1H), 7.38 (s, 1H), 7.33 (d, J = 2.80 Hz, 1H), 7.29-7.21 |
| | (m, 2H), 7.01-6.96 (m, 1H), 4.36-4.30 (m, 3H), 4.09-4.04 (m, 2H), 2.84-2.71 |
| | (m, 6H), 2.66-2.62 (m, 1H), 2.50-2.42 (m, 2H), 2.38-2.27 (m, 1H), 2.02-1.09 |
| | (m, 3H), 1.65-1.62 (m, 5H), 1.35-1.32 (m, 2H), 0.81-0.74 (m, 1H), 0.69-0.66 |
| | (m, 2H), |
| 614 | 1H-NMR (400 MHz, DMSO-d6): δ 8.73 (s, 1H), 7.73-7.64 (m, 3H), 7.42 (s, |
| | 1H), 4.76 (s, 2H), 2.75 (s, 1H), 2.68 (s, 3H), 2.55-2.54 (m, 1H), 2.42-2.26 (m, |
| | 5H), 2.10 (d, J = 17.20 Hz, 1H), 1.93-1.83 (m, 4H), 1.71-1.66 (m, 1H), 1.55- |
| | 1.41 (m, 1H), 1.43-1.41 (m, 3H), 1.38-1.36 (m, 2H), 1.34 (s, 3H). |
| 615 | 1H-NMR (400 MHz, DMSO-d6): δ 8.78 (s, 1H), 7.73 (d, J = 8.40 Hz, 1H), |
| | 7.67-7.65 (m, 2H), 7.44 (s, 1H), 4.85-4.66 (m, 2H), 2.71 (s, 3H), 2.68-2.50 (m, |
| | 3H), 2.38-2.31 (m, 4H), 2.10 (d, J = 10.00 Hz, 1H), 1.93 (s, 3H), 1.85-1.80 (m, |
| | 1H), 1.72-1.65 (m, 1H), 1.45-1.15 (m, 4H), 1.12-1.10 (m, 1H), 1.08 (d, J = 8.40 |
| | Hz, 1H), 1.06 (s, 3H), |
| 616 | 1H-NMR (400 MHz, DMSO-d6): δ 8.63 (s, 1H), 7.56 (dd, J = 2.40, 9.00 Hz, |
| | 1H), 7.43 (d, J = 2.80 Hz, 1H), 7.32 (d, J = 8.80 Hz, 2H), 5.95 (td, J = 4.80, |
| | 56.80 Hz, 1H), 4.35-4.29 (m, 2H), 4.08-4.04 (m, 2H), 2.82-2.52 (m, 6H), 2.33- |
| | 2.16 (m, 3H), 2.00-1.89 (m, 2H), 1.71-1.69 (m, 2H), 1.65 (s, 3H), 1.35-1.30 (m, |
| | 2H), 1.16 (s, 3H), 0.85-0.68 (m, 1H), 0.65-0.58 (m, 2H).. |
| 617 | 1H-NMR (400 MHz, DMSO-d6): δ 8.80 (s, 1H), 7.57 (dd, J = 2.80, 8.80 Hz, |
| | 1H), 7.44 (d, J = 2.80 Hz, 1H), 7.40-7.32 (m, 2H), 5.95 (td, J = 4.80, 56.80 Hz, |
| | 1H), 4.33-4.28 (m, 2H), 4.11-4.04 (m, 2H), 2.89-2.50 (m, 6H), 2.29-2.22 (m, |
| | 1H), 2.17 (t, J = 10.00 Hz, 2H), 1.97 (t, J = 5.60 Hz, 2H), 1.69-1.63 (m, 2H), |
| | 1.64 (s, 3H), 1.48-1.15 (m, 5H), 0.85-0.68 (m, 1H), 0.65-0.58 (m, 2H). |
| 618 | 1H-NMR (400 MHz, DMSO-d6): δ 8.71 (s, 1H), 7.57 (dd, J = 2.80, 9.00 Hz, |
| | 1H), 7.44 (d, J = 2.80 Hz, 1H), 7.36 (s, 1H), 7.32 (d, J = 8.80 Hz, 1H), 4.30- |
| | 4.34 (m, 2H), 4.05-4.07 (m, 2H), 2.74-2.71 (m, 4H), 2.68-2.66 (m, 4H), 2.50- |
| | 2.44 (m, 3H), 2.28-2.25 (m, 3H), 1.89-1.81(m, 3H), 1.65 (s, 3H), 1.51 (d, J = |
| | 12.80 Hz, 1H), 1.32-1.30 (m, 1H), 0.83-0.80 (m, 1H), 0.69-0.68 (m, 2H). |
| 619 | 1H-NMR (400 MHz, DMSO-d6): δ 8.75 (s, 1H), 7.57 (dd, J = 2.80, 9.00 Hz, |
| | 1H), 7.44 (d, J = 2.40 Hz, 1H), 7.38 (s, 1H), 7.33 (d, J = 8.80 Hz, 1H), 4.36- |
| | 4.29 (m, 2H), 4.07-4.05 (m, 2H), 2.72-2.67 (m, 4H), 2.67-2.57 (m, 4H), 2.46- |
| | 2.41 (m, 3H), 2.28-2.18 (m, 3H), 1.89-1.83 (m, 3H), 1.65 (s, 3H), 1.51 (d, J = |
| | 12.80 Hz, 1H), 1.31-1.28 (m, 1H), 0.83-0.82 (m, 1H), 0.69-0.67 (m, 2H). |
| 620 | 1H-NMR (400 MHz, DMSO-d6): δ 13.59 (s, 1H), 8.77 (s, 1H), 7.57 (dd, J = |
| | 2.80, 8.80 Hz, 1H), 7.44 (d, J = 2.80 Hz, 1H), 7.38 (s, 1H), 7.31 (d, J = 9.20 Hz, |
| | 1H), 4.32 (t, J = 4.40 Hz, 2H), 4.08 (t, J = 5.20 Hz, 2H), 2.91-2.88 (m, 2H), |
| | 2.74 (s, 3H), 2.59-2.51 (m, 2H), 2.42-2.37 (m, 3H), 2.33-2.19 (m, 9H), 1.87- |
| | 1.74 (m, 4H), 1.59-1.51 (m, 4H), 1.40-1.37 (m, 1H), 1.23 (s, 1H). |
| 621 | 1H-NMR (400 MHz, DMSO-d6): δ 13.63 (s, 1H), 8.75 (s, 1H), 7.57 (dd, J = |
| | 2.80, 8.80 Hz, 1H), 7.44 (d, J = 2.80 Hz, 1H), 7.37 (s, 1H), 7.31 (d, J = 8.80 Hz, |
| | 1H), 4.32 (t, J = 4.40 Hz, 2H), 4.08 (t, J = 5.60 Hz, 2H), 2.91-2.88 (m, 2H), |
| | 2.73 (s, 3H), 2.59-2.51 (m, 2H), 2.42-2.40 (m, 3H), 2.27 (s, 9H), 1.85-1.72 (m, |
| | 4H), 1.59 (s, 4H), 1.57-1.51 (m, 1H), 1.40-1.37 (m, 1H), |
| 622 | 1H-NMR 400 MHz, CD3OD): δ 8.84 (s, 1H), 8.51 (brs, 1H), 7.77-7.66 (brs, |
| | 1H), 7.59 (dd, J = 2.80, 8.80 Hz, 1H), 7.46 (d, J = 2.80 Hz, 2H), 7.33 (d, J = |
| | 9.20 Hz, 1H), 4.66-4.32 (m, 3H), 4.10-3.94 (m, 2H), 3.50-3.48 (m, 1H), 3.43- |
| | 3.39 (m, 2H), 3.32-3.28 (m, 1H), 3.14-2.91 (m, 2H), 2.89-2.79 (m, 4H), 2.60- |
| | 2.51 (m, 2H), 2.42-2.40 (m, 2H), 2.21-1.59 (m, 6H), 1.18 (d, J = 4.00 Hz, 2H). |
| 623 | 1H-NMR 400 MHz, DMSO-d6): δ 11.02 (s, 1H), 8.86 (s, 1H), 8.58 (brs, 1H), |
| | 7.77 (brs, 1H), 7.60 (dd, J = 4.00, 8.00 Hz, 1H), 7.48 (s, 1H), 7.46 (d, J = 4.00 |
| | Hz, 1H), 7.33 (d, J = 8.00 Hz, 1H), 4.76-4.83 (m, 1H), 4.38-4.25 (m, 2H), 4.12- |
| | 4.04 (m, 2H), 3.55-3.47 (m, 1H), 3.43-3.42 (m, 1H), 3.39 (s, 1H), 3.29-3.25 (m, |
| | 1H), 3.17-3.05 (m, 3H), 2.76 (s, 3H), 2.65-2.60 (m, 2H), 2.07-1.60 (m, 8H), |
| | 1.18 (d, J = 4.00 Hz, 2H). |

### 11. Biological Assessment

### Biochemical Competitive Binding Assay

The potencies of compounds binding to human eIF4E protein were measured using a 384-well time-resolved fluorescence resonance energy transfer (TR-FRET) competition assay. The assay was performed in assay buffer containing 50 mM HTEPES pH 7.5, 100 mM KCl, 0.02% Tween-20, and 0.1 mg/mL bovine serum albumin (BSA). The reaction volume was 10 μL and each reaction contained 4 nM recombinant 6×HIS-tagged human EIF4E protein (Novus, NBP-45314), 5 nM EDA-m7GDP-ATTO-647N (Jena Bioscience, NU-827-647N), 2.5 nM Europium conjugated Anti-6×HIS antibody (PerkinElmer, AD0402) and varying concentrations of compound. The final DMSO concentration was 100.

Compounds were prepared using 11-point, 4-fold serial dilutions in DMSO and 100 nL of diluted compounds were transferred to 384-well assay-ready plates. Recombinant human EIF4E protein at 2× final concentration (8 nM) was pre-incubated with Eu-anti-6×HIS antibody at 2× final concentration (5 nM) for 5 minutes, then 5 μL of the protein solution was added to assay-ready plates. The protein/compound mix was incubated for 15 minutes, after which 5 μL of a solution containing EDA-m7GDP-ATTO-647N probe at 2× final concentration (10 nM) was added. After a subsequent 15-minute incubation, time-resolved fluorescence of assay plates was measured using a Clariostar Plus microplate reader (BMG Labtech) and TR-FRET values were calculated by taking a ratio of the 665 to 620 wavelength signals. After normalization to the average of DMSO control and maximum inhibition control wells, concentration-response data were plotted and standard 4-parameter curve fitting (PEI Signals) was utilized to determine IC₅₀ values. Results of the competitive binding assay are summarized in Table E3.

TABLE E3

| Cmpd | hEIF4E |
|---|---|
| No. | TR-FRET IC₅₀ |
| 1 | A |
| 2 | A |
| 3 | A |
| 4 | A |
| 5 | B |
| 6 | B |
| 7 | B |
| 8 | A |
| 9 | A |
| 10 | A |
| 11 | A |
| 12 | A |
| 13 | A |
| 14 | A |
| 15 | C |
| 16 | A |
| 17 | A |
| 18 | A |
| 19 | A |
| 20 | A |
| 21 | A |
| 22 | A |
| 23 | A |
| 24 | A |
| 25 | B |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | B |
| 39 | A |
| 40 | A |
| 41 | A |
| 42 | A |
| 43 | A |
| 44 | A |
| 45 | A |
| 46 | A |
| 47 | A |
| 48 | A |
| 49 | B |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | C |
| 76 | A |
| 77 | A |
| 78 | B |
| 79 | A |
| 80 | A |
| 81 | A |
| 82 | A |
| 83 | A |
| 84 | A |
| 85 | B |
| 86 | A |
| 87 | A |
| 88 | A |
| 89 | A |
| 90 | A |
| 91 | A |
| 92 | B |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | A |
| 116 | A |
| 117 | A |
| 118 | C |
| 119 | A |
| 120 | A |
| 121 | A |
| 122 | A |
| 123 | C |
| 124 | A |
| 125 | A |
| 126 | A |
| 127 | A |
| 128 | A |
| 129 | A |
| 130 | A |
| 131 | A |
| 132 | A |
| 133 | B |
| 134 | A |
| 135 | A |
| 136 | A |
| 137 | A |
| 138 | B |
| 139 | A |
| 140 | A |
| 141 | A |
| 142 | B |
| 143 | A |
| 144 | C |
| 145 | C |
| 146 | A |
| 147 | A |
| 148 | A |
| 149 | A |
| 150 | C |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | C |
| 234 | C |
| 235 | A |
| 236 | A |
| 237 | C |
| 238 | C |
| 239 | A |
| 240 | A |
| 241 | A |
| 242 | A |
| 243 | A |
| 244 | B |
| 245 | A |
| 246 | A |
| 247 | A |
| 248 | A |
| 249 | A |
| 250 | A |
| 251 | A |
| 252 | A |
| 253 | B |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | B |
| 266 | C |
| 267 | A |
| 268 | C |
| 269 | A |
| 270 | A |
| 271 | A |
| 272 | C |
| 273 | C |
| 274 | B |
| 275 | A |
| 276 | A |
| 277 | C |
| 278 | A |
| 279 | A |
| 280 | B |
| 281 | B |
| 282 | A |
| 283 | A |
| 284 | A |
| 285 | A |
| 286 | A |
| 287 | A |
| 288 | A |
| 289 | A |
| 290 | A |
| 291 | A |
| 292 | A |
| 293 | A |
| 294 | A |
| 295 | A |
| 296 | C |
| 297 | C |
| 298 | A |
| 299 | A |
| 300 | A |
| 301 | A |
| 302 | A |
| 303 | A |
| 304 | A |
| 305 | A |
| 306 | C |
| 307 | B |
| 308 | B |
| 309 | B |
| 310 | A |
| 311 | A |
| 312 | A |
| 313 | A |
| 314 | A |
| 315 | A |
| 316 | A |
| 317 | A |
| 318 | A |
| 319 | B |
| 320 | B |
| 321 | A |
| 322 | A |
| 323 | A |
| 324 | A |
| 325 | A |
| 326 | A |
| 327 | A |
| 328 | A |
| 329 | A |
| 330 | A |
| 331 | B |
| 332 | B |
| 333 | A |
| 334 | A |
| 335 | A |
| 336 | A |
| 337 | A |
| 338 | A |
| 339 | C |
| 340 | C |
| 341 | C |
| 342 | A |
| 343 | A |
| 344 | A |
| 345 | A |
| 346 | A |
| 347 | A |
| 348 | A |
| 349 | A |
| 350 | C |
| 351 | A |
| 352 | A |
| 353 | C |
| 354 | A |
| 355 | A |
| 356 | A |
| 357 | A |
| 358 | C |
| 359 | A |
| 360 | A |
| 361 | A |
| 362 | A |
| 363 | A |
| 364 | A |
| 365 | A |
| 366 | A |
| 367 | A |
| 368 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | A |
| 387 | A |
| 388 | A |
| 389 | A |
| 390 | A |
| 391 | A |
| 392 | A |
| 393 | A |
| 394 | A |
| 395 | A |
| 396 | A |
| 397 | B |
| 398 | B |
| 399 | A |
| 400 | A |
| 401 | A |
| 402 | A |
| 403 | A |
| 404 | A |
| 405 | A |
| 406 | A |
| 407 | A |
| 408 | A |
| 409 | A |
| 410 | A |
| 411 | A |
| 412 | A |
| 413 | A |
| 414 | A |
| 415 | A |
| 416 | A |
| 417 | A |
| 418 | A |
| 419 | A |
| 420 | A |
| 421 | A |
| 422 | A |
| 423 | A |
| 424 | A |
| 425 | A |
| 426 | A |
| 427 | A |
| 428 | A |
| 429 | A |
| 430 | A |
| 431 | A |
| 432 | A |
| 433 | A |
| 434 | A |
| 435 | A |
| 436 | A |
| 437 | A |
| 438 | A |
| 439 | A |
| 440 | A |
| 441 | A |
| 442 | A |
| 443 | A |
| 444 | A |
| 445 | A |
| 446 | A |
| 447 | A |
| 448 | A |
| 449 | A |
| 450 | A |
| 451 | A |
| 452 | A |
| 453 | A |
| 454 | A |
| 455 | A |
| 456 | A |
| 457 | A |
| 458 | A |
| 459 | A |
| 460 | A |
| 461 | A |
| 462 | A |
| 463 | A |
| 464 | A |
| 465 | A |
| 466 | A |
| 467 | A |
| 468 | A |
| 469 | A |
| 470 | A |
| 471 | A |
| 472 | A |
| 473 | A |
| 474 | A |
| 475 | A |
| 476 | A |
| 477 | A |
| 478 | A |
| 479 | A |
| 480 | A |
| 481 | A |
| 482 | A |
| 483 | A |
| 484 | A |
| 485 | A |
| 486 | A |
| 487 | A |
| 488 | A |
| 489 | A |
| 490 | A |
| 491 | A |
| 492 | A |
| 493 | A |
| 494 | A |
| 495 | A |
| 496 | A |
| 497 | B |
| 498 | A |
| 499 | A |
| 500 | A |
| 501 | A |
| 502 | A |
| 503 | B |
| 504 | A |
| 505 | A |
| 506 | A |
| 507 | A |
| 508 | A |
| 509 | A |
| 510 | A |
| 511 | A |
| 512 | A |
| 513 | A |
| 514 | C |
| 515 | A |
| 516 | A |
| 517 | A |
| 518 | A |
| 519 | A |
| 520 | A |
| 521 | A |
| 522 | A |
| 523 | A |
| 524 | A |
| 525 | A |
| 526 | A |
| 527 | A |
| 528 | A |
| 529 | A |
| 530 | A |
| 531 | A |
| 532 | A |
| 533 | A |
| 534 | A |
| 535 | A |
| 536 | A |
| 537 | A |
| 538 | A |
| 539 | A |
| 540 | A |
| 541 | A |
| 542 | A |
| 543 | A |
| 544 | A |
| 545 | A |
| 546 | A |
| 547 | A |
| 548 | B |
| 549 | B |
| 550 | A |
| 551 | A |
| 552 | A |
| 553 | A |
| 554 | B |
| 555 | B |
| 556 | A |
| 557 | A |
| 558 | B |
| 559 | B |
| 560 | A |
| 561 | A |
| 562 | A |
| 563 | A |
| 564 | A |
| 565 | A |
| 566 | A |
| 567 | A |
| 568 | A |
| 569 | A |
| 570 | A |
| 571 | A |
| 572 | A |
| 573 | A |
| 574 | A |
| 575 | A |
| 576 | A |
| 577 | A |
| 578 | A |
| 579 | A |
| 580 | A |
| 581 | A |
| 582 | A |
| 583 | A |
| 584 | A |
| 585 | A |
| 586 | A |
| 587 | A |
| 588 | B |
| 589 | B |
| 590 | C |
| 591 | A |
| 592 | A |
| 593 | A |
| 594 | A |
| 595 | A |
| 596 | A |
| 597 | A |
| 598 | A |
| 599 | A |
| 600 | A |
| 601 | A |
| 602 | A |
| 603 | A |
| 604 | A |
| 605 | A |
| 606 | A |
| 607 | A |
| 608 | A |
| 609 | A |
| 610 | A |
| 611 | A |
| 612 | B |
| 613 | A |
| 614 | A |
| 615 | B |
| 616 | A |
| 617 | A |
| 618 | A |
| 619 | A |
| 620 | A |
| 621 | B |
| 622 | A |
| 623 | B |
| 624 | B |
| 625 | B |
| 626 | A |
| 627 | C |
| 628 | A |
| 629 | C |
| 630 | A |
| 631 | C |
| 632 | A |
| 633 | A |
| 634 | B |

A = (IC₅₀ <100 nM);
B = (100 nM < IC₅₀ < 500 nM);
C = (500 nM < IC₅₀ < 5 μM)

### Cellular Cap-Dependent Translation Inhibition Assay

The effect of compounds on cellular cap-dependent translation was assessed using a stably integrated Flp-In™-293 (ThermoFisher Scientific, R75007) reporter cell-based dual luciferase assay (DLA), wherein the cap-dependent translation of unstable Firefly luciferase (Fluc-PEST) and cap-independent, poliovirus IRES-mediated translation of Renilla luciferase (Rluc) are measured after 24 hours of compound treatment. The reporter plasmid was constructed utilizing pcDNA5/FRT (Invitrogen, V601020). Test compounds were prepared using 11-point, 4-fold serial dilutions in DMSO and 100 nL of diluted compounds were transferred to 384-well assay-ready plates. Reporter cells were seeded to assay-ready plates in DMEM medium supplemented with 10% FBS at 10,000 cells in a volume of 33.5 microliters per well. After 24 hours of compound treatment, Fluc and Rluc activities were assessed sequentially using the Dual-Glo® Luciferase assay system (Promega, E2920) according to the manufacturer's instructions. Luminescence was measured using a Clariostar Plus microplate reader (BMG Labtech) or equivalent. For each well, the ratio of Fluc luminescence to Rluc luminescence was calculated. After normalization to the average of DMSO control and maximum inhibition control wells, concentration-response data were plotted and standard 4-parameter curve fitting (PEI Signals) was utilized to determine IC₅₀ values. Results of the cellular cap-dependent translation dual luciferase inhibition assay (DLA) are summarized in Table E4.

TABLE E4

| Compound | DLA 205 |
|---|---|
| No. | IC₅₀ |
| 1 | A |
| 2 | C |
| 3 | A |
| 4 | A |
| 5 | C |
| 6 | B |
| 7 | B |
| 8 | A |
| 9 | A |
| 10 | B |
| 11 | A |
| 12 | A |
| 13 | A |
| 14 | B |
| 15 | C |
| 16 | A |
| 17 | B |
| 18 | A |
| 19 | A |
| 20 | A |
| 21 | C |
| 22 | B |
| 23 | C |
| 24 | A |
| 25 | B |
| 26 | A |
| 27 | C |
| 28 | A |
| 29 | A |
| 30 | B |
| 31 | A |
| 32 | A |
| 33 | A |
| 34 | C |
| 35 | C |
| 36 | C |
| 37 | C |
| 38 | C |
| 39 | A |
| 40 | A |
| 41 | A |
| 42 | A |
| 43 | A |
| 44 | A |
| 45 | A |
| 46 | A |
| 47 | C |
| 48 | A |
| 49 | B |
| 50 | A |
| 51 | A |
| 52 | A |
| 53 | A |
| 54 | B |
| 55 | A |
| 56 | B |
| 57 | A |
| 58 | A |
| 59 | A |
| 60 | A |
| 61 | A |
| 62 | A |
| 63 | A |
| 64 | A |
| 65 | A |
| 66 | C |
| 67 | A |
| 68 | A |
| 69 | A |
| 70 | A |
| 71 | C |
| 72 | C |
| 73 | C |
| 74 | C |
| 75 | C |
| 76 | A |
| 77 | A |
| 78 | B |
| 79 | A |
| 80 | A |
| 81 | A |
| 82 | B |
| 83 | B |
| 84 | C |
| 85 | B |
| 86 | A |
| 87 | A |
| 88 | C |
| 89 | A |
| 90 | A |
| 91 | A |
| 92 | C |
| 93 | A |
| 94 | A |
| 95 | C |
| 96 | A |
| 97 | A |
| 98 | A |
| 99 | B |
| 241 | A |
| 242 | A |
| 243 | B |
| 244 | C |
| 245 | A |
| 246 | C |
| 247 | C |
| 248 | A |
| 249 | A |
| 250 | A |
| 251 | A |
| 252 | B |
| 253 | C |
| 254 | A |
| 255 | A |
| 256 | A |
| 257 | A |
| 259 | A |
| 260 | A |
| 261 | A |
| 262 | A |
| 263 | A |
| 264 | A |
| 265 | B |
| 266 | C |
| 267 | A |
| 268 | C |
| 269 | C |
| 270 | A |
| 271 | A |
| 272 | C |
| 273 | C |
| 274 | B |
| 275 | A |
| 276 | A |
| 277 | C |
| 278 | A |
| 279 | A |
| 280 | B |
| 281 | B |
| 282 | A |
| 283 | A |
| 284 | B |
| 285 | B |
| 286 | A |
| 287 | B |
| 288 | B |
| 289 | B |
| 290 | A |
| 291 | A |
| 292 | B |
| 293 | A |
| 294 | A |
| 295 | A |
| 296 | C |
| 297 | C |
| 298 | A |
| 299 | B |
| 300 | A |
| 301 | A |
| 302 | A |
| 303 | A |
| 304 | A |
| 305 | A |
| 306 | C |
| 307 | B |
| 308 | B |
| 309 | B |
| 310 | A |
| 311 | A |
| 312 | A |
| 313 | A |
| 314 | B |
| 315 | A |
| 316 | B |
| 317 | A |
| 318 | B |
| 319 | B |
| 320 | B |
| 321 | A |
| 322 | A |
| 323 | A |
| 324 | A |
| 325 | A |
| 326 | A |
| 327 | A |
| 328 | A |
| 329 | A |
| 330 | A |
| 331 | B |
| 332 | C |
| 333 | A |
| 334 | A |
| 335 | A |
| 336 | A |
| 337 | A |
| 338 | A |
| 339 | C |
| 340 | C |
| 341 | C |
| 342 | A |
| 343 | A |
| 344 | A |
| 345 | A |
| 346 | A |
| 347 | A |
| 348 | A |
| 349 | A |
| 350 | C |
| 351 | A |
| 352 | A |
| 353 | C |
| 354 | A |
| 355 | A |
| 356 | A |
| 357 | A |
| 358 | B |
| 359 | A |

A = IC₅₀ <1 μM;
B = 1 μM < IC₅₀ < 5 μM;
C = >5 μM

### Tumor Cell Viability Inhibition Assay

The effect of compound treatment on COLO 205 tumor cell viability was assessed using cellular ATP quantification methods (Promega CellTiter-Glo® 2.0, G9241), as follows. Test compounds were prepared using 11-point, 4-fold serial dilutions in DMSO and 100 nL of diluted compounds were transferred to 384-well assay-ready plates. COLO 205 (ATCC, CCL-222) colorectal tumor cells were seeded to assay-ready plates in RPMI-1640 medium supplemented with 10% FBS at 1500 cells in a volume of 33.5 microliters per well. After 72 hours of compound treatment, cellular ATP was assessed by CellTiter-Glo® 2.0 Cell Viability Assay method, according to the manufacturer's instructions. Luminescence was measured using a Clariostar Plus microplate reader (BMG Labtech) or equivalent. After normalization to the average of DMSO control and maximum inhibition control wells, concentration-response data were plotted and standard 4-parameter curve fitting (PEI Signals) was utilized to determine IC₅₀

TABLE E5

| Compound | COLO 205 |
|---|---|
| No. | IC₅₀ |
| 1 | B |
| 2 | A |
| 3 | A |
| 4 | B |
| 5 | B |
| 6 | B |
| 7 | B |
| 8 | B |
| 9 | A |
| 10 | B |
| 11 | A |
| 12 | A |
| 13 | A |
| 14 | B |
| 15 | C |
| 16 | A |
| 17 | B |
| 18 | A |
| 19 | A |
| 20 | A |
| 21 | C |
| 22 | C |
| 23 | C |
| 24 | B |
| 25 | C |
| 26 | A |
| 27 | C |
| 28 | A |
| 29 | A |
| 30 | C |
| 31 | A |
| 32 | A |
| 33 | A |
| 34 | C |
| 35 | C |
| 36 | C |
| 37 | B |
| 38 | C |
| 39 | A |
| 40 | A |
| 41 | A |
| 42 | A |
| 43 | A |
| 44 | A |
| 45 | A |
| 46 | A |
| 47 | C |
| 48 | A |
| 49 | B |
| 50 | A |
| 51 | A |
| 52 | A |
| 53 | A |
| 54 | B |
| 55 | A |
| 56 | B |
| 57 | A |
| 58 | A |
| 59 | A |
| 60 | A |
| 61 | A |
| 62 | A |
| 63 | A |
| 64 | A |
| 65 | A |
| 66 | C |
| 67 | A |
| 68 | A |
| 69 | A |
| 70 | A |
| 71 | C |
| 72 | C |
| 73 | C |
| 74 | C |
| 75 | B |
| 76 | A |
| 77 | B |
| 78 | C |
| 79 | A |
| 80 | A |
| 81 | A |
| 82 | C |
| 83 | B |
| 84 | C |
| 85 | A |
| 86 | A |
| 87 | A |
| 88 | C |
| 89 | A |
| 90 | A |
| 91 | A |
| 92 | C |
| 93 | A |
| 94 | A |
| 95 | C |
| 96 | A |
| 97 | A |
| 98 | A |
| 99 | B |
| 100 | A |
| 101 | A |
| 102 | B |
| 103 | B |
| 104 | A |
| 105 | A |
| 106 | A |
| 107 | A |
| 108 | A |
| 109 | A |
| 110 | B |
| 111 | A |
| 112 | B |
| 113 | A |
| 114 | A |
| 115 | B |
| 116 | A |
| 117 | A |
| 118 | C |
| 119 | B |
| 120 | A |
| 121 | A |
| 122 | A |
| 123 | C |
| 124 | A |
| 125 | A |
| 126 | A |
| 127 | A |
| 128 | A |
| 129 | A |
| 130 | A |
| 131 | A |
| 132 | A |
| 133 | B |
| 134 | A |
| 135 | A |
| 136 | A |
| 137 | A |
| 138 | B |
| 139 | A |
| 140 | A |
| 141 | A |
| 142 | B |
| 143 | A |
| 144 | B |
| 145 | B |
| 146 | A |
| 147 | A |
| 148 | A |
| 149 | A |
| 150 | B |
| 151 | A |
| 152 | A |
| 153 | B |
| 154 | A |
| 155 | C |
| 156 | A |
| 157 | B |
| 158 | C |
| 159 | C |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | B |
| 174 | A |
| 175 | A |
| 176 | A |
| 177 | A |
| 178 | A |
| 179 | A |
| 180 | A |
| 181 | A |
| 182 | A |
| 183 | C |
| 184 | C |
| 185 | A |
| 186 | C |
| 187 | A |
| 188 | C |
| 189 | A |
| 190 | C |
| 191 | A |
| 192 | A |
| 193 | C |
| 194 | A |
| 195 | C |
| 196 | A |
| 197 | A |
| 198 | A |
| 199 | A |
| 200 | A |
| 201 | A |
| 202 | C |
| 203 | A |
| 204 | A |
| 205 | A |
| 206 | C |
| 207 | C |
| 208 | C |
| 209 | C |
| 210 | C |
| 211 | C |
| 212 | B |
| 213 | A |
| 214 | C |
| 215 | C |
| 216 | A |
| 217 | A |
| 218 | B |
| 219 | C |
| 220 | A |
| 221 | A |
| 222 | A |
| 223 | A |
| 224 | C |
| 225 | C |
| 226 | C |
| 227 | C |
| 228 | C |
| 229 | C |
| 230 | B |
| 231 | B |
| 232 | A |
| 233 | C |
| 234 | C |
| 235 | C |
| 236 | C |
| 237 | C |
| 238 | C |
| 239 | A |
| 240 | A |
| 241 | A |
| 242 | A |
| 243 | B |
| 244 | C |
| 245 | B |
| 246 | C |
| 247 | C |
| 248 | A |
| 249 | B |
| 250 | A |
| 251 | A |
| 252 | B |
| 253 | C |
| 254 | A |
| 255 | A |
| 256 | A |
| 257 | B |
| 258 | A |
| 259 | A |
| 260 | A |
| 261 | A |
| 262 | A |
| 263 | A |
| 264 | A |
| 265 | C |
| 266 | C |
| 267 | B |
| 268 | C |
| 269 | C |
| 270 | B |
| 271 | A |
| 272 | C |
| 273 | C |
| 274 | B |
| 275 | A |
| 276 | A |
| 277 | C |
| 278 | A |
| 279 | A |
| 280 | B |
| 281 | B |
| 282 | A |
| 283 | A |
| 284 | B |
| 285 | B |
| 286 | A |
| 287 | B |
| 288 | B |
| 289 | B |
| 290 | A |
| 291 | A |
| 292 | B |
| 293 | A |
| 294 | A |
| 295 | A |
| 296 | C |
| 297 | C |
| 298 | A |
| 299 | B |
| 300 | A |
| 301 | B |
| 302 | A |
| 303 | A |
| 304 | B |
| 305 | A |
| 306 | B |
| 307 | B |
| 308 | B |
| 309 | B |
| 310 | A |
| 311 | A |
| 312 | A |
| 313 | A |
| 314 | B |
| 315 | A |
| 316 | B |
| 317 | A |
| 318 | B |
| 319 | B |
| 320 | B |
| 321 | A |
| 322 | A |
| 323 | A |
| 324 | A |
| 325 | A |
| 326 | A |
| 327 | A |
| 328 | A |
| 329 | A |
| 330 | A |
| 331 | B |
| 332 | B |
| 333 | A |
| 334 | A |
| 335 | A |
| 336 | A |
| 337 | A |
| 338 | A |
| 339 | B |
| 340 | C |
| 341 | C |
| 342 | A |
| 343 | A |
| 344 | A |
| 345 | B |
| 346 | A |
| 347 | A |
| 348 | A |
| 349 | A |
| 350 | C |
| 351 | A |
| 352 | A |
| 353 | C |
| 354 | A |
| 355 | B |
| 356 | A |
| 357 | A |
| 358 | B |
| 359 | A |
| 360 | A |
| 361 | A |
| 362 | A |
| 363 | A |
| 364 | A |
| 365 | A |
| 366 | B |
| 367 | A |
| 368 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | B |
| 384 | A |
| 385 | A |
| 386 | A |
| 387 | A |
| 388 | A |
| 389 | A |
| 390 | A |
| 391 | A |
| 392 | A |
| 393 | B |
| 394 | A |
| 395 | B |
| 396 | A |
| 397 | B |
| 398 | C |
| 399 | A |
| 400 | A |
| 401 | A |
| 402 | A |
| 403 | B |
| 404 | B |
| 405 | B |
| 406 | B |
| 407 | A |
| 408 | B |
| 409 | C |
| 410 | B |
| 411 | A |
| 412 | A |
| 413 | B |
| 414 | A |
| 415 | A |
| 416 | B |
| 417 | A |
| 418 | A |
| 419 | A |
| 420 | B |
| 421 | A |
| 422 | B |
| 423 | B |
| 424 | A |
| 425 | B |
| 426 | B |
| 427 | A |
| 428 | A |
| 429 | A |
| 430 | A |
| 431 | A |
| 432 | A |
| 433 | A |
| 434 | A |
| 435 | A |
| 436 | A |
| 437 | B |
| 438 | A |
| 439 | A |
| 440 | B |
| 441 | B |
| 442 | B |
| 443 | A |
| 444 | A |
| 445 | A |
| 446 | C |
| 447 | B |
| 448 | A |
| 449 | A |
| 450 | A |
| 451 | A |
| 452 | A |
| 453 | C |
| 454 | A |
| 455 | A |
| 456 | B |
| 457 | A |
| 458 | A |
| 459 | B |
| 460 | A |
| 461 | A |
| 462 | A |
| 463 | A |
| 464 | A |
| 465 | A |
| 466 | A |
| 467 | A |
| 468 | A |
| 469 | A |
| 470 | A |
| 471 | A |
| 472 | A |
| 473 | A |
| 474 | A |
| 475 | A |
| 476 | A |
| 477 | A |
| 478 | A |
| 479 | A |
| 480 | A |
| 481 | A |
| 482 | A |
| 483 | A |
| 484 | A |
| 485 | A |
| 486 | C |
| 487 | C |
| 488 | A |
| 489 | A |
| 490 | A |
| 491 | A |
| 492 | A |
| 493 | A |
| 494 | A |
| 495 | A |
| 496 | A |
| 497 | C |
| 498 | A |
| 499 | A |
| 500 | A |
| 501 | A |
| 502 | A |
| 503 | B |
| 504 | A |
| 505 | A |
| 506 | A |
| 507 | A |
| 508 | A |
| 509 | C |
| 510 | C |
| 511 | A |
| 512 | A |
| 513 | A |
| 514 | C |
| 515 | A |
| 516 | A |
| 517 | A |
| 518 | A |
| 519 | C |
| 520 | A |
| 521 | B |
| 522 | A |
| 523 | A |
| 524 | A |
| 525 | A |
| 526 | A |
| 527 | C |
| 528 | C |
| 529 | A |
| 530 | A |
| 531 | A |
| 532 | A |
| 533 | A |
| 534 | A |
| 535 | A |
| 536 | A |
| 537 | B |
| 538 | A |
| 539 | A |
| 540 | A |
| 541 | A |
| 542 | A |
| 543 | C |
| 544 | A |
| 545 | A |
| 546 | A |
| 547 | A |
| 548 | C |
| 549 | C |
| 550 | A |
| 551 | A |
| 552 | A |
| 553 | A |
| 554 | B |
| 555 | B |
| 556 | A |
| 557 | A |
| 558 | B |
| 559 | B |
| 560 | A |
| 561 | A |
| 562 | A |
| 563 | A |
| 564 | A |
| 565 | A |
| 566 | A |
| 567 | A |
| 568 | A |
| 569 | A |
| 570 | A |
| 571 | A |
| 572 | A |
| 573 | A |
| 574 | A |
| 575 | A |
| 576 | A |
| 577 | A |
| 578 | A |
| 579 | A |
| 580 | A |
| 581 | A |
| 582 | A |
| 583 | A |
| 584 | B |
| 585 | A |
| 586 | A |
| 587 | A |
| 588 | B |
| 589 | C |
| 590 | C |
| 591 | A |
| 592 | A |
| 593 | A |
| 594 | A |
| 595 | A |
| 596 | A |
| 597 | A |
| 598 | A |
| 599 | B |
| 600 | A |
| 601 | A |
| 602 | A |
| 603 | A |
| 604 | A |
| 605 | A |
| 606 | A |
| 607 | A |
| 608 | A |
| 609 | A |
| 610 | A |
| 611 | A |
| 612 | B |
| 613 | A |
| 614 | A |
| 615 | C |
| 616 | A |
| 617 | A |
| 618 | A |
| 619 | A |
| 620 | A |
| 621 | C |
| 622 | C |
| 623 | C |
| 624 | B |
| 625 | B |
| 626 | B |
| 627 | C |
| 628 | A |
| 629 | B |
| 630 | B |
| 631 | C |
| 632 | A |
| 633 | B |
| 634 | C |

A= IC₅₀ <5 μM;
B = 5 μM < IC₅₀ < 20 μM;
C = >20 μM

### Cyclin D1 Cellular Inhibition Assay

The effect of compound treatment on cyclin D1 protein levels in COLO 205 cells was assessed, as follows. Test compound plates were prepared with a Tecan D300e Digital Dispenser as 11-point concentration-response curves with a final DMSO concentration of 1%. COLO 205 (ATCC, CCL-222) colorectal tumor cells were seeded to assay-ready plates in RPMI-1640 medium supplemented with 10% FBS at 20,000 cells in a volume of 6 microliters per well. After 6 hours of compound treatment, cells were lysed and Cyclin D1 protein levels were assessed by an HTRF assay (PerkinElmer #64CYCD1ITPEG), according to the manufacturer's protocol. Time-resolved fluorescence was measured using a Clariostar Plus microplate reader (BMG Labtech) or equivalent. After normalization to the average of DMSO control and maximum inhibition control wells, concentration-response data were plotted and standard 4-parameter curve fitting (GraphPad Prism v9.4.1) was utilized to determine IC₅₀ values. The results of the are summarized in Table E6 below.

TABLE E6

| | COLO 205 |
|---|---|
| | 6 h Cyclin D1 |
| Compound | TR-FRET |
| No. | IC₅₀ |
| 1 | B |
| 3 | A |
| 11 | A |
| 13 | A |
| 14 | C |
| 18 | A |
| 31 | A |
| 42 | A |
| 46 | A |
| 51 | A |
| 70 | A |
| 138 | C |
| 139 | A |
| 140 | A |
| 141 | A |
| 142 | C |
| 143 | A |
| 144 | C |
| 145 | C |
| 146 | A |
| 147 | A |
| 148 | A |
| 149 | A |
| 150 | C |
| 151 | A |
| 152 | A |
| 153 | B |
| 154 | A |
| 156 | A |
| 157 | B |
| 158 | C |
| 159 | C |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | C |
| 174 | A |
| 175 | A |
| 176 | A |
| 177 | A |
| 178 | A |
| 179 | A |
| 180 | A |
| 181 | A |
| 182 | A |
| 183 | C |
| 185 | A |
| 186 | C |
| 187 | B |
| 188 | C |
| 189 | A |
| 190 | C |
| 191 | A |
| 192 | A |
| 193 | C |
| 194 | A |
| 195 | C |
| 196 | A |
| 197 | A |
| 198 | A |
| 199 | A |
| 200 | A |
| 201 | A |
| 202 | B |
| 203 | A |
| 204 | A |
| 205 | A |
| 206 | C |
| 207 | C |
| 208 | C |
| 209 | C |
| 210 | C |
| 212 | B |
| 213 | A |
| 214 | C |
| 215 | C |
| 216 | A |
| 217 | A |
| 218 | A |
| 219 | C |
| 220 | A |
| 221 | A |
| 222 | A |
| 223 | A |
| 224 | C |
| 227 | C |
| 229 | C |
| 230 | B |
| 231 | B |
| 232 | A |
| 235 | C |
| 236 | C |
| 239 | A |
| 240 | B |
| 241 | A |
| 242 | A |
| 243 | B |
| 244 | C |
| 245 | B |
| 257 | B |
| 258 | A |
| 259 | A |
| 260 | A |
| 261 | B |
| 263 | A |
| 326 | A |
| 328 | A |

A = IC₅₀ <5 μM,
B = 5 μM < IC₅₀ < 20 μM, and
C = IC₅₀ >20 μM

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A compound of Formula I″: 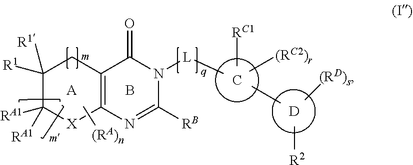
or a pharmaceutically acceptable salt, solvate, stereoisomer, or prodrug thereof,
wherein:
each -L- is independently —O—, —NR^{L}—, —CR^{L1}R^{L2}—, —CR^{L}═CR^{L2}—, or —C≡C—;
each R^{L} is independently hydrogen or optionally substituted C₁₋₆ alkyl;
each R^{L1} and each R^{L2} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted;
q is an integer selected from 1 to 5;
Ring C and Ring D are independently C₆₋₁₀ aryl or 5- to 10-membered heteroaryl;
R^{C1}, each R^{C2} and each R^{D} are independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
r and s are independently an integer selected from 0 to 6, as valency permits;
R² is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —C(═O)NR^{c}S(═O)₂R^{a}, —C(═O)NR^{c}R^{d}, —(CH₂)C(═O)OR^{b}, or —C(═O)OR^{b}, wherein the alkyl, alkenyl, alkynyl, alkoxy, alkylamino, carbocyclyl, heterocyclyl, aryl, or heteroaryl is optionally substituted,
R¹ is —NR^{1a}R^{1b} or —OR^{1c};
R^{1′} is hydrogen, deuterium, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
R¹ and R^{1′}, together with the carbon atom to which they are bonded, from C₃₋₁₂ carbocyclyl or 3- to 12-membered heterocyclyl, wherein the carbocyclyl or heterocyclyl is optionally substituted;
R^{1a} and R^{1b} are each independently hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted; or
R^{1a} and R^{1b}, together with the nitrogen atom to which they are bonded, form 3- to 12-membered heterocycle, wherein the heterocycle is optionally substituted with one or more R^{ab};
each R^{ab} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, 3- to 6-membered heterocyclyl, C₆₋₁₀ aryl, 5- to 10-membered heteroaryl, or —S(═O)R^{a}, wherein the alkyl, alkenyl, alkynyl, alkoxy, alkylamino, carbocyclyl, heterocyclyl, aryl, or heteroaryl is optionally substituted; or
two vicinal R^{ab}, together with the atoms to which they are bonded, form C₆ aryl or 5- to 6-membered heteroaryl, wherein the aryl or heteroaryl is optionally substituted;
R^{1c} is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted;
X is —O— or —C(R^{X})₂—;
each R^{X} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
two R^{X}, together with the carbon atom to which they are bonded, form an oxo; or
two R^{X}, together with the carbon atom to which they are bonded, form C₃₋₆ carbocyclyl, or 3- to 6-membered heterocyclyl, wherein the carbocyclyl or heterocyclyl is optionally substituted;
each R^{A1} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
R^{A1} and a vincinal R^{X}, together with the carbon atoms to which they are bonded, form C₃₋₄ carbocyclyl or 3- to 4-membered heterocyclyl, wherein the carbocyclyl or heterocyclyl is optionally substituted;
m and m′ are independently an integer selected from 0 to 2;
each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
n is an integer selected from 0 to 10, as valency permits;
R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
wherein:
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and each R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are bonded, form 3- to 12-membered heterocyclyl;
wherein each occurrence of R^{a}, R^{b}, R^{c}, and R^{d} is independently and optionally substituted.

2. The compound of claim 1, wherein Ring C is phenyl or pyridinyl.

3. The compound of claim 1, wherein the compound of Formula I″ is a compound of Formula I″-1-i, I″-1-ii, I″-1-iii, or I″-1-iv: 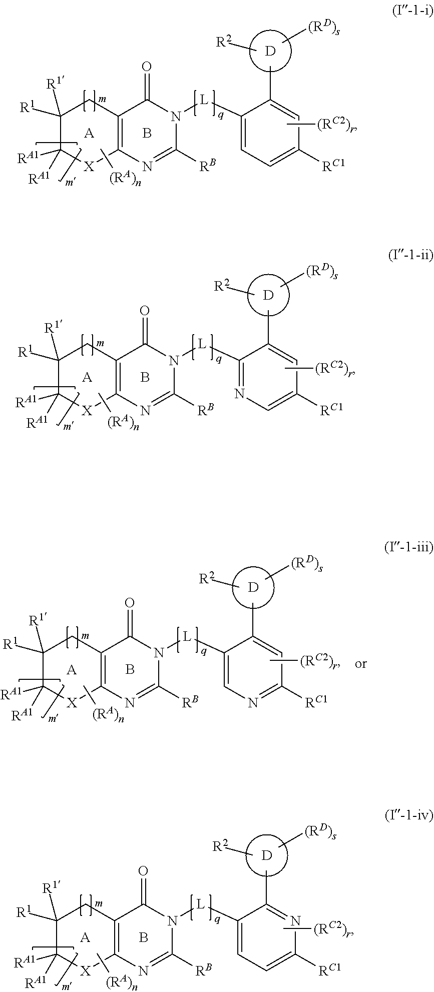
or a pharmaceutically acceptable salt, solvate, stereoisomer, or prodrug thereof.

4. The compound of claim 1, wherein:
(i) Ring D is phenyl, pyridinyl, pyrrolopyridazinyl, or thienopyridinyl; and/or
(ii) R² is hydrogen, —C(═O)NR^{c}S(═O)₂R^{a}, —C(═O)NR^{c}R^{d}, —(CH₂)C(═O)OR^{b}, or —C(═O)OR^{b}.

5. (canceled)

6. The compound of claim 4, wherein R² is —C(═O)NHS(═O)₂CH₃ or —COOH.

7. The compound of claim 1, wherein the compound of Formula I″ is a compound of Formula I″-1-i-1, I″-1-i-2, I″-1-i-3, I″-1-iii-1, I″-1-iii-2, or I″-1-iii-3: 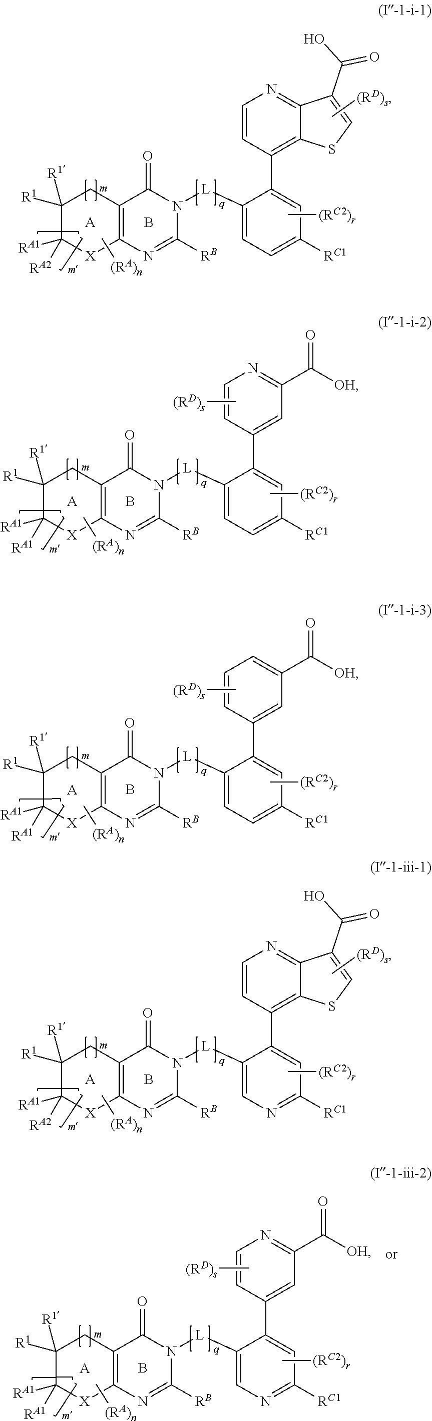 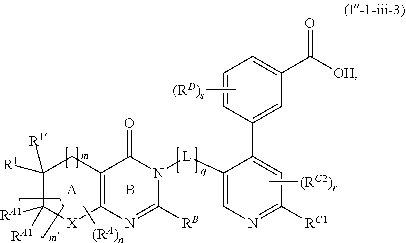
or a pharmaceutically acceptable salt, solvate, stereoisomer, or prodrug thereof.

8. The compound of claim 1, wherein:
(i) R^{C1} is halogen or —OH;
(ii) r is 0 or 1;
(iii) at least one R^{D} is C₁₋₆ alkyl or each of R^{D} is C₁₋₆ alkyl; and/or
(ii) s is 0, 1, or 2.

9.-12. (canceled)

13. The compound of claim 1, wherein [L]_{q} is 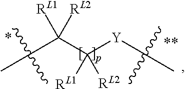
wherein:
* denotes attachment to Ring B, and ** denotes attachment to Ring C;
p is an integer selected from 0 to 3; and
Y is —O—, —NR^{L}—, —CR^{L1}R^{L2}—, or —C≡C—.

14. The compound of claim 13, wherein;
(i) each R^{L1} and each R^{L2} is hydrogen;
(ii) p is 0 or 1; and/or
(iii) Y is —O— or —C≡C—.

15.-16. (canceled)

17. The compound of claim 1, wherein:
(i) each of m and m′ is 1;
(ii) at least one R^{A} is C₁₋₆ alkyl;
(iii) n is 0, 1, or 2;
(iv) R^{B} is optionally substituted C₁₋₆ alkyl;
(v) each R^{A1} is hydrogen; and/or
(vi) X is —C(R^{X})₂—; and/or
(vii) R¹ is —NR^{1a}R^{1b}.

18.-21. (canceled)

22. The compound of claim 17, wherein;
(i) each R^{X} is independently hydrogen or C₁₋₆ alkyl;
(ii) two R^{X}, together with the carbon atom to which they are bonded, form C₃₋₄ carbocyclyl or 3- to 4-membered heterocyclyl; and/or
(iii) R^{X} and a vicinal R^{A1}, together with the carbon atoms to which they are bonded, form C₃₋₄ carbocyclyl or 3- to 4-membered heterocyclyl.

23.-26. (canceled)

27. The compound of claim 17, wherein:
(i) R^{1a} and R^{1b} are each independently hydrogen, —CN, C₁₋₆ alkyl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), or —(C₁₋₆ alkylene)-(3- to 12-membered heterocyclyl), wherein the alkyl, alkylene, carbocyclyl, heterocyclyl, aryl, or heteroaryl is optionally substituted;
(ii) at least one of R^{1a} and R^{1b} is not hydrogen; and/or
(iii) R^{1a} and R^{1b}, together with the nitrogen atom to which they are bonded, form 3- to 12-membered heterocycle, wherein the heterocycle is optionally substituted one or more R^{ab}.

28.-29. (canceled)

30. The compound of claim 27, wherein;
(i) each R^{ab} is independently oxo, halogen, —OH, C₁₋₆ alkyl, C₁₋₆ alkoxy, C₃₋₆ carbocyclyl, 3- to 6-membered heterocyclyl, C₆₋₁₀ aryl, 5- to 10-membered heteroaryl, or —S(═O)R^{a}, wherein the alkyl, alkenyl, alkynyl, alkoxy, alkylamino, carbocyclyl, heterocyclyl, aryl, or heteroaryl is optionally substituted; or
(ii) two vicinal R^{ab}, together with the atoms to which they are bonded, form C₆ aryl or 5- to 6-membered heteroaryl, wherein the aryl or heteroaryl is optionally substituted.

31. (canceled)

32. The compound of claim 1, wherein R¹ is —OR^{1c}.

33. The compound of claim 32, wherein R^{1c} is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted.

34. The compound of claim 1, wherein:
(i) R^{1′} is hydrogen, deuterium, or optionally substituted C₁₋₆ alkyl; or
(ii) R¹ and R^{1′}, together with the carbon atom to which they are bonded, from C₃₋₆ carbocyclyl or 3- to 6-membered heterocyclyl, wherein the carbocyclyl or heterocyclyl is optionally substituted.

35. (canceled)

36. A compound selected from Table 1, or a pharmaceutically acceptable salt, solvate, stereoisomer, or prodrug thereof.

37. A pharmaceutical composition comprising the compound of claim 1, and a pharmaceutically acceptable excipient.

38. A method of inhibiting a protein in a subject or biological sample comprising administering the compound of claim 1 to the subject or contacting the biological sample with the compound of claim 1.

39.-41. (canceled)

42. A method of treating or preventing a disease or disorder a subject in need thereof, comprising administering to the subject the compound of claim 1.

43.-45. (canceled)
